SEQUENCE LISTING (401 sequences)

SEQ ID NO: 1 - protein, 303 aa, Homo sapiens
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 2 - DNA, 910 bp, Homo sapiens
  source 1..910: mol_type = other DNA
atggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagt

SEQ ID NO: 3 - protein, 326 aa, Homo sapiens
MEKDGLCRADQQYECVAEIGEGAYGKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTA

SEQ ID NO: 4 - DNA, 11654 bp, Homo sapiens
  source 1..11654: mol_type = other DNA
actgcgtcccgcgccgctcgctcatccccgaggggcccctgcaacctctccgcgcgaagacggcttcagccctgcagggaaagaaaagtgcaatgattctggactgagacgcgcttgggcagaggctatgtaatcgtgtctgtgttgaggacttcgcttcgaggagggaagaggagggatcggctcgctcctccggcggcggcggcggcggcgactctgcaggcggagtttcgcggcggcggcaccagggttacgccagccccgcggggaggtctctccatccagcttctgcagcggcgaaagccccagcgcccgagcgcctgagccggcggggagcaagtaaagctagaccgatctccggggagccccggagtaggcgagcggcggccgccagctagttgagcgcaccccccgcccgccccagcggcgccgcggcgggcggcgtccaggcggcatggagaaggacggcctgtgccgcgctgaccagcagtacgaatgcgtggcggagatcggggagggcgcctatgggaaggtgttcaaggcccgcgacttgaagaacggaggccgtttcgtggcgttgaagcgcgtgcgggtgcagaccggcgaggagggcatgccgctctccaccatccgcgaggtggcggtgctgaggcacctggagaccttcgagcaccccaacgtggtcaggttgtttgatgtgtgcacagtgtcacgaacagacagagaaaccaaactaactttagtgtttgaacatgtcgatcaagacttgaccacttacttggataaagttccagagcctggagtgcccactgaaaccataaaggatatgatgtttcagcttctccgaggtctggactttcttcattcacaccgagtagtgcatcgcgatctaaaaccacagaacattctggtgaccagcagcggacaaataaaactcgctgacttcggccttgcccgcatctatagtttccagatggctctaacctcagtggtcgtcacgctgtggtacagagcacccgaagtcttgctccagtccagctacgccacccccgtggatctctggagtgttggctgcatatttgcagaaatgtttcgtagaaagcctctttttcgtggaagttcagatgttgatcaactaggaaaaatcttggacgtgattggactcccaggagaagaagactggcctagagatgttgcccttcccaggcaggcttttcattcaaaatctgcccaaccaattgagaagtttgtaacagatatcgatgaactaggcaaagacctacttctgaagtgtttgacatttaacccagccaaaagaatatctgcctacagtgccctgtctcacccatacttccaggacctggaaaggtgcaaagaaaacctggattcccacctgccgcccagccagaacacctcggagctgaatacagcctgaggcctcagcagccgccttaagctgatcctgcggagaacacccttggtggcttatgggtccccctcagcaagccctacagagctgtggaggattgctatctggaggccttccagctgctgtcttctggacaggctctgcttctccaaggaaaccgcctagtttactgttttgaaatcaatgcaagagtgattgcagctttatgttcatttgtttgtttgtttgtctgtttgtttcaagaacctggaaaaattccagaagaagagaagctgctgaccaattgtgctgccatttgatttttctaaccttgaatgctgccagtgtggagtgggtaatccaggcacagctgagttatgatgtaatctctctgcagctgccgggcctgatttggtacttttgagtgtgtgtgtgcatgtgtgtgtgtgtgtgtgtgtgtgtgtgtgtgtatgtgagagattctgtgatcttttaaagtgttactttttgtaaacgacaagaataattcaattttaaagactcaaggtggtcagtaaataacaggcatttgttcactgaaggtgattcaccaaaatagtcttctcaaattagaaagttaaccccatgtcctcagcatttcttttctggccaaaagcagtaaatttgctagcagtaaaagatgaagttttatacacacagcaaaaaggagaaaaaattctagtatattttaagagatgtgcatgcattctatttagtcttcagaatgctgaatttacttgttgtaagtctattttaaccttctgtatgacatcatgctttatcatttcttttggaaaatagcctgtaagctttttattacttgctataggtttagggagtgtacctcagatagattttaaaaaaaagaatagaaagcctttatttcctggtttgaaattcctttcttccctttttttgttgttgttattgttgtttgttgttgttattttgtttttgtttttaggaatttgtcagaaactctttcctgttttggtttggagagtagttctctctaactagagacaggagtggccttgaaattttcctcatctattacactgtactttctgccacacactgccttgttggcaaagtatccatcttgtctatctcccggcacttctgaaatatattgctaccattgtataactaataacagattgcttaagctgttcccatgcaccacctgtttgcttgctttcaatgaacctttcataaattcgcagtctcagcttatggtttatggcctcgattctgcaaacctaacagggtcacatatgttctctaatgcagtccttctacctggtgtttacttttgttacctaaataatgagtaggatcttgttttgttttatcaccagcacacagattgctataaactgttactttgtgaattacatttttatagaagatattttcagtgtctttacctgagggtatgtctttagctatgttttagggccatacatttactctatcaaatgatcttttctccatcccccaggctgtgcttatttctagtgccttgtgctcactcctgctctctacagagccagcctggcctgggcattgtaaacagcttttcctttttctcttactgttttctctacagtcctttatatttcataccatctctgccttataagtggtttagtgctcagttggctctagtaaccagaggacacagaaagtatcttttggaaagtttagccacctgtgctttctgactcagagtgcatgcaacagttagatcatgcaacagttagattatgtttagggttaggattttcaaagaatggaggttgctgcactcagaaaataattcagatcatgtttatgcattattaagttgtactgaattctttgcagcttaatgtgatatatgactatcttgaacaagagaaaaaactaggagatgtttctcctgaagagcttttggggttgggaactattcttttttaattgctgtactacttaacattgttctaattcagtagcttgaggaacaggaacattgttttctagagcaagataataaaggagatgggccatacaaatgttttctactttcgttgtgacaacattgattaggtgttgtcagtactataaatgcttgagatataatgaatccacagcattcaaggtcaggtctactcaaagtctcacatggaaaagtgagttctgcctttcctttgatcgagggtcaaaatacaaagacatttttgctagggcctacaaattgaatttaaaaactcactgcactgattcatctgagctttttggttagtattcatggctagagtgaacatagctttagtttttgctgttgtaaaagtgttttcataagttcactcaagaaaaatgcagctgttctgaactggaatttttcagcattctttagaattttaaatgagtagagagctcaacttttattcctagcatctgcttttgactcatttctaggcagtgcttatgaagaaaaattaaagcacaaacattctggcattcaatcgttggcagattatcttctgatgacacagaatgaaagggcatctcagcctctctgaactttgtaaaaatctgtccccagttcttccatcggtgtagttgttgcatttgagtgaatactctcttgatttatgtattttatgtccagattcgccatttctgaaatccagatccaacacaagcagtcttgccgttagggcattttgaagcagatagtagagtaagaacttagtgactacagcttattcttctgtaacatatggtttcaaacatctttgccaaaagctaagcagtggtgaactgaaaagggcatattgccccaaggttacactgaagcagctcatagcaagttaaaatattgtgacagatttgaaatcatgtttgaatttcatagtaggaccagtacaagaatgtccctgctagtttctgtttgatgtttggttctggcggctcaggcattttgggaactgttgcacagggtggagtcaaaacaacctacatataaaaagagaaaaagagaaacttgtccatttagctttcataagaaatcccatggcaaagggtaataaaaaggacctaatcttaaaaatacaatttctaagcacttgtaagaacccagtgggttggagcctcccactttgtccctcctttgaagtggatgggaactcaaggtgcaaagaacctgttttggaagaaagcttggggccatttcagccccctgtattctcatgattttctctcaggaagcacacactgtgaatggcagacttttcatttagccccaggtgacttactaaaaatagttgaaaattattcacctaagaatagaatctcagcattgtgttaaataaaaatgaaagctttagaaggcatgagatgttcctatcttaaataaagcatgtttcttttctatagagaaatgtatagtttgactctccagaatgtactatccatcttgatgagaaaactcttaaatagtaccaaacattttgaactttaaattatgtatttaaagtgagtgtttaagaaactgtagctgcttcttttacaagtggtgcctattaaagtcagtaatggccattattgttccattgtggaaattaaattatgtaagcttcctaatatcataaacatattaaaattcttctaaaatattgcttttcttttaagtgacaatttgactattcttatgataagcacatgagagtgtcttacattttccaaaagcaggctttaattgcatagttgagtctaggaaaaaataatgttaaaagtgaatatgccaccataattacttaattatgttagtatagaaactacagaatatttaccctggaaagaaaatattggaatgttattataaactcttagatatttatataattcaaaagaatgcatgtttcacattgtgacagataaagatgtatgatttctaaggctttaaaaattattcataaaacagtgggcaatagataaaggaaattctggagaaaatgaaggtatttaaagggtagtttcaaagctatatatattttgaaggatatattctttatgaacaaatatattgtaaaaatttatactaaggtcatctggtaactgtgggattaatatggtcgaaaacaaatgttatggagaagctgtcccaagcaaactaaattacctgtacttttttcccatttcaagggaagaggcaaccacatgaagcaatacttcttacacatgcctaagaacgttcattgaaaaaataaatttttaaaaggcatgtgtttcctatgccaccaatacttttgaaaaattgtgaaccttacccaaaaccatttatcatgtccattaagtatatttgggtatataattaggaagatatttacatgttccatctccacagtggaaaaacttattgaggctaccaaagtgtgccaagaaatgtaagtccttagagtaattagaaatgctgttttcctcaaaagcatgagaaactagcattttcatttcttatttactccctttctatatcaatgcaattcacaacccaattttaatacatccctatatctcaagcatttctatcttgtactttttcagaaaataaaccaaaaataatcctttggtctctctatcttctgacctttgtaagcaacagaaatgtaaaaacagaaggggtccaatttttacacgtttttttctcaagtagcctttctggggatttttattttcttaatgaagtgccaatcagcttttcaaaatgttttctatttctcagcatttccaggaagtgataacgtttagctaaatgagtagaagtggacttccttcaacatattgttaccttgtctagccttaggaagaaaacaagagccacctgaaaataaatacaggctcttttcgagcatctgctgaaatactgttacagcaatttgaagttgatgtggtaggaaaggaaggtgacttttcttgcaaaagtctttctaaacattcacactgtcctaagagatgagctttcttgttttattccggtatattccacaaggtggcacttttagagaaaaacaaatctgatgaagactaaagaggtacttctaaaagagatttcattctaactttatttttctgcgcatatttaactctttcctagcacttgttttttgggatgattaatagtctctataatgttctgtaacttcaatattttacttgttacctaggttctgaacaattgtctgcaaataaattgttcttaaggatggataatacacccattttgatcatttaagtaaagaaagcctagtcattcattcagtcaagaaaaaatttttgaagtacccagttaccttacttttctagattaaaacaggcttagttactaaaaaggcagtcctcatctgtgaacaggatagtttcgttagaagtataaaactcctttagtggccccagttaaaacacacataccctctctgctgctttcaaattccctagcatggtggcctttcaacattgattaaattttaaaatcctaatttaaagatcaggtgagcaaaatgagtagcacatcagtaattcagtagacaaaacttttgtctgaaaaattgctgtattgaaacagagccctaaaataccaaaagaccaggtaattttaacatttgtggaatcacaaatgtaaattcataagaagctctaattaaaaaaaaaaagtctgaagtatatgagcataacaacttaggagtgtgtctacatacttaacttttgaagttttttggcaactttatatactttttttaaatttacaagtctacttaaagacttcttataccccaaatgattaagttaattttagaggtcacctttctcacagcagtgtcacttgaaatttagtagggaaggatattgcagtatttttcagtttccttagcacagcaccacagaaagcagcttattccttttgagtggcagacactcgacggtgcctgcccaactttcctcctgagtggcaagcagatgagtctcagtaattcatactgaaccaaaatgccacatacactaggggcagtcagaaactggctgagaaatcccccgcctcattcgcccctctgctcccaggaactagagtccagttaaagcccctatgcgaaaggccgaattccaccccagggtttgttataacagtggccagtctgaaccccatttgctcgtgctcaaaacttgattcccacttgaaagccttccgggcgcgctgcctcgttgccccgcccctttggcaggagagaggcagtgggcgaggccgggctggggccccgcctcccactcacctgccggtgcctgaaattatgtgcggccccgcgggctgctttccgaggtcagagtgccctgctgctgtctcagaggcatctgttctgcaaatcttaggaagaaaaatgtccctagtagcaaacgggtgtcttctgtgcataaataagtacaacacaattctccgaaagttcgggtaaaaagagatgcggtagcagctgccctgtgtgaagctgtctaccccgcatctctcaggcgctaagctcagtttttgtttttgtttttgtttttttaaagaaaagatgtataattgcaggaatttttttttatttttttattttccatcattctatatatgtgatggtgaaagatatgcctggaaaagttttgttttgaaaagtttattttctgcttcgtcttcagttggcaaaagctctcaattctttagcttccagtttcttttctctctttttctttgttaggtaattaaaggtatgtaaacaaattatctcatgtagcaggggattttcatgttgagaggaatcttccgtgtgagttgtttggtcacacaaataaccctttctcaattttaggagtttggattgtcaaatgtaggtttttctcaaagggggcatataactacatattgactgccaagaactatgactgtagcactaatcagcacacatagagccacacaattatttaatttctaactctctgtggtccctagaaaaattccgttgatgtgcttaggttaaagttctgaagatacccgttgtacccttacttgaaagtttctaatcttaagttttatgaaatgcaataatatgtatcagctagcaatatttctgtgatcaccaacaactctcagtttgatcttaaagtctgaataataaaacaaatcccagcagtaatacatttcttaaacctcacagtgcatgatatatcttttcattctgatcctgtgtttgcaaaaatatacacatgtatatcatagttcctcactttttattcatttgttttcctattacctgtagtaaatatattagttagtacatggaatttatagcatcagctacccccaggaacagcacctgacaggcgggggattttttttcaagttgttctacatttgcataaattatttctattattattcatgtatgttatttatttctgaatcacactagtcctgtgaaagtacaactgaaggcagaaagtgttaggattttgcatctaatgttcattatcatggtattgatggacctaagaaaataaaaattagactaagcccccaaataagctgcatgcatttgtaacatgattagtagatttgaatatatagatgtagtattttgggtatctaggtgttttatcattatgtaaaggaattaaagtaaaggactttgtagttgtttttattaaatatgcatatagtagagtgcaaaaatatagcaaaaataaaaactaaaggtagaaaagcattttagatatgccttaatttagaaactgtgccaggtggccctcggaatagatgccaggcagagaccagtgcctgggtggtgcctcctcttgtctgccctcatgaagaagcttccctcacgtgatgtagtgccctcgtaggtgtcatgtggagtagtgggaacaggcagtactgttgagaggagagcagtgtgagagtttttctgtagaagcagaactgtcagcttgtgccttgaggcttccagaacgtgtcagatggagaagtccaagtttccatgcttcaggcaacttagctgtgtacagaagcaatccagtgtggtaataaaaagcaaggattgcctgtataatttattataaaataaaagggattttaacaaccaacaattcccaacacctcaaaagcttgttgcattttttggtatttgaggtttttatctgaaggttaaagggcaagtgtttggtatagaagagcagtatgtgttaagaaaagaaaaatattggttcacgtagagtgcaaattagaactagaaagttttatacgattatcattttgagatgtgttaaagtaggttttcactgtaaaatgtattagtgtttctgcattgccatagggcctggttaaaactttctcttaggtttcaggaagactgtcacatacagtaagcttttttccttctgacttataatagaaaatgttttgaaagtaaaaaaaaaaaatctaatttggaaatttgacttgttagtttctgtgtttgaaatcatggttctagaaatgtagaaattgtgtatatcagatactcatctaggctgtgtgaaccagcccaagatgaccaacatccccacacctctacatctctgtcccctgtatctcttcctttctaccactaaagtgttccctgctaccatcctggcttgtccacatggtgctctccatcttcctccacatcatggaccacaggtgtgcctgtctaggcctggccaccactcccaacttgacctagccacattcatctagagatggttcctgatgctgggcacagactgtgctcatggcacccattagaaatgcctctagcatctttgtatgcatcttgatttttaaaccaagtcattgtacagagcattcagttttggctgtggtaccaagagaaaaactaatcaagaatataaaccacattccaggctgctgttttctctccatctacaggccacacttttactgtatttcttcatacttgaaattcattctgctattttcatatcagggtacagacttataagggtgcatgttccttaaaggtgcataattattcttattccgtttgcttatattgctacagaatgctctgttttggtgctttgagttctgcagacccaagaagcagtgtggaaattcactgcctgggacacagtcttataagaatgttggcaggtgactttgtatcagatgttgcttctcttttctctgtacacagattgagagttaccacagtggcctgtcgggtccaccctgtgggtgcagcacagctctctgaaagcaagaaccttcctacctattctaacgtttttgccctctaagaaaaatggcctcaggtatggtatagacatagcaagaggggaagggctgtctcactctagcaaccatccctccattacacacagaaagccctcttgaagcaaaagaagaagaaagaaagaaagcttatctctaaggctactgtcttcagaatgctctgagctgaatgctcttgctcctttcccaagaggcagatgaaaatatagccagtttatctatacccttcctatctgaggaggagaatagaaaagtagggtaaatatgtaacgtaaaatatgtcattcaaggaccaccaaaactttaagtaccctatcattaaaaatctggttttaaaagtagctcaagtaagggatgctttgtgacccagggtttctgaagtcagatagccattcttacctgccccttactctgacttattgggaaagggagaactgcagtggtgtttctgttgcagtggcaaaggtaacatgtcagaaaattcagagggttgcataccaataatcctttggaaactggatgtcttactgggtgctagaatgaaaatgtaggtatttattgtcagatgatgaagttcattgtttttttcaaaattggtgttgaaatatcactgtccaatgtgttcacttatgtgaaagctaaattgaatgaggcaaaaagagcaaatagtttgtatatttgtaataccttttgtatttcttacaataaaaatattggtagcaaataaaaataataaaaacaataactttaaactgctttctggagatgaattactctcctggctattttcttttttactttaatgtaaaatgagtataactgtagtgagtaaaattcattaaattccaagttttagcagaa

SEQ ID NO: 5 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 6 - DNA, 6822 bp, Homo sapiens
  source 1..6822: mol_type = other DNA
agacacgtgctggggcgggcaggcgagcgcctcagtctggtcgcctggcggtgcctccggccccaacgcgcccgggccgccgcgggccgcgcgcgccgatgcccggctgagtcactggcagggcagcgcgcgtgtgggaaggggcggagggagtgcggccggcgggcgggcggggcgctgggctcagcccggccgcaggtgacccggaggccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaa

SEQ ID NO: 7 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 8 - DNA, 3633 bp, Homo sapiens
  source 1..3633: mol_type = other DNA
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaagcttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 9 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 10 - protein, 2097 aa, Homo sapiens
GCTCACCTCCGCCTGAGCAGTGGAGAAGGCGGCACTCTGGTGGGGCTGCTCCAGGCATGCAGATCCCACAGGCGCCCTGGCCAGTCGTCTGGGCGGTGCTACAACTGGGCTGGCGGCCAGGATGGTTCTTAGACTCCCCAGACAGGCCCTGGAACCCCCCCACCTTCTCCCCAGCCCTGCTCGTGGTGACCGAAGGGGACAACGCCACCTTCACCTGCAGCTTCTCCAACACATCGGAGAGCTTCGTGCTAAACTGGTACCGCATGAGCCCCAGCAACCAGACGGACAAGCTGGCCGCCTTCCCCGAGGACCGCAGCCAGCCCGGCCAGGACTGCCGCTTCCGTGTCACACAACTGCCCAACGGGCGTGACTTCCACATGAGCGTGGTCAGGGCCCGGCGCAATGACAGCGGCACCTACCTCTGTGGGGCCATCTCCCTGGCCCCCAAGGCGCAGATCAAAGAGAGCCTGCGGGCAGAGCTCAGGGTGACAGAGAGAAGGGCAGAAGTGCCCACAGCCCACCCCAGCCCCTCACCCAGGCCAGCCGGCCAGTTCCAAACCCTGGTGGTTGGTGTCGTGGGCGGCCTGCTGGGCAGCCTGGTGCTGCTAGTCTGGGTCCTGGCCGTCATCTGCTCCCGGGCCGCACGAGGGACAATAGGAGCCAGGCGCACCGGCCAGCCCCTGAAGGAGGACCCCTCAGCCGTGCCTGTGTTCTCTGTGGACTATGGGGAGCTGGATTTCCAGTGGCGAGAGAAGACCCCGGAGCCCCCCGTGCCCTGTGTCCCTGAGCAGACGGAGTATGCCACCATTGTCTTTCCTAGCGGAATGGGCACCTCATCCCCCGCCCGCAGGGGCTCAGCTGACGGCCCTCGGAGTGCCCAGCCACTGAGGCCTGAGGATGGACACTGCTCTTGGCCCCTCTGACCGGCTTCCTTGGCCACCAGTGTTCTGCAGACCCTCCACCATGAGCCCGGGTCAGCGCATTTCCTCAGGAGAAGCAGGCAGGGTGCAGGCCATTGCAGGCCGTCCAGGGGCTGAGCTGCCTGGGGGCGACCGGGGCTCCAGCCTGCACCTGCACCAGGCACAGCCCCACCACAGGACTCATGTCTCAATGCCCACAGTGAGCCCAGGCAGCAGGTGTCACCGTCCCCTACAGGGAGGGCCAGATGCAGTCACTGCTTCAGGTCCTGCCAGCACAGAGCTGCCTGCGTCCAGCTCCCTGAATCTCTGCTGCTGCTGCTGCTGCTGCTGCTGCTGCCTGCGGCCCGGGGCTGAAGGCGCCGTGGCCCTGCCTGACGCCCCGGAGCCTCCTGCCTGAACTTGGGGGCTGGTTGGAGATGGCCTTGGAGCAGCCAAGGTGCCCCTGGCAGTGGCATCCCGAAACGCCCTGGACGCAGGGCCCAAGACTGGGCACAGGAGTGGGAGGTACATGGGGCTGGGGACTCCCCAGGAGTTATCTGCTCCCTGCAGGCCTAGAGAAGTTTCAGGGAAGGTCAGAAGAGCTCCTGGCTGTGGTGGGCAGGGCAGGAAACCCCTCCACCTTTACACATGCCCAGGCAGCACCTCAGGCCCTTTGTGGGGCAGGGAAGCTGAGGCAGTAAGCGGGCAGGCAGAGCTGGAGGCCTTTCAGGCCCAGCCAGCACTCTGGCCTCCTGCCGCCGCATTCCACCCCAGCCCCTCACACCACTCGGGAGAGGGACATCCTACGGTCCCAAGGTCAGGAGGGCAGGGCTGGGGTTGACTCAGGCCCCTCCCAGCTGTGGCCACCTGGGTGTTGGGAGGGCAGAAGTGCAGGCACCTAGGGCCCCCCATGTGCCCACCCTGGGAGCTCTCCTTGGAACCCATTCCTGAAATTATTTAAAGGGGTTGGCCGGGCTCCCACCAGGGCCTGGGTGGGAAGGTACAGGCGTTCCCCCGGGGCCTAGTACCCCCGCCGTGGCCTATCCACTCCTCACATCCACACACTGCACCCCCACTCCTGGGGCAGGGCCACCAGCATCCAGGCGGCCAGCAGGCACCTGAGTGGCTGGGACAAGGGATCCCCCTTCCCTGTGGTTCTATTATATTATAATTATAATTAAATATGAGAGCATGCTAA

SEQ ID NO: 11 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 12 - DNA, 873 bp, Homo sapiens
  source 1..873: mol_type = other DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 13 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 14 - DNA, 1173 bp, Homo sapiens
  source 1..1173: mol_type = other DNA
atgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 15 - protein, 451 aa, Homo sapiens
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 16 - protein, 818 aa, Homo sapiens
MERYKALEQLLTELDDFLKILDQENLSSTALVKKSCLAELLRLYTKSSSSDEEYIYMNKVTINKQQNAESQGKAPEEQGLLPNGEPSQHSSAPQKSLPDLPPPKMIPERKQLAIPKTESPEGYYEEAEPYDTSLNEDGEAVSSSYESYDEEDGSKGKSAPYQWPSPEAGIELMRDARICAFLWRKKWLGQWAKQLCVIKDNRLLCYKSSKDHSPQLDVNLLGSSVIHKEKQVRKKEHKLKITPMNADVIVLGLQSKDQAEQWLRVIQEVSGLPSEGASEGNQYTPDAQRFNCQKPDIAEKYLSASEYGSSVDGHPEVPETKDVKKKCSAGLKLSNLMNLGRKKSTSLEPVERSLETSSYLNVLVNSQWKSRWCSVRDNHLHFYQDRNRSKVAQQPLSLVGCEVVPDPSPDHLYSFRILHKGEELAKLEAKSSEEMGHWLGLLLSESGSKTDPEEFTYDYVDADRVSCIVSAAKNSLLLMQRKFSEPNTYIDGLPSQDRQEELYDDVDLSELTAAVEPTEEATPVADDPNERESDRVYLDLTPVKSFLHGPSSAQAQASSPTLSCLDNATEALPADSGPGPTPDEPCIKCPENLGEQQLESLEPEDPSLRITTVKIQTEQQRISFPPSCPDAVVATPPGASPPVKDRLRVTSAEIKLGKNRTEAEVKRYTEEKERLEKKKEEIRGHLAQLRKEKRELKETLLKCTDKEVLASLEQKLKEIDEECRGEESRRVDLELSIMEVKDNLKKAEAGPVTLGTTVDTTHLENVSPRPKAVTPASAPDCTPVNSATTLKNRPLSVVVTGKGTVLQKAKEWEKKGAS

SEQ ID NO: 17 - protein, 343 aa, Homo sapiens
MESKYKEILLLTGLDNITDEELDRFKFFLSDEFNIATGKLHTANRIQVATLMIQNAGAVSAVMKTIRIFQKLNYMLLAKRLQEEKEKVDKQYKSVTKPKPLSQAEMSPAASAAIRNDVAKQRAAPKVSPHVKPEQKQMVAQQESIREGFQKRCLPVMVLKAKKPFTFETQEGKQEMFHATVATEKEFFFVKVFNTLLKDKFIPKRIIIIARYYRHSGFLEVNSASRVLDAESDQKVNVPLNIIRKAGETPKINTLQTQPLGTIVNGLFVVQKVTEKKKNILFDLSDNTGKMEVLGVRNEDTMKCKEGDKVRLTFFTLSKNGEKLQLTSGVHSTIKVIKAKKKT

SEQ ID NO: 18 - protein, 323 aa, Homo sapiens
MDSKYQCVKLNDGHFMPVLGFGTYAPAEVPKSKALEAVKLAIEAGFHHIDSAHVYNNEEQVGLAIRSKIADGSVKREDIFYTSKLWSNSHRPELVRPALERSLKNLQLDYVDLYLIHFPVSVKPGEEVIPKDENGKILFDTVDLCATWEAMEKCKDAGLAKSIGVSNFNHRLLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 19 - protein, 323 aa, Homo sapiens
MDPKYQRVELNDGHFMPVLGFGTYAPPEVPRNRAVEVTKLAIEAGFRHIDSAYLYNNEEQVGLAIRSKIADGSVKREDIFYTSKLWCTFFQPQMVQPALESSLKKLQLDYVDLYLLHFPMALKPGETPLPKDENGKVIFDTVDLSATWEVMEKCKDAGLAKSIGVSNFNCRQLEMILNKPGLKYKPVCNQVECHPYLNQSKLLDFCKSKDIVLVAHSALGTQRHKLWVDPNSPVLLEDPVLCALAKKHKQTPALIALRYQLQRGVVVLAKSYNEQRIRENIQVFEFQLTSEDMKVLDGLNRNYRYVVMDFLMDHPDYPFSDEY

SEQ ID NO: 20 - protein, 533 aa, Homo sapiens
MQVKNIRKLPCQIIQSVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRATIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 21 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 22 - protein, 466 aa, Homo sapiens
MSYPGYPPTGYPPFPGYPPAGQESSFPPSGQYPYPSGFPPMGGGAYPQVPSSGYPGAGGYPAPGGYPAPGGYPGAPQPGGAPSYPGVPPGQGFGVPPGGAGFSGYPQPPSQSYGGGPAQVPLPGGFPGGQMPSQYPGGQPTYPSQPATVTQVTQGTIRPAANFDAIRDAEILRKAMKGFGTDEQAIVDVVANRSNDQRQKIKAAFKTSYGKDLIKDLKSELSGNMEELILALFMPPTYYDAWSLRKAMQGAGTQERVLIEILCTRTNQEIREIVRCYQSEFGRDLEKDIRSDTSGHFERLLVSMCQGNRDENQSINHQMAQEDAQRLYQAGEGRLGTDESCFNMILATRSFPQLRATMEAYSRMANRDLLSSVSREFSGYVESGLKTILQCALNRPAFFAERLYYAMKGAGTDDSTLVRIVVTRSEIDLVQIKQMFAQMYQKTLGTMIAGDTSGDYRRLLLAIVGQ

SEQ ID NO: 23 - protein, 636 aa, Homo sapiens
MAAEEEAAAGGKVLREENQCIAPVVSSRVSPGTRPTAMGSFSSHMTEFPRKRKGSDSDPSQSGIMTEKVVEKLSQNPLTYLLSTRIEISASSGSRVEDGEHQVKMKAFREAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSMSNKELFPPSPSEMGELEATRQNQSTVAVHSHEPLLSDGAQLDFDALCDNDDTAMAAFMNYLEAEGGLGDPGDFSDIQWTL

SEQ ID NO: 24 - protein, 127 aa, Homo sapiens
MSQGLPAAGSVLQRSVAAPGNQPQPQPQQQSPEDDDRKVRRREKNRVAAQRSRKKQTQKADKLHEEYESLEQENTMLRREIGKLTEELKHLTEALKEHEKMCPLLLCPMNFVPVPPRPDPVAGCLPR

SEQ ID NO: 25 - protein, 108 aa, Homo sapiens
MAKQPSDVSSECDREGRQLQPAERPPQLRPGAPTSLQTEPQASMRQAEPADMRPEIWIAQELRRIGDEFNAYYARRVFLNNYQAAEDHPRMVILRLLRYIVRLVWRMH

SEQ ID NO: 26 - protein, 1362 aa, Homo sapiens
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 27 - protein, 2642 aa, Homo sapiens
AAATGGTGCCAGACGTGGTGGTATGAGCCTGTAATTCCAGCCACTCAGGAGGCTGAGGTGGGAGGATCACTCTGGCCTAGGAGTTTGAGATCAGCCTGGGTGACAGAGCAAGACCCTGTCTCTAGAAAAGAGAAAGAATAAATGGCTGATGGAGATGACTCTACTGCAAAAATGACAACCAGCAAGAAAGCGGGTGGTGCCCGTTTCCTCCCTGGGGACCCTCAGAGGGCCAGGCCATTCTCTTCCTTGGTGTGACTGCGGATCCAGGGATCTCGTGCCCCCAGTCTGAAGTGACTAGCGCAGAGCTGGGATTTTTTTGGCCCAGCTGAAGAGCATCAGATCTGTGGGCATGGTACGTTTGCTGAAGGACAGTGGCAGAGTTCCAGGGCAGGCAGGGCCGGCCCATGGTGTCAGTGCTGGCCTTTCCTCGTTGCACCAGTGAATGTTGGAGAAGTACTTGTTGGACACACATGTTCCTCCATTTAGACAGTCACAGTTCGCTGGAGAGAACAAAGCCAACTGTTGTAGGGGTGGATACTCACATGGAACTTGATGAAGTTCATTGCTGCCCTGGAAGAGATTCGGGAGGAGGCTTCATCAAAGGTCCCATGCTCCAGGGACTCCAGGGTGAGGGTAAACTGGCCCCAATCCCAAAACCCACTCTTCCCTCTCCCTCTCGCCTCACCCTGTTCGTATCTAGTTCTCAAATGGAAGACCATGGGTTTCCAGCCAGGAGAAATGGATTGACCCAAGCAAGCTTCATCTACCAGATGCCCGCAGGCTGGGGCAGTCCCGGCGGCCTCTTCCTCCCTTGCCAGCCAGTGCCAACCCCCGTGGTTCTCAAGCCTCCGCTGCCACCCTGTCCCATTTCCTGGGGAGAGTCTGGCCCTGCTGTGGATGGAATCCGGAGGACCCCAGCTCCCTGAGCAGCCCCTCCTCTCCCCTGGTGCTGATCAGAGGTCCTTGGGCAGCATCAGTCAAAGCAAGAGCGCACTCACTTTGGAGTCGCTCACGACCAGGACGCAGAGAAGCAGGCGCGCCAGCAGGGCTCTCATGGTGGCGAGGTCGGGGCGCTAGACGGCGGCTCTGCAAAGGAAGGAGAAGTCAGGGCAAGAGGCGGAGGAACGGGAAGGCAGGCCAGGCGGGCGACTGCAGCGCAGGGGAGATGCCCGCGGTGACCAGGCTCCCCAGCTGTCTCTCTCCTCTCTGGGCTCCGGACTCCGGGCAGCCTGGATCGGCACCCGCGGGGGACGCCCGGGACGGGGCGCCTTGACTCCGTGCAGCCGCCGGGGAGCCCAGGGAGCCCGGGCAGCCCAGGGCGGGGGAGGCAGACGCTCGGGAGCTGGGGCCGCCGCGCATCCGGCCCGGGGATCTCAGGACCGCGGCACTCACCGGTGGCTGCGGCAGGAGGGCGCGAGCCGGCGCTGCGGGGACAGGTGGACCCTGGCCCGGGCTCCGGGGCTGCGGTCTCCGCACTGTGCTGCGACCCGCGGCGCCTGCTCTATATCAGGGCCCGCCCCGGCGCCGCCCCTCCCTCTCCCGCCCGGCTCCCTCCCCTGTCTTGCAGCGCTCAGCGACCCGGACCCCGCGTGCTTCCGCAACGCTCACAAAGATTTGGGGGAAGCGCGATCTCCAGCGGAGGGGACCCAACAGCGTCTGGACTGAGGAATCGAGAGGCTTGTAAATTCTCCGTGCTTCCTCCCATGCACCTGGCCGGGGGCCTGCCCCAGTGCAAGGAGTCCCCGAATTGCAGAGAGGAGAGAAGGCGCACAGGAGACTCTCTACCTCGCCCAGCTCTGAAGCCTCCTGGGGTCCTCTAATCAGTTCTTCTGCAACTTCTCCCCGCTGGGCCCCAACTTGCCTAAGACTGCCTCAGACCCCCTTGCCCGCAGCTGATGGAGCTGTGAAGTCTTCATCAACGCGACAAATGTACGAGACATACTCTCCCAGAAGCACAGACAGAAAAACCCCTGCCTGTAGGGGCTCCCTCTGTGCGTCTGTTCAGTGGCAGTCCCCAGATATCACCAACACAACCAGTGGATGGAACAAAGCCGGGCTTATTGCTTTCGGCAGTAAGGGGGTTTGTTTGATGGTGCTATCAGAGGGGGAAAGGCAAGGCCAGATTACTGAAAATTTGCAGCTTGGTTTAAAGTCCGTTTTTGACAGGGCTTGATAAGGATTGGGTTAGGTGTCGTGATATGATGTTACAGGATTGTGGGAACAAAGTCCTAGGGCATAAACTGTTGGTGCTTCCTATTGAAGTGTTAACGGGTCTTTTGGGAAGTTTCCATAATGAGCAATTCATTTATTTGTGCAGGCAAGAATAAAAGTAAAGACAATGGAAACATGTAGACAGTTCTAACTGTGGAGGTTCTGGAGGGTGTGGAAGTTCTGTTCTCACCTCTGAGTAGAGGAATTGGGAGACTGGAGGACAAAATAAGAGGAAGATTTATTTTTCACTGTTTGTCCTTTTACACTCTTAACATTTTAAAAAGCACATCTCTGTATAGCCCATTCCAAAAAGATAATTATGCATTTTTTAATGCATGTGTATTTAGTGTTTTACTTCATCATAGAGCCTTGTTTATTCTATTCAGATAGAAACAATTGTTTATCAAATAAAATTGTCCTCCAGAAAAACA

SEQ ID NO: 28 - protein, 343 aa, Homo sapiens
MALTPTNLNNKMSLQMKMDCQEQQLTKKNNGFFQKLNVTEGAMQDLLKEIIKVDHILDRSDDEDDISSENPQTDFLHKGMLELEAEHDQDLSKQDKQETDVDEDPQASTSLQFSKKNLLELCLKGMFLKLNYWNTKIGLQVKELGADYIDGTEKIDNIIKKINVTENTVKSLLKDMLTLKGQIEKLEDRGLDLDQGTSTEVNTCNEVYELKKKVIERLEDLCKNVELLSAKLRMYQMEAEDTDSHSSEEIDTEEMEALLPQAPASFLVQKSPPRNTAWKRALRIFIMFDVLTVTGLLCYILFFGATFLFERVLLRMLGCRTTWDLREMREPFLNLEVEALLPS

SEQ ID NO: 29 - protein, 116 aa, Homo sapiens
MKGLRSLAATTLALFLVFVFLGNSSCAPQRLLERRNWTPQAMLYLKGAQGRRFISDQSRRKDLSDRPLPERRSPNPQLLTIPEAATILLASLQKSPEDEEKNFDQTRFLEDSLLNW

SEQ ID NO: 30 - protein, 622 aa, Homo sapiens
MASPLPSGFPARRNSRLDVFLRRHLPPEVYDAVRAYEPCIVVSNSENHILKYVVLSDRLVYLTENPPKSIRRVVALRDVVAIDLIDDYPEFLSSPDREISQHIRIIYSSTVLKKECKKSNSVRKFLFPFHHTKANNKKVKEEKNGLAFWRSKESRSLKESPLRDQQESSTPSKDSTLCPRPGLKKLSLHGQGAFRPLPSPSRRSSQSAPTTGKAVSEPSCTTNTKEPQGLPDHNSISEIPFKCNGNGNEFYLGNSLLDSPSQSNSNLEKKESELHLYVISTTSSIFLHLKSSWNNYIIKATLLQDPFYASEFSPAIGSQKPYRSEEKIKHFSQLKSELFLKDNSLRRILSLLMELKVAAQKNFILKRLFWKTSDLFYFIVNKLHEYLPESRDKNALQNQSQRVDELVACIEIIQTLVLMFRETETESSRLNTLAAKKGALFNLLVILISEPQIPKSCPVFDIQLVADSALVRMSFDAELQKLILEYTNTATALLYEILLVFQQGNLGLGSTKFAISWIMSFLQSCPPIITFVASIVKQVVRGLSASFQLLSPCQAVLLYQQFYILKSCLRHSRTLAEYIRNNYREEFRYFIHMPALQKRLPLCYPITQPTIQLFHEVLKLVE

SEQ ID NO: 31 - protein, 83 aa, Homo sapiens
MSFFQLLMKRKELIPLVVFMTVAAGGASSFAVYSLWKTDVILDRKKNPEPWETVDPTVPQKLITINQQWKPIEELQNVQRVTK

SEQ ID NO: 32 - protein, 249 aa, Homo sapiens
MLQLWKLVLLCGVLTGTSESLLDNLGNDLSNVVDKLEPVLHEGLETVDNTLKGILEKLKVDLGVLQKSSAWQLAKQKAQEAEKLLNNVISKLLPTNTDIFGLKISNSLILDVKAEPIDDGKGLNLSFPVTANVTVAGPIIGQIINLKASLDLLTAVTIETDPQTHQPVAVLGECASDPTSISLSLLDKHSQIINKFVNSVINTLKSTVSSLLQKEICPLIRIFIHSLDVNVIQQVVDNPQHKTQLQTLI

SEQ ID NO: 33 - protein, 261 aa, Homo sapiens
MAESHLQSSLITASQFFEIWLHFDADGSGYLEGKELQNLIQELQQARKKAGLELSPEMKTFVDQYGQRDDGKIGIVELAHVLPTEENFLLLFRCQQLKSCEEFMKTWRKYDTDHSGFIETEELKNFLKDLLEKANKTVDDTKLAEYTDLMLKLFDSNNDGKLELTEMARLLPVQENFLLKFQGIKMCGKEFNKAFELYDQDGNGYIDENELDALLKDLCEKNKQDLDINNITTYKKNIMALSDGGKLYRTDLALILCAGDN

SEQ ID NO: 34 - protein, 141 aa, Homo sapiens
MGFQKFSPFLALSILVLLQAGSLHAAPFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 35 - protein, 198 aa, Homo sapiens
MADDQGCIEEQGVEDSANEDSVDAKPDRSSFVPSLFSKKKKNVTMRSIKTTRDRVPTYQYNMNFEKLGKCIIINNKNFDKVTGMGVRNGTDKDAEALFKCFRSLGFDVIVYNDCSCAKMQDLLKKASEEDHTNAACFACILLSHGEENVIYGKDGVTPIKDLTAHFRGDRCKTLLEKPKLFFIQACRGTELDDGIQAD

SEQ ID NO: 36 - protein, 780 aa, Homo sapiens
MDQNSVPEKAQNEADTNNADRFFRSHSSPPHHRPGHSRALHHYELHHHGVPHQRGESHHPPEFQDFHDQALSSHVHQSHHHSEARNHGRAHGPTGFGLAPSQGAVPSHRSYGEDYHDELQRDGRRHHDGSQYGGFHQQSDSHYHRGSHHGRPQYLGENLSHYSSGVPHHGEASHHGGSYLPHGPNPYSESFHHSEASHLSGLQHDESQHHQVPHRGWPHHHQVHHHGRSRHHEAHQHGKSPHHGETISPHSSVGSYQRGISDYHSEYHQGDHHPSEYHHGDHPHHTQHHYHQTHRHRDYHQHQDHHGAYHSSYLHGDYVQSTSQLSIPHTSRSLIHDAPGPAASRTGVFPYHVAHPRGSAHSMTRSSSTIRSRVTQMSKKVHTQDISTKHSEDWGKEEGQFQKRKTGRLQRTRKKGHSTNLFQWLWEKLTFLIQGFREMIRNLTQSLAFETFIFFVVCLNTVMLVAQTFAEVEIRGEWYFMALDSIFFCIYVVEALLKIIALGLSYFFDFWNNLDFFIMAMAVLDFLLMQTHSFAIYHQSLFRILKVFKSLRALRAIRVLRRLSFLTSVQEVTGTLGQSLPSIAAILILMFTCLFLFSAVLRALFRKSDPKRFQNIFTTIFTLFTLLTLDDWSLIYMDSRAQGAWYIIPILVIYIIIQYFIFLNLVITVLVDSFQTALFKGLEKAKQERAARIQEKLLEDSLTELRAAEPKEVASEGTMLKRLIEKKFGTMTEKQQELLFHYLQLVASVEQEQQKFRSQAAVIDEIVDTTFEAGEEDFRN

SEQ ID NO: 37 - protein, 147 aa, Homo sapiens
MADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

SEQ ID NO: 38 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 39 - protein, 290 aa, Homo sapiens; identical to SEQ ID NO: 11

SEQ ID NO: 40 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 41 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 42 - protein, 146 aa, Homo sapiens [sequence omitted]

SEQ ID NO: 43 - protein, 476 aa, Homo sapiens
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYSSSQPEPRTGLSLWDTSPSYIDKLVQGISFSQPTCPDHMLLNSQLLGTPGSSQNPWQRLVKRMTRFFTKLDADKSYQCLKETCEKLGYQWKKSCMNQVTISTTDRRNNKLIFKVNLLEMDDKILVDFRLSKGDGLEFKRHFLKIKGKLIDIVSSQKIWLPAT

SEQ ID NO: 44 - protein, 1500 aa, Homo sapiens
MTRILTAFKVVRTLKTGFGFTNVTAHQKWKFSRPGIRLLSVKAQTAHIVLEDGTKMKGYSFGHPSSVAGEVVFNTGLGGYPEAITDPAYKGQILTMANPIIGNGGAPDTTALDELGLSKYLESNGIKVSGLLVLDYSKDYNHWLATKSLGQWLQEEKVPAIYGVDTRMLTKIIRDKGTMLGKIEFEGQPVDFVDPNKQNLIAEVSTKDVKVYGKGNPTKVVAVDCGIKNNVIRLLVKRGAEVHLVPWNHDFTKMEYDGILIAGGPGNPALAEPLIQNVRKILESDRKEPLFGISTGNLITGLAAGAKTYKMSMANRGQNQPVLNITNKQAFITAQNHGYALDNTLPAGWKPLFVNVNDQTNEGIMHESKPFFAVQFHPEVTPGPIDTEYLFDSFFSLIKKGKATTITSVLPKPALVASRVEVSKVLILGSGGLSIGQAGEFDYSGSQAVKAMKEENVKTVLMNPNIASVQTNEVGLKQADTVYFLPITPQFVTEVIKAEQPDGLILGMGGQTALNCGVELFKRGVLKEYGVKVLGTSVESIMATEDRQLFSDKLNEINEKIAPSFAVESIEDALKAADTIGYPVMIRSAYALGGLGSGICPNRETLMDLSTKAFAMTNQILVEKSVTGWKEIEYEVVRDADDNCVTVCNMENVDAMGVHTGDSVVVAPAQTLSNAEFQMLRRTSINVVRHLGIVGECNIQFALHPTSMEYCIIEVNARLSRSSALASKATGYPLAFIAAKIALGIPLPEIKNVVSGKTSACFEPSLDYMVTKIPRWDLDRFHGTSSRIGSSMKSVGEVMAIGRTFEESFQKALRMCHPSIEGFTPRLPMNKEWPSNLDLRKELSEPSSTRIYAIAKAIDDNMSLDEIEKLTYIDKWFLYKMRDILNMEKTLKGLNSESMTEETLKRAKEIGFSDKQISKCLGLTEAQTRELRLKKNIHPWVKQIDTLAAEYPSVTNYLYVTYNGQEHDVNFDDHGMMVLGCGPYHIGSSVEFDWCAVSSIRTLRQLGKKTVVVNCNPETVSTDFDECDKLYFEELSLERILDIYHQEACGGCIISVGGQIPNNLAVPLYKNGVKIMGTSPLQIDRAEDRSIFSAVLDELKVAQAPWKAVNTLNEALEFAKSVDYPCLLRPSYVLSGSAMNVVFSEDEMKKFLEEATRVSQEHPVVLTKFVEGAREVEMDAVGKDGRVISHAISEHVEDAGVHSGDATLMLPTQTISQGAIEKVKDATRKIAKAFAISGPFNVQFLVKGNDVLVIECNLRASRSFPFVSKTLGVDFIDVATKVMIGENVDEKHLPTLDHPIIPADYVAIKAPMFSWPRLRDADPILRCEMASTGEVACFGEGIHTAFLKAMLSTGFKIPQKGILIGIQQSFRPRFLGVAEQLHNEGFKLFATEATSDWLNANNVPATPVAWPSQEGQNPSLSSIRKLIRDGSIDLVINLPNNNTKFVHDNYVIRRTAVDSGIPLLTNFQVTKLFAEAVQKSRKVDSKSLFHYRQYSAGKAA

SEQ ID NO: 45 - protein, 127 aa, Homo sapiens
MWMGLIQLVEGVKRKDQGFLEKEFYHKTNIKMRCEFLACWPAFTVLGEAWRDQVDWSRLLRDTGLVKMSRKPRASSPLSNNHPPTPKRRGSGRHPLNPGPEALSKFPRQPGREKGPIKEVPGTKGSP

SEQ ID NO: 46 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 47 - protein, 433 aa, Homo sapiens
MALRVTRNSKINAENKAKINMAGAKRVPTAPAATSKPGLRPRTALGDIGNKVSEQLQAKMPMKKEAKPSATGKVIDKKLPKPLEKVPMLVPVPVSEPVPEPEPEPEPEPVKEEKLSPEPILVDTASPSPMETSGCAPAEEDLCQAFSDVILAVNDVDAEDGADPNLCSEYVKDIYAYLRQLEEEQAVRPKYLLGREVTGNMRAILIDWLVQVQMKFRLLQETMYMTVSIIDRFMQNNCVPKKMLQLVGVTAMFIASKYEEMYPPEIGDFAFVTDNTYTKHQIRQMEMKILRALNFGLGRPLPLHFLRRASKIGEVDVEQHTLAKYLMELTMLDYDMVHFPPSQIAAGAFCLALKILDNGEWTPTLQHYLSYTEESLLPVMQHLAKNVVMVNQGLTKHMTVKNKYATSKHAKISTLPQLNSALVQDLAKAVAKV

SEQ ID NO: 48 - protein, 775 aa, Homo sapiens
MASRAVVRARRCPQCPQVRAAAAAPAWAALPLSRSLPPCSNSSSFSMPLFLLLLLVLLLLLEDAGAQQGDGCGHTVLGPESGTLTSINYPQTYPNSTVCEWEIRVKMGERVRIKFGDFDIEDSDSCHFNYLRIYNGIGVSRTEIGKYCGLGLQMNHSIESKGNEITLLFMSGIHVSGRGFLASYSVIDKQDLITCLDTASNFLEPEFSKYCPAGCLLPFAEISGTIPHGYRDSSPLCMAGVHAGVVSNTLGGQISVVISKGIPYYESSLANNVTSVVGHLSTSLFTFKTSGCYGTLGMESGVIADPQITASSVLEWTDHTGQENSWKPKKARLKKPGPPWAAFATDEYQWLQIDLNKEKKITGIITTGSTMVEHNYYVSAYRILYSDDGQKWTVYREPGVEQDKIFQGNKDYHQDVRNNFLPPIIARFIRVNPTQWQQKIAMKMELLGCQFIPKGRPPKLTQPPPPRNSNDLKNTTAPPKIAKGRAPKFTQPLQPRSSNEFPAQTEQTTASPDIRNTTVTPNVTKDVALAAVLVPVLVMVLTTLILILVCAWHWRNRKKKTEGTYDLPYWDRAGWWKGMKQFLPAKAVDHEETPVRYSSSEVNHLSPREVTTVLQADSAEYAQPLVGGIVGTLHQRSTFKPEEGKEAGYADLDPYNSPGQEVYHAYAEPLPITGPEYATPIIMDMSGHPTTSVGQPSTSTFKATGNQPPPLVGTYNTLLSRTDSCSSAQAQYDTPKAGKPGLPAPDELVYQVPQSTQEVSGAGRDGECDVFKEIL

SEQ ID NO: 49 - protein, 189 aa, Homo sapiens
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 50 - protein, 394 aa, Homo sapiens
MVTMEELREMDCSVLKRLMNRDENGGGAGGSGSHGTLGLPSGGKCLLLDCRPFLAHSAGYILGSVNVRCNTIVRRRAKGSVSLEQILPAEEEVRARLRSGLYSAVIVYDERSPRAESLREDSTVSLVVQALRRNAERTDICLLKGGYERFSSEYPEFCSKTKALAAIPPPVPPSATEPLDLGCSSCGTPLHDQGGPVEILPFLYLGSAYHAARRDMLDALGITALLNVSSDCPNHFEGHYQYKCIPVEDNHKADISSWFMEAIEYIDAVKDCRGRVLVHCQAGISRSATICLAYLMMKKRVRLEEAFEFVKQRRSIISPNFSFMGQLLQFESQVLATSCAAEAASPSGPLRERGKTPATPTSQFVFSFPVSVGVHSAPSSLPYLHSPITTSPSC

SEQ ID NO: 51 - protein, 595 aa, Homo sapiens [sequence omitted]

SEQ ID NO: 52 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 53 - protein, 466 aa, Homo sapiens
MVSQALRLLCLLLGLQGCLAAGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 54 - protein, 355 aa, Homo sapiens
MPFLGQDWRSPGQNWVKTADGWKRFLDEKSGSFVSDLSSYCNKEVYNKENLFNSLNYDVAAKKRKKDMLNSKTKTQYFHQEKWIYVHKGSTKERHGYCTLGEAFNRLDFSTAILDSRRFNYVVRLLELIAKSQLTSLSGIAQKNFMNILEKVVLKVLEDQQNIRLIRELLQTLYTSLCTLVQRVGKSVLVGNINMWVYRMETILHWQQQLNNIQITRPAFKGLTFTDLPLCLQLNIMQRLSDGRDLVSLGQAAPDLHVLSEDRLLWKKLCQYHFSERQIRKRLILSDKGQLDWKKMYFKLVRCYPRKEQYGDTLQLCKHCHILSWKGTDHPCTANNPESCSVSLSPQDFINLFKF

SEQ ID NO: 55 - protein, 801 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKPLDPGSPQLPEHLESQQKRPNPELRRNMTIKTELPLGARRKMKPLLPRVSSYLVPIQFPVNQSLVLQPSVKVPLPLAASLMSSELARHSKRVRIAPKVFGEQVVFGYMSKFFSGDLRDFGTPITSLFNFIFLCLSVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 56 - protein, 444 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 57 - protein, 595 aa, Homo sapiens
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 58 - protein, 592 aa, Homo sapiens
MASEIHMTGPMCLIENTNGRLMANPEALKILSAITQPMVVVAIVGLYRTGKSYLMNKLAGKKKGFSLGSTVQSHTKGIWMWCVPHPKKPGHILVLLDTEGLGDVEKGDNQNDSWIFALAVLLSSTFVYNSIGTINQQAMDQLYYVTELTHRIRSKSSPDENENEVEDSADFVSFFPDFVWTLRDFSLDLEADGQPLTPDEYLTYSLKLKKGTSQKDETFNLPRLCIRKFFPKKKCFVFDRPVHRRKLAQLEKLQDEELDPEFVQQVADFCSYIFSNSKTKTLSGGIQVNGPRLESLVLTYVNAISSGDLPCMENAVLALAQIENSAAVQKAIAHYEQQMGQKVQLPTETLQELLDLHRDSEREAIEVFIRSSFKDVDHLFQKELAAQLEKKRDDFCKQNQEASSDRCSALLQVIFSPLEEEVKAGIYSKPGGYRLFVQKLQDLKKKYYEEPRKGIQAEEILQTYLKSKESMTDAILQTDQTLTEKEKEIEVERVKAESAQASAKMLQEMQRKNEQMMEQKERSYQEHLKQLTEKMENDRVQLLKEQERTLALKLQEQEQLLKEGFQKESRIMKNEIQDLQTKMRRRKACTIS

SEQ ID NO: 59 - protein, 1020 aa, Homo sapiens
MQSCARAWGLRLGRGVGGGRRLAGGSGPCWAPRSRDSSSGGGDSAAAGASRLLERLLPRHDDFARRHIGPGDKDQREMLQTLGLASIDELIEKTVPANIRLKRPLKMEDPVCENEILATLHAISSKNQIWRSYIGMGYYNCSVPQTILRNLLENSGWITQYTPYQPEVSQGRLESLLNYQTMVCDITGLDMANASLLDEGTAAAEALQLCYRHNKRRKFLVDPRCHPQTIAVVQTRAKYTGVLTELKLPCEMDFSGKDVSGVLFQYPDTEGKVEDFTELVERAHQSGSLACCATDLLALCILRPPGEFGVDIALGSSQRFGVPLGYGGPHAAFFAVRESLVRMMPGRMVGVTRDATGKEVYRLALQTREQHIRRDKATSNICTAQALLANMAAMFAIYHGSHGLEHIARRVHNATLILSEGLKRAGHQLQHDLFFDTLKIQCGCSVKEVLGRAAQRQINFRLFEDGTLGISLDETVNEKDLDDLLWIFGCESSAELVAESMGEECRGIPGSVFKRTSPFLTHQVFNSYHSETNIVRYMKKLENKDISLVHSMIPLGSCTMKLNSSSELAPITWKEFANIHPFVPLDQAQGYQQLFRELEKDLCELTGYDQVCFQPNSGAQGEYAGLATIRAYLNQKGEGHRTVCLIPKSAHGTNPASAHMAGMKIQPVEVDKYGNIDAVHLKAMVDKHKENLAAIMITYPSTNGVFEENISDVCDLIHQHGGQVYLDGANMNAQVGICRPGDFGSDVSHLNLHKTFCIPHGGGGPGMGPIGVKKHLAPFLPNHPVISLKRNEDACPVGTVSAAPWGSSSILPISWAYIKMMGGKGLKQATETAILNANYMAKRLETHYRILFRGARGYVGHEFILDTRPFKKSANIEAVDVAKRLQDYGFHAPTMSWPVAGTLMVEPTESEDKAELDRFCDAMISIRQEIADIEEGRIDPRVNPLKMSPHSLTCVTSSHWDRPYSREVAAFPLPFVKPENKFWPTIARIDDIYGDQHLVCTCPPMEVYESPFSEQKRASS

SEQ ID NO: 60 - protein, 75 aa, Homo sapiens
MKEGMSNNSTTSISQARKAVEQLKMEACMDRVKVSQAAADLLAYCEAHVREDPLIIPVPASENPFREKKFFCTIL

SEQ ID NO: 61 - protein, 396 aa, Homo sapiens
MWNSSDANFSCYHESVLGYRYVAVSWGVVVAVTGTVGNVLTLLALAIQPKLRTRFNLLIANLTLADLLYCTLLQPFSVDTYLHLHWRTGATFCRVFGLLLFASNSVSILTLCLIALGRYLLIAHPKLFPQVFSAKGIVLALVSTWVVGVASFAPLWPIYILVPVVCTCSFDRIRGRPYTTILMGIYFVLGLSSVGIFYCLIHRQVKRAAQALDQYKLRQASIHSNHVARTDEAMPGRFQELDSRLASGGPSEGISSEPVSAATTQTLEGDSSEVGDQINSKRAKQMAEKSPPEASAKAQPIKGARRAPDSSSEFGKVTRMCFAVFLCFALSYIPFLLLNILDARVQAPRVVHMLAANLTWLNGCINPVLYAAMNRQFRQAYGSILKRGPRSFHRLH

SEQ ID NO: 62 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 63 - protein, 462 aa, Homo sapiens
MGQDAFMEPFGDTLGVFQCKIYLLLFGACSGLKVTVPSHTVHGVRGQALYLPVHYGFHTPASDIQIIWLFERPHTMPKYLLGSVNKSVVPDLEYQHKFTMMPPNASLLINPLQFPDEGNYIVKVNIQGNGTLSASQKIQVTVDDPVTKPVVQIHPPSGAVEYVGNMTLTCHVEGGTRLAYQWLKNGRPVHTSSTYSFSPQNNTLHIAPVTKEDIGNYSCLVRNPVSEMESDIIMPIIYYGPYGLQVNSDKGLKVGEVFTVDLGEAILFDCSADSHPPNTYSWIRRTDNTTYIIKHGPRLEVASEKVAQKTMDYVCCAYNNITGRQDETHFTVIITSVGLEKLAQKGKSLSPLASITGISLFLIISMCLLFLWKKYQPYKVIKQKLEGRPETEYRKAQTFSGHEDALDDFGIYEFVAFPDVSGVSRIPSRSVPASDCVSGQDLHSTVYEVIQHIPAQQQDHPE

SEQ ID NO: 64 - protein, 338 aa, Homo sapiens
MNDFDECGQSAASMYLPGCAYYVAPSDFASKPSFLSQPSSCQMTFPYSSNLAPHVQPVREVAFRDYGLERAKWPYRGGGGGGSAGGGSSGGGPGGGGGGAGGYAPYYAAAAAAAAAAAAAEEAAMQRELLPPAGRRPDVLFKAPEPVCAAPGPPHGPAGAASNFYSAVGRNGILPQGFDQFYEAAPGPPFAGPQPPPPPAPPQPEGAADKGDPRTGAGGGGGSPCTKATPGSEPKGAAEGSGGDGEGPPGEAGAEKSSSAVAPQRSRKKRCPYTKYQIRELEREFFFNVYINKEKRLQLSRMLNLTDRQVKIWFQNRRMKEKKLNRDRLQYFTGNPLF

SEQ ID NO: 65 - protein, 343 aa, Homo sapiens
MSRAGSWDMDGLRADGGGAGGAPASSSSSSVAAAAASGQCRGFLSAPVFAGTHSGRAAAAAAAAAAAAAAASGFAYPGTSERTGSSSSSSSSAVVAARPEAPPAKECPAPTPAAAAAAPPSAPALGYGYHFGNGYYSCRMSHGVGLQQNALKSSPHASLGGFPVEKYMDVSGLASSSVPANEVPARAKEVSFYQGYTSPYQHVPGYIDMVSTFGSGEPRHEAYISMEGYQSWTLANGWNSQVYCTKDQPQGSHFWKSSFPGDVALNQPDMCVYRRGRKKRVPYTKLQLKELENEYAINKFINKDKRRRISAATNLSERQVTIWFQNRRVKDKKIVSKLKDTVS

SEQ ID NO: 66 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 67 - protein, 577 aa, Homo sapiens
MNKLYIGNLNESVTPADLEKVFAEHKISYSGQFLVKSGYAFVDCPDEHWAMKAIETFSGKVELQGKRLEIEHSVPKKQRSRKIQIRNIPPQLRWEVLDSLLAQYGTVENCEQVNTESETAVVNVTYSNREQTRQAIMKLNGHQLENHALKVSYIPDEQIAQGPENGRRGGFGSRGQPRQGSPVAAGAPAKQQQVDIPLRLLVPTQYVGAIIGKEGATIRNITKQTQSKIDVHRKENAGAAEKAISVHSTPEGCSSACKMILEIMHKEAKDTKTADEVPLKILAHNNFVGRLIGKEGRNLKKVEQDTETKITISSLQDLTLYNPERTITVKGAIENCCRAEQEIMKKVREAYENDVAAMSLQSHLIPGLNLAAVGLFPASSSAVPPPPSSVTGAAPYSSFMQAPEQEMVQVFIPAQAVGAIIGKKGQHIKQLSRFASASIKIAPPETPDSKVRMVIITGPPEAQFKAQGRIYGKLKEENFFGPKEEVKLETHIRVPASAAGRVIGKGGKTVNELQNLTAAEVVVPRDQTPDENDQVIVKIIGHFYASQMAQRKIRDILAQVKQQHQKGQSNQAQARRK

SEQ ID NO: 68 - protein, 139 aa, Homo sapiens
MASLFRSYLPAIWLLLSQLLRESLAAELRGCGPRFGKHLLSYCPMPEKTFTTTPGGWLLESGRPKEMVSTSNNKDGQALGTTSEFIPNLSPELKKPLSEGQPSLKKIILSRKKRSGRHRFDPFCCEVICDDGTSVKLCT

SEQ ID NO: 69 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 70 - protein, 242 aa, Homo sapiens
MSDSKCDSQFYSVQVADSTFTVLKRYQQLKPIGSGAQGIVCAAFDTVLGINVAVKKLSRPFQNQTHAKRAYRELVLLKCVNHKNIISLLNVFTPQKTLEEFQDVYLVMELMDANLCQVIHMELDHERMSYLLYQMLCGIKHLHSAGIIHRDLKPSNIVVKSDCTLKILDFGLARTACTNFMMTPYVVTRYYRAPEVILGMGYKENVDIWSVGCIMGELVKGCVIFQGTDRILPRDLGPAMLS

SEQ ID NO: 71 - protein, 430 aa, Homo sapiens
MSSRSPRPPPRRSRRRLPRPSCCCCCCRRSHLNEDTGRFVLLAALIGLYLVAGATVFSALESPGEAEARARWGATLRNFSAAHGVAEPELRAFLRHYEAALAAGVRADALRPRWDFPGAFYFVGTVVSTIGFGMTTPATVGGKAFLIAYGLFGCAGTILFFNLFLERIISLLAFIMRACRERQLRRSGLLPATFRRGSALSEADSLAGWKPSVYHVLLILGLFAVLLSCCASAMYTSVEGWDYVDSLYFCFVTFSTIGFGDLVSSQHAAYRNQGLYRLGNFLFILLGVCCIYSLFNVISILIKQVLNWMLRKLSCRCCARCCPAPGAPLARRNAITPGSRLRRRLAALGADPAARDSDAEGRRLSGELISMRDLTASNKVSLALLQKQLSETANGYPRSVCVNTRQNGFSGGVGALGIMNNRLAETSASR

SEQ ID NO: 72 - protein, 1149 aa, Homo sapiens
MFQTKLRNETWEDLPKMSCTTEIQAAFILSSFVTFFSGLIILLIFRLIWRSVKKWQIIKGTGIILELFTSGTIARSHVRSLHFQGQFRDHIEMLLSAQTFVGQVLVILVFVLSIGSLIIYFINSADPVGSCSSYEDKTIPIDLVFNAFFSFYFGLRFMAADDKIKFWLEMNSIVDIFTIPPTFISYYLKSNWLGLRFLRALRLLELPQILQILRAIKTSNSVKFSKLLSIILSTWFTAAGFIHLVENSGDPWLKGRNSQNISYFESIYLVMATTSTVGFGDVVAKTSLGRTFIMFFTLGSLILFANYIPEMVELFANKRKYTSSYEALKGKKFIVVCGNITVDSVTAFLRNFLRDKSGEINTEIVFLGETPPSLELETIFKCYLAYTTFISGSAMKWEDLRRVAVESAEACLIIANPLCSDSHAEDISNIMRVLSIKNYDSTTRIIIQILQSHNKVYLPKIPSWNWDTGDNIICFAELKLGFIAQGCLVPGLCTFLTSLFVEQNKKVMPKQTWKKHFLNSMKNKILTQRLSDDFAGMSFPEVARLCFLKMHLLLIAIEYKSLFTDGFCGLILNPPPQVRIRKNTLGFFIAETPKDVRRALFYCSVCHDDVFIPELITNCGCKSRSRQHITVPSVKRMKKCLKGISSRISGQDSPPRVSASTSSISNFTTRTLQHDVEQDSDQLDSSGMFHWCKPTSLDKVTLKRTGKSKYKFRNHIVACVFGDAHSAPMGLRNFVMPLRASNYTRKELKDIVFIGSLDYLQREWRFLWNFPQIYILPGCALYSGDLHAANIEQCSMCAVLSPPPQPSSNQTLVDTEAIMATLTIGSLQIDSSSDPSPSVSEETPGYTNGHNEKSNCRKVPILTELKNPSNIHFIEQLGGLEGSLQETNLHLSTAFSTGTVFSGSFLDSLLATAFYNYHVLELLQMLVTGGVSSQLEQHLDKDKVYGVADSCTSLLSGRNRCKLGLLSLHETILSDVNPRNTFGQLFCGSLDLFGILCVGLYRIIDEEELNPENKRFVITRPANEFKLLPSDLVFCAIPFSTACYKRNEEFSLQKSYEIVNKASQTTETHSDTNCPPTIDSVTETLYSPVYSYQPRTNSLSFPKQIAWNQSRTNSIISSQIPLGDNAKENERKTSDEVYDEDPFAYSEPL

SEQ ID NO: 73 - protein, 251 aa, Homo sapiens
MFLLLTALQVLAIAMTQSQEDENKIIGGHTCTRSSQPWQAALLAGPRRRFLCGGALLSGQWVITAAHCGRPILQVALGKHNLRRWEATQQVLRVVRQVTHPNYNSRTHDNDLMLLQLQQPARIGRAVRPIEVTQACASPGTSCRVSGWGTISSPIARYPASLQCVNINISPDEVCQKAYPRTITPGMVCAGVPQGGKDSCQGDSGGPLVCRGQLQGLVSWGMERCALPGYPGVYTNLCKYRSWIEETMRDK

SEQ ID NO: 74 - protein, 509 aa, Homo sapiens
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 75 - protein, 234 aa, Homo sapiens
MTSVASLPEQSPGYRCRSLTGAGARTSAAGPLARLLPRRRLPPPHLPARPPGGWSRNQSHQRRGPGRCWGWGRGMTRICPLCPGAPEYINSAVSSQTLSQSCTPPQSPQPDPRPLSLRVGLALPGGRLGCGEAGSANRHWFSFLFPFIHFCWSPRAAAPGVAMNGWMPARWDHQVRRDVAGARGAPPAWGQAPSPRRSVGGPQKTLKRPKACSPRSPQHTPGVFSAPEKLGRKT

SEQ ID NO: 76 - DNA, 8531 bp, Homo sapiens
  source 1..8531: mol_type = other DNA
agccctgcgcttccccaggtgaaccgggcaggagcctgttgggaaggcagcgacccacatctgtgtgcacctttgtggatttcaggttccggacgcaggcgaccaagccagagccagtaagcagccttgcctccaggataataccgcttatcaggcttgactctgtgcgcctctgagccaggggtgtgcccagcagctgggagcagactctgcggtcggagcgtggacgcgttctgtgcagctctgcgccctggggtcaggctgtgctcgcctccctctgccgctgcttccccattccagagggatgggtagatattaggggtattttgaggattcagagagaatgcatgtagaatgcctagctcagggtcagcccttagaaccaacatattttagcgactattaatcttcatgacaactctcattcctcttgtgtcccctactttacagataaacaaactgagtcttcctgagattaaatataatagagataatagataatagatctagataatagatcgcctaaactcacgctattgggtaaatgaaagcctaggttggtccccaacactaaatgcttttttctcaaaggatggtctctttaacttccttctttgggcttatattcagacagttttcttctttaggttgaatttgttctgtgatccctacattgctcaccctggagactgacaggagaatgaaaattccatgagtagggaatagttttgctagaaaatgtttaataacaagttccaggctgggggtgggatgtgcatatgatatatacatctgtgtgttgcatacctacatctatatacacatatataataaattttactaatataaaaactgtgtagtacacaaattacaaacagtaaaaagtatacaatcatatttagtatacattccatgagagacagttaatcttcatggaatgcttctggttatgtttgccaaactcgtgaatctttagccaatctcagttgcaatcggtgcaaaagtgttgttccaaattgaactatagttagtatttgcatttgatcagagggcttttcacagcctgcaaggaggctttttttggcaattagttcatcatctgtgcccacccttcagaatagaagcacctcaagagcaggcacccagtgatcagtgtgaatttctagtgccaaaggttactggtgcaggacccctggttgtttagatgcctgggtctgtgctggctctgcctcttattatattgggaaacttgggcaatttattaactttctgtgtctcaattttctcatctgcaagtgagactaatagtccctatctggcagggttgctgggaggattaactgagttgatctgagaccagtgatgataatggaggctggcatatagaagtgctcagtaaatgtcagcgattgtcattttattgtaaagaaactctgcatttaaaaaagttaaattgacattcatagaacatagttaatcattttaaagcgaacagctcagtggcatttagtgcatttacaatgttgtacagccccctcctctacctatttccaaaacattctcattaccccgaaaggagactctgtacccgctagtggtccttctccatttctccctgttctctgtccctggcaacaatgaatccacttcctgccgctgtggctttatttaattcaggacatttcatatcaatggcattatactccatgtggccttttgtgactggcttctttcacttagcgtggtgttttcaaggcccctacatcttgtagtacatatcagagcctcattcctttatacagctgaataatcttctactttacggatatatcacaattgtttatccattcatccattgtttctacttttttaaaagctttttaattgatgcataataggtgtacatagttttgagacatatgtggtaatttgatacattcaaataatttgtaaagatcaaatctatgtacttgggatctccatcatcttaaatattgtcttcatgctggtaccattctatttcttctcttctagctattttgaaatatgcaagagattattggaaactgtagtcacgctactgatctaccaaacactaggtcccatttcttctatcaaactccatattcgtacccactaatccatcattttactattgtgaatagtgttgctatgaatgcgttgtgaatatgttgtgtttgagtatctgttttccattctgtttgatctaagcctaggattggaactgctgggtcatgtggtaattctacattgaactttttgaggaaccaccaaactgctgttcacagtggctgcactattgtccactcccaccggtcgtgcaggagggccccactttctccacatccttcttgatacttgttgttctccgcctttttgagtagcgtcatcctaggagatgtgaagtggtatctcattgtggttttgatttgcatttctctcgtgacttaatggtgttgagcactgaaatggtttgtccgtgtccccacccaaatcttgtcttgaattgcaatcccataatacccatgtgttgtgggagggacccagtgggaggtaatttaatcacgggggcagttacccccatgctgcttttcttgtgatagtgagtgagttctcacaagatctgatggttttataaggggctttcccctcttttgttcggcacttctccttgctgctgccatgtgaagaaggatgtgtttgcttctgcttccaccatgatcgtaagtttcctgaggcctccccagccctggggaactgtgagtcaattaaacctctttcctttataaattatccagtatccggtatgtctttattagcagtgtgagaacagactaatacaagcatctttgcctgtgattattggccatttgtatatcttctttggagaaatgtctgctcaagtcatttgaccacttttttttcccactggattgtctttttgtgcagtgcattctcgtggagtgaatggactgcctttgtcctgctggtctgcatgtgaactcccaggctctgattcctcttcattcagttgtgactccaacctcctgagtccctgccaacctgaagcacccacgatgtcactggcctctatggcactcctgaggcagggagtaggtggcaatgttatcagccctgcctggctctcactcccccacatctgggaaacaggctaatagcaccttgcttacagggctgtcattataatggaccagagtaagcccattcccagatgggaacctgagataccggctgctttggaatggctttgcgtcccagtgacacccattaaatccttaatatttctaagagctccgagatgctaagatgaaaggaatggtgtcaggcaagcatgttattatctggattattacttcccgagggaggacacttcctcattttcttccatgcaggtgggacaggtatagagagatgcccaatttgaaggctgatttgtctcttcagttctcatctagaaagactttttttttttttcctgatccaaaccatcaaagctccctagcaggattgaggaggtctgagaagctggggtcactcaccagtttacatgaaaaccgacctgagaagctgggatcgttcactagtttacatgaaaacagacctggaggccctcagctgctgggagcccaggctcctgcctgctctcggggccccagatcaacccacctcctagggcgggctctttatggagacctcacaaccactcctcattcgggctggctttgttttgattctcctgacaaccttctctttttggactttaacaaggcatatatactagtttgttgatttcataggtaaacttatgggtgtaataaccatcttctctgcatatatatttgagatggaaactagttaaggttcagtttaaaaaatattattgaacagctgctatgatcaggctctggagccagcaacagtgataagtcagacatggcttgtgttctcagaaagcttatgatctggagggaggagacacgggatacagagcaaacacatgaggacatctgtaaaaagtggtagtgatgagtgctaccaggaaacatggaacagggccggtgacaggggaacatggaggggaacaattagacggagacattggggagggctcctggaggaggagatgtgagagctggcaccaaaatcaacccaggaggccagccacggtgggatgcaggagaatggtgagatgcgggagaatggtggtatgcgggagagggattcctgggaggatggaagagccagttcacagatcctgaagcaggaacaggtttgatgattgaggctgcagacagcaggtctgtgttgtcatacacagtgagttgaagaggtagacagggcccacatcacgcagggccttgttgaagagacacgattttgctctaagatacatcagtcagcccttgctgttgttaatagaaaaactttagccaaattaaatttaaaggagattaattgagcaaagaacgattcacaaatcaggcagtctcctgagccaggctaggcccagaaactccagcacagccatggggcggaagaagatttatcaacagagaaaggaaagtgaagtacagaaaacagaagtaagggacagaaacagccagatttgtcagaggcatttgaaccagagcaactcccttttgaataggggctgggtaaaatgaggctgagacctggtgggctgcattcccaggaggttaagcattctaagtcacaggatgagatgggaagtcagcacaagatagcagtcataaagaccttgctgataaaacaggttgcagtaaagaagcggctaaaacccaccaaaaccaagatggcgatgagagtgacctctggctgtcctcactgctacactcccgccagcgccatgacagtttaggaatgccatggcaacatcaggaagttaccctatatgctctagaaaggggaggcaggaataatccaccccttgtttaacaattcgtcaagaaataaccataaaaataggcatgataagagattagttacagctctgtgtttgcatgatttggacagtttgaacagttggctacctttccctggccaaactcctcatcccagtgccctcccaggagctgcccgatgaagatgccatcagcattgggagccgtgccgggtgctctctgggccgcagaaagaaacggggcattaattgacattcttagcaagcaagtagggtctcccctaaagtggggcccatcttgaggtggggcctgcatggactgtgtccagcttgcaatgcacgtcatctcccatttctcccaggcgctgtcatacgcagagcacctgccgagccatgtgacgtgctgctctctgccagccctgatcgccttcctttccagcaggacttgcgcttcacactggaccttgaggccccatggagatctgccattttgggcaggttctgctcacagtgccagtaacttcacctaggagaagagcgacaacttcaaggccgcatgacccatctgattgtcatcacacaggtccccgcaggagaatttccatggcgtttctcactctttgtttgcaaagaatttctttttttatcgttttctttttctttttctattttcttttttcttttttttttgagacagagtctctctctgttgcccaggctggagtgcagtggcatgacctcggctcactgcaacctcggcctcctgggttcaagcaattctcctgcctcagcctcccgagtagctgggattacgggcacacaccacaacgctcagctaatttttgtatttttagaaaagacagggttttaccatgttggccaggctggtctcgatcttctgagctcgtgatccacctgcctcggcctccaaagtgctgggattacaggcgtgagccaccacacccagcctaagaatttcttagaattaatatctgcacttggccccacatcctctgagtgataacttagaactgtcactttaattgttgagagaggatattcagagaatagtgggtgattttacaagcaaggatatgtttggggagcatgttcccggtgcattctcagggtctcgtgcattcttttagacattgaagacatcctgatctccagtggaagcagagccccagtcatacacttatccaccggggacgagttccaagacccccagggaatgcctgaaagctcgggtagtgcttacctctatggatgttgtgcacaaatttatttttccctctttacaacttcatagattgaaggttcgttcttaccaaagatcttagcaaccttagcttatcattccttttatttcctgattaagtggagaactttcaccttttcactaaaaaaaaaaagtgctttacggctgcctttgccacgtgcacatggtcttttgggaccatccttaagtcaaataagggtgacctggatgcaagcactgtgatacagagaaagtccagctcaccaccaagatggctgctgagccgctggtgggcgcggagtgtcgtgtctgcagcgtgaacatgccgggcaaaggaataatgcatgtcagggatagggtggagaaggactgtgccaggcttcatcacactactcagaatggcacacagtttagaacttataaatgatttatttctggagtgatccactgcatattttcagacggtggttgatcgtgggtaactgaaagcaaaacctgactgaaaggggctgctgtgttcatgtcagcaacgctgatgccctcgaggccagaagagtaaccaccaatctggtacctgagcacctggctgtaagtccaccctccatttctggccagggccgaggaattctctctttttctctttattttcctcttgatttgagaaggtcaactctgagcatgctctaaacacaaatcagcatctctacacttcttcggcaccaggtgttgctactgtctttctgattagaaaaaaagaaatatccaggaattctggaagacagtgattgatttgcagctatgctgcaatggagctaagaatcattagcacaacccgtgctggtgttcgacaagatgcccaatcaattcatccgatgacaggtgcgctgtgtctgcgaagcgcgaagccacggagtgaggtgctgaccaggctccaccaggagcaaggaacaagactccccaggggctcctggtccacagaaggcagggacgcccacaaacataccatgagtaaatagttccaatgtgaagaagactgaaaatgccacagaagtgtgagcccactgttttgaaattctttctgctgaagggattgagaagtaagtggactcctagattttctttggaaatgccaccgaaagggataaaagtaggggaaacattattttaaatacactttttctaagcattttctccatttcttttaaaaggaactcttccataaaggagcatgaccgagccatgagttataatatatacatgcatatagaaagaggatgtgaatacagttgcagaagggcgtcaaagccaaaaacacacagggaatgacttgaggaagctgaggaccaagagcagtgctagccatgccagtgactgtggtcgctgagaactggatgatggatgtttgagtgaccgtcaggaggatcagcatgggaccaggaggatcagcatgggaccaggaggatcagcatgggaccaggaggatcagcatgggaccaggaaacagcaggcgagtgaatgtgaactccactctcaacagggagatgacgtgggtctcggaaacctacagatctgagtgcttgctgtcaggtcatggcagggctctgactgcactgtggagggaacatctgtgtttttgtcctcctggaacctagtgcccctttcctggaggcagcatcctatttgacctgtgtgggtccaaccccacctctctcctgtagcagtggcatgggacccggggctgggccattcgcagcattgccacaggcagcaatggtgattgatctaggagttgatcattgatcctaactgggtgctcaggccaaggggattgattcttggtattttttaatctcttttttccactggaattatagctaatatgatgaagcctagatatactgcgtatctggcttttagacagggcaaacctgcctagaaatgaaaatgaagtcaccacctgtgaaagcagaaacaagagcttgagagagactgagtcctggtgagaatttctgaaccccttgatccagcgatgcctgaagccagaactacttgtaaacttttcagtcatgcaatcctgtcacttctgtttgttcttaagtgcattttgtatggtttctgccgctgaccacggaaacgtccttcactaacagggaagcagagttaccccatttgagagcctcagaaggaagatggcattcatgcaaaggaagcatgcgtttgctctcacttcctgagagaaagcctgaaaatgaagccaatctcattttctttcagagatgagtctcttgatcagagaatgcctcagtt

SEQ ID NO: 77 - protein, 1390 aa, Homo sapiens; identical to SEQ ID NO: 5

SEQ ID NO: 78 - protein, 106 aa, Homo sapiens [sequence omitted]

SEQ ID NO: 79 - protein, 90 aa, Homo sapiens
MRDPVSSQYSSFLFWRMPIPELDLSELEGLGLSDTATYKVKDSSVGKMIGQATAADQEKNPEGDGLLEYSTFNFWRAPIASIHSFELDLL

SEQ ID NO: 80 - protein, 1360 aa, Homo sapiens
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 81 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 82 - protein, 752 aa, Homo sapiens
MAKRSRSEDEDDDLQYADHDYEVPQQKGLKKLWNRVKWTRDEDDKLKKLVEQHGTDDWTLIASHLQNRSDFQCQHRWQKVLNPELIKGPWTKEEDQRVIELVQKYGPKRWSLIAKHLKGRIGKQCRERWHNHLNPEVKKSSWTEEEDRIIYEAHKRLGNRWAEIAKLLPGRTDNSIKNHWNSTMRRKVEQEGYLQDGIKSERSSSKLQHKPCAAMDHMQTQNQFYIPVQIPGYQYVSPEGNCIEHVQPTSAFIQQPFIDEDPDKEKKIKELEMLLMSAENEVRRKRIPSQPGSFSSWSGSFLMDDNMSNTLNSLDEHTSEFYSMDENQPVSAQQNSPTKFLAVEANAVLSSLQTIPEFAETLELIESDPVAWSDVTSFDISDAAASPIKSTPVKLMRIQHNEGAMECQFNVSLVLEGKKNTCNGGNSEAVPLTSPNIAKFSTPPAILRKKRKMRVGHSPGSELRDGSLNDGGNMALKHTPLKTLPFSPSQFFNTCPGNEQLNIENPSFTSTPICGQKALITTPLHKETTPKDQKENVGFRTPTIRRSILGTTPRTPTPFKNALAAQEKKYGPLKIVSQPLAFLEEDIREVLKEETGTDLFLKEEDEPAYKSCKQENTASGKKVRKSLVLDNWEKEESGTQLLTEDISDMQSENRFTTSLLMIPLLEIHDNRCNLIPEKQDINSTNKTYTLTKKKPNPNTSKVVKLEKNLQSNCEWETVVYGKTEDQLIMTEQARRYLSTYTATSSTSRALIL

SEQ ID NO: 83 - protein, 420 aa, Homo sapiens
MSPPPLLQPLLLLLPLLNVEPSGATLIRIPLHRVQPGRRILNLLRGWREPAELPKLGAPSPGDKPIFVPLSNYRDVQYFGEIGLGTPPQNFTVAFDTGSSNLWVPSRRCHFFSVPCWLHHRFDPKASSSFQANGTKFAIQYGTGRVDGILSEDKLTIGGIKGASVIFGEALWEPSLVFAFAHFDGILGLGFPILSVEGVRPPMDVLVEQGLLDKPVFSFYLNRDPEEPDGGELVLGGSDPAHYIPPLTFVPVTVPAYWQIHMERVKVGPGLTLCAKGCAAILDTGTSLITGPTEEIRALHAAIGGIPLLAGEYIILCSEIPKLPAVSFLLGGVWFNLTAHDYVIQTTRNGVRLCLSGFQALDVPPPAGPFWILGDVFLGTYVAVFDRGDMKSSARVGLARARTRGADLGWGETAQAQFPG

SEQ ID NO: 84 - protein, 906 aa, Homo sapiens
MCRIAGALRTLLPLLAALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKRDWVIPPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYDIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADMYGGGDD

SEQ ID NO: 85 - protein, 165 aa, Homo sapiens
MELCRSLALLGGSLGLMFCLIALSTDFWFEAVGPTHSAHSGLWPTGHGDIISGYIHVTQTFSIMAVLWALVSVSFLVLSCFPSLFPPGHGPLVSTTAAFAAAISMVVAMAVYTSERWDQPPHPQIQTFFSWSFYLGWVSAILLLCTGALSLGAHCGGPRPGYETL

SEQ ID NO: 86 - protein, 605 aa, Homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 87 - protein, 360 aa, Homo sapiens [sequence omitted]

SEQ ID NO: 88 - protein, 680 aa, Homo sapiens
MNFETSRCATLQYCPDPYIQRFVETPAHFSWKESYYRSTMSQSTQTNEFLSPEVFQHIWDFLEQPICSVQPIDLNFVDEPSEDGATNKIEISMDCIRMQDSDLSDPMWPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSIQSPSSYGNSSPPLNKMNSMNKLPSVSQLINPQQRNALTPTTIPDGMGANIPMMGTHMPMAGDMNGLSPTQALPPPLSMPSTSHCTPPPPYPTDCSIVSFLARLGCSSCLDYFTTQGLTTIYQIEHYSMDDLASLKIPEQFRHAIWKGILDHRQLHEFSSPSHLLRTPSSASTVSVGSSETRGERVIDAVRFTLRQTISFPPRDEWNDFNFDMDARRNKQQRIKEEGE

SEQ ID NO: 89 - protein, 502 aa, Homo sapiens
MRRRRRRDGFYPAPDFRDREAEDMAGVFDIDLDQPEDAGSEDELEEGGQLNESMDHGGVGPYELGMEHCEKFEISETSVNRGPEKIRPECFELLRVLGKGGYGKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDSTLSESANQVFLGFTYVAPSVLESVKEKFSFEPKIRSPRRFIGSPRTPVSPVKFSPGDFWGRGASASTANPQTPVEYPMETSGIEQMDVTMSGEASAPLPIRQPNSGPYKKQAFPMISKRPEHLRMNL

SEQ ID NO: 90 - protein, 735 aa, Homo sapiens
MPLAQLKEPWPLMELVPLDPENGQTSGEEAGLQPSKDEGVLKEISITHHVKAGSEKADPSHFELLKVLGQGSFGKVFLVRKVTRPDSGHLYAMKVLKKATLKVRDRVRTKMERDILADVNHPFVVKLHYAFQTEGKLYLILDFLRGGDLFTRLSKEVMFTEEDVKFYLAELALGLDHLHSLGIIYRDLKPENILLDEEGHIKLTDFGLSKEAIDHEKKAYSFCGTVEYMAPEVVNRQGHSHSADWWSYGVLMFEMLTGSLPFQGKDRKETMTLILKAKLGMPQFLSTEAQSLLRALFKRNPANRLGSGPDGAEEIKRHVFYSTIDWNKLYRREIKPPFKPAVAQPDDTFYFDTEFTSRTPKDSPGIPPSAGAHQLFRGFSFVATGLMEDDGKPRAPQAPLHSVVQQLHGKNLVFSDGYVVKETIGVGSYSECKRCVHKATNMEYAVKVIDKSKRDPSEEIEILLRYGQHPNIITLKDVYDDGKHVYLVTELMRGGELLDKILRQKFFSEREASFVLHTIGKTVEYLHSQGVVHRDLKPSNILYVDESGNPECLRICDFGFAKQLRAENGLLMTPCYTANFVAPEVLKRQGYDEGCDIWSLGILLYTMLAGYTPFANGPSDTPEEILTRIGSGKFTLSGGNWNTVSETAKDLVSKMLHVDPHQRLTAKQVLQHPWVTQKDKLPQSQLSHQDLQLVKGAMAATYSALNSSKPTPQLKPIESSILAQRRVRKLPSTTL

SEQ ID NO: 91 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 92 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSQAMLRLLQSAFSKNSPPKQSPKKSPSAKLNIPYENFYEALEKLNKPSQLKDSEDSLYNDYVDVFYNTKPYKHRDDRLLQALVDILNEEN

SEQ ID NO: 93 - protein, 288 aa, Homo sapiens; identical to SEQ ID NO: 9

SEQ ID NO: 94 - protein, 290 aa, Homo sapiens; identical to SEQ ID NO: 11

SEQ ID NO: 95 - protein, 151 aa, Homo sapiens
MEDEGNKLCQAPPWPGQTSPVMAEYGTLLQDLTNNITLEDLEQLKSACKEDIPSEKSEEITTGSAWFSFLESHNKLDKDNLSYIEHIFEISRRPDLLTMVVDYRTRVLKISEEDELDTKLTRIPSAKKYKDIIRQPSEEEIIKLAPPPKKA

SEQ ID NO: 96 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 97 - protein, 672 aa, Homo sapiens
MADVFPGNDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSVCKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 98 - protein, 695 aa, Homo sapiens
MRERQRLPERSGSPTAGPTMAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIYQGFEKKTGVAGEDMQDNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED

SEQ ID NO: 99 - protein, 673 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKACGRNAENFDRFFTRHPPVLTPPDQEVIRNIDQSEFEGFSFVNSEFLKPEVKS

SEQ ID NO: 100 - protein, 291 aa, Homo sapiens
MERNSSLWKNLIDEHPVCTTWKQEAEGAIYHLASILFVVGFMGGSGFFGLLYVFSLLGLGFLCSAVWAWVDVCAADIFSWNFVLFVICFMQFVHIAYQVRSITFAREFQVLYSSLFQPLGISLPVFRTIALSSEVVTLEKEHCYAMQGKTSIDKLSLLVSGRIRVTVDGEFLHYIFPLQFLDSPEWDSLRPTEEGIFQVTLTAETDCRYVSWRRKKLYLLFAQHRYISRLFSVLIGSDIADKLYALNDRVYIGKRYHYDIRLPNFYQMSTPEIRRSPLTQHFQNSRRYCDK

SEQ ID NO: 101 - protein, 97 aa, Homo sapiens
MCSSLEQALAVLVTTFHKYSCQEGDKFKLSKGEMKELLHKELPSFVGEKVDEEGLKKLMGSLDENSDQQVDFQEYAVFLALITVMCNDFFQGCPDRP

SEQ ID NO: 102 - protein, 435 aa, Homo sapiens
MEGQTPGSRGLPEKPHPATAAATLSSMGAVFILMKSALGAGLLNFPWAFSKAGGVVPAFLVELVSLVFLISGLVILGYAAAVSGQATYQGVVRGLCGPAIGKLCEACFLLNLLMISVAFLRVIGDQLEKLCDSLLSGTPPAPQPWYADQRFTLPLLSVLVILPLSAPREIAFQKYTSILGTLAACYLALVITVQYYLWPQGLVRESHPSLSPASWTSVFSVFPTICFGFQCHEAAVSIYCSMRKRSLSHWALVSVLSLLACCLIYSLTGVYGFLTFGTEVSADVLMSYPGNDMVIIVARVLFAVSIVTVYPIVLFLGRSVMQDFWRRSCLGGWGPSALADPSGLWVRMPLTILWVTVTLAMALFMPDLSEIVSIIGGISSFFIFIFPGLCLICAMGVEPIGPRVKCCLEVWGVVSVLVGTFIFGQSTAAAVWEMF

SEQ ID NO: 103 - protein, 313 aa, Homo sapiens
MLLLADMDVVNQLVAGGQFRVVKEPLGFVKVLQWVFAIFAFATCGSYSGELQLSVDCANKTESDLSIEVEFEYPFRLHQVYFDAPTCRGGTTKVFLVGDYSSSAEFFVTVAVFAFLYSMGALATYIFLQNKYRENNKGPMLDFLATAVFAFMWLVSSSAWAKGLSDVKMATDPENIIKEMPVCRQTGNTCKELRDPVTSGLNTSVVFGFLNLVLWVGNLWFVFKETGWAAPFLRAPPGAPEKQPAPGDAYGDAGYGQGPGGYGPQDSYGPQGGYQPDYGQPAGSGGSGYGPQGDYGQQGYGPQGAPTSFSNQM

SEQ ID NO: 104 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 105 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 106 - protein, 729 aa, Homo sapiens
MMDASKELRSHFVAQAGVLWLFTAAIIAHCSLKLLGSSDSPTSVSRVTGITELQVLHIDFLNQDNAVSHHTWEFQTSSPVFRRGQVFHLRLVLNQPLQSYHQLKLEFSTGPNPSIAKHTLVVLDPRTPSDHYNWQATLQNESGKEVTVAVTSSPNAILGKYQLNVKTGNHILKSEENILYLLFNPWCKEDMVFMPDEDERKEYILNDTGCHYVGAARSIKCKPWNFGQFEKNVLDCCISLLTESSLKPTDRRDPVLVCRAMCAMMSFEKGQGVLIGNWTGDYEGGTAPYKWTGSAPILQQYYNTKQAVCFGQCWVFAGILTTVLRALGIPARSVTGFDSAHDTERNLTVDTYVNENGEKITSMTHDSVWNFHVWTDAWMKRPDLPKGYDGWQAVDATPQERSQGVFCCGPSPLTAIRKGDIFIVYDTRFVFSEVNGDRLIWLVKMVNGQEELHVISMETTSIGKNISTKAVGQDRRRDITYEYKYPEGSSEERQVMDHAFLLLSSEREHRRPVKENFLHMSVQSDDVLLGNSVNFTVILKRKTAALQNVNILGSFELQLYTGKKMAKLCDLNKTSQIQGQVSEVTLTLDSKTYINSLAILDDEPVIRGFIIAEIVESKEIMASEVFTSFQYPEFSIELPNTGRIGQLLVCNCIFKNTLAIPLTDVKFSLESLGISSLQTSDHGTVQPGETIQSQIKCTPIKTGPKKFIVKLSSKQVKEINAQKIVLITK

SEQ ID NO: 107 - protein, 270 aa, Homo sapiens
MARFALTVVRHGETRFNKEKIIQGQGVDEPLSETGFKQAAAAGIFLNNVKFTHAFSSDLMRTKQTMHGILERSKFCKDMTVKYDSRLRERKYGVVEGKALSELRAMAKAAREECPVFTPPGGETLDQVKMRGIDFFEFLCQLILKEADQKEQFSQGSPSNCLETSLAEIFPLGKNHSSKVNSDSGIPGLAASVLVVSHGAYMRSLFDYFLTDLKCSLPATLSRSELMSVTPNTGMSLFIINFEEGREVKPTVQCICMNLQDHLNGLTETR

SEQ ID NO: 108 - protein, 296 aa, Homo sapiens
MTKTALLKLFVAIVITFILILPEYFKTPKERTLELSCLEVCLQSNFTYSLSSLNFSFVTFLQPVRETQIIMRIFLNPSNFRNFTRTCQDITGEFKMCSSCLVCESKGNMDFISQEQTSKVLIRRGSMEVKANDFHSPCQHFNFSVAPLVDHLEEYNTTCHLKNHTGRSTIMEDEPSKEKSINYTCRIMEYPNDCIHISLHLEMDIKNITCSMKITWYILVLLVFIFLIILTIRKILEGQRRVQKWQSHRDKPTSVLLRGSDSEKLRALNVQVLSAETTQRLPLDQVQEVLPPIPEL

SEQ ID NO: 109 - protein, 905 aa, Homo sapiens
MEGESSRFEIHTPVSDKKKKKCSIHKERPQKHSHEIFRDSSLVNEQSQITRRKKRKKDFQHLISSPLKKSRICDETANATSTLKKRKKRRYSALEVDEEAGVTVVLVDKENINNTPKHFRKDVDVVCVDMSIEQKLPRKPKTDKFQVLAKSHAHKSEALHSKVREKKNKKHQRKAASWESQRARDTLPQSESHQEESWLSVGPGGEITELPASAHKNKSKKKKKKSSNREYETLAMPEGSQAGREAGTDMQESQPTVGLDDETPQLLGPTHKKKSKKKKKKKSNHQEFEALAMPEGSQVGSEVGADMQESRPAVGLHGETAGIPAPAYKNKSKKKKKKSNHQEFEAVAMPESLESAYPEGSQVGSEVGTVEGSTALKGFKESNSTKKKSKKRKLTSVKRARVSGDDFSVPSKNSESTLFDSVEGDGAMMEEGVKSRPRQKKTQACLASKHVQEAPRLEPANEEHNVETAEDSEIRYLSADSGDADDSDADLGSAVKQLQEFIPNIKDRATSTIKRMYRDDLERFKEFKAQGVAIKFGKFSVKENKQLEKNVEDFLALTGIESADKLLYTDRYPEEKSVITNLKRRYSFRLHIGRNIARPWKLIYYRAKKMFDVNNYKGRYSEGDTEKLKMYHSLLGNDWKTIGEMVARSSLSVALKFSQISSQRNRGAWSKSETRKLIKAVEEVILKKMSPQELKEVDSKLQENPESCLSIVREKLYKGISWVEVEAKVQTRNWMQCKSKWTEILTKRMTNGRRIYYGMNALRAKVSLIERLYEINVEDTNEIDWEDLASAIGDVPPSYVQTKFSRLKAVYVPFWQKKTFPEIIDYLYETTLPLLKEKLEKMMEKKGTKIQTPAAPKQVFPFRDIFYYEDDSEGEDIEKESEGQAPCMAHACNSSTLGGQGRWII

SEQ ID NO: 110 - protein, 223 aa, Homo sapiens [sequence omitted]

SEQ ID NO: 111 - protein, 523 aa, Homo sapiens
MVGQRVLLLVAFLLSGVLLSEAAKILTISTLGGSHYLLLDRVSQILQEHGHNVTMLHQSGKFLIPDIKEEEKSYQVIRWFSPEDHQKRIKKHFDSYIETALDGRKESEALVKLMEIFGTQCSYLLSRKDIMDSLKNENYDLVFVEAFDFCSFLIAEKLVKPFVAILPTTFGSLDFGLPSPLSYVPVFPSLLTDHMDFWGRVKNFLMFFSFSRSQWDMQSTFDNTIKEHFPEGSRPVLSHLLLKAELWFVNSDFAFDFARPLLPNTVYIGGLMEKPIKPVPQDLDNFIANFGDAGFVLVAFGSMLNTHQSQEVLKKMHNAFAHLPQGVIWTCQSSHWPRDVHLATNVKIVDWLPQSDLLAHPSIRLFVTHGGQNSVMEAIRHGVPMVGLPVNGDQHGNMVRVVAKNYGVSIRLNQVTADTLTLTMKQVIEDKRYKSAVVAASVILHSQPLSPAQRLVGWIDHILQTGGATHLKPYAFQQPWHEQYLIDVFVFLLGLTLGTMWLCGKLLGVVARWLRGARKVKKT

SEQ ID NO: 112 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 113 - protein, 1102 aa, Homo sapiens
MRTSLQAVALWGQKAPPHSITAIMITDDQRTIVTGSQEGQLCLWNLSHELKISAKELLFGHSASVTCLARARDFSKQPYIVSAAENGEMCVWNVTNGQCMEKATLPYRHTAICYYHCSFRMTGEGWLLCCGEYQDVLIIDAKTLAVVHSFRSSQFPDWINCMCIVHSMRIQEDSLLVVSVAGELKVWDLSSSINSIQEKQDVYEKESKFLESLNCQTIRFCTYTERLLLVVFSKCWKVYDYCDFSLLLTEVSRNGQFFAGGEVIAAHRILIWTEDGHSYIYQLLNSGLSKSIYPADGRVLKETIYPHLLCSTSVQENKEQSRPFVMGYMNERKEPFYKVLFSGEVSGRITLWHIPDVPVSKFDGSPREIPVTATWTLQDNFDKHDTMSQSIIDYFSGLKDGAGTAVVTSSEYIPSLDKLICGCEDGTIIITQALNAAKARLLEGGSLVKDSPPHKVLKGHHQSVTSLLYPHGLSSKLDQSWMLSGDLDSCVILWDIFTEEILHKFFLEAGPVTSLLMSPEKFKLRGEQIICCVCGDHSVALLHLEGKSCLLHARKHLFPVRMIKWHPVENFLIVGCADDSVYIWEIETGTLERHETGERARIILNCCDDSQLVKSVLPIASETLKHKSIEQRSSSPYQLGPLPCPGLQVESSCKVTDAKFCPRPFNVLPVKTKWSNVGFHILLFDLENLVELLLPTPLSDVDSSSSFYGGEVLRRAKSTVEKKTLTLRKSKTACGPLSAEALAKPITESLAQGDNTIKFSEENDGIKRQKKMKISKKMQPKPSRKVDASLTIDTAKLFLSCLLPWGVDKDLDYLCIKHLNILKLQGPISLGISLNEDNFSLMLPGWDLCNSGMIKDYSGVNLFSRKVLDLSDKYTATLPNQVGIPRGLENNCDSLRESDTIVYLLSRLFLVNKLVNMPLELACRVGSSFRMESIHNKMRGAGNDILNMSSFYSCLRNGKNESHVPEADLSLLKLISCWRDQSVQVTEAIQAVLLAEVQQHMKSLGKIPVNSQPVSMAENGNCEMKQMLPKLEWTEELELQCVRNTLPLQTPVSPVKHDSNSNSANFQDVEDMPDRCALEESESPGEPRHHSWIAKVCPCKVS

SEQ ID NO: 114 - protein, 504 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 115 - protein, 229 aa, Homo sapiens
MKSSDIDQDLFTDSYCKVCSAQLISESQRVAHYESRKHASKVRLYYMLHPRDGGCPAKRLRSENGSDADMVDKNKCCTLCNMSFTSAVVADSHYQGKIHAKRLKLLLGEKTPLKTTATPLSPLKPPRMDTAPVVASPYQRRDSDRYCGLCAAWFNNPLMAQQHYDGKKHKKNAARVALLEQLGTTLDMGELRGLRRNYRCTICSVSLNSIEQYHAHLKGSKHQTNLKNK

SEQ ID NO: 116 - protein, 609 aa, Homo sapiens
MEPPRGPPANGAEPSRAVGTVKVYLPNKQRTVVTVRDGMSVYDSLDKALKVRGLNQDCCVVYRLIKGRKTVTAWDTAIAPLDGEELIVEVLEDVPLTMHNFVRKTFFSLAFCDFCLKFLFHGFRCQTCGYKFHQHCSSKVPTVCVDMSTNRQQPSRFYHSVQDLSGGSRQHEAPSNRPLNELLTPQGPSPRTQHCDPEHFPFPAPANAPLQRIRSTSTPNVHMVSTTAPMDSNLIQLTGQSFSTDAAGSRGGSDGTPRGSPSPASVSSGRKSPHSKSPAEQRERKSLADDKKKVKNLGYRDSGYYWEVPPSEVQLLKRIGTGSFGTVFRGRWHGDVAVKVLKVSQPTAEQAQAFKNEMQVLRKTRHVNILLFMGFMTRPGFAIITQWCEGSSLYHHLHVADTRFDMVQLIDVARQTAQGMDYLHAKNIIHRDLKSNNIFLHEGLTVKIGDFGLATVKTRWSGAQPLEQPSGSVLWMAAEVIRMQDPNPYSFQSDVYAYGVVLYELMTGSLPYSHIGCRDQIIFMVGRGYLSPDLSKISSNCPKAMRRLLSDCLKFQREERPLFPQILATIELLQRSLPKIERSASEPSLHRTQADELPACLLSAARLVP

SEQ ID NO: 117 - protein, 186 aa, Homo sapiens
MEPPRGPPANGAEPSRAVGTVKVYLPNKQRTVVTVRDGMSVYDSLDKALKVRGLNQDCCVVYRLIKGRKTVTAWDTAIAPLDGEELIVEVLEDVPLTMHNFVRKTFFSLAFCDFCLKFLFHGFRCQTCGYKFHQHCSSKVPTVCVDMSTNRQQFYHSVQDLSGGSRQHEAPSNRPLNELLTPQGPR

SEQ ID NO: 118 - protein, 606 aa, Homo sapiens [sequence omitted]

SEQ ID NO: 119 - protein, 729 aa, Homo sapiens
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGEPYHDIRFNLMAVVPDRRIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMVRPGEPLSGEKYSPKELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 120 - protein, 752 aa, Homo sapiens
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGEPYHDIRFNLMAVVPDRRIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMVRPGEPLSGEKYSPKLPIARGALLLARPDQRCNAGFWQELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 121 - protein, 737 aa, Homo sapiens
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGEPYHDIRFNLMAVVPDRRIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMLPIARGALLLARPDQRCNAGFWQELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 122 - protein, 734 aa, Homo sapiens
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMVRPGEPLSGEKYSPKLPIARGALLLARPDQRCNAGFWQELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 123 - protein, 714 aa, Homo sapiens
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGEPYHDIRFNLMAVVPDRRIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 124 - protein, 711 aa, Homo sapiens
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMVRPGEPLSGEKYSPKELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 125 - protein, 464 aa, Homo sapiens
MLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQNAVSGFQSLLHSLAHGPFLHLQGSARVLHNGLVMEMEHPTVGKISVPGPAVRYSKFKMSEARPPPLLGQHTTHILKEVLRYDDRAIGELLSAGVVDQHETH

SEQ ID NO: 126 - protein, 390 aa, Homo sapiens
MLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPEVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQVLHNGLVMEMEHPTVGKISVPGPAVRYSKFKMSEARPPPLLGQHTTHILKEVLRYDDRAIGELLSAGVVDQHETH

SEQ ID NO: 127 - protein, 438 aa, Homo sapiens
MLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQVLHNGLVMEMEHPTVGKISVPGPAVRYSKFKMSEARPPPLLGQHTTHILKEVLRYDDRAIGELLSAGVVDQHETH

SEQ ID NO: 128 - protein, 427 aa, Homo sapiens
MLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQNAVSGFQSLLHSLAHGPFLHLQGSARVLHNGLVMEMEHPTVGKISVPGPAVRYSKFKMSEARPPPLLGQHTTHILKEVLRYDDRAIGELLSAGVVDQHETH

SEQ ID NO: 129 - protein, 471 aa, Homo sapiens
MPSETHAMLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQVQSVEWLHKGDILMKKTLYNKTITITVLHNGLVMEMEHPTVGKISVPGPAVRYSKFKMSEARPPPLLGQHTTHILKEVLRYDDRAIGELLSAGVVDQHETH

SEQ ID NO: 130 - protein, 497 aa, Homo sapiens
MPSETHAMLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQNAVSGFQSLLHSLAHGPFLHLQGSARVQSVEWLHKGDILMKKTLYNKTITITVLHNGLVMEMEHPTVGKISVPGPAVRYSKFKMSEARPPPLLGQHTTHILKEVLRYDDRAIGELLSAGVVDQHETH

SEQ ID NO: 131 - protein, 471 aa, Homo sapiens
MYFYRRMRPYRWNNDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQNAVSGFQSLLHSLAHGPFLHLQGSARVQSVEWLHKGDILMKKTLYNKTITITVLHNGLVMEMEHPTVGKISVPGPAVRYSKFKMSEARPPPLLGQHTTHILKEVLRYDDRAIGELLSAGVVDQHETH

SEQ ID NO: 132 - protein, 449 aa, Homo sapiens
MPSETHAMLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPEVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQNAVSGFQSLLHSLAHGPFLHLQGSARVQSVEWLHKGDILMKKTLYNKTITITVLHNGLVMEMEHPTVGKISVPGPAVRYSKFKMSEARPPPLLGQHTTHILKEVLRYDDRAIGELLSAGVVDQHETH

SEQ ID NO: 133 - protein, 426 aa, Homo sapiens
MPSETHAMLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQVSSLVLPLMSCRVSWRLVHGNGHFCSDVGTHHWRVWPTLHVRCASTAASSTGNCFG

SEQ ID NO: 134 - protein, 423 aa, Homo sapiens
MPSETHAMLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPEVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQNAVSGFQSLLHSLAHGPFLHLQGSARVLHNGLVMEMEHPTVGKISVPGPAVRYSKFKMSEARPPPLLGQHTTHILKEVLRYDDRAIGELLSAGVVDQHETH

SEQ ID NO: 135 - protein, 296 aa, Homo sapiens
MPSETHAMLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQMRKRVCYFPEETQPVSG

SEQ ID NO: 136 - protein, 456 aa, Homo sapiens
MNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQNAVSGFQSLLHSLAHGPFLHLQGSARVQSVEWLHKGDILMKKTLYNKTITITVLHNGLVMEMEHPTVGKISVPGPAVRYSKFKMSEARPPPLLGQHTTHILKEVLRYDDRAIGELLSAGVVDQHETH

SEQ ID NO: 137 - protein, 435 aa, Homo sapiens
MPSETHAMLATLARVAALRRTCLFSGRGGGRGLWTGRPQSDMNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQSLNRHEWAPTMYLPLCRAVESSQKWRRKENTYNLEKLRPEENLKNTLIKEDINCEGNTEMGITTL

SEQ ID NO: 138 - protein, 248 aa, Homo sapiens
MFLAKALLEGADRGLGEALGGLFGGGGQRREGGGRNIGGIVGGIVNFISEAAAAQYTPEPPPTQQHFTSVEASESEEVRRFRQQFTQLAGPDMEVGATDLMNILNKVLSKHKDLKTDGFSLDTCRSIVSVMDSDTTGKLGFEEFKYLWNNIKKWQCVYKQYDRDHSGSLGSSQLRGALQAAGFQLNEQLYQMIVRRYANEDGDMDFNNFISCLVRLDAMFRAFKSLDRDRDGLIQVSIKEWLQLTMYS

SEQ ID NO: 139 - protein, 1156 aa, Homo sapiens
MASLLPLLCLCVVAAHLAGARDATPTEEPMATALGLERRSVYTGQPSPALEDWEEASEWTSWFNVDHPGGDGDFESLAAIRFYYGPARVCPRPLALEARTTDWALPSAVGERVHLNPTRGFWCLNREQPRGRRCSNYHVRFRCPLEASWGAWGPWGPCSGSCGPGRRLRRRHCPSPAGDACPGRPLEAQKCVRPRCPGCSLDTCECPDHILLGSVVTPSGQPLLGARVSLRDQPGTVATSDAHGTFRVPGVCADSRANIRAQMDGFSAGEAQAQANGSISVVTIILDKLEKPYLVKHPESRVREAGQNVTFCCKASGTPMPKKYSWFHNGTLLDRRAHGYGAHLELRGLRPDQAGIYHCKAWNEAGAVRSGTARLTVLAPGQPACDPRPREYLIKLPEDCGQPGSGPAYLDVGLCPDTRCPSLAGSSPRCGDASSRCCSVRRLERREIHCPGYVLPVKVVAECGCQKCLPPRGLVRGRVVAADSGEPLRFARILLGQEPIGFTAYQGDFTIEVPPSTQRLVVTFVDPSGEFMDAVRVLPFDPRGAGVYHEVKAMRKKAPVILHTSQSNTIPLGELEDEAPLGELVLPSGAFRRADGKPYSGPVEARVTFVDPRDLTSAASAPSDLRFVDSDGELAPLRTYGMFSVDLRAPGSAEQLQVGPVAVRVAASQIHMPGHVEALKLWSLNPETGLWEEESGFRREGSSGPRVRREERVFLVGNVEIRERRLFNLDVPERRRCFVKVRAYANDKFTPSEQVEGVVVTLVNLEPAPGFSANPRAWGRFDSAVTGPNGACLPAFCDADRPDAYTALVTATLGGEELEPAPSLPRPLPATVGVTQPYLDRLGYRRTDHDDPAFKRNGFRINLAKPRPGDPAEANGPVYPWRSLRECQGAPVTASHFRFARVEADKYEYNVVPFREGTPASWTGDLLAWWPNPQEFRACFLKVKIQGPQEYMVRSHNAGGSHPRTRGQLYGLRDARSVRDPERPGTSAACVEFKCSGMLFDQRQVDRTLVTIMPQGSCRRVAVNGLLRDYLTRHPPPVPAEDPAAFSMLAPLDPLGHNYGVYTVTDQSPRLAKEIAIGRCFDGSSDGFSREMKADAGTAVTFQCREPPAGRPSLFQRLLESPATALGDIRREMSEAAQAQARASGPLRTRRGRVRQ

SEQ ID NO: 140 - protein, 211 aa, Homo sapiens
MANSGLQLLGFSMALLGWVGLVACTAIPQWQMSSYAGDNIITAQAMYKGLWMDCVTQSTGMMSCKMYDSVLALSAALQATRALMVVSLVLGFLAMFVATMGMKCTRCGGDDKVKKARIAMGGGIIFIVAGLAALVACSWYGHQIVTDFYNPLIPTNIKYEFGPAIFIGWAGSALVILGGALLSCSCPGNESKAGYRVPRSYPKSNSSKEYV

SEQ ID NO: 141 - protein, 145 aa, Homo sapiens
MANSGLQLLGFSMALLGWVGLVACTAIPQWQMSSYAGDNIITAQAMYKGLWMDCVTQSTGMMSCKMYDSVLALSAALQATRALMVVSLVLGFLAMFVATMGMKCTRCGGDDKVKKARIAMGGGIIFIVAGMSLALPSLLAGQGLP

SEQ ID NO: 142 - protein, 211 aa, Homo sapiens; identical to SEQ ID NO: 140

SEQ ID NO: 143 - protein, 1390 aa, Homo sapiens; identical to SEQ ID NO: 5

SEQ ID NO: 144 - protein, 1400 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTR

SEQ ID NO: 145 - protein, 934 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 146 - protein, 960 aa, Homo sapiens
MGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 147 - protein, 956 aa, Homo sapiens
MGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIKDFSHPNVLSLLGICLRSEGSPLVVLPYMKHGDLRNFIRNETHNPTVKDLIGFGLQVAKGMKYLASKKFHRDLAARNCMLDEKFTVKVADFGLARDMYDKEYYSVHNKTGAKLPVKWMALESLQTQKFTTKSDVWSFGLLWELMTRGAPPYPDVNTFDITVYLLQGRRLLQPEYCPDPLYEVMLKCWHPKAEMRPSFSELVSRISAISTFIGEHYVHVNATYVNVKCVAPYPSLLSSEDNADDEVDTRPASFWETS

SEQ ID NO: 148 - protein, 1409 aa, Homo sapiens
MKSKSKSLAECFPYDKPLIMKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 149 - protein, 638 aa, Homo sapiens
MPLPLLPMDLKGEPGPPGKPGPRGPPGPPGFPGKPGMGKPGLHGQPGPAGPPGFSRMGKAGPPGLPGKVGPPGQPGLRGEPGIRGDQGLRGPPGPPGLPGPSGITIPGKPGAQGVPGPPGFQGEPGPQGEPGPPGDRGLKGDNGVGQPGLPGAPGQGGAPGPPGLPGPAGLGKPGLDGLPGAPGDKGESGPPGVPGPRGEPGAVGPKGPPGVDGVGVPGAAGLPGPQGPSGAKGEPGTRGPPGLIGPTGYGMPGLPGPKGDRGPAGVPGLLGDRGEPGEDGEPGEQGPQGLGGPPGLPGSAGLPGRRGPPGPKGEAGPGGPPGVPGIRGDQGPSGLAGKPGVPGERGLPGAHGPPGPTGPKGEPGFTGRPGGPGVAGALGQKGDLGLPGQPGLRGPSGIPGLQGPAGPIGPQGLPGLKGEPGLPGPPGEGRAGEPGTAGPTGPPGVPGSPGITGPPGPPGPPGPPGAPGAFDETGIAGLHLPNGGVEGAVLGKGGKPQFGLGELSAHATPAFTAVLTSPFPASGMPVKFDRTLYNGHSGYNPATGIFTCPVGGVYYFAYHVHVKGTNVWVALYKNNVPATYTYDEYKKGYLDQASGGAVLQLRPNDQVWVQMPSDQANGLYSTEYIHSSFSGFLLCPT

SEQ ID NO: 150 - protein, 703 aa, Homo sapiens
MLGTLTPLSSLLLLLLVLVLGCGPRASSGGGAGGAAGYAPVKYIQPMQKGPVGPPFREGKGQYLEMPLPLLPMDLKGEPGPPGKPGPRGPPGPPGFPGKPGMGKPGLHGQPGPAGPPGFSRMGKAGPPGLPGKVGPPGQPGLRGEPGIRGDQGLRGPPGPPGLPGPSGITIPGKPGAQGVPGPPGFQGEPGPQGEPGPPGDRGLKGDNGVGQPGLPGAPGQGGAPGPPGLPGPAGLGKPGLDGLPGAPGDKGESGPPGVPGPRGEPGAVGPKGPPGVDGVGVPGAAGLPGPQGPSGAKGEPGTRGPPGLIGPTGYGMPGLPGPKGDRGPAGVPGLLGDRGEPGEDGEPGEQGPQGLGGPPGLPGSAGLPGRRGPPGPKGEAGPGGPPGVPGIRGDQGPSGLAGKPGVPGERGLPGAHGPPGPTGPKGEPGFTGRPGGPGVAGALGQKGDLGLPGQPGLRGPSGIPGLQGPAGPIGPQGLPGLKGEPGLPGPPGEGRAGEPGTAGPTGPPGVPGSPGITGPPGPPGPPGPPGAPGAFDETGIAGLHLPNGGVEGAVLGKGGKPQFGLGELSAHATPAFTAVLTSPFPASGMPVKFDRTLYNGHSGYNPATGIFTCPVGGVYYFAYHVHVKGTNVWVALYKNNVPATYTYDEYKKGYLDQASGGAVLQLRPNDQVWVQMPSDQANGLYSTEYIHSSFSGFLLCPT

SEQ ID NO: 151 - protein, 780 aa, Homo sapiens
MCGSRLAPVSPPGSAYRGHVCRQPSGDSSLGATSPSCPFPAGPRLAVLLSSRVRPHRPCLCWNLPRPLRPESTSTDAMLGTLTPLSSLLLLLLVLVLGCGPRASSGGGAGGAAGYAPVKYIQPMQKGPVGPPFREGKGQYLEMPLPLLPMDLKGEPGPPGKPGPRGPPGPPGFPGKPGMGKPGLHGQPGPAGPPGFSRMGKAGPPGLPGKVGPPGQPGLRGEPGIRGDQGLRGPPGPPGLPGPSGITIPGKPGAQGVPGPPGFQGEPGPQGEPGPPGDRGLKGDNGVGQPGLPGAPGQGGAPGPPGLPGPAGLGKPGLDGLPGAPGDKGESGPPGVPGPRGEPGAVGPKGPPGVDGVGVPGAAGLPGPQGPSGAKGEPGTRGPPGLIGPTGYGMPGLPGPKGDRGPAGVPGLLGDRGEPGEDGEPGEQGPQGLGGPPGLPGSAGLPGRRGPPGPKGEAGPGGPPGVPGIRGDQGPSGLAGKPGVPGERGLPGAHGPPGPTGPKGEPGFTGRPGGPGVAGALGQKGDLGLPGQPGLRGPSGIPGLQGPAGPIGPQGLPGLKGEPGLPGPPGEGRAGEPGTAGPTGPPGVPGSPGITGPPGPPGPPGPPGAPGAFDETGIAGLHLPNGGVEGAVLGKGGKPQFGLGELSAHATPAFTAVLTSPFPASGMPVKFDRTLYNGHSGYNPATGIFTCPVGGVYYFAYHVHVKGTNVWVALYKNNVPATYTYDEYKKGYLDQASGGAVLQLRPNDQVWVQMPSDQANGLYSTEYIHSSFSGFLLCPT

SEQ ID NO: 152 - protein, 298 aa, Homo sapiens
MACIENVLGGHAPSPLVVSVDKNGNQELHHDMPLQCLSSKPEDDAEPWGQPQVPLRPSVNVLTDLDSKQLEWPSERTGSCIPLHSLRAHRHPYGPPPAVAEESLATAEVNSSDALAGWRQEGQDAINVSWEVSGGPPALIVGGTKVNNGGTERGSNNARLHVALPQGKGFFPPRGPQVRGPSHIPTLRSGIVMEVPPGNTRIACRGKLAHVSFPLRGPCHPMHNWPRPIPLSSSTPGLPSCSTVHCFIPPRPPIFNPFLTMPLPFAPPPIFGPPLPSYFAHFHSGGMPAPASPNREHS

SEQ ID NO: 153 - protein, 410 aa, Homo sapiens
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 154 - protein, 365 aa, Homo sapiens
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKLLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 155 - protein, 361 aa, Homo sapiens
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 156 - protein, 395 aa, Homo sapiens
MKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 157 - protein, 407 aa, Homo sapiens
MPRERRERDAKERDTMKEDGGAEFSARSRKRKANVTVDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 158 - protein, 497 aa, Homo sapiens
MGLPALLASALCTFVLPLLLFLAAIKLWDLYCVSGRDRSCALPLPPGTMGFPFFGETLQMVLQRRKFLQMKRRKYGFIYKTHLFGRPTVRVMGADNVRRILLGEHRLVSVHWPASVRTILGSGCLSNLHDSSHKQRKKVIMRAFSREALECYVPVITEEVGSSLEQWLSCGERGLLVYPEVKRLMFRIAMRILLGCEPQLAGDGDSEQQLVEAFEEMTRNLFSLPIDVPFSGLYRGMKARNLIHARIEQNIRAKICGLRASEAGQGCKDALQLLIEHSWERGERLDMQALKQSSTELLFGGHETTASAATSLITYLGLYPHVLQKVREELKSKGLLCKSNQDNKLDMEILEQLKYIGCVIKETLRLNPPVPGGFRVALKTFELNGYQIPKGWNVIYSICDTHDVAEIFTNKEEFNPDRFMLPHPEDASRFSFIPFGGGLRSCVGKEFAKILLKIFTVELARHCDWQLLNGPPTMKTSPTVYPVDNLPARFTHFHGEI

SEQ ID NO: 159 - protein, 428 aa, Homo sapiens
MKRRKYGFIYKTHLFGRPTVRVMGADNVRRILLGEHRLVSVHWPASVRTILGSGCLSNLHDSSHKQRKKVIMRAFSREALECYVPVITEEVGSSLEQWLSCGERGLLVYPEVKRLMFRIAMRILLGCEPQLAGDGDSEQQLVEAFEEMTRNLFSLPIDVPFSGLYRGMKARNLIHARIEQNIRAKICGLRASEAGQGCKDALQLLIEHSWERGERLDMQALKQSSTELLFGGHETTASAATSLITYLGLYPHVLQKVREELKSKGLLCKSNQDNKLDMEILEQLKYIGCVIKETLRLNPPVPGGFRVALKTFELNGYQIPKGWNVIYSICDTHDVAEIFTNKEEFNPDRFMLPHPEDASRFSFIPFGGGLRSCVGKEFAKILLKIFTVELARHCDWQLLNGPPTMKTSPTVYPVDNLPARFTHFHGEI

SEQ ID NO: 160 - protein, 922 aa, Homo sapiens
MSQFKRQRINPLPGGRNFSGTASTSLLGPPPGLLTPPVATELSQNARHLQGGEKQRVFTGIVTSLHDYFGVVDEEVFFQLSVVKGRLPQLGEKVLVKAAYNPGQAVPWNAVKVQTLSNQPLLKSPAPPLLHVAALGQKQGILGAQPQLIFQPHRIPPLFPQKPLSLFQTSHTLHLSHLNRFPARGPHGRLDQGRSDDYDSKKRKQRAGGEPWGAKKPRHDLPPYRVHLTPYTVDSPICDFLELQRRYRSLLVPSDFLSVHLSWLSAFPLSQPFSLHHPSRIQVSSEKEAAPDAGAEPITADSDPAYSSKVLLLSSPGLEELYRCCMLFVDDMAEPRETPEHPLKQIKFLLGRKEEEAVLVGGEWSPSLDGLDPQADPQVLVRTAIRCAQAQTGIDLSGCTKWWRFAEFQYLQPGPPRRLQTVVVYLPDVWTIMPTLEEWEALCQQKAAEAAPPTQEAQGETEPTEQAPDALEQAADTSRRNAETPEATTQQETDTDLPEAPPPPLEPAVIARPGCVNLSLHGIVEDRRPKERISFEVMVLAELFLEMLQRDFGYRVYKMLLSLPEKVVSPPEPEKEEAAKEEATKEEEAIKEEVVKEPKDEAQNEGPATESEAPLKEDGLLPKPLSSGGEEEEKPRGEASEDLCEMALDPELLLLRDDGEEEFGAKLEDSEVRSVASNQSEMEFSSLQDMPKELDPSAVLPLDCLLAFVFFDANWCGYLHRRDLERILLTLGIRLSAEQAKQLVSRVVTQNICQYRSLQYSRQEGLDGGLPEEVLFGNLDLLPPPGKSTKPGAAPTEHKALVSHNGSLINVGSLLQRAEQQDSGRLYLENKIHTLELKLEESHNRFSATEVTNKTLAAEMQELRVRLAEAEETARTAERQKSQLQRLLQELRRRLTPLQLEIQRVVEKADSWVEKEEPAPSN

SEQ ID NO: 161 - protein, 922 aa, Homo sapiens; identical to SEQ ID NO: 160

SEQ ID NO: 162 - protein, 923 aa, Homo sapiens
MSQFKRQRINPLPGGRNFSGTASTSLLGPPPGLLTPPVATELSQNARHLQGGEKQRVFTGIVTSLHDYFGVVDEEVFFQLSVVKGRLPQLGEKVLVKAAYNPGQAVPWNAVKVQTLSNQPLLKSPAPPLLHVAALGQKQGILGAQPQLIFQPHRIPPLFPQKPLSLFQTSHTLHLSHLNRFPARGPHGRLDQGRSDDYDSKKRKQRAGGEPWGAKKPRHDLPPYRVHLTPYTVDSPICDFLELQRRYRSLLVPSDFLSVHLSWLSAFPLSQPFSLHHPSRIQVSSEKEAAPDAGAEPITADSDPAYSSKVLLLSSPGLEELYRCCMLFVDDMAEPRETPEHPLKQIKFLLGRKEEEAVLVGGEWSPSLDGLDPQADPQVLVRTAIRCAQAQTGIDLSGCTKWWRFAEFQYLQPGPPRRLQTVVVYLPDVWTIMPTLEEWEALCQQKAAEAAPPTQEAQGETEPTEQAPDALEQAADTSRRNAETPEATTQQETDTDLPEAPPPPLEPAVIARPGCVNLSLHGIVEDRRPKERISFEVMVLAELFLEMLQRDFGYRVYKMLLSLPEKVVSPPEPEKEEAAKEEATKEEEAIKEEVVKEPKDEAQNEGPATESEAPLKEDGLLPKPLSSGGEEEEKPRGEASEDLCEMALDPELLLLRDDGEEEFAGAKLEDSEVRSVASNQSEMEFSSLQDMPKELDPSAVLPLDCLLAFVFFDANWCGYLHRRDLERILLTLGIRLSAEQAKQLVSRVVTQNICQYRSLQYSRQEGLDGGLPEEVLFGNLDLLPPPGKSTKPGAAPTEHKALVSHNGSLINVGSLLQRAEQQDSGRLYLENKIHTLELKLEESHNRFSATEVTNKTLAAEMQELRVRLAEAEETARTAERQKSQLQRLLQELRRRLTPLQLEIQRVVEKADSWVEKEEPAPSN

SEQ ID NO: 163 - protein, 923 aa, Homo sapiens; identical to SEQ ID NO: 162

SEQ ID NO: 164 - protein, 883 aa, Homo sapiens
MSQFKRQRINPLPGGRNFSGTASTSLLGPPPGLLTPPVATELSQNARHLQGGEKQRVFTGIVTSLHDYFGVVDEEVFFQLSVVKGRLPQLGEKVLVKAAYNPGQAVPWNAVKVQTLSNQPLLKSPAPPLLHVAALGQKQGILGAQPQLIFQPHRIPPLFPQKPLSLFQTSHTLHLSHLNRFPARGPHGRLDQGRSPICDFLELQRRYRSLLVPSDFLSVHLSWLSAFPLSQPFSLHHPSRIQVSSEKEAAPDAGAEPITADSDPAYSSKVLLLSSPGLEELYRCCMLFVDDMAEPRETPEHPLKQIKFLLGRKEEEAVLVGGEWSPSLDGLDPQADPQVLVRTAIRCAQAQTGIDLSGCTKWWRFAEFQYLQPGPPRRLQTVVVYLPDVWTIMPTLEEWEALCQQKAAEAAPPTQEAQGETEPTEQAPDALEQAADTSRRNAETPEATTQQETDTDLPEAPPPPLEPAVIARPGCVNLSLHGIVEDRRPKERISFEVMVLAELFLEMLQRDFGYRVYKMLLSLPEKVVSPPEPEKEEAAKEEATKEEEAIKEEVVKEPKDEAQNEGPATESEAPLKEDGLLPKPLSSGGEEEEKPRGEASEDLCEMALDPELLLLRDDGEEEFAGAKLEDSEVRSVASNQSEMEFSSLQDMPKELDPSAVLPLDCLLAFVFFDANWCGYLHRRDLERILLTLGIRLSAEQAKQLVSRVVTQNICQYRSLQYSRQEGLDGGLPEEVLFGNLDLLPPPGKSTKPGAAPTEHKALVSHNGSLINVGSLLQRAEQQDSGRLYLENKIHTLELKLEESHNRFSATEVTNKTLAAEMQELRVRLAEAEETARTAERQKSQLQRLLQELRRRLTPLQLEIQRVVEKADSWVEKEEPAPSN

SEQ ID NO: 165 - protein, 249 aa, Homo sapiens
MGLPQPGLWLKRLWVLLEVAVHVVVGKVLLILFPDRVKRNILAMGEKTGMTRNPHFSHDNWIPTFFSTQYFWFVLKVRWQRLEDTTELGGLAPNCPVVRLSGQRCNIWEFMQGNRPLVLNFGSCTUPSFMFKFDQFKRLIEDFSSIADFLVIYIEEAHASDGWAFKNNMDIRNHQNLQDRLQAAHLLLARSPQCPVVVDTMQNQSSQLYAALPERLYIIQEGRILYKGKSGPWNYNPEEVRAVLEKLHS

SEQ ID NO: 166 - protein, 201 aa, Homo sapiens
MGLPQPGLWLKRLWVLLEVAVHVVVGKVLLILFPDRVKRNILAMGEKTGMTRNPHFSHDNWIPTFFSTQYFWFVLKVRWQRLEDTTELGGLAPNCPVVRLSGQRCNIWEFMQDGWAFKNNMDIRNHQNLQDRLQAAHLLLARSPQCPVVVDTMQNQSSQLYAALPERLYIIQEGRILYKGKSGPWNYNPEEVRAVLEKLHS

SEQ ID NO: 167 - protein, 161 aa, Homo sapiens
MGLPQPGLWLKRLWVLLEVAVHVVVGKVLLILFPDRVKRNILAMGEKTGMTRNPHFSHDNWIPTFFSTQYFWFVLKVRWQRLEDTTELGGLAPNCPVVRLSGQRCNIWEFMQGNRPLVLNFGSCTUPSFMFKFDQFKRLIEDFSSIADFLVIYIEEAHASG

SEQ ID NO: 168 - protein, 113 aa, Homo sapiens
MGLPQPGLWLKRLWVLLEVAVHVVVGKVLLILFPDRVKRNILAMGEKTGMTRNPHFSHDNWIPTFFSTQYFWFVLKVRWQRLEDTTELGGLAPNCPVVRLSGQRCNIWEFMQG

SEQ ID NO: 169 - protein, 185 aa, Homo sapiens
MGLPQPGLWLKRLWVLLEVAVHVVVGKVLLILFPDRVKRNILAMGEKTGNRPLVLNFGSCTUPSFMFKFDQFKRLIEDFSSIADFLVIYIEEAHASDGWAFKNNMDIRNHQNLQDRLQAAHLLLARSPQCPVVVDTMQNQSSQLYAALPERLYIIQEGRILYKGKSGPWNYNPEEVRAVLEKLHS

SEQ ID NO: 170 - protein, 1091 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQGQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 171 - protein, 1136 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 172 - protein, 1165 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQGQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 173 - protein, 1157 aa, Homo sapiens
MFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 174 - protein, 943 aa, Homo sapiens
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 175 - protein, 1210 aa, Homo sapiens [sequence omitted]

SEQ ID NO: 176 - protein, 628 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGS

SEQ ID NO: 177 - protein, 405 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGLS

SEQ ID NO: 178 - protein, 705 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 179 - protein, 875 aa, Homo sapiens [sequence omitted]

SEQ ID NO: 180 - protein, 621 aa, Homo sapiens
MWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 181 - protein, 798 aa, Homo sapiens
MPVFHRAQLEKCCSLARIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 182 - protein, 2446 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 183 - protein, 2267 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 184 - protein, 2265 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 185 - protein, 2240 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 186 - protein, 2387 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 187 - protein, 2386 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 188 - protein, 2362 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 189 - protein, 2361 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 190 - protein, 2356 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 191 - protein, 2331 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 192 - protein, 2271 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 193 - protein, 2266 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 194 - protein, 2355 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 195 - protein, 657 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPVSIPPRNLGY

SEQ ID NO: 196 - protein, 2176 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 197 - protein, 2296 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 198 - protein, 2330 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 199 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 200 - protein, 1894 aa, Homo sapiens
MAPEAGATLRAPRRLSWAALLLLAALLPVASSAAASVDHPLKPRHVKLLSTKMGLKVTWDPPKDATSRPVEHYNIAYGKSLKSLKYIKVNAETYSFLIEDVEPGVVYFVLLTAENHSGVSRPVYRAESPPGGEWIEIDGFPIKGPGPFNETVTEKEVPNKPLRVRVRSSDDRLSVAWKAPRLSGAKSPRRSRGFLLGYGESGRKMNYVPLTRDERTHEIKKLASESVYVVSLQSMNSQGRSQPVYRAALTKRKISEEDELDVPDDISVRVMSSQSVLVSWVDPVLEKQKKVVASRQYTVRYREKGELARWDYKQIANRRVLIENLIPDTVYEFAVRISQGERDGKWSTSVFQRTPESAPTTAPENLNVWPVNGKPTVVAASWDALPETEGKVKEYILSYAPALKPFGAKSLTYPGDTTSALVDGLQPGERYLFKIRATNRRGLGPHSKAFIVAMPTTSKADVEQNTEDNGKPEKPEPSSPSPRAPASSQHPSVPASPQGRNAKDLLLDLKNKILANGGAPRKPQLRAKKAEELDLQSTEITGEEELGSREDSPMSPSDTQDQKRTLRPPSRHGHSVVAPGRTAVRARMPALPRREGVDKPGFSLATQPRPGAPPSASASPAHHASTQGTSHRPSLPASLNDNDLVDSDEDERAVGSLHPKGAFAQPRPALSPSRQSPSSVLRDRSSVHPGAKPASPARRTPHSGAAEEDSSASAPPSRLSPPHGGSSRLLPTQPHLSSPLSKGGKDGEDAPATNSNAPSRSTMSSSVSSHLSSRTQVSEGAEASDGESHGDGDREDGGRQAEATAQTLRARPASGHFHLLRHKPFAANGRSPSRFSIGRGPRLQPSSSPQSTVPSRAHPRVPSHSDSHPKLSSGIHGDEEDEKPLPATVVNDHVPSSSRQPISRGWEDLRRSPQRGASLHRKEPIPENPKSTGADTHPQGKYSSLASKAQDVQQSTDADTEGHSPKAQPGSTDRHASPARPPAARSQQHPSVPRRMTPGRAPQQQPPPPVATSQHHPGPQSRDAGRSPSQPRLSLTQAGRPRPTSQGRSHSSSDPYTASSRGMLPTALQNQDEDAQGSYDDDSTEVEAQDVRAPAHAARAKEAAASLPKHQQVESPTGAGAGGDHRSQRGHAASPARPSRPGGPQSRARVPSRAAPGKSEPPSKRPLSSKSQQSVSAEDDEEEDAGFFKGGKEDLLSSSVPKWPSSSTPRGGKDADGSLAKEEREPAIALAPRGGSLAPVKRPLPPPPGSSPRASHVPSRLPPRSAATVSPVAGTHPWPQYTTRAPPGHFSTTPMLSLRQRMMHARFRNPLSRQPARPSYRQGYNGRPNVEGKVLPGSNGKPNGQRIINGPQGTKWVVDLDRGLVLNAEGRYLQDSHGNPLRIKLGGDGRTIVDLEGTPVVSPDGLPLFGQGRHGTPLANAQDKPILSLGGKPLVGLEVIKKTTHPPTTTMQPTTTTTPLPTTTTPRPTTATTRRTTTTRRTTTRRPTTTVRTTTRTTTTTTPTPTTPIPTCPPGTLERHDDDGNLIMSSNGIPECYAEEDEFSGLETDTAVPTEEAYVIYDEDYEFETSRPPTTTEPSTTATTPRVIPEEGAISSFPEEEFDLAGRKRFVAPYVTYLNKDPSAPCSLTDALDHFQVDSLDEIIPNDLKKSDLPPQHAPRNITVVAVEGCHSFVIVDWDKATPGDVVTGYLVYSASYEDFIRNKWSTQASSVTHLPIENLKPNTRYYFKVQAQNPHGYGPISPSVSFVTESDNPLLVVRPPGGEPIWIPFAFKHDPSYTDCHGRQYVKRTWYRKFVGVVLCNSLRYKIYLSDNLKDTFYSIGDSWGRGEDHCQFVDSHLDGRTGPQSYVEALPTIQGYYRQYRQEPVRFGNIGFGTPYYYVGWYECGVSIPGKW

SEQ ID NO: 201 - protein, 1871 aa, Homo sapiens
MAPEAGATLRAPRRLSWAALLLLAALLPVASSAAASVDHPLKPRHVKLLSTKMGLKVTWDPPKDATSRPVEHYNIAYGKSLKSLKYIKVNAETYSFLIEDVEPGVVYFVLLTAENHSGVSRPVYRAESPPEKEVPNKPLRVRVRSSDDRLSVAWKAPRLSGAKSPRRSRGFLLGYGESGRKMNYVPLTRDERTHEIKKLASESVYVVSLQSMNSQGRSQPVYRAALTKRKISEEDELDVPDDISVRVMSSQSVLVSWVDPVLEKQKKVVASRQYTVRYREKGELARWDYKQIANRRVLIENLIPDTVYEFAVRISQGERDGKWSTSVFQRTPESAPTTAPENLNVWPVNGKPTVVAASWDALPETEGKVKEYILSYAPALKPFGAKSLTYPGDTTSALVDGLQPGERYLFKIRATNRRGLGPHSKAFIVAMPTTSKADVEQNTEDNGKPEKPEPSSPSPRAPASSQHPSVPASPQGRNAKDLLLDLKNKILANGGAPRKPQLRAKKAEELDLQSTEITGEEELGSREDSPMSPSDTQDQKRTLRPPSRHGHSVVAPGRTAVRARMPALPRREGVDKPGFSLATQPRPGAPPSASASPAHHASTQGTSHRPSLPASLNDNDLVDSDEDERAVGSLHPKGAFAQPRPALSPSRQSPSSVLRDRSSVHPGAKPASPARRTPHSGAAEEDSSASAPPSRLSPPHGGSSRLLPTQPHLSSPLSKGGKDGEDAPATNSNAPSRSTMSSSVSSHLSSRTQVSEGAEASDGESHGDGDREDGGRQAEATAQTLRARPASGHFHLLRHKPFAANGRSPSRFSIGRGPRLQPSSSPQSTVPSRAHPRVPSHSDSHPKLSSGIHGDEEDEKPLPATVVNDHVPSSSRQPISRGWEDLRRSPQRGASLHRKEPIPENPKSTGADTHPQGKYSSLASKAQDVQQSTDADTEGHSPKAQPGSTDRHASPARPPAARSQQHPSVPRRMTPGRAPQQQPPPPVATSQHHPGPQSRDAGRSPSQPRLSLTQAGRPRPTSQGRSHSSSDPYTASSRGMLPTALQNQDEDAQGSYDDDSTEVEAQDVRAPAHAARAKEAAASLPKHQQVESPTGAGAGGDHRSQRGHAASPARPSRPGGPQSRARVPSRAAPGKSEPPSKRPLSSKSQQSVSAEDDEEEDAGFFKGGKEDLLSSSVPKWPSSSTPRGGKDADGSLAKEEREPAIALAPRGGSLAPVKRPLPPPPGSSPRASHVPSRLPPRSAATVSPVAGTHPWPQYTTRAPPGHFSTTPMLSLRQRMMHARFRNPLSRQPARPSYRQGYNGRPNVEGKVLPGSNGKPNGQRIINGPQGTKWVVDLDRGLVLNAEGRYLQDSHGNPLRIKLGGDGRTIVDLEGTPVVSPDGLPLFGQGRHGTPLANAQDKPILSLGGKPLVGLEVIKKTTHPPTTTMQPTTTTTPLPTTTTPRPTTATTRRTTTTRRTTTRRPTTTVRTTTRTTTTTTPTPTTPIPTCPPGTLERHDDDGNLIMSSNGIPECYAEEDEFSGLETDTAVPTEEAYVIYDEDYEFETSRPPTTTEPSTTATTPRVIPEEGAISSFPEEEFDLAGRKRFVAPYVTYLNKDPSAPCSLTDALDHFQVDSLDEIIPNDLKKSDLPPQHAPRNITVVAVEGCHSFVIVDWDKATPGDVVTGYLVYSASYEDFIRNKWSTQASSVTHLPIENLKPNTRYYFKVQAQNPHGYGPISPSVSFVTESDNPLLVVRPPGGEPIWIPFAFKHDPSYTDCHGRQYVKRTWYRKFVGVVLCNSLRYKIYLSDNLKDTFYSIGDSWGRGEDHCQFVDSHLDGRTGPQSYVEALPTIQGYYRQYRQEPVRFGNIGFGTPYYYVGWYECGVSIPGKW

SEQ ID NO: 202 - protein, 1777 aa, Homo sapiens
MAPEAGATLRAPRRLSWAALLLLAALLPVASSAAASEKEVPNKPLRVRVRSSDDRLSVAWKAPRLSGAKSPRRSRGFLLGYGESGRKMNYVPLTRDERTHEIKKLASESVYVVSLQSMNSQGRSQPVYRAALTKRKISEEDELDVPDDISVRVMSSQSVLVSWVDPVLEKQKKVVASRQYTVRYREKGELARWDYKQIANRRVLIENLIPDTVYEFAVRISQGERDGKWSTSVFQRTPESAPTTAPENLNVWPVNGKPTVVAASWDALPETEGKVKEYILSYAPALKPFGAKSLTYPGDTTSALVDGLQPGERYLFKIRATNRRGLGPHSKAFIVAMPTTSKADVEQNTEDNGKPEKPEPSSPSPRAPASSQHPSVPASPQGRNAKDLLLDLKNKILANGGAPRKPQLRAKKAEELDLQSTEITGEEELGSREDSPMSPSDTQDQKRTLRPPSRHGHSVVAPGRTAVRARMPALPRREGVDKPGFSLATQPRPGAPPSASASPAHHASTQGTSHRPSLPASLNDNDLVDSDEDERAVGSLHPKGAFAQPRPALSPSRQSPSSVLRDRSSVHPGAKPASPARRTPHSGAAEEDSSASAPPSRLSPPHGGSSRLLPTQPHLSSPLSKGGKDGEDAPATNSNAPSRSTMSSSVSSHLSSRTQVSEGAEASDGESHGDGDREDGGRQAEATAQTLRARPASGHFHLLRHKPFAANGRSPSRFSIGRGPRLQPSSSPQSTVPSRAHPRVPSHSDSHPKLSSGIHGDEEDEKPLPATVVNDHVPSSSRQPISRGWEDLRRSPQRGASLHRKEPIPENPKSTGADTHPQGKYSSLASKAQDVQQSTDADTEGHSPKAQPGSTDRHASPARPPAARSQQHPSVPRRMTPGRAPQQQPPPPVATSQHHPGPQSRDAGRSPSQPRLSLTQAGRPRPTSQGRSHSSSDPYTASSRGMLPTALQNQDEDAQGSYDDDSTEVEAQDVRAPAHAARAKEAAASLPKHQQVESPTGAGAGGDHRSQRGHAASPARPSRPGGPQSRARVPSRAAPGKSEPPSKRPLSSKSQQSVSAEDDEEEDAGFFKGGKEDLLSSSVPKWPSSSTPRGGKDADGSLAKEEREPAIALAPRGGSLAPVKRPLPPPPGSSPRASHVPSRLPPRSAATVSPVAGTHPWPQYTTRAPPGHFSTTPMLSLRQRMMHARFRNPLSRQPARPSYRQGYNGRPNVEGKVLPGSNGKPNGQRIINGPQGTKWVVDLDRGLVLNAEGRYLQDSHGNPLRIKLGGDGRTIVDLEGTPVVSPDGLPLFGQGRHGTPLANAQDKPILSLGGKPLVGLEVIKKTTHPPTTTMQPTTTTTPLPTTTTPRPTTATTRRTTTTRRTTTRRPTTTVRTTTRTTTTTTPTPTTPIPTCPPGTLERHDDDGNLIMSSNGIPECYAEEDEFSGLETDTAVPTEEAYVIYDEDYEFETSRPPTTTEPSTTATTPRVIPEEGAISSFPEEEFDLAGRKRFVAPYVTYLNKDPSAPCSLTDALDHFQVDSLDEIIPNDLKKSDLPPQHAPRNITVVAVEGCHSFVIVDWDKATPGDVVTGYLVYSASYEDFIRNKWSTQASSVTHLPIENLKPNTRYYFKVQAQNPHGYGPISPSVSFVTESDNPLLVVRPPGGEPIWIPFAFKHDPSYTDCHGRQYVKRTWYRKFVGVVLCNSLRYKIYLSDNLKDTFYSIGDSWGRGEDHCQFVDSHLDGRTGPQSYVEALPTIQGYYRQYRQEPVRFGNIGFGTPYYYVGWYECGVSIPGKW

SEQ ID NO: 203 - protein, 384 aa, Homo sapiens
MTNSSSTSTSSTTGGSLLLLCEEEESWAGRRIPVSLLYSGLAIGGTLANGMVIYLVSSFRKLQTTSNAFIVNGCAADLSVCALWMPQEAVLGLLPTGSAEPPADWDGAGGSYRLLRGGLLGLGLTVSLLSHCLVALNRYLLITRAPATYQALYQRRHTAGMLALSWALALGLVLLLPPWAPRPGAAPPRVHYPALLAAAALLAQTALLLHCYLGIVRRVRVSVKRVSVLNFHLLHQLPGCAAAAAAFPGAQHAPGPGGAAHPAQAQPLPPALHPRRAQRRLSGLSVLLLCCVFLLATQPLVWVSLASGFSLPVPWGVQAASWLLCCALSALNPLLYTWRNEEFRRSVRSVLPGVGDAAAAAVAATAVPAVSQAQLGTRAAGQHW

SEQ ID NO: 204 - protein, 317 aa, Homo sapiens
MKTALILLSILGMACAFSMKNLHRRVKIEDSEENGVFKYRPRYYLYKHAYFYPHLKRFPVQGSSDSSEENGDDSSEEEEEEEETSNEGENNEESNEDEDSEAENTTLSATTLGYGEDATPGTGYTGLAAIQLPKKAGDITNKATKEKESDEEEEEEEEGNENEESEAEVDENEQGINGTSTNSTEAENGNGSSGGDNGEEGEEESVTGANAEDTTETGRQGKGTSKTTTSPNGGFEPTTPPQVYRTTSPPFGKTTTVEYEGEYEYTGANEYDNGYEIYESENGEPRGDNYRAYEDEYSYFKGQGYDGYDGQNYYHHQ

SEQ ID NO: 205 - protein, 924 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 206 - protein, 938 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRSRQTQGALNESDDPETGCLTDNKPTSRHFYPVALLLVSSHLLVVWLILSLALLLAKYQ

SEQ ID NO: 207 - protein, 924 aa, Homo sapiens; identical to SEQ ID NO: 205

SEQ ID NO: 208 - protein, 938 aa, Homo sapiens; identical to SEQ ID NO: 206

SEQ ID NO: 209 - protein, 924 aa, Homo sapiens; identical to SEQ ID NO: 205

SEQ ID NO: 210 - protein, 720 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPRDISHFQEQNDLKALLENLLQNIQSKKRKNVEIMWLAATICRKLNGIRFTCCKSAKDRTSMSVTLEQCSILRDEHQLHKDFFIRALDCMRSRQTQGALNESDDPETGCLTDNKPTSRHFYPVALLLVSSHLLVVWLILSLALLLAKYQ

SEQ ID NO: 211 - protein, 924 aa, Homo sapiens; identical to SEQ ID NO: 205

SEQ ID NO: 212 - protein, 933 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDISSGIPQIDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 213 - protein, 706 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPRDISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 214 - protein, 924 aa, Homo sapiens; identical to SEQ ID NO: 205

SEQ ID NO: 215 - protein, 924 aa, Homo sapiens; identical to SEQ ID NO: 205

SEQ ID NO: 216 - protein, 933 aa, Homo sapiens; identical to SEQ ID NO: 212

SEQ ID NO: 217 - protein, 924 aa, Homo sapiens; identical to SEQ ID NO: 205

SEQ ID NO: 218 - protein, 1839 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREMEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRRLWYEDDAGLINELGRRIPSFSFDWNGFRRNGTSSCLYLWWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRRLWYEDDAGLINELGRRIPSFSFDWNGFRRNGTSSCLYLW

SEQ ID NO: 219 - protein, 920 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRRLWYEDDAGLINELGRRIPSFSFDWNGFRRNGTSSCLYLW

SEQ ID NO: 220 - protein, 739 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 221 - protein, 789 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDISSGIPQIDGVSLLLPRLECNGAISAHCNLCLLGSCDSPASASPVAEITDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 222 - protein, 671 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 223 - protein, 667 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRRLWYEDDAGLINELGRRIPSFSFDWNGFRRNGTSSCLYLW

SEQ ID NO: 224 - protein, 739 aa, Homo sapiens; identical to SEQ ID NO: 220

SEQ ID NO: 225 - protein, 710 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRRCDHLVAGKQAQGCH

SEQ ID NO: 226 - protein, 735 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRRMPHRECTEEYQMQKVCFQHATADGFPQVLQTSRGDLWKS

SEQ ID NO: 227 - protein, 747 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRSCTFSPFTREGCRIENVLKNIKCRKYAFNMLQLMAFPKYYRPPEGTYGKADT

SEQ ID NO: 228 - protein, 739 aa, Homo sapiens; identical to SEQ ID NO: 220

SEQ ID NO: 229 - protein, 680 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDISSGIPQIDALYDVITVGAPAAHFQGFKNGGLRKLLHRFETERRNTGYQFIYYSPENTAKAKEVLSNINQLQPLIATHADLLLNSASQHSPDSLKNSLKMLSEKDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 230 - protein, 676 aa, Homo sapiens
MRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 231 - protein, 521 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPRDISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 232 - protein, 748 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDISSGIPQIDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRREGCRIENVLKNIKCRKYAFNMLQLMAFPKYYRPPEGTYGKADT

SEQ ID NO: 233 - protein, 739 aa, Homo sapiens; identical to SEQ ID NO: 220

SEQ ID NO: 234 - protein, 671 aa, Homo sapiens; identical to SEQ ID NO: 222

SEQ ID NO: 235 - protein, 753 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRSRQTQGALNESDDPETGCLTDNKPTSRHFYPVALLLVSSHLLVVWLILSLALLLAKYQ

SEQ ID NO: 236 - protein, 809 aa, Homo sapiens
MIHFHPLILPHERSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRSRQTQGALNESDDPETGCLTDNKPTSRHFYPVALLLVSSHLLVVWLILSLALLLAKYQ

SEQ ID NO: 237 - protein, 739 aa, Homo sapiens; identical to SEQ ID NO: 220

SEQ ID NO: 238 - protein, 924 aa, Homo sapiens; identical to SEQ ID NO: 205

SEQ ID NO: 239 - protein, 947 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDISSGIPQIDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRSRQTQGALNESDDPETGCLTDNKPTSRHFYPVALLLVSSHLLVVWLILSLALLLAKYQ

SEQ ID NO: 240 - protein, 933 aa, Homo sapiens; identical to SEQ ID NO: 212

SEQ ID NO: 241 - protein, 879 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDISSGIPQIDALYDVITVGAPAAHFQGFKNGGLRKLLHRFETERRNTGYQFIYYSPENTAKAKEVLSNINQLQPLIATHADLLLNSASQHSPDSLKNSLKMLSEKDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRSRQTQGALNESDDPETGCLTDNKPTSRHFYPVALLLVSSHLLVVWLILSLALLLAKYQ

SEQ ID NO: 242 - protein, 870 aa, Homo sapiens
MEIKEEGASEEGQHFLPTAQANDPGDCQFTSIQKTPNEPQLEFILACKDLVAPVRDRKLNTLVQISVIHPVEQSLTRYSSTEIVEGTRDPLFLTGVTFPSEYPIYEETKIKLTVYDVKDKSHDTVRTSVLPEHKDPPPEVGRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDALYDVITVGAPAAHFQGFKNGGLRKLLHRFETERRNTGYQFIYYSPENTAKAKEVLSNINQLQPLIATHADLLLNSASQHSPDSLKNSLKMLSEKDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRSRQTQGALNESDDPETGCLTDNKPTSRHFYPVALLLVSSHLLVVWLILSLALLLAKYQ

SEQ ID NO: 243 - protein, 841 aa, Homo sapiens
MDKPTFCFHRSSWSGEQEKHSMTCGHKFYLQLCLMRSFLGYASFKVGELLKSKEQLLVLSLRTSDGGKVVGTIEVSVVKMGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDISSGIPQIDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRSRQTQGALNESDDPETGCLTDNKPTSRHFYPVALLLVSSHLLVVWLILSLALLLAKYQ

SEQ ID NO: 244 - protein, 762 aa, Homo sapiens
MGEIEDGEADHITTDVQGQKCALVCECTAPESVSGKDNLPFLNSVLKNPVCKLYRFPTSDNKWMRIREQMSESILSFHIPKELISLHIKEDLCRNQEIKELGELSPHWDNLRKNVLTHCDQMVNMYQDILTELSKETGSSFKSSSSKGEKTLEFVPINLHLQRMQVHSPHLKDISSGIPQIDALYDVITVGAPAAHFQGFKNGGLRKLLHRFETERRNTGYQFIYYSPENTAKAKEVLSNINQLQPLIATHADLLLNSASQHSPDSLKNSLKMLSEKTELFVHAFKDQLVRSALLALYTARPGGILKKPPSPKSSTEESSPQDQPPVMRGQDSIPHHSDYDEEEWDRVWANVGKSLNCIIAMVDKLIERDGGSEGSGGNNDGEKEPSLTDAIPSHPREDWYEQLYPLILTLKDCMGEVVNRAKQSLTFVLLQELAYSLPQCLMLTLRRDIVFSQALAGLVCGFIIKLQTSLYDPGFLQQLHTVGLIVQYEGLLSTYSDEIGMLEDMAVGISDLKKVAFKIIEAKSNDVLPVITGRREHYVVEVKLPARMFESLPLQIKEGQLLHVYPVLFNVGINEQQTLAERFGDVSLQESINQENFELLQEYYKIFMEKMPPDYISHFQEQNDLKALLENLLQNIQSKKRKNVEIMWLAATICRKLNGIRFTCCKSAKDRTSMSVTLEQCSILRDEHQLHKDFFIRALDCMRSRQTQGALNESDDPETGCLTDNKPTSRHFYPVALLLVSSHLLVVWLILSLALLLAKYQ

SEQ ID NO: 245 - protein, 947 aa, Homo sapiens; identical to SEQ ID NO: 239

SEQ ID NO: 246 - protein, 938 aa, Homo sapiens; identical to SEQ ID NO: 206

SEQ ID NO: 247 - protein, 464 aa, Homo sapiens
MVRLAAELLLLLGLLLLTLHITVLRGSGAADGPDAAAGNASQAQLQNNLNVGSDTTSETSFSLSKEAPREHLDHQAAHQPFPRPRFRQETGHPSLQRDFPRSFLLDLPNFPDLSKADINGQNPNIQVTIEVVDGPDSEADKDQHPENKPSWSVPSPDWRAWWQRSLSLARANSGDQDYKYDSTSDDSNFLNPPRGWDHTAPGHRTFETKDQPEYDSTDGEGDWSLWSVCSVTCGNGNQKRTRSCGYACTATESRTCDRPNCPGIEDTFRTAATEVSLLAGSEEFNATKLFEVDTDSCERWMSCKSEFLKKYMHKVMNDLPSCPCSYPTEVAYSTADIFDRIKRKDFRWKDASGPKEKLEIYKPTARYCIRSMLSLESTTLAAQHCCYGDNMQLITRGKGAGTPNLISTEFSAELHYKVDVLPWIICKGDWSRYNEARPPNNGQKCTESPSDEDYIKQFQEAREY

SEQ ID NO: 248 - protein, 400 aa, Homo sapiens
MVRLAAELLLLLGLLLLTLHITVLRGSGAADGPDAAAGNASQAQLQNNLNVGSDTTSETSFSLSKEAPREHLDHQAAHQPFPRPRFRQETGHPSLQRDFPRSFLLDLPNFPDLSKADINGQNPNIQVTIEVVDGPDSEADKDQHPENKPSWSVPSPDWRAWWQRSLSLARANSGDQDYKYDSTSDDSNFLNPPRGWDHTAPGHRTFETKDQPEYDSTDGEGDWSLWSVCSVTCGNGNQKRTRSCGYACTATESRTCDRPNCPGIEDTFRTAATEVSLLAGSEEFNATKLFEVGSVPTRELLDARNREPLDLSDHQRVPTGQPDKWEWLYHFHLQHQYVSVPYIHILSCYTSSPTLGMNFFYFSHSNRYIVVSQHVVSLISLAFHDVEHFSCVSLPFINPF

SEQ ID NO: 249 - protein, 1188 aa, Homo sapiens
MDLPRGLVVAWALSLWPGFTDTFNMDTRKPRVIPGSRTAFFGYTVQQHDISGNKWLVVGAPLETNGYQKTGDVYKCPVIHGNCTKLNLGRVTLSNVSERKDNMRLGLSLATNPKDNSFLACSPLWSHECGSSYYTTGMCSRVNSNFRFSKTVAPALQRCQTYMDIVIVLDGSNSIYPWVEVQHFLINILKKFYIGPGQIQVGVVQYGEDVVHEFHLNDYRSVKDVVEAASHIEQRGGTETRTAFGIEFARSEAFQKGGRKGAKKVMIVITDGESHDSPDLEKVIQQSERDNVTRYAVAVLGYYNRRGINPETFLNEIKYIASDPDDKHFFNVTDEAALKDIVDALGDRIFSLEGTNKNETSFGLEMSQTGFSSHVVEDGVLLGAVGAYDWNGAVLKETSAGKVIPLRESYLKEFPEELKNHGAYLGYTVTSVVSSRQGRVYVAGAPRFNHTGKVILFTMHNNRSLTIHQAMRGQQIGSYFGSEITSVDIDGDGVTDVLLVGAPMYFNEGRERGKVYVYELRQNLFVYNGTLKDSHSYQNARFGSSIASVRDLNQDSYNDVVVGAPLEDNHAGAIYIFHGFRGSILKTPKQRITASELATGLQYFGCSIHGQLDLNEDGLIDLAVGALGNAVILWSRPVVQINASLHFEPSKINIFHRDCKRSGRDATCLAAFLCFTPIFLAPHFQTTTVGIRYNATMDERRYTPRAHLDEGGDRFTNRAVLLSSGQELCERINFHVLDTADYVKPVTFSVEYSLEDPDHGPMLDDGWPTTLRVSVPFWNGCNEDEHCVPDLVLDARSDLPTAMEYCQRVLRKPAQDCSAYTLSFDTTVFIIESTRQRVAVEATLENRGENAYSTVLNISQSANLQFASLIQKEDSDGSIECVNEERRLQKQVCNVSYPFFRAKAKVAFRLDFEFSKSIFLHHLEIELAAGSDSNERDSTKEDNVAPLRFHLKYEADVLFTRSSSLSHYEVKPNSSLERYDGIGPPFSCIFRIQNLGLFPIHGMMMKITIPIATRSGNRLLKLRDFLTDEANTSCNIWGNSTEYRPTPVEEDLRRAPQLNHSNSDVVSINCNIRLVPNQEINFHLLGNLWLRSLKALKYKSMKIMVNAALQRQFHSPFIFREEDPSRQIVFEISKQEDWQVPIWIIVGSTLGGLLLLALLVLALWKLGFFRSARRRREPGLDPTPKVLE

SEQ ID NO: 250 - protein, 1086 aa, Homo sapiens
MRLGLSLATNPKDNSFLACSPLWSHECGSSYYTTGMCSRVNSNFRFSKTVAPALQRCQTYMDIVIVLDGSNSIYPWVEVQHFLINILKKFYIGPGQIQVGVVQYGEDVVHEFHLNDYRSVKDVVEAASHIEQRGGTETRTAFGIEFARSEAFQKGGRKGAKKVMIVITDGESHDSPDLEKVIQQSERDNVTRYAVAVLGYYNRRGINPETFLNEIKYIASDPDDKHFFNVTDEAALKDIVDALGDRIFSLEGTNKNETSFGLEMSQTGFSSHVVEDGVLLGAVGAYDWNGAVLKETSAGKVIPLRESYLKEFPEELKNHGAYLGYTVTSVVSSRQGRVYVAGAPRFNHTGKVILFTMHNNRSLTIHQAMRGQQIGSYFGSEITSVDIDGDGVTDVLLVGAPMYFNEGRERGKVYVYELRQNLFVYNGTLKDSHSYQNARFGSSIASVRDLNQDSYNDVVVGAPLEDNHAGAIYIFHGFRGSILKTPKQRITASELATGLQYFGCSIHGQLDLNEDGLIDLAVGALGNAVILWSRPVVQINASLHFEPSKINIFHRDCKRSGRDATCLAAFLCFTPIFLAPHFQTTTVGIRYNATMDERRYTPRAHLDEGGDRFTNRAVLLSSGQELCERINFHVLDTADYVKPVTFSVEYSLEDPDHGPMLDDGWPTTLRVSVPFWNGCNEDEHCVPDLVLDARSDLPTAMEYCQRVLRKPAQDCSAYTLSFDTTVFIIESTRQRVAVEATLENRGENAYSTVLNISQSANLQFASLIQKEDSDGSIECVNEERRLQKQVCNVSYPFFRAKAKVAFRLDFEFSKSIFLHHLEIELAAGSDSNERDSTKEDNVAPLRFHLKYEADVLFTRSSSLSHYEVKPNSSLERYDGIGPPFSCIFRIQNLGLFPIHGMMMKITIPIATRSGNRLLKLRDFLTDEANTSCNIWGNSTEYRPTPVEEDLRRAPQLNHSNSDVVSINCNIRLVPNQEINFHLLGNLWLRSLKALKYKSMKIMVNAALQRQFHSPFIFREEDPSRQIVFEISKQEDWQVPIWIIVGSTLGGLLLLALLVLALWKLGFFRSARRRREPGLDPTPKVLE

SEQ ID NO: 251 - protein, 1119 aa, Homo sapiens
MDLPRGLVVAWALSLWPGFTDTFNMDTRKPRVIPGSRTAFFGYTVQQHDISGNKWLVVGAPLETNGYQKTGDVYKCPVIHGNCTKLNLGCQTYMDIVIVLDGSNSIYPWVEVQHFLINILKKFYIGPGQIQVGVVQYGEDVVHEFHLNDYRSVKDVVEAASHIEQRGGTETRTAFGIEFARSEAFQKGGRKGAKKVMIVITDGESHDSPDLEKVIQQSERDNVTRYAVAVLGYYNRRGINPETFLNEIKYIASDPDDKHFFNVTDEAALKDIVDALGDRIFSLEGTNKNETSFGLEMSQTGFSSHVVEDGVLLGAVGAYDWNGAVLKETSAGKVIPLRESYLKEFPEELKNHGAYLGYTVTSVVSSRQGRVYVAGAPRFNHTGKVILFTMHNNRSLTIHQAMRGQQIGSYFGSEITSVDIDGDGVTDVLLVGAPMYFNEGRERGKVYVYELRQNLFVYNGTLKDSHSYQNARFGSSIASVRDLNQDSYNDVVVGAPLEDNHAGAIYIFHGFRGSILKTPKQRITASELATGLQYFGCSIHGQLDLNEDGLIDLAVGALGNAVILWSRPVVQINASLHFEPSKINIFHRDCKRSGRDATCLAAFLCFTPIFLAPHFQTTTVGIRYNATMDERRYTPRAHLDEGGDRFTNRAVLLSSGQELCERINFHVLDTADYVKPVTFSVEYSLEDPDHGPMLDDGWPTTLRVSVPFWNGCNEDEHCVPDLVLDARSDLPTAMEYCQRVLRKPAQDCSAYTLSFDTTVFIIESTRQRVAVEATLENRGENAYSTVLNISQSANLQFASLIQKEDSDGSIECVNEERRLQKQVCNVSYPFFRAKAKVAFRLDFEFSKSIFLHHLEIELAAGSDSNERDSTKEDNVAPLRFHLKYEADVLFTRSSSLSHYEVKPNSSLERYDGIGPPFSCIFRIQNLGLFPIHGMMMKITIPIATRSGNRLLKLRDFLTDEANTSCNIWGNSTEYRPTPVEEDLRRAPQLNHSNSDVVSINCNIRLVPNQEINFHLLGNLWLRSLKALKYKSMKIMVNAALQRQFHSPFIFREEDPSRQIVFEISKQEDWQVPIWIIVGSTLGGLLLLALLVLALWKLGFFRSARRRREPGLDPTPKVLE

SEQ ID NO: 252 - protein, 399 aa, Homo sapiens
MWQLLAAACWMLLLGSMYGYDKKGNNANPEANMNISQIISYWGYPYEEYDVTTKDGYILGIYRIPHGRGCPGRTAPKPAVYLQHGLIASASNWICNLPNNSLAFLLADSGYDVWLGNSRGNTWSRKHLKLSPKSPEYWAFSLDEMAKYDLPATINFIIEKTGQKRLYYVGHSQGTTIAFIAFSTNPELAKKIKIFFALAPVVTVKYTQSPMKKLTTLSRRVVKVLFGDKMFHPHTLFDQFIATKVCNRKLFRRICSNFLFTLSGFDPQNLNMSRLDVYLSHNPAGTSVQNMLHWAQAVNSGQLQAFDWGNSDQNMMHFHQLTPPLYNITKMEVPTAIWNGGQDIVADPKDVENLLPQIANLIYYKLIPHYNHVDFYLGEDAPQEIYQDLIILMEEYLQN

SEQ ID NO: 253 - protein, 366 aa, Homo sapiens
MWQLLAAACWMLLLGSMYGYDKKGNNANPEANMNISQIISYWGYPYEEYDVTTKDGYILGIYRIPHGRGCPGRTDSGYDVWLGNSRGNTWSRKHLKLSPKSPEYWAFSLDEMAKYDLPATINFIIEKTGQKRLYYVGHSQGTTIAFIAFSTNPELAKKIKIFFALAPVVTVKYTQSPMKKLTTLSRRVVKVLFGDKMFHPHTLFDQFIATKVCNRKLFRRICSNFLFTLSGFDPQNLNMSRLDVYLSHNPAGTSVQNMLHWAQAVNSGQLQAFDWGNSDQNMMHFHQLTPPLYNITKMEVPTAIWNGGQDIVADPKDVENLLPQIANLIYYKLIPHYNHVDFYLGEDAPQEIYQDLIILMEEYLQN

SEQ ID NO: 254 - protein, 419 aa, Homo sapiens
MHLKLPGLEDRPPKTWIRSQMWQLLAAACWMLLLGSMYGYDKKGNNANPEANMNISQIISYWGYPYEEYDVTTKDGYILGIYRIPHGRGCPGRTAPKPAVYLQHGLIASASNWICNLPNNSLAFLLADSGYDVWLGNSRGNTWSRKHLKLSPKSPEYWAFSLDEMAKYDLPATINFIIEKTGQKRLYYVGHSQGTTIAFIAFSTNPELAKKIKIFFALAPVVTVKYTQSPMKKLTTLSRRVVKVLFGDKMFHPHTLFDQFIATKVCNRKLFRRICSNFLFTLSGFDPQNLNMSRLDVYLSHNPAGTSVQNMLHWAQAVNSGQLQAFDWGNSDQNMMHFHQLTPPLYNITKMEVPTAIWNGGQDIVADPKDVENLLPQIANLIYYKLIPHYNHVDFYLGEDAPQEIYQDLIILMEEYLQN

SEQ ID NO: 255 - protein, 408 aa, Homo sapiens
MESLLGRSQMWQLLAAACWMLLLGSMYGYDKKGNNANPEANMNISQIISYWGYPYEEYDVTTKDGYILGIYRIPHGRGCPGRTAPKPAVYLQHGLIASASNWICNLPNNSLAFLLADSGYDVWLGNSRGNTWSRKHLKLSPKSPEYWAFSLDEMAKYDLPATINFIIEKTGQKRLYYVGHSQGTTIAFIAFSTNPELAKKIKIFFALAPVVTVKYTQSPMKKLTTLSRRVVKVLFGDKMFHPHTLFDQFIATKVCNRKLFRRICSNFLFTLSGFDPQNLNMSRLDVYLSHNPAGTSVQNMLHWAQAVNSGQLQAFDWGNSDQNMMHFHQLTPPLYNITKMEVPTAIWNGGQDIVADPKDVENLLPQIANLIYYKLIPHYNHVDFYLGEDAPQEIYQDLIILMEEYLQN

SEQ ID NO: 256 - protein, 265 aa, Homo sapiens
MTRKFPSLDEMAKYDLPATINFIIEKTGQKRLYYVGHSQGTTIAFIAFSTNPELAKKIKIFFALAPVVTVKYTQSPMKKLTTLSRRVVKVLFGDKMFHPHTLFDQFIATKVCNRKLFRRICSNFLFTLSGFDPQNLNMSRLDVYLSHNPAGTSVQNMLHWAQAVNSGQLQAFDWGNSDQNMMHFHQLTPPLYNITKMEVPTAIWNGGQDIVADPKDVENLLPQIANLIYYKLIPHYNHVDFYLGEDAPQEIYQDLIILMEEYLQN

SEQ ID NO: 257 - protein, 395 aa, Homo sapiens
MHLKLPGLEDRPPKTWIRSQMWQLLAAACWMLLLGSMYGYDKKGNNANPEANMNISQIISYWGYPYEEYDVTTKDGYILGIYRIPHGRGCPGRTAPKPAVYLQHGLIASASNWICNLPNNSLAFLLADSGYDVWLGNSRGNTWSRKHLKLSPKSPEYWAFSLDEMAKYDLPATINFIIEKTGQKRLYYVGHSQGTTIAFIAFSTNPELAKKIKIFFALAPVVTVKYTQSPMKKLTTLSRRVVKVLFGDKMFHPHTLFDQFIATKVCNRKLFRRICSNFLFTLSGFDPQNLNMSRLDVYLSHNPAGTSVQNMLHWAQLTPPLYNITKMEVPTAIWNGGQDIVADPKDVENLLPQIANLIYYKLIPHYNHVDFYLGEDAPQEIYQDLIILMEEYLQN

SEQ ID NO: 258 - protein, 386 aa, Homo sapiens
MHLKLPGLEDRPPKTWIRSQMWQLLAAACWMLLLGSMYGYDKKGNNANPEANMNISQIISYWGYPYEEYDVTTKDGYILGIYRIPHGRGCPGRTDSGYDVWLGNSRGNTWSRKHLKLSPKSPEYWAFSLDEMAKYDLPATINFIIEKTGQKRLYYVGHSQGTTIAFIAFSTNPELAKKIKIFFALAPVVTVKYTQSPMKKLTTLSRRVVKVLFGDKMFHPHTLFDQFIATKVCNRKLFRRICSNFLFTLSGFDPQNLNMSRLDVYLSHNPAGTSVQNMLHWAQAVNSGQLQAFDWGNSDQNMMHFHQLTPPLYNITKMEVPTAIWNGGQDIVADPKDVENLLPQIANLIYYKLIPHYNHVDFYLGEDAPQEIYQDLIILMEEYLQN

SEQ ID NO: 259 - protein, 370 aa, Homo sapiens
MHLKLPGLEDRPPKTWIRSQMWQLLAAACWMLLLGSMYGYDKKGNNANPEANMNISQIISYWGYPYEEYDVTTKDGYILGIYRIPHGRGCPGRTAPKPAVYLQHGLIASASNWICNLPNNSLAFLLADSGYDVWLGNSRGNTWSRKHLKLSPKSPEYWAFSLDEMAKYDLPATINFIIEKTGQKRLYYVGHSQGTTIAFIAFSTNPELAKKIKIFFALAPVVTVKYTQSPMKKLTTLSRRVVKSRLDVYLSHNPAGTSVQNMLHWAQAVNSGQLQAFDWGNSDQNMMHFHQLTPPLYNITKMEVPTAIWNGGQDIVADPKDVENLLPQIANLIYYKLIPHYNHVDFYLGEDAPQEIYQDLIILMEEYLQN

SEQ ID NO: 260 - protein, 358 aa, Homo sapiens
MHLKLPGLEDRPPKTWIRSQMWQLLAAACWMLLLGSMYGYDKKGNNANPEANMNISQIISYWGYPYEEYDVTTKDGYILGIYRIPHGRGCPGRTAPKPAVYLQHGLIASASNWICNLPNNSLAFLLADSGYDVWLGNSRGNTWSRKHLKLSPKSPEYWAFSLDEMAKYDLPATINFIIEKTGQKRLYYVGHSQGTTIAFIAFSTNPELAKKIKIFFALAPVVTVKYTQSPMKKLTTLSRRVVKVLFGDKMFHPHTLFDQFIATKVCNRKLFRRICSNFLFTLSGFDPQNLNMSRLDVYLSHNPAGTSVQNMLHWAQKVISSNRTLYKMMHLIPERAVHPPINIWGISECGNDALFRED

SEQ ID NO: 261 - protein, 522 aa, Homo sapiens
MDFLLLGLCLYWLLRRPSGVVLCLLGACFQMLPAAPSGCPQLCRCEGRLLYCEALNLTEAPHNLSGLLGLSLRYNSLSELRAGQFTGLMQLTWLYLDHNHICSVQGDAFQKLRRVKELTLSSNQITQLPNTTFRPMPNLRSVDLSYNKLQALAPDLFHGLRKLTTLHMRANAIQFVPVRIFQDCRSLKFLDIGYNQLKSLARNSFAGLFKLTELHLEHNDLVKVNFAHFPRLISLHSLCLRRNKVAIVVSSLDWVWNLEKMDLSGNEIEYMEPHVFETVPHLQSLQLDSNRLTYIEPRILNSWKSLTSITLAGNLWDCGRNVCALASWLNNFQGRYDGNLQCASPEYAQGEDVLDAVYAFHLCEDGAEPTSGHLLSAVTNRSDLGPPASSATTLADGGEGQHDGTFEPATVALPGGEHAENAVQIHKVVTGTMALIFSFLIVVLVLYVSWKCFPASLRQLRQCFVTQRRKQKQKQTMHQMAAMSAQEYYVDYKPNHIEGALVIINEYGSCTCHQQPARECEV

SEQ ID NO: 262 - protein, 522 aa, Homo sapiens; identical to SEQ ID NO: 261

SEQ ID NO: 263 - protein, 522 aa, Homo sapiens; identical to SEQ ID NO: 261

SEQ ID NO: 264 - protein, 360 aa, Homo sapiens [sequence omitted]

SEQ ID NO: 265 - protein, 360 aa, Homo sapiens [sequence omitted]

SEQ ID NO: 266 - protein, 486 aa, Homo sapiens
MPRPAPARRLPGLLLLLWPLLLLPSAAPDPVARPGFRRLETRGPGGSPGRRPSPAAPDGAPASGTSEPGRARGAGVCKSRPLDLVFIIDSSRSVRPLEFTKVKTFVSRIIDTLDIGPADTRVAVVNYASTVKIEFQLQAYTDKQSLKQAVGRITPLSTGTMSGLAIQTAMDEAFTVEAGAREPSSNIPKVAIIVTDGRPQDQVNEVAARAQASGIELYAVGVDRADMASLKMMASEPLEEHVFYVETYGVIEKLSSRFQETFCALDPCVLGTHQCQHVCISDGEGKHHCECSQGYTLNADKKTCSALDRCALNTHGCEHICVNDRSGSYHCECYEGYTLNEDRKTCSAQDKCALGTHGCQHICVNDRTGSHHCECYEGYTLNADKKTCSVRDKCALGSHGCQHICVSDGAASYHCDCYPGYTLNEDKKTCSATEEARRLVSTEDACGCEATLAFQDKVSSYLQRLNTKLDDILEKLKINEYGQIHR

SEQ ID NO: 267 - protein, 163 aa, Homo sapiens
MSLLGPKVLLFLAAFIITSDWIPLGVNSQRGDDVTQATPETFTEDPNLVNDPATDETVLAVLADIAPSTDDLECWDEKFTCTRLYSVHRPVKQCIHQLCFTSLRRMYIVNKEICSRLVCKEHEAMKDELCRQMAGLPPRRLRRSNYFRLPPCENVDLQRPNGL

SEQ ID NO: 268 - protein, 151 aa, Homo sapiens
MSLLGPKVLLFLAAFIITSDDVTQATPETFTEDPNLVNDPATDETVLAVLADIAPSTDDLECWDEKFTCTRLYSVHRPVKQCIHQLCFTSLRRMYIVNKEICSRLVCKEHEAMKDELCRQMAGLPPRRLRRSNYFRLPPCENVDLQRPNGL

SEQ ID NO: 269 - protein, 148 aa, Homo sapiens
MSLLGPKVLLFLAAFIITSDWIPLGVNSQRGDDVTQATPETFTEDPNLVNDPATDETECWDEKFTCTRLYSVHRPVKQCIHQLCFTSLRRMYIVNKEICSRLVCKEHEAMKDELCRQMAGLPPRRLRRSNYFRLPPCENVDLQRPNGL

SEQ ID NO: 270 - protein, 109 aa, Homo sapiens
MSLLGPKVLLFLAAFIITSDWIPLGVNSQRGDDVTQATPETFTEDPNLVNDPATDETVLAVLADIAPSTDDLDELCRQMAGLPPRRLRRSNYFRLPPCENVDLQRPNGL

SEQ ID NO: 271 - protein, 173 aa, Homo sapiens
MSLLGPKVLLFLAAFIITSDWIPLGVNSQRGDDVTQATPETFTEDPNLVNDPATDETVLAVLADIAPSTDDLASLSEKNTTAECWDEKFTCTRLYSVHRPVKQCIHQLCFTSLRRMYIVNKEICSRLVCKEHEAMKDELCRQMAGLPPRRLRRSNYFRLPPCENVDLQRPNGL

SEQ ID NO: 272 - protein, 488 aa, Homo sapiens
MAPAAWLRSAAARALLPPMLLLLLQPPPLLARALPPDAHHLHAERRGPQPWHAALPSSPAPAPATQEAPRPASSLRPPRCGVPDPSDGLSARNRQKRFVLSGGRWEKTDLTYRILRFPWQLVQEQVRQTMAEALKVWSDVTPLTFTEVHEGRADIMIDFARYWHGDDLPFDGPGGILAHAFFPKTHREGDVHFDYDETWTIGDDQGTDLLQVAAHEFGHVLGLQHTTAAKALMSAFYTFRYPLSLSPDDCRGVQHLYGQPWPTVTSRTPALGPQAGIDTNEIAPLEPDAPPDACEASFDAVSTIRGELFFFKAGFVWRLRGGQLQPGYPALASRHWQGLPSPVDAAFEDAQGHIWFFQGAQYWVYDGEKPVLGPAPLTELGLVRFPVHAALVWGPEKNKIYFFRGRDYWRFHPSTRRVDSPVPRRATDWRGVPSEIDAAFQDADGYAYFLRGRLYWKFDPVKVKALEGFPRLVGPDFFGCAEPANTFL

SEQ ID NO: 273 - protein, 1314 aa, Homo sapiens
MKHLYGLFHYNPMMLGLESLPDPTDTWEIIETIGKGTYGKVYKVTNKRDGSLAAVKILDPVSDMDEEIEAEYNILQFLPNHPNVVKFYGMFYKADHCVGGQLWLVLELCNGGSVTELVKGLLRCGQRLDEAMISYILYGALLGLQHLHNNRIIHRDVKGNNILLTTEGGVKLVDFGVSAQLTSTRLRRNTSVGTPFWMAPEVIACEQQYDSSYDARCDVWSLGITAIELGDGDPPLFDMHPVKTLFKIPRNPPPTLLHPEKWCEEFNHFISQCLIKDFERRPSVTHLLDHPFIKGVHGKVLFLQKQLAKVLQDQKHQNPVAKTRHERMHTRRPYHVEDAEKYCLEDDLVNLEVLDEDTIIHQLQKRYADLLIYTYVGDILIALNPFQNLSIYSPQFSRLYHGVKRASNPPHIFASADAAYQCMVTLSKDQCIVISGESGSGKTESAHLIVQHLTFLGKANNQTLREKILQVNSLVEAFGNSCTAINDNSSRFGKYLEMMFTPTGVVMGARISEYLLEKSRVIKQAAREKNFHIFYYIYAGLHHQKKLSDFRLPEEKPPRYIADETGRVMHDITSKESYRRQFEAIQHCFRIIGFTDKEVHSVYRILAGILNIGNIEFAAISSQHQTDKSEVPNAEALQNAASVLCISPEELQEALTSHCVVTRGETIIRANTVDRAADVRDAMSKALYGRLFSWIVNRINTLLQPDENICSAGGGMNVGILDIFGFENFQRNSFEQLCINIANEQIQYYFNQHVFALEQMEYQNEGIDAVPVEYEDNRPLLDMFLQKPLGLLALLDEESRFPQATDQTLVDKFEDNLRCKYFWRPKGVELCFGIQHYAGKVLYDASGVLEKNRDTLPADVVVVLRTSENKLLQQLFSIPLTKTGNLAQTRARITVASSSLPPHFSAGKAKVDTLEVIRHPEETTNMKRQTVASYFRYSLMDLLSKMVVGQPHFVRCIKPNDDREALQFSRERVLAQLRSTGILETVSIRRQGYSHRILFEEFVKRYYYLAFTAHQTPLASKESCVAILEKSRLDHWVLGKTKVFLKYYHVEQLNLLLREVIGRVVVLQAYTKGWLGARRYKRVREKREKGAIAIQSGDTSNQSSGPHSPVAAGTRGSAEVQDCSEPGDHKVLRGSVHRRSHSQAESNNGRTQTSSNSPAVTEKNGHSQAQSSPKGCDIFAGHANKHSVSGTDLLSSRICHPAPDQQGLSLWGAPQKPGSENGLAQKHRTPRRRCQQPKMLSSPEDTMYYNQLNGTLEYQGSKRKPRKLGQIKVLDGEDEYYKSLSPVDCIPEENNSAHPSFFSSSSKGDSFAQH

SEQ ID NO: 274 - protein, 1341 aa, Homo sapiens
MKHLYGLFHYNPMMLGLESLPDPTDTWEIIETIGKGTYGKVYKVTNKRDGSLAAVKILDPVSDMDEEIEAEYNILQFLPNHPNVVKFYGMFYKADHCVGGQLWLVLELCNGGSVTELVKGLLRCGQRLDEAMISYILYGALLGLQHLHNNRIIHRDVKGNNILLTTEGGVKLVDFGVSAQLTSTRLRRNTSVGTPFWMAPEVIACEQQYDSSYDARCDVWSLGITAIELGDGDPPLFDMHPVKTLFKIPRNPPPTLLHPEKWCEEFNHFISQCLIKDFERRPSVTHLLDHPFIKGVHGKVLFLQKQLAKVLQDQKHQNPVAKTRHERMHTRRPYHVEDAEKYCLEDDLVNLEVLDEDTIIHQLQKRYADLLIYTYVGDILIALNPFQNLSIYSPQFSRLYHGVKRASNPPHIFASADAAYQCMVTLSKDQCIVISGESGSGKTESAHLIVQHLTFLGKANNQTLREKILQVNSLVEAFGNSCTAINDNSSRFGKYLEMMFTPTGVVMGARISEYLLEKSRVIKQAAREKNFHIFYYIYAGLHHQKKLSDFRLPEEKPPRYIADETGRVMHDITSKESYRRQFEAIQHCFRIIGFTDKEVHSVYRILAGILNIGNIEFAAISSQHQTDKSEVPNAEALQNAASVLCISPEELQEALTSHCVVTRGETIIRANTVDRAADVRDAMSKALYGRLFSWIVNRINTLLQPDENICSAGGGMNVGILDIFGFENFQRNSFEQLCINIANEQIQYYFNQHVFALEQMEYQNEGIDAVPVEYEDNRPLLDMFLQKPLGLLALLDEESRFPQATDQTLVDKFEDNLRCKYFWRPKGVELCFGIQHYAGKVLYDASGVLEKNRDTLPADVVVVLRTSENKLLQQLFSIPLTKTGNLAQTRARITVASSSLPPHFSAGKAKVDTLEVIRHPEETTNMKRQTVASYFRYSLMDLLSKMVVGQPHFVRCIKPNDDREALQFSRERVLAQLRSTGILETVSIRRQGYSHRILFEEFVKRYYYLAFTAHQTPLASKESCVAILEKSRLDHWVLGKTKVFLKYYHVEQLNLLLREVIGRVVVLQAYTKGWLGARRYKRVREKREKGAIAIQSAWRGYDARRKFKKISNRRNESAAHNQAGDTSNQSSGPHSPVAAGTRGSAEVQDCSEPGDHKVLRGSVHRRSHSQAESNNGRTQTSSNSPAVTEKNGHSQAQSSPKGCDIFAGHANKHSVSGTDLLSSRICHPAPDQQGLSLWGAPQKPGSENGLAQKHRTPRRRCQQPKMLSSPEDTMYYNQLNGTLEYQGSKRKPRKLGQIKVLDGEDEYYKSLSPVDCIPEENNSAHPSFFSSSSKGDSFAQH

SEQ ID NO: 275 - protein, 1350 aa, Homo sapiens
MLQSALLSTRKHLYGLFHYNPMMLGLESLPDPTDTWEIIETIGKGTYGKVYKVTNKRDGSLAAVKILDPVSDMDEEIEAEYNILQFLPNHPNVVKFYGMFYKADHCVGGQLWLVLELCNGGSVTELVKGLLRCGQRLDEAMISYILYGALLGLQHLHNNRIIHRDVKGNNILLTTEGGVKLVDFGVSAQLTSTRLRRNTSVGTPFWMAPEVIACEQQYDSSYDARCDVWSLGITAIELGDGDPPLFDMHPVKTLFKIPRNPPPTLLHPEKWCEEFNHFISQCLIKDFERRPSVTHLLDHPFIKGVHGKVLFLQKQLAKVLQDQKHQNPVAKTRHERMHTRRPYHVEDAEKYCLEDDLVNLEVLDEDTIIHQLQKRYADLLIYTYVGDILIALNPFQNLSIYSPQFSRLYHGVKRASNPPHIFASADAAYQCMVTLSKDQCIVISGESGSGKTESAHLIVQHLTFLGKANNQTLREKILQVNSLVEAFGNSCTAINDNSSRFGKYLEMMFTPTGVVMGARISEYLLEKSRVIKQAAREKNFHIFYYIYAGLHHQKKLSDFRLPEEKPPRYIADETGRVMHDITSKESYRRQFEAIQHCFRIIGFTDKEVHSVYRILAGILNIGNIEFAAISSQHQTDKSEVPNAEALQNAASVLCISPEELQEALTSHCVVTRGETIIRANTVDRAADVRDAMSKALYGRLFSWIVNRINTLLQPDENICSAGGGMNVGILDIFGFENFQRNSFEQLCINIANEQIQYYFNQHVFALEQMEYQNEGIDAVPVEYEDNRPLLDMFLQKPLGLLALLDEESRFPQATDQTLVDKFEDNLRCKYFWRPKGVELCFGIQHYAGKVLYDASGVLEKNRDTLPADVVVVLRTSENKLLQQLFSIPLTKTGNLAQTRARITVASSSLPPHFSAGKAKVDTLEVIRHPEETTNMKRQTVASYFRYSLMDLLSKMVVGQPHFVRCIKPNDDREALQFSRERVLAQLRSTGILETVSIRRQGYSHRILFEEFVKRYYYLAFTAHQTPLASKESCVAILEKSRLDHWVLGKTKVFLKYYHVEQLNLLLREVIGRVVVLQAYTKGWLGARRYKRVREKREKGAIAIQSAWRGYDARRKFKKISNRRNESAAHNQAGDTSNQSSGPHSPVAAGTRGSAEVQDCSEPGDHKVLRGSVHRRSHSQAESNNGRTQTSSNSPAVTEKNGHSQAQSSPKGCDIFAGHANKHSVSGTDLLSSRICHPAPDQQGLSLWGAPQKPGSENGLAQKHRTPRRRCQQPKMLSSPEDTMYYNQLNGTLEYQGSKRKPRKLGQIKVLDGEDEYYKSLSPVDCIPEENNSAHPSFFSSSSKGDSFAQH

SEQ ID NO: 276 - protein, 1386 aa, Homo sapiens
MLQSALLSTRKHLYGLFHYNPMMLGLESLPDPTDTWEIIETIGKGTYGKVYKVTNKRDGSLAAVKILDPVSDMDEEIEAEYNILQFLPNHPNVVKFYGMFYKADHCVGGQLWLVLELCNGGSVTELVKGLLRCGQRLDEAMISYILYGALLGLQHLHNNRIIHRDVKGNNILLTTEGGVKLVDFGVSAQLTSTRLRRNTSVGTPFWMAPEVIACEQQYDSSYDARCDVWSLGITAIELGDGDPPLFDMHPVKTLFKIPRNPPPTLLHPEKWCEEFNHFISQCLIKDFERRPSVTHLLDHPFIKGVHGKVLFLQKQLAKVLQDQKHQNPVAKTRHERMHTRRPYHVEDAEKYCLEDDLVNLEVLDEDTIIHQLQKRYADLLIYTYVGDILIALNPFQNLSIYSPQFSRLYHGVKRASNPPHIFASADAAYQCMVTLSKDQCIVISGESGSGKTESAHLIVQHLTFLGKANNQTLREKILQVNSLVEAFGNSCTAINDNSSRFGKYLEMMFTPTGVVMGARISEYLLEKSRVIKQAAREKNFHIFYYIYAGLHHQKKLSDFRLPEEKPPRYIADETGRVMHDITSKESYRRQFEAIQHCFRIIGFTDKEVHSVYRILAGILNIGNIEFAAISSQHQTDKSEVPNAEALQNAASVLCISPEELQEALTSHCVVTRGETIIRANTVDRAADVRDAMSKALYGRLFSWIVNRINTLLQPDENICSAGGGMNVGILDIFGFENFQRNSFEQLCINIANEQIQYYFNQHVFALEQMEYQNEGIDAVPVEYEDNRPLLDMFLQKPLGLLALLDEESRFPQATDQTLVDKFEDNLRCKYFWRPKGVELCFGIQHYAGKVLYDASGVLEKNRDTLPADVVVVLRTSENKLLQQLFSIPLTKTGNLAQTRARITVASSSLPPHFSAGKAKVDTLEVIRHPEETTNMKRQTVASYFRYSLMDLLSKMVVGQPHFVRCIKPNDDREALQFSRERVLAQLRSTGILETVSIRRQGYSHRILFEEFVKRYYYLAFTAHQTPLASKESCVAILEKSRLDHWVLGKTKVFLKYYHVEQLNLLLREVIGRVVVLQAYTKGWLGARRYKRVREKREKGAIAIQSAWRGYDARRKFKKISNRRNESAAHNQAGDTSNQSSGPHSPVAAGTRGSAEVQDCSEPGDHKVLRGSVHRRSHSQAESNNGRTQTSSNSPAVTEKNGSHSCDIQTRAREEHPRQEKQEWAIARGRICRAILGRHSQAQSSPKGCDIFAGHANKHSVSGTDLLSSRICHPAPDQQGLSLWGAPQKPGSENGLAQKHRTPRRRCQQPKMLSSPEDTMYYNQLNGTLEYQGSKRKPRKLGQIKVLDGEDEYYKSLSPVDCIPEENNSAHPSFFSSSSKGDSFAQH

SEQ ID NO: 277 - protein, 1323 aa, Homo sapiens
MLQSALLSTRKHLYGLFHYNPMMLGLESLPDPTDTWEIIETIGKGTYGKVYKVTNKRDGSLAAVKILDPVSDMDEEIEAEYNILQFLPNHPNVVKFYGMFYKADHCVGGQLWLVLELCNGGSVTELVKGLLRCGQRLDEAMISYILYGALLGLQHLHNNRIIHRDVKGNNILLTTEGGVKLVDFGVSAQLTSTRLRRNTSVGTPFWMAPEVIACEQQYDSSYDARCDVWSLGITAIELGDGDPPLFDMHPVKTLFKIPRNPPPTLLHPEKWCEEFNHFISQCLIKDFERRPSVTHLLDHPFIKGVHGKVLFLQKQLAKVLQDQKHQNPVAKTRHERMHTRRPYHVEDAEKYCLEDDLVNLEVLDEDTIIHQLQKRYADLLIYTYVGDILIALNPFQNLSIYSPQFSRLYHGVKRASNPPHIFASADAAYQCMVTLSKDQCIVISGESGSGKTESAHLIVQHLTFLGKANNQTLREKILQVNSLVEAFGNSCTAINDNSSRFGKYLEMMFTPTGVVMGARISEYLLEKSRVIKQAAREKNFHIFYYIYAGLHHQKKLSDFRLPEEKPPRYIADETGRVMHDITSKESYRRQFEAIQHCFRIIGFTDKEVHSVYRILAGILNIGNIEFAAISSQHQTDKSEVPNAEALQNAASVLCISPEELQEALTSHCVVTRGETIIRANTVDRAADVRDAMSKALYGRLFSWIVNRINTLLQPDENICSAGGGMNVGILDIFGFENFQRNSFEQLCINIANEQIQYYFNQHVFALEQMEYQNEGIDAVPVEYEDNRPLLDMFLQKPLGLLALLDEESRFPQATDQTLVDKFEDNLRCKYFWRPKGVELCFGIQHYAGKVLYDASGVLEKNRDTLPADVVVVLRTSENKLLQQLFSIPLTKTGNLAQTRARITVASSSLPPHFSAGKAKVDTLEVIRHPEETTNMKRQTVASYFRYSLMDLLSKMVVGQPHFVRCIKPNDDREALQFSRERVLAQLRSTGILETVSIRRQGYSHRILFEEFVKRYYYLAFTAHQTPLASKESCVAILEKSRLDHWVLGKTKVFLKYYHVEQLNLLLREVIGRVVVLQAYTKGWLGARRYKRVREKREKGAIAIQSGDTSNQSSGPHSPVAAGTRGSAEVQDCSEPGDHKVLRGSVHRRSHSQAESNNGRTQTSSNSPAVTEKNGHSQAQSSPKGCDIFAGHANKHSVSGTDLLSSRICHPAPDQQGLSLWGAPQKPGSENGLAQKHRTPRRRCQQPKMLSSPEDTMYYNQLNGTLEYQGSKRKPRKLGQIKVLDGEDEYYKSLSPVDCIPEENNSAHPSFFSSSSKGDSFAQH

SEQ ID NO: 278 - protein, 1155 aa, Homo sapiens
MRCLIKDFERRPSVTHLLDHPFIKGVHGKVLFLQKQLAKVLQDQKHQNPVAKTSNWQGAGYIDVFQPTRKNSQISFHADKSIYLKLWFFKLKQKKKKTEKLRHERMHTRRPYHVEDAEKYCLEDDLVNLEVLDEDTIIHQLQKRYADLLIYTYVGDILIALNPFQNLSIYSPQFSRLYHGVKRASNPPHIFASADAAYQCMVTLSKDQCIVISGESGSGKTESAHLIVQHLTFLGKANNQTLREKILQVNSLVEAFGNSCTAINDNSSRFGKYLEMMFTPTGVVMGARISEYLLEKSRVIKQAAREKNFHIFYYIYAGLHHQKKLSDFRLPEEKPPRYIADETGRVMHDITSKESYRRQFEAIQHCFRIIGFTDKEVHSVYRILAGILNIGNIEFAAISSQHQTDKSEVPNAEALQNAASVLCISPEELQEALTSHCVVTRGETIIRANTVDRAADVRDAMSKALYGRLFSWIVNRINTLLQPDENICSAGGGMNVGILDIFGFENFQRNSFEQLCINIANEQIQYYFNQHVFALEQMEYQNEGIDAVPVEYEDNRPLLDMFLQKPLGLLALLDEESRFPQATDQTLVDKFEDNLRCKYFWRPKGVELCFGIQHYAGKVLYDASGVLEKNRDTLPADVVVVLRTSENKLLQQLFSIPLTKTGNLAQTRARITVASSSLPPHFSAGKAKVDTLEVIRHPEETTNMKRQTVASYFRYSLMDLLSKMVVGQPHFVRCIKPNDDREALQFSRERVLAQLRSTGILETVSIRRQGYSHRILFEEFVKRYYYLAFTAHQTPLASKESCVAILEKSRLDHWVLGKTKVFLKYYHVEQLNLLLREVIGRVVVLQAYTKGWLGARRYKRVREKREKGAIAIQSAWRGYDARRKFKKISNRRNESAAHNQAGDTSNQSSGPHSPVAAGTRGSAEVQDCSEPGDHKVLRGSVHRRSHSQAESNNGRTQTSSNSPAVTEKNGSHSCDIQTRAREEHPRQEKQEWAIARGRICRAILGRHSQAQSSPKGCDIFAGHANKHSVSGTDLLSSRICHPAPDQQGLSLWGAPQKPGSENGLAQKHRTPRRRCQQPKMLSSPEDTMYYNQLNGTLEYQGSKRKPRKLGQIKVLDGEDEYYKSLSPVDCIPEENNSAHPSFFSSSSKGDSFAQH

SEQ ID NO: 279 - protein, 1050 aa, Homo sapiens
MHTRRPYHVEDAEKYCLEDDLVNLEVLDEDTIIHQLQKRYADLLIYTYVGDILIALNPFQNLSIYSPQFSRLYHGVKRASNPPHIFASADAAYQCMVTLSKDQCIVISGESGSGKTESAHLIVQHLTFLGKANNQTLREKILQVNSLVEAFGNSCTAINDNSSRFGKYLEMMFTPTGVVMGARISEYLLEKSRVIKQAAREKNFHIFYYIYAGLHHQKKLSDFRLPEEKPPRYIADETGRVMHDITSKESYRRQFEAIQHCFRIIGFTDKEVHSVYRILAGILNIGNIEFAAISSQHQTDKSEVPNAEALQNAASVLCISPEELQEALTSHCVVTRGETIIRANTVDRAADVRDAMSKALYGRLFSWIVNRINTLLQPDENICSAGGGMNVGILDIFGFENFQRNSFEQLCINIANEQIQYYFNQHVFALEQMEYQNEGIDAVPVEYEDNRPLLDMFLQKPLGLLALLDEESRFPQATDQTLVDKFEDNLRCKYFWRPKGVELCFGIQHYAGKVLYDASGVLEKNRDTLPADVVVVLRTSENKLLQQLFSIPLTKTGNLAQTRARITVASSSLPPHFSAGKAKVDTLEVIRHPEETTNMKRQTVASYFRYSLMDLLSKMVVGQPHFVRCIKPNDDREALQFSRERVLAQLRSTGILETVSIRRQGYSHRILFEEFVKRYYYLAFTAHQTPLASKESCVAILEKSRLDHWVLGKTKVFLKYYHVEQLNLLLREVIGRVVVLQAYTKGWLGARRYKRVREKREKGAIAIQSAWRGYDARRKFKKISNRRNESAAHNQAGDTSNQSSGPHSPVAAGTRGSAEVQDCSEPGDHKVLRGSVHRRSHSQAESNNGRTQTSSNSPAVTEKNGSHSCDIQTRAREEHPRQEKQEWAIARGRICRAILGRHSQAQSSPKGCDIFAGHANKHSVSGTDLLSSRICHPAPDQQGLSLWGAPQKPGSENGLAQKHRTPRRRCQQPKMLSSPEDTMYYNQLNGTLEYQGSKRKPRKLGQIKVLDGEDEYYKSLSPVDCIPEENNSAHPSFFSSSSKGDSFAQH

SEQ ID NO: 280 - protein, 1050 aa, Homo sapiens; identical to SEQ ID NO: 279

SEQ ID NO: 281 - protein, 1050 aa, Homo sapiens; identical to SEQ ID NO: 279

SEQ ID NO: 282 - protein, 1960 aa, Homo sapiens
MAQQAADKYLYVDKNFINNPLAQADWAAKKLVWVPSDKSGFEPASLKEEVGEEAIVELVENGKKVKVNKDDIQKMNPPKFSKVEDMAELTCLNEASVLHNLKERYYSGLIYTYSGLFCVVINPYKNLPIYSEEIVEMYKGKKRHEMPPHIYAITDTAYRSMMQDREDQSILCTGESGAGKTENTKKVIQYLAYVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEEQIILEDQNCKLAKEKKLLEDRIAEFTTNLTEEEEKSKSLAKLKNKHEAMITDLEERLRREEKQRQELEKTRRKLEGDSTDLSDQIAELQAQIAELKMQLAKKEEELQAALARVEEEAAQKNMALKKIRELESQISELQEDLESERASRNKAEKQKRDLGEELEALKTELEDTLDSTAAQQELRSKREQEVNILKKTLEEEAKTHEAQIQEMRQKHSQAVEELAEQLEQTKRVKANLEKAKQTLENERGELANEVKVLLQGKGDSEHKRKKVEAQLQELQVKFNEGERVRTELADKVTKLQVELDNVTGLLSQSDSKSSKLTKDFSALESQLQDTQELLQEENRQKLSLSTKLKQVEDEKNSFREQLEEEEEAKHNLEKQIATLHAQVADMKKKMEDSVGCLETAEEVKRKLQKDLEGLSQRHEEKVAAYDKLEKTKTRLQQELDDLLVDLDHQRQSACNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDERKQRSMAVAARKKLEMDLKDLEAHIDSANKNRDEAIKQLRKLQAQMKDCMRELDDTRASREEILAQAKENEKKLKSMEAEMIQLQEELAAAERAKRQAQQERDELADEIANSSGKGALALEEKRRLEARIAQLEEELEEEQGNTELINDRLKKANLQIDQINTDLNLERSHAQKNENARQQLERQNKELKVKLQEMEGTVKSKYKASITALEAKIAQLEEQLDNETKERQAACKQVRRTEKKLKDVLLQVDDERRNAEQYKDQADKASTRLKQLKRQLEEAEEEAQRANASRRKLQRELEDATETADAMNREVSSLKNKLRRGDLPFVVPRRMARKGAGDGSDEEVDGKADGAEAKPAE

SEQ ID NO: 283 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 284 - protein, 164 aa, Homo sapiens; identical to SEQ ID NO: 283

SEQ ID NO: 285 - protein, 164 aa, Homo sapiens; identical to SEQ ID NO: 283

SEQ ID NO: 286 - protein, 198 aa, Homo sapiens
MWGVFRRQTTHSSNPPLPGQQSCCNHRDFFCSGAMSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 287 - protein, 198 aa, Homo sapiens; identical to SEQ ID NO: 286

SEQ ID NO: 288 - protein, 177 aa, Homo sapiens
MVGDRRPLKTPGAMSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 289 - protein, 197 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTGADMCTEGLCKGPGFSESMVQGLTCLVLVQHAPDFYHSKRRLIFSKRKP

SEQ ID NO: 290 - protein, 167 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTVCLNKHTELVVVPLLGSLT

SEQ ID NO: 291 - protein, 164 aa, Homo sapiens; identical to SEQ ID NO: 283

SEQ ID NO: 292 - protein, 164 aa, Homo sapiens; identical to SEQ ID NO: 283

SEQ ID NO: 293 - protein, 118 aa, Homo sapiens
MSDEEARQSGGSSQAGVVTVSDVQELMRRKEEIEAQIKANYDVLESGLQVDDEIVEFGSVNTQNFQSLHNIGSVVQHSEGKPLNVTVIRRGEKHQLRLVPTRWAGKGLLGCNIIPLQR

SEQ ID NO: 294 - protein, 223 aa, Homo sapiens
MSDEEARQSGGSSQAGVVTVSDVQELMRRKEEIEAQIKANYDVLESQKGIGMNEPLVDCEGYPRSDVDLYQVRTARHNIICLQNDHKAVMKQVEEALHQLHARDKEKQARDMAEAHKEAMSRKLGQSESQGPPRAFAKVNSISPGSPASIAGLQVDDEIVEFGSVNTQNFQSLHNIGSVVQHSEGKPLNVTVIRRGEKHQLRLVPTRWAGKGLLGCNIIPLQR

SEQ ID NO: 295 - protein, 591 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 296 - protein, 605 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 297 - protein, 1081 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 298 - protein, 989 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 299 - protein, 900 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 300 - protein, 852 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 301 - protein, 740 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 302 - protein, 694 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 303 - protein, 561 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRGAALARGAAP

SEQ ID NO: 304 - protein, 511 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRGAALARGAAP

SEQ ID NO: 305 - protein, 479 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRGAALARGAAP

SEQ ID NO: 306 - protein, 557 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 307 - protein, 424 aa, Homo sapiens
MSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 308 - protein, 403 aa, Homo sapiens
MSAPSSTGPQYCPGLPSRKEATPEGRPETCLLPSSPQAQRPIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 309 - protein, 1129 aa, Homo sapiens
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 310 - protein, 996 aa, Homo sapiens
MSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 311 - protein, 996 aa, Homo sapiens; identical to SEQ ID NO: 310

SEQ ID NO: 312 - protein, 996 aa, Homo sapiens; identical to SEQ ID NO: 310

SEQ ID NO: 313 - protein, 996 aa, Homo sapiens; identical to SEQ ID NO: 310

SEQ ID NO: 314 - protein, 996 aa, Homo sapiens; identical to SEQ ID NO: 310

SEQ ID NO: 315 - protein, 1135 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 316 - protein, 1087 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 317 - protein, 1057 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRGAALARGAAP

SEQ ID NO: 318 - protein, 961 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQAGLKGAPPFLSPVLMPEPSPTLHTHTCTHTYTDTLHPSIYKPQHSNHSPAVNLDPGLPPLRRSAALLLFCGSTHVCSFLDWSSVLPRAPIEERSHSRRETRNLSPPFQPPGPAPDPGPGAKSGWGAVLGGRLASGRRAEQPRRAGRGRGLLAPGGGCTSCLHQLICPGGRKVPPSLCCQTPYPEPQES

SEQ ID NO: 319 - protein, 940 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 320 - protein, 888 aa, Homo sapiens
MSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 321 - protein, 858 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 322 - protein, 748 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 323 - protein, 700 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 324 - protein, 645 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 325 - protein, 611 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 326 - protein, 597 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 327 - protein, 563 aa, Homo sapiens
MRTLREAKTADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 328 - protein, 424 aa, Homo sapiens; identical to SEQ ID NO: 307

SEQ ID NO: 329 - protein, 1130 aa, Homo sapiens
MAGNKDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 330 - protein, 1127 aa, Homo sapiens
MADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKTSSSTVALSAPGLSSSAPSSYCSLPPSPPPMPSVFLPPTTIPSPRGQGHTPEFPCTEQSGRGLLPPVAPSWLDLAGLGVMPDTFNSRSPSVEGSLWAVGTESQGRDWRHLPTITSELSGAPRCHTVPCAGSTALQEPGEPQGPPASPSCPEEALAATWEQPWASEVFGPERMPPSGAARSFQEVTEPAVVAVDRQAIFPDTWTLTEEHGLQQERPRPEPGRLGSSSPASVTTEQLGAKMTERGSVARPTQGPETPRSPEGTTEAATQDGKEQPELPCAMAMGTPSTTERISTSGQIRSVIRRSWESGHAHPMSREPSPRRRLDPATLSRTPSQEQLIAELQGRLGIQPEAEEPAEAAGPSAQDWLTEGVIITVQPRGKRAGGQLVEKVVFPPGSPIPLRRTISVLASPSVPLLQHRTDAAASSSSPLPSLLASSPLGPSAYTCGSSGVQSAGEEPHDEGVQGPALPIPAPHTMRSVGCQTDEDPLFPPMQIQGLEQRADGERCWAAGWPRDGGRSSPGGQDEGGGSWPLEEVVLLVSISSSVQEGEKYPHPCAARHRTPSLRSPDQPPPCPQFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 331 - protein, 589 aa, Homo sapiens
MADALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 332 - protein, 355 aa, Homo sapiens
MSAPSSTGPQYCPGLPSRKEATPEGRPETCLLPSSPQAQRPIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 333 - protein, 1101 aa, Homo sapiens
MESLLLPVLLLLAILWTQAAALINLKYSVEEEQRAGTVIANVAKDAREAGFALDPRQASAFRVVSNSAPHLVDINPSSGLLVTKQKIDRDLLCRQSPKCIISLEVMSSSMEICVIKVEIKDLNDNAPSFPAAQIELEISEAASPGTRIPLDSAYDPDSGSFGVQTYELTPNELFGLEIKTRGDGSRFAELVVEKSLDRETQSHYSFRITALDGGDPPRLGTVGLSIKVTDSNDNNPVFSESTYAVSVPENSPPNTPVIRLNASDPDEGTNGQVVYSFYGYVNDRTRELFQIDPHSGLVTVTGALDYEEGHVYELDVQAKDLGPNSIPAHCKVTVSVLDTNDNPPVINLLSVNSELVEVSESAPPGYVIALVRVSDRDSGLNGRVQCRLLGNVPFRLQEYESFSTILVDGRLDREQHDQYNLTIQARDGGVPMLQSAKSFTVLITDENDNHPHFSKPYYQVIVQENNTPGAYLLSVSARDPDLGLNGSVSYQIVPSQVRDMPVFTYVSINPNSGDIYALRSFNHEQTKAFEFKVLAKDGGLPSLQSNATVRVIILDVNDNTPVITAPPLINGTAEVYIPRNSGIGYLVTVVKAEDYDEGENGRVTYDMTEGDRGFFEIDQVNGEVRTTRTFGESSKSSYELIVVAHDHGKTSLSASALVLIYLSPALDAQESMGSVNLSLIFIIALGSIAGILFVTMIFVAIKCKRDNKEIRTYNCRIAEYSYGHQKKSSKKKKISKNDIRLVPRDVEETDKMNVVSCSSLTSSLNYFDYHQQTLPLGCRRSESTFLNVENQNTRNTSANHIYHHSFNSQGPQQPDLIINGVPLPETENYSFDSNYVNSRAHLIKSSSTFKDLEGNSLKDSGHEESDQTDSEHDVQRSLYCDTAVNDVLNTSVTSMGSQMPDHDQNEGFHCREECRILGHSDRCWMPRNPMPIRSKSPEHVRNIIALSIEATAADVEAYDDCGPTKRTFATFGKDVSDHPAEERPTLKGKRTVDVTICSPKVNSVIREAGNGCEAISPVTSPLHLKSSLPTKPSVSYTIALAPPARDLEQYVNNVNNGPTRPSEAEPRGADSEKVMHEVSPILKEGRNKESPGVKRLKDIVL

SEQ ID NO: 334 - protein, 1148 aa, Homo sapiens
MESLLLPVLLLLAILWTQAAALINLKYSVEEEQRAGTVIANVAKDAREAGFALDPRQASAFRVVSNSAPHLVDINPSSGLLVTKQKIDRDLLCRQSPKCIISLEVMSSSMEICVIKVEIKDLNDNAPSFPAAQIELEISEAASPGTRIPLDSAYDPDSGSFGVQTYELTPNELFGLEIKTRGDGSRFAELVVEKSLDRETQSHYSFRITALDGGDPPRLGTVGLSIKVTDSNDNNPVFSESTYAVSVPENSPPNTPVIRLNASDPDEGTNGQVVYSFYGYVNDRTRELFQIDPHSGLVTVTGALDYEEGHVYELDVQAKDLGPNSIPAHCKVTVSVLDTNDNPPVINLLSVNSELVEVSESAPPGYVIALVRVSDRDSGLNGRVQCRLLGNVPFRLQEYESFSTILVDGRLDREQHDQYNLTIQARDGGVPMLQSAKSFTVLITDENDNHPHFSKPYYQVIVQENNTPGAYLLSVSARDPDLGLNGSVSYQIVPSQVRDMPVFTYVSINPNSGDIYALRSFNHEQTKAFEFKVLAKDGGLPSLQSNATVRVIILDVNDNTPVITAPPLINGTAEVYIPRNSGIGYLVTVVKAEDYDEGENGRVTYDMTEGDRGFFEIDQVNGEVRTTRTFGESSKSSYELIVVAHDHGKTSLSASALVLIYLSPALDAQESMGSVNLSLIFIIALGSIAGILFVTMIFVAIKCKRDNKEIRTYNCSNCLTITCLLGCFIKGQNSKCLHCISVSPISEEQDKKTEEKVSLRGKRIAEYSYGHQKKSSKKKKISKNDIRLVPRDVEETDKMNVVSCSSLTSSLNYFDYHQQTLPLGCRRSESTFLNVENQNTRNTSANHIYHHSFNSQGPQQPDLIINGVPLPETENYSFDSNYVNSRAHLIKSSSTFKDLEGNSLKDSGHEESDQTDSEHDVQRSLYCDTAVNDVLNTSVTSMGSQMPDHDQNEGFHCREECRILGHSDRCWMPRNPMPIRSKSPEHVRNIIALSIEATAADVEAYDDCGPTKRTFATFGKDVSDHPAEERPTLKGKRTVDVTICSPKVNSVIREAGNGCEAISPVTSPLHLKSSLPTKPSVSYTIALAPPARDLEQYVNNVNNGPTRPSEAEPRGADSEKVMHEVSPILKEGRNKESPGVKRLKDIVL

SEQ ID NO: 335 - protein, 1100 aa, Homo sapiens
MESLLLPVLLLLAILWTQAAALINLKYSVEEEQRAGTVIANVAKDAREAGFALDPRQASAFRVVSNSAPHLVDINPSSGLLVTKQKIDRDLLCRQSPKCIISLEVMSSSMEICVIKVEIKDLNDNAPSFPAAQIELEISEAASPGTRIPLDSAYDPDSGSFGVQTYELTPNELFGLEIKTRGDGSRFAELVVEKSLDRETQSHYSFRITALDGGDPPRLGTVGLSIKVTDSNDNNPVFSESTYAVSVPENSPPNTPVIRLNASDPDEGTNGQVVYSFYGYVNDRTRELFQIDPHSGLVTVTGALDYEEGHVYELDVQAKDLGPNSIPAHCKVTVSVLDTNDNPPVINLLSVNSELVEVSESAPPGYVIALVRVSDRDSGLNGRVQCRLLGNVPFRLQEYESFSTILVDGRLDREQHDQYNLTIQARDGGVPMLQSAKSFTVLITDENDNHPHFSKPYYQVIVQENNTPGAYLLSVSARDPDLGLNGSVSYQIVPSQVRDMPVFTYVSINPNSGDIYALRSFNHEQTKAFEFKVLAKDGGLPSLQSNATVRVIILDVNDNTPVITAPPLINGTAEVYIPRNSGIGYLVTVVKAEDYDEGENGRVTYDMTEGDRGFFEIDQVNGEVRTTRTFGESSKSSYELIVVAHDHGKTSLSASALVLIYLSPALDAQESMGSVNLSLIFIIALGSIAGILFVTMIFVAIKCKRDNKEIRTYNCRIAEYSYGHQKKSSKKKKISKNDIRLVPRDVEETDKMNVVSCSSLTSSLNYFDYHQQTLPLGCRRSESTFLNVENQNTRNTSANHIYHHSFNSQGPQQPDLIINGVPLPETENYSFDSNYVNSRAHLIKSSTFKDLEGNSLKDSGHEESDQTDSEHDVQRSLYCDTAVNDVLNTSVTSMGSQMPDHDQNEGFHCREECRILGHSDRCWMPRNPMPIRSKSPEHVRNIIALSIEATAADVEAYDDCGPTKRTFATFGKDVSDHPAEERPTLKGKRTVDVTICSPKVNSVIREAGNGCEAISPVTSPLHLKSSLPTKPSVSYTIALAPPARDLEQYVNNVNNGPTRPSEAEPRGADSEKVMHEVSPILKEGRNKESPGVKRLKDIVL

SEQ ID NO: 336 - protein, 1147 aa, Homo sapiens
MESLLLPVLLLLAILWTQAAALINLKYSVEEEQRAGTVIANVAKDAREAGFALDPRQASAFRVVSNSAPHLVDINPSSGLLVTKQKIDRDLLCRQSPKCIISLEVMSSSMEICVIKVEIKDLNDNAPSFPAAQIELEISEAASPGTRIPLDSAYDPDSGSFGVQTYELTPNELFGLEIKTRGDGSRFAELVVEKSLDRETQSHYSFRITALDGGDPPRLGTVGLSIKVTDSNDNNPVFSESTYAVSVPENSPPNTPVIRLNASDPDEGTNGQVVYSFYGYVNDRTRELFQIDPHSGLVTVTGALDYEEGHVYELDVQAKDLGPNSIPAHCKVTVSVLDTNDNPPVINLLSVNSELVEVSESAPPGYVIALVRVSDRDSGLNGRVQCRLLGNVPFRLQEYESFSTILVDGRLDREQHDQYNLTIQARDGGVPMLQSAKSFTVLITDENDNHPHFSKPYYQVIVQENNTPGAYLLSVSARDPDLGLNGSVSYQIVPSQVRDMPVFTYVSINPNSGDIYALRSFNHEQTKAFEFKVLAKDGGLPSLQSNATVRVIILDVNDNTPVITAPPLINGTAEVYIPRNSGIGYLVTVVKAEDYDEGENGRVTYDMTEGDRGFFEIDQVNGEVRTTRTFGESSKSSYELIVVAHDHGKTSLSASALVLIYLSPALDAQESMGSVNLSLIFIIALGSIAGILFVTMIFVAIKCKRDNKEIRTYNCSNCLTITCLLGCFIKGQNSKCLHCISVSPISEEQDKKTEEKVSLRGKRIAEYSYGHQKKSSKKKKISKNDIRLVPRDVEETDKMNVVSCSSLTSSLNYFDYHQQTLPLGCRRSESTFLNVENQNTRNTSANHIYHHSFNSQGPQQPDLIINGVPLPETENYSFDSNYVNSRAHLIKSSTFKDLEGNSLKDSGHEESDQTDSEHDVQRSLYCDTAVNDVLNTSVTSMGSQMPDHDQNEGFHCREECRILGHSDRCWMPRNPMPIRSKSPEHVRNIIALSIEATAADVEAYDDCGPTKRTFATFGKDVSDHPAEERPTLKGKRTVDVTICSPKVNSVIREAGNGCEAISPVTSPLHLKSSLPTKPSVSYTIALAPPARDLEQYVNNVNNGPTRPSEAEPRGADSEKVMHEVSPILKEGRNKESPGVKRLKDIVL

SEQ ID NO: 337 - protein, 1070 aa, Homo sapiens
MSPVRRWGSPCLFPLQLFSLCWVLSVAQSKTVRYSTFEEDAPGTVIGTLAEDLHMKVSGDTSFRLMKQFNSSLLRVREGDGQLTVGDAGLDRERLCGQAPQCVLAFDVVSFSQEQFRLVHVEVEVRDVNDHAPRFPRAQIPVEVSEGAAVGTRIPLEVPVDEDVGANGLQTVRLAEPHSPFRVELQTRADGAQCADLVLLQELDRESQAAYSLELVAQDGGRPPRSATAALSVRVLDANDHSPAFPQGAVAEVELAEDAPVGSLLLDLDAADPDEGPNGDVVFAFGARTPPEARRLFRLDPRSGRLTLAGPVDYERQDTYELDVRAQDRGPGPRAATCKVIVRIRDVNDNAPDIAITPLAAPGAPATSPFAAAAAAAALGGADASSPAGAGTPEAGATSLVPEGAARESLVALVSTSDRDSGANGQVRCALYGHEHFRLQPAYAGSYLVVTAASLDRERIAEYNLTLVAEDRGAPPLRTVRPYTVRVGDENDNAPLFTRPVYEVSVRENNPPGAYLATVAARDRDLGRNGQVTYRLLEAEVGRAGGAVSTYVSVDPATGAIYALRSFDYETLRQLDVRIQASDGGSPQLSSSALVQVRVLDQNDHAPVLVHPAPANGSLEVAVPGRTAKDTVVARVQARDADEGANGELAFELQQQEPREAFAIGRRTGEILLTGDLSQEPPGRVFRALLVISDGGRPPLTTTATVSFVVTAGGGRGPAAPASAGSPERSRPPGSRLGVSGSVLQWDTPLIVIIVLAGSCTLLLAAIIAIATTCNRRKKEVRKGGALREERPGAAGGGASAPGSPEEAARGAGPRPNMFDVLTFPGTGKAPFGSPAADAPPPAVAAAEVPGSEGGSATGESACHFEGQQRLRGAHAEPYGASPGFGKEPAPPVAVWKGHSFNTISGREAEKFSGKDSGKGDSDFNDSDSDISGDALKKDLINHMQSGLWACTAECKILGHSDRCWSPSCSGPNAHPSPHPPAQMSTFCKSTSLPRDPLRRDNYYQAQLPKTVGLQSVYEKVLHRDYDRTVTLLSPPRPGRLPDLQEIGVPLYQSPPGRYLSPKKGANENV

SEQ ID NO: 338 - protein, 973 aa, Homo sapiens
MSPVRRWGSPCLFPLQLFSLCWVLSVAQSKTVRYSTFEEDAPGTVIGTLAEDLHMKVSGDTSFRLMKQFNSSLLRVREGDGQLTVGDAGLDRERLCGQAPQCVLAFDVVSFSQEQFRLVHVEVEVRDVNDHAPRFPRAQIPVEVSEGAAVGTRIPLEVPVDEDVGANGLQTVRLAEPHSPFRVELQTRADGAQCADLVLLQELDRESQAAYSLELVAQDGGRPPRSATAALSVRVLDANDHSPAFPQGAVAEVELAEDAPVGSLLLDLDAADPDEGPNGDVVFAFGARTPPEARRLFRLDPRSGRLTLAGPVDYERQDTYELDVRAQDRGPGPRAATCKVIVRIRDVNDNAPDIAITPLAAPGAPATSPFAAAAAAAALGGADASSPAGAGTPEAGATSLVPEGAARESLVALVSTSDRDSGANGQVRCALYGHEHFRLQPAYAGSYLVVTAASLDRERIAEYNLTLVAEDRGAPPLRTVRPYTVRVGDENDNAPLFTRPVYEVSVRENNPPGAYLATVAARDRDLGRNGQVTYRLLEAEVGRAGGAVSTYVSVDPATGAIYALRSFDYETLRQLDVRIQASDGGSPQLSSSALVQVRVLDQNDHAPVLVHPAPANGSLEVAVPGRTAKDTVVARVQARDADEGANGELAFELQQQEPREAFAIGRRTGEILLTGDLSQEPPGRVFRALLVISDGGRPPLTTTATVSFVVTAGGGRGPAAPASAGSPERSRPPGSRLGVSGSVLQWDTPLIVIIVLAGSCTLLLAAIIAIATTCNRRKKEPYGASPGFGKEPAPPVAVWKGHSFNTISGREAEKFSGKDSGKGDSDFNDSDSDISGDALKKDLINHMQSGLWACTAECKILGHSDRCWSPSCSGPNAHPSPHPPAQMSTFCKSTSLPRDPLRRDNYYQAQLPKTVGLQSVYEKVLHRDYDRTVTLLSPPRPGRLPDLQEIGVPLYQSPPGRYLSPKKGANENV

SEQ ID NO: 339 - protein, 261 aa, Homo sapiens
MFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS

SEQ ID NO: 340 - protein, 261 aa, Homo sapiens; identical to SEQ ID NO: 339

SEQ ID NO: 341 - protein, 562 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 342 - protein, 473 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 343 - protein, 516 aa, Homo sapiens
MDAMKRGLCCVLLLCGAVFVSPSQEIHARFRRGARSYQGCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 344 - protein, 510 aa, Homo sapiens
MGRPTQWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKLSVAPQFLPRSLRVADLAANQVMEIFPLTFGEKPALRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQVRHRLVSHTVPRAPPSPCLPCHVPNILVSW

SEQ ID NO: 345 - protein, 421 aa, Homo sapiens
MGRPTQWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQVRHRLVSHTVPRAPPSPCLPCHVPNILVSW

SEQ ID NO: 346 - protein, 574 aa, Homo sapiens
MWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKLSVAPQFLPRSLRVADLAANQVMEIFPLTFGEKPALRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 347 - protein, 505 aa, Homo sapiens
MWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKLSVAPQFLPRSLRVADLAANQVMEIFPLTFGEKPALRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQVRHRLVSHTVPRAPPSPCLPCHVPNILVSW

SEQ ID NO: 348 - protein, 536 aa, Homo sapiens
MAESGLAMWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 349 - protein, 492 aa, Homo sapiens
MAESGLAMWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 350 - protein, 579 aa, Homo sapiens
MGRPTQWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKLSVAPQFLPRSLRVADLAANQVMEIFPLTFGEKPALRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 351 - protein, 563 aa, Homo sapiens
MAESGLAMWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEALSGPSVSAPVPPCRGSGCQPSDGDLPPHLWGEAGTQVRVPPQQPAEQRWPAPRRLPRLRGHRHPQPLQQPAQLPAAQPAALTRAAPPAGSPANNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 352 - protein, 551 aa, Homo sapiens
MAESGLAMWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEALSGPSVSAPVPPCRGSGCQPSDGDLPPHLWGEAGTQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 353 - protein, 548 aa, Homo sapiens
MAESGLAMWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKVRVPPQQPAEQRWPAPRRLPRLRGHRHPQPLQQPAQLPAAQPAALTRAAPPAGSPANNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 354 - protein, 499 aa, Homo sapiens
MAWTFECSRTTSPEPLSTSPCRTTSSRNSPTMSCPASVACEPSTSTTTSSPPKLSVAPQFLPRSLRVADLAANQVMEIFPLTFGEKPALRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 355 - protein, 484 aa, Homo sapiens
MAWTFECSRTTSPEPLSTSPCRTTSSRNSPTMSCPASVACEPSTSTTTSSPPKACLTRPSSPSPSCSTSAWLTTRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 356 - protein, 410 aa, Homo sapiens
MAWTFECSRTTSPEPLSTSPCRTTSSRNSPTMSCPASVACEPSTSTTTSSPPKNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 357 - protein, 415 aa, Homo sapiens
MAWTFECSRTTSPEPLSTSPCRTTSSRNSPTMSCPASVACEPSTSTTTSSPPKACLTRPSSPSPSCSTSAWLTTRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQVRHRLVSHTVPRAPPSPCLPCHVPNILVSW

SEQ ID NO: 358 - protein, 423 aa, Homo sapiens
MAESGLAMWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQVRHRLVSHTVPRAPPSPCLPCHVPNILVSW

SEQ ID NO: 359 - protein, 176 aa, Homo sapiens
MRKHLSWWWLATVCMLLFSHLSAVQTRGIKHRIKWNRKALPSTAQITEAQVAENRPGAFIKQGRKLDIDFGAEGNRYYEANYWQFPDGIHYNGCSEANVTKEAFVTGCINATQAANQGEFQKPDNKLHQQVLWRLVQELCSLKHCEFWLERGAGLRVTMHQPVLLCLLALIWLTVK

SEQ ID NO: 360 - protein, 490 aa, Homo sapiens
MTTIPRKGSSHLPGSLHTCKLKLQEDRRQQEKSVIAQPIFVFEKGEQTFKRPAEDTLYEAAEPECNGFPTKRVRSSSFTFHITDSQSQGVSTLSQKQMRCSSVTNLPTFPHSGPVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSDPSSWTHRQSVACS

SEQ ID NO: 361 - protein, 494 aa, Homo sapiens
MTTIPRKGSSHLPGSLHTCKLKLQEDRRQQEKSVIAQPIFVFEKGEQTFKRPAEDTLYEAAEPECNGFPTKRVRSSSFTFHITDSQSQGVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSETPHESHKKISLSTYSPISFFNSVLSHHGFEVTLIVTPKQLSD

SEQ ID NO: 362 - protein, 478 aa, Homo sapiens
MTTIPRKGSSHLPGSLHTCKLKLQEDRRQQEKSVIAQPIFVFEKGEQTFKRPAEDTLYEAAEPECNGFPTKRVRSSSFTFHITDSQSQGVSTLSQKQMRCSSVTNLPTFPHSGPVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSGK

SEQ ID NO: 363 - protein, 470 aa, Homo sapiens
MTTIPRKGSSHLPGSLHTCKLKLQEDRRQQEKSVIAQPIFVFEKGEQTFKRPAEDTLYEAAEPDSQSQGVSTLSQKQMRCSSVTNLPTFPHSGPVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSDPSSWTHRQSVACS

SEQ ID NO: 364 - protein, 458 aa, Homo sapiens
MTTIPRKGSSHLPGSLHTCKLKLQEDRRQQEKSVIAQPIFVFEKGEQTFKRPAEDTLYEAAEPDSQSQGVSTLSQKQMRCSSVTNLPTFPHSGPVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSGK

SEQ ID NO: 365 - protein, 453 aa, Homo sapiens
MTTIPRKGSSHLPGSLHTCKLKLQEDRRQQEKSVIAQPIFVFEKGEQTFKRPAEDTLYEAAEPECNGFPTKRVRSSSFTFHITDSQSQGVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSGK

SEQ ID NO: 366 - protein, 445 aa, Homo sapiens
MTTIPRKGSSHLPGSLHTCKLKLQEDRRQQEKSVIAQPIFVFEKGEQTFKRPAEDTLYEAAEPDSQSQGVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSDPSSWTHRQSVACS

SEQ ID NO: 367 - protein, 441 aa, Homo sapiens
MTTIPRKGSSHLPGSLHTCKLKLQEDRRQQEKSVIAQPIFVFEKGEQTFKRPAEDTLYEAAEPECNGFPTKRVRSSSFTFHITDSQSQGGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSDPSSWTHRQSVACS

SEQ ID NO: 368 - protein, 433 aa, Homo sapiens
MTTIPRKGSSHLPGSLHTCKLKLQEDRRQQEKSVIAQPIFVFEKGEQTFKRPAEDTLYEAAEPDSQSQGVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSGK

SEQ ID NO: 369 - protein, 465 aa, Homo sapiens
MTTIPRKGSSHLPGSLHTCKLKLQEDRRQQEKSVIAQPIFVFEKGEQTFKRPAEDTLYEAAEPECNGFPTKRVRSSSFTFHITDSQSQGVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSDPSSWTHRQSVACS

SEQ ID NO: 370 - protein, 519 aa, Homo sapiens
MTTIPRKGSSHLPGSLHTCKLKLQEDRRQQEKSVIAQPIFVFEKGEQTFKRPAEDTLYEAAEPECNGFPTKRVRSSSFTFHITDSQSQGVSTLSQKQMRCSSVTNLPTFPHSGPVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSETPHESHKKISLSTYSPISFFNSVLSHHGFEVTLIVTPKQLSD

SEQ ID NO: 371 - protein, 499 aa, Homo sapiens
MTTIPRKGSSHLPGSLHTCKLKLQEDRRQQEKSVIAQPIFVFEKGEQTFKRPAEDTLYEAAEPDSQSQGVSTLSQKQMRCSSVTNLPTFPHSGPVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSETPHESHKKISLSTYSPISFFNSVLSHHGFEVTLIVTPKQLSD

SEQ ID NO: 372 - protein, 474 aa, Homo sapiens
MTTIPRKGSSHLPGSLHTCKLKLQEDRRQQEKSVIAQPIFVFEKGEQTFKRPAEDTLYEAAEPDSQSQGVRKNNVFMTSALVQSSVDIKSAEQGPVKHSKHVIRPAILQLPQARSCAKVRKTFGHKALESCKTKEKTNNKISEGNSYLLSENLSRARISVQLSTNQDFLGATSVGCQPNEDKCSFKSCSSNFVFGENMVERVLGTQKLTQPQLENDSYAKEKPFKSIPKFPVNFLSSRTDSIKNTSLIESAAAFSSQPSRKCLLEKIDVITGEETEHNVLKINCKLFIFNKTTQSWIERGRGTLRLNDTASTDCGTLQSRLIMRNQGSLRLILNSKLWAQMKIQRANHKNVRITATDLEDYSIKIFLIQASAQDTAYLYAAIHHRLVALQSFNKQRDVNQAESLSETAQQLNCESCDENEDDFIQVTKNGSETPHESHKKISLSTYSPISFFNSVLSHHGFEVTLIVTPKQLSD

SEQ ID NO: 373 - protein, 238 aa, Homo sapiens
MRALLALCLLLGWLRWGPAGAQQSGEYCHGWVDVQGNYHEGFQCPEDFDTLDATICCGSCALRYCCAAADARLEQGGCTNDRRELEHPGITAQPVYVPFLIVGSIFIAFIILGSVVAIYCCTCLRPKEPSQQPIRFSLRSYQTETLPMILTSTSPRAPSRQSSTATSSSSTGGSIRRFSFARAEPGCLVPSPPPPYTTSHSIHLAQPSGFLVSPQYFAYPLQQEPPLPGKSCPDFSSS

SEQ ID NO: 374 - protein, 597 aa, Homo sapiens
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 375 - protein, 592 aa, Homo sapiens
MTSRRWFHPNITGVEAENLLLTRGVDGSFLARPSKSNPGDFTLSVRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 376 - protein, 593 aa, Homo sapiens [sequence omitted]

SEQ ID NO: 377 - protein, 460 aa, Homo sapiens
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCR

SEQ ID NO: 378 - protein, 596 aa, Homo sapiens
MTSRRWFHPNITGVEAENLLLTRGVDGSFLARPSKSNPGDFTLSVRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 379 - protein, 644 aa, Homo sapiens
MDLQQSTTITSLEKWCLDESLSGCRRHYSVKKKLKLIRVLGLFMGLVAISTVSFSISAFSETDTQSTGEASVVSGPRVAQGYHQRTLLDLNDKILDYTPQPPLSKEGESENSTDHAQGDYPKDIFSLEERRKGAIILHVIGMIYMFIALAIVCDEFFVPSLTVITEKLGISDDVAGATFMAAGGSAPELFTSLIGVFIAHSNVGIGTIVGSAVFNILFVIGMCALFSREILNLTWWPLFRDVSFYIVDLIMLIIFFLDNVIMWWESLLLLTAYFCYVVFMKFNVQVEKWVKQMINRNKVVKVTAPEAQAKPSAARDKDEPTLPAKPRLQRGGSSASLHNSLMRNSIFQLMIHTLDPLAEGRFREKASILHKIAKKKCHVDENERQNGAANHVEKIELPNSTSTDVEMTPSSDASEPVQNGNLSHNIEGAEAQTADEEEDQPLSLAWPSETRKQVTFLIVFPIVFPLWITLPDVRKPSSRKFFPITFFGSITWIAVFSYLMVWWAHQVGETIGISEEIMGLTILAAGTSIPDLITSVIVARKGLGDMAVSSSVGSNIFDITVGLPLPWLLYTVIHRFQPVAVSSNGLFCAIVLLFIMLLFVILSIALCKWRMNKILGFIMFGLYFVFLVVSVLLEDRILTCPVSI

SEQ ID NO: 380 - protein, 661 aa, Homo sapiens
MDLQQSTTITSLEKWCLDESLSGCRRHYSVKKKLKLIRVLGLFMGLVAISTVSFSISAFSETDTQSTGEASVVSGPRVAQGYHQRTLLDLNDKILDYTPQPPLSKEGESENSTDHAQGDYPKDIFSLEERRKGAIILHVIGMIYMFIALAIVCDEFFVPSLTVITEKLGISDDVAGATFMAAGGSAPELFTSLIGVFIAHSNVGIGTIVGSAVFNILFVIGMCALFSREILNLTWWPLFRDVSFYIVDLIMLIIFFLDNVIMWWESLLLLTAYFCYVVFMKFNVQVEKWVKQMINRNKVVKVTAPEAQAKPSAARDKDEPTLPAKPRLQRGGSSASLHNSLMRNSIFQLMIHTLDPLAEELGSYGKLKYYDTMTEEGRFREKASILHKIAKKKCHVDENERQNGAANHVEKIELPNSTSTDVEMTPSSDASEPVQNGNLSHNIEGAEAQTADEEEDQPLSLAWPSETRKQVTFLIVFPIVFPLWITLPDVRKPSSRKFFPITFFGSITWIAVFSYLMVWWAHQVGETIGISEEIMGLTILAAGTSIPDLITSVIVARKGLGDMAVSSSVGSNIFDITVGLPLPWLLYTVIHRFQPVAVSSNGLFCAIVLLFIMLLFVILSIALCKWRMNKILGFIMFGLYFVFLVVSVLLEDRILTCPVSI

SEQ ID NO: 381 - protein, 644 aa, Homo sapiens; identical to SEQ ID NO: 379

SEQ ID NO: 382 - protein, 661 aa, Homo sapiens; identical to SEQ ID NO: 380

SEQ ID NO: 383 - protein, 661 aa, Homo sapiens; identical to SEQ ID NO: 380

SEQ ID NO: 384 - protein, 552 aa, Homo sapiens
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 385 - protein, 300 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 386 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 387 - protein, 287 aa, Homo sapiens
MRIAVICFCLLGITCAIPVKQADSGSSEEKQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 388 - protein, 273 aa, Homo sapiens
MRIAVICFCLLGITCAIPVKQADSGSSEEKQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 389 - protein, 327 aa, Homo sapiens
MGIVPRSLDKKAHRVQFQLNRIKAKIELPWGSLQLDCLMKTLKELYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 390 - protein, 354 aa, Homo sapiens
MQLQFRSWMLAALTLLVVFLIFADISEIEEEIGAEVVINGSSSPAVVDRSNESIKHNIQPASSKWRHNQTLSLRIRKQILKFLDAEKDISVLKGTLKPGDIIHYIFDRDSTMNVSQNLYELLPRTSPLKNKHFGTCAIVGNSGVLLNSGCGQEIDAHSFVIRCNLAPVQEYARDVGLKTDLVTMNPSVIQRAFEDLVNATWREKLLQRLHSLNGSILWIPAFMARGGKERVEWVNELILKHHVNVRTAYPSLRLLHAVRGYWLTNKVHIKRPTTGLLMYTLATRFCKQIYLYGFWPFPLDQNQNPVKYHYYDSLKYGYTSQASPHTMPLEFKALKSLHEQGALKLTVGQCDGAT

SEQ ID NO: 391 - protein, 375 aa, Homo sapiens
MQLQFRSWMLAALTLLVVFLIFADISEIEEEIGNSGGRGTIRSAVNSLHSKSNRAEVVINGSSSPAVVDRSNESIKHNIQPASSKWRHNQTLSLRIRKQILKFLDAEKDISVLKGTLKPGDIIHYIFDRDSTMNVSQNLYELLPRTSPLKNKHFGTCAIVGNSGVLLNSGCGQEIDAHSFVIRCNLAPVQEYARDVGLKTDLVTMNPSVIQRAFEDLVNATWREKLLQRLHSLNGSILWIPAFMARGGKERVEWVNELILKHHVNVRTAYPSLRLLHAVRGYWLTNKVHIKRPTTGLLMYTLATRFCKQIYLYGFWPFPLDQNQNPVKYHYYDSLKYGYTSQASPHTMPLEFKALKSLHEQGALKLTVGQCDGAT

SEQ ID NO: 392 - protein, 396 aa, Homo sapiens
MFSLPFLLFTVHPSEGVRKLSLNILRVKVELGTIPKTVFSFATSFYFYSVVSKRNSGGRGTIRSAVNSLHSKSNRAEVVINGSSSPAVVDRSNESIKHNIQPASSKWRHNQTLSLRIRKQILKFLDAEKDISVLKGTLKPGDIIHYIFDRDSTMNVSQNLYELLPRTSPLKNKHFGTCAIVGNSGVLLNSGCGQEIDAHSFVIRCNLAPVQEYARDVGLKTDLVTMNPSVIQRAFEDLVNATWREKLLQRLHSLNGSILWIPAFMARGGKERVEWVNELILKHHVNVRTAYPSLRLLHAVRGYWLTNKVHIKRPTTGLLMYTLATRFCKQIYLYGFWPFPLDQNQNPVKYHYYDSLKYGYTSQASPHTMPLEFKALKSLHEQGALKLTVGQCDGAT

SEQ ID NO: 393 - protein, 1114 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPNGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 394 - protein, 787 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKLWRPETQRQGASGAVLPLLLPASLAQASLGFWRWWLHGPDLWSASHHFLQCLSLRKLPLPLFDKNTCGCI

SEQ ID NO: 395 - protein, 334 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVVGTHAGAAGVIGHGQLRRWPISAGINIPWLGSASCSM

SEQ ID NO: 396 - protein, 1095 aa, Homo sapiens
MRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 397 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 398 - protein, 415 aa, Homo sapiens
MEQIWLLLLLTIRVLPGSAQFNGYNCDANLHSRFPAERDISVYCGVQAITMKINFCTVLFSGYSETDLALNGRHGDSHCRGFINNNTFPAVVIFIINLSTLEGCGNNLVVSTIPGVSAYGNATSVQVGNISGYIDTPDPPTIISYLPGLLYKFSCSYPLEYLVNNTQLASSSAAISVRENNGTFVSTLNLLLYNDSTYNQQLIIPSIGLPLKTKVFAAVQATNLDGRWNVLMDYCYTTPSGNPNDDIRYDLFLSCDKDPQTTVIENGRSQRGRFSFEVFRFVKHKNQKMSTVFLHCVTKLCRADDCPFLMPICSHRERRDAGRRTTWSPQSSSGSAVLSAGPIITRSDETPTNNSQLGSPSMPPFQLNAITSALISGMVILGVTSFSLLLCSLALLHRKGPTSLVLNGIRNPVFD

SEQ ID NO: 399 - protein, 431 aa, Homo sapiens
MMLRFSMCRGNDEGFAMEQIWLLLLLTIRVLPGSAQFNGYNCDANLHSRFPAERDISVYCGVQAITMKINFCTVLFSGYSETDLALNGRHGDSHCRGFINNNTFPAVVIFIINLSTLEGCGNNLVVSTIPGVSAYGNATSVQVGNISGYIDTPDPPTIISYLPGLLYKFSCSYPLEYLVNNTQLASSSAAISVRENNGTFVSTLNLLLYNDSTYNQQLIIPSIGLPLKTKVFAAVQATNLDGRWNVLMDYCYTTPSGNPNDDIRYDLFLSCDKDPQTTVIENGRSQRGRFSFEVFRFVKHKNQKMSTVFLHCVTKLCRADDCPFLMPICSHRERRDAGRRTTWSPQSSSGSAVLSAGPIITRSDETPTNNSQLGSPSMPPFQLNAITSALISGMVILGVTSFSLLLCSLALLHRKGPTSLVLNGIRNPVFD

SEQ ID NO: 400 - protein, 415 aa, Homo sapiens; identical to SEQ ID NO: 398

SEQ ID NO: 401 - protein, 415 aa, Homo sapiens; identical to SEQ ID NO: 398

(19) United States
(12) Patent Application Publication
(10) Pub. No.: US 2024/0336981 A1
(43) Pub. Date: Oct. 10, 2024

(54) PANELS AND METHODS FOR DIAGNOSING AND TREATING LUNG CANCER

(71) Applicants: The Broad Institute, Inc., Cambridge, MA (US); The General Hospital Corporation, Boston, MA (US)

(72) Inventors: Yifat GEFFEN, Cambridge, MA (US); Whijae ROH, Cambridge, MA (US); Gad GETZ, Boston, MA (US)

(73) Assignees: The Broad Institute, Inc., Cambridge, MA (US); The General Hospital Corporation, Boston, MA (US)

(21) Appl. No.: 18/750,868

(22) Filed: Jun. 21, 2024

Related U.S. Application Data
(63) Continuation of application No. PCT/US2022/082233, filed on Dec. 22, 2022.
(60) Provisional application No. 63/293,349, filed on Dec. 23, 2021.
(60) Provisional application No. 63/373,535, filed on Aug. 25, 2022.

(51) Int. Cl. C12Q 1/6886; C12Q 1/6851

(52) U.S. Cl. CPC C12Q 1/6886; C12Q 1/6851; C12Q 2600/106; C12Q 2600/158

(57) ABSTRACT

The disclosure provides molecular classifiers for use in the characterization and diagnosis of lung cancer and methods of selecting and treating subjects with appropriate personalized cancer treatments, including but not limited to CDK4/6 inhibitors, c-Met inhibitors, PD-1/PD-L1 inhibitors and combinations thereof.

### CROSS-REFERENCE TO RELATED APPLICATIONS

This application is a continuation under 35 U.S.C. § 111(a) of International Application No. PCT/US2022/082233, filed Dec. 22, 2022, which claims priority to and benefit of U.S. Provisional Applications No. 63/373,535, filed Aug. 25, 2022, and 63/293,349, filed Dec. 23, 2021, the entire contents of each of which is hereby incorporated herein by reference in its entirety.

### STATEMENT REGARDING FEDERALLY SPONSORED RESEARCH OR DEVELOPMENT

This invention was made with government support under Grant No. CA210999 awarded by the National Institutes of Health. The government has certain rights in the invention.

### SEQUENCE LISTING

This application contains a Sequence Listing which has been filed electronically in compliance with ST.26 format and is hereby incorporated by reference in its entirety. The Sequence Listing, created on Jun. 27, 2024, is named 167741-042003US_SL.xml and is 654.508 bytes in size.

### BACKGROUND OF THE INVENTION

Lung cancer is the most prevalent cause of death from cancer worldwide. One of the reasons that lung cancer is so prevalent, is that the symptoms of the disease are rarely detected before the cancer has become invasive. While many cancer immunotherapies and checkpoint inhibitors have been developed to treat lung cancer, some subsets of cancer patients do not respond to these treatments. This is largely due to differential genomic, transcriptomic, and proteomic profiles between individual patient tumors. Therefore, there is a great unmet need for personalized cancer diagnostics that identify subsets of patients that will be responsive to cancer therapies.

### SUMMARY OF THE INVENTION

As provided herein, the present disclosure features panels and methods that can be used to characterize, diagnose, and administer personalized cancer treatment to a subject with lung cancer. The methods provided herein are based, at least in part, on the discovery of new lung adenocarcinoma subtypes with unique gene and polypeptide expression signatures and corresponding subtype-specific therapeutic targets.

In one aspect, the disclosure features a panel for characterizing a lung cancer in a biological sample of a subject. The panel contains two or more polypeptide or polynucleotide markers, or fragments thereof, selected from those listed in Table 1, or fragments thereof.

In another aspect, the disclosure features a panel for characterizing an S2 subtype lung adenocarcinoma in a biological sample of a subject. The panel contains two or more polypeptide or polynucleotide markers, or fragments thereof, selected from ARAF_pS299, BAP1C4, BIM, C7orf10, CAPNS2, CASPASE7CLEAVEDD198, CILP2, CLAUDIN7, CMET_pY1235, COL8A2, CXorf64, CYCLINE1, CYP26A1, DBC1, DIO1, EGFR_pY1068, ENPP3, FIBRONECTIN, FNDC1, GPR88, IBSP, INPP4B, ISM1, ITGA11, LIPK, LRRTM1, MAPK_pT202Y204, MATN3, MFAP5, MMP11, MYO3B, MYOSINIIA_pS1943, P21, P27, P63, PAXILLIN, PCDH19, PCDH8, PCNA, PLAT, PODNL1, PRND, RANBP3L, SHISA3, SHP2_pY542, SLC24A2, SMAD4, SPP1, ST8SIA2, THBS2, and ZPLD1.

In another aspect, the disclosure features a panel for characterizing an S2 subtype lung adenocarcinoma in a biological sample of a subject. The panel contains two or more polypeptide or polynucleotide markers, or fragments thereof, selected from SLC24A2, C7orf10, MFAP5, GPR88, MATN3, FNDC1, RANBP3L, CILP2, PCDH19, SPP1, CAPNS2, ZPLD1, ENPP3, PRND, PLAT, PODNL1, LIPK, SHISA3, CXorf64, DIO1, PCDH8, DBC1, and MYO3B.

In another aspect, the disclosure features a panel for characterizing an S2 subtype lung adenocarcinoma in a biological sample of a subject. The panel contains two or more polypeptide or polynucleotide markers, or fragments thereof, selected from SLC24A2, COL8A2, C7orf10, CYP26A1, and MMP11.

In another aspect, the disclosure features a panel for characterizing an S2 subtype lung adenocarcinoma in a biological sample of a subject. The panel contains two or more polypeptide or polynucleotide markers, or fragments thereof, selected from BIM, CMET_pY1235, CASPASE7CLEAVEDD198, CLAUDIN7, CYCLINE1, EGFR_pY1068, FIBRONECTIN, INPP4B, MAPK_pT202Y204, P27, PAXILLIN, PCNA, SMAD4, ARAF_pS299, BAP1C4, MYOSINIIA_pS1943, P21, SHP2_pY542, and P63.

In another aspect, the disclosure features a panel for characterizing an S2 subtype lung adenocarcinoma in a biological sample of a subject. The panel contains two or more polypeptide or polynucleotide markers, or fragments thereof, selected from BIM, CLAUDIN7, EGFR_pY1068, P27, and ARAF_pS299.

In another aspect, the disclosure features a panel for characterizing an S3 subtype lung adenocarcinoma in a biological sample of a subject. The panel contains two or more polypeptide or polynucleotide markers, or fragments thereof, selected from AFAP1L2, AIM2, ANNEXINVII, ARNTL2, BATF3, BRD4, C10orf55, C12orf70, C15orf48, CATSPER1, CD20, CD274, CD70, CD8A, CDA, CHK1_pS345, CSF2, DCBLD2, DJ1, ERALPHA, FBXO32, GATA3, GATA6, GBP1, GPR84, GZMB, IFNG, JAK2, KCNK12, LCK, MET, MIG6, MYBL1, NKG7, P63, P70S6K1, PDCD1, PDL1, PEA15, PI3KP110ALPHA, PKCDELTA_pS664, S100A2, SYNAPTOPHYSIN, TBX21, TGM4, TIGAR, TMEM156, and TTF1.

In another aspect, the disclosure features a panel for characterizing an S3 subtype lung adenocarcinoma in a biological sample of a subject. The panel contains two or more polypeptide or polynucleotide markers, or fragments thereof, selected from AFAP1L2, ARNTL2, BATF3, C15orf48, CATSPER1, CD274, CD70, CD8A, CDA, CSF2, DCBLD2, FBXO32, GPR84, GZMB, KCNK12, MET, MYBL1, NKG7, S100A2, TBX21, TGM4, and TMEM156.

In another aspect, the disclosure features a panel for characterizing an S3 subtype lung adenocarcinoma in a biological sample of a subject. The panel contains two or more polypeptide or polynucleotide markers, or fragments thereof, selected from of AIM2, CD274, DCBLD2, FBXO32, and MYBL1.

In another aspect, the disclosure features a panel for characterizing an S3 subtype lung adenocarcinoma in a biological sample of a subject. The panel contains two or more polypeptide or polynucleotide markers, or fragments thereof, selected from ANNEXINVII, BRD4, CD20, CHK1pS345, DJ1, ERALPHA, GATA3, GATA6, JAK2, LCK, MIG6, P63, PDCD1, PDL1, PEA15, PI3KP110ALPHA, PKCDELTA_pS664, SYNAPTOPHYSIN, TIGAR, and TTF1.

In another aspect, the disclosure features a panel for characterizing an S3 subtype lung adenocarcinoma in a biological sample of a subject. The panel contains two or more polypeptide or polynucleotide markers, or fragments thereof, selected from GATA6, JAK2, MIG6, P70S6K1, and PDL1.

In another aspect, the disclosure features a panel for characterizing an lung adenocarcinoma S4 subtype in a biological sample of a subject. The panel contains two or more polypeptide or polynucleotide markers, or fragments thereof, selected from ACETYLATUBULINLYS40, AKR1C2, AKR1C4, AMPKALPHA, ANNEXIN1, BIM, C12orf39, C12orf56, C20orf70, CALB1, CALCA, CASPASE7CLEAVEDD198, CAVEOLIN1, CPS1, CSAG2, CYCLINB1, DUSP4, F2, F7, FOXM1, GLDC, GNG4, HEPACAM2, HOXD11, HOXD13, IGF2BP1, INSL4, JNK2, KCNU1, KLK14, LOC100190940, LOC441177, MIG6, MLLT11, MSH6, MTOR_pS2448, NAPSINA, NCADHERIN, NRF2, P38MAPK, P90RSK, PAH, PCSK1, PEA15, PKCALPHA_pS657, PKCPANBETAII_pS660, POPDC3, SLC38A8, SYNAPTOPHYSIN, TFRC, TIGAR, TTF1, UCHL1, UGT3A1, VEGFR2, WDR72, YAP_pS127, and ZMAT4.

In another aspect, the disclosure features a panel for characterizing an lung adenocarcinoma S4 subtype in a biological sample of a subject. The panel contains two or more polypeptide or polynucleotide markers, or fragments thereof, selected from AKR1C2, AKR1C4, C12orf39, C12orf56, C20orf70, CALB1, CPS1, CSAG2, F2, F7, GNG4, HEPACAM2, HOXD11, HOXD13, IGF2BP1, INSL4, KCNU1, KLK14, LOC100190940, MLLT11, PCSK1, POPDC3, SLC38A8, UCHL1, UGT3A1, WDR72, and ZMAT4.

In another aspect, the disclosure features a panel for characterizing an lung adenocarcinoma S4 subtype in a biological sample of a subject. The panel contains two or more polypeptide or polynucleotide markers, or fragments thereof, selected from AKR1C4, CALCA, HOXD13, MLLT11, and PAH.

In another aspect, the disclosure features a panel for characterizing an lung adenocarcinoma S4 subtype in a biological sample of a subject. The panel contains two or more polypeptide or polynucleotide markers, or fragments thereof, selected from ACETYLATUBULINLYS40, AMPKALPHA, ANNEXIN1, BIM, CASPASE7CLEAVEDD198, CAVEOLIN1, CYCLINB1, JNK2, MIG6, MSH6, MTOR_pS2448, NAPSINA, NCADHERIN, NRF2, P38MAPK, P90RSK, PEA15, PKCALPHA_pS657, SYNAPTOPHYSIN, TFRC, TIGAR, TTF1, VEGFR2, and YAP_pS127.

In another aspect, the disclosure features a panel for characterizing an lung adenocarcinoma S4 subtype in a biological sample of a subject. The panel contains two or more polypeptide or polynucleotide markers, or fragments thereof, selected from BIM, CAVEOLIN1, FOXM1, NRF2, and PKCPANBETAII_pS660.

In another aspect, the disclosure features a method of treating a subject selected as having a subtype 2 lung adenocarcinoma. The method involves administering to the selected subject an EGFR inhibitor and/or a TGF-beta inhibitor. The subject is selected by detecting in a biological sample obtained from the subject the level of one or more markers selected from ARAF_pS299, BAP1C4, BIM, C7orf10, CAPNS2, CASPASE7CLEAVEDD198, CILP2, CLAUDIN7, CMET_pY1235, COL8A2, CXorf64, CYCLINE1, CYP26A1, DBC1, DIO1, EGFR_pY1068, ENPP3, FIBRONECTIN, FNDC1, GPR88, IBSP, INPP4B, ISM1, ITGA11, LIPK, LRRTM1, MAPK_pT202Y204, MATN3, MFAP5, MMP11, MYO3B, MYOSINIIA_pS1943, P21, P27, P63, PAXILLIN, PCDH19, PCDH8, PCNA, PLAT, PODNL1, PRND, RANBP3L, SHISA3, SHP2_pY542, SLC24A2, SMAD4, SPP1, ST8SIA2, THBS2, and ZPLD1.

In another aspect, the disclosure features a method of treating a subject selected as having a subtype 3 lung adenocarcinoma. The method involves administering to the selected subject a c-Met inhibitor, a CDK4/6 inhibitor, a PD-1, and/or a PD-L1 checkpoint inhibitor. The subject is selected by detecting in a biological sample obtained from the subject the level of one or more markers selected from AFAP1L2, AIM2, ANNEXINVII, ARNTL2, BATF3, BRD4, C10orf55, C12orf70, C15orf48, CATSPER1, CD20, CD274, CD70, CD8A, CDA, CHK1_pS345, CSF2, DCBLD2, DJ1, ERALPHA, FBXO32, GATA3, GATA6, GBP1, GPR84, GZMB, IFNG, JAK2, KCNK12, LCK, MET, MIG6, MYBL1, NKG7, P63, P70S6K1, PDCD1, PDL1, PEA15, PI3KP110ALPHA, PKCDELTA_pS664, S100A2, SYNAPTOPHYSIN, TBX21, TGM4, TIGAR, TMEM156, and TTF1.

In another aspect, the disclosure features a method of treating a subject selected as having a subtype 4 lung adenocarcinoma. The method involves administering to the selected subject a c-Met inhibitor and/or a CDK4/6 inhibitor. The subject is selected by detecting in a biological sample obtained from the subject the level of one or more markers selected from ACETYLATUBULINLYS40, AKR1C2, AKR1C4, AMPKALPHA, ANNEXIN1, BIM, C12orf39, C12orf56, C20orf70, CALB1, CALCA, CASPASE7CLEAVEDD198, CAVEOLIN1, CPS1, CSAG2, CYCLINB1, DUSP4, F2, F7, FOXM1, GLDC, GNG4, HEPACAM2, HOXD11, HOXD13, IGF2BP1, INSL4, JNK2, KCNU1, KLK14, LOC100190940, LOC441177, MIG6, MLLT11, MSH6, MTOR_pS2448, NAPSINA, NCADHERIN, NRF2, P38MAPK, P90RSK, PAH, PCSK1, PEA15, PKCALPHA_pS657, PKCPANBETAII_pS660, POPDC3, SLC38A8, SYNAPTOPHYSIN, TFRC, TIGAR, TTF1, UCHL1, UGT3A1, VEGFR2, WDR72, YAP_pS127, and ZMAT4.

In another aspect, the disclosure features a method for selecting a subject for inclusion in or exclusion from a clinical trial of an agent for the treatment of an S2 lung adenocarcinoma. The method involves detecting in a biological sample obtained from the subject the level of one or more markers selected from ARAF_pS299, BAP1C4, BIM, C7orf10, CAPNS2, CASPASE7CLEAVEDD198, CILP2, CLAUDIN7, CMET_pY1235, COL8A2, CXorf64, CYCLINE1, CYP26A1, DBC1, DIO1, EGFR_pY1068, ENPP3, FIBRONECTIN, FNDC1, GPR88, IBSP, INPP4B, ISM1, ITGA11, LIPK, LRRTM1, MAPK_pT202Y204, MATN3, MFAP5, MMP11, MYO3B, MYOSINIIA_pS1943, P21, P27, P63, PAXILLIN, PCDH19, PCDH8, PCNA, PLAT, PODNL1, PRND, RANBP3L, SHISA3, SHP2_pY542, SLC24A2, SMAD4, SPP1, ST8SIA2, THBS2, and ZPLD1 relative to a corresponding reference level. Detecting an alteration in the level relative to the reference level indicates that the subject is a good candidate for the clinical trial.

In another aspect, the disclosure features a method for selecting a subject for inclusion in or exclusion from a clinical trial of an agent for the treatment of an S3 lung adenocarcinoma. The method involves detecting in a biological sample obtained from the subject the level of one or more markers selected from AFAP1L2, AIM2, ANNEXINVII, ARNTL2, BATF3, BRD4, C10orf55, C12orf70, C15orf48, CATSPER1, CD20, CD274, CD70, CD8A, CDA, CHK1pS345, CSF2, DCBLD2, DJ1, ERALPHA, FBXO32, GATA3, GATA6, GBP1, GPR84, GZMB, IFNG, JAK2, KCNK12, LCK, MET, MIG6, MYBL1, NKG7, P63, P70S6K1, PDCD1, PDL1, PEA15, PI3KP110ALPHA, PKCDELTA_pS664, S100A2, SYNAPTOPHYSIN, TBX21, TGM4, TIGAR, TMEM156, and TTF1 relative to a corresponding reference level. Detecting an alteration in the level relative to the reference level indicates that the subject is a good candidate for the clinical trial.

In another aspect, the disclosure features a method for selecting a subject for inclusion in or exclusion from a clinical trial of an agent for the treatment of S4 lung adenocarcinoma. The method involves detecting in a biological sample obtained from the subject the level of one or more markers selected from ACETYLATUBULINLYS40, AKR1C2, AKR1C4, AMPKALPHA, ANNEXIN1, BIM, C12orf39, C12orf56, C20orf70, CALB1, CALCA, CASPASE7CLEAVEDD198, CAVEOLIN1, CPS1, CSAG2, CYCLINB1, DUSP4, F2, F7, FOXM1, GLDC, GNG4, HEPACAM2, HOXD11, HOXD13, IGF2BP1, INSL4, JNK2, KCNU1, KLK14, LOC100190940, LOC441177, MIG6, MLLT11, MSH6, MTOR_pS2448, NAPSINA, NCADHERIN, NRF2, P38MAPK, P90RSK, PAH, PCSK1, PEA15, PKCALPHA_pS657, PKCPANBETAII_pS660, POPDC3, SLC38A8, SYNAPTOPHYSIN, TFRC, TIGAR, TTF1, UCHL1, UGT3A1, VEGFR2, WDR72, YAP_pS127, and ZMAT4 relative to a corresponding reference level. Detecting an alteration in the level relative to the reference level indicates that the subject is a good candidate for the clinical trial.

In another aspect, the disclosure features a method of treating a subject selected as having a subtype 2 lung adenocarcinoma. The method involves administering to the selected subject an EGFR inhibitor and/or a TGF-beta inhibitor. The subject is selected by detecting in a biological sample obtained from the subject the level of each of the following markers: SLC24A2, C7orf10, MFAP5, GPR88, MATN3, FNDC1, RANBP3L, CILP2, PCDH19, SPP1, CAPNS2, ZPLD1, ENPP3, PRND, PLAT, PODNL1, LIPK, SHISA3, CXorf64, DIO1, PCDH8, DBC1, and MYO3B. The markers are polynucleotides.

In another aspect, the disclosure features a method of treating a subject selected as having a subtype 2 lung adenocarcinoma. The method involves administering to the selected subject an EGFR inhibitor and/or a TGF-beta inhibitor. The subject is selected by detecting in a biological sample obtained from the subject the level of each of the following markers: SLC24A2, COL8A2, C7orf10, CYP26A1, and MMP11. The markers are polynucleotides.

In another aspect, the disclosure features a method of treating a subject selected as having a subtype 2 lung adenocarcinoma. The method involves administering to the selected subject an EGFR inhibitor and/or a TGF-beta inhibitor. The subject is selected by detecting in a biological sample obtained from the subject the level of each of the following markers: BIM, CMET_pY1235, CASPASE7CLEAVEDD198, CLAUDIN7, CYCLINE1, EGFR_pY1068, FIBRONECTIN, INPP4B, MAPK_pT202Y204, P27, PAXILLIN, PCNA, SMAD4, ARAF_pS299, BAP1C4, MYOSINIIA_pS1943, P21, SHP2_pY542, and P63. The markers are polypeptides.

In another aspect, the disclosure features a method of treating a subject selected as having a subtype 2 lung adenocarcinoma. The method involves administering to the selected subject an EGFR inhibitor and/or a TGF-beta inhibitor. The subject is selected by detecting in a biological sample obtained from the subject the level of each of the following markers: BIM, CLAUDIN7, EGFR_pY1068, P27, and ARAF_pS299. The markers are polypeptides.

In another aspect, the disclosure features a method of treating a subject selected as having a subtype 3 lung adenocarcinoma. The method involves administering to the selected subject a c-Met inhibitor, a CDK4/6 inhibitor, a PD-1, and/or a PD-L1 checkpoint inhibitor. The subject is selected by detecting in a biological sample obtained from the subject the level of each of the following markers: AFAP1L2, ARNTL2, BATF3, C15orf48, CATSPER1, CD274, CD70, CD8A, CDA, CSF2, DCBLD2, FBXO32, GPR84, GZMB, KCNK12, MET, MYBL1, NKG7, S100A2, TBX21, TGM4, and TMEM156. The markers are polynucleotides.

In another aspect, the disclosure features a method of treating a subject selected as having a subtype 3 lung adenocarcinoma. The method involves administering to the selected subject a c-Met inhibitor, a CDK4/6 inhibitor, a PD-1, and/or a PD-L1 checkpoint inhibitor. The subject is selected by detecting in a biological sample obtained from the subject the level of each of the following markers: AIM2, CD274, DCBLD2, FBXO32, and MYBL1. The markers are polynucleotides.

In another aspect, the disclosure features a method of treating a subject selected as having a subtype 3 lung adenocarcinoma. The method involves administering to the selected subject a c-Met inhibitor, a CDK4/6 inhibitor, a PD-1, and/or a PD-L1 checkpoint inhibitor. The subject is selected by detecting in a biological sample obtained from the subject the level of each of the following markers: ANNEXINVII, BRD4, CD20, CHK1_pS345, DJ1, ERALPHA, GATA3, GATA6, JAK2, LCK, MIG6, P63, PDCD1, PDL1, PEA15, PI3KP110ALPHA, PKCDELTA_pS664, SYNAPTOPHYSIN, TIGAR, and TTF1. The markers are polypeptides.

In another aspect, the disclosure features a method of treating a subject selected as having a subtype 3 lung adenocarcinoma. The method involves administering to the selected subject a c-Met inhibitor, a CDK4/6 inhibitor, a PD-1, and/or a PD-L1 checkpoint inhibitor. The subject is selected by detecting in a biological sample obtained from the subject the level of each of the following markers: GATA6, JAK2, MIG6, P70S6K1, and PDL1. The markers are polypeptides.

In another aspect, the disclosure features a method of treating a subject selected as having a subtype 4 lung adenocarcinoma. The method involves administering to the selected subject a c-Met inhibitor, a CDK4/6 inhibitor, a PD-1, and/or a PD-L1 checkpoint inhibitor. The subject is selected by detecting in a biological sample obtained from the subject the level of each of the following markers: AKR1C2, AKR1C4, C12orf39, C12orf56, C20orf70, CALB1, CPS1, CSAG2, F2, F7, GNG4, HEPACAM2, HOXD11, HOXD13, IGF2BP1, INSL4, KCNU1, KLK14, LOC100190940, MLLT11, PCSK1, POPDC3, SLC38A8, UCHL1, UGT3A1, WDR72, and ZMAT4. The detected levels are polynucleotides.

In another aspect, the disclosure features a method of treating a subject selected as having a subtype 4 lung adenocarcinoma. The method involves administering to the selected subject a c-Met inhibitor, a CDK4/6 inhibitor, a PD-1, and/or a PD-L1 checkpoint inhibitor. The subject is selected by detecting in a biological sample obtained from the subject the level of each of the following markers: AKR1C4, CALCA, HOXD13, MLLT11, and PAH. The markers are polynucleotides.

In another aspect, the disclosure features a method of treating a subject selected as having a subtype 4 lung adenocarcinoma. The method involves administering to the selected subject a c-Met inhibitor, a CDK4/6 inhibitor, a PD-1, and/or a PD-L1 checkpoint inhibitor. The subject is selected by detecting in a biological sample obtained from the subject the level of each of the following markers: ACETYLATUBULINLYS40, AMPKALPHA, ANNEXIN1, BIM, CASPASE7CLEAVEDD198, CAVEOLIN1, CYCLINB1, JNK2, MIG6, MSH6, MTOR_pS2448, NAPSINA, NCADHERIN, NRF2, P38MAPK, P90RSK, PEA15, PKCALPHA_pS657, SYNAPTOPHYSIN, TFRC, TIGAR, TTF1, VEGFR2, and YAP_pS127. The markers are polypeptides.

In another aspect, the disclosure features a method of treating a subject selected as having a subtype S4 lung adenocarcinoma. The method involves administering to the selected subject a c-Met inhibitor, a CDK4/6 inhibitor, a PD-1, and/or a PD-L1 checkpoint inhibitor. The subject is selected by detecting in a biological sample obtained from the subject the level of each of the following markers: BIM, CAVEOLIN1, FOXM1, NRF2, and PKCPANBETAII_pS660. The detected levels are polypeptides.

In any of the above aspects, or embodiments thereof, the markers are polynucleotides. In any of the above aspects, or embodiments thereof, the markers are polypeptides.

In any of the above aspects, or embodiments thereof, the markers are polypeptides used to characterize an S4 lung adenocarcinoma subtype.

In any of the above aspects, or embodiments thereof, the markers are bound to a capture molecule. In embodiments, the capture molecule is bound to a substrate selected from at least one of chips, beads, microfluidic platforms, and membranes.

In any of the above aspects, or embodiments thereof, the panel contains at least 5 or 10 markers. In any of the above aspects, or embodiments thereof, each capture molecule binds a marker of any one of the above aspects. In any of the above aspects, or embodiments thereof, the capture molecules contains an antibody or antigen binding fragment thereof. In any of the above aspects, or embodiments thereof, the capture molecules contains a polynucleotide.

In any of the above aspects, or embodiments thereof, the one or more markers are selected from SLC24A2, C7orf10, MFAP5, GPR88, MATN3, FNDC1, RANBP3L, CILP2, PCDH19, SPP1, CAPNS2, ZPLD1, ENPP3, PRND, PLAT, PODNL1, LIPK, SHISA3, CXorf64, DIO1, PCDH8, DBC1, and MYO3B. In any of the above aspects, or embodiments thereof, the one or more markers are selected from SLC24A2, COL8A2, C7orf10, CYP26A1, and MMP11.

In any of the above aspects, or embodiments thereof, the one or more markers are selected from BIM, CMET_pY1235, CASPASE7CLEAVEDD198, CLAUDIN7, CYCLINE1, EGFR_pY1068, FIBRONECTIN, INPP4B, MAPK_pT202Y204, P27, PAXILLIN, PCNA, SMAD4, ARAF_pS299, BAP1C4, MYOSINIIA_pS1943, P21, SHP2_pY542, and P63. In any of the above aspects, or embodiments thereof, the one or more markers are selected from BIM, CLAUDIN7, EGFR_pY1068, P27, and ARAF_pS299.

In any of the above aspects, or embodiments thereof, the method involves detecting the level of at least 5 of the markers. In any of the above aspects, or embodiments thereof, the method involves detecting the level of at least 10 of the markers.

In any of the above aspects, or embodiments thereof, the method further involves using the detected level of the one or more markers to classify the selected subject as having a subtype 2 lung adenocarcinoma. The classification has an accuracy of at least about 80%.

In any of the above aspects, or embodiments thereof, the subject is treated with an EGFR inhibitor and a TGF-beta inhibitor. In any of the above aspects, or embodiments thereof, the EGFR inhibitor is selected from one or more of Erlotinib, Osimertinib, Neratinib, Gefitinib, Cetuximab, Panitumumab, Dacomitinib, Lapatinib, Necitumumab, Mobocertinib, Vandetanib, and pharmaceutically acceptable salts thereof. In any of the above aspects, or embodiments thereof, the TGF-beta inhibitor is selected from one or more of Galunisertib, Vactosertib, Trabedersen, ISTH0036, Fresolimumab, Disitertide Belagenpumatucel-L, Gemogenovatucel-T, and pharmaceutically acceptable salts thereof.

In any of the above aspects, or embodiments thereof, the one or more markers are selected from AFAP1L2, ARNTL2, BATF3, C15orf48, CATSPER1, CD274, CD70, CD8A, CDA, CSF2, DCBLD2, FBXO32, GPR84, GZMB, KCNK12, MET, MYBL1, NKG7, S100A2, TBX21, TGM4, and TMEM156. In any of the above aspects, or embodiments thereof, the one or more markers are selected from AIM2, CD274, DCBLD2, FBXO32, and MYBL1. In any of the above aspects, or embodiments thereof, the one or more markers are selected from ANNEXINVII, BRD4, CD20, CHK1_pS345, DJ1, ERALPHA, GATA3, GATA6, JAK2, LCK, MIG6, P63, PDCD1, PDL1, PEA15, PI3KP110ALPHA, PKCDELTA_pS664, SYNAPTOPHYSIN, TIGAR, and TTF1. In any of the above aspects, or embodiments thereof, the one or more markers are selected from GATA6, JAK2, MIG6, P70S6K1, and PDL1.

In any of the above aspects, or embodiments thereof, the method further involves using the detected level of the one or more markers to classify the selected subject as having a subtype 3 lung adenocarcinoma. The classification has an accuracy of at least about 80%.

In any of the above aspects, or embodiments thereof, the subject is treated with a c-Met inhibitor and a CDK4/6 inhibitor. In any of the above aspects, or embodiments thereof, the subject is treated with a c-Met inhibitor and a PD-1 or a PD-L1 checkpoint inhibitor. In any of the above aspects, or embodiments thereof, the subject is treated with a CDK4/6 inhibitor and a PD-1 or a PD-L1 checkpoint inhibitor. In any of the above aspects, or embodiments thereof, the subject is treated with a c-Met inhibitor, a CDK4/6 inhibitor, and a PD-1 or PD-L1 checkpoint inhibitor.

In any of the above aspects, or embodiments thereof, the c-Met inhibitor is selected from one or more of AMG337, BMS 777607, cabozantinib, capmatinib, crizotinib, emibetuzumab, ficlatuzumab, foretinib, glesatinib, onartuzumab, rilotumumab, tepotinib, tivantinib, volitinib, and pharmaceutically acceptable salts thereof. In any of the above aspects, or embodiments thereof, the CDK4/6 inhibitor is selected from one or more of abemaciclib, AT7519, CINK4, flavopiridol, palbociclib, ribociclib, and pharmaceutically acceptable salts thereof. In any of the above aspects, or embodiments thereof, the PD-1/PD-L1 checkpoint inhibitor is selected from one or more of atezolizumab, avelumab, BMS-936559, MDX-1105, cemiplimab, durvalumab, nivolumab, pembrolizumab, and pharmaceutically acceptable salts thereof.

In any of the above aspects, or embodiments thereof, the one or more markers are selected from AKR1C2, AKR1C4, C12orf39, C12orf56, C20orf70, CALB1, CPS1, CSAG2, F2, F7, GNG4, HEPACAM2, HOXD11, HOXD13, IGF2BP1, INSL4, KCNU1, KLK14, LOC100190940, MLLT11, PCSK1, POPDC3, SLC38A8, UCHL1, UGT3A1, WDR72, and ZMAT4. In any of the above aspects, or embodiments thereof, one or more markers are selected from AKR1C4, CALCA, HOXD13, MLLT11, and PAH. In any of the above aspects, or embodiments thereof, one or more markers are selected from ACETYLATUBULINLYS40, AMPKALPHA, ANNEXIN1, BIM, CASPASE7CLEAVEDD198, CAVEOLIN1, CYCLINB1, JNK2, MIG6, MSH6, MTOR_pS2448, NAPSINA, NCADHERIN, NRF2, P38MAPK, P90RSK, PEA15, PKCALPHA_pS657, SYNAPTOPHYSIN, TFRC, TIGAR, TTF1, VEGFR2, and YAP_pS127. In any of the above aspects, or embodiments thereof, the one or more markers are selected from BIM, CAVEOLIN1, FOXM1, NRF2, and PKCPANBETAII_pS660.

In any of the above aspects, or embodiments thereof, the method further involves using the detected level of the one or more markers to classify the selected subject as having a subtype 4 lung adenocarcinoma. The classification has an accuracy of at least about 80%.

In any of the above aspects, or embodiments thereof, the subject is treated with a c-Met inhibitor and a CDK4/6 inhibitor. In any of the above aspects, or embodiments thereof, the method further involves selecting the subject for treatment with a PD-1 or PD-L1 checkpoint inhibitor. In any of the above aspects, or embodiments thereof, the method further involves administering to the subject at least one additional chemotherapeutic agent.

In any of the above aspects, or embodiments thereof, the method further involves using the detected alteration in the level of the one or more markers to classify the selected subject as having a subtype 2 lung adenocarcinoma. The classification has an accuracy of at least about 80%.

In any of the above aspects, or embodiments thereof, the agent contains an EGFR inhibitor. In any of the above aspects, or embodiments thereof, the agent contains a TGF-beta inhibitor. In any of the above aspects, or embodiments thereof, the agent contains a TGF-beta inhibitor and an EGFR inhibitor.

In any of the above aspects, or embodiments thereof, the subject is identified as having an S2 lung cancer subtype.

In any of the above aspects, or embodiments thereof, the EGFR inhibitor is selected from one or more of Erlotinib, Osimertinib, Neratinib, Gefitinib, Cetuximab, Panitumumab, Dacomitinib, Lapatinib, Necitumumab, Mobocertinib, Vandetanib, and pharmaceutically acceptable salts thereof. In any of the above aspects, or embodiments thereof, the TGF-beta inhibitor is selected from one or more of Galunisertib, Vactosertib, Trabedersen, ISTH0036, Fresolimumab, Disitertide, Belagenpumatucel-L, Gemogenovatucel-T, and pharmaceutically acceptable salts thereof.

In any of the above aspects, or embodiments thereof, the method further involves using the detected alteration in the level of the one or more markers to classify the selected subject as having a subtype 3 lung adenocarcinoma. The classification has an accuracy of at least about 80%.

In any of the above aspects, or embodiments thereof, the agent contains a c-Met inhibitor, a PD-1/PD-L1 checkpoint inhibitor, and/or a CDK4/6 inhibitor. In any of the above aspects, or embodiments thereof, the agent contains a c-Met inhibitor and a CDK4/6 inhibitor. In any of the above aspects, or embodiments thereof, the agent contains a c-Met inhibitor and a PD-1/PD-L1 checkpoint inhibitor. In any of the above aspects, or embodiments thereof, the agent contains a CDK4/6 inhibitor and a PD-1/PD-L1 checkpoint inhibitor. In any of the above aspects, or embodiments thereof, the agent contains a c-Met inhibitor, a CDK4/6 inhibitor, and a PD-1/PD-L1 checkpoint inhibitor.

In any of the above aspects, or embodiments thereof, the subject is identified as having an S3 lung cancer subtype.

In any of the above aspects, or embodiments thereof, the agent contains a c-Met inhibitor selected from one or more of AMG337, BMS 777607, cabozantinib, capmatinib, crizotinib, emibetuzumab, ficlatuzumab, foretinib, glesatinib, onartuzumab, rilotumumab, tepotinib, tivantinib, volitinib, and pharmaceutically acceptable salts thereof. In any of the above aspects, or embodiments thereof, the agent contains a CDK4/6 inhibitor selected from one or more of abemaciclib, AT7519, CINK4, flavopiridol, palbociclib, ribociclib, and pharmaceutically acceptable salts thereof. In any of the above aspects, or embodiments thereof, the agent contains a PD-1/PD-L1 checkpoint inhibitor selected from one or more of atezolizumab, avelumab, BMS-936559, MDX-1105, cemiplimab, durvalumab, nivolumab, pembrolizumab, and pharmaceutically acceptable salts thereof.

In any of the above aspects, or embodiments thereof, the method further involves using the detected alteration in the level of the one or more markers to classify the selected subject as having a subtype 4 lung adenocarcinoma. The classification has an accuracy of at least about 80%.

In any of the above aspects, or embodiments thereof, the agent contains a c-Met inhibitor, a PD-1/PD-L1 checkpoint inhibitor, and/or a CDK4/6 inhibitor. In any of the above aspects, or embodiments thereof, the agent contains a c-Met inhibitor and a CDK4/6 inhibitor. In any of the above aspects, or embodiments thereof, the agent contains a PD-1/PD-L1 checkpoint inhibitor.

In any of the above aspects, or embodiments thereof, the subject is identified as having an S4 lung cancer subtype.

In any of the above aspects, or embodiments thereof, the detected levels are used to classify the subtype 2 lung adenocarcinoma with an accuracy of at least 80%. In any of the above aspects, or embodiments thereof, the detected levels are used to classify the subtype 3 lung adenocarcinoma with an accuracy of at least 80%. In any of the above aspects, or embodiments thereof, the detected levels are used to classify the subtype 4 lung adenocarcinoma with an accuracy of at least 80%.

The disclosure provides panels and methods that can be used to characterize lung cancer subtypes and identify appropriate cancer therapies. Compositions, methods, assays, and articles defined by the disclosure were isolated or otherwise manufactured in connection with the examples provided below. Other features and advantages of the disclosure will be apparent from the detailed description, and from the claims.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (3^{rd} edition. 2006); The Cambridge Dictionary of Science and Technology (Walker ed., 1990); The Glossary of Genetics, 5th Ed., R. Rieger et al. (eds.), Springer Verlag (1991); and Hale & Marham, The Harper Collins Dictionary of Biology (1991), The Biology of Cancer (2^{nd} edition, Weinberg et al., 2013), and Cancer: Principles and Practice of Oncology Primer of Molecular Biology in Cancer (3^{rd} edition, LWW, 2020). As used herein, the following terms have the meanings ascribed to them below, unless specified otherwise.

By “agent” is meant any small molecule chemical compound, antibody, nucleic acid molecule, polypeptide, or fragments of any of the aforementioned agents. In some embodiments, the agent provided herein is a chemotherapeutic agent. In some embodiments, the agent provided herein is a CDK4/6 inhibitor, a c-Met inhibitor, an EGFR inhibitor, a PD-1/PD-L1 checkpoint inhibitor, and/or a TGF-beta inhibitor.

By “ameliorate” is meant decrease, suppress, attenuate, diminish, arrest, or stabilize the development or progression of a disease.

By “alteration” or “modulation” is meant a change (increase or decrease) in the expression levels, structure, or activity of a polynucleotide or polypeptide as detected by standard art known methods, such as those provided herein. As used herein, an alteration includes a 10% change in expression levels, a 25% change, a 40% change, or even a 50% or greater change in expression levels.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “biological sample” is meant any tissue, cell, fluid, or other material derived from an organism. Non-limiting examples of biological samples include a bodily fluid (such as blood, blood serum, plasma, saliva, urine, ascites, cyst fluid, and the like); a homogenized tissue sample (e.g., a tissue sample obtained by biopsy); and a cell isolated from a patient sample.

By “capture molecule” or “capture reagent” is meant a reagent that specifically binds a nucleic acid molecule or polypeptide to label, select, or isolate the nucleic acid molecule or polypeptide. Non-limiting examples of capture molecules include polynucleotide probes, antibodies, and fragments thereof.

By “decrease” is meant to alter negatively. A decrease may be by about or at least about 0.5%, 1%, 5%, 10%, 25%, 30%, 50%, 75%, or even by 100%.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “cyclin-dependent kinase 4 (CDK4) polypeptide” is meant a protein or a fragment thereof having cyclin-dependent kinase activity and having at least about 85% or greater amino acid sequence identity to NCBI Reference Sequence: NP_000066.1. An exemplary human CDK4 amino acid sequence is provided below:

>NP_000066.1 cyclin-dependent kinase 4
[Homo sapiens]
(SEQ ID NO. 1)
MATSRYEPVAEIGVGAYGTVYKARDPHSGHFVALKSVRVPNGGGGGGGLP
ISTVREVALLRRLEAFEHPNVVRLMDVCATSRTDREIKVTLVFEHVDQDL
RTYLDKAPPPGLPAETIKDLMRQFLRGLDFLHANCIVHRDLKPENILVTS
GGTVKLADFGLARIYSYQMALTPVVVTLWYRAPEVLLQSTYATPVDMWSV
GCIFAEMFRRKPLFCGNSEADQLGKIFDLIGLPPEDDWPRDVSLPRGAFP
PRGPRPVQSVVPEMEESGAQLLLEMLTFNPHKRISAFRALQHSYLHKDEG
NPE

By “CDK4 polynucleotide” is meant a nucleic acid molecule or fragment thereof encoding a CDK4 polypeptide. The sequence of an exemplary CDK4 polynucleotide is provided at NCBI Reference Sequence: NM_000075.4, which is reproduced below:

(SEQ ID NO. 2)
ATGGCTACCTCTCGATATGAGCCAGTGGCTGAAATTGGTGTCGGTGCCTA
TGGGACAGTGTACAAGGCCCGTGATCCCCACAGTGGCCACTTTGTGGCCC
TCAAGAGTGTGAGAGTCCCCAATGGAGGAGGAGGTGGAGGAGGCCTTCCC
ATCAGCACAGTTCGTGAGGTGGCTTTACTGAGGCGACTGGAGGCTTTTGA
GCATCCCAATGTTGTCCGGCTGATGGACGTCTGTGCCACATCCCGAACTG
ACCGGGAGATCAAGGTAACCCTGGTGTTTGAGCATGTAGACCAGGACCTA
AGGACATATCTGGACAAGGCACCCCCACCAGGCTTGCCAGCCGAAACGAT
CAAGGATCTGATGCGCCAGTTTCTAAGAGGCCTAGATTTCCTTCATGCCA
ATTGCATCGTTCACCGAGATCTGAAGCCAGAGAACATTCTGGTGACAAGT
GGTGGAACAGTCAAGCTGGCTGACTTTGGCCTGGCCAGAATCTACAGCTA
CCAGATGGCACTTACACCCGTGGTTGTTACACTCTGGTACCGAGCTCCCG
AAGTTCTTCTGCAGTCCACATATGCAACACCTGTGGACATGTGGAGTGTT
GGCTGTATCTTTGCAGAGATGTTTCGTCGAAAGCCTCTCTTCTGTGGAAA
CTCTGAAGCCGACCAGTTGGGCAAAATCTTTGACCTGATTGGGCTGCCTC
CAGAGGATGACTGGCCTCGAGATGTATCCCTGCCCCGTGGAGCCTTTCCC
CCCAGAGGGCCCCGCCCAGTGCAGTCGGTGGTACCTGAGATGGAGGAGTC
GGGAGCACAGCTGCTGCTGGAAATGCTGACTTTTAACCCACACAAGCGAA
TCTCTGCCTTTCGAGCTCTGCAGCACTCTTATCTACATAAGGATGAAGGT
AATCCGGAGT

By “cyclin-dependent kinase 6 (CDK6) polypeptide” is meant a polypeptide or a fragment thereof having cyclin-dependent kinase activity and having at least about 85% or greater amino acid sequence identity to NCBI Gene ID: 1021; or NCBI Reference Sequence: NP_001138778.1. An exemplary human CDK6 amino acid sequence is provided below:

>NP_001138778.1 cyclin-dependent kinase 6
[Homo sapiens]
(SEQ ID NO. 3)
MEKDGLCRADQQYECVAEIGEGAYGKVFKARDLKNGGRFVALKRVRVQTG
EEGMPLSTIREVAVLRHLETFEHPNVVRLFDVCTVSRTDRETKLTLVFEH
VDQDLTTYLDKVPEPGVPTETIKDMMFQLLRGLDFLHSHRVVHRDLKPQN
ILVTSSGQIKLADFGLARIYSFQMALTSVVVTLWYRAPEVLLQSSYATPV
DLWSVGCIFAEMFRRKPLFRGSSDVDQLGKILDVIGLPGEEDWPRDVALP
RQAFHSKSAQPIEKFVTDIDELGKDLLLKCLTFNPAKRISAYSALSHPYF
QDLERCKENLDSHLPPSQNTSELNTA

By “CDK6 polynucleotide” is meant a nucleic acid molecule encoding a CDK6 polypeptide. Exemplary CDK6 polynucleotide sequences are provided at NCBI Reference No. NM_001259.8 and NM_001145306. An exemplary CDK6 nucleic acid sequence is reproduced below:

NM_001259.8 Homo sapiens cyclin dependent kinase 6 (CDK6), transcript variant 1, mRNA
(SEQ ID NO. 4)
ACTGCGTCCCGCGCCGCTCGCTCATCCCCGAGGGGCCCCTGCAACCTCTCCGCGCGAAGACGGCTTCAGC
CCTGCAGGGAAAGAAAAGTGCAATGATTCTGGACTGAGACGCGCTTGGGCAGAGGCTATGTAATCGTGTC
TGTGTTGAGGACTTCGCTTCGAGGAGGGAAGAGGAGGGATCGGCTCGCTCCTCCGGCGGCGGCGGCGGCG
GCGACTCTGCAGGCGGAGTTTCGCGGCGGCGGCACCAGGGTTACGCCAGCCCCGCGGGGAGGTCTCTCCA
TCCAGCTTCTGCAGCGGCGAAAGCCCCAGCGCCCGAGCGCCTGAGCCGGCGGGGAGCAAGTAAAGCTAGA
CCGATCTCCGGGGAGCCCCGGAGTAGGCGAGCGGCGGCCGCCAGCTAGTTGAGCGCACCCCCCGCCCGCC
CCAGCGGCGCCGCGGCGGGCGGCGTCCAGGCGGCATGGAGAAGGACGGCCTGTGCCGCGCTGACCAGCAG
TACGAATGCGTGGCGGAGATCGGGGAGGGCGCCTATGGGAAGGTGTTCAAGGCCCGCGACTTGAAGAACG
GAGGCCGTTTCGTGGCGTTGAAGCGCGTGCGGGTGCAGACCGGCGAGGAGGGCATGCCGCTCTCCACCAT
CCGCGAGGTGGCGGTGCTGAGGCACCTGGAGACCTTCGAGCACCCCAACGTGGTCAGGTTGTTTGATGTG
TGCACAGTGTCACGAACAGACAGAGAAACCAAACTAACTTTAGTGTTTGAACATGTCGATCAAGACTTGA
CCACTTACTTGGATAAAGTTCCAGAGCCTGGAGTGCCCACTGAAACCATAAAGGATATGATGTTTCAGCT
TCTCCGAGGTCTGGACTTTCTTCATTCACACCGAGTAGTGCATCGCGATCTAAAACCACAGAACATTCTG
GTGACCAGCAGCGGACAAATAAAACTCGCTGACTTCGGCCTTGCCCGCATCTATAGTTTCCAGATGGCTC
TAACCTCAGTGGTCGTCACGCTGTGGTACAGAGCACCCGAAGTCTTGCTCCAGTCCAGCTACGCCACCCC
CGTGGATCTCTGGAGTGTTGGCTGCATATTTGCAGAAATGTTTCGTAGAAAGCCTCTTTTTCGTGGAAGT
TCAGATGTTGATCAACTAGGAAAAATCTTGGACGTGATTGGACTCCCAGGAGAAGAAGACTGGCCTAGAG
ATGTTGCCCTTCCCAGGCAGGCTTTTCATTCAAAATCTGCCCAACCAATTGAGAAGTTTGTAACAGATAT
CGATGAACTAGGCAAAGACCTACTTCTGAAGTGTTTGACATTTAACCCAGCCAAAAGAATATCTGCCTAC
AGTGCCCTGTCTCACCCATACTTCCAGGACCTGGAAAGGTGCAAAGAAAACCTGGATTCCCACCTGCCGC
CCAGCCAGAACACCTCGGAGCTGAATACAGCCTGAGGCCTCAGCAGCCGCCTTAAGCTGATCCTGCGGAG
AACACCCTTGGTGGCTTATGGGTCCCCCTCAGCAAGCCCTACAGAGCTGTGGAGGATTGCTATCTGGAGG
CCTTCCAGCTGCTGTCTTCTGGACAGGCTCTGCTTCTCCAAGGAAACCGCCTAGTTTACTGTTTTGAAAT
CAATGCAAGAGTGATTGCAGCTTTATGTTCATTTGTTTGTTTGTTTGTCTGTTTGTTTCAAGAACCTGGA
AAAATTCCAGAAGAAGAGAAGCTGCTGACCAATTGTGCTGCCATTTGATTTTTCTAACCTTGAATGCTGC
CAGTGTGGAGTGGGTAATCCAGGCACAGCTGAGTTATGATGTAATCTCTCTGCAGCTGCCGGGCCTGATT
TGGTACTTTTGAGTGTGTGTGTGCATGTGTGTGTGTGTGTGTGTGTGTGTGTGTGTGTATGTGAGAGATT
CTGTGATCTTTTAAAGTGTTACTTTTTGTAAACGACAAGAATAATTCAATTTTAAAGACTCAAGGTGGTC
AGTAAATAACAGGCATTTGTTCACTGAAGGTGATTCACCAAAATAGTCTTCTCAAATTAGAAAGTTAACC
CCATGTCCTCAGCATTTCTTTTCTGGCCAAAAGCAGTAAATTTGCTAGCAGTAAAAGATGAAGTTTTATA
CACACAGCAAAAAGGAGAAAAAATTCTAGTATATTTTAAGAGATGTGCATGCATTCTATTTAGTCTTCAG
AATGCTGAATTTACTTGTTGTAAGTCTATTTTAACCTTCTGTATGACATCATGCTTTATCATTTCTTTTG
GAAAATAGCCTGTAAGCTTTTTATTACTTGCTATAGGTTTAGGGAGTGTACCTCAGATAGATTTTAAAAA
AAAGAATAGAAAGCCTTTATTTCCTGGTTTGAAATTCCTTTCTTCCCTTTTTTTGTTGTTGTTATTGTTG
TTTGTTGTTGTTATTTTGTTTTTGTTTTTAGGAATTTGTCAGAAACTCTTTCCTGTTTTGGTTTGGAGAG
TAGTTCTCTCTAACTAGAGACAGGAGTGGCCTTGAAATTTTCCTCATCTATTACACTGTACTTTCTGCCA
CACACTGCCTTGTTGGCAAAGTATCCATCTTGTCTATCTCCCGGCACTTCTGAAATATATTGCTACCATT
GTATAACTAATAACAGATTGCTTAAGCTGTTCCCATGCACCACCTGTTTGCTTGCTTTCAATGAACCTTT
CATAAATTCGCAGTCTCAGCTTATGGTTTATGGCCTCGATTCTGCAAACCTAACAGGGTCACATATGTTC
TCTAATGCAGTCCTTCTACCTGGTGTTTACTTTTGTTACCTAAATAATGAGTAGGATCTTGTTTTGTTTT
ATCACCAGCACACAGATTGCTATAAACTGTTACTTTGTGAATTACATTTTTATAGAAGATATTTTCAGTG
TCTTTACCTGAGGGTATGTCTTTAGCTATGTTTTAGGGCCATACATTTACTCTATCAAATGATCTTTTCT
CCATCCCCCAGGCTGTGCTTATTTCTAGTGCCTTGTGCTCACTCCTGCTCTCTACAGAGCCAGCCTGGCC
TGGGCATTGTAAACAGCTTTTCCTTTTTCTCTTACTGTTTTCTCTACAGTCCTTTATATTTCATACCATC
TCTGCCTTATAAGTGGTTTAGTGCTCAGTTGGCTCTAGTAACCAGAGGACACAGAAAGTATCTTTTGGAA
AGTTTAGCCACCTGTGCTTTCTGACTCAGAGTGCATGCAACAGTTAGATCATGCAACAGTTAGATTATGT
TTAGGGTTAGGATTTTCAAAGAATGGAGGTTGCTGCACTCAGAAAATAATTCAGATCATGTTTATGCATT
ATTAAGTTGTACTGAATTCTTTGCAGCTTAATGTGATATATGACTATCTTGAACAAGAGAAAAAACTAGG
AGATGTTTCTCCTGAAGAGCTTTTGGGGTTGGGAACTATTCTTTTTTAATTGCTGTACTACTTAACATTG
TTCTAATTCAGTAGCTTGAGGAACAGGAACATTGTTTTCTAGAGCAAGATAATAAAGGAGATGGGCCATA
CAAATGTTTTCTACTTTCGTTGTGACAACATTGATTAGGTGTTGTCAGTACTATAAATGCTTGAGATATA
ATGAATCCACAGCATTCAAGGTCAGGTCTACTCAAAGTCTCACATGGAAAAGTGAGTTCTGCCTTTCCTT
TGATCGAGGGTCAAAATACAAAGACATTTTTGCTAGGGCCTACAAATTGAATTTAAAAACTCACTGCACT
GATTCATCTGAGCTTTTTGGTTAGTATTCATGGCTAGAGTGAACATAGCTTTAGTTTTTGCTGTTGTAAA
AGTGTTTTCATAAGTTCACTCAAGAAAAATGCAGCTGTTCTGAACTGGAATTTTTCAGCATTCTTTAGAA
TTTTAAATGAGTAGAGAGCTCAACTTTTATTCCTAGCATCTGCTTTTGACTCATTTCTAGGCAGTGCTTA
TGAAGAAAAATTAAAGCACAAACATTCTGGCATTCAATCGTTGGCAGATTATCTTCTGATGACACAGAAT
GAAAGGGCATCTCAGCCTCTCTGAACTTTGTAAAAATCTGTCCCCAGTTCTTCCATCGGTGTAGTTGTTG
CATTTGAGTGAATACTCTCTTGATTTATGTATTTTATGTCCAGATTCGCCATTTCTGAAATCCAGATCCA
ACACAAGCAGTCTTGCCGTTAGGGCATTTTGAAGCAGATAGTAGAGTAAGAACTTAGTGACTACAGCTTA
TTCTTCTGTAACATATGGTTTCAAACATCTTTGCCAAAAGCTAAGCAGTGGTGAACTGAAAAGGGCATAT
TGCCCCAAGGTTACACTGAAGCAGCTCATAGCAAGTTAAAATATTGTGACAGATTTGAAATCATGTTTGA
ATTTCATAGTAGGACCAGTACAAGAATGTCCCTGCTAGTTTCTGTTTGATGTTTGGTTCTGGCGGCTCAG
GCATTTTGGGAACTGTTGCACAGGGTGGAGTCAAAACAACCTACATATAAAAAGAGAAAAAGAGAAACTT
GTCCATTTAGCTTTCATAAGAAATCCCATGGCAAAGGGTAATAAAAAGGACCTAATCTTAAAAATACAAT
TTCTAAGCACTTGTAAGAACCCAGTGGGTTGGAGCCTCCCACTTTGTCCCTCCTTTGAAGTGGATGGGAA
CTCAAGGTGCAAAGAACCTGTTTTGGAAGAAAGCTTGGGGCCATTTCAGCCCCCTGTATTCTCATGATTT
TCTCTCAGGAAGCACACACTGTGAATGGCAGACTTTTCATTTAGCCCCAGGTGACTTACTAAAAATAGTT
GAAAATTATTCACCTAAGAATAGAATCTCAGCATTGTGTTAAATAAAAATGAAAGCTTTAGAAGGCATGA
GATGTTCCTATCTTAAATAAAGCATGTTTCTTTTCTATAGAGAAATGTATAGTTTGACTCTCCAGAATGT
ACTATCCATCTTGATGAGAAAACTCTTAAATAGTACCAAACATTTTGAACTTTAAATTATGTATTTAAAG
TGAGTGTTTAAGAAACTGTAGCTGCTTCTTTTACAAGTGGTGCCTATTAAAGTCAGTAATGGCCATTATT
GTTCCATTGTGGAAATTAAATTATGTAAGCTTCCTAATATCATAAACATATTAAAATTCTTCTAAAATAT
TGCTTTTCTTTTAAGTGACAATTTGACTATTCTTATGATAAGCACATGAGAGTGTCTTACATTTTCCAAA
AGCAGGCTTTAATTGCATAGTTGAGTCTAGGAAAAAATAATGTTAAAAGTGAATATGCCACCATAATTAC
TTAATTATGTTAGTATAGAAACTACAGAATATTTACCCTGGAAAGAAAATATTGGAATGTTATTATAAAC
TCTTAGATATTTATATAATTCAAAAGAATGCATGTTTCACATTGTGACAGATAAAGATGTATGATTTCTA
AGGCTTTAAAAATTATTCATAAAACAGTGGGCAATAGATAAAGGAAATTCTGGAGAAAATGAAGGTATTT
AAAGGGTAGTTTCAAAGCTATATATATTTTGAAGGATATATTCTTTATGAACAAATATATTGTAAAAATT
TATACTAAGGTCATCTGGTAACTGTGGGATTAATATGGTCGAAAACAAATGTTATGGAGAAGCTGTCCCA
AGCAAACTAAATTACCTGTACTTTTTTCCCATTTCAAGGGAAGAGGCAACCACATGAAGCAATACTTCTT
ACACATGCCTAAGAACGTTCATTGAAAAAATAAATTTTTAAAAGGCATGTGTTTCCTATGCCACCAATAC
TTTTGAAAAATTGTGAACCTTACCCAAAACCATTTATCATGTCCATTAAGTATATTTGGGTATATAATTA
GGAAGATATTTACATGTTCCATCTCCACAGTGGAAAAACTTATTGAGGCTACCAAAGTGTGCCAAGAAAT
GTAAGTCCTTAGAGTAATTAGAAATGCTGTTTTCCTCAAAAGCATGAGAAACTAGCATTTTCATTTCTTA
TTTACTCCCTTTCTATATCAATGCAATTCACAACCCAATTTTAATACATCCCTATATCTCAAGCATTTCT
ATCTTGTACTTTTTCAGAAAATAAACCAAAAATAATCCTTTGGTCTCTCTATCTTCTGACCTTTGTAAGC
AACAGAAATGTAAAAACAGAAGGGGTCCAATTTTTACACGTTTTTTTCTCAAGTAGCCTTTCTGGGGATT
TTTATTTTCTTAATGAAGTGCCAATCAGCTTTTCAAAATGTTTTCTATTTCTCAGCATTTCCAGGAAGTG
ATAACGTTTAGCTAAATGAGTAGAAGTGGACTTCCTTCAACATATTGTTACCTTGTCTAGCCTTAGGAAG
AAAACAAGAGCCACCTGAAAATAAATACAGGCTCTTTTCGAGCATCTGCTGAAATACTGTTACAGCAATT
TGAAGTTGATGTGGTAGGAAAGGAAGGTGACTTTTCTTGCAAAAGTCTTTCTAAACATTCACACTGTCCT
AAGAGATGAGCTTTCTTGTTTTATTCCGGTATATTCCACAAGGTGGCACTTTTAGAGAAAAACAAATCTG
ATGAAGACTAAAGAGGTACTTCTAAAAGAGATTTCATTCTAACTTTATTTTTCTGCGCATATTTAACTCT
TTCCTAGCACTTGTTTTTTGGGATGATTAATAGTCTCTATAATGTTCTGTAACTTCAATATTTTACTTGT
TACCTAGGTTCTGAACAATTGTCTGCAAATAAATTGTTCTTAAGGATGGATAATACACCCATTTTGATCA
TTTAAGTAAAGAAAGCCTAGTCATTCATTCAGTCAAGAAAAAATTTTTGAAGTACCCAGTTACCTTACTT
TTCTAGATTAAAACAGGCTTAGTTACTAAAAAGGCAGTCCTCATCTGTGAACAGGATAGTTTCGTTAGAA
GTATAAAACTCCTTTAGTGGCCCCAGTTAAAACACACATACCCTCTCTGCTGCTTTCAAATTCCCTAGCA
TGGTGGCCTTTCAACATTGATTAAATTTTAAAATCCTAATTTAAAGATCAGGTGAGCAAAATGAGTAGCA
CATCAGTAATTCAGTAGACAAAACTTTTGTCTGAAAAATTGCTGTATTGAAACAGAGCCCTAAAATACCA
AAAGACCAGGTAATTTTAACATTTGTGGAATCACAAATGTAAATTCATAAGAAGCTCTAATTAAAAAAAA
AAAGTCTGAAGTATATGAGCATAACAACTTAGGAGTGTGTCTACATACTTAACTTTTGAAGTTTTTTGGC
AACTTTATATACTTTTTTTAAATTTACAAGTCTACTTAAAGACTTCTTATACCCCAAATGATTAAGTTAA
TTTTAGAGGTCACCTTTCTCACAGCAGTGTCACTTGAAATTTAGTAGGGAAGGATATTGCAGTATTTTTC
AGTTTCCTTAGCACAGCACCACAGAAAGCAGCTTATTCCTTTTGAGTGGCAGACACTCGACGGTGCCTGC
CCAACTTTCCTCCTGAGTGGCAAGCAGATGAGTCTCAGTAATTCATACTGAACCAAAATGCCACATACAC
TAGGGGCAGTCAGAAACTGGCTGAGAAATCCCCCGCCTCATTCGCCCCTCTGCTCCCAGGAACTAGAGTC
CAGTTAAAGCCCCTATGCGAAAGGCCGAATTCCACCCCAGGGTTTGTTATAACAGTGGCCAGTCTGAACC
CCATTTGCTCGTGCTCAAAACTTGATTCCCACTTGAAAGCCTTCCGGGCGCGCTGCCTCGTTGCCCCGCC
CCTTTGGCAGGAGAGAGGCAGTGGGCGAGGCCGGGCTGGGGCCCCGCCTCCCACTCACCTGCCGGTGCCT
GAAATTATGTGCGGCCCCGCGGGCTGCTTTCCGAGGTCAGAGTGCCCTGCTGCTGTCTCAGAGGCATCTG
TTCTGCAAATCTTAGGAAGAAAAATGTCCCTAGTAGCAAACGGGTGTCTTCTGTGCATAAATAAGTACAA
CACAATTCTCCGAAAGTTCGGGTAAAAAGAGATGCGGTAGCAGCTGCCCTGTGTGAAGCTGTCTACCCCG
CATCTCTCAGGCGCTAAGCTCAGTTTTTGTTTTTGTTTTTGTTTTTTTAAAGAAAAGATGTATAATTGCA
GGAATTTTTTTTTATTTTTTTATTTTCCATCATTCTATATATGTGATGGTGAAAGATATGCCTGGAAAAG
TTTTGTTTTGAAAAGTTTATTTTCTGCTTCGTCTTCAGTTGGCAAAAGCTCTCAATTCTTTAGCTTCCAG
TTTCTTTTCTCTCTTTTTCTTTGTTAGGTAATTAAAGGTATGTAAACAAATTATCTCATGTAGCAGGGGA
TTTTCATGTTGAGAGGAATCTTCCGTGTGAGTTGTTTGGTCACACAAATAACCCTTTCTCAATTTTAGGA
GTTTGGATTGTCAAATGTAGGTTTTTCTCAAAGGGGGCATATAACTACATATTGACTGCCAAGAACTATG
ACTGTAGCACTAATCAGCACACATAGAGCCACACAATTATTTAATTTCTAACTCTCTGTGGTCCCTAGAA
AAATTCCGTTGATGTGCTTAGGTTAAAGTTCTGAAGATACCCGTTGTACCCTTACTTGAAAGTTTCTAAT
CTTAAGTTTTATGAAATGCAATAATATGTATCAGCTAGCAATATTTCTGTGATCACCAACAACTCTCAGT
TTGATCTTAAAGTCTGAATAATAAAACAAATCCCAGCAGTAATACATTTCTTAAACCTCACAGTGCATGA
TATATCTTTTCATTCTGATCCTGTGTTTGCAAAAATATACACATGTATATCATAGTTCCTCACTTTTTAT
TCATTTGTTTTCCTATTACCTGTAGTAAATATATTAGTTAGTACATGGAATTTATAGCATCAGCTACCCC
CAGGAACAGCACCTGACAGGCGGGGGATTTTTTTTCAAGTTGTTCTACATTTGCATAAATTATTTCTATT
ATTATTCATGTATGTTATTTATTTCTGAATCACACTAGTCCTGTGAAAGTACAACTGAAGGCAGAAAGTG
TTAGGATTTTGCATCTAATGTTCATTATCATGGTATTGATGGACCTAAGAAAATAAAAATTAGACTAAGC
CCCCAAATAAGCTGCATGCATTTGTAACATGATTAGTAGATTTGAATATATAGATGTAGTATTTTGGGTA
TCTAGGTGTTTTATCATTATGTAAAGGAATTAAAGTAAAGGACTTTGTAGTTGTTTTTATTAAATATGCA
TATAGTAGAGTGCAAAAATATAGCAAAAATAAAAACTAAAGGTAGAAAAGCATTTTAGATATGCCTTAAT
TTAGAAACTGTGCCAGGTGGCCCTCGGAATAGATGCCAGGCAGAGACCAGTGCCTGGGTGGTGCCTCCTC
TTGTCTGCCCTCATGAAGAAGCTTCCCTCACGTGATGTAGTGCCCTCGTAGGTGTCATGTGGAGTAGTGG
GAACAGGCAGTACTGTTGAGAGGAGAGCAGTGTGAGAGTTTTTCTGTAGAAGCAGAACTGTCAGCTTGTG
CCTTGAGGCTTCCAGAACGTGTCAGATGGAGAAGTCCAAGTTTCCATGCTTCAGGCAACTTAGCTGTGTA
CAGAAGCAATCCAGTGTGGTAATAAAAAGCAAGGATTGCCTGTATAATTTATTATAAAATAAAAGGGATT
TTAACAACCAACAATTCCCAACACCTCAAAAGCTTGTTGCATTTTTTGGTATTTGAGGTTTTTATCTGAA
GGTTAAAGGGCAAGTGTTTGGTATAGAAGAGCAGTATGTGTTAAGAAAAGAAAAATATTGGTTCACGTAG
AGTGCAAATTAGAACTAGAAAGTTTTATACGATTATCATTTTGAGATGTGTTAAAGTAGGTTTTCACTGT
AAAATGTATTAGTGTTTCTGCATTGCCATAGGGCCTGGTTAAAACTTTCTCTTAGGTTTCAGGAAGACTG
TCACATACAGTAAGCTTTTTTCCTTCTGACTTATAATAGAAAATGTTTTGAAAGTAAAAAAAAAAAATCT
AATTTGGAAATTTGACTTGTTAGTTTCTGTGTTTGAAATCATGGTTCTAGAAATGTAGAAATTGTGTATA
TCAGATACTCATCTAGGCTGTGTGAACCAGCCCAAGATGACCAACATCCCCACACCTCTACATCTCTGTC
CCCTGTATCTCTTCCTTTCTACCACTAAAGTGTTCCCTGCTACCATCCTGGCTTGTCCACATGGTGCTCT
CCATCTTCCTCCACATCATGGACCACAGGTGTGCCTGTCTAGGCCTGGCCACCACTCCCAACTTGACCTA
GCCACATTCATCTAGAGATGGTTCCTGATGCTGGGCACAGACTGTGCTCATGGCACCCATTAGAAATGCC
TCTAGCATCTTTGTATGCATCTTGATTTTTAAACCAAGTCATTGTACAGAGCATTCAGTTTTGGCTGTGG
TACCAAGAGAAAAACTAATCAAGAATATAAACCACATTCCAGGCTGCTGTTTTCTCTCCATCTACAGGCC
ACACTTTTACTGTATTTCTTCATACTTGAAATTCATTCTGCTATTTTCATATCAGGGTACAGACTTATAA
GGGTGCATGTTCCTTAAAGGTGCATAATTATTCTTATTCCGTTTGCTTATATTGCTACAGAATGCTCTGT
TTTGGTGCTTTGAGTTCTGCAGACCCAAGAAGCAGTGTGGAAATTCACTGCCTGGGACACAGTCTTATAA
GAATGTTGGCAGGTGACTTTGTATCAGATGTTGCTTCTCTTTTCTCTGTACACAGATTGAGAGTTACCAC
AGTGGCCTGTCGGGTCCACCCTGTGGGTGCAGCACAGCTCTCTGAAAGCAAGAACCTTCCTACCTATTCT
AACGTTTTTGCCCTCTAAGAAAAATGGCCTCAGGTATGGTATAGACATAGCAAGAGGGGAAGGGCTGTCT
CACTCTAGCAACCATCCCTCCATTACACACAGAAAGCCCTCTTGAAGCAAAAGAAGAAGAAAGAAAGAAA
GCTTATCTCTAAGGCTACTGTCTTCAGAATGCTCTGAGCTGAATGCTCTTGCTCCTTTCCCAAGAGGCAG
ATGAAAATATAGCCAGTTTATCTATACCCTTCCTATCTGAGGAGGAGAATAGAAAAGTAGGGTAAATATG
TAACGTAAAATATGTCATTCAAGGACCACCAAAACTTTAAGTACCCTATCATTAAAAATCTGGTTTTAAA
AGTAGCTCAAGTAAGGGATGCTTTGTGACCCAGGGTTTCTGAAGTCAGATAGCCATTCTTACCTGCCCCT
TACTCTGACTTATTGGGAAAGGGAGAACTGCAGTGGTGTTTCTGTTGCAGTGGCAAAGGTAACATGTCAG
AAAATTCAGAGGGTTGCATACCAATAATCCTTTGGAAACTGGATGTCTTACTGGGTGCTAGAATGAAAAT
GTAGGTATTTATTGTCAGATGATGAAGTTCATTGTTTTTTTCAAAATTGGTGTTGAAATATCACTGTCCA
ATGTGTTCACTTATGTGAAAGCTAAATTGAATGAGGCAAAAAGAGCAAATAGTTTGTATATTTGTAATAC
CTTTTGTATTTCTTACAATAAAAATATTGGTAGCAAATAAAAATAATAAAAACAATAACTTTAAACTGCT
TTCTGGAGATGAATTACTCTCCTGGCTATTTTCTTTTTTACTTTAATGTAAAATGAGTATAACTGTAGTG
AGTAAAATTCATTAAATTCCAAGTTTTAGCAGAA

By “CDK4/6 inhibitor” is meant an agent that reduces the activity or expression of a cyclin dependent kinase 4 or cyclin dependent kinase 6 polypeptide. Exemplary CDK4/6 inhibitors are listed in Table 6. In some embodiments, the CDK4/6 inhibitor is abemaciclib, AT7519, CINK4, flavopiridol, palbociclib, ribociclib, or a pharmaceutically acceptable salt thereof.

The term “CERES” refers to an analytic method that estimates gene-dependency levels from CRISPR-Cas9 essentiality screens while accounting for the anti-proliferative effect of Cas9-mediated DNA cleavage. CERES is described further, e.g., in Meyers R M, et al. “Computational correction of copy number effect improves specificity of CRISPR-Cas9 essentiality screens in cancer cells.” Nat Genet. 2017; 49(12):1779-1784, the teachings of which are incorporated herein by reference in its entirety.

By “chemotherapeutic agent” is meant any agent that inhibits cancer cell proliferation, inhibits cancer cell survival, increases cancer cell death, that inhibits and/or stabilizes tumor growth, or that is otherwise useful in the treatment of cancer. In embodiments, chemotherapeutic agents provided herein can be used in combination with a CDK4/6 inhibitor (e.g., abemaciclib, AT7519, CINK4, flavopiridol, palbociclib, and/or ribociclib), a c-Met inhibitor (e.g., AMG337, BMS 777607/ASLAN002, cabozantinib, capmatinib, crizotinib, emibetuzumab, ficlatuzumab, foretinib, glesatinib, onartuzumab, rilotumumab, tepotinib, tivantinib, and/or volitinib), an EGFR inhibitor (e.g., Erlotinib, Osimertinib, Neratinib, Gefitinib, Cetuximab, Panitumumab, Dacomitinib, Lapatinib, Necitumumab, Mobocertinib, and/or Vandetanib), a PD-1/PD-L1 checkpoint inhibitor (e.g., atezolizumab, avelumab, BMS-936559, MDX-1105, cemiplimab, durvalumab, nivolumab, and/or pembrolizumab), and/or a TGF-beta inhibitor (e.g., Galunisertib, Vactosertib, Trabedersen, ISTH0036, Fresolimumab, Disitertide, Lucanix™, and/or Gemogenovatucel-T). Exemplary chemotherapeutic agents are listed in Tables 4-6.

In embodiments, chemotherapeutic agents include, but are not limited to: 5-fluorouracil, abatacept, abemaciclib, adagrasib, afatinib, albumin-bound paclitaxel, altretamine, amsacrine, AMG337, atezolizumab, AT7519, bevacizumab, BMS 777607/ASLAN002, busulfan, cabozantinib, capmatinib, canertinib, carboplatin, ceritinib, CINK4, cisplatin, colchicine, crizotinib, cyclophosphamide chlorambucil, dabrafenib, dacarbazine, docetaxel, durvalumab, emibetuzumab, epothilone B, erlotinib, estramustine phosphate, etoposide (VP-16), ficlatuzumab, flavopiridol, foretinib, gefitinib, gemcitabine, glesatinib, hexamethylmelamine, ifosfamide, imatinib, iproplatin, ipilimumab, irinotecan, leflunomide, leucovorin, lobaplatin, lomustine, mekinist, mechlorethamine, nolatrexed, norelin, onartuzumab, ormaplatin, oxaliplatin, paclitaxel, palbociclib, pembrolizumab, pemetrexed, procarbazine, ramucirumab, ribociclib, rilotumumab, rituximab, satraplatin, semustine, sotorasib squalamine, spiroplatin, streptozocin, tafinlar, temozolomide, tepotinib, tetraplatin, tezacitabine, thiotepa, tipifamib, tivantinib, topotecan, trametinib, trastuzumab, vatalanib, vinblastine, vinflunine, vindesine, vinorelbine, and volitinib. Such agents can be used alone or in combination with another agent described herein, such as a c-MET inhibitor.

In some embodiments, such agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. One of skill in the art can readily identify a chemotherapeutic agent of use in a method for treating a cancer described herein (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). In some embodiments of any of the aspects, the combination of agents provided herein decrease cancer cell proliferation or survival by at least 10%, 20%, 30%, 40%, 50%, 60%, 70%, 80%, 90%, 95%, or 100%, and includes inducing cell death (apoptosis) in a cell or cells within a cell mass.

By “c-mesenchymal-epithelial transition factor (MET; c-MET) polypeptide” is meant a receptor tyrosine kinase or a fragment thereof that has tyrosine kinase activity and that has at least about 85% or greater amino acid sequence identity to NCBI Gene ID: 4233; NCBI Reference Sequence: NP_000236.2. An exemplary human c-Met amino acid sequence is provided below:

>NP_000236.2 hepatocyte growth factor receptor
isoform b preproprotein [Homo sapiens]
(SEQ ID NO. 5)
MKAPAVLAPGILVLLFTLVQRSNGECKEALAKSEMNVNMKYQLPNFTAET
PIQNVILHEHHIFLGATNYIYVLNEEDLQKVAEYKTGPVLEHPDCFPCQD
CSSKANLSGGVWKDNINMALVVDTYYDDQLISCGSVNRGTCQRHVFPHNH
TADIQSEVHCIFSPQIEEPSQCPDCVVSALGAKVLSSVKDRFINFFVGNT
INSSYFPDHPLHSISVRRLKETKDGFMFLTDQSYIDVLPEFRDSYPIKYV
HAFESNNFIYFLTVQRETLDAQTFHTRIIRFCSINSGLHSYMEMPLECIL
TEKRKKRSTKKEVENILQAAYVSKPGAQLARQIGASLNDDILFGVFAQSK
PDSAEPMDRSAMCAFPIKYVNDFFNKIVNKNNVRCLQHFYGPNHEHCFNR
TLLRNSSGCEARRDEYRTEFTTALQRVDLFMGQFSEVLLTSISTFIKGDL
TIANLGTSEGRFMQVVVSRSGPSTPHVNFLLDSHPVSPEVIVEHTLNQNG
YTLVITGKKITKIPLNGLGCRHFQSCSQCLSAPPFVQCGWCHDKCVRSEE
CLSGTWTQQICLPAIYKVFPNSAPLEGGTRLTICGWDFGFRRNNKFDLKK
TRVLLGNESCTLTLSESTMNTLKCTVGPAMNKHFNMSIIISNGHGTTQYS
TFSYVDPVITSISPKYGPMAGGTLLTLTGNYLNSGNSRHISIGGKTCTLK
SVSNSILECYTPAQTISTEFAVKLKIDLANRETSIFSYREDPIVYEIHPT
KSFISGGSTITGVGKNLNSVSVPRMVINVHEAGRNFTVACQHRSNSEIIC
CTTPSLQQLNLQLPLKTKAFFMLDGILSKYFDLIYVHNPVFKPFEKPVMI
SMGNENVLEIKGNDIDPEAVKGEVLKVGNKSCENIHLHSEAVLCTVPNDL
LKLNSELNIEWKQAISSTVLGKVIVQPDQNFTGLIAGVVSISTALLLLLG
FFLWLKKRKQIKDLGSELVRYDARVHTPHLDRLVSARSVSPTTEMVSNES
VDYRATFPEDQFPNSSQNGSCRQVQYPLTDMSPILTSGDSDISSPLLQNT
VHIDLSALNPELVQAVQHVVIGPSSLIVHFNEVIGRGHFGCVYHGTLLDN
DGKKIHCAVKSLNRITDIGEVSQFLTEGIIMKDFSHPNVLSLLGICLRSE
GSPLVVLPYMKHGDLRNFIRNETHNPTVKDLIGFGLQVAKGMKYLASKKF
VHRDLAARNCMLDEKFTVKVADFGLARDMYDKEYYSVHNKTGAKLPVKWM
ALESLQTQKFTTKSDVWSFGVLLWELMTRGAPPYPDVNTFDITVYLLQGR
RLLQPEYCPDPLYEVMLKCWHPKAEMRPSFSELVSRISAIFSTFIGEHYV
HVNATYVNVKCVAPYPSLLSSEDNADDEVDTRPASFWETS

By “c-MET polynucleotide” is meant a nucleic acid molecule encoding a C-MET polypeptide. An exemplary c-MET polynucleotide sequence is provided at NM_000245, which is reproduced below.

>NM_000245.4 Homo sapiens MET proto-oncogene, receptor tyrosine kinase (MET),
transcript variant 2, mRNA
(SEQ ID NO. 6)
AGACACGTGCTGGGGCGGGCAGGCGAGCGCCTCAGTCTGGTCGCCTGGCGGTGCCTCCGGCCCCAACGCG
CCCGGGCCGCCGCGGGCCGCGCGCGCCGATGCCCGGCTGAGTCACTGGCAGGGCAGCGCGCGTGTGGGAA
GGGGCGGAGGGAGTGCGGCCGGCGGGCGGGCGGGGCGCTGGGCTCAGCCCGGCCGCAGGTGACCCGGAGG
CCCTCGCCGCCCGCGGCGCCCCGAGCGCTTTGTGAGCAGATGCGGAGCCGAGTGGAGGGCGCGAGCCAGA
TGCGGGGCGACAGCTGACTTGCTGAGAGGAGGGGGGAGGCGCGGAGCGCGCGTGTGGTCCTTGCGCCGC
TGACTTCTCCACTGGTTCCTGGGCACCGAAAGATAAACCTCTCATAATGAAGGCCCCCGCTGTGCTTGCA
CCTGGCATCCTCGTGCTCCTGTTTACCTTGGTGCAGAGGAGCAATGGGGAGTGTAAAGAGGCACTAGCAA
AGTCCGAGATGAATGTGAATATGAAGTATCAGCTTCCCAACTTCACCGCGGAAACACCCATCCAGAATGT
CATTCTACATGAGCATCACATTTTCCTTGGTGCCACTAACTACATTTATGTTTTAAATGAGGAAGACCTT
CAGAAGGTTGCTGAGTACAAGACTGGGCCTGTGCTGGAACACCCAGATTGTTTCCCATGTCAGGACTGCA
GCAGCAAAGCCAATTTATCAGGAGGTGTTTGGAAAGATAACATCAACATGGCTCTAGTTGTCGACACCTA
CTATGATGATCAACTCATTAGCTGTGGCAGCGTCAACAGAGGGACCTGCCAGCGACATGTCTTTCCCCAC
AATCATACTGCTGACATACAGTCGGAGGTTCACTGCATATTCTCCCCACAGATAGAAGAGCCCAGCCAGT
GTCCTGACTGTGTGGTGAGCGCCCTGGGAGCCAAAGTCCTTTCATCTGTAAAGGACCGGTTCATCAACTT
CTTTGTAGGCAATACCATAAATTCTTCTTATTTCCCAGATCATCCATTGCATTCGATATCAGTGAGAAGG
CTAAAGGAAACGAAAGATGGTTTTATGTTTTTGACGGACCAGTCCTACATTGATGTTTTACCTGAGTTCA
GAGATTCTTACCCCATTAAGTATGTCCATGCCTTTGAAAGCAACAATTTTATTTACTTCTTGACGGTCCA
AAGGGAAACTCTAGATGCTCAGACTTTTCACACAAGAATAATCAGGTTCTGTTCCATAAACTCTGGATTG
CATTCCTACATGGAAATGCCTCTGGAGTGTATTCTCACAGAAAAGAGAAAAAAGAGATCCACAAAGAAGG
AAGTGTTTAATATACTTCAGGCTGCGTATGTCAGCAAGCCTGGGGCCCAGCTTGCTAGACAAATAGGAGC
CAGCCTGAATGATGACATTCTTTTCGGGGTGTTCGCACAAAGCAAGCCAGATTCTGCCGAACCAATGGAT
CGATCTGCCATGTGTGCATTCCCTATCAAATATGTCAACGACTTCTTCAACAAGATCGTCAACAAAAACA
ATGTGAGATGTCTCCAGCATTTTTACGGACCCAATCATGAGCACTGCTTTAATAGGACACTTCTGAGAAA
TTCATCAGGCTGTGAAGCGCGCCGTGATGAATATCGAACAGAGTTTACCACAGCTTTGCAGCGCGTTGAC
TTATTCATGGGTCAATTCAGCGAAGTCCTCTTAACATCTATATCCACCTTCATTAAAGGAGACCTCACCA
TAGCTAATCTTGGGACATCAGAGGGTCGCTTCATGCAGGTTGTGGTTTCTCGATCAGGACCATCAACCCC
TCATGTGAATTTTCTCCTGGACTCCCATCCAGTGTCTCCAGAAGTGATTGTGGAGCATACATTAAACCAA
AATGGCTACACACTGGTTATCACTGGGAAGAAGATCACGAAGATCCCATTGAATGGCTTGGGCTGCAGAC
ATTTCCAGTCCTGCAGTCAATGCCTCTCTGCCCCACCCTTTGTTCAGTGTGGCTGGTGCCACGACAAATG
TGTGCGATCGGAGGAATGCCTGAGCGGGACATGGACTCAACAGATCTGTCTGCCTGCAATCTACAAGGTT
TTCCCAAATAGTGCACCCCTTGAAGGAGGGACAAGGCTGACCATATGTGGCTGGGACTTTGGATTTCGGA
GGAATAATAAATTTGATTTAAAGAAAACTAGAGTTCTCCTTGGAAATGAGAGCTGCACCTTGACTTTAAG
TGAGAGCACGATGAATACATTGAAATGCACAGTTGGTCCTGCCATGAATAAGCATTTCAATATGTCCATA
ATTATTTCAAATGGCCACGGGACAACACAATACAGTACATTCTCCTATGTGGATCCTGTAATAACAAGTA
TTTCGCCGAAATACGGTCCTATGGCTGGTGGCACTTTACTTACTTTAACTGGAAATTACCTAAACAGTGG
GAATTCTAGACACATTTCAATTGGTGGAAAAACATGTACTTTAAAAAGTGTGTCAAACAGTATTCTTGAA
TGTTATACCCCAGCCCAAACCATTTCAACTGAGTTTGCTGTTAAATTGAAAATTGACTTAGCCAACCGAG
AGACAAGCATCTTCAGTTACCGTGAAGATCCCATTGTCTATGAAATTCATCCAACCAAATCTTTTATTAG
TGGTGGGAGCACAATAACAGGTGTTGGGAAAAACCTGAATTCAGTTAGTGTCCCGAGAATGGTCATAAAT
GTGCATGAAGCAGGAAGGAACTTTACAGTGGCATGTCAACATCGCTCTAATTCAGAGATAATCTGTTGTA
CCACTCCTTCCCTGCAACAGCTGAATCTGCAACTCCCCCTGAAAACCAAAGCCTTTTTCATGTTAGATGG
GATCCTTTCCAAATACTTTGATCTCATTTATGTACATAATCCTGTGTTTAAGCCTTTTGAAAAGCCAGTG
ATGATCTCAATGGGCAATGAAAATGTACTGGAAATTAAGGGAAATGATATTGACCCTGAAGCAGTTAAAG
GTGAAGTGTTAAAAGTTGGAAATAAGAGCTGTGAGAATATACACTTACATTCTGAAGCCGTTTTATGCAC
GGTCCCCAATGACCTGCTGAAATTGAACAGCGAGCTAAATATAGAGTGGAAGCAAGCAATTTCTTCAACC
GTCCTTGGAAAAGTAATAGTTCAACCAGATCAGAATTTCACAGGATTGATTGCTGGTGTTGTCTCAATAT
CAACAGCACTGTTATTACTACTTGGGTTTTTCCTGTGGCTGAAAAAGAGAAAGCAAATTAAAGATCTGGG
CAGTGAATTAGTTCGCTACGATGCAAGAGTACACACTCCTCATTTGGATAGGCTTGTAAGTGCCCGAAGT
GTAAGCCCAACTACAGAAATGGTTTCAAATGAATCTGTAGACTACCGAGCTACTTTTCCAGAAGATCAGT
TTCCTAATTCATCTCAGAACGGTTCATGCCGACAAGTGCAGTATCCTCTGACAGACATGTCCCCCATCCT
AACTAGTGGGGACTCTGATATATCCAGTCCATTACTGCAAAATACTGTCCACATTGACCTCAGTGCTCTA
AATCCAGAGCTGGTCCAGGCAGTGCAGCATGTAGTGATTGGGCCCAGTAGCCTGATTGTGCATTTCAATG
AAGTCATAGGAAGAGGGCATTTTGGTTGTGTATATCATGGGACTTTGTTGGACAATGATGGCAAGAAAAT
TCACTGTGCTGTGAAATCCTTGAACAGAATCACTGACATAGGAGAAGTTTCCCAATTTCTGACCGAGGGA
ATCATCATGAAAGATTTTAGTCATCCCAATGTCCTCTCGCTCCTGGGAATCTGCCTGCGAAGTGAAGGGT
CTCCGCTGGTGGTCCTACCATACATGAAACATGGAGATCTTCGAAATTTCATTCGAAATGAGACTCATAA
TCCAACTGTAAAAGATCTTATTGGCTTTGGTCTTCAAGTAGCCAAAGGCATGAAATATCTTGCAAGCAAA
AAGTTTGTCCACAGAGACTTGGCTGCAAGAAACTGTATGCTGGATGAAAAATTCACAGTCAAGGTTGCTG
ATTTTGGTCTTGCCAGAGACATGTATGATAAAGAATACTATAGTGTACACAACAAAACAGGTGCAAAGCT
GCCAGTGAAGTGGATGGCTTTGGAAAGTCTGCAAACTCAAAAGTTTACCACCAAGTCAGATGTGTGGTCC
TTTGGCGTGCTCCTCTGGGAGCTGATGACAAGAGGAGCCCCACCTTATCCTGACGTAAACACCTTTGATA
TAACTGTTTACTTGTTGCAAGGGAGAAGACTCCTACAACCCGAATACTGCCCAGACCCCTTATATGAAGT
AATGCTAAAATGCTGGCACCCTAAAGCCGAAATGCGCCCATCCTTTTCTGAACTGGTGTCCCGGATATCA
GCGATCTTCTCTACTTTCATTGGGGAGCACTATGTCCATGTGAACGCTACTTATGTGAACGTAAAATGTG
TCGCTCCGTATCCTTCTCTGTTGTCATCAGAAGATAACGCTGATGATGAGGTGGACACACGACCAGCCTC
CTTCTGGGAGACATCATAGTGCTAGTACTATGTCAAAGCAACAGTCCACACTTTGTCCAATGGTTTTTTC
ACTGCCTGACCTTTAAAAGGCCATCGATATTCTTTGCTCTTGCCAAAATTGCACTATTATAGGACTTGTA
TTGTTATTTAAATTACTGGATTCTAAGGAATTTCTTATCTGACAGAGCATCAGAACCAGAGGCTTGGTCC
CACAGGCCACGGACCAATGGCCTGCAGCCGTGACAACACTCCTGTCATATTGGAGTCCAAAACTTGAATT
CTGGGTTGAATTTTTTAAAAATCAGGTACCACTTGATTTCATATGGGAAATTGAAGCAGGAAATATTGAG
GGCTTCTTGATCACAGAAAACTCAGAAGAGATAGTAATGCTCAGGACAGGAGCGGCAGCCCCAGAACAGG
CCACTCATTTAGAATTCTAGTGTTTCAAAACACTTTTGTGTGTTGTATGGTCAATAACATTTTTCATTAC
TGATGGTGTCATTCACCCATTAGGTAAACATTCCCTTTTAAATGTTTGTTTGTTTTTTGAGACAGGATCT
CACTCTGTTGCCAGGGCTGTAGTGCAGTGGTGTGATCATAGCTCACTGCAACCTCCACCTCCCAGGCTCA
AGCCTCCCGAATAGCTGGGACTACAGGCGCACACCACCATCCCCGGCTAATTTTTGTATTTTTTGTAGAG
ACGGGGTTTTGCCATGTTGCCAAGGCTGGTTTCAAACTCCTGGACTCAAGAAATCCACCCACCTCAGCCT
CCCAAAGTGCTAGGATTACAGGCATGAGCCACTGCGCCCAGCCCTTATAAATTTTTGTATAGACATTCCT
TTGGTTGGAAGAATATTTATAGGCAATACAGTCAAAGTTTCAAAATAGCATCACACAAAACATGTTTATA
AATGAACAGGATGTAATGTACATAGATGACATTAAGAAAATTTGTATGAAATAATTTAGTCATCATGAAA
TATTTAGTTGTCATATAAAAACCCACTGTTTGAGAATGATGCTACTCTGATCTAATGAATGTGAACATGT
AGATGTTTTGTGTGTATTTTTTTAAATGAAAACTCAAAATAAGACAAGTAATTTGTTGATAAATATTTTT
AAAGATAACTCAGCATGTTTGTAAAGCAGGATACATTTTACTAAAAGGTTCATTGGTTCCAATCACAGCT
CATAGGTAGAGCAAAGAAAGGGTGGATGGATTGAAAAGATTAGCCTCTGTCTCGGTGGCAGGTTCCCACC
TCGCAAGCAATTGGAAACAAAACTTTTGGGGAGTTTTATTTTGCATTAGGGTGTGTTTTATGTTAAGCAA
AACATACTTTAGAAACAAATGAAAAAGGCAATTGAAAATCCCAGCTATTTCACCTAGATGGAATAGCCAC
CCTGAGCAGAACTTTGTGATGCTTCATTCTGTGGAATTTTGTGCTTGCTACTGTATAGTGCATGTGGTGT
AGGTTACTCTAACTGGTTTTGTCGACGTAAACATTTAAAGTGTTATATTTTTTATAAAAATGTTTATTTT
TAATGATATGAGAAAAATTTTGTTAGGCCACAAAAACACTGCACTGTGAACATTTTAGAAAAGGTATGTC
AGACTGGGATTAATGACAGCATGATTTTCAATGACTGTAAATTGCGATAAGGAAATGTACTGATTGCCAA
TACACCCCACCCTCATTACATCATCAGGACTTGAAGCCAAGGGTTAACCCAGCAAGCTACAAAGAGGGTG
TGTCACACTGAAACTCAATAGTTGAGTTTGGCTGTTGTTGCAGGAAAATGATTATAACTAAAAGCTCTCT
GATAGTGCAGAGACTTACCAGAAGACACAAGGAATTGTACTGAAGAGCTATTACAATCCAAATATTGCCG
TTTCATAAATGTAATAAGTAATACTAATTCACAGAGTATTGTAAATGGTGGATGACAAAAGAAAATCTGC
TCTGTGGAAAGAAAGAACTGTCTCTACCAGGGTCAAGAGCATGAACGCATCAATAGAAAGAACTCGGGGA
AACATCCCATCAACAGGACTACACACTTGTATATACATTCTTGAGAACACTGCAATGTGAAAATCACGTT
TGCTATTTATAAACTTGTCCTTAGATTAATGTGTCTGGACAGATTGTGGGAGTAAGTGATTCTTCTAAGA
ATTAGATACTTGTCACTGCCTATACCTGCAGCTGAACTGAATGGTACTTCGTATGTTAATAGTTGTTCTG
ATAAATCATGCAATTAAAGTAAAGTGATGCAA.

By “c-Met inhibitor” is meant an agent that reduces the activity or expression of a c-Met polypeptide or polynucleotide. Exemplary c-Met inhibitors are listed in Table 4. In some embodiments of any of the aspects, the c-Met inhibitor is AMG337, BMS 777607/ASLAN002, cabozantinib, capmatinib, crizotinib, emibetuzumab, ficlatuzumab, foretinib, glesatinib, onartuzumab, rilotumumab, tepotinib, tivantinib, volitinib, or a pharmaceutically acceptable salt thereof.

By “combination therapy” is meant administration of two or more therapeutic agents in a coordinated fashion. In embodiments, combination therapy encompasses both co-administration (e.g., administration of a co-formulation or simultaneous administration of separate therapeutic compositions) and serial or sequential administration. In embodiments, administration of one therapeutic agent is conditioned in some way on administration of another therapeutic agent. For example, one therapeutic agent may be administered only after a different therapeutic agent has been administered and allowed to act for a prescribed period of time. In some embodiments, administration of the two components of the combination is separated by minutes, hours, or even days.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” or “detecting” refers to identifying the presence, absence or amount of an analyte to be detected.

The phrase “detecting or “diagnosing cancer” refers to determining the presence or absence of cancer or a precancerous condition in a subject.

The term “detectable” means a level of an analyte that can be measure or observed using standard techniques. Exemplary techniques for detecting RNA and/or DNA include, but are not limited to, differential display, RT (reverse transcriptase)-coupled polymerase chain reaction (PCR), Northern or Southern Blot, and/or RNase protection analyses.

By “disease” is meant any condition or disorder that damages or interferes with the normal function of a cell, tissue, or organ. Examples of diseases include but are not limited to lung cancer (e.g., lung adenocarcinoma (LUAD), S1, S2, S3, S4, and S5).

By “effective amount” is meant the amount of a required to ameliorate the symptoms of a disease (e.g., lung cancer) relative to an untreated patient. The effective amount of active compound(s) or agent(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “epidermal growth factor receptor (EGFR) polypeptide” is meant a protein or a fragment thereof having tyrosine kinase activity and having at least about 85% or greater amino acid sequence identity to GenBank Accession No. CAA25240.1, provided below. An exemplary human EGFR amino acid sequence is provided below:

>CAA25240.1 epidermal growth factor receptor
[Homo sapiens]
(SEQ ID NO. 7)
MRPSGTAGAALLALLAALCPASRALEEKKVCQGTSNKLTQLGTFEDHFLS
LQRMENNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGELLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCKLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA

By “epidermal growth factor receptor (EGFR) polynucleotide” is meant a nucleic acid molecule or fragment thereof encoding an EGFR polypeptide. The sequence of an exemplary EGFR polynucleotide is provided at GenBank Accession No. X00588.1, which is reproduced below.

>X00588.1: 187-3819 Human mRNA for precursor of epidermal growth factor
receptor
(SEQ ID NO. 8)
ATGCGACCCTCCGGGACGGCCGGGGCAGCGCTCCTGGCGCTGCTGGCTGCGCTCTGCCCGGCGAGTCGGG
CTCTGGAGGAAAAGAAAGTTTGCCAAGGCACGAGTAACAAGCTCACGCAGTTGGGCACTTTTGAAGATCA
TTTTCTCAGCCTCCAGAGGATGTTCAATAACTGTGAGGTGGTCCTTGGGAATTTGGAAATTACCTATGTG
CAGAGGAATTATGATCTTTCCTTCTTAAAGACCATCCAGGAGGTGGCTGGTTATGTCCTCATTGCCCTCA
ACACAGTGGAGCGAATTCCTTTGGAAAACCTGCAGATCATCAGAGGAAATATGTACTACGAAAATTCCTA
TGCCTTAGCAGTCTTATCTAACTATGATGCAAATAAAACCGGACTGAAGGAGCTGCCCATGAGAAATTTA
CAGGAAATCCTGCATGGCGCCGTGCGGTTCAGCAACAACCCTGCCCTGTGCAACGTGGAGAGCATCCAGT
GGCGGGACATAGTCAGCAGTGACTTTCTCAGCAACATGTCGATGGACTTCCAGAACCACCTGGGCAGCTG
CCAAAAGTGTGATCCAAGCTGTCCCAATGGGAGCTGCTGGGGTGCAGGAGAGGAGAACTGCCAGAAACTG
ACCAAAATCATCTGTGCCCAGCAGTGCTCCGGGCGCTGCCGTGGCAAGTCCCCCAGTGACTGCTGCCACA
ACCAGTGTGCTGCAGGCTGCACAGGCCCCCGGGAGAGCGACTGCCTGGTCTGCCGCAAATTCCGAGACGA
AGCCACGTGCAAGGACACCTGCCCCCCACTCATGCTCTACAACCCCACCACGTACCAGATGGATGTGAAC
CCCGAGGGCAAATACAGCTTTGGTGCCACCTGCGTGAAGAAGTGTCCCCGTAATTATGTGGTGACAGATC
ACGGCTCGTGCGTCCGAGCCTGTGGGGCCGACAGCTATGAGATGGAGGAAGACGGCGTCCGCAAGTGTAA
GAAGTGCGAAGGGCCTTGCCGCAAAGTGTGTAACGGAATAGGTATTGGTGAATTTAAAGACTCACTCTCC
ATAAATGCTACGAATATTAAACACTTCAAAAACTGCACCTCCATCAGTGGCGATCTCCACATCCTGCCGG
TGGCATTTAGGGGTGACTCCTTCACACATACTCCTCCTCTGGATCCACAGGAACTGGATATTCTGAAAAC
CGTAAAGGAAATCACAGGGTTTTTGCTGATTCAGGCTTGGCCTGAAAACAGGACGGACCTCCATGCCTTT
GAGAACCTAGAAATCATACGCGGCAGGACCAAGCAACATGGTCAGTTTTCTCTTGCAGTCGTCAGCCTGA
ACATAACATCCTTGGGATTACGCTCCCTCAAGGAGATAAGTGATGGAGATGTGATAATTTCAGGAAACAA
AAATTTGTGCTATGCAAATACAATAAACTGGAAAAAACTGTTTGGGACCTCCGGTCAGAAAACCAAAATT
ATAAGCAACAGAGGTGAAAACAGCTGCAAGGCCACAGGCCAGGTCTGCCATGCCTTGTGCTCCCCCGAGG
GCTGCTGGGGCCCGGAGCCCAGGGACTGCGTCTCTTGCCGGAATGTCAGCCGAGGCAGGGAATGCGTGGA
CAAGTGCAAGCTTCTGGAGGGTGAGCCAAGGGAGTTTGTGGAGAACTCTGAGTGCATACAGTGCCACCCA
GAGTGCCTGCCTCAGGCCATGAACATCACCTGCACAGGACGGGGACCAGACAACTGTATCCAGTGTGCCC
ACTACATTGACGGCCCCCACTGCGTCAAGACCTGCCCGGCAGGAGTCATGGGAGAAAACAACACCCTGGT
CTGGAAGTACGCAGACGCCGGCCATGTGTGCCACCTGTGCCATCCAAACTGCACCTACGGATGCACTGGG
CCAGGTCTTGAAGGCTGTCCAACGAATGGGCCTAAGATCCCGTCCATCGCCACIGGGATGGTGGGGGCCC
TCCTCTTGCTGCTGGTGGTGGCCCTGGGGATCGGCCTCTTCATGCGAAGGCGCCACATCGTTCGGAAGCG
CACGCTGCGGAGGCTGCTGCAGGAGAGGGAGCTTGTGGAGCCTCTTACACCCAGTGGAGAAGCTCCCAAC
CAAGCTCTCTTGAGGATCTTGAAGGAAACTGAATTCAAAAAGATCAAAGTGCTGGGCTCCGGTGCGTTCG
GCACGGTGTATAAGGGACTCTGGATCCCAGAAGGTGAGAAAGTTAAAATTCCCGTCGCTATCAAGGAATT
AAGAGAAGCAACATCTCCGAAAGCCAACAAGGAAATCCTCGATGAAGCCTACGTGATGGCCAGCGTGGAC
AACCCCCACGTGTGCCGCCTGCTGGGCATCTGCCTCACCTCCACCGTGCAACTCATCACGCAGCTCATGC
CCTTCGGCTGCCTCCTGGACTATGTCCGGGAACACAAAGACAATATTGGCTCCCAGTACCTGCTCAACTG
GTGTGTGCAGATCGCAAAGGGCATGAACTACTTGGAGGACCGTCGCTTGGTGCACCGCGACCTGGCAGCC
AGGAACGTACTGGTGAAAACACCGCAGCATGTCAAGATCACAGATTTTGGGCTGGCCAAACTGCTGGGTG
CGGAAGAGAAAGAATACCATGCAGAAGGAGGCAAAGTGCCTATCAAGTGGATGGCATTGGAATCAATTTT
ACACAGAATCTATACCCACCAGAGTGATGTCTGGAGCTACGGGGTGACCGTTTGGGAGTTGATGACCTTT
GGATCCAAGCCATATGACGGAATCCCTGCCAGCGAGATCTCCTCCATCCTGGAGAAAGGAGAACGCCTCC
CTCAGCCACCCATATGTACCATCGATGTCTACATGATCATGGTCAAGTGCTGGATGATAGACGCAGATAG
TCGCCCAAAGTTCCGTGAGTTGATCATCGAATTCTCCAAAATGGCCCGAGACCCCCAGCGCTACCTTGTC
ATTCAGGGGGATGAAAGAATGCATTTGCCAAGTCCTACAGACTCCAACTTCTACCGTGCCCTGATGGATG
AAGAAGACATGGACGACGTGGTGGATGCCGACGAGTACCTCATCCCACAGCAGGGCTTCTTCAGCAGCCC
CTCCACGTCACGGACTCCCCTCCTGAGCTCTCTGAGTGCAACCAGCAACAATTCCACCGTGGCTTGCATT
GATAGAAATGGGCTGCAAAGCTGTCCCATCAAGGAAGACAGCTTCTTGCAGCGATACAGCTCAGACCCCA
CAGGCGCCTTGACTGAGGACAGCATAGACGACACCTTCCTCCCAGTGCCTGAATACATAAACCAGTCCGT
TCCCAAAAGGCCCGCTGGCTCTGTGCAGAATCCTGTCTATCACAATCAGCCTCTGAACCCCGCGCCCAGC
AGAGACCCACACTACCAGGACCCCCACAGCACTGCAGTGGGCAACCCCGAGTATCTCAACACTGTCCAGC
CCACCTGTGTCAACAGCACATTCGACAGCCCTGCCCACTGGGCCCAGAAAGGCAGCCACCAAATTAGCCT
GGACAACCCTGACTACCAGCAGGACTTCTTTCCCAAGGAAGCCAAGCCAAATGGCATCTTTAAGGGCTCC
ACAGCTGAAAATGCAGAATACCTAAGGGTCGCGCCACAAAGCAGTGAATTTATTGGAGCATGA.

By “EGFR inhibitor” is meant an agent that reduces the activity or expression of an EGFR polypeptide. Non-limiting examples of EGFR inhibitors include Erlotinib, Osimertinib, Neratinib, Gefitinib, Cetuximab, Panitumumab, Dacomitinib, Lapatinib, Necitumumab, Mobocertinib, Vandetanib, and pharmaceutically acceptable salts thereof.

As used herein, the term “failed to respond to a prior therapy” or “refractory to a prior therapy” refers to a cancer that progressed despite treatment with the therapy.

By “fragment” is meant a portion of a polypeptide or nucleic acid molecule. This portion contains at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term “inhibiting cancer cell growth or proliferation” means decreasing a tumor cell's growth or proliferation by at least 10%, 20%, 30%, 40%, 50%, 60%, 70%, 80%, 90%, 95%, or 100%, and includes inducing cell death (apoptosis) in a cell or cells within a cell mass.

By “increase” is meant to alter positively. An increase may be by about or at least about 0.5%, 1%, 5%, 10%, 25%, 30%, 50%, 75%, or even by 100%.

The terms “isolated,” “purified,” or “biologically pure” refer to material that is free to varying degrees from components which normally accompany it as found in its native state. “Isolate” denotes a degree of separation from an original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding an additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In some embodiments the preparation is at least about 75%, at least about 90%, or at least about 99%, by weight, a polypeptide of the disclosure.

An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “lung cancer polynucleotide” is meant any nucleic acid molecule, or fragment thereof, whose expression is altered in connection with a lung cancer subtype described herein. Exemplary lung cancer polynucleotides are listed in Table 1.

By “lung cancer subtype” is meant a lung cancer or tumor identified as having characteristics associated with an S1, S2, S3, S4, or S5 lung cancer provided herein. For example, an S3 lung cancer features one or more markers selected from Table 2 and an S4 lung cancer features one or more markers selected from Table 3. The specific characteristics of each lung cancer subtype are discussed elsewhere below.

As used herein, the term “marker” can be used interchangeably with the term “biomarker” to refer to any analyte (e.g., protein or polynucleotide) having an alteration in expression level, structure, or activity that is associated with a disease or disorder (e.g., lung cancer) or a subtype of that disease (e.g., S1, S2, S3, S4, or S5 lung cancer subtype provided herein). For example, an S3 lung cancer features one or more markers selected from Table 2, an S4 lung cancer features one or more markers selected from Table 3, and an S2 lung cancer features one or more markers selected from Table 3B.

By “marker profile” is meant a characterization of the expression or expression level of two or more polypeptides or polynucleotides in a sample.

As used herein, “obtaining” as in “obtaining an agent” includes synthesizing, purchasing, or otherwise acquiring one or more agents.

By “programmed cell death 1 polypeptide (PD1)” is meant a protein or a fragment thereof having immune-inhibitory receptor activity and having at least about 85% or greater amino acid sequence identity to NCBI Reference Sequence: NP_005009.2. An exemplary human PD-1 amino acid sequence is provided below:

>NP_005009.2 programmed cell death protein 1
precursor [Homo sapiens]
(SEQ ID NO. 9)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL

By “PD1 polynucleotide” is meant a nucleic acid molecule encoding a PD1 polypeptide. An exemplary sequence is provided at NCBI Ref. No. NM_005018.3, which is reproduced below:

>NM_005018.3 Homo sapiens programmed cell death 1
(PDCD1), mRNA
(SEQ ID NO. 10)
GCTCACCTCCGCCTGAGCAGTGGAGAAGGCGGCACTCTGGTGGGGCTGCT
CCAGGCATGCAGATCCCACAGGCGCCCTGGCCAGTCGTCTGGGCGGTGCT
ACAACTGGGCTGGCGGCCAGGATGGTTCTTAGACTCCCCAGACAGGCCCT
GGAACCCCCCCACCTTCTCCCCAGCCCTGCTCGTGGTGACCGAAGGGGAC
AACGCCACCTTCACCTGCAGCTTCTCCAACACATCGGAGAGCTTCGTGCT
AAACTGGTACCGCATGAGCCCCAGCAACCAGACGGACAAGCTGGCCGCCT
TCCCCGAGGACCGCAGCCAGCCCGGCCAGGACTGCCGCTTCCGTGTCACA
CAACTGCCCAACGGGCGTGACTTCCACATGAGCGTGGTCAGGGCCCGGCG
CAATGACAGCGGCACCTACCTCTGTGGGGCCATCTCCCTGGCCCCCAAGG
CGCAGATCAAAGAGAGCCTGCGGGCAGAGCTCAGGGTGACAGAGAGAAGG
GCAGAAGTGCCCACAGCCCACCCCAGCCCCTCACCCAGGCCAGCCGGCCA
GTTCCAAACCCTGGTGGTTGGTGTCGTGGGCGGCCTGCTGGGCAGCCTGG
TGCTGCTAGTCTGGGTCCTGGCCGTCATCTGCTCCCGGGCCGCACGAGGG
ACAATAGGAGCCAGGCGCACCGGCCAGCCCCTGAAGGAGGACCCCTCAGC
CGTGCCTGTGTTCTCTGTGGACTATGGGGAGCTGGATTTCCAGTGGCGAG
AGAAGACCCCGGAGCCCCCCGTGCCCTGTGTCCCTGAGCAGACGGAGTAT
GCCACCATTGTCTTTCCTAGCGGAATGGGCACCTCATCCCCCGCCCGCAG
GGGCTCAGCTGACGGCCCTCGGAGTGCCCAGCCACTGAGGCCTGAGGATG
GACACTGCTCTTGGCCCCTCTGACCGGCTTCCTTGGCCACCAGTGTTCTG
CAGACCCTCCACCATGAGCCCGGGTCAGCGCATTTCCTCAGGAGAAGCAG
GCAGGGTGCAGGCCATTGCAGGCCGTCCAGGGGCTGAGCTGCCTGGGGGC
GACCGGGGCTCCAGCCTGCACCTGCACCAGGCACAGCCCCACCACAGGAC
TCATGTCTCAATGCCCACAGTGAGCCCAGGCAGCAGGTGTCACCGTCCCC
TACAGGGAGGGCCAGATGCAGTCACTGCTTCAGGTCCTGCCAGCACAGAG
CTGCCTGCGTCCAGCTCCCTGAATCTCTGCTGCTGCTGCTGCTGCTGCTG
CTGCTGCCTGCGGCCCGGGGCTGAAGGCGCCGTGGCCCTGCCTGACGCCC
CGGAGCCTCCTGCCTGAACTTGGGGGCTGGTTGGAGATGGCCTTGGAGCA
GCCAAGGTGCCCCTGGCAGTGGCATCCCGAAACGCCCTGGACGCAGGGCC
CAAGACTGGGCACAGGAGTGGGAGGTACATGGGGCTGGGGACTCCCCAGG
AGTTATCTGCTCCCTGCAGGCCTAGAGAAGTTTCAGGGAAGGTCAGAAGA
GCTCCTGGCTGTGGTGGGCAGGGCAGGAAACCCCTCCACCTTTACACATG
CCCAGGCAGCACCTCAGGCCCTTTGTGGGGCAGGGAAGCTGAGGCAGTAA
GCGGGCAGGCAGAGCTGGAGGCCTTTCAGGCCCAGCCAGCACTCTGGCCT
CCTGCCGCCGCATTCCACCCCAGCCCCTCACACCACTCGGGAGAGGGACA
TCCTACGGTCCCAAGGTCAGGAGGGCAGGGCTGGGGTTGACTCAGGCCCC
TCCCAGCTGTGGCCACCTGGGTGTTGGGAGGGCAGAAGTGCAGGCACCTA
GGGCCCCCCATGTGCCCACCCTGGGAGCTCTCCTTGGAACCCATTCCTGA
AATTATTTAAAGGGGTTGGCCGGGCTCCCACCAGGGCCTGGGTGGGAAGG
TACAGGCGTTCCCCCGGGGCCTAGTACCCCCGCCGTGGCCTATCCACTCC
TCACATCCACACACTGCACCCCCACTCCTGGGGCAGGGCCACCAGCATCC
AGGCGGCCAGCAGGCACCTGAGTGGCTGGGACAAGGGATCCCCCTTCCCT
GTGGTTCTATTATATTATAATTATAATTAAATATGAGAGCATGCTAA.

By “programmed cell death 1 ligand 1 (“PD-L1; PDL1) polypeptide” also known as “CD274 polypeptide”” is meant a protein or a fragment thereof having PD1 binding activity and having at least about 85% or greater amino acid sequence identity to NCBI Reference Sequence: NP_054862.1. An exemplary human PD-L1 amino acid sequence is provided below:

>NP_054862.1 programmed cell death 1 ligand 1
isoform a precursor [Homo sapiens]
(SEQ ID NO. 11)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET

By “PDL1 polynucleotide” is meant a nucleic acid molecule encoding a PDL1 polypeptide. An exemplary sequence is provided at NCBI Ref. No. NM_014143.4, which is reproduced below:

>NM_014143. 4: 70-942 Homo sapiens CD274 molecule
(CD274) , transcript variant 1, mRNA
(SEQ ID NO. 12)
ATGAGGATATTTGCTGTCTTTATATTCATGACCTACTGGCATTTGCTGAA
CGCATTTACTGTCACGGTTCCCAAGGACCTATATGTGGTAGAGTATGGTA
GCAATATGACAATTGAATGCAAATTCCCAGTAGAAAAACAATTAGACCTG
GCTGCACTAATTGTCTATTGGGAAATGGAGGATAAGAACATTATTCAATT
TGTGCATGGAGAGGAAGACCTGAAGGTTCAGCATAGTAGCTACAGACAGA
GGGCCCGGCTGTTGAAGGACCAGCTCTCCCTGGGAAATGCTGCACTTCAG
ATCACAGATGTGAAATTGCAGGATGCAGGGGTGTACCGCTGCATGATCAG
CTATGGTGGTGCCGACTACAAGCGAATTACTGTGAAAGTCAATGCCCCAT
ACAACAAAATCAACCAAAGAATTTTGGTTGTGGATCCAGTCACCTCTGAA
CATGAACTGACATGTCAGGCTGAGGGCTACCCCAAGGCCGAAGTCATCTG
GACAAGCAGTGACCATCAAGTCCTGAGTGGTAAGACCACCACCACCAATT
CCAAGAGAGAGGAGAAGCTTTTCAATGTGACCAGCACACTGAGAATCAAC
ACAACAACTAATGAGATTTTCTACTGCACTTTTAGGAGATTAGATCCTGA
GGAAAACCATACAGCTGAATTGGTCATCCCAGAACTACCTCTGGCACATC
CTCCAAATGAAAGGACTCACTTGGTAATTCTGGGAGCCATCTTATTATGC
CTTGGTGTAGCACTGACATTCATCTTCCGTTTAAGAAAAGGGAGAATGAT
GGATGTGAAAAAATGTGGCATCCAAGATACAAACTCAAAGAAGCAAAGTG
ATACACATTTGGAGGAGACGTAA.

By “PD-1/PD-L1 checkpoint inhibitor” is meant an agent provided herein that the reduces or inhibits the activity or expression of a PD-1 or PD-L1 polypeptide. Exemplary PD-1/PD-L1 checkpoint inhibitors are listed in Table 5. In some embodiments of any of the aspects, the PD-1/PD-L1 checkpoint inhibitor is atezolizumab, avelumab, BMS-936559, MDX-1105, cemiplimab, durvalumab, nivolumab, pembrolizumab, or a pharmaceutically acceptable salt thereof.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects, the disclosure encompasses sequence alterations that result in conservative amino acid substitutions. In some embodiments of any of the aspects, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

“Primer set” means a set of oligonucleotides that may be used, for example, for PCR. A primer set would consist of at least about 2, 4, 6, 8, 10, 12, 14, 16, 18, 20, 30, 40, 50, 60, 80, 100, 200, 250, 300, 400, 500, 600, or more primers.

“Providing a biological subject or sample” means to obtain a biological subject in vivo or in situ, including tissue or cell sample for use in the methods described in the present disclosure. Most often, this will be done by removing a sample of cells from an animal, but also can be accomplished in vivo or in situ or by using previously isolated cells (for example, isolated from another person, at another time, and/or for another purpose).

By “reduces” is meant a negative alteration of at least about 10%, 25%, 50%, 75%, or 100%.

By “reference” or “reference level” is meant a standard or control condition. In embodiments, the reference is the level of an analyte present in a sample obtained from a subject prior to being administered a treatment, obtained from a healthy subject (e.g., a subject not having cancer), or a sample obtained from a subject at an earlier time point than a particular sample time point.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, the reference sequence is the sequence of a reference genome.

A “reference genome” is a defined genome used as a basis for genome comparison or for alignment of sequencing reads thereto. A reference genome may be a subset of or the entirety of a specified genome; for example, a subset of a genome sequence, such as exome sequence, or the complete genome sequence.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene provided herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., or at least about 68° C. In an embodiment, wash steps will occur at 25° C. in 30 mM NaCl, 3 mM trisodium citrate, and 0.1% SDS. In another embodiment, wash steps will occur at 42° C. in 15 mM NaCl, 1.5 mM trisodium citrate, and 0.10% SDS. In another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences provided herein) or nucleic acid sequence (for example, any one of the nucleic acid sequences provided herein). In some embodiments, such a sequence is at least 60%, 80%, 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, primate, or feline. In some embodiments of any of the aspects, the subject has previously been treated with a chemotherapeutic agent. In some embodiments of any of the aspects, the subject has been diagnosed with a drug-resistant lung tumor.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “transforming growth factor, beta-1 (TGF-beta; TGF-β) polypeptide” is meant a protein or a fragment thereof capable of binding a TGF-beta receptor and having at least about 85% or greater amino acid sequence identity to GenBank Accession No. AAH00125.1, provided below. An exemplary human TGF-beta amino acid sequence is provided below:

>AAH00125.1 Transforming growth factor, beta 1
[Homo sapiens]
(SEQ ID NO. 13)
MPPSGLRLLLLLLPLLWLLVLTPGRPAAGLSTCKTIDMELVKRKRIEAIR
GQILSKLRLASPPSQGEVPPGPLPEAVLALYNSTRDRVAGESAEPEPEPE
ADYYAKEVTRVLMVETHNEIYDKFKQSTHSIYMFFNTSELREAVPEPVLL
SRAELRLLRLKLKVEQHVELYQKYSNNSWRYLSNRLLAPSDSPEWLSFDV
TGVVRQWLSRGGEIEGFRLSAHCSCDSRDNTLQVDINGFTTGRRGDLATI
HGMNRPFLLLMATPLERAQHLQSSRHRRALDTNYCFSSTEKNCCVRQLYI
DERKDLGWKWIHEPKGYHANFCLGPCPYIWSLDTQYSKVLALYNQHNPGA

SAAPCCVPQALEPLPIVYYVGRKPKVEQLSNMIVRSCKCS

By “transforming growth factor, beta-1 (TGF-beta; TGF-β) polynucleotide” is meant a nucleic acid molecule or fragment thereof encoding an TGF-beta polypeptide. The sequence of an exemplary TGF-beta polynucleotide is provided at GenBank Accession No. BC000125.1, which is reproduced below.

>BC000125.1: 447-1619 Homo sapiens transforming
growth factor, beta 1, mRNA (cDNA clone MGC:
3119 IMAGE: 3351664) , complete cds
(SEQ ID NO. 14)
ATGCCGCCCTCCGGGCTGCGGCTGCTGCTGCTGCTGCTACCGCTGCTGTG
GCTACTGGTGCTGACGCCTGGCCGGCCGGCCGCGGGACTATCCACCTGCA
AGACTATCGACATGGAGCTGGTGAAGCGGAAGCGCATCGAGGCCATCCGC
GGCCAGATCCTGTCCAAGCTGCGGCTCGCCAGCCCCCCGAGCCAGGGGGA
GGTGCCGCCCGGCCCGCTGCCCGAGGCCGTGCTCGCCCTGTACAACAGCA
CCCGCGACCGGGTGGCCGGGGAGAGTGCAGAACCGGAGCCCGAGCCTGAG
GCCGACTACTACGCCAAGGAGGTCACCCGCGTGCTAATGGTGGAAACCCA
CAACGAAATCTATGACAAGTTCAAGCAGAGTACACACAGCATATATATGT
TCTTCAACACATCAGAGCTCCGAGAAGCGGTACCTGAACCCGTGTTGCTC
TCCCGGGCAGAGCTGCGTCTGCTGAGGCTCAAGTTAAAAGTGGAGCAGCA
CGTGGAGCTGTACCAGAAATACAGCAACAATTCCTGGCGATACCTCAGCA
ACCGGCTGCTGGCACCCAGCGACTCGCCAGAGTGGTTATCTTTTGATGTC
ACCGGAGTTGTGCGGCAGTGGTTGAGCCGTGGAGGGGAAATTGAGGGCTT
TCGCCTTAGCGCCCACTGCTCCTGTGACAGCAGGGATAACACACTGCAAG
TGGACATCAACGGGTTCACTACCGGCCGCCGAGGTGACCTGGCCACCATT
CATGGCATGAACCGGCCTTTCCTGCTTCTCATGGCCACCCCGCTGGAGAG
GGCCCAGCATCTGCAAAGCTCCCGGCACCGCCGAGCCCTGGACACCAACT
ATTGCTTCAGCTCCACGGAGAAGAACTGCTGCGTGCGGCAGCTGTACATT
GACTTCCGCAAGGACCTCGGCTGGAAGTGGATCCACGAGCCCAAGGGCTA
CCATGCCAACTTCTGCCTCGGGCCCTGCCCCTACATTTGGAGCCTGGACA
CGCAGTACAGCAAGGTCCTGGCCCTGTACAACCAGCATAACCCGGGCGCC
TCGGCGGCGCCGTGCTGCGTGCCGCAGGCGCTGGAGCCGCTGCCCATCGT
GTACTACGTGGGCCGCAAGCCCAAGGTGGAGCAGCTGTCCAACATGATCG
TGCGCTCCTGCAAGTGCAGCTGA

By “TGF-beta inhibitor” is meant an agent that reduces the activity or expression of a TGF-beta polypeptide. Non-limiting examples of TGF-beta inhibitors include Galunisertib, Vactosertib, Trabedersen, ISTH0036, Fresolimumab, Disitertide, Lucanix™, Gemogenovatucel-T, and pharmaceutically acceptable salts thereof.

As used herein, the terms “treat,” treating,” “treatment,” and the like refer to reducing or ameliorating a disorder (e.g., lung cancer) and/or symptoms associated therewith. It will be appreciated that, although not precluded, treating a disorder or condition does not require that the disorder, condition or symptoms associated therewith be completely eliminated.

A tumor “responds” to a particular agent provided herein if tumor progression is inhibited as defined above.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1D provide a schematic, a confusion matrix, a heat map, and a chart showing study design and mutational landscape of lung adenocarcinoma (LUAD) expression subtypes. FIG. 1A provides a schematic representation of study design for the identification of TCGA lung adenocarcinoma (LUAD) expression subtypes. 5 lung adenocarcinoma (LUAD) expression subtypes were identified by SignatureAnalyzer. The upper heatmap shows the values of the normalized H matrix identified by the SignatureAnalyzer (row: five expression subtypes, column: 509 TCGA lung adenocarcinoma (LUAD) samples). Samples with normalized association scores higher than 0.6 to a certain subtype were assigned to the subtype. The patient size for each subtype ranges from 7.3% (least common subtype) to 35% (most common subtype) of all cases. The lower heatmap shows the row z-scores of mRNA expression of 100 subtype marker genes for each subtype. Expression subtypes for Cancer Cell Line Encyclopedia (CCLE) lung adenocarcinoma (LUAD) samples (n=78) and Clinical Proteomic Tumor Analysis Consortium (CPTAC) lung adenocarcinoma (LUAD) samples (n=112) were determined by projecting lung adenocarcinoma (LUAD) expression subtypes to each tumor sample using subtype marker gene expression. Tumor samples were assigned to certain expression subtypes based on normalized association values. (cutoff of 0.6). FIG. 1B provides a confusion matrix showing concordance between the lung adenocarcinoma (LUAD) expression subtypes and TCGA lung adenocarcinoma (LUAD) expression subtypes (or Cluster of Clusters Analysis (COCA) expression subtypes). Lung adenocarcinoma (LUAD) expression subtypes were represented in Cancer Cell Line Encyclopedia (CCLE) and Clinical Proteomic Tumor Analysis Consortium (CPTAC) lung adenocarcinoma (LUAD) samples. The cell count in the middle shows the number of samples overlapping between two subtypes. The column-wise proportion is shown at the bottom of each cell and the row-wise proportion is shown on the right side of each cell. The bar plots at the bottom show the column-wise proportion and the bar plots on the right side of the heatmap show the row-wise proportion. FIG. 1C provides a heatmap that shows overall pathway activation profiles (in row z-scores of GSVA enrichment scores for MSigDB hallmark gene sets) in TCGA lung adenocarcinoma (LUAD) expression subtypes. FIG. 1D provides a chart showing driver mutations identified by MutSig2CV (point mutations, indels; Q value <0.01) for each TCGA lung adenocarcinoma (LUAD) expression subtype.

FIGS. 2A and 2B provide a heat map and boxplots showing subtype-specific cancer vulnerabilities. FIG. 2A provides a heatmap showing the values of the normalized H matrix. The heatmap shows subtypes represented in Cancer Cell Line Encyclopedia (CCLE) lung adenocarcinoma (LUAD) (mostly subtypes 3 and 4). Samples with normalized association scores higher than 0.5 to a certain subtype and difference between the highest association score and the second highest association score higher than 0.2 were assigned to the subtype. Subtype 3 (n=31) and 4 (n=16) cell lines were represented in the Cancer Cell Line Encyclopedia (CCLE) lung adenocarcinoma (LUAD) dataset. FIG. 2B provides boxplots showing CERES scores of CDK6 and CCND3 in S4 versus other cell lines. CDK6 and CCND3 were Subtype 4-specific vulnerabilities. Lung adenocarcinoma (LUAD) driver oncogenes (genes with recurrent point mutations, indels, and somatic copy-number alterations (SCNAs) identified from this study) identified from this study (n=21) were tested. Top genes with subtype-specific cancer vulnerabilities were selected as the gene with median CERES score in S4 was lower than −0.5 and median difference in CERES scores between S4 and others less than −0.2. The common essential genes (Achilles common essential genes) were filtered out from the top gene list. P values were calculated by the Wilcoxon rank sum test. The one sample assigned to S1 is not shown due to the small sample size of S1.

FIGS. 3A-3C present heat maps and bar graphs showing results from a proteogenomic analysis of genes with subtype-specific recurrent somatic copy-number alterations (SCNAs). FIG. 3A presents heatmaps, where the upper heatmap shows the normalized H matrix for Clinical Proteomic Tumor Analysis Consortium (CPTAC) lung adenocarcinoma (LUAD) samples (row: five lung adenocarcinoma (LUAD) expression subtypes, column: Clinical Proteomic Tumor Analysis Consortium (CPTAC) lung adenocarcinoma (LUAD) samples). Clinical Proteomic Tumor Analysis Consortium (CPTAC) lung adenocarcinoma (LUAD) tumor samples were assigned to certain lung adenocarcinoma (LUAD) expression subtypes based on normalized association values (cutoff of 0.6). The column annotation below shows the assigned expression subtypes. The middle heatmap shows the row z-scores of GSVA enrichment scores of MSigDB hallmark gene sets among Clinical Proteomic Tumor Analysis Consortium (CPTAC) lung adenocarcinoma (LUAD) samples. Pathway activation consistent with lung adenocarcinoma (LUAD) expression subtypes are highlighted with the black box. The lower heatmap shows the row z-scores of S3/S4/S5 marker gene expression across Clinical Proteomic Tumor Analysis Consortium (CPTAC) lung adenocarcinoma (LUAD) samples. FIG. 3B provides barplots and a heatmap. The barplots show the proportion of The Cancer Genome Atlas (TCGA)/Clinical Proteomic Tumor Analysis Consortium (CPTAC) lung adenocarcinoma (LUAD) samples in S3/S4/S5 with gene amplification or deletion for selected genes which had recurrent somatic copy-number alterations (SCNAs) in at least one of the S3/S4/S5 subtypes. The heatmap shows the cosine similarity among S3/S4/S5 tumors in TCGA and CPTAC data. FIG. 3C provides boxplots showing protein abundance of genes with recurrent SCNAs across CPTAC lung adenocarcinoma (LUAD) expression subtypes. Copy number states of genes are shown with different shades of grey (medium-grey: amplification, darker grey: deletion, lightest shade of grey: no SCNAs).

FIGS. 4A-4E provide boxplots and scatterplots showing MET as a core regulator of proliferation and PD-L1 expression in subtype 3. FIG. 4A provides boxplots showing copy number, mRNA expression, protein abundance, and phosphorylation of CD274 gene across lung adenocarcinoma (LUAD) expression subtypes. PD-L1 copy number and expression (mRNA, protein, phosphorylation) were significantly higher in subtype 3 vs. subtype 4. FIG. 4B provides boxplots showing copy number, mRNA expression, protein abundance, and phosphorylation of MET gene across lung adenocarcinoma (LUAD) expression subtypes. MET copy number and expression (mRNA, protein, phosphorylation) was significantly higher in subtype 3 vs. 4. FIG. 4C provides scatter plots showing correlation between MET gene expression and gene expression of cytolytic markers (GZMB, GZMA, and PRF1) in S3 versus S4. Not intending to be bound by theory, regulation of PD-L1 expression by MET was stronger in Subtype 3 vs. Subtype 4. FIG. 4D provides boxplots showing proliferation scores and lymphocyte infiltration signature scores (obtained from Thorsson et al., 2018) across lung adenocarcinoma (LUAD) expression subtypes. Both Subtype 3 and Subtype 4 showed high proliferation scores, but Subtype 3 showed higher immune scores than Subtype 4. FIG. 4E provides boxplots showing protein abundance and phosphorylation level of genes in immune-related pathways among CPTAC lung adenocarcinoma (LUAD) S3, S4, and S5. Proteomic and phosphorylation data showed increased immune activity in interferon gamma specific to subtype 3.

FIGS. 5A-5E provide boxplots, immunofluorescence staining images, scatter plots, and a schematic showing c-MET inhibition drives PD-L1 expression in cell lines. FIG. 5A provides boxplots showing response to Tivantinib measured by the delta change in confluency between treatment and untreated (DMSO only) cell lines within each subtype. FIG. 5B provides images of immunofluorescence staining under Tivantinib treatment. Anti-PD-L1 antibody staining is shown in the first column on the left; DAPI for nuclear staining is shown in the middle column; overlay of both stainings is shown in the left column. FIG. 5C provides boxplots showing fluorescent images quantification using ImageJ software after background correction. FIG. 5D provides scatter plots showing correlation between MET gene expression and gene expression of GSK3β in Cancer Cell Line Encyclopedia (CCLE) data in the different subtypes. Not intending to be bound by theory, FIG. 5E provides a schematic diagram showing MET is a core regulator of proliferation and PD-L1 expression regulation through the GSK3β axis in subtype 3 tumors.

FIG. 6 provides a chart showing results from biomarker discovery for lung adenocarcinoma (LUAD) expression subtypes. The chart summarizes biomarkers of lung adenocarcinoma (LUAD) expression subtypes S3 and S4 based on gene expression data and reverse-phase protein array (RPPA) data. The representative prediction model represents the model with the lambda that minimizes the cross-validation prediction error rate. The 5-feature model represents the model with the only five features included for parsimony of the model and clinical utility.

FIG. 7 presents a chart showing subtype-specific proteogenomic features and potential therapeutic targets for subtypes. The chart summarizes subtype-specific proteogenomic features identified and potential therapeutic targets for subtypes.

FIGS. 8A-8F provide confusion matrices, heatmaps, and Kaplan-Meier curves showing identification and characterization of 5 newly identified lung adenocarcinoma (LUAD) expression subtypes. FIGS. 8A-8C provide confusion matrices showing concordance between two groups of subtypes of interest. The cell count in the middle shows the number of samples overlapping between two subtypes. The column-wise proportion is shown at the bottom of each cell and the row-wise proportion is shown on the right side of each cell. FIG. 8D provides a heatmap showing overall pathway activation profiles (in row z-scores of GSVA enrichment scores for MSigDB hallmark gene sets) of tumors with PI subtype mapped to S1, S2 or S3. FIG. 8E provides Kaplan-Meier curves for the disease-specific survival (DSS) among TCGA lung adenocarcinoma (LUAD) expression subtypes. The P value was calculated by the log rank test. FIG. 8F provides a heatmap showing row z-scores of mRNA expression of subtype marker genes across 5 expression subtypes.

FIGS. 9A-9B provides co-mutation plots by subtypes (MutSig2CV), and GISTIC plots by subtypes. FIG. 9A provides co-mutation plots. MutSig2CV was run for each expression subtype for subtype-specific driver mutation discovery. The resulting co-mutation plots show the driver mutations for each subtype (Subtypes 1, 2, 3, 4, and 5) based on the Q value cutoff of 0.1. FIG. 9B shows GISTIC plots. GISTIC2.0 was run for each expression subtype for subtype-specific recurrent somatic copy number alterations (SCNA) analysis (left panel: deletion; right panel: amplification; Q value <0.1). In the “Subtype 1” co-mutation plot of FIG. 9A, the following identifiers are listed from left-to-right on the lower axis: TCGA-86-A4JF; TCGA-95-7039; TCGA-95-7567; TCGA-69-7980; TCGA-44-8117; TCGA-99-8025; TCGA-55-8616; TCGA-55-8514; TCGA-55-8506; TCGA-L9-A7SV; TCGA-86-8073; TCGA-69-7979; TCGA-78-8662; TCGA-49-AARN; TCGA-50-5946; TCGA-44-8120; TCGA-62-8399; TCGA-86-8279; TCGA-97-7937; TCGA-MN-A4N1; TCGA-55-A491; TCGA-78-7147; TCGA-L4-A4E5; TCGA-78-7535; TCGA-44-A4SU; TCGA-62-8394; TCGA-93-A4JN; TCGA-49-AAQV; TCGA-55-A48Z; TCGA-55-1595; TCGA-L9-A443; TCGA-73-4668; TCGA-95-7948; TCGA-73-4675; TCGA-71-6725; and TCGA-44-A47B. In the “Subtype 2” co-mutation plot of FIG. 9A, the following identifiers are listed from left-to-right on the lower axis: TCGA-05-4382; TCGA-69-7765; TCGA-55-8096; TCGA-05-4402; TCGA-38-6178; TCGA-69-7760; TCGA-86-8074; TCGA-97-8547; TCGA-49-4490; TCGA-86-8055; TCGA-86-8075; TCGA-55-6980; TCGA-97-7554; TCGA-55-7576; TCGA-49-4505; TCGA-64-1679; TCGA-55-6985; TCGA-05-4405; TCGA-44-6777; TCGA-50-6593; TCGA-44-6774; TCGA-05-4430; TCGA-44-6775; TCGA-38-4628; TCGA-J2-8192; TCGA-55-6981; TCGA-MP-A4T9; TCGA-38-4627; TCGA-55-7281; TCGA-64-5815; TCGA-MP-A4SY; TCGA-91-6829; TCGA-44-4112; TCGA-73-4658; TCGA-55-6982; TCGA-44-3398; TCGA-05-5715; TCGA-86-8278; TCGA-62-A46V; TCGA-86-6562; TCGA-44-2665; TCGA-49-4512; and TCGA-55-8091. In the “Subtype 3” co-mutation plot of FIG. 9A, the following identifiers are listed from left-to-right on the lower axis: TCGA-MP-A4T4; TCGA-78-7145; TCGA-69-7978; TCGA-55-7907; TCGA-55-8302; TCGA-MP-A4TK; TCGA-69-7974; TCGA-64-5775; TCGA-62-A46R; TCGA-05-4427; TCGA-64-5778; TCGA-99-8028; TCGA-91-6836; TCGA-MN-A4N5; TCGA-44-7661; TCGA-MP-A4TI; TCGA-83-5908; TCGA-55-7911; TCGA-55-7726; TCGA-93-A4JO; TCGA-55-8511; TCGA-55-6712; TCGA-50-5933; TCGA-44-A47G; TCGA-55-7903; TCGA-78-7146; TCGA-49-AAR3; TCGA-73-4676; TCGA-44-3918; TCGA-L9-A444; TCGA-49-6767; TCGA-50-5045; TCGA-55-8089; TCGA-55-7994; TCGA-55-8205; TCGA-L9-A743; TCGA-91-6848; TCGA-73-4666; TCGA-55-8510; TCGA-05-4410; TCGA-50-6594; TCGA-05-5428; TCGA-78-8660; TCGA-44-2662; TCGA-55-6979; TCGA-62-A472; TCGA-38-4632; TCGA-50-5049; TCGA-64-1676; TCGA-50-5066; TCGA-69-A59K; TCGA-55-8208; TCGA-55-7574; TCGA-97-A4LX; TCGA-44-7662; TCGA-MP-A4SV; TCGA-86-6851; TCGA-L9-A8F4; TCGA-64-1677; TCGA-MN-A4N4; TCGA-50-6590; TCGA-44-2656; TCGA-05-4398; TCGA-49-AAR4; TCGA-49-4487; TCGA-44-2668; TCGA-35-4122; TCGA-35-4123; TCGA-55-8301; TCGA-55-A493; TCGA-MP-A4TC; TCGA-44-3396; TCGA-75-5125; TCGA-95-8494; TCGA-50-6595; TCGA-75-6207; TCGA-62-A46U; TCGA-05-4426; TCGA-97-8175; TCGA-50-5941; TCGA-49-AARO; TCGA-75-5126; TCGA-55-A490; TCGA-05-4244; TCGA-05-4250; TCGA-44-7672; TCGA-95-A4VN; TCGA-55-8299; TCGA-49-6745; TCGA-49-6761; TCGA-75-5122; TCGA-75-6205; TCGA-55-6978; TCGA-49-4488; TCGA-55-6971; TCGA-NJ-A4YQ; TCGA-55-6987; TCGA-50-5044; TCGA-38-4625; TCGA-49-4494; TCGA-93-A4JQ; TCGA-05-4434; TCGA-86-8671; TCGA-50-5055; and TCGA-78-8648. In the “Subtype 4” co-mutation plot of FIG. 9A, the following identifiers are listed from left-to-right on the lower axis: TCGA-73-4670; TCGA-05-4395; TCGA-4B-A93V; TCGA-55-8094; TCGA-99-8033; TCGA-49-AAR0; TCGA-44-6779; TCGA-50-5939; TCGA-53-7624; TCGA-49-AARE; TCGA-35-5375; TCGA-62-A46O; TCGA-49-AAR9; TCGA-67-3771; TCGA-55-A4DF; TCGA-86-8358; TCGA-75-6211; TCGA-95-7947; TCGA-44-7660; TCGA-95-7043; TCGA-55-7995; TCGA-53-A4EZ; TCGA-55-8620; TCGA-44-A4SS; TCGA-44-7667; TCGA-55-5899; TCGA-44-6778; TCGA-49-4514; TCGA-86-8672; TCGA-55-8085; TCGA-78-7154; TCGA-55-6975; TCGA-49-AARQ; TCGA-MP-A4TF; TCGA-44-6145; TCGA-95-7562; TCGA-93-8067; TCGA-55-7910; TCGA-44-A479; TCGA-55-1596; TCGA-78-7220; TCGA-05-5425; TCGA-75-7031; TCGA-55-8092; TCGA-55-8507; TCGA-NJ-A4YF; TCGA-50-5930; TCGA-78-8640; TCGA-05-4432; TCGA-86-8585; TCGA-55-6969; TCGA-MP-A4TA; TCGA-J2-A4AD; TCGA-86-7711; TCGA-55-6968; TCGA-91-A4BC; TCGA-50-6591; TCGA-44-7670; TCGA-86-8673; TCGA-86-7701; TCGA-05-4397; TCGA-91-6847; TCGA-78-7155; TCGA-44-5644; TCGA-75-6214; TCGA-78-7536; TCGA-78-7150; TCGA-78-7542; TCGA-55-7570; TCGA-50-5931; TCGA-91-8499; TCGA-05-4420; TCGA-L9-A5IP; TCGA-50-6592; TCGA-91-6831; TCGA-95-7944; TCGA-55-8614; TCGA-86-7954; TCGA-55-8204; TCGA-55-1594; TCGA-62-A471; TCGA-91-6840; TCGA-73-7499; TCGA-62-8402; TCGA-75-5147; TCGA-NJ-A55R; TCGA-78-7166; TCGA-MP-A4TE; TCGA-97-8176; TCGA-50-5936; TCGA-55-7815; TCGA-62-8398; TCGA-05-4418; TCGA-75-7027; TCGA-49-4506; TCGA-50-5051; TCGA-49-4507; TCGA-44-7669; TCGA-38-4631; TCGA-49-6742; TCGA-64-5781; TCGA-49-6743; TCGA-49-AAR2; TCGA-05-4389; TCGA-55-8505; TCGA-86-8054; TCGA-NJ-A4YP; TCGA-05-4417; TCGA-55-8508; TCGA-80-5608; TCGA-95-A4VP; TCGA-86-7713; TCGA-05-4415; TCGA-55-8203; TCGA-99-8032; TCGA-78-7161; TCGA-86-8359; TCGA-55-8615; TCGA-MP-A4TD; TCGA-05-4390; TCGA-69-7973; TCGA-53-7813; TCGA-64-5774; TCGA-55-6642; TCGA-91-6828; TCGA-55-A494; TCGA-MP-A4T8; TCGA-86-7953; TCGA-73-A9RS; TCGA-44-8119; TCGA-50-5072; TCGA-78-7159; TCGA-73-4659; TCGA-55-7913; TCGA-69-8255; TCGA-55-A48Y; TCGA-80-5611; TCGA-64-5779; TCGA-86-7955; TCGA-50-7109; TCGA-86-8669; TCGA-44-5643; and TCGA-05-4422. In the “Subtype 5” co-mutation plot of FIG. 9A, the following identifiers are listed from left-to-right on the lower axis: TCGA-97-8179; TCGA-97-7938; TCGA-86-8056; TCGA-55-7283; TCGA-55-7914; TCGA-05-4433; TCGA-78-7540; TCGA-73-7498; TCGA-35-3615; TCGA-44-7671; TCGA-44-6776; TCGA-55-6970; TCGA-73-4677; TCGA-J2-8194; TCGA-78-7167; TCGA-86-8674; TCGA-69-8253; TCGA-MP-A4T7; TCGA-91-6849; TCGA-75-6206; TCGA-78-7148; TCGA-44-A47A; TCGA-69-8254; TCGA-55-6983; TCGA-55-8090; TCGA-78-7160; TCGA-NJ-A550; TCGA-62-A46S; TCGA-67-3774; TCGA-67-4679; TCGA-97-7941; TCGA-78-8655; TCGA-05-4249; TCGA-78-7539; TCGA-NJ-A4YI; TCGA-J2-A4AG; TCGA-50-5932; TCGA-97-A4M0; TCGA-86-A456; TCGA-55-8207; TCGA-95-A4VK; TCGA-73-4662; TCGA-97-A4M5; TCGA-NJ-A4YG; TCGA-55-7725; TCGA-93-7347; TCGA-67-3773; TCGA-55-7728; TCGA-50-8459; TCGA-55-8097; TCGA-86-8076; TCGA-55-7284; TCGA-49-4510; TCGA-44-6146; TCGA-55-8512; TCGA-55-6984; TCGA-75-7030; TCGA-05-4384; TCGA-78-7149; TCGA-55-7727; TCGA-05-4424; TCGA-86-8280; TCGA-75-5146; TCGA-50-5944; TCGA-05-5423; TCGA-64-1681; TCGA-93-A4JP; TCGA-86-A4P7; TCGA-55-7573; TCGA-50-6673; TCGA-97-A4M1; TCGA-44-5645; TCGA-55-A57B; TCGA-50-8460; TCGA-55-1592; TCGA-55-A48X; TCGA-55-A4DG; TCGA-99-7458; TCGA-44-2659; TCGA-53-7626; TCGA-05-4396; TCGA-01-A52J; TCGA-95-8039; TCGA-50-5068; TCGA-97-7553; TCGA-62-A46Y; TCGA-78-7158; TCGA-62-A470; TCGA-55-A492; TCGA-78-7156; TCGA-97-A4M3; TCGA-78-7153; TCGA-78-7633; TCGA-MP-A5C7; TCGA-55-6972; TCGA-86-8281; TCGA-44-2655; TCGA-49-4486; TCGA-78-7162; TCGA-97-A4M7; TCGA-L9-A50 W; TCGA-MP-A4SW; TCGA-78-7537; TCGA-05-4403; TCGA-44-7659; TCGA-62-A46P; TCGA-91-7771; TCGA-78-7152; TCGA-97-8171; TCGA-97-A4M6; TCGA-75-7025; TCGA-75-6212; TCGA-67-6217; TCGA-97-8172; TCGA-91-6835; TCGA-MP-A4T6; TCGA-67-3772; TCGA-49-4501; TCGA-97-8177; TCGA-86-8668; TCGA-55-8206; TCGA-97-8552; TCGA-05-4425; TCGA-49-6744; TCGA-44-2666; TCGA-80-5607; TCGA-86-7714; TCGA-50-5942; TCGA-67-6216; TCGA-55-6986; TCGA-64-1680; TCGA-91-A4BD; TCGA-L4-A4E6; TCGA-44-2657; TCGA-93-7348; TCGA-50-6597; TCGA-J2-A4AE; TCGA-38-7271; TCGA-62-8395; TCGA-50-8457; TCGA-97-7546; TCGA-97-7547; TCGA-38-4626; TCGA-91-6830; TCGA-55-7724; TCGA-97-8174; TCGA-S2-AA1A; TCGA-NJ-A55A; TCGA-MP-A4TH; TCGA-67-6215; TCGA-50-5935; TCGA-69-7764; TCGA-MP-A4TJ; TCGA-69-7763; TCGA-55-8621; TCGA-99-AA5R; TCGA-44-3919; TCGA-91-8497; TCGA-97-7552; TCGA-NJ-A7XG; TCGA-05-5429; TCGA-91-8496; TCGA-55-8087; TCGA-69-7761; TCGA-97-A4M2; TCGA-62-8397; TCGA-78-7163; TCGA-55-8619; TCGA-55-6543; TCGA-75-6203; TCGA-38-A44F; TCGA-44-6148; TCGA-55-8513; TCGA-49-AARR; and TCGA-86-A4P8.

FIGS. 10A-10J provide boxplots showing additional subtype-specific genomic feature data. The boxplots show the scores of genomic features obtained from Thorsson, Vésteinn, et al. “The immune landscape of cancer.” Immunity 48.4 (2018): 812-830 among lung adenocarcinoma (LUAD) expression subtypes.

FIGS. 11A and 11B provide a heat map and boxplots showing additional subtype-specific immune cell subset fraction data obtained from Thorsson, Vésteinn, et al. “The immune landscape of cancer.” Immunity 48.4 (2018): 812-830. FIG. 11A provides a heatmap showing immune cell subset fraction (in row z-scores of immune cell subset fraction values of TCGA lung adenocarcinoma (LUAD) tumors across expression subtypes. FIG. 11B provides boxplots showing the selected immune cell subset fraction among lung adenocarcinoma (LUAD) expression subtypes.

FIGS. 12A-12C provide bar graphs and boxplots showing concordance of frequencies in recurrent genomic alterations between TCGA and Cancer Cell Line Encyclopedia (CCLE) lung adenocarcinoma (LUAD) expression subtypes. FIG. 12A provides bar graphs showing the percentage of TCGA/Cancer Cell Line Encyclopedia (CCLE) lung adenocarcinoma (LUAD) samples with recurrent SNVs/Indels observed in the TCGA lung adenocarcinoma (LUAD) cohort. The Bayesian credible interval was used for a 95% credible interval. Cancer Cell Line Encyclopedia (CCLE) lung adenocarcinoma (LUAD) subtypes harbored similar recurrent somatic point mutations and indels as TCGA lung adenocarcinoma (LUAD) subtypes. FIG. 12B provides bar graphs showing the percentage of TCGA/Cancer Cell Line Encyclopedia (CCLE) lung adenocarcinoma (LUAD) samples with recurrent copy number alterations observed in the TCGA lung adenocarcinoma (LUAD) cohort. The Bayesian credible interval was used for a 95% credible interval. Gene amplification in TCGA lung adenocarcinoma (LUAD) was based on the entries having values of +2 (high-level threshold) or +1 (low-level threshold) in the ‘all_thresholded.by_genes.txt’ from the GISTIC 2.0. Gene deletion in TCGA lung adenocarcinoma (LUAD) was based on the entries having values of −2 (high-level threshold) or −1 (low-level threshold) in the ‘all_thresholded.by_genes.txt’ from the GISTIC 2.0. Gene amplification and deletion in Cancer Cell Line Encyclopedia (CCLE) lung adenocarcinoma (LUAD) was based on a log 2 copy number ratio threshold of 0.3. Cancer Cell Line Encyclopedia (CCLE) lung adenocarcinoma (LUAD) subtypes harbored similar recurrent copy-number alterations (CNAs) as TCGA lung adenocarcinoma (LUAD) subtypes. FIG. 12C provides boxplots showing response to CDK4/6 inhibitors measured by the delta change in confluency between treatment and untreated (DMSO only) cell lines within each subtype. Left panel—response to Palbociclib (CDK4 specific concentration—11 nM), middle panel—response to CDK4/6 Inhibitor IV (CDK4 specific concentration—1.5 μM) and right panel—response to Palbociclib (CDK4/6 concentration—16 nM).

FIGS. 13A-13D provides a heatmap, pie charts, and boxplots showing Clinical Proteomic Tumor Analysis Consortium (CPTAC) lung adenocarcinoma (LUAD) expression subtypes and protein expression of genes with recurrent somatic copy-number alterations (SCNAs) in The Cancer Genome Atlas (TCGA) lung adenocarcinoma (LUAD). FIG. 13A provides a heatmap showing row z-scores of mRNA expression of 5,000 most variable genes across Clinical Proteomic Tumor Analysis Consortium (CPTAC) lung adenocarcinoma (LUAD) samples. The upper column annotations show the newly-identified TCGA expression subtypes (upper), CPTAC multi-omics clusters (middle), and the original TCGA lung adenocarcinoma (LUAD) expression subtypes (lower) of CPTAC lung adenocarcinoma (LUAD) samples. Recurrent CNAs in TCGA lung adenocarcinoma (LUAD) showed similar proportion of samples with CNAs in CPTAC lung adenocarcinoma (LUAD) Subtype 5 (n=58), but not in CPTAC lung adenocarcinoma (LUAD) Subtypes 3 and 4. Not intending to be bound by theory, this may be due to smaller sample size in CPTAC lung adenocarcinoma (LUAD) Subtypes 3 (n=13) and 4 (n=13) tumors. FIG. 13B provides pie charts showing ethnicity distribution across all, S3, S4, or S5 tumors in TCGA lung adenocarcinoma (LUAD) cohort versus CPTAC lung adenocarcinoma (LUAD) cohort. FIG. 13C provides boxplots showing protein abundance of genes with recurrent SCNAs in tumors versus normal adjacent tissues among CPTAC lung adenocarcinoma (LUAD) expression subtypes. FIG. 13D presents boxplots showing MET protein expression in NAT versus tumors in S3 and S4.

FIGS. 14A and 14B provide boxplots showing measured mRNA expression levels of the indicated genes associated with the indicated subtypes.

FIGS. 15A-15C provide boxplots. FIG. 15A shows supporting proteomic evidence for MET pathway activation (GAB1, BCL2L1). FIG. 15B shows supporting proteomic evidence for cell proliferation (MCM, LMNA). FIG. 15C shows supporting proteomic evidence for immune pathway activation (antigen presentation, interferon signaling).

### DETAILED DESCRIPTION OF THE INVENTION

As described below, the disclosure provided herein features molecular classifiers and a targeted gene expression panel for use in the characterization of lung cancer (e.g., lung adenocarcinoma) and provides methods of diagnosing, selecting, and treating a subject with cancer with a targeted cancer therapeutic agent.

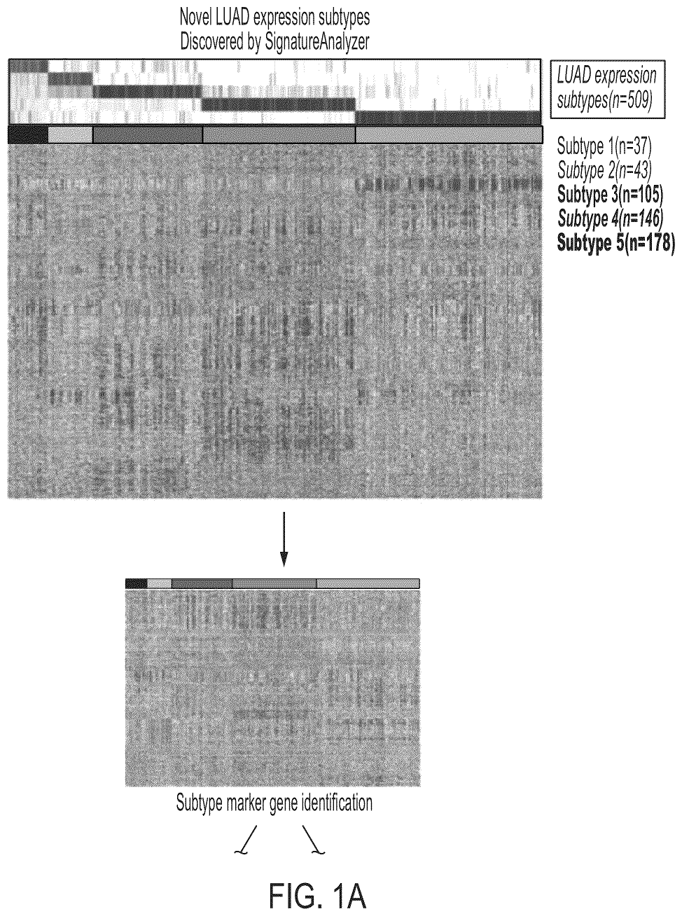

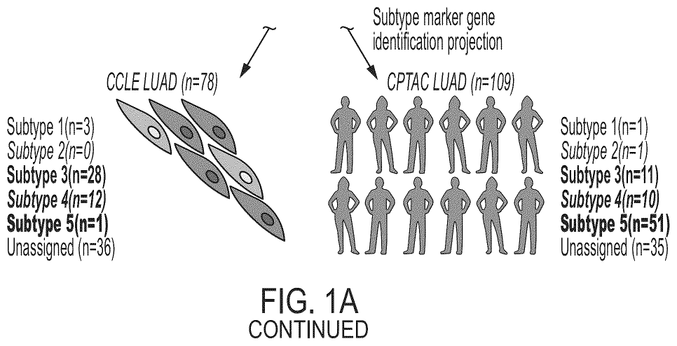

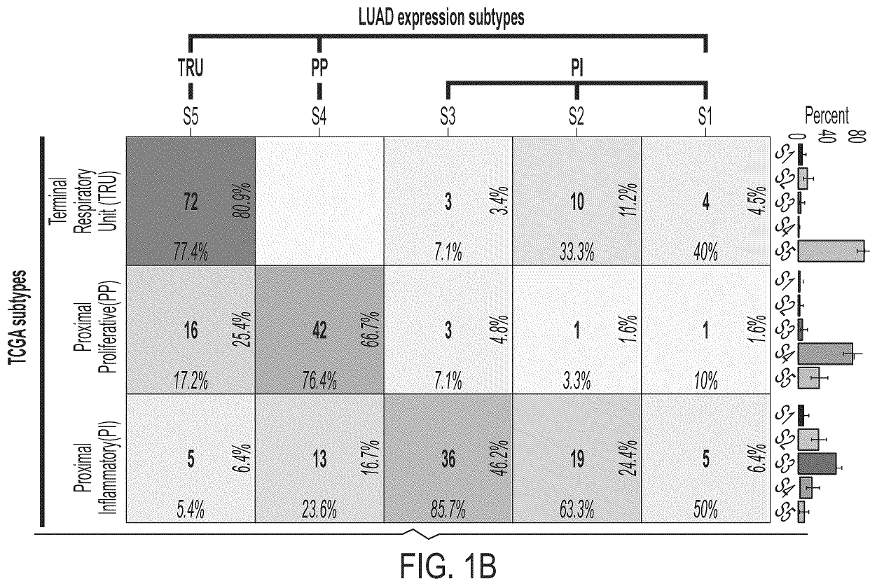

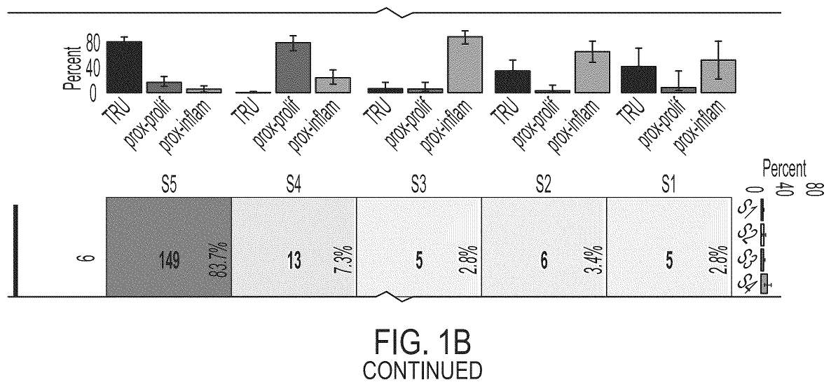

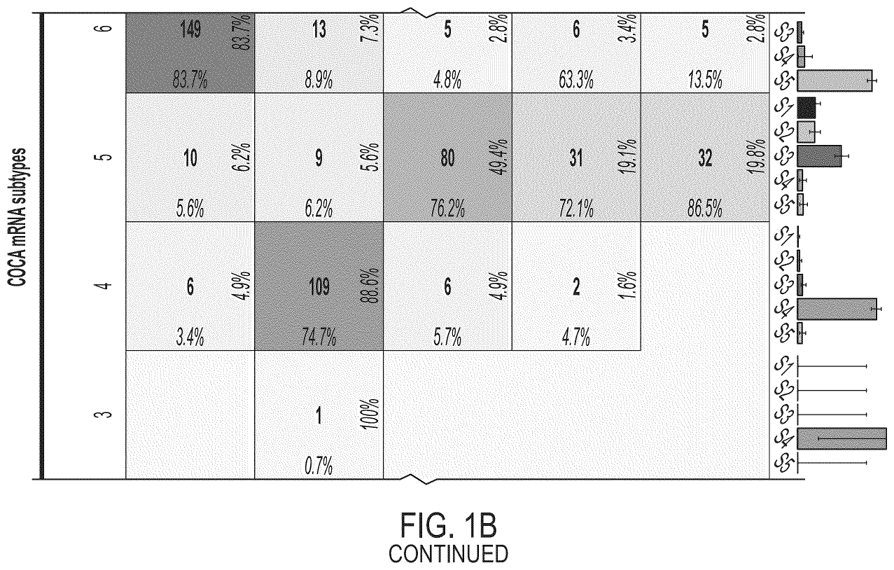

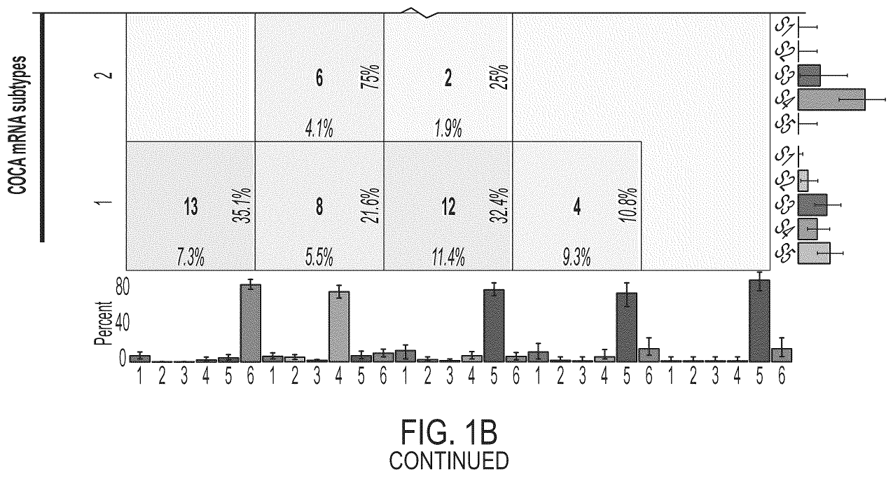

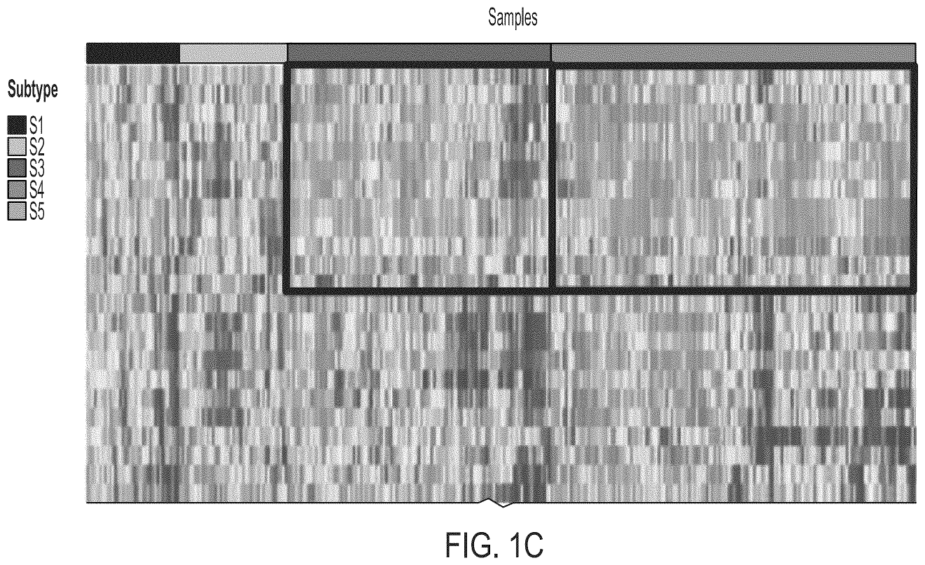

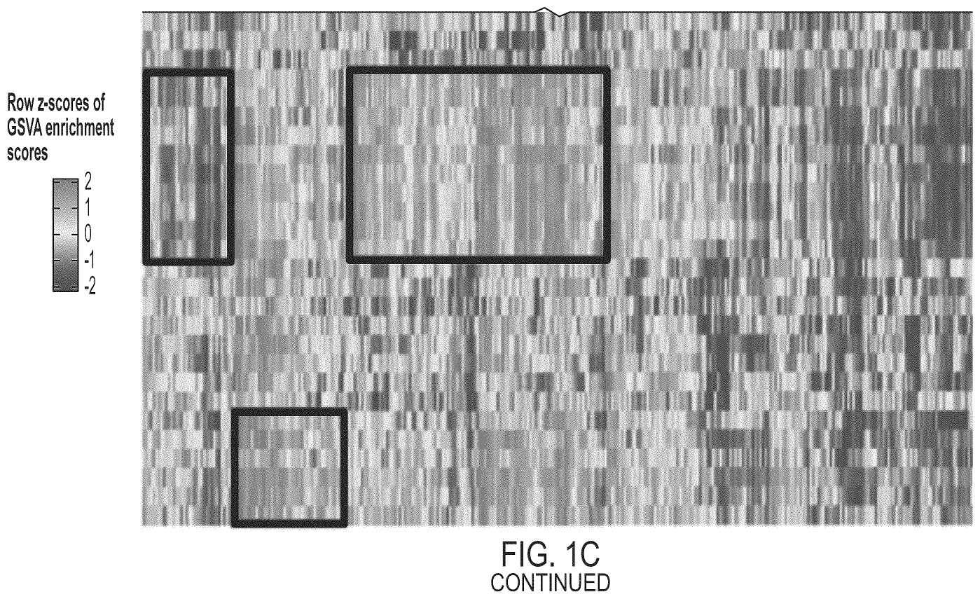

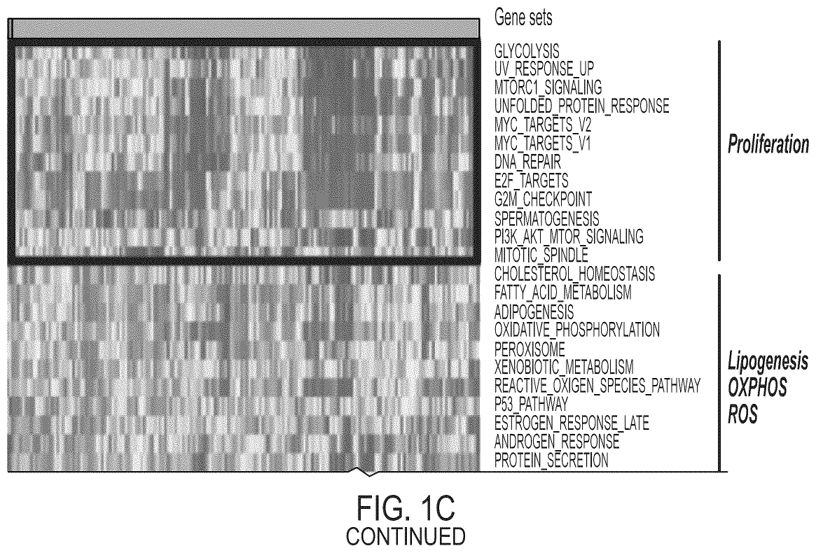

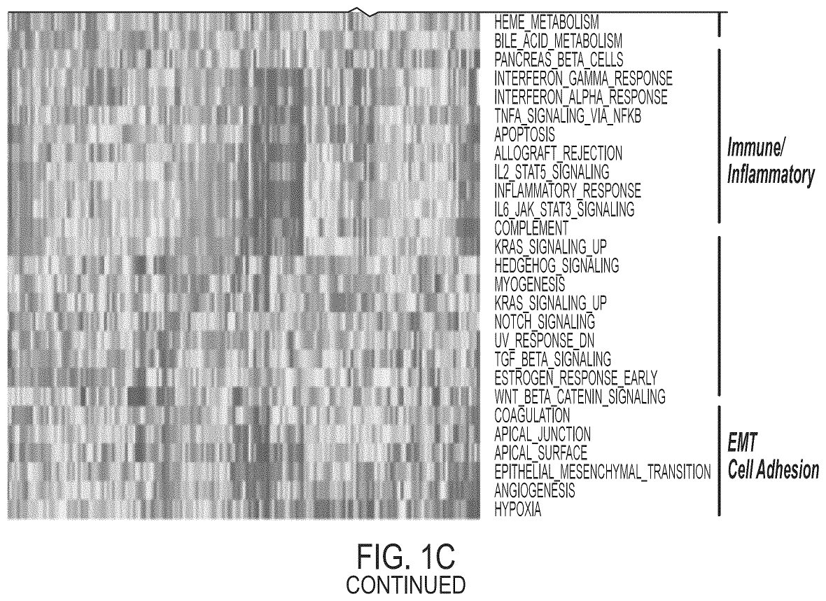

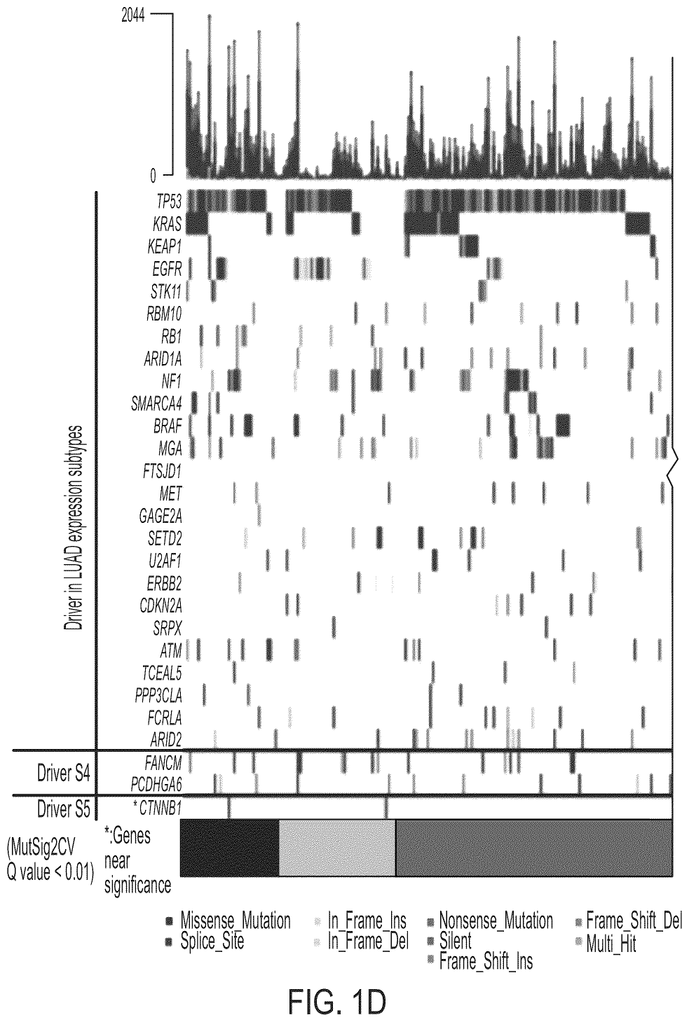

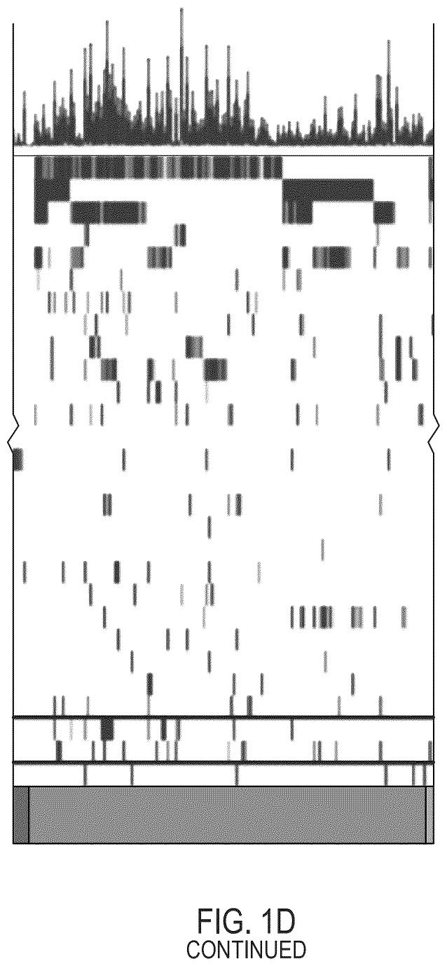

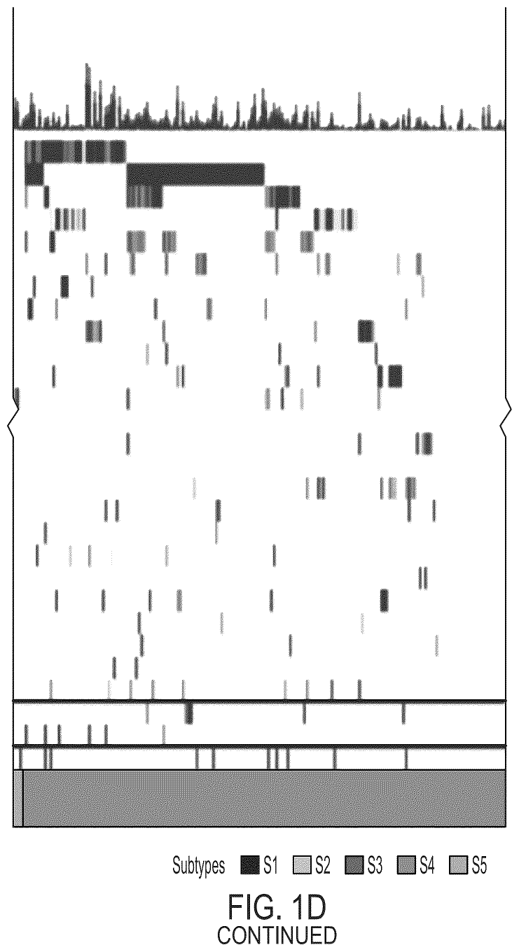

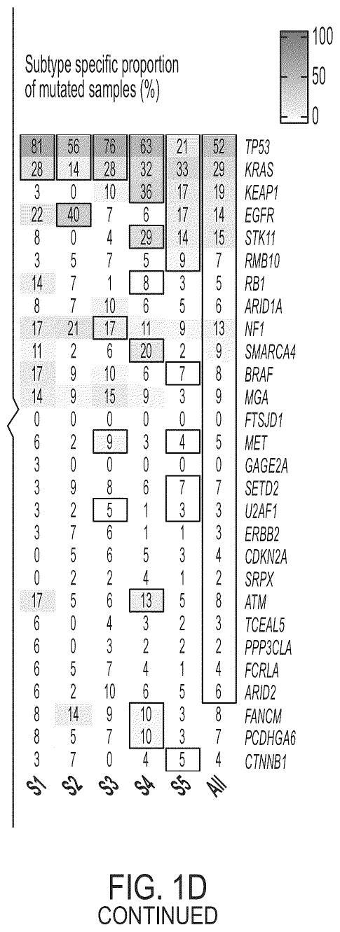

The disclosure is based, at least, in part, on findings from a powerful analysis facilitated by the integration of multiple data sets: (i) the full 509 lung adenocarcinoma (LUAD) patient cohort in TCGA; (ii) vulnerability data in lung adenocarcinoma (LUAD) cell lines from the Cancer Cell Line Encyclopedia (CCLE) lung adenocarcinoma (LUAD) cell line and Dependency Map (DepMap) repositories; and (iii) proteomic data from the Clinical Proteomic Tumor Analysis Consortium (CPTAC) cohort of lung adenocarcinoma (LUAD) patients to more precisely define therapeutically relevant lung adenocarcinoma (LUAD) subtypes (FIG. 1A). The analysis yielded distinct subtypes (S1-S5) compared with the previously published expression-based subtypes, with higher-resolution partitioning of previously defined subtypes. Moreover, experimental work in vitro linked selected subtypes with potential subtype-specific therapeutic targets, and a minimal number of biomarkers are identified that can be used in the clinic to classify patients into our most clinically relevant subtypes, which can help guide clinical decision making.

Robust lung adenocarcinoma (LUAD) subtyping can substantially aid in determining the most effective therapies that target subtype-specific vulnerabilities. Thus far, molecular therapies for lung adenocarcinoma (LUAD) have focused on targeting various genomic alterations, such as the RAS/RAF/RTK pathway. These include EGFR, ALK, and ROS1 inhibitors, as well as the recently approved targeted therapy for patients with KRAS^{G12C} mutations. Other therapies are still under development or in clinical trials (e.g., targeting MET, RET, ERBB2, NTRK1, NTRK2, and BRAF kinases). Recently, immune checkpoint blockades such as the PD-1 inhibitors, pembrolizumab and nivolumab, as well as the PD-L1 inhibitor atezolizumab, have been approved to treat lung cancer. Biomarkers of response or resistance to immunotherapy in lung adenocarcinoma (LUAD) include PD-L1 expression, tumor mutational burden (TMB), mismatch repair deficiency/microsatellite instability, and STK11 mutation. Even with these available therapies, some lung adenocarcinoma (LUAD) tumors remain untreatable, and the prognosis for many lung adenocarcinoma (LUAD) patients thus also remains poor. Therefore, more precise, and robust subtyping of lung adenocarcinoma (LUAD) tumors can help to improve prognosis and outcome for lung adenocarcinoma (LUAD) patients.

Thus, provided herein are methods of selecting a subject for treatment with a therapeutic agent (e.g., a CDK4/6 inhibitor, a c-Met inhibitor, an EGFR inhibitor, a PD-1/PD-L1 checkpoint inhibitor, and/or a TGF-beta inhibitor) based on characterization of the subject's lung adenocarcinoma subtype, S1-S5.

### Lung Cancer

Lung cancer is the most prevalent cause of death from cancer worldwide. The two major histological classes of lung cancer include: (1) non-small-cell lung cancer (NSCLC) and (2) small-cell lung cancer (SCLC). About 80-85% of lung cancers are NSCLC cancers. NSCLC is further divided into two additional subtypes including, lung adenocarcinoma (LUAD) and lung squamous cell carcinoma (LSCC, previously termed “LUSC”). LUAD evolves from mucosal glands in the lung and can be localized to the lung periphery or found in scars or areas of chronic inflammation. While smoking is a risk factor for lung adenocarcinoma, it is the most common subtype of lung cancer to be diagnosed in people who have never smoked.

Symptoms associated with lung cancer can include, but are not limited to shortness of breath, decreased breath sounds, chest pain, coughing, raspy voice, coughing up blood, wheezing, and weight loss. However, these symptoms often do not appear until the cancer has become more invasive.

Lung cancer is diagnosed by a skilled practitioner using methods known in the art, e.g., chest X-rays, computerized tomography (CT) scanning, magnetic resonance imaging (MRI), positron emission tomography (PET), sputum cytology, needle thoracentesis, lung function tests, bronchoscopy, biopsy, and blood tests.

### Lung Cancer Subtype Classification

Cancerous tumors contain mutant cells that originate from a DNA modification in a single normal cell that is then propagated through cell divisions that accumulate further DNA modifications. Patterns of somatic mutations can be uniquely ascribed to a particular mutational process or pathway in tumors that can be used to identify subtypes of a particular cancer type, e.g., lung adenocarcinoma. Classifying cancer subtypes by their mutational signature can be a useful tool for diagnosing and treating a subject with cancer or a subject that is at risk of developing cancer.

The methods and compositions provided herein relate to the identification of new and clinically useful markers for lung cancer (e.g., lung adenocarcinoma), the development of which is based upon an assessment of genomic, transcriptomic, proteomic, pathway, and survival data. The lung adenocarcinoma (LUAD) tumors classified in the working examples provided herein are identified as subtypes 1-5 or S1, S2, S3, S4, and S5.

Exemplary classifiers for the lung cancer subtypes are provided in further detail below.

### A. Genomic Classifiers

In certain aspects, the disclosure provides methods and compositions for assessment of the presence or absence of one or more sequence variants and/or mutations (e.g., structural variants including translocations (SVs), somatic copy number alterations (SCNAs) and recurrent mutations) in a test subject, tissue, cell, or sample, as compared to a corresponding reference sequence.

In particular embodiments, a subject, tissue, cell and/or sample is assessed for one or more variants and/or sites of copy number variation.

Up to five alteration types were measured and can be used for the classifier (i.e., a prognostic classifier as exemplified herein):
1.) Mutations (single nucleotide variants and/or InDels)
2.) Copy number alterations (CN gain, amplifications, CN losses, Deletions)
3.) Structural variants (chromosomal translocations, inversions, tandem duplications, etc.)
4.) Genome doublings
5.) Mutational Signatures

Mutations in Candidate Cancer Genes (CCGs), hereafter referred to as driver mutations, were identified with MutSig2CV. Representative candidate genes and/or driver mutations are provided in Tables 1-3 and 15.

Recurrent copy number alterations were identified using GISTIC2.0, as described in U.S. Patent Application Publication No. 2019/0292602, the disclosure of which is incorporated herein by reference in its entirety.

Additional methods of detecting genomic alterations in a tumor are provided, e.g., U.S. Patent Application Publication No. 2019/0078232; Bray, Freddie, et al. “Global cancer statistics 2018: GLOBOCAN estimates of incidence and mortality worldwide for 36 cancers in 185 countries.” CA: a cancer journal for clinicians 68.6 (2018): 394-424; Cancer Genome Atlas Research Network. “Comprehensive molecular profiling of lung adenocarcinoma.” Nature 511.7511 (2014): 543-550; Chen, Fengju, et al. “Multiplatform-based molecular subtypes of non-small-cell lung cancer.” Oncogene 36.10 (2017): 1384-1393; and Ghandi, Mahmoud, et al “Next-generation characterization of the cancer cell line encyclopedia.” Nature 569.7757 (2019): 503-508, the teachings of each of which are incorporated herein by reference in their entireties.

It is expressly contemplated herein that either all or a subset of these alterations discussed herein, with any combination of the individual members of each class, or even other genes, can be used within a classifier of the instant disclosure.

### B. Transcriptomic Classifiers

In certain aspects, the instant disclosure provides methods and compositions that involve and/or allow for assessment of RNA transcript abundance in a test subject, tissue, cell, or sample, as compared to a corresponding reference transcript abundance.

In some embodiments of any of the aspects, a subject, tissue, cell and/or sample is assessed for RNA transcript abundance.

Methods of measuring and analyzing RNA transcript abundance are known in the art, such as RNA sequencing (RNA-seq). For example, see, e.g., Mortazavi A, Williams B, McCue K, Schaeffer L, Wold B. Mapping and quantifying mammalian transcriptomes by RNA-Seq. Nat Methods. 2008; 5(7):621; Hoadley et al., Cell-of-Origin Patterns Dominate the Molecular Classification of 10,000 Tumors from 33 Types of Cancer. Cell. (2018) Apr. 5; 173(2):291-304; and Li, B., Dewey, C. N. RSEM: accurate transcript quantification from RNA-Seq data with or without a reference genome. BMC Bioinformatics 12, 323 (2011), the teachings of each of which are incorporated herein by reference in their entireties.

Over-expressed and/or under-expressed subtype markers in the lung cancer expression subtypes (e.g., the marker genes of Table 1) were used to determine the lung adenocarcinoma (LUAD) expression subtypes S1-S5. An RNA-Seq library can be used to determine which markers are over-expressed relative to a control sample or previously identified cancer subtypes, e.g., those described in Kim, Jaegil, et al. European Urology 75.6 (2019): 961-964, the teachings of which are incorporated herein by reference in their entirety. For example, the Cancer Cell Line Encyclopedia (CCLE) and the Clinical Proteomic Tumor Analysis Consortium (CPTAC) are libraries of RNA-seq samples that can be assigned to one of the five identified lung adenocarcinoma (LUAD) expression subtypes (S1-S5) provided herein. The association of the markers can be normalized to that of a control or reference sample. In some embodiments, the normalized association with one of the lung cancer subtypes provided herein, e.g., S1-55, is at least about 0.5, 0.55, 0.6, 0.65 or more, at least about 0.7 or more, at least about 0.8 or more, at least about 0.9 or more, at least about 0.95 or more, at least about 0.99 or more, up to 1.0.

### C. Proteomic Classifiers

In certain aspects, the disclosure provides methods and compositions that provide for assessment of polypeptide expression in a test subject, tissue, cell or sample, as compared to a corresponding reference level of polypeptide expression.

In some embodiments of any of the aspects, a subject, tissue, cell and/or sample is assessed for polypeptide expression levels or activity of a polypeptide.

Methods of characterizing polypeptide expression are known in the art, e.g., mass spectrometry, Western blotting, immunoassays, and those discussed in, Gillette, Michael A., et al. “Proteogenomic characterization reveals therapeutic vulnerabilities in lung adenocarcinoma.” Cell 182.1 (2020): 200-225; Bilal Aslam et al., Proteomics: Technologies and Their Applications. Journal of Chromatographic Science, Volume 55, Issue 2, 1 Feb. 2017, Pages 182-196; Aebersold, R. & Mann, M. Mass spectrometry-based proteomics. Nature 422, 198-207 (2003); Pandey, A., Mann, M.; Proteomics to study genes and genomes; Nature, (2000); 405(6788): 837-846; Lequin, R. M.; Enzyme Immunoassay (EIA)/Enzyme-Linked Immunosorbent Assay (ELISA); Clinical Chemistry, (2005); 51(12): 2415-2418; and Kurien, B., Scofield, R.; Western blotting; Methods (San Diego, CA), (2006); 38(4): 283-293, the teachings of each of which are incorporated herein by reference in their entireties.

### D. Pathway Classifiers

As described herein, a pathway classifier (i.e., a classification model) can be employed to characterize lung cancer subtypes (S1-S5). As would be appreciated by one of ordinary skill in the art, other forms of classification of lung cancer subtypes (e.g., nearest-neighbor, and various others) can be applied to variant and/or copy number data.

Classification models can be generated using any suitable statistical classification method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In embodiments, a supervised classification method is a recursive partitioning process. Recursive partitioning processes use recursive partitioning trees to classify data derived from unknown samples. Further details about recursive partitioning processes are provided in U.S. Patent Application No. 2002/0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from an instrument used to generate data of interest, or it may be coupled to the instrument.

The training data set and the classification models according to embodiments of the disclosure can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

In some embodiments of any of the aspects, the disclosure provided herein features pathway analysis or gene set variation analysis (GSVA). GSVA can be used to estimate variation of cell signaling pathway activity over a sample population. GSVA calculates sample-wise gene set enrichment scores as a function of genes inside and outside a given gene set, analogously to a competitive gene set test. Further, it estimates variation of gene set enrichment over the samples independently of any class label. GSVA analysis is described in further detail, e.g., in Hanzelmann, S., Castelo, R. & Guinney, J. GSVA: gene set variation analysis for microarray and RNA-Seq data. BMC Bioinformatics 14, 7 (2013), the teachings of which are incorporated by reference in its entirety.

An enrichment score for each gene set within a sample can then be assigned followed by analyzing the gene sets to identify broad biological processes. For example, gene sets can be characterized and assessed using the Molecular Signatures Database (MSigDB), available on the world wide web at gsea-msigdb.org/gsea/msigdb.

The lung cancer subtypes of the present disclosure can be identified by pathway-level activity as follows.

Subtype 2 tumors (S2) showed high pathway activity in Epithelial-mesenchymal transition (EMT) and cell adhesion pathways.

Subtype 3 tumors (S3) showed increased proliferation signatures and immune/inflammatory signatures. Specifically, S3 tumors have recurrent MET amplification and increased mRNA and protein expression of MET gene.

Subtype 4 tumors (S4) showed increased proliferation signatures, whereas subtype 5 tumors (S5) distinctively showed high levels of metabolic signatures such as lipogenesis, oxidative phosphorylation, and reactive oxygen species generation.

Lung cancer markers for subtype 3 and subtype 4 tumors are discussed further below.

### Lung Cancer Subtype Markers

The lung cancer markers provided in Table 1 can be characterized in a biological sample obtained from a subject suspected of having, at risk of developing, or that has been diagnosed with lung cancer to determine the subtype of the cancer. For example, a tissue biopsy or tumor biopsy can be obtained and characterized for the specified lung cancer subtype markers. Detecting the presence of a marker provided in Table 1 in the biological sample obtained from the subject indicates that the subject has or is at risk of having an S3 or S4 lung cancer, and should be treated with an appropriate therapy.

Lengthy table referenced here
US20240336981A1-20241010-T00001
Please refer to the end of the specification for access instructions.

### Detection of Biomarkers

The biomarkers of this disclosure can be detected by any suitable method. The methods described herein can be used individually or in combination for a more accurate detection of the biomarkers (e.g., biochip in combination with mass spectrometry, immunoassay in combination with mass spectrometry, and the like).

Detection paradigms that can be employed in the disclosure include, but are not limited to, optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described below.

Detection by Sequencing and/or Probes

In particular embodiments, the biomarkers of the disclosure are measured by a sequencing- and/or probe-based technique (e.g., RNA-seq).

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24; 109(4):1347-52). After PCR, paired-end deep sequencing can be applied to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample.

Detecting a target polynucleotide sequence or fragment thereof associated with a biomarker that hybridizes to a probe sequence may involve sequencing, FACS, qPCR, RT-PCR, a genotyping array, and/or a NanoString assay (see, e.g., Malkov, et al. “Multiplexed measurements of gene signatures in different analytes using the Nanostring nCounter™ Assay System”, BMC Research Notes, 2: Article No: 80 (2009)), or any of various other techniques known to one of skill in the art. Various detection methods may be used and are described as follows.

Preparation of a library for sequencing may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. One amplification method is PCR. In particular, the isolated RNA can be subjected to a reverse transcription assay that is coupled with a quantitative polymerase chain reaction (RT-PCR) in order to quantify the expression level of a biomarker.

Detection of the expression level of a biomarker can be conducted in real time in an amplification assay (e.g., qPCR). In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

Other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are taught, for example, in U.S. Pat. No. 5,210,015.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

In embodiments, levels of biomarkers in a sample are quantified using targeted sequencing. Methods for targeted sequencing are well known in the art (see, e.g., Rehm, “Disease-targeted sequencing: a cornerstone in the clinic”, Nature Reviews Genetics, 14:295-300 (2013)).

In embodiments, a probe comprises a molecular identifier, such as a fluorescent or chemiluminescent label, a radioactive isotope label, an enzymatic ligand, or the like. The molecular identifier can be a fluorescent label or an enzyme tag, such as digoxigenin, β-galactosidase, urease, alkaline phosphatase or peroxidase, avidin/biotin complex.

Methods used to detect or quantify binding of a probe to a target biomarker will typically depend upon the molecular identifier. For example, radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels can be detected by visualizing a colored label.

Specific non-limiting examples of molecular identifiers include radioisotopes, such as 32P, 14C, 125I, 3H, and 131I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments of the disclosure.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent bar code.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

In embodiments, the molecular identifier is a microparticle, including, as non-limiting examples, quantum dots (Empodocles, et al., Nature 399:126-130, 1999), or gold nanoparticles (Reichert et al., Anal. Chem. 72:6025-6029, 2000).

### Detection by Immunoassay

In particular embodiments, the biomarkers of the disclosure are measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This disclosure contemplates traditional immunoassays including, for example, Western blot, sandwich immunoassays including ELISA and other enzyme immunoassays, fluorescence-based immunoassays, and chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In embodiments, a sample is analyzed by means of a biochip (also known as a microarray). The polypeptides and nucleic acid molecules of the disclosure are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In embodiments, a sample is analyzed by means of a protein biochip (also known as a protein microarray). Such biochips are useful in high-throughput low-cost screens to identify alterations in the expression or post-translation modification of a biomarker, or a fragment thereof. In embodiments, a protein biochip of the disclosure binds a biomarker present in a sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the disclosure) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, CA), Zyomyx (Hayward, CA), Packard BioScience Company (Meriden, CT), Phylos (Lexington, MA), Invitrogen (Carlsbad, CA), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the disclosure, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions include, as non-limiting examples, temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In an embodiment, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In embodiments, the biomarkers of this disclosure are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the disclosure is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the disclosure to produce an array of spots on a collecting membrane, and the array may be analyzed. Subsequently, automated output of the results is provided by using an server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MSⁿ analysis to be performed. Tandem mass spectrometry allows multiple reactions to be monitored at the same time.

Capillary infusion may be employed to introduce the biomarker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high-performance liquid chromatography (HPLC) to a mass spectrometer for integrated sample separation/and mass spectrometer analysis.

Quadrupole mass analyzers may also be employed as needed to practice the disclosure. Fourier-transform ion cyclotron resonance (FTMS) can also be used for some disclosure embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the disclosure is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

A biospecific adsorbent is an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidazole, epoxide) and PG-20 (protein G coupled through acyl-imidazole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidazole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; in an embodiment, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

### Lung Cancer Marker Panels

Provided herein are panels for characterizing a biological sample by lung cancer subtype.

In some embodiments of any of the aspects provided herein, one or more polynucleotides (e.g., genes, fragments thereof, primers, probes) can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenediflumide, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

Substrates can be planar crystalline substrates such as silica based substrates (e.g. glass, quartz, or the like), or crystalline substrates used in, e.g., the semiconductor and microprocessor industries, such as silicon, gallium arsenide, indium doped GaN and the like, and include semiconductor nanocrystals.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

Silica aerogels can also be used as substrates, and can be prepared by methods known in the art. Aerogel substrates may be used as free-standing substrates or as a surface coating for another substrate material.

The substrate can take any form and typically is a plate, slide, bead, pellet, disk, particle, microparticle, nanoparticle, strand, precipitate, optionally porous gel, sheets, tube, sphere, container, capillary, pad, slice, film, chip, multiwell plate or dish, optical fiber, etc. The substrate can be any form that is rigid or semi-rigid. The substrate may contain raised or depressed regions on which an assay component is located. The surface of the substrate can be etched using known techniques to provide for desired surface features, for example trenches, v-grooves, mesa structures, or the like.

Surfaces on the substrate can be composed of the same material as the substrate or can be made from a different material, and can be coupled to the substrate by chemical or physical means. Such coupled surfaces may be composed of any of a wide variety of materials, for example, polymers, plastics, resins, polysaccharides, silica or silica-based materials, carbon, metals, inorganic glasses, membranes, or any of the above-listed substrate materials. The surface can be optically transparent and can have surface Si—OH functionalities, such as those found on silica surfaces.

The substrate and/or its optional surface can be chosen to provide appropriate characteristics for the synthetic and/or detection methods used. The substrate and/or surface can be transparent to allow the exposure of the substrate by light applied from multiple directions. The substrate and/or surface may be provided with reflective “mirror” structures to increase the recovery of light.

The substrate and/or its surface is generally resistant to, or is treated to resist, the conditions to which it is to be exposed in use, and can be optionally treated to remove any resistant material after exposure to such conditions. The substrate or a region thereof may be encoded so that the identity of the sensor located in the substrate or region being queried may be determined. Any suitable coding scheme can be used, for example optical codes, RFID tags, magnetic codes, physical codes, fluorescent codes, and combinations of codes.

In one aspect, provided herein is a panel comprising a plurality of genes selected from the group consisting of: Table 1, fragments, variants, or orthologues thereof. In some embodiments of any of the aspects, the panel provided herein comprises at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 55 or more gene or polypeptide markers provided in Table 1.

It is contemplated herein that fragments, variants, and orthologues of the human genes and/or polypeptide markers provided in Table 1 can also be used in the panel. For example, the canine ortholog of MET, Gene ID: 403438, that can be used to identify a mammal with lung cancer.

The disclosure provides methods and compositions for characterizing an S3 lung cancer subtype that involve detecting one or more markers provided in Table 2.

In some embodiments of any of the aspects, one or more of a gene and/or polypeptide marker provided in Table 2 is associated with lung adenocarcinoma subtype S3. In some embodiments of any of the aspects, the panel provided herein comprises at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40 or more gene or polypeptide markers provided in Table 2. In embodiments, the gene or polypeptide markers are selected based upon coefficients provided in any one of Tables 7-14; for example, markers having a coefficient with a magnitude or value above a particular cutoff may be selected or excluded (e.g., a cutoff of 0.01, 0.05, or 0.1), markers having a coefficient with a magnitude or value below a particular cutoff may be selected or excluded (e.g., a cutoff of 0.01, 0.05, or 0.1), and/or those markers with the highest or lowest coefficient magnitudes or values may be selected or excluded.

TABLE 2

| Gene and polypeptide makers of Subtype S3 lung adenocarcinoma | |
|---|---|
| S3 Marker | |
| No. | Marker Name |
| 1 | AFAP1L2 |
| 2 | AIM2 |
| 3 | ANNEXINVII |
| 4 | ARNTL2 |
| 5 | BATF3 |
| 6 | BRD4 |
| 7 | C10orf55 |
| 8 | C12orf70 |
| 9 | C15orf48 |
| 10 | CATSPER1 |
| 11 | CD20 |
| 12 | CD274 |
| 13 | CD70 |
| 14 | CD8A |
| 15 | CDA |
| 16 | CHK1_pS345 |
| 17 | CSF2 |
| 18 | DCBLD2 |
| 19 | DJ1 |
| 20 | ERALPHA |
| 21 | FBXO32 |
| 22 | GATA3 |
| 23 | GATA6 |
| 24 | GBP1 |
| 25 | GPR84 |
| 26 | GZMB |
| 27 | IFNG |
| 28 | JAK2 |
| 29 | KCNK12 |
| 30 | LCK |
| 31 | MET |
| 32 | MIG6 |
| 33 | MYBL1 |
| 34 | NKG7 |
| 35 | P63 |
| 36 | P70S6K1 |
| 37 | PDCD1 |
| 38 | PDL1 |
| 39 | PEA15 |
| 40 | PI3KP110ALPHA |
| 41 | PKCDELTA_pS664 |
| 42 | S100A2 |
| 43 | SYNAPTOPHYSIN |
| 44 | TBX21 |
| 45 | TGM4 |
| 46 | TIGAR |
| 47 | TMEM156 |
| 48 | TTF1 |

Additional distinguishing features of the S3 lung adenocarcinoma tumors include, e.g., amplification of the PD-L1 (CD274) gene, MET, and CDK4; FAT1 and PDE4D recurrent gene deletion and down-regulation of protein expression; increased phosphorylation levels of GAB1; increased protein expression of BCL2L1; a negative correlation between MET expression and expression of T cell effector molecules; a high level of immune signatures relative to a control sample; a higher fraction of anti-tumoral M1 macrophages as compared with S4 subtype tumors; and CDK4/6 vulnerability. High MET pathway activity was associated with the S3 lung adenocarcinoma subtype.

In another aspect, provided herein is a panel comprising one or more markers listed in Table 3 for characterizing S4 lung adenocarcinoma.

In some embodiments of any of the aspects, one or more of a gene and/or polypeptide markers provided in Table 3 is associated with lung adenocarcinoma subtype S4. In some embodiments of any of the aspects, the panel provided herein comprises at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 55, or more gene or polypeptide markers provided in Table 3. In embodiments, the gene or polypeptide markers are selected based upon coefficients provided in any one of Tables 7-14; for example, markers having a coefficient with a magnitude or value above a particular cutoff may be selected or excluded (e.g., a cutoff of 0.01, 0.05, or 0.1), markers having a coefficient with a magnitude or value below a particular cutoff may be selected or excluded (e.g., a cutoff of 0.01, 0.05, or 0.1), and/or those markers with the highest or lowest coefficient magnitudes or values may be selected or excluded.

TABLE 3

| Gene and polypeptide makers of subtype S4 lung adenocarcinoma | |
|---|---|
| S4 Marker | |
| No. | Marker Name |
| 1 | ACETYLATUBULINLYS40 |
| 2 | AKR1C2 |
| 3 | AKR1C4 |
| 4 | AMPKALPHA |
| 5 | ANNEXIN1 |
| 6 | BIM |
| 7 | C12orf39 |
| 8 | C12orf56 |
| 9 | C20orf70 |
| 10 | CALB1 |
| 11 | CALCA |
| 12 | CASPASE7CLEAVEDD198 |
| 13 | CAVEOLIN1 |
| 14 | CPS1 |
| 15 | CSAG2 |
| 16 | CYCLINB1 |
| 17 | DUSP4 |
| 18 | F2 |
| 19 | F7 |
| 20 | FOXM1 |
| 21 | GLDC |
| 22 | GNG4 |
| 23 | HEPACAM2 |
| 24 | HOXD11 |
| 25 | HOXD13 |
| 26 | IGF2BP1 |
| 27 | INSL4 |
| 28 | JNK2 |
| 29 | KCNU1 |
| 30 | KLK14 |
| 31 | LOC100190940 |
| 32 | LOC441177 |
| 33 | MIG6 |
| 34 | MLLT11 |
| 35 | MSH6 |
| 36 | MTOR_pS2448 |
| 37 | NAPSINA |
| 38 | NCADHERIN |
| 39 | NRF2 |
| 40 | P38MAPK |
| 41 | P90RSK |
| 42 | PAH |
| 43 | PCSK1 |
| 44 | PEA15 |
| 45 | PKCALPHA_pS657 |
| 46 | PKCPANBETAII_pS660 |
| 47 | POPDC3 |
| 48 | SLC38A8 |
| 49 | SYNAPTOPHYSIN |
| 50 | TFRC |
| 51 | TIGAR |
| 52 | TTF1 |
| 53 | UCHL1 |
| 54 | UGT3A1 |
| 55 | VEGFR2 |
| 56 | WDR72 |
| 57 | YAP_pS127 |
| 58 | ZMAT4 |

Additional distinguishing features of an S4 subtype lung cancer include, e.g., STK11 point mutations and indels; KRAS, SMARCA4, ATM, FANCM, and/or PCDHGA6 mutations; amplification of MET, FGFR1, and PIK3CA; FAT1 and PDE4D recurrent gene deletion; a higher fraction of pro-tumoral Th2 cells as compared with S3 tumors; c-Met inhibitor vulnerability; and CDK4/6 vulnerability.

In another aspect, provided herein is a panel comprising one or more markers listed in Table 3B for characterizing S2 lung adenocarcinoma.

In some embodiments of any of the aspects, one or more of a gene and/or polypeptide markers provided in Table 3B is associated with lung adenocarcinoma subtype S2. In some embodiments of any of the aspects, the panel provided herein comprises at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 55, or more gene or polypeptide markers provided in Table 3B. In embodiments, the gene or polypeptide markers are selected based upon coefficients provided in any one of Tables 16-19; for example, markers having a coefficient with a magnitude or value above a particular cutoff may be selected or excluded (e.g., a cutoff of 0.01, 0.05, or 0.1), markers having a coefficient with a magnitude or value below a particular cutoff may be selected or excluded (e.g., a cutoff of 0.01, 0.05, or 0.1), and/or those markers with the highest or lowest coefficient magnitudes or values may be selected or excluded.

TABLE 3B

| Gene and polypeptide makers of subtype S2 lung adenocarcinoma | |
|---|---|
| S2 Marker | |
| No. | Marker Name |
| 1 | ARAF_pS299 |
| 2 | BAP1C4 |
| 3 | BIM |
| 4 | C7orf10 |
| 5 | CAPNS2 |
| 6 | CASPASE7CLEAVEDD198 |
| 7 | CILP2 |
| 8 | CLAUDIN7 |
| 9 | CMET_pY1235 |
| 10 | COL8A2 |
| 11 | CXorf64 |
| 12 | CYCLINE1 |
| 13 | CYP26A1 |
| 14 | DBC1 |
| 15 | DIO1 |
| 16 | EGFR_pY1068 |
| 17 | ENPP3 |
| 18 | FIBRONECTIN |
| 19 | FNDC1 |
| 20 | GPR88 |
| 21 | IBSP |
| 22 | INPP4B |
| 23 | ISM1 |
| 24 | ITGA11 |
| 25 | LIPK |
| 26 | LRRTM1 |
| 27 | MAPK_pT202Y204 |
| 28 | MATN3 |
| 29 | MFAP5 |
| 30 | MMP11 |
| 31 | MYO3B |
| 32 | MYOSINIIA_pS1943 |
| 33 | P21 |
| 34 | P27 |
| 35 | P63 |
| 36 | PAXILLIN |
| 37 | PCDH19 |
| 38 | PCDH8 |
| 39 | PCNA |
| 40 | PLAT |
| 41 | PODNL1 |
| 42 | PRND |
| 43 | RANBP3L |
| 44 | SHISA3 |
| 45 | SHP2_pY542 |
| 46 | SLC24A2 |
| 47 | SMAD4 |
| 48 | SPP1 |
| 49 | ST8SIA2 |
| 50 | THBS2 |
| 51 | ZPLD1 |

Additional distinguishing features of an S4 subtype lung cancer include, e.g., EGFR mutations, and/or EGFR amplifications. In embodiments, S2 is associated with increased TGF-beta levels and/or increased M2 macrophage levels. In some embodiments, any one of the genes, polypeptides, or panels provided herein can be used to characterize, diagnose, select, and/or treat a subject with cancer or a subject that is at risk of developing cancer.

A subject is characterized as positive for one or more lung cancer marker provided herein when the marker is detected in a biological sample from the subject. In some embodiments of any of the aspects, a subject that is positive for one or more lung cancer markers selected from Table 2 identifies the subject as having or at risk of having a S3 subtype lung cancer. In some embodiments of any of the aspects, a subject that is positive for one or more lung cancer markers selected from Table 3 identifies the subject as having or at risk of having an S4 subtype lung cancer. In some embodiments of any of the aspects, a subject that is positive for one or more lung cancer markers selected from Table 3B identifies the subject as having or at risk of having an S2 subtype lung cancer. In some embodiments of any of the aspects, a subject that is positive for one or more lung cancer markers selected from Table 2, one or more markers selected from Table 3, and one or more markers from Table 3B can be tested a second time using the methods provided herein.

### Treatments

The panels and methods provided herein can be used for selecting a subject for treatment. In embodiments, a subject is administered, for example, a CDK4/6 inhibitor, a c-Met inhibitor, an EGFR inhibitor, a PD-1/PD-L1 checkpoint inhibitor, and/or a TGF-beta inhibitor. Thus, the methods provided herein include methods for the treatment of cancer, particularly lung cancer (e.g., lung adenocarcinoma). Generally, the methods provided herein include administering a therapeutically effective amount of a treatment as provided herein, to a subject who is in need of, or who has been determined to be in need of, such treatment. The treatments can be selected based upon the subtype S1-S5 of lung adenocarcinoma provided herein. Furthermore, the treatments provided herein can be highly beneficial for the treatment of drug-resistant lung tumors.

In embodiments, the panels and methods provided herein can be used for selecting a subject for inclusion in or exclusion from a clinical trial. In embodiments, the clinical trial is designed to test the efficacy of a pharmaceutical composition (e.g., a composition containing a chemotherapeutic agent, such as an inhibitor of c-Met, EGFR, PD-1/PD-L1, CDK4/6, and/or TGF-beta). The panels and methods provided herein can assist in selecting patients likely to respond to a particular agent for inclusion in a clinical trial for the study of patient response to the agent. In embodiments, the methods of the disclosure involve using the panels provided herein to separate subjects likely to respond to an agent from those likely not to respond to the agent.

The present disclosure provides a method for suggesting treatment targets. As used in this context, to “treat” means to ameliorate at least one symptom of the cancer. For example, a treatment can result in a reduction in tumor size, tumor growth, cancer cell number, cancer cell growth, or metastasis or risk of metastasis.

The methods provided herein include selecting a subject for and/or administering to a subject a treatment that includes a therapeutically effective amount of a Cdk4/6 inhibitor (e.g., abemaciclib, AT7519, CINK4, flavopiridol, palbociclib, ribociclib), and/or a c-Met inhibitor (e.g., AMG337, BMS 777607/ASLAN002, cabozantinib, capmatinib, crizotinib, emibetuzumab, ficlatuzumab, foretinib, glesatinib, onartuzumab, rilotumumab, tepotinib, tivantinib, volitinib), and/or a PD-1/PD-L1 check point inhibitor (e.g., atezolizumab, avelumab, BMS-936559, MDX-1105, cemiplimab, durvalumab, nivolumab, pembrolizumab), and/or an EGFR inhibitor (e.g., Erlotinib, Osimertinib, Neratinib, Gefitinib, Cetuximab, Panitumumab, Dacomitinib, Lapatinib, Necitumumab, Mobocertinib, and/or Vandetanib), and/or a TGF-beta inhibitor (e.g., Galunisertib, Vactosertib, Trabedersen, ISTH0036, Fresolimumab, Disitertide, Lucanix™, and/or Gemogenovatucel-T).

Therapeutic agents specifically implicated for administration for use in the treatment of the instant lung cancer subtypes provided herein include inhibitors of the following genetic targets.

c-Met

As provided herein, subtype S3 lung adenocarcinoma tumors are vulnerable to Met inhibition. The MET gene, encodes the proto-oncogene tyrosine kinase c-MET (Mesenchymal Epithelial Transition Factor), the receptor for hepatocyte growth factor (HGF). Cellular signaling via c-Met enhances cell proliferation, invasion, survival, angiogenesis, and cell motility. In tumors, increased levels of HGF and/or overexpression of c-Met are associated with poor prognosis in several solid tumors, including lung cancer (e.g., lung adenocarcinoma). Therefore, inhibitors of c-Met can be used to reduce cancer cell proliferation, survival, and metastasis.

The mechanism of c-Met oncogenesis and agents targeting c-Met and HGF for the treatment of lung cancer are further described, e.g., in Miranda, Oshin et al. “Status of Agents Targeting the HGF/c-Met Axis in Lung Cancer.” Cancers vol. 10, 9 280. 21 Aug. 2018, the teachings of which are incorporated herein by reference in its entirety.

Non-limiting examples of c-Met inhibitors that can be used in the methods provided herein include those listed in Table 4, pharmaceutical salts, analogs, derivatives, and combinations thereof.

TABLE 4
Exemplary c-Met Inhibitors

| | | | Chemical Structure or Reference |
|---|---|---|---|
| | | | to Complementarity- |
| | Company/ | | Determining Region (CDR) |
| Name | Manufacturer | Description | Sequences |
| AMG337 | AMGEN | ATP-competitive small molecule. Targets - c-Met. | 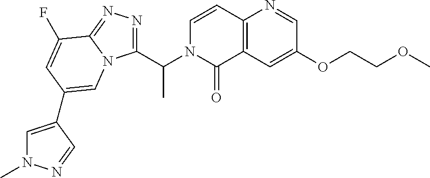 |
| BMS 777607/ ASLAN002 | BRISTOL MYERS SQUIBB COMPANY | ATP-competitive small molecule. Targets - c-Met, Axl, Tyro3, RON. | 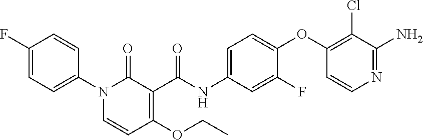 [SMILES: CCOc1ccn(-c2ccc(F)cc2)c(=O)c1C(=O)Nc1ccc(Oc2ccnc(N)c2Cl)c(F)c1] |
| Cabozantinib (XL184, CABOMETYX ® | EXELIXIS, INC. | ATP-competitive inhibitor of a wide range of kinase receptors (such as MET, vascular endothelial growth factor receptor [VEGFR], protein encoded by the rearranged during transfection oncogene [RET], tyrosine-protein kinase receptor UFO [AXL], amongst many others), blocking their autophosphorylation, which stops them from activating intracellular signaling pathways. | 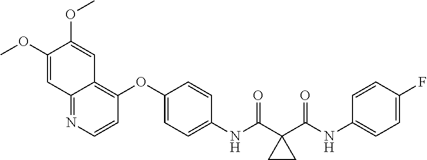 [SMILES: COc1cc2nccc(Oc3ccc(NC(=O)C4(C(=O)Nc5ccc(F)cc5)CC4)cc3)c2cc1OC] |
| | | This drug shows a high potency | |
| | | of inhibition for MET through a | |
| | | reversible effect. | |
| Capmatinib (INC280, TABRECTA ®) | NOVARTIS | ATP-competitive small molecule. Targets - c-Met. Little activity against EGFR and HER- 3. | 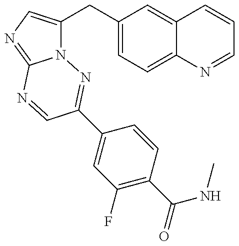 [SMILES: CNC(=O)c1ccc(-c2cnc3ncc(Cc4ccc5ncccc5c4)n3n2)cc1F] |
| Crizotinib (PF2341066, XALKORI ®) | PFIZER, INC. | Selective inhibitor of receptors, such as anaplastic lymphoma kinase (ALK), MET, and proto- oncogene tyrosine-protein kinase ROS (ROS1). This drug is considered a class I kinase inhibitor because it binds to the ATP-binding site of the receptors by forming a U-shaped loop that stabilizes the catalytically inactive conformation of each receptor. A key part of the binding of crizotinib to MET is the establishment of a π-π | 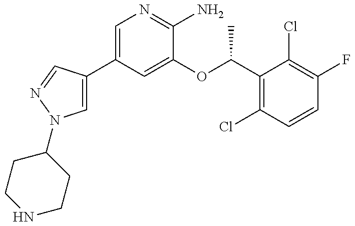 [SMILES: C[C@@H](Oc1cc(-c2cnn(C3CCNCC3)c2)cnc1N)c1c(Cl)ccc(F)c1Cl] |
| | | interaction, with the Tyr-1230 | |
| | | residue of the protein, in a dose- | |
| | | dependent manner. | |
| Emibetuzumab | ELI LILLY & | Humanized IgG4 monoclonal | See, e.g., WO 2010/059654; and |
| (LY2875358/ | COMPANY | bivalent MET antibody. It binds | See, e.g., Liu, L., et al., |
| LA480) | | to MET ECD-Fc (Fc region of | “LY2875358, a Neutralizing and |
| | | the extracellular domain) and | Internalizing Anti-MET Bivalent |
| | | does not trigger any functional | Antibody, Inhibits HGF-Dependent |
| | | agonist activities. The epitope of | and HGF-independent MET |
| | | emibetuzumab is the region of | Activation and Tumor Growth.” |
| | | the MET molecule that usually | Clinical Cancer Research, 20; |
| | | binds to hepatocyte growth | 6059 (December 2014), the |
| | | factor-beta (HGFβ). Therefore, | contents of each of which is |
| | | this drug prevents HGF from | incorporated herein by reference in |
| | | binding to MET. It also causes | their entireties. |
| | | internalization and degradation | |
| | | of the MET receptors. These | |
| | | mechanisms result in the | |
| | | blocking of ligand-dependent | |
| | | and independent HGF/MET | |
| | | signaling. | |
| Ficlatuzumab | AVEO | anti-HGF monovalent IgG1 | See, e.g., U.S. Pat. Nos. 8,580,930; |
| (AV-299/ | PHARMA- | antibody | 8,273,355; 7,943,344; and |
| SCH900105) | CEUTICALS | | 7,649,083 (Ficlatuzumab is |
| | | | identified as HE2B8-4), the |
| | | | contents of each of which is |
| | | | incorporated herein by reference in |
| | | | their entireties. |
| Foretinib (GSK1363089/ XL880) | EXELIXIS; GLAXO- SMITHKLINE plc | ATP-competitive small molecule. Targets c-Met, VEGFR-2. | 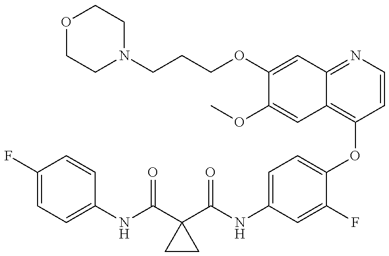 [SMILES: COc1cc2c(Oc3ccc(NC(=O)C4(C(=O)Nc5ccc(F)cc5)CC4)cc3F)ccnc2cc1OCCCN1CCOCC1] |
| Glesatinib (MGCD265) | MIRATI THERAPEUTICS INC. | ATP-competitive small molecule. Targets - c-Met, Axl. | 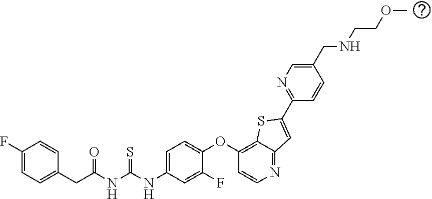 [SMILES: COCCNCc1ccc(-c2cc3nccc(Oc4ccc(NC(=S)NC(=O)Cc5ccc(F)cc5)cc4F)c3s2)nc1] |
| Onartuzumab | GENENTECH, | Recombinant humanized | See, e.g., WO2006/015371; |
| (PRO-142966, | INC. | monoclonal monovalent anti- | WO2010/04345; and Jin et al., |
| MetMAb) | | MET antibody. This molecule | Cancer Res (2008) 68:4360, the |
| | | consists of one single humanized | contents of each of which is |
| | | antigen-binding fragment (Fab) | incorporated herein by reference in |
| | | bound to a constant domain | their entireties. |
| | | fragment (Fc). Its Fab region | |
| | | binds to blades 4, 5, and 6 of the | |
| | | extracellular β-propeller “Sema” | |
| | | domain of c-MET, mainly | |
| | | through hydrogen interactions. | |
| | | The binding of onartuzumab in | |
| | | this site of the receptor blocks | |
| | | HGFα binding. The fact that | |
| | | onartuzumab is a monoclonal | |
| | | antibody prevents MET | |
| | | dimerization and, therefore, | |
| | | inhibits the activation of MET- | |
| | | related signaling pathways. | |
| Rilotumumab | AMGEN, INC. | anti-c-Met monovalent antibody | See, e.g., US 2005/0118643 and |
| (AMG-102) | | | WO 2005/017107 (Rilotumumab is |
| | | | identified as antibody 2.12.1), the |
| | | | contents of each of which is |
| | | | incorporated herein by reference in |
| | | | their entireties. |
| Tepotinib (MSC2156119J, TEPMETKO ®) | NUVISAN GMBH | ATP-competitive small molecule. Targets - c-Met. | 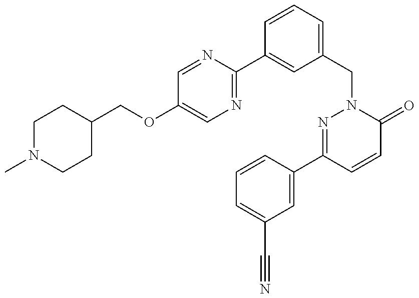 [SMILES: CN1CCC(COc2cnc(-c3cccc(Cn4nc(-c5cccc(C#N)c5)ccc4=O)c3)nc2)CC1] |
| Tivantinib (ARQ 197) | ARQULE, INC. | Small-molecule, non-adenosine triphosphate (ATP)-competitive MET inhibitor. This drug is highly selective, binding to MET only in its inactive state and causing the stabilization of the inactive molecule. The result of this is an inhibition of both intrinsic and ligand-mediated MET autophosphorylation, which halts the activation of MET-dependent signaling pathways. | 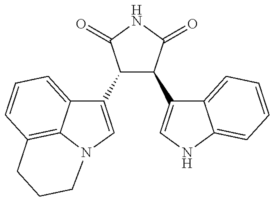 [SMILES: O=C1NC(=O)[C@@H](c2cn3c4c(cccc24)CCC3)[C@@H]1c1c[nH]c2ccccc12] |
| Volitinib/ Savolitinib (AZD6094) | ASTRAZENECA | ATP-competitive small molecule. Targets - c-Met. | 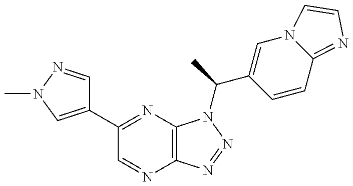 [SMILES: C[C@@H](c1ccc2nccn2c1)n1nnc2ncc(-c3cnn(C)c3)nc21] |

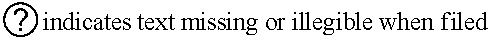

It is contemplated herein that the c-Met inhibitors of Table 4, analogs, or derivatives thereof, can be administered to a subject in combination with an additional agent, e.g., a Cdk4/6 inhibitor (e.g., abemaciclib, AT7519, CINK4, flavopiridol, palbociclib, ribociclib), and/or an additional c-Met inhibitor (e.g., AMG337, BMS 777607/ASLAN002, cabozantinib, capmatinib, crizotinib, emibetuzumab, ficlatuzumab, foretinib, glesatinib, onartuzumab, rilotumumab, tepotinib, tivantinib, volitinib), and/or a PD-1/PD-L1 check point inhibitor (e.g., atezolizumab, avelumab, BMS-936559, MDX-1105, cemiplimab, durvalumab, nivolumab, pembrolizumab).

As provided herein in the working examples, c-Met is also as a core regulator of proliferation and PD-L1 expression in the S3 lung cancer tumors. The PD-L1 pathway is discussed further below.

### PD-1/PD-L1 Pathway

The protein, Programmed Death 1 (PD-1), is an inhibitory member of the CD28 family of receptors, that also includes CD28, CTLA-4, ICOS and BTLA. PD-1 is expressed on activated B cells, T cells, and myeloid cells. The structure and function of PD-1 is further described in e.g., Okazaki et al., Curr. Opin. Immunol., 14:779-782 (2002); and Bennett et al., J. Immunol., 170:711-718 (2003), the teachings of each of which are incorporated herein by reference in their entireties.

Two ligands for PD-1 include PD-L1 (B7-H1, also called CD274 molecule) and PD-L2 (b7-DC). The PD-L1 ligand is abundant in a variety of human cancers. The interaction of PD-L1 with PD-1 generally results in a decrease in tumor infiltrating lymphocytes, a decrease in T-cell receptor mediated proliferation, and immune evasion by the cancerous cells. See, e.g., Dong et al., Nat. Med., 8:787-789 (2002); Blank et al., Cancer Immunol. Immunother., 54:307-314 (2005); and Konishi et al., Clin. Cancer Res., 10:5094-5100 (2004), the teachings of each of which have been incorporated herein by reference in their entireties.

Inhibition of the interaction of PD-1 with PD-L1 can restore immune cell activation, such as T-cell activity, to reduce tumorigenesis and metastasis, making PD-1 and PD-L1 advantageous cancer therapies. See, e.g., Yang J., e al. J Immunol. August 1: 187(3):1113-9 (2011), the teachings of which has been incorporated herein by reference in its entirety.

Non-limiting examples of PD-1/PD-L1 inhibitors that can be administered to a subject in need of treatment include those listed in Table 5.

TABLE 5
Exemplary PD-1/PD-L1 Checkpoint Inhibitors

| | | Reference to |
|---|---|---|
| | | Complementarity- |
| | | Determining Region |
| Name | Description | (CDR) Sequences |
| Atezolizumab | Atezolizumab is a humanized monoclonal | U.S. Pat. No. 8,217,149 |
| (Tecentriq, | antibody immune checkpoint inhibitor that | |
| MPDL3280A, | selectively binds to PD-L1 to stop the | |
| RG7446, | interaction between PD-1 and B7.1 (i.e., | |
| | CD80 receptors). The antibody still allows | |
| | interaction between PD-L2 and PD-1. | |
| Avelumab | Avelumab is a whole monoclonal antibody of | US 2014/0341917 |
| (Bavencio, | isotype IgG1 that binds to the programmed | |
| MSB0010718C) | death-ligand 1 (PD-L1) and therefore inhibits | |
| | binding to its receptor programmed cell death | |
| | 1 (PD-1). | |
| BMS-936559 | BMS-936559 is a human immunoglobulin | U.S. Pat. No. 7,943,743 |
| MDX-1105 | G4 (IgG4) monoclonal antibody that binds to | |
| | the PD-1 receptor and blocks its interaction | |
| | with PD-L1. | |
| Cemiplimab | Cemiplimab binds to the PD-1 receptor | U.S. Pat. No. 9,987,500 |
| (Libtayo REGN- | found on T-cells, blocking its interaction | as H4H7798N |
| 2810, REGN2810, | with PD ligand 1 (PD-L1) and PD-L2, | |
| cemiplimab-rwlc) | thereby inhibiting T-cell proliferation and | |
| | cytokine production. | |
| Durvalumab | Durvalumab is a human immunoglobulin G1 | U.S. Pat. No. 8,779,108 |
| (MEDI4736, | kappa monoclonal antibody that blocks the | |
| MEDI-4736) | interaction of PD-L1 with PD-1 and CD80 | |
| | (B7.1) to release the inhibition of immune | |
| | responses, without inducing antibody- | |
| | dependent cell-mediated cytotoxicity. | |
| Nivolumab | Nivolumab is a human immunoglobulin G4 | U.S. Pat. Nos. |
| (Opdivo | (IgG4) monoclonal antibody that binds to the | 8,952,136; 8,354,509; |
| ONO-4538, BMS- | PD-1 receptor and blocks its interaction with | 8,900,587 |
| 936558, | PD-L1 and PD-L2, releasing PD-1 pathway- | |
| MDX1106) | mediated inhibition of the immune response, | |
| | including the anti-tumor immune response. | |
| Pembrolizumab- | Pembrolizumab is a monoclonal antibody | U.S. Pat. Nos. |
| (Keytruda, MK- | that binds to the PD-1 receptor and blocks its | 8,354,509 and |
| 3475) | interaction with PD-L1 and PD-L2, releasing | 8,900,587 |
| | PD-1 pathway-mediated inhibition of the | |
| | immune response, including the anti-tumor | |
| | immune response. | |

### CDK4/6

Cyclin-dependent kinase (CDK) complexes, are protein kinases that are involved in the regulation of cell growth. These complexes comprise at least a catalytic (the CDK itself) and a regulatory (cyclin) subunit. Exemplary complexes for cell cycle regulation include cyclin A (CDK1—also known as cdc2, and CDK2), cyclin B1-B3 (CDK1) and cyclin D1-D3 (CDK2, CDK4, CDK5, CDK6), cyclin E (CDK2). Each of these complexes are involved in a particular phase of the cell cycle. In particular, CDKs that directly promote cell cycle progression include CDK4, CDK6, CDK2 and CDK1.

CDK4/6 inhibitors act on the G-to-S cell cycle checkpoint. CDK4/6 inhibitors prevent progression through this checkpoint, leading to cell cycle arrest. This checkpoint is tightly controlled by the D-type cyclins and CDK4 and CDK6. When CDK4 and CDK6 are activated by D-type cyclins, they phosphorylate the retinoblastoma-associated protein (pRb). This releases pRb's suppression of the E2F transcription factor family and allows the cell to proceed through the cell cycle and divide. Molecular mechanisms of CDKs and their inhibition in cancer therapy are discussed in detail, e.g. in O'Leary B, et al., “Treating cancer with selective CDK4/6 inhibitors.” Nat Rev Clin Oncol. 2016 July; 13(7):417-30; Asghar, Uzma et al. “The history and future of targeting cyclin-dependent kinases in cancer therapy.” Nature reviews. Drug discovery vol. 14, 2 (2015): 130-46, the teachings of each of which have been incorporated herein by reference in their entireties.

Non-limiting examples of CDK4 and CDK6 inhibitor therapies that can be administered to a subject in need of treatment include those listed in Table 6.

TABLE 6
CDK4/6 Inhibitors

| | | Chemical Structure or Reference to |
|---|---|---|
| | | Complementarity-Determining Region |
| Name | Description | (CDR) Sequences |
| Abemaciclib (Verzenio ®) | CDK inhibitor selective for CDK4 and CDK6 developed by Eli Lilly | 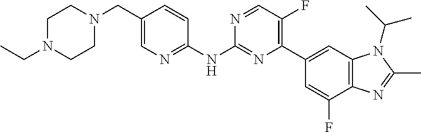 [SMILES: CCN1CCN(Cc2ccc(Nc3ncc(F)c(-c4cc(F)c5nc(C)n(C(C)C)c5c4)n3)nc2)CC1] |
| AT7519 | AT7519 is an inhibitor of Cyclin- dependent kinases (CDK). AT7519 potently inhibited CDK1,CDK2,CDK4 to CDK6, and CDK9. The compound had lower potency against other CDKs tested (CDK3 and CDK7) and was inactive against all of the non-CDK kinases tested with the exception of GSK3beta. | 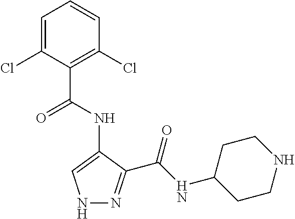 [SMILES: O=C(NC1CCNCC1)c1n[nH]cc1NC(=O)c1c(Cl)cccc1Cl] |
| Cdk4/6 Inhibitor IV (CINK4; CAS 359886-84-3) | CINK4 is a triaminopyrimidine compound that acts as a reversible and ATP-competitive inhibitor. | 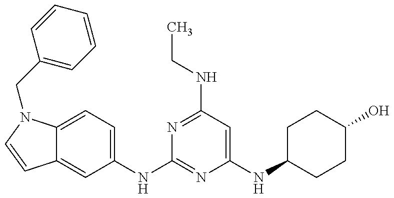 [SMILES: CCNc1cc(N[C@H]2CC[C@H](O)CC2)nc(Nc2ccc3c(ccn3Cc3ccccc3)c2)n1] |
| Flavopiridol (Alvocidib) | Flavopiridol is a pan-CDK inhibitor that acts on CDK1, 2, 4, 6, 7 and 9 at nanomolar concentrations. Has activity against Epidermal growth factor receptor tyrosine kinase and protein kinase | 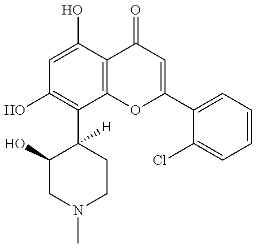 [SMILES: CN1CC[C@H](c2c(O)cc(O)c3c(=O)cc(-c4ccccc4Cl)oc23)[C@H](O)C1] |
| Palbociclib (PD-0332991 HCl/Ibrance) | It is a selective inhibitor of the cyclin- dependent kinases CDK4 and CDK6. | 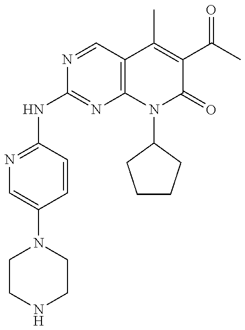 [SMILES: CC(=O)c1c(C)c2cnc(Nc3ccc(N4CCNCC4)cn3)nc2n(C2CCCC2)c1=O] |
| Ribociclib (Kisqali) | Ribociclib is an inhibitor of cyclin D1/CDK4 and CDK6. It is also being studied as a treatment for other drug- resistant cancers | 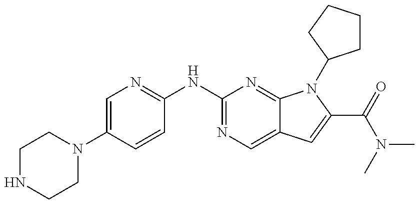 [SMILES: CN(C)C(=O)c1cc2cnc(Nc3ccc(N4CCNCC4)cn3)nc2n1C1CCCC1] |

### EGFR

As provided herein, subtype S2 lung adenocarcinoma tumors are vulnerable to EGFR inhibition. The EGFR gene, encodes the epidermal growth factor receptor, which is a transmembrane protein that is a receptor for members of the epidermal growth factor (EGF) family of extracellular protein ligands. Overexpression of EGFR is associated with various tumors. Therefore, inhibitors of EGFR can be used to reduce cancer cell proliferation, survival, and metastasis.

Non-limiting examples of EGFR inhibitors that can be used in the methods provided herein include those described below (i.e., Erlotinib, Osinertinib, Neratinib, Gefihinib, Cetuximab, Paniturnumab, Dacomitinib, Lapatinib, Necitunumab, Mobocertinib, and Vandetanib), pharmaceutical salts, analogs, derivatives, and combinations thereof.

Erlotinib (Tarceva)—Erlotinib is a tyrosine kinase receptor inhibitor that is used in the therapy of advanced or metastatic pancreatic or non-small cell lung cancer. Erlotinib is a quinazoline derivative with antineoplastic properties. Competing with adenosine triphosphate, erlotinib reversibly binds to the intracellular catalytic domain of epidermal growth factor receptor (EGFR) tyrosine kinase, thereby reversibly inhibiting EGFR phosphorylation and blocking the signal transduction events and tumorigenic effects associated with EGFR activation.

Osimertinib (Tagrisso)—Tagrisso (osimertinib) is an EGFR-TKI, a targeted cancer therapy, designed to inhibit both the activating, sensitizing mutations (EGFRm), and T790M, a genetic mutation responsible to EGFR-TKI treatment resistance. Tagrisso (osimertinib) is kinase inhibitor of the epidermal growth factor receptor (EGFR), which binds irreversibly to certain mutant forms of EGFR (T790M, L858R, and exon 19 deletion) at approximately 9-fold lower concentrations than wild-type.

Neratinib (Nerlynx)—Neratinib is a potent, irreversible tyrosine kinase inhibitor (TKI) of HER1, HER2, and HER4. Neratinib irreversibly binds to the intercellular signaling domain of HER1, HER2, HER3, and epithelial growth factor receptor, and inhibits phosphorylation and several HER downstream signaling pathways. The result is decreased proliferation and increased cell death.

Gefitinib (Iressa)—Gefitinib is an inhibitor of the epidermal growth factor receptor (EGFR) tyrosine kinase that binds to the adenosine triphosphate (ATP)-binding site of the enzyme.

Cetuximab (Erbitux)—Erbitux is a recombinant, human/mouse chimeric monoclonal antibody. The antibody binds to epidermal growth factor receptor (EGFR, HER1, c-ErbB-1) on both normal and tumor cells, and competitively inhibits the binding of epidermal growth factor (EGF) and other ligands, such as transforming growth factor-alpha. Erbitux is composed of the Fv regions of a murine anti-EGFR antibody with human IgG1 heavy and kappa light chain constant regions.

Panitumumab (Vectibix)—Vectibix binds specifically to EGFR on both normal and tumor cells, and competitively inhibits the binding of ligands for EGFR. Nonclinical studies show that binding of panitumumab to the EGFR prevents ligand-induced receptor autophosphorylation and activation of receptor-associated kinases, resulting in inhibition of cell growth, induction of apoptosis, decreased pro-inflammatory cytokine and vascular growth factor production, and internalization of the EGFR.

Dacomitinib (Vizimpro)—Vizimpro (dacomitinib) is an irreversible inhibitor of the kinase activity of the human EGFR family (EGFR/HER1, HER2, and HER4) and certain EGFR activating mutations (exon 19 deletion or the exon 21 L858R substitution mutation). In vitro dacomitinib also inhibits the activity of DDR1, EPHA6, LCK, DDR2, and MNK1 at clinically relevant concentrations.

Lapatinib (Tykerb)—Tykerb is an inhibitor of the intracellular tyrosine kinase domains of both Epidermal Growth Factor Receptor (EGFR [ErbB1]) and of Human Epidermal Receptor Type 2 (HER-2 [ErbB2]) receptors. When the binding site is blocked signal molecules can no longer attach there and activate the tyrosine kinase, an enzyme which functions to stimulate cell division.

Necitumumab (Portrazza)—Portrazza (necitumumab) is a recombinant human IgG1 monoclonal antibody that binds to the human epidermal growth factor receptor (EGFR) and blocks the binding of EGFR to its ligands. Expression and activation of EGFR has been correlated with malignant progression, induction of angiogenesis and inhibition of apoptosis. Binding of necitumumab induces EGFR internalization and degradation in vitro. In vitro, binding of necitumumab also led to antibody-dependent cellular cytotoxicity (ADCC) in EGFR-expressing cells.

Mobocertinib (Exkivity)—Exkivity (mobocertinib) is a kinase inhibitor specifically designed to selectively target epidermal growth factor receptor (EGFR) Exon20 insertion mutations at lower concentrations than wild type (WT) EGFR. Two pharmacologically-active metabolites (AP32960 and AP32914) with similar inhibitory profiles to mobocertinib have been identified in the plasma after oral administration of mobocertinib. In vitro, mobocertinib also inhibited the activity of other EGFR family members (HER2 and HER4) and one additional kinase (BLK) at clinically relevant concentrations (IC50 values <2 nM).

Vandetanib (Caprelsa)—Vandetanib is a kinase inhibitor. Vandetanib inhibits the activity of tyrosine kinases including members of the epidermal growth factor receptor (EGFR) family, vascular endothelial cell growth factor (VEGF) receptors, rearranged during transfection (RET), protein tyrosine kinase 6 (BRK), TIE2, members of the EPH receptors kinase family, and members of the Src family of tyrosine kinases. Vandetanib inhibits endothelial cell migration, proliferation, survival and new blood vessel formation in in vitro models of angiogenesis. Vandetanib inhibits EGFR-dependent cell survival in vitro. In addition, vandetanib inhibits epidermal growth factor (EGF)-stimulated receptor tyrosine kinase phosphorylation in tumor cells and endothelial cells and VEGF-stimulated tyrosine kinase phosphorylation in endothelial cells. In vivo vandetanib administration reduced tumor cell-induced angiogenesis, tumor vessel permeability, and inhibited tumor growth and metastasis in mouse models of cancer.

### TGF-Beta

As provided herein, subtype S2 lung adenocarcinoma tumors are vulnerable to TGF-beta inhibition. The TGF-beta gene, encodes transforming growth factor beta (TGF-beta), which is a multifunctional cytokine belonging to the transforming growth factor superfamily that includes three different mammalian isoforms (TGF-beta 1 to 3) and many other signaling proteins. TGF-beta polypeptides are produced by white blood cell lineages. An increase in TGF-beta often correlates with the malignancy of many tumors. Therefore, inhibitors of TGF-beta can be used to reduce the progression of cancer cell proliferation, survival, and metastasis.

Non-limiting examples of TGF-beta inhibitors that can be used in the methods provided herein include those described below (i.e., Galunisertib, Vactosertib, Trabedersen, ISTH0036, Fresolimumab, Disitertide, Lucanix™, and Gemogenovatucel-T), pharmaceutical salts, analogs, derivatives, and combinations thereof.

Galunisertib (LY2157299)—Galunisertib (LY2157299 monohydrate) is an oral small molecule inhibitor of the TGF-β receptor I kinase that specifically downregulates the phosphorylation of SMAD2, abrogating activation of the canonical pathway. Furthermore, galunisertib has antitumor activity in tumor-bearing animal models such as breast, colon, lung cancers, and hepatocellular carcinoma.

Vactosertib (TEW-7197)—Vactosertib is a potent, orally active and ATP-competitive activin receptor-like kinase 5 (ALK5) inhibitor with an IC50 of 12.9 nM. Vactosertib also inhibits ALK2 and ALK4 (IC50 of 17.3 nM) at nanomolar concentrations.

Trabedersen (AP12009)—Trabedersen is an antisense oligodeoxynucleotide complementary to a region of the mRNA of the TGF-β2 gene as an AON, exerting inhibiting effects by down-regulation of TGF-β2 mRNA.

ISTH0036—ISTH0036 is a 14-mer phosphorothioate Locked Nucleic Acid—(LNA) modified antisense oligonucleotide gapmer, targeting TGF-β2 mRNA. ISTH0036 effectively and potently downregulates target mRNA in a dose-dependent manner in relevant cell-based assays.

Fresolimumab (GC1008)—Fresolimumab is a human monoclonal antibody and an immunomodulator. GC1008 is intended for the treatment of idiopathic pulmonary fibrosis (IPF), focal segmental glomerulosclerosis, and cancer. GC1008 binds to and inhibits all isoforms of the protein transforming growth factor beta (TGF-β).

Disitertide (P144)—is a peptidic transforming growth factor-beta 1 (TGF-β1) inhibitor specifically designed to block the interaction with its receptor. Disitertide (P144) is also a PI3K inhibitor and an apoptosis inducer.

Lucanix™ (Belagenpumatucel-L)—Lucanix™ is an allogeneic cell vaccine. Lucanix™ consists of four different NSCLC lines (two adenocarcinoma, one squamous cell carcinoma, and one large cell carcinoma), thus representing a large array of antigens. The immunoadjuvant principle is based on downregulation of TGF-β2 (see above) by transfecting the cells with a TGF-β2 antisense gene.

Gemogenovatucel-T (FANG™ or vigil)—Gemogenovatucel-T is a DNA transfected autologous tumor-based immunotherapy that has three mechanisms of action: personal neoantigen education, suppression of tumor growth factor-01 and 02, and expression of granulocyte-macrophage colony-stimulating factor in the tumor microenvironment.

Additional examples of methods, agents, and combinations thereof suitable for treating lung cancer are described, e.g., Arbour K C, Riely G J. Systemic Therapy for Locally Advanced and Metastatic Non-Small Cell Lung Cancer: A Review. JAMA. 2019; 322(8):764-774; Naidoo J, et al., Immune modulation for cancer therapy. Br J. Cancer 2014; 111(12):2214-9; Asghar U, et al., The history and future of targeting cyclin-dependent kinases in cancer therapy. Nat Rev Drug Discov. 2015 February; 14(2):130-46; U.S. Pat. Nos. 6,808,710; 7,601,750 B2; 9,783,515 B2; 7,618,631 B2; 7,709,480 B2; 8,481,550 B2; 8,609,836 B2; 9,155,742 B2; 10,548,888 B2; 10,556,906 B2; 8,038,996 B2; 8,562,995 B2; 10,023,588 B2; 10,987,356 B2; 10,738,117 B2; 10,106,546 B2; 9,168,300 B2; and 9,914,771 B2, the disclosures of all of which are incorporated herein by reference in their entirety for all purposes.

In some embodiments, the agent(s) provided herein is administered in combination with an additional chemotherapeutic agent. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent (e.g., a c-Met inhibitor) may be administered only after a different therapeutic agent (e.g., a PD-1/PD-L1 inhibitor or a CDK4/6 inhibitor) has been administered and allowed to act for a prescribed period of time.

An effective amount of an agent can be administered in one or more administrations, applications or dosages. A therapeutically effective amount of a therapeutic compound or agent (i.e., an effective dosage) depends on the therapeutic compounds or agents selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic agents provided herein can include a single treatment or a series of treatments.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents which exhibit high therapeutic indices are useful. While agents that exhibit toxic side effects may be used, care should be taken to design a delivery system that targets such agents to the site of affected tissue in order to minimize potential damage to uninfected cells and, thereby, reduce side effects.

The data obtained from cell culture assays and animal studies can be used in formulating a range of dosage for use in humans. In an embodiment, the dosage of such agents lies within a range of circulating concentrations that include the ED50 with little or no toxicity. The dosage may vary within this range depending upon the dosage form employed and the route of administration utilized. For any agent used in the method of the disclosure, the therapeutically effective dose can be estimated initially from cell culture assays. A dose may be formulated in animal models to achieve a circulating plasma concentration range that includes the IC₅₀ (i.e., the concentration of the test agent which achieves a half-maximal inhibition of symptoms) as determined in cell culture. Such information can be used to more accurately determine useful doses in humans. Levels in plasma may be measured, for example, by high performance liquid chromatography.

In some embodiments, the subject is identified as having a S3 or and S4 subtype lung cancer; and the subject is administered one or more of an agent selected from the group consisting of a c-Met inhibitor (e.g., AMG337, BMS 777607/ASLAN002, cabozantinib, capmatinib, crizotinib, emibetuzumab, ficlatuzumab, foretinib, glesatinib, onartuzumab, rilotumumab, tepotinib, tivantinib, volitinib), a PD-1/PD-L1 checkpoint inhibitor (e.g., atezolizumab, avelumab, BMS-936559, MDX-1105, cemiplimab, durvalumab, nivolumab, pembrolizumab), a CDK4/CDK6 inhibitor (e.g., abemaciclib, AT7519, CINK4, flavopiridol, PD 0332991 HCl, ribociclib), and any combination thereof.

In some embodiments of any of the aspects, the subject is identified as having a S3 or S4 subtype lung cancer and the subject is administered one or more of a CDK4/6 inhibitor or a pharmaceutically acceptable salt thereof selected from the group consisting of: abemaciclib, AT7519, CINK4, flavopiridol, palbociclib, ribociclib, and any combination thereof.

In some embodiments of any of the aspects, the subject is identified as having a S3 or an S4 subtype lung cancer and the subject is administered one or more of a c-Met inhibitor or a pharmaceutically acceptable salt thereof selected from the group consisting of: AMG337, BMS 777607/ASLAN002, cabozantinib, capmatinib, crizotinib, emibetuzumab, ficlatuzumab, foretinib, glesatinib, onartuzumab, rilotumumab, tepotinib, tivantinib, volitinib, and any combination thereof.

In some embodiments of any of the aspects, the subject is identified as having a S3 subtype lung cancer and the subject is administered one or more of a PD-1 or PD-L1 checkpoint inhibitor or a pharmaceutically acceptable salt thereof selected from the group consisting of: atezolizumab, avelumab, BMS-936559, MDX-1105, cemiplimab, durvalumab, nivolumab, pembrolizumab, and any combination thereof.

### Reporting the Status

Additional embodiments of the disclosure relate to the communication of assay results or diagnoses or both to technicians, physicians or patients, for example. In certain embodiments, computers will be used to communicate assay results or diagnoses or both to interested parties, e.g., physicians and their patients. In some embodiments of any of the aspects, the assays will be performed or the assay results analyzed in a country or jurisdiction which differs from the country or jurisdiction to which the results or diagnoses are communicated.

In an embodiment, a diagnosis is communicated to the subject as soon as possible after the diagnosis is obtained. The diagnosis may be communicated to the subject by the subject's treating physician. Alternatively, the diagnosis may be sent to a subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, diagnosing of diseases, and communicating of assay results or diagnoses, may be carried out in diverse (e.g., foreign) jurisdictions.

Thus, in certain aspects, provided herein is a method of treating lung cancer, the method comprising: receiving the results of an assay that indicate that the subject has or is at risk of having an S3 or S4 lung cancer subtype provided herein; and administering to the subject an agent provided herein (e.g., the agents of Tables 4-6 and/or described above).

### Subject Management

In certain embodiments, the methods of the disclosure involve managing subject treatment based on disease status. Such management includes referral, for example, to qualified specialist (e.g., an oncologist). In one embodiment of any of the aspects, when a physician makes a diagnosis of a lung cancer (e.g., lung adenocarcinoma), then a certain regime of treatment, such as prescription or administration of therapeutic agent can follow. Alternatively, a diagnosis of non-cancer might be followed with further testing to determine a specific disease that the patient might be suffering from. Also, if the diagnostic test gives an inconclusive result on cancer status, further tests may be called for.

Additional embodiments of the disclosure relate to the communication of assay results or diagnoses or both to technicians, physicians, or patients. In certain embodiments, computers will be used to communicate assay results or diagnoses or both to interested parties, e.g., physicians and their patients. In some embodiments of any of the aspects, the assays will be performed, or the assay results analyzed in a country or jurisdiction which differs from the country or jurisdiction to which the results or diagnoses are communicated.

### Pharmaceutical Compositions

Agents of the present disclosure (e.g., a CDK4/6 inhibitor, a c-Met inhibitor, an EGFR inhibitor, a PD-1/PD-L1 checkpoint inhibitor, and/or a TGF-beta inhibitor selected from any one of Tables 4-6 and/or those agents described above), can be incorporated into a variety of formulations for therapeutic use (e.g., by administration) or in the manufacture of a medicament (e.g., for inhibiting cancer cell proliferation and survival; and/or for the treatment of lung cancer in a subject) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions provided herein can be prepared by any method known in the art of pharmacology. In general, such preparatory methods include the steps of bringing the agent or agents provided herein (e.g., a CDK4/6 inhibitor, a c-Met inhibitor, an EGFR inhibitor, a PD-1/PD-L1 checkpoint inhibitor, and/or a TGF-beta inhibitor), i.e., the “active ingredient”, into association with a carrier or excipient, and/or one or more other accessory ingredients, and then, if necessary and/or desirable, shaping, and/or packaging the product into a desired single- or multi-dose unit.

Within the scope of this disclosure is a composition that contains a suitable carrier and one or more of the therapeutic agents described above. The composition can be a pharmaceutical composition that contains a pharmaceutically acceptable carrier, a dietary composition that contains a dietarily acceptable suitable carrier, or a cosmetic composition that contains a cosmetically acceptable carrier.

The term “pharmaceutical composition” refers to the combination of an active agent with a carrier, inert or active, making the composition especially suitable for diagnostic or therapeutic use in vivo, or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound or agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, sodium lauryl sulfate, and D&C Yellow #10.

Pharmaceutically acceptable salts are salts which are, within the scope of sound medical judgment, suitable for use in contact with the tissues of humans and lower animals without undue toxicity, irritation, or allergic response, and are commensurate with a reasonable benefit/risk ratio. Pharmaceutically acceptable salts of amines, carboxylic acids, and other types of compounds and agents, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the agents of the disclosure, or separately by reacting a free base or free acid function with a suitable reagent, as described generally below. For example, a free base function can be reacted with a suitable acid. Furthermore, where the agents of the disclosure carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts, include adipate, alginate, ascorbate, aspartate, benzenesulfonate, benzoate, bisulfate, borate, butyrate, camphorate, camphor sulfonate, citrate, cyclopentane propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Representative alkali or alkaline earth metal salts include sodium, lithium, potassium, calcium, and magnesium. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl sulfonate and aryl sulfonate.

As described above, the pharmaceutical compositions of the present disclosure additionally include a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, and lubricants, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the agents of the disclosure, such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are used include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly useful; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

A pharmaceutical composition of this disclosure can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acid, such as, but not limited to, oleic acid and its glyceride derivatives, are useful in the preparation of injectables, as are natural pharmaceutically acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

In some embodiments of any of the aspects, one or more agents provided herein are formulated for oral administration. A composition for oral administration can be any orally acceptable dosage form including capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

For oral administration, the pharmaceutical compositions may take the form of, for example, tablets, lozenges, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound or agent.

Pharmaceutical compositions for topical administration according to the described disclosure can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally include one or more excipients or diluents. In some embodiments, the topical formulations include a material that would enhance absorption or penetration of the active agent(s) through the skin or other affected areas.

A topical composition contains a safe and effective amount of a dermatologically acceptable carrier suitable for application to the skin. A “cosmetically acceptable” or “dermatologically-acceptable” composition or component refers a composition or component that is suitable for use in contact with human skin without undue toxicity, incompatibility, instability, or allergic response. The carrier enables an active agent and optional component to be delivered to the skin at an appropriate concentration(s). The carrier thus can act as a diluent, dispersant, solvent, or the like to ensure that the active materials are applied to and distributed evenly over the selected target at an appropriate concentration. The carrier can be solid, semi-solid, or liquid. The carrier can be in the form of a lotion, a cream, or a gel, in particular one that has a sufficient thickness or yield point to prevent the active materials from sedimenting. The carrier can be inert or possess dermatological benefits. It also should be physically and chemically compatible with the active components provided herein, and should not unduly impair stability, efficacy, or other use benefits associated with the composition.

Pharmaceutical compositions that may oxidize and lose biological activity, especially in a liquid or semisolid form, may be prepared in a nitrogen atmosphere or sealed in a type of capsule and/or foil package that excludes oxygen (e.g., Capsugel™).

For administration by inhalation, the agents may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the agent and a suitable powder base such as lactose or starch.

Pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The agents may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The agents may also be formulated in rectal compositions such as suppositories or retention enemas, e.g., containing conventional suppository bases such as cocoa butter or other glycerides.

In addition to the formulations described previously, pharmaceutical compositions may also be formulated as a depot preparation. Such long acting formulations may be administered by implantation (for example subcutaneously or intramuscularly) or by intramuscular injection. Thus, for example, the agents may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches, e.g., transdermal patches. Patches may be used with a sonic applicator that deploys ultrasound in a unique combination of waveforms to introduce drug molecules through the skin that normally could not be effectively delivered transdermally.

Pharmaceutical compositions can contain a non-dissolving, non-disintegrating slow-release suppository base consisting essentially of a linear polymer, such as methyl cellulose, polyvinyl pyrrolidone, and water.

Pharmaceutical compositions may be incorporated into gel formulations, which generally are semisolid systems consisting of either suspension made up of small inorganic particles (two-phase systems) or large organic molecules distributed substantially uniformly throughout a carrier liquid (single-phase gels). Single-phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose, and gelatin. Although gels commonly employ aqueous carrier liquid, alcohols and oils can be used as the carrier liquid as well.

Pharmaceutical compositions may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives, and fatty alcohols. In an embodiment, emulsifier/co-emulsifier combinations are generally although not necessarily selected from the group consisting of: glyceryl monostearate and polyoxyethylene stearate; polyethylene glycol and ethylene glycol palmitostearate; and caprylic and capric triglycerides and oleoyl macrogolglycerides. The water phase includes not only water but also, typically, buffers, glucose, propylene glycol, polyethylene glycols, lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

In some embodiments of any of the aspects, a pharmaceutical formulation is provided for oral or parenteral administration, in which case the formulation may comprise an activating agent-containing microemulsion as described above, and may contain alternative pharmaceutically acceptable carriers, vehicles, additives, etc. particularly suited to oral or parenteral drug administration. Alternatively, an activating agent-containing microemulsion may be administered orally or parenterally substantially as described above, without modification.

In some embodiments of any of the aspects, the formulation comprising a compound/agent comprises one or more additional components, wherein the additional component is at least one of an osmolar component that provides an isotonic, or near isotonic solution compatible with human cells or blood, and a preservative.

In some embodiments of any of the aspects, the osmolar component is a salt, such as sodium chloride, or a sugar or a combination of two or more of these components. In some embodiments of any of the aspects, the sugar may be a monosaccharide such as dextrose, a disaccharide such as sucrose or lactose, a polysaccharide such as dextran 40, dextran 60, or starch, or a sugar alcohol such as mannitol. The osmolar component is readily selected by those skilled in the art.

In some embodiments of any of the aspects, the preservative is at least one of parabens, chlorobutanol, phenol, sorbic acid, and thimerosal.

In some embodiments of any of the aspects, the formulation comprising an agent is in the form of a sustained release formulation and optionally, further comprises one or more additional components (e.g., an anti-inflammatory agent); and a preservative.

### Methods of Administration and Dosing

Pharmaceutical compositions of the present disclosure containing an agent provided herein can be used (e.g., administered to an individual, such as a human individual, in need of treatment with a CDK4/6 inhibitor (e.g., abemaciclib, AT7519, CINK4, flavopiridol, palbociclib, ribociclib), and/or a c-Met inhibitor (e.g., AMG337, BMS 777607/ASLAN002, cabozantinib, capmatinib, crizotinib, emibetuzumab, ficlatuzumab, foretinib, glesatinib, onartuzumab, rilotumumab, tepotinib, tivantinib, volitinib), and/or an EGFR inhibitor (e.g., Erlotinib, Osimertinib, Neratinib, Gefitinib, Cetuximab, Panitumumab, Dacomitinib, Lapatinib, Necitumumab, Mobocertinib, and/or Vandetanib), and/or a PD-1/PD-L1 checkpoint inhibitor (e.g., atezolizumab, avelumab, BMS-936559, MDX-1105, cemiplimab, durvalumab, nivolumab, pembrolizumab), and/or a TGF-beta inhibitor (e.g., Galunisertib, Vactosertib, Trabedersen, ISTH0036, Fresolimumab, Disitertide, Lucanix™, and/or Gemogenovatucel-T), or an additional chemotherapeutic agent provided herein in accord with known methods, such as intravenous administration as a bolus or by continuous infusion over a period of time, oral administration, by intramuscular, subcutaneous, intratumoral, intralesional, intraperitoneal, intrapulmonary, intracerobrospinal, intracranial, intraspinal, intraarticular, intrasynovial, intrathecal, nasal, buccal, topical, rectal, or inhalation routes.

The term “parenteral” as used herein refers to subcutaneous, intracutaneous, intravenous, intramuscular, intraarticular, intraarterial, intrasynovial, intrasternal, intrathecal, intralesional, intratumoral, or intracranial injection, as well as any suitable infusion technique.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single dose) or multiple doses (e.g., multiple doses). In certain embodiments, when multiple doses are administered to a subject or applied to a tissue or cell, any two doses of the multiple doses include different or substantially the same amounts of an agent (e.g., a CDK4/6 inhibitor, a c-Met inhibitor, an EGFR inhibitor, a PD-1/PD-L1 checkpoint inhibitor, and/or a TGF-beta inhibitor selected from any one of Tables 4-6 and/or from those agents described above) provided herein.

Dosages and duration or frequency of treatment, are also envisioned to produce therapeutically useful results, i.e., a statistically significant decrease in cell proliferation and/or tumor size. It is, moreover, envisioned that localized administration to the respiratory tract (e.g., the lungs) or site of the tumor, may be optimized based on the response of cells therein (e.g., respiratory cells or cancer cells themselves).

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject, including the size, age, and general condition of the patient, the particular disorder being treated, the severity of the disorder, and the presence of other drugs in the patient. Trial dosages may be chosen after consideration of the results of animal studies and the clinical literature.

Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments of any of the aspects, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg. about 100 μg/kg, about 300 μg/kg, about 1 mg/kg. or about 2 mg/kg) may be used.

In some embodiments of any of the aspects, the agent administered to the subject in need of treatment is a c-Met inhibitor or pharmaceutically acceptable salt thereof (e.g., AMG337, BMS 777607/ASLAN002, cabozantinib, capmatinib, crizotinib, emibetuzumab, ficlatuzumab, foretinib, glesatinib, onartuzumab, rilotumumab, tepotinib, tivantinib, and/or volitinib).

In some embodiments of any of the aspects, the c-Met inhibitor (e.g., AMG337, BMS 777607/ASLAN002, cabozantinib, capmatinib, crizotinib, emibetuzumab, ficlatuzumab, foretinib, glesatinib, onartuzumab, rilotumumab, tepotinib, tivantinib, and/or volitinib) is orally or intravenously administered to the subject in an amount of at least about 100 milligrams (mg) or more, at least about 200 mg or more, at least about 300 mg or more, at least about 400 mg or more, at least about 500 mg or more, at least about 600 mg or more, at least about 700 mg or more, at least about 800 mg or more, at least about 900 mg or more, at least about 1000 mg or more, at least about 1100 mg or more, at least about 1200 mg or more, at least about 1300 mg or more, at least about 1400 mg or more, at least about 1500 mg or more, at least about 1600 mg or more, at least about 1700 mg or more, up to at least about 1800 mg.

In some embodiments of any of the aspects, the subject is orally administered AMG337 in an amount of at least about 100 milligrams (mg) or more, at least about 200 mg or more, at least about 300 mg or more, at least about 400 mg or more, at least about 500 mg or more, at least about 600 mg or more, at least about 700 mg or more, at least about 800 mg or more, at least about 900 mg or more, up to least about 1000 mg.

In some embodiments of any of the aspects, the subject is orally administered BMS 777607/ASLAN002 in an amount of at least about 1 milligram (mg) or more, at least about 10 mg or more, at least about 20 mg or more, at least about 30 mg or more, at least about 40 mg or more, at least about 50 mg or more, at least about 60 mg or more, at least about 70 mg or more, at least about 80 mg or more, at least about 90 mg or more, up to least about 100 mg.

In some embodiments of any of the aspects, the subject is orally administered cabozantinib in an amount of at least about 40 milligrams (mg) or more, at least about 60 mg or more, at least about 80 mg or more, at least about 100 mg or more, at least about 120 mg or more, at least about 160 mg or more, at least about 200 mg or more, at least about 240 mg or more, at least about 280 mg or more, at least about 320 mg or more, at least about 360 mg or more, at least about 400 mg or more, at least about 440 mg or more, at least about 480 mg or more, up to least about 520 mg.

In some embodiments of any of the aspects, the subject is orally administered capmatinib in an amount of at least about 100 milligrams (mg) or more, at least about 200 mg or more, at least about 300 mg or more, at least about 400 mg or more, at least about 500 mg or more, at least about 600 mg or more, at least about 700 mg or more, at least about 800 mg or more, at least about 900 mg or more, up to least about 1000 mg.

In some embodiments of any of the aspects, the subject is orally administered crizotinib in an amount of at least about 200 milligrams (mg) or more, at least about 400 mg or more, at least about 600 mg or more, at least about 800 mg or more, at least about 1000 mg or more, at least about 1200 mg or more, at least about 1400 mg or more, at least about 1600 mg or more, at least about 1800 mg or more, up to least about 2000 mg.

In some embodiments of any of the aspects, the subject is orally administered emibetuzumab in an amount of at least about 20 milligrams (mg) or more, at least about 40 mg or more, at least about 60 mg or more, at least about 70 mg or more, at least about 80 mg or more, at least about 210 mg or more, at least about 300 mg or more, at least about 500 mg or more, at least about 700 mg or more, at least about 1400 mg or more, at least about 2000 mg or more, up to at least about 4000 mg.

In some embodiments of any of the aspects, the subject is intravenously or orally administered ficlatuzumab in an amount of at least about 10 mg/kg or more, at least about 20 mg/kg or more, at least about 30 mg/kg or more, at least about 40 mg/kg or more, at least about 50 mg/kg or more, up to at least about 100 mg/kg.

In some embodiments of any of the aspects, the subject is orally administered foretinib in an amount of at least about 200 milligrams (mg) or more, at least about 400 mg or more, at least about 600 mg or more, at least about 800 mg or more, at least about 1000 mg or more, at least about 1200 mg or more, at least about 1400 mg or more, at least about 1600 mg or more, at least about 1800 mg or more, up to at least about 2000 mg.

In some embodiments of any of the aspects, the subject is orally administered glesatinib in an amount of at least about 200 milligrams (mg) or more, at least about 400 mg or more, at least about 600 mg or more, at least about 800 mg or more, at least about 1000 mg or more, at least about 1200 mg or more, at least about 1400 mg or more, at least about 1600 mg or more, at least about 1800 mg or more, up to at least about 2000 mg.

In some embodiments of any of the aspects, the subject is orally or intravenously administered onartuzumab in an amount of at least about 1 mg/kg or more, at least about 10 mg/kg or more, at least about 20 mg/kg or more, at least about 30 mg/kg or more, at least about 40 mg/kg or more, at least about 50 mg/kg or more, up to at least about 100 mg/kg.

In some embodiments of any of the aspects, the subject is orally or intravenously administered rilotumumab in an amount of at least about 1 mg/kg or more, at least about 1 mg/kg or more, at least about 5 mg/kg or more, at least about 7.5 mg/kg or more, at least about 15 mg/kg or more, at least about 20 mg/kg or more, up to at least about 50 mg/kg.

In some embodiments of any of the aspects, the subject is orally administered tepotinib in an amount of at least about 100 milligrams (mg) or more, at least about 200 mg or more, at least about 225 mg or more, at least about 300 mg or more, at least about 400 mg or more, at least about 450 mg or more, at least about 500 mg or more, at least about 600 mg or more, at least about 700 mg or more, at least about 800 mg or more, at least about 900 mg or more, up to least about 1000 mg.

In some embodiments of any of the aspects, the subject is orally administered tivantinib in an amount of at least about 100 milligrams (mg) or more, at least about 200 mg or more, at least about 300 mg or more, at least about 400 mg or more, at least about 500 mg or more, at least about 600 mg or more, at least about 700 mg or more, at least about 800 mg or more, at least about 900 mg or more, up to least about 1000 mg.

In some embodiments of any of the aspects, the subject is orally administered volitinib in an amount of at least about 10 milligrams (mg) or more, at least about 20 mg or more, at least about 25 mg or more, at least about 50 mg or more, at least about 100 mg or more, at least about 200 mg or more, at least about 400 mg or more, at least about 600 mg or more, at least about 800 mg or more, up to least about 1000 mg.

In some embodiments of any of the aspects, the subject is administered an EGFR inhibitor or a pharmaceutically acceptable salt thereof (e.g., Erlotinib, Osimertinib, Neratinib, Gefitinib, Cetuximab, Panitumumab, Dacomitinib, Lapatinib, Necitumumab, Mobocertinib, and/or Vandetanib).

In some embodiments of any of the aspects, the EGFR inhibitor (e.g., Erlotinib, Osimertinib, Neratinib, Gefitinib, Cetuximab, Panitumumab, Dacomitinib, Lapatinib, Necitumumab, Mobocertinib, and/or Vandetanib) is administered to the subject in an amount of at least about 100 milligrams (mg) or more, at least about 200 mg or more, at least about 300 mg or more, at least about 400 mg or more, at least about 500 mg or more, at least about 600 mg or more, at least about 700 mg or more, at least about 800 mg or more, at least about 900 mg or more, or at least about 1000 mg or more, at least about 1100 mg or more, at least about 1200 mg or more, at least about 1300 mg or more, at least about 1400 mg or more, at least about 1500 mg or more, up to at least about 1600 mg or more. In embodiments, the EGFR inhibitor is administered in an amount of at least about 0.1 mg/kg or more, at least about 0.3 mg/kg or more, at least about 0.6 mg/kg or more, at least about 0.9 mg/kg or more, at least about 1 mg/kg or more, at least about 5 mg/kg or more, at least about 10 mg/kg or more, at least about 20 mg/kg or more, and/or up to at least about 50 mg/kg.

In some embodiments of any of the aspects, the subject is administered a PD-1 or PD-L1 checkpoint inhibitor or a pharmaceutically acceptable salt thereof (e.g., atezolizumab, avelumab, BMS-936559, MDX-1105, cemiplimab, durvalumab, nivolumab, and/or pembrolizumab).

In some embodiments of any of the aspects, the PD-1 or PD-L1 checkpoint inhibitor (e.g., atezolizumab, avelumab, BMS-936559, MDX-1105, cemiplimab, durvalumab, nivolumab, and/or pembrolizumab) is orally or intravenously administered to the subject in an amount of at least about 100 milligrams (mg) or more, at least about 200 mg or more, at least about 300 mg or more, at least about 400 mg or more, at least about 500 mg or more, at least about 600 mg or more, at least about 700 mg or more, at least about 800 mg or more, at least about 900 mg or more, or at least about 1000 mg or more, at least about 1100 mg or more, at least about 1200 mg or more, at least about 1300 mg or more, at least about 1400 mg or more, at least about 1500 mg or more, up to at least about 1600 mg or more.

In some embodiments of any of the aspects, atezolizumab is orally or intravenously administered to the subject in an amount of at least about 600 mg or more, at least about 840 mg or more, at least about 1200 mg or more, at least about 1680 mg or more, up to at least about 2400 mg.

In some embodiments of any of the aspects, avelumab is orally or intravenously administered to the subject in an amount of at least about 100 mg or more, at least about 200 mg or more, at least about 400 mg or more, at least about 600 mg or more, at least about 800 mg or more, up to at least about 1000 mg.

In some embodiments of any of the aspects, the subject is orally or intravenously administered BMS-936559 in an amount of at least about 0.1 mg/kg or more, at least about 0.3 mg/kg or more, at least about 0.6 mg/kg or more, at least about 0.9 mg/kg or more, at least about 1 mg/kg or more, at least about 5 mg/kg or more, at least about 10 mg/kg or more, at least about 20 mg/kg or more, up to at least about 50 mg/kg.

In some embodiments of any of the aspects, the subject is orally or intravenously administered MDX-1105 in an amount of at least about 0.1 mg/kg or more, at least about 0.3 mg/kg or more, at least about 0.6 mg/kg or more, at least about 0.9 mg/kg or more, at least about 1 mg/kg or more, at least about 5 mg/kg or more, at least about 10 mg/kg or more, at least about 20 mg/kg or more, up to at least about 50 mg/kg.

In some embodiments of any of the aspects, the subject is intravenously administered cemiplimab in an amount of at least about 100 milligrams (mg) or more, at least about 200 mg or more, at least about 300 mg or more, at least about 350 mg or more, at least about 500 mg or more, at least about 600 mg or more, at least about 700 mg or more, at least about 800 mg or more, at least about 900 mg or more, up to least about 1000 mg.

In some embodiments of any of the aspects, durvalumab is intravenously or orally administered to the subject in an amount of at least about 0.1 mg/kg or more, at least about 0.3 mg/kg or more, at least about 0.6 mg/kg or more, at least about 0.9 mg/kg or more, at least about 1 mg/kg or more, at least about 5 mg/kg or more, at least about 10 mg/kg or more, at least about 20 mg/kg or more, up to at least about 50 mg/kg.

In some embodiments of any of the aspects, the subject is orally or intravenously administered nivolumab in an amount of at least about 100 milligrams (mg) or more, at least about 200 mg or more, at least about 300 mg or more, at least about 350 mg or more, at least about 500 mg or more, at least about 600 mg or more, at least about 700 mg or more, at least about 800 mg or more, at least about 900 mg or more, up to least about 1000 mg.

In some embodiments of any of the aspects, pembrolizumab is intravenously or orally administered to the subject in an amount of at least about 0.1 mg/kg or more, at least about 0.5 mg/kg or more, at least about 1 mg/kg or more, at least about 2 mg/kg or more, at least about 5 mg/kg or more, at least about 10 mg/kg or more, at least about 20 mg/kg or more, at least about 25 mg/kg or more, up to at least about 50 mg/kg.

In some embodiments of any of the aspects, the subject is administered a CDK4/6 inhibitor or a pharmaceutically acceptable salt (e.g., abemaciclib, AT7519, CINK4, flavopiridol, palbociclib, and/or ribociclib), wherein the CDK4/6 inhibitor is orally or intravenously administered to the subject in an amount of at least about 100 milligrams (mg) or more, at least about 200 mg or more, at least about 300 mg or more, at least about 400 mg or more, at least about 500 mg or more, at least about 600 mg or more, at least about 700 mg or more, at least about 800 mg or more, at least about 900 mg or more, at least about 1000 mg or more, at least about 1100 mg or more, at least about 1200 mg or more, at least about 1300 mg or more, at least about 1400 mg or more, at least about 1500 mg or more, at least about 1600 mg or more, at least about 1700 mg or more, at least about 1800 mg or more, at least about 1900 mg or more, up to at least about 2000 mg.

In some embodiments of any of the aspects, the abemaciclib is orally administered to the subject in an amount of at least about 100 mg or more, at least about 150 mg or more, at least about 200 mg or more, at least about 300 mg or more, at least about 400 mg or more, at least about 500 mg or more, at least about 600 mg or more, at least about 700 mg or more, up to at least about 800 mg.

In some embodiments of any of the aspects, AT7519 is intravenously administered to the subject in an amount of at least about 5 mg/kg or more, at least about 10 mg/kg or more, at least about 20 mg/kg or more, at least about 30 mg/kg or more, at least about 35 mg/kg or more, up to at least about 40 mg/kg or more,

In some embodiments of any of the aspects, CINK4, flavopiridol is intravenously administered to the subject in an amount of at least about 20 mg/kg or more, at least about 25 mg/kg or more, at least about 30 mg/kg or more, at least about 35 mg/kg or more, at least about 40 mg/kg or more, at least about 45 mg/kg or more, at least about 50 mg/kg or more, at least about 55 mg/kg or more, at least about 60 mg/kg or more, at least about 65 mg/kg or more, at least about 70 mg/kg or more, at least about 75 mg/kg or more, up to at least about 80 mg/kg.

In some embodiments of any of the aspects, the CDK4/6 inhibitor palbociclib is orally administered in an amount of at least about 25 mg or more, at least about 50 mg or more, at least about 75 mg or more, at least about 100 mg or more, at least about 125 mg or more, at least about 150 mg or more, at least about 175 mg or more, at least about 200 mg or more, at least about 250 mg or more, at least about 300 mg or more, at least about 350 mg or more, at least about 400 mg or more, at least about 450 mg or more, at least about 500 mg, at least about 1000 mg or more, at least about 2000 mg or more, up to at least about 3000 mg.

In some embodiments of any of the aspects, ribociclib is orally administered in an amount of at least about 100 mg or more, at least about 200 mg or more, at least about 300 mg or more, at least about 400 mg or more, at least about 500 mg or more, at least about 600 mg or more, at least about 700 mg or more, at least about 800 mg or more, at least about 900 mg or more, at least about 1000 mg or more, at least about 2000 mg or more, at least about 3000 mg or more, at least about 4000 mg or more, at least about 5000 mg or more, at least about 6000 mg or more, at least about 7000 mg or more, at least about 8000 mg or more, at least about 9000 mg or more, at least about 10,000 mg or more, at least about 12,000 mg or more, up to at least about 16,000 mg.

In some embodiments of any of the aspects, the subject is administered an TGF-beta inhibitor or a pharmaceutically acceptable salt thereof (e.g., Galunisertib, Vactosertib, Trabedersen, ISTH0036, Fresolimumab, Disitertide, Lucanix™, and/or Gemogenovatucel-T).

In some embodiments of any of the aspects, the TGF-beta inhibitor (e.g., Galunisertib, Vactosertib, Trabedersen, ISTH0036, Fresolimumab, Disitertide, Lucanix™, and/or Gemogenovatucel-T) is administered to the subject in an amount of at least about 100 milligrams (mg) or more, at least about 200 mg or more, at least about 300 mg or more, at least about 400 mg or more, at least about 500 mg or more, at least about 600 mg or more, at least about 700 mg or more, at least about 800 mg or more, at least about 900 mg or more, or at least about 1000 mg or more, at least about 1100 mg or more, at least about 1200 mg or more, at least about 1300 mg or more, at least about 1400 mg or more, at least about 1500 mg or more, up to at least about 1600 mg or more. In embodiments, the TGF-beta inhibitor is administered in an amount of at least about 0.1 mg/kg or more, at least about 0.3 mg/kg or more, at least about 0.6 mg/kg or more, at least about 0.9 mg/kg or more, at least about 1 mg/kg or more, at least about 5 mg/kg or more, at least about 10 mg/kg or more, at least about 20 mg/kg or more, and/or up to at least about 50 mg/kg.

The agent provided herein (e.g., a CDK4/6 inhibitor, a c-Met inhibitor, an EGFR inhibitor, a PD-1/PD-L1 checkpoint inhibitor, and/or a TGF-beta inhibitor) can be administered to the subject in an amount sufficient to achieve a desired effect at a desired site (e.g., inhibition of cancer cell proliferation or growth, reduction of cancer size, reduction in cancer cell abundance, symptoms, etc.) as determined by a skilled clinician to be effective.

In some embodiments of the disclosure, the agent is administered at least about once a year. In other embodiments of the disclosure, the agent is administered at least about once a day, at least about twice a day, at least about three times a day, at least about four times a day, up to at least about five times a day. In other embodiments of the disclosure, the agent is administered at least about once a week or at least about twice a week or more. In some embodiments of the disclosure, the agent is administered at least about once a month.

In certain embodiments, dosing frequency is at least about three times per day, is at least about twice per day, is at least about once per day, is at least about once every other day, is at least about once weekly, is at least about once every two weeks, is at least about once every four weeks, is at least about once every five weeks, is at least about once every six weeks, is at least about once every seven weeks, is at least about once every eight weeks, is at least about once every nine weeks, is at least about once every ten weeks, or is at least about once monthly, is at least about once every two months, is at least about once every three months, or longer. Progress of the therapy is easily monitored by conventional techniques and assays. The dosing regimen, including the agent(s) administered, can vary over time independently of the dose used.

Therapeutic efficacy of a compound/agent and/or compositions comprising the same may be determined by evaluating and comparing patient symptoms and quality of life pre- and post-administration. Such methods apply irrespective of the mode of administration. In a particular embodiment, pre-administration refers to evaluating patient symptoms and quality of life prior to onset of therapy and post-administration refers to evaluating patient symptoms and quality of life at least 1, 2, 3, 4, 5, 6, 7, 8, 9, 10, 11, 12, 14, 14, 15, 16, 17, 18, 29, 20 weeks after onset of therapy. In a particular embodiment, the post-administration evaluating is performed about 2-8, 2-6, 4-6, or 4 weeks after onset of therapy. In a particular embodiment, patient symptoms (e.g., gastrointestinal upset) and quality of life pre- and post-administration are evaluated clinically and by questionnaire assessment.

### Efficacy of Treatment

The therapeutic agents and combinations thereof featuring a CDK4/6 inhibitor, a c-Met inhibitor, an EGFR inhibitor, a PD-1/PD-L1 inhibitor, a TGF-beta inhibitor, and/or another chemotherapeutic are useful for treating lung cancer (e.g., an S3 or S4 lung cancer subtype). The compositions and methods provided herein can be used to reduce cancer cell proliferation or survival in vivo or in vitro.

Methods of evaluating tumor progression or cell proliferation are well known in the art. The methods provided herein result in a reduction in the proliferation or survival of cancer cells. For example, after treatment with one or more of the agents provided herein, cancer cell proliferation or survival is reduced by 5% or greater (e.g., 10%, 20%, 30%, 40%, 50%, 60%, 70%, 80%, 90% or greater) relative to cell proliferation or survival prior to treatment.

The methods provided herein can result in a reduction in size or volume of a tumor. For example, after treatment, tumor size is reduced by 5% or greater (e.g., 10%, 20%, 30%, 40%, 50%, 60%, 70%, 80%, 90% or greater) relative to its size prior to treatment. Size of a tumor may be measured by any reproducible means of measurement. The size of a tumor may be measured as a diameter of the tumor or by any reproducible means of measurement.

Treating cancer can further result in a decrease in number of tumors. For example, after treatment, tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement. The number of tumors may be measured by counting tumors visible to the naked eye or at a specified magnification (e.g., 2×, 3×, 4×, 5×, 10×, or 50×).

Selecting a Subject for Treatment with a CDK4/6 Inhibitor, a c-Met Inhibitor, an EGFR Inhibitor, a PD-1/PD-L1 Inhibitor, and/or a TGF-Beta Inhibitor

The disclosure features methods of selecting cancer therapy for a subject that has or is at risk of developing lung cancer (e.g., lung adenocarcinoma) that is characterized as having a particular subtype, S1-S5.

The methods provided herein comprise characterizing, measuring, or detecting the presence or absence, expression levels, activity, and/or sequence of one or more markers selected from Table 1, Table 2, Table 3, or Table 3B in a biological sample (e.g., a tumor, blood sample, cfDNA sample) and selecting the subject for treatment when the presence, levels, activity, or sequence of one or more markers selected from Table 1, Table 2, Table 3, or Table 3B are altered relative to a reference level.

Accordingly, this disclosure provides for the characterization of a biological sample from a subject having or suspected of having lung cancer (e.g., lung adenocarcinoma). Such characterization includes characterizing a polynucleotide or polypeptide marker from Table 1, Table 2, Table 3, or Table 3B in a biological sample obtained from a subject and detecting the presence or absence of a polynucleotide or polypeptide marker from Table 1, Table 2, Table 3, or Table 3B wherein detection of the presence of a polynucleotide or polynucleotide marker from Table 1, Table 2, Table 3, or Table 3B selects the subject for treatment with a therapeutic agent provided herein, e.g., a CDK4/6 inhibitor, a c-Met inhibitor, an EGFR inhibitor, a PD-1/PD-L1 checkpoint inhibitor, a TGF-beta inhibitor, and/or an additional chemotherapeutic agent.

Thus, in some embodiments, when the levels of the one or more markers selected from Table 1, Table 2, Table 3, or Table 3B are present relative to a control sample or reference level, the subject will be selected for treatment. In some embodiments of any of the aspects, when the levels of the one or more markers selected from Table 1, Table 2, Table 3, or Table 3B are altered relative to a control sample or reference level, the subject will be selected for treatment. In some embodiments of any of the aspects, when the activity of the one or more markers selected from Table 1, Table 2, Table 3, or Table 3B are altered relative to a control sample or reference level, the subject will be selected for treatment. In some embodiments of any of the aspects, when the polynucleotide sequence of the one or more markers selected from Table 1, Table 2, Table 3, or Table 3B are altered relative to a reference sequence, the subject will be selected for treatment.

In some embodiments of any of the aspects, the markers of Table 2 are altered, indicating that the subject has an S3 lung cancer subtype. In some embodiments of any of the aspects, the markers of Table 3 are altered, indicating that the subject has an S4 lung cancer subtype. In some embodiments of any of the aspects, the markers of Table 3B are altered, indicating that the subject has an S2 lung cancer subtype.

Methods of characterizing, evaluating, and quantifying the level or activity of a marker are discussed further above and in the working examples.

### Classification Algorithms

The present disclosure provides methods for characterizing a lung cancer, e.g., lung adenocarcinoma, as belonging to a particular subtype (e.g., S1-S5). The expression subtype is useful in predicting clinical outcome and/or for guiding therapy.

In some embodiments, data derived from the assays for detection of biomarkers (e.g., RNA-seq) that are generated using samples such as “known samples” can then be used to “train” a classification model. Exemplary methods for developing a model for classifying a lung adenocarcinoma as belonging to a subtype are described in the Tables and the Examples provided herein. A “known sample” is a sample that has been pre-classified. The data used to form the classification model can be referred to as a “training data set.” Once trained, the classification model (e.g., a machine learning classifier) can be used to classify the expression subtype of a lung adenocarcinoma based upon levels of biomarkers detected in a sample. The sample can be taken from a subject having lung adenocarcinoma. This can be useful, for example, in guiding selection of a treatment for a subject or for prognostic purposes.

The training data set that is used to form the classification model may comprise raw data or pre-processed data. In embodiments, a classifier can be trained using a random forest classifier, as described in the Examples provided herein.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In embodiments, a supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from a device that is used to detect biomarkers, or it may be coupled to the device.

The training data set and the classification models according to embodiments of the disclosure can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

### Hardware and Software

The present disclosure also provides a computer system useful in analyzing data associated with a marker (e.g., biomarker expression), patient selection, and related computations (e.g., calculations associated with a machine learning classifier).

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

In aspects, software used to analyze the data can include code that applies an algorithm to the analysis of the results. The software also can also use input data (e.g., sequence data or biochip data) to classify a lung cancer (e.g., lung adenocarcinoma).

### Kits

The instant disclosure also provides kits containing agents of this disclosure for use in the methods of the present disclosure. Kits of the instant disclosure may include one or more containers comprising an agent for treatment of lung cancer and/or may contain agents (e.g., oligonucleotide primers, probes, etc.) for identifying a cancer or subject as possessing one or more variant sequences. In some embodiments of any of the aspects, the kits further include instructions for use in accordance with the methods of this disclosure. In some embodiments of any of the aspects, these instructions comprise a description of use of the agent to treat or diagnose, e.g., lung cancer, according to any of the methods of this disclosure. In some embodiments of any of the aspects, the instructions comprise a description of how to detect a lung cancer subtype, for example in an individual, in a tissue sample, or in a cell. The kit may further comprise a description of treatments suggested for an individual as suitable for treatment based on identifying whether that subject has a specific subtype of lung cancer (e.g., lung adenocarcinoma).

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods provided herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present disclosure employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

### Example 1: Genomic Characterization Reveals Five Lung Adenocarcinoma (LUAD) Expression Subtypes

Two of the largest published studies on lung adenocarcinoma (LUAD) subtypes are: (i) the original The Cancer Genome Atlas (TCGA) LUAD subtyping paper published in 2014 that used 230 patients (the largest number of patients available at the time) to identify three subtypes based on mRNA expression—Proximal Inflammatory (PI), Proximal Proliferative (PP), and Terminal Respiratory Unit (TRU) (Cancer Genome Atlas Research Network. “Comprehensive molecular profiling of lung adenocarcinoma.” Nature 511.7511 (2014): 543-550); and (ii) a ‘cluster-of-clusters analysis’ (COCA)-based study in 2017 that applied a multi-omics approach on a combined LSCC and LUAD cohort to identify 6 subtypes (Chen, Fengju, et al. “Multiplatform-based molecular subtypes of non-small-cell lung cancer.” Oncogene 36.10 (2017): 1384-1393). The TCGA LUAD cohort has increased to a total of 509 LUAD cases, offering the possibility to perform sufficiently powered analyses that can increase the resolution of the subtypes and refine the LUAD subtypes beyond TCGA's original study.

Expression data are the most predictive genomic features of cancer dependencies. Therefore, a consensus clustering approach (see, e.g., Taylor-Weiner, Amaro, et al. “Scaling computational genomics to millions of individuals with GPUs.” Genome biology 20.1 (2019): 1-5) was applied, using Bayesian Non-Negative Matrix Factorization (BayesianNMF) on the expression data representing the 509 LUAD cases from TCGA. The analysis revealed a robust and detailed structure, yielding five lung adenocarcinoma (LUAD) expression subtypes, designated as S1 to S5 (FIG. 1A).

To further explore the expression subtypes, they were compared to the previously-defined LUAD expression subtypes—PI, PP, and TRU (Cancer Genome Atlas Research Network. “Comprehensive molecular profiling of lung adenocarcinoma.” Nature 511.7511 (2014): 543-550). Among the 230 TCGA lung adenocarcinoma (LUAD) tumors with information available for these three subtypes, it was found that S5 was most closely related to the TRU subtype (77.4% of S5 tumors were from the TRU subtype, and 80.9% of TRU subtype mapped to S5 [Fisher's exact test P=3.3×10⁻²⁴]), and S4 was enriched with the PP subtype (76.4% of S4 were PP; P=9.4×10⁻⁹). The S1, S2, and S3 subtypes mostly matched the PI subtype. Of these, S3 was the most enriched with the PI subtype (85.7% of S3 tumors matched to PI; Fisher's exact test P=1.7×10⁻¹⁴) (FIG. 1B).

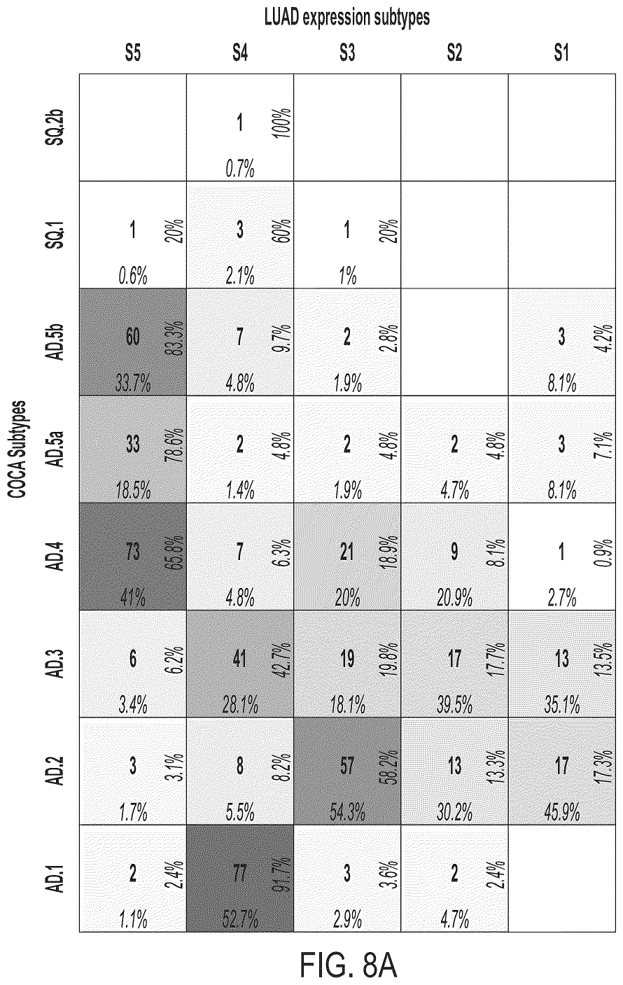

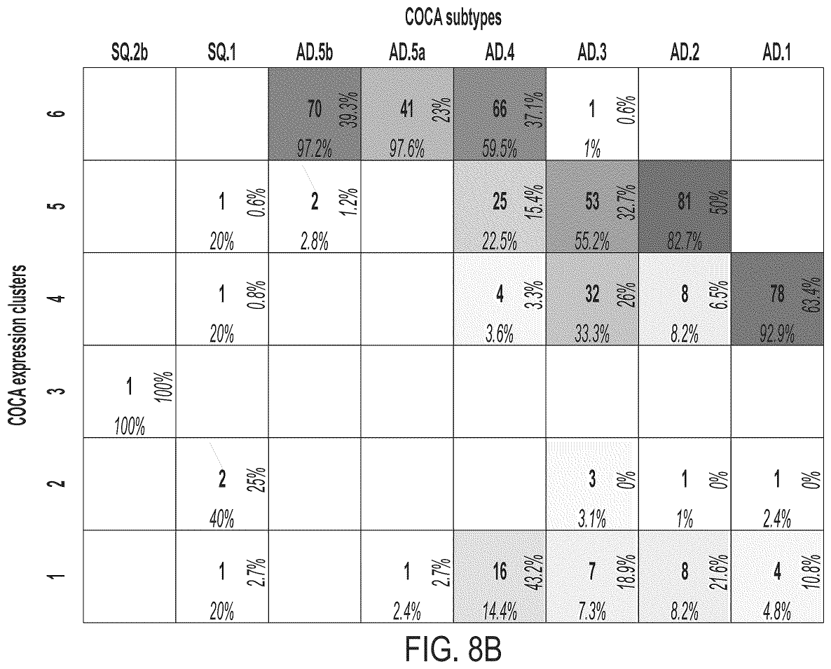

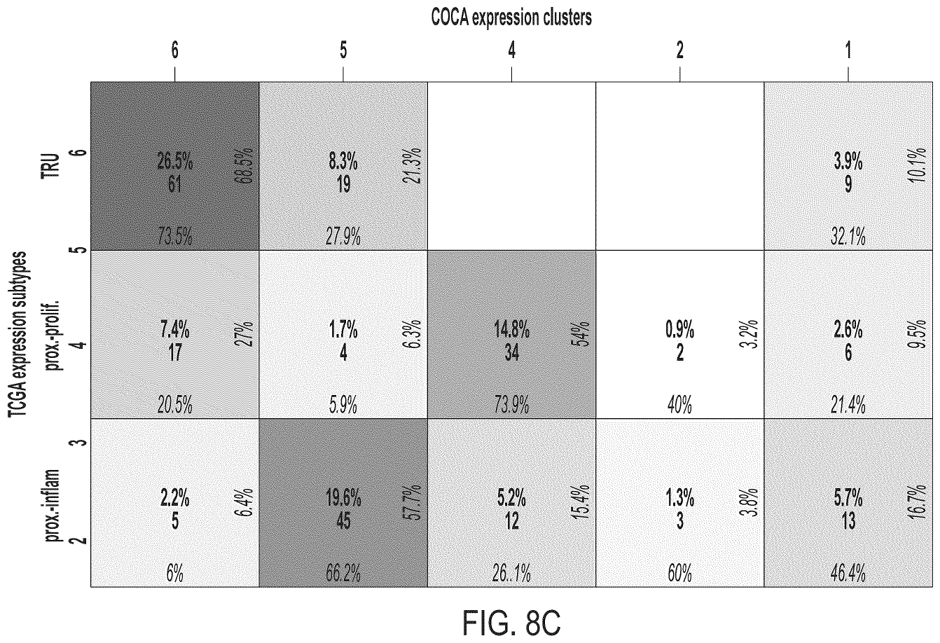

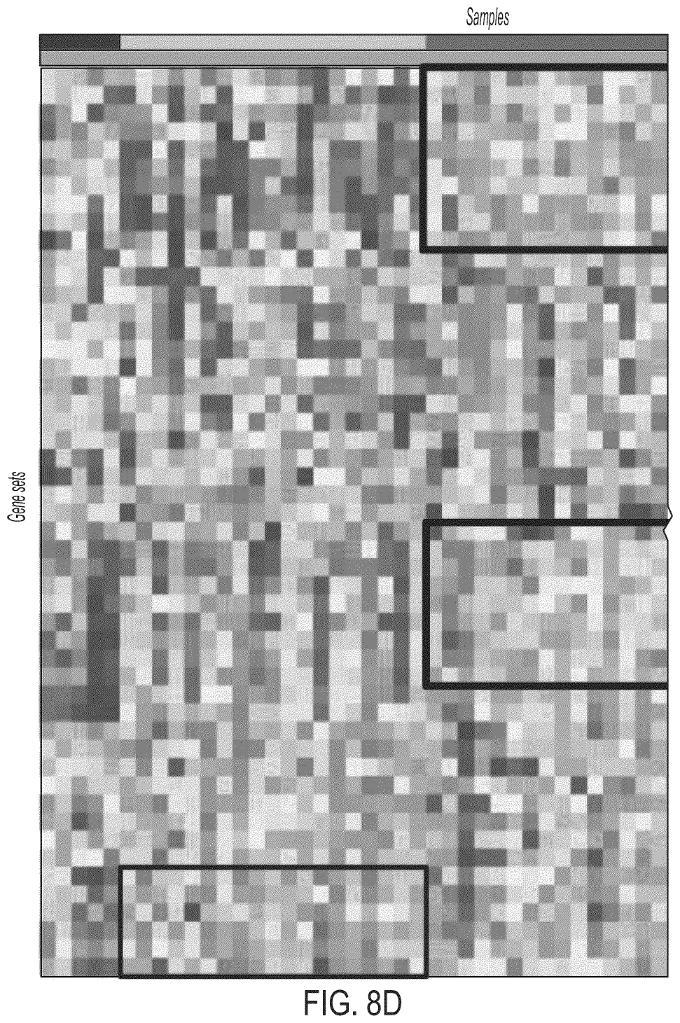

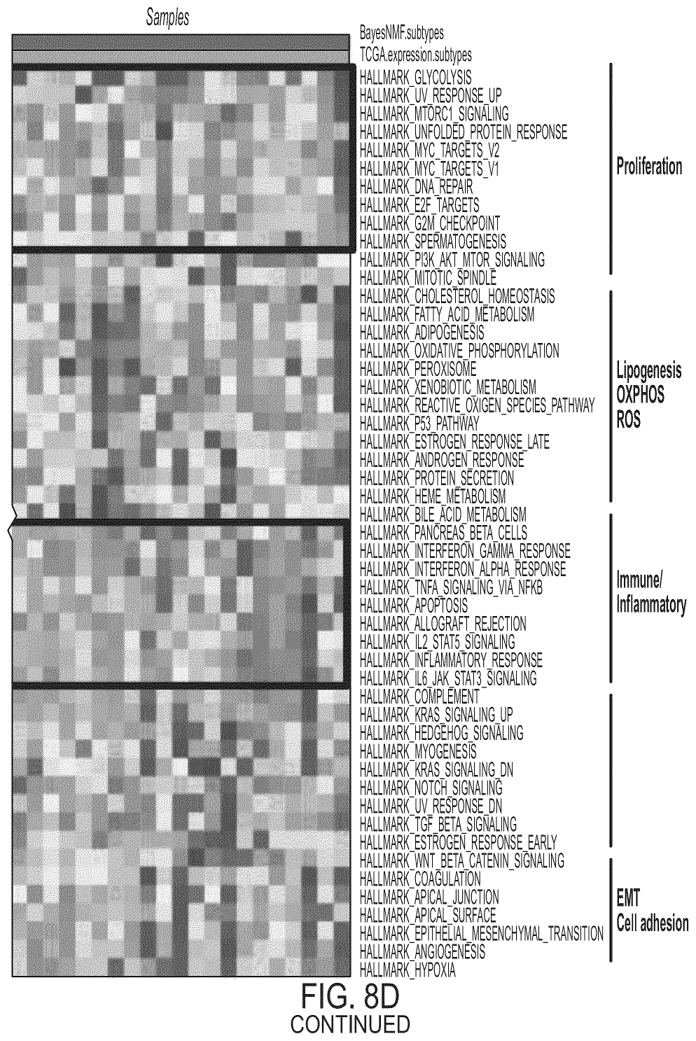

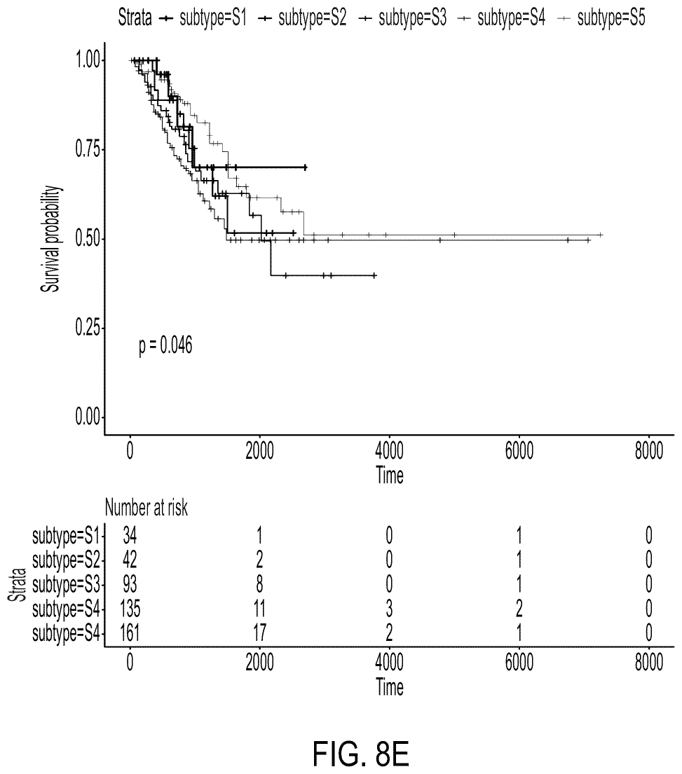

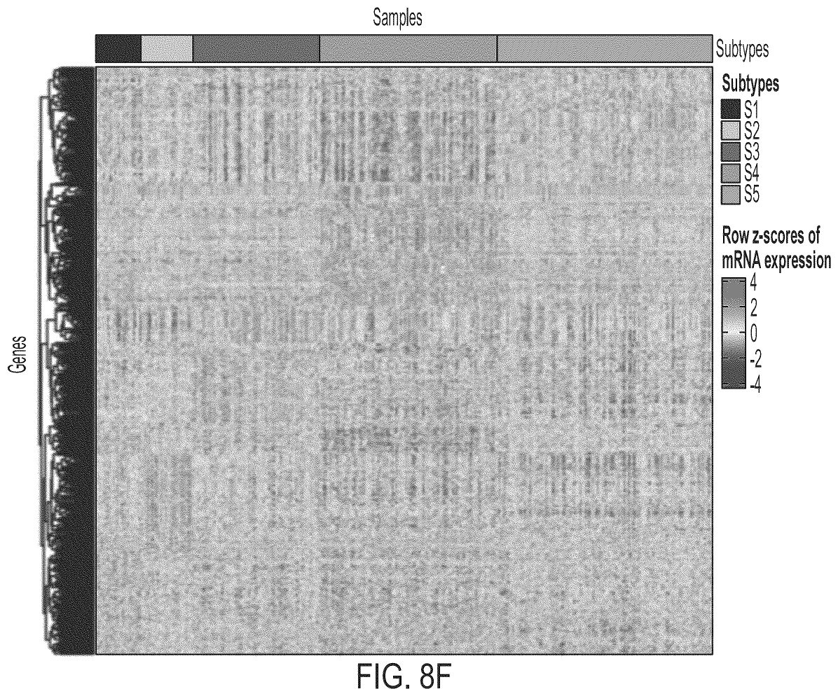

Following this analysis, the expression subtypes were also compared to Cluster of Clusters Analysis (COCA)-based subtypes (Chen, Fengju, et al. “Multiplatform-based molecular subtypes of non-small-cell lung cancer.” Oncogene 36.10 (2017): 1384-1393; “Chen”) and low concordance was initially found (FIG. 8A). This low concordance was not surprising since the Cluster of Clusters Analysis (COCA) subtypes were defined across all non-small-cell lung cancer (NSCLC) (including both LSCC and lung adenocarcinoma (LUAD)) tumors and were obtained by first clustering these tumors based on different genomic features (DNA copy number, DNA methylation, mRNA expression, miRNA expression, and protein) and then further clustering tumors based on Chen's cluster assignments across the different features. An analysis was therefore performed on only Chen's intermediate clustering of mRNA expression data containing 6 clusters, which were significantly different from Chen's final Cluster of Clusters Analysis (COCA) subtypes (FIG. 8B). Indeed, when these 6 mRNA-based clusters were compared with the 5 mRNA-based subtypes as well as the previously published PP, PI, and TRU expression-based subtypes, observed a high concordance was observed among them all (FIG. 1B). The majority of lung adenocarcinoma (LUAD) samples (91%) were assigned to Chen's clusters 4, 5, and 6, and these three clusters also mapped to the subtypes PP, PI, and TRU, respectively (FIG. 8C). Comparing directly to the five subtypes provided herein, 74.7% of the S4 tumors mapped to Chen's cluster 4, while 83.7% of the S5 tumors mapped to Chen's cluster 6. Moreover, Chen's cluster 5 could be further partitioned to the S1, S2, and S3 clusters provided herein, which is consistent with cluster 5 of Chen mapping to PI and aligning with the results showing that PI was also partitioned into S1, S2, and S3 (FIG. 1B).

To explore the biological differences among the five subtypes (S1-S5), pathway activity levels were calculated for each lung adenocarcinoma (LUAD) sample using single-sample gene set variance analysis (GSVA) on the Molecular Signatures Database (MSigDB) hallmark gene sets in order to identify the pathways with significantly different activities across the subtypes. S1 showed a low immune/inflammatory signature, and S2 showed high pathway activity in epithelial-mesenchymal transition (EMT) and cell-adhesion pathways. Both S3 and S4 showed increased proliferation signatures, but only S3 showed high immune/inflammatory signatures. S5 distinctively showed low proliferation signatures (FIG. 1C).

To further support the partitioning of the 60 tumors that originally were assigned to the PI subtype and were further partitioned in the S1, S2 and S3 subgroups, a search was undertaken for differences in pathway activities among them. A consistent set of differentially active pathways was found as determined when using all the samples (e.g., low immune/inflammatory signature in S1; high EMT in S2; high E2F, MYC targets, G2M markers, and interferon alpha/gamma response in S3) (FIGS. 8D and 1C). These findings were consistent with the results provided herein revealing novel, biologically distinct subtypes that were previously grouped together within the single PI subtype.

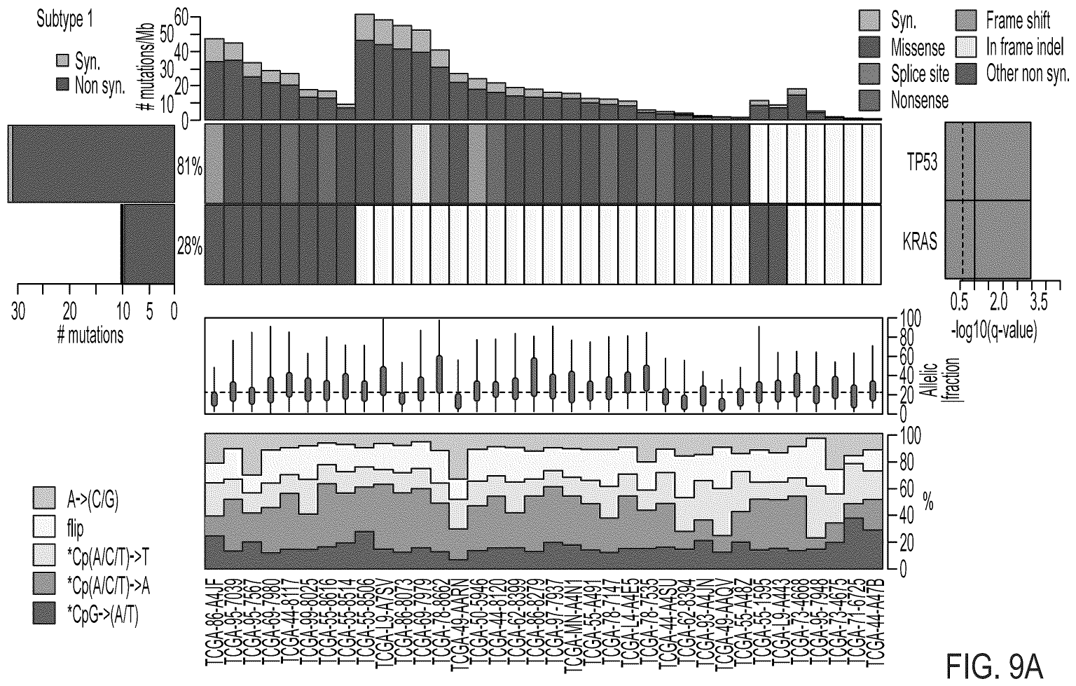

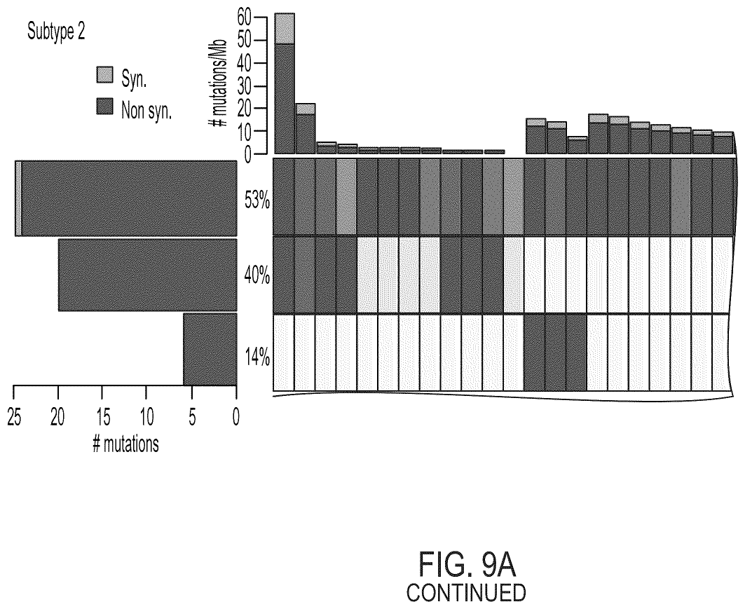

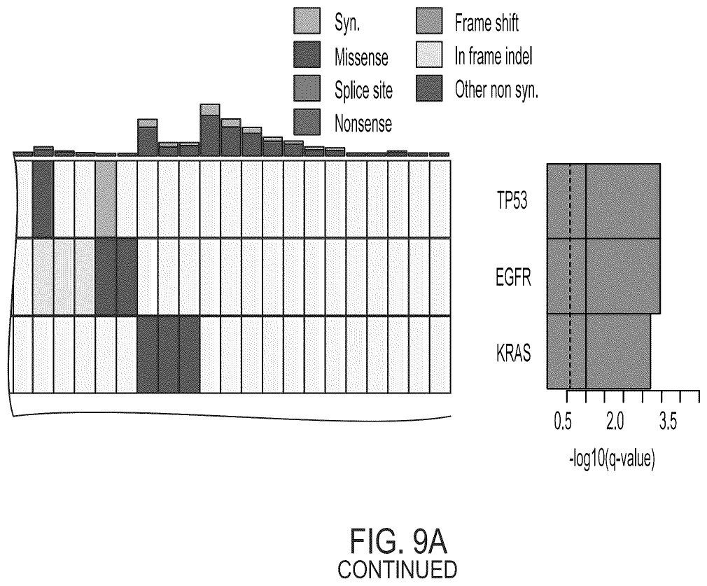

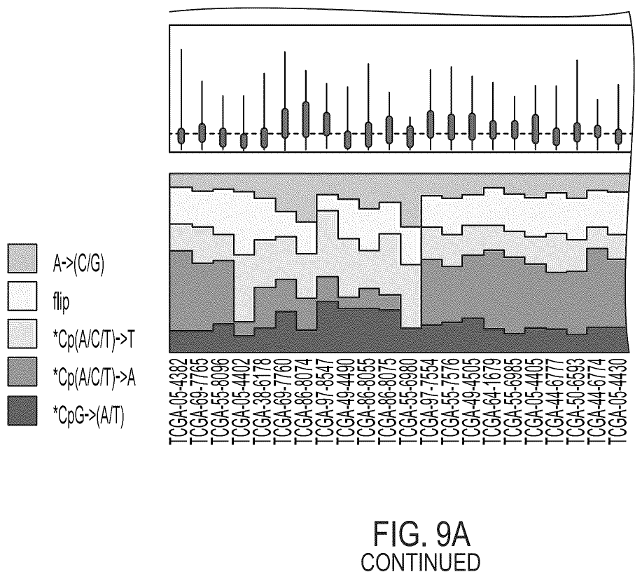

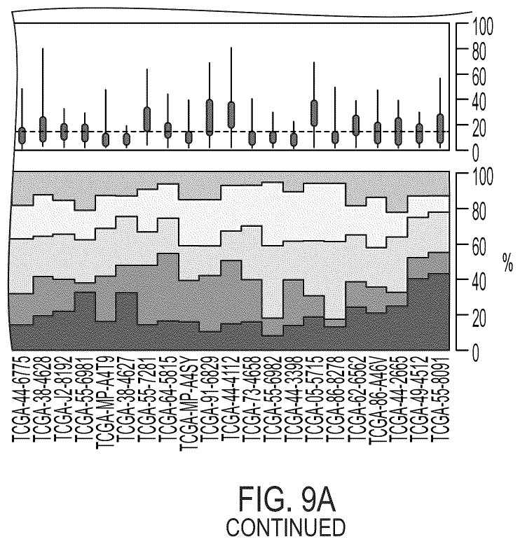

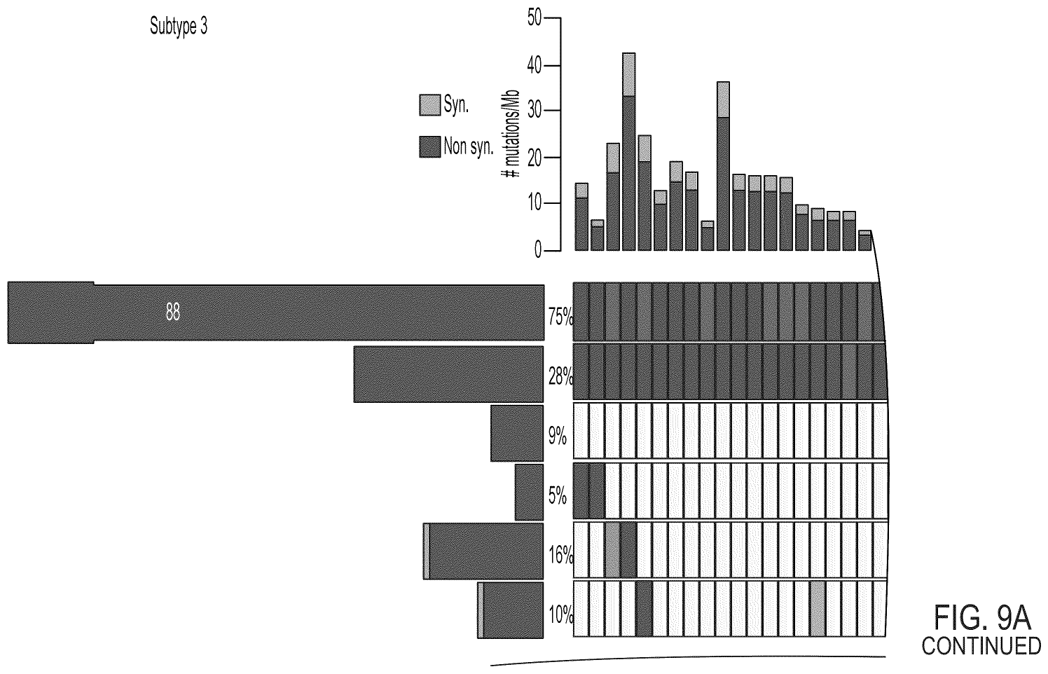

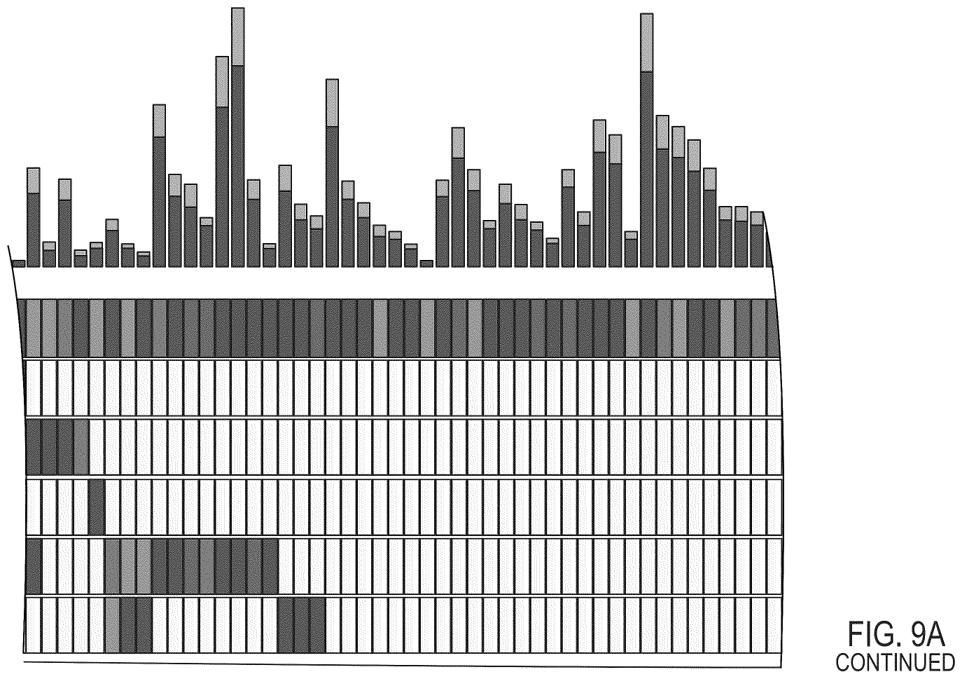

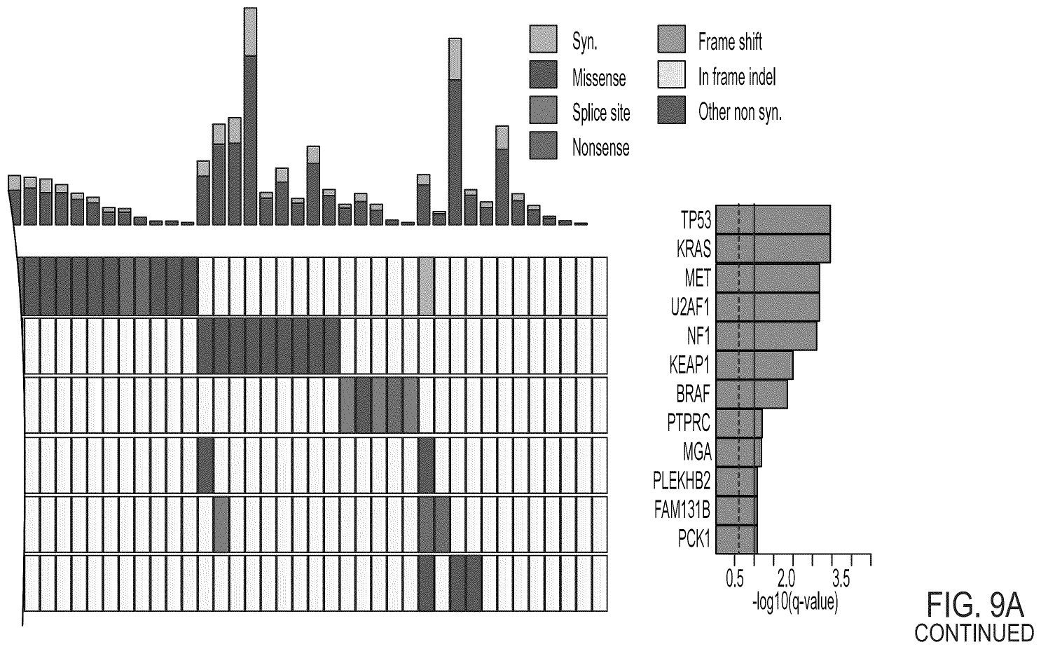

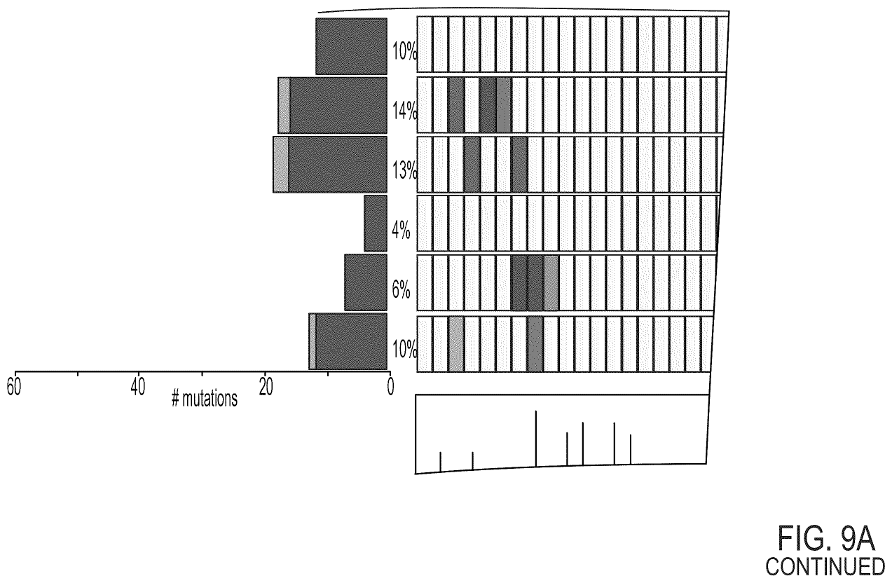

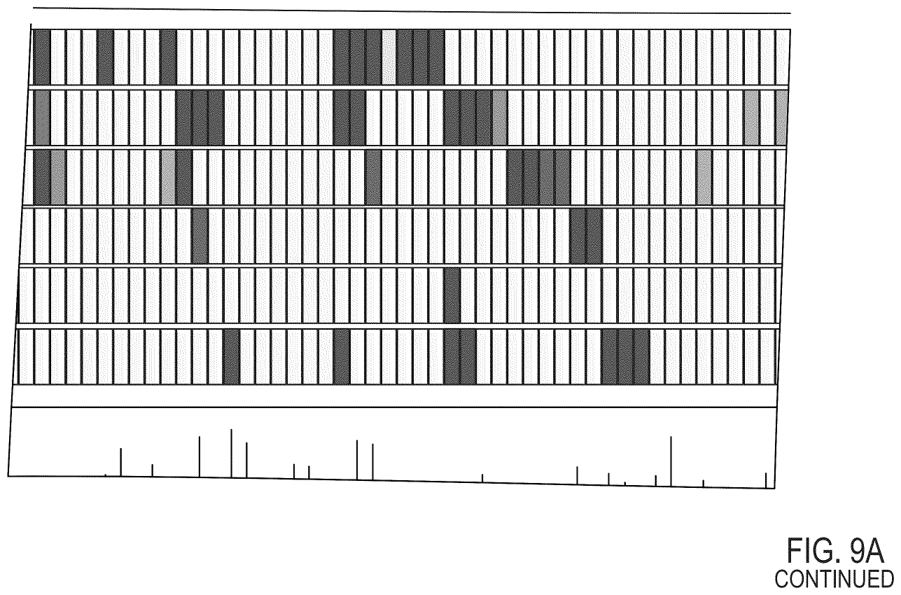

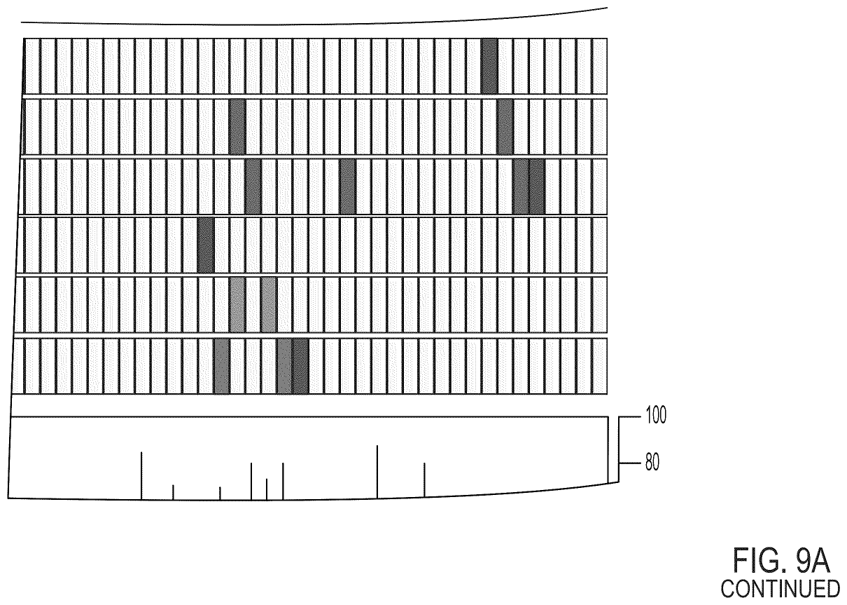

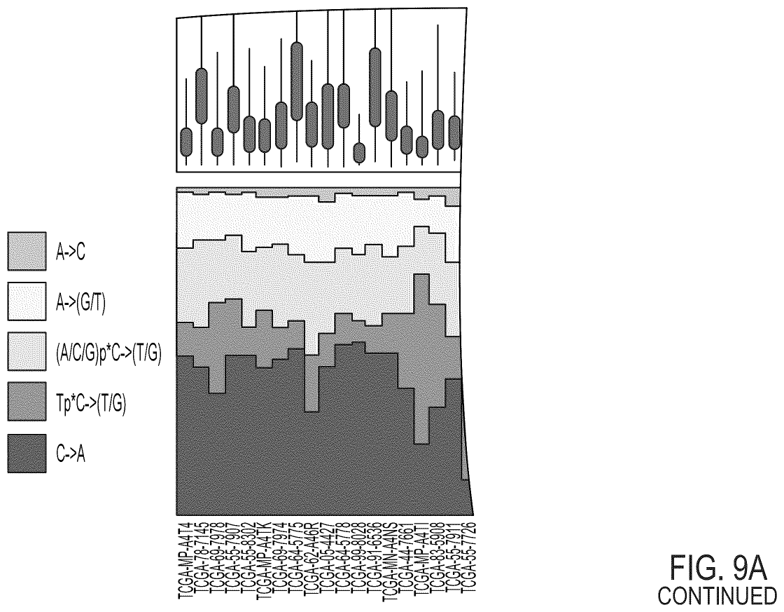

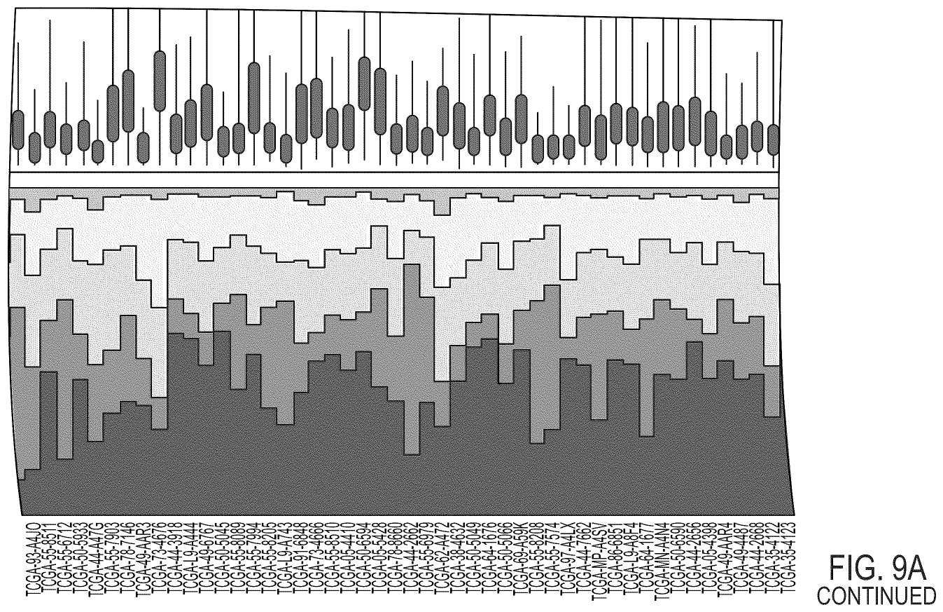

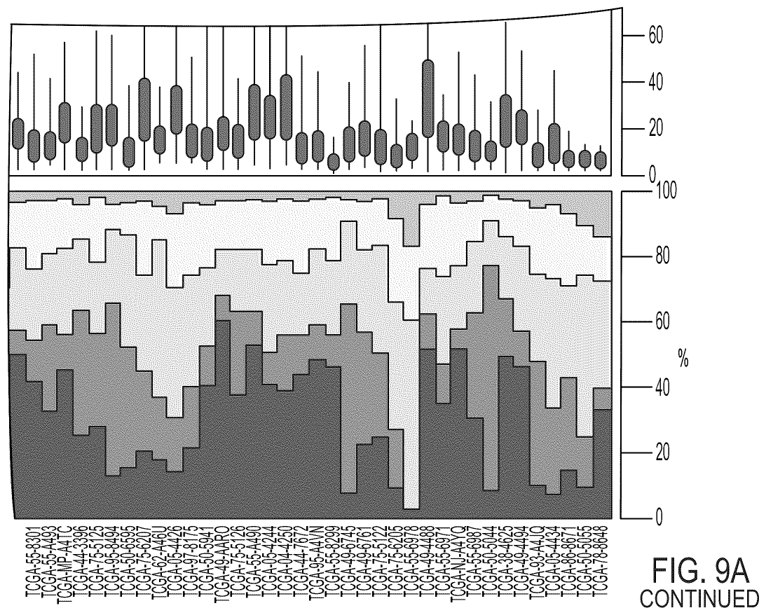

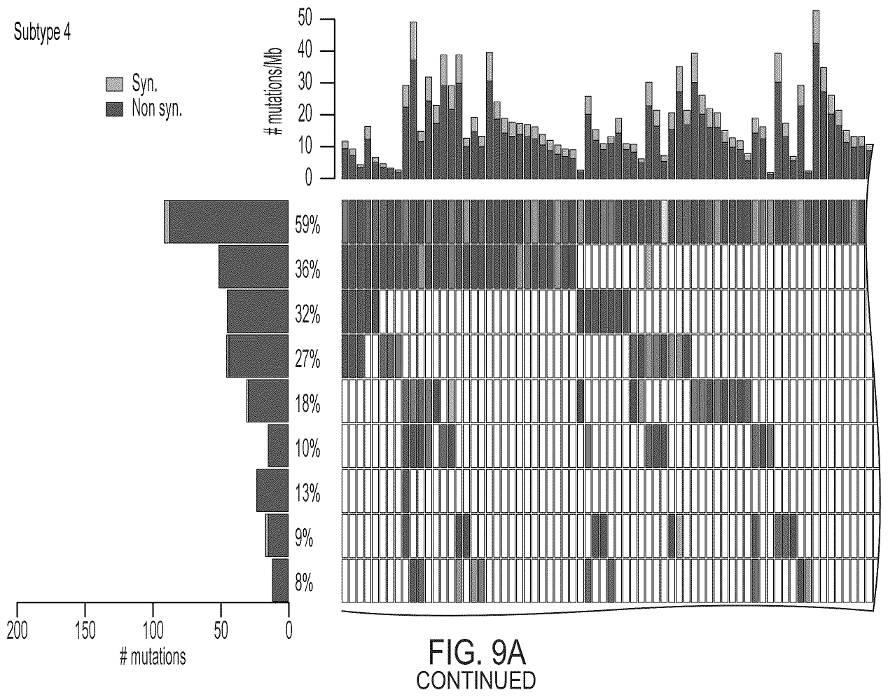

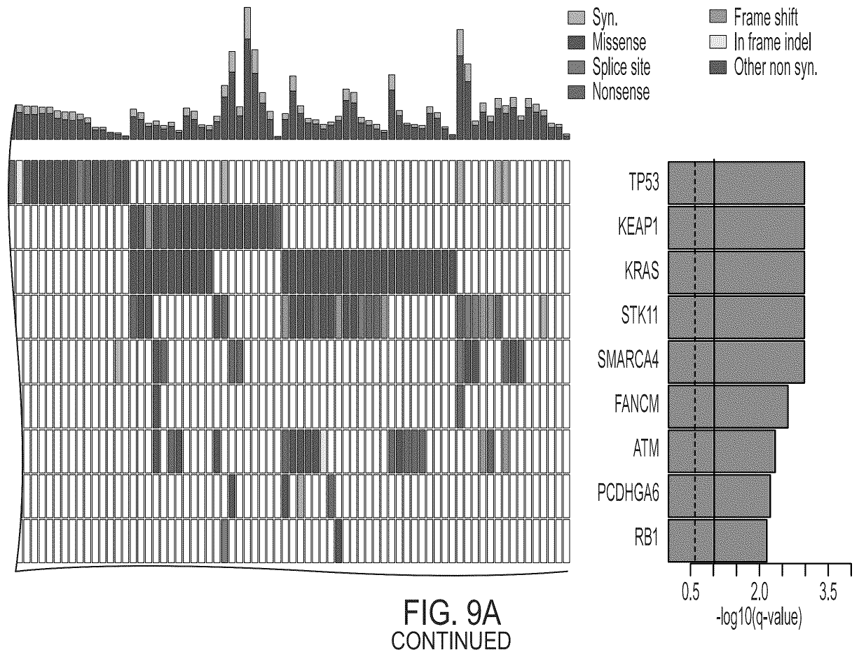

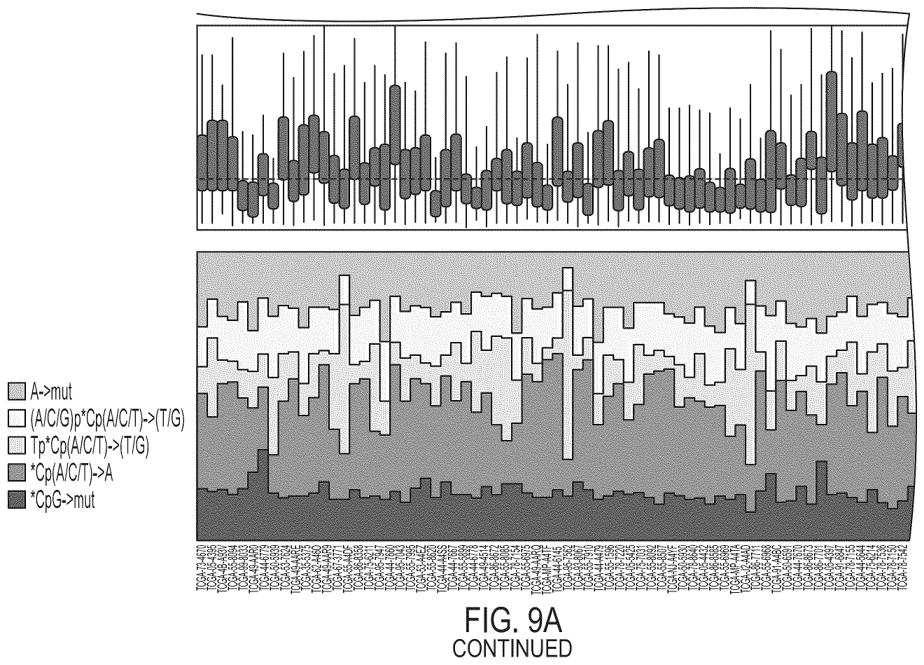

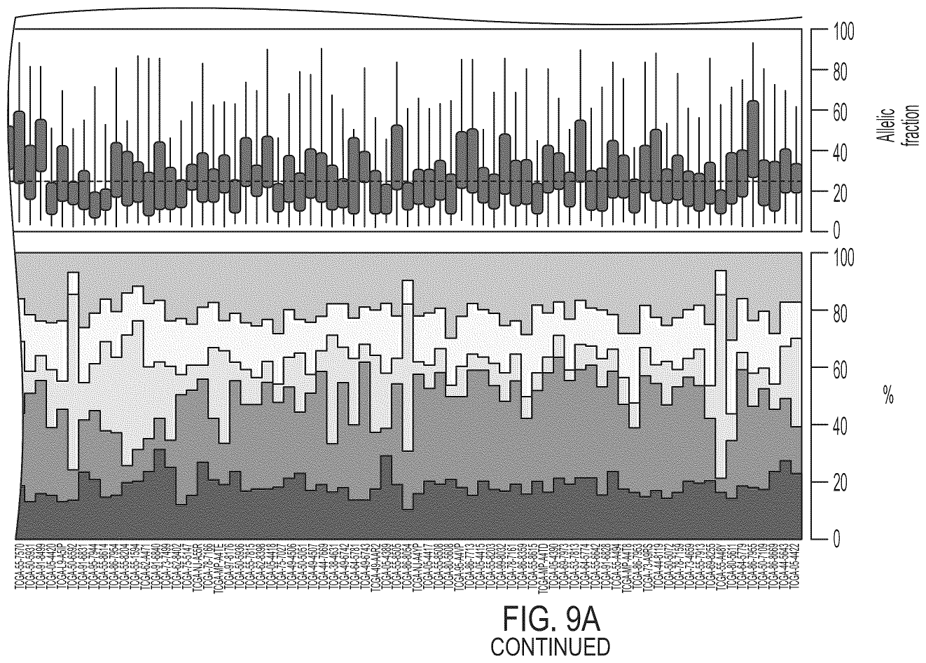

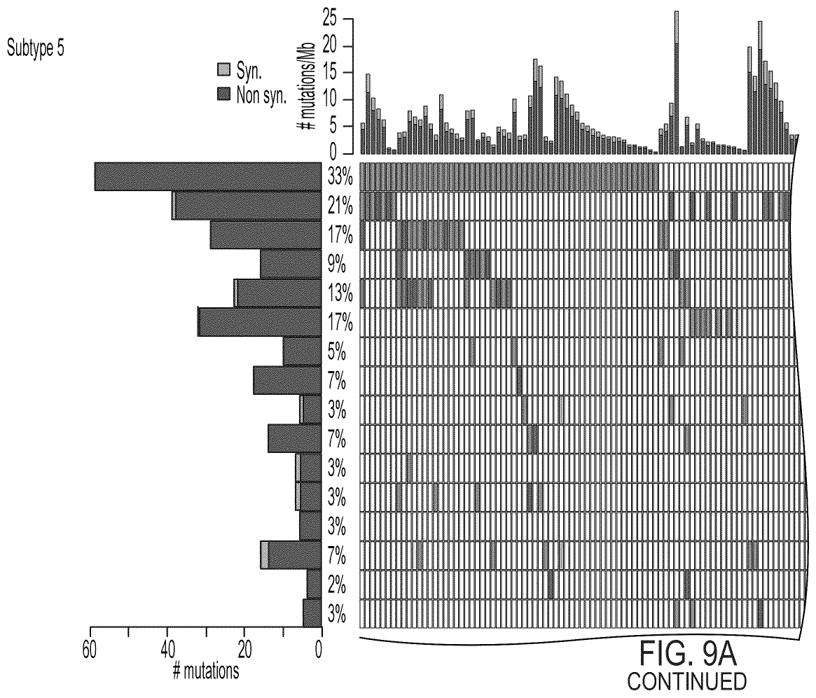

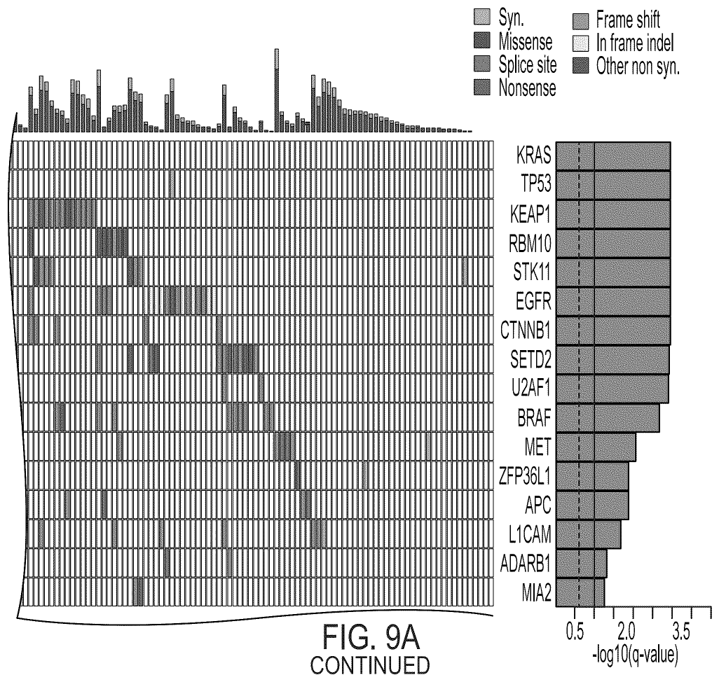

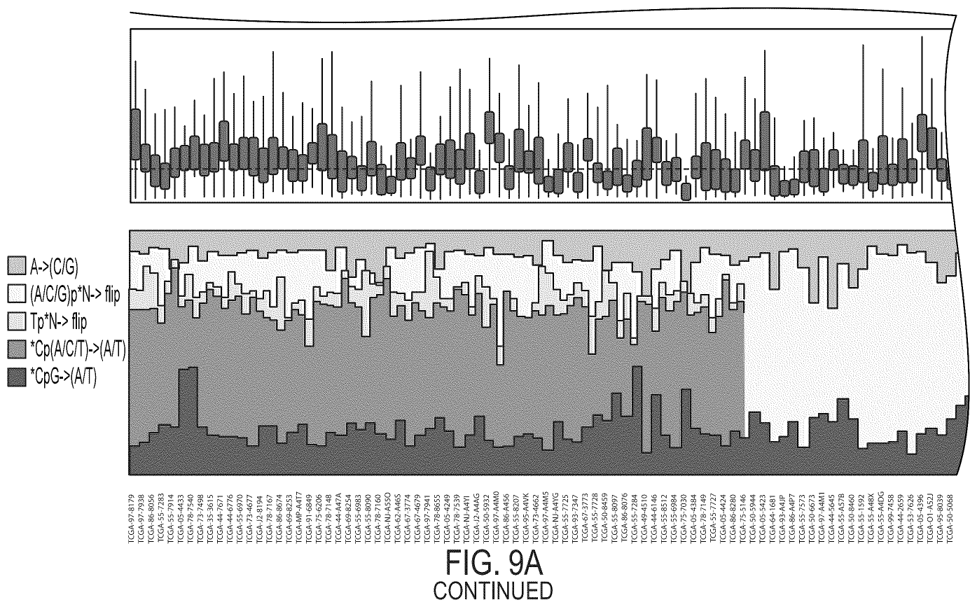

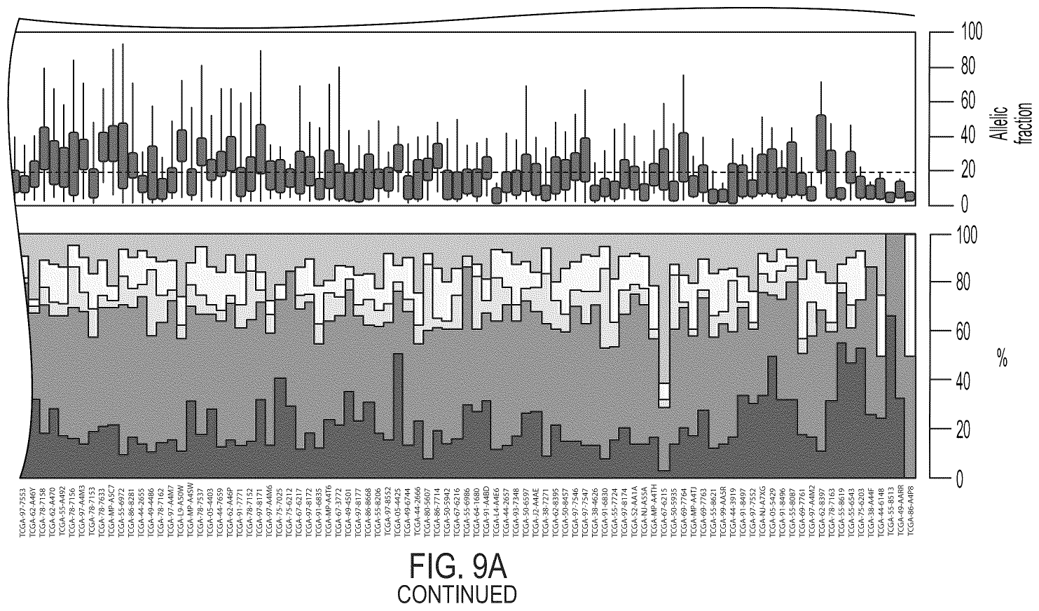

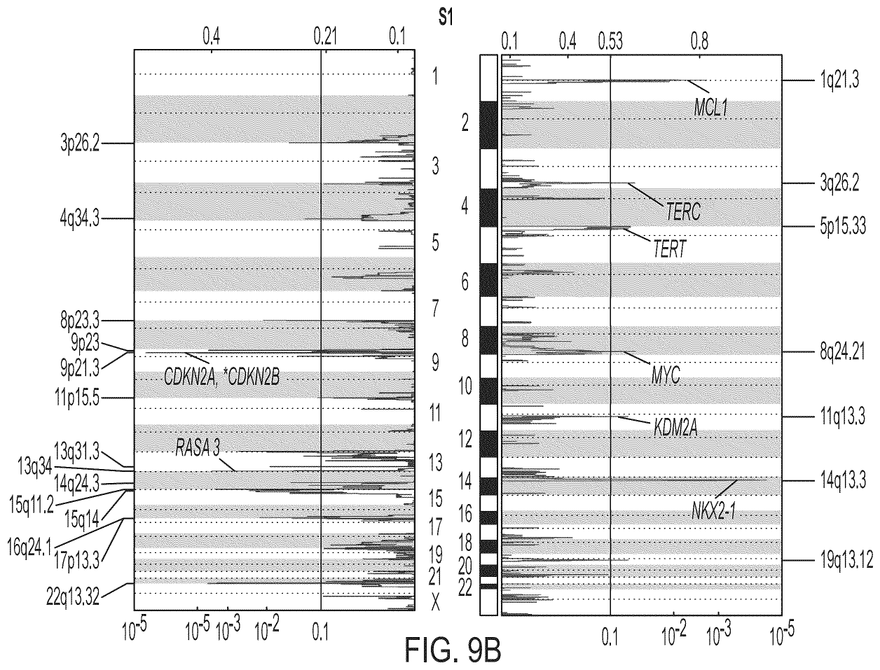

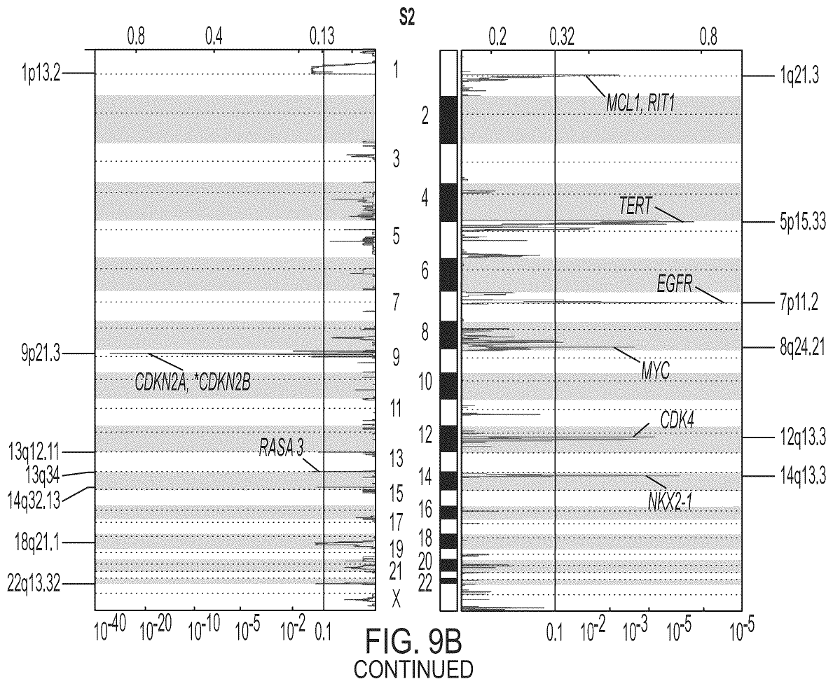

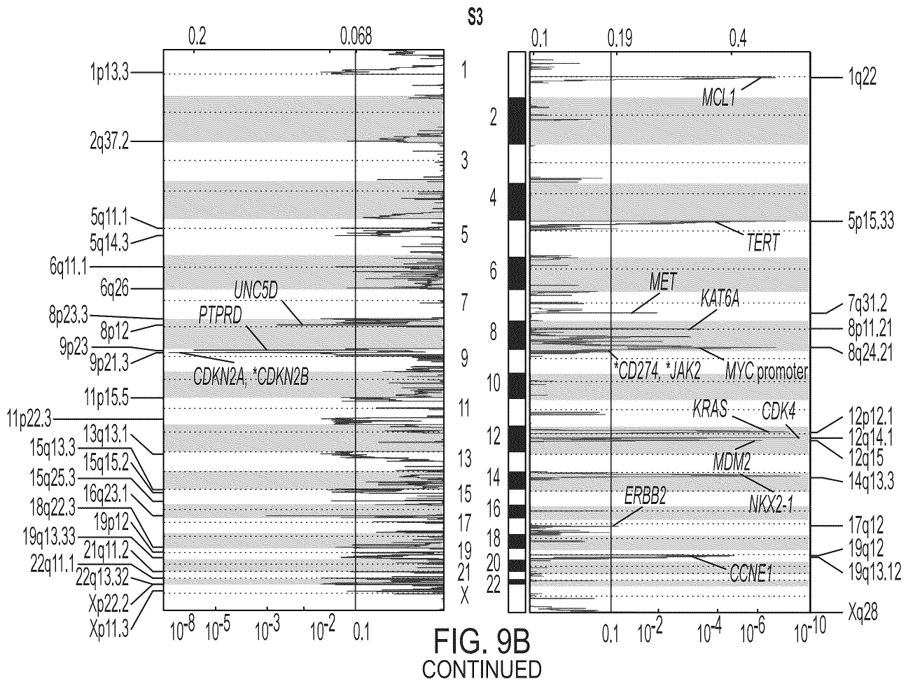

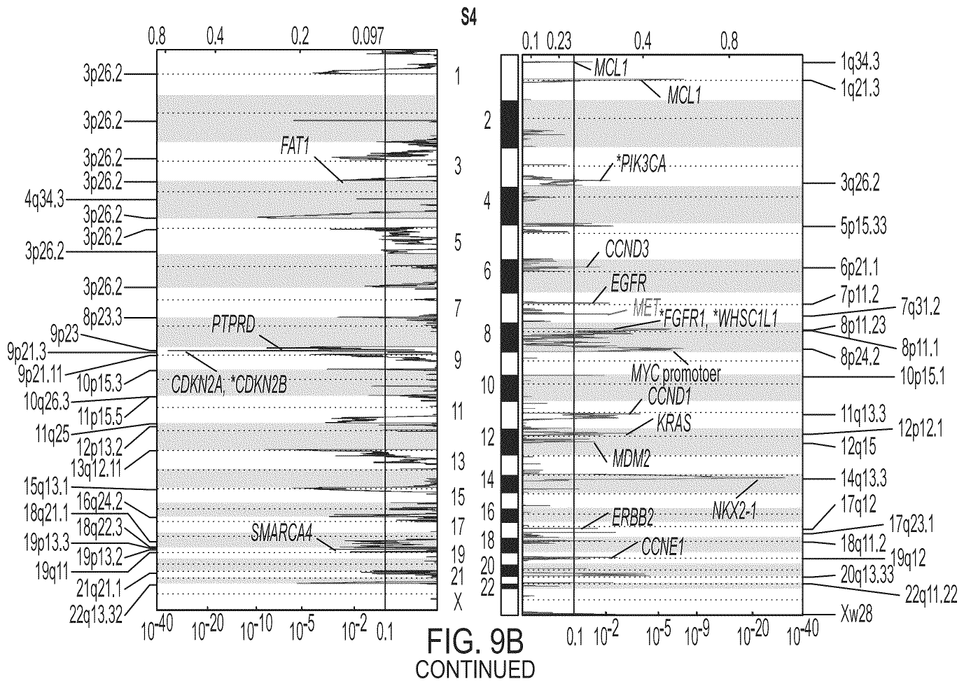

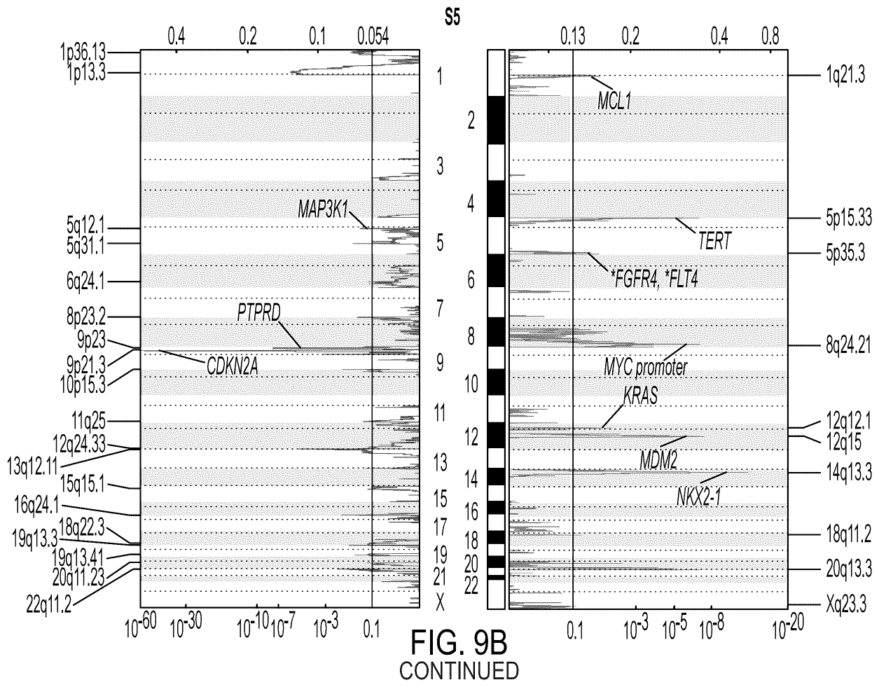

To associate each of the five expression subtypes (S1-S5) with driver events (point mutations, indels, and copy-number alterations), MutSig2CV (Lawrence, Michael S., et al. “Discovery and saturation analysis of cancer genes across 21 tumor types.” Nature 505.7484 (2014): 495-501) and GISTIC 2.0 (Mermel, Craig H., et al. “GISTIC2. 0 facilitates sensitive and confident localization of the targets of focal somatic copy-number alteration in human cancers.” Genome biology 12.4 (2011): R41) were applied to the tumors in each of the subtypes (FIGS. 1D, 9A, and 9B). Multiple subtype-associated drivers were identified in each subtype: EGFR was significantly mutated and amplified in S2 and S5. However, it was altered at a higher frequency in S2 (40% vs 17%). S3 exhibited amplification of the CD274 (PD-L1) gene (near significance: Q value=0.102), MET, and CDK4. The S4 subtype was enriched with activating KRAS and inactivating STK11 mutations. S5 was enriched with EGFR mutations.

The analysis revealed SMARCA4, ATM, FANCM, PCDHGA6 mutations, amplification of MET, FGFR1, and PIK3CA for S4; and BRAF, SETD2, and CTNNB1 mutations in S5. Not intending to be bound by theory, detection of these additional mutations may be due to the larger sample size analyzed herein than in the PP and TRU samples from a previous TCGA study (FIGS. 1D, 9A, and 9B). STK11 mutations in particular were enriched in KRAS-mutant S4 tumors (21 STK11-mutant tumors among 46 KRAS-mutant S4 tumors) (Fisher's exact test P=0.0029), suggesting that S4 tumors might be more resistant to PD-1 inhibitors (Skoulidis et al., 2018).

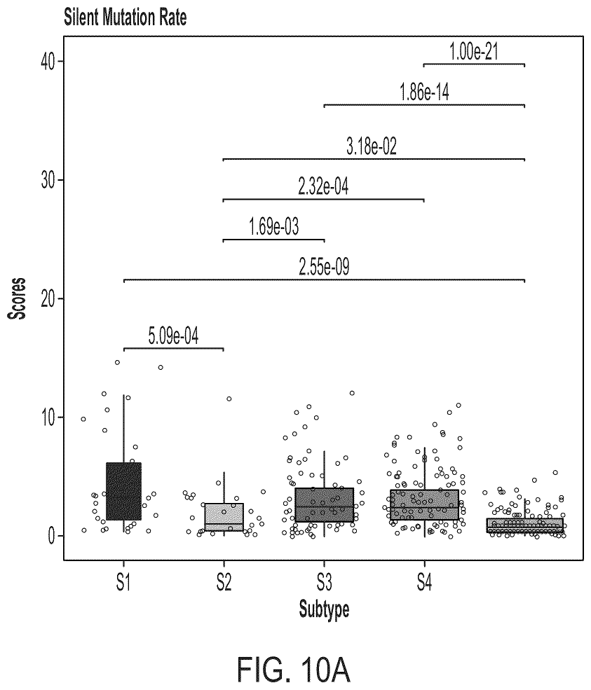

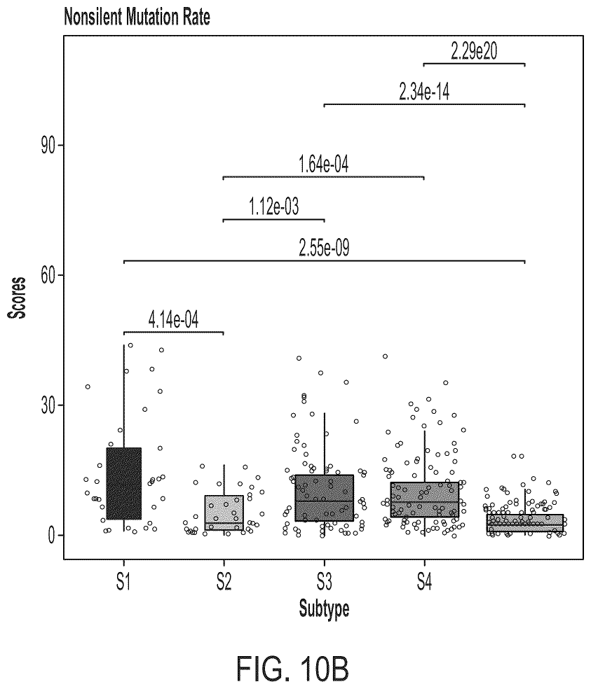

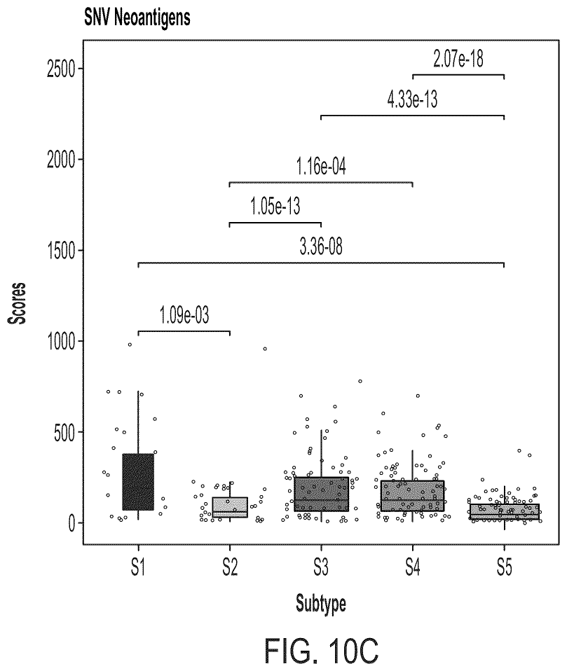

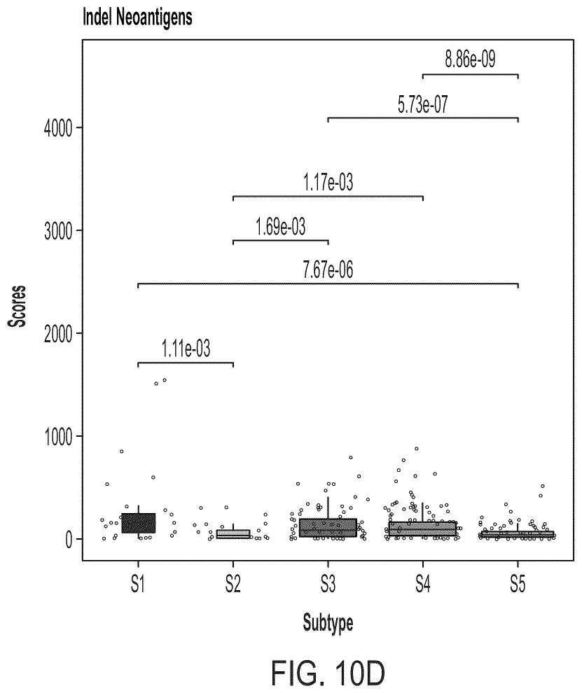

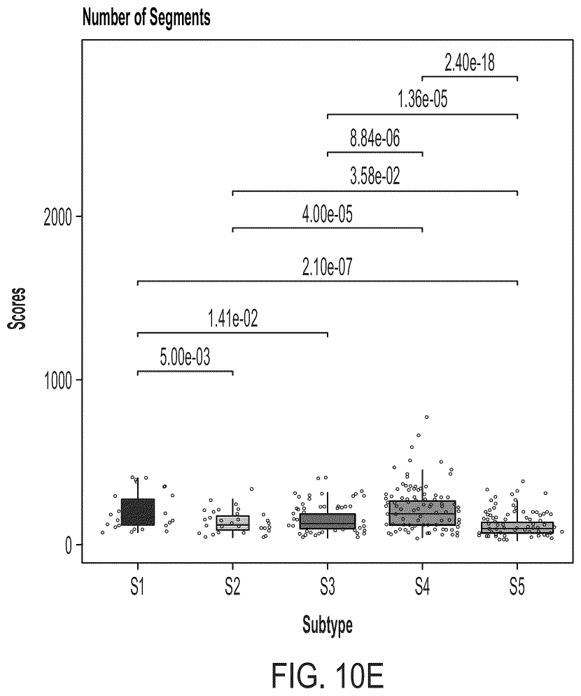

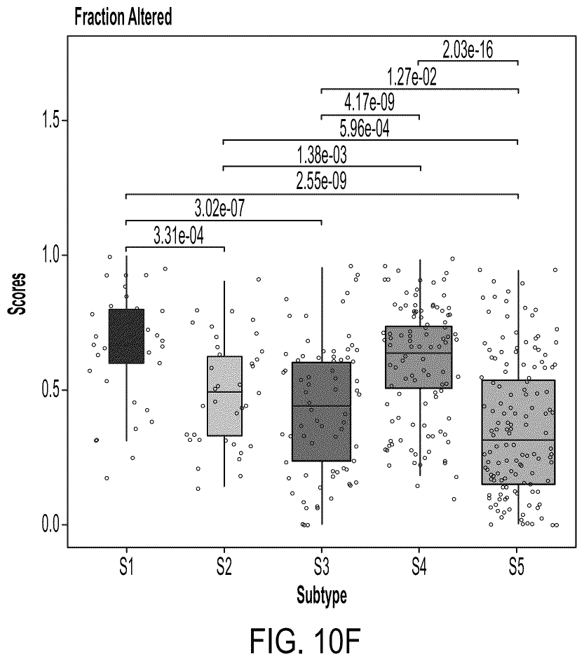

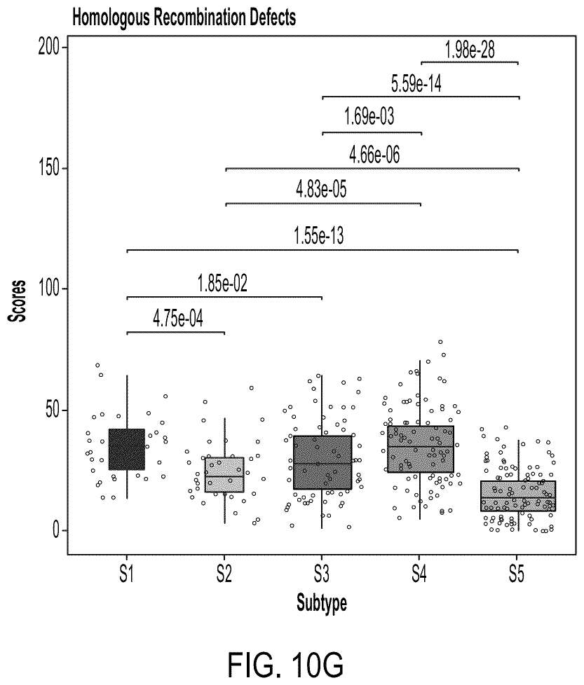

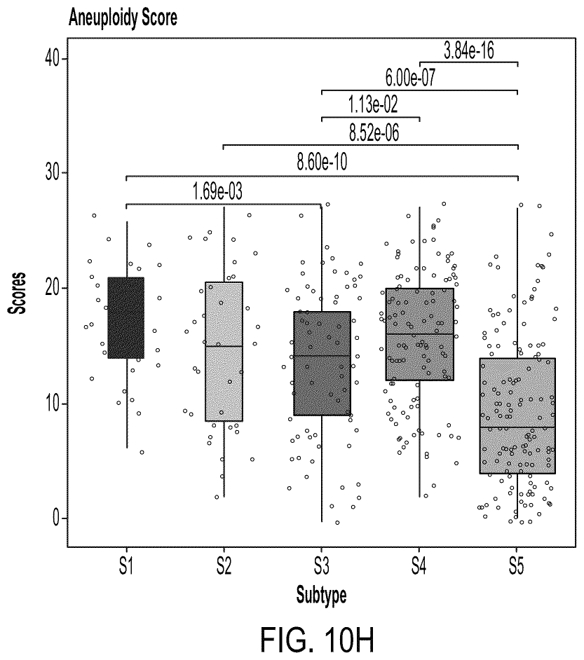

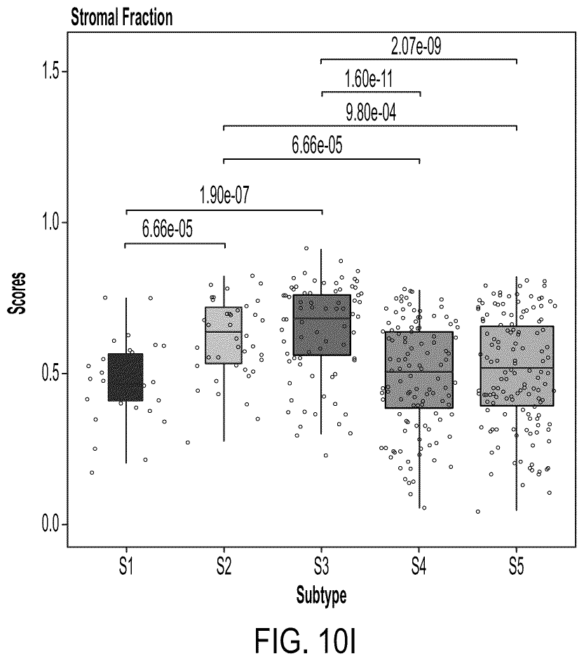

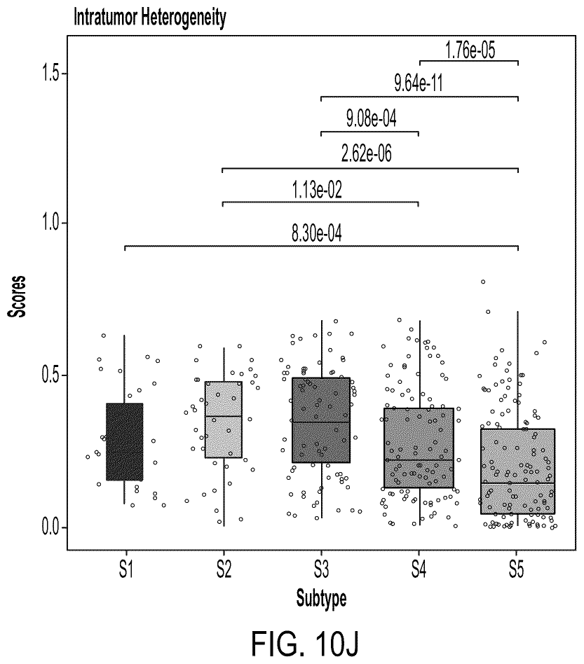

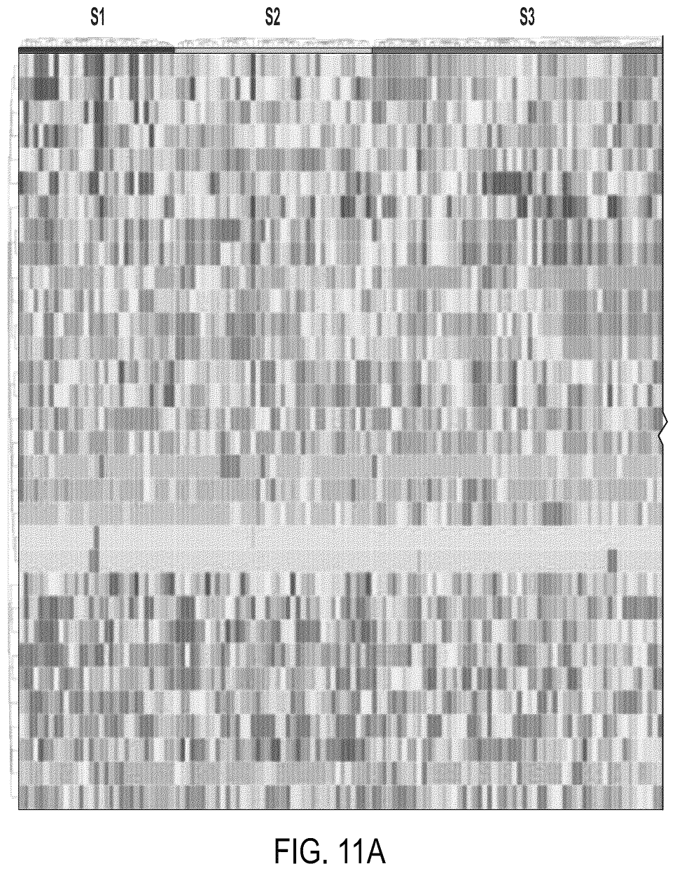

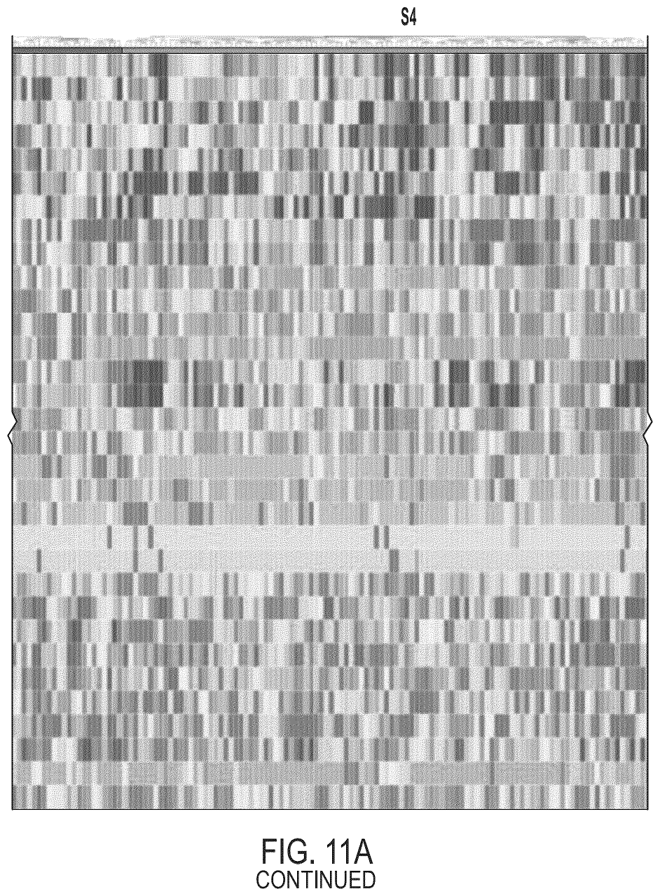

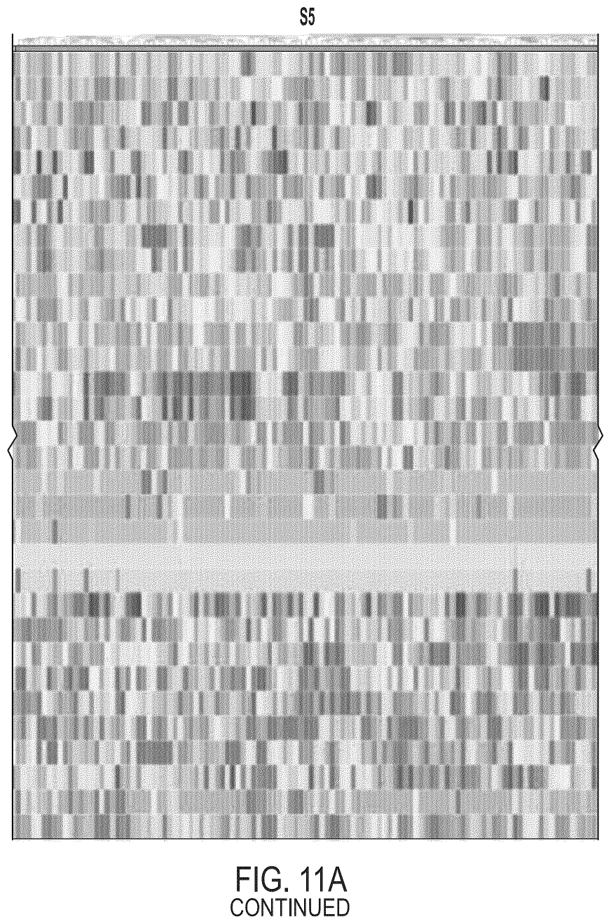

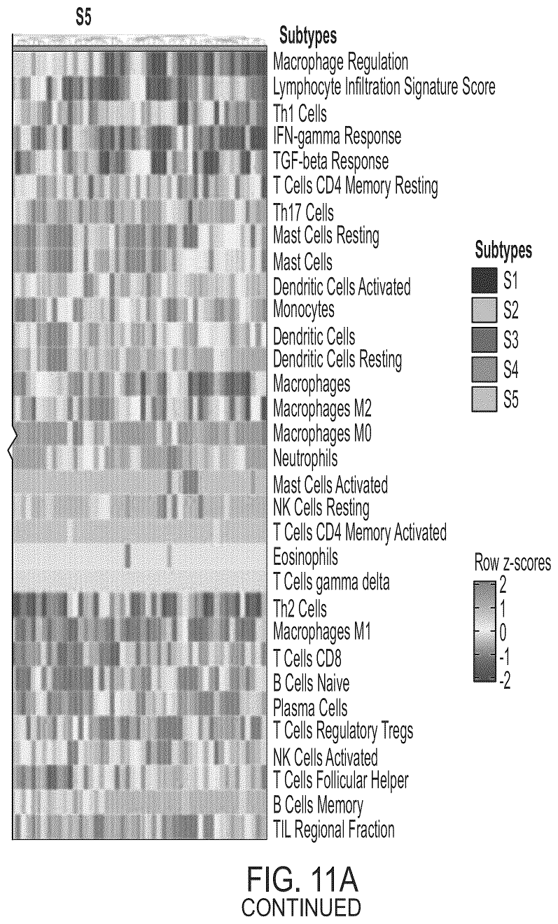

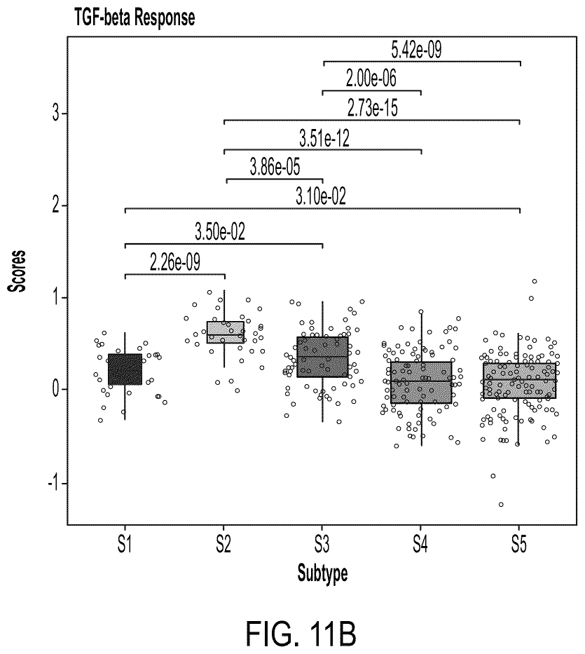

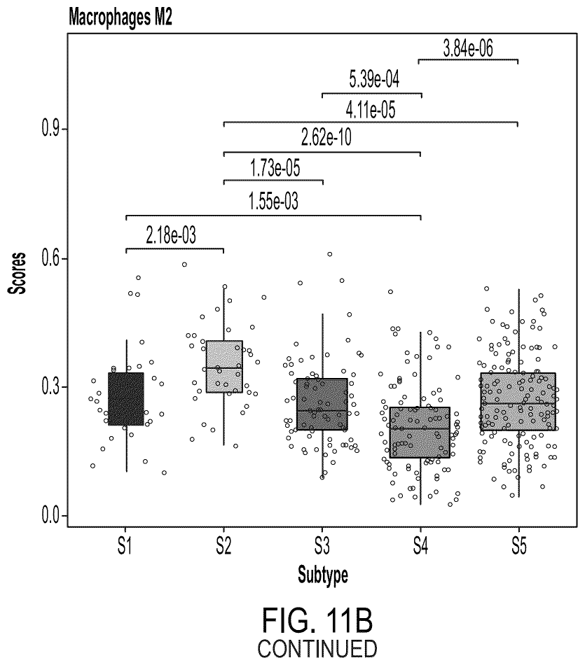

In addition, 16 previously calculated genomic features in TCGA (Thorsson, Vésteinn, et al. “The immune landscape of cancer.” Immunity 48.4 (2018): 812-830) were leveraged to search for differences across the S1-S5 subtypes (FIGS. 10A-10J). It was observed that S2 and S5 had lower somatic tumor mutation burden (TMB) than the other subtypes, including significantly lower frequency of silent and nonsilent mutations as well as significantly fewer indels, which all influenced the number of predicted neoantigens (FIGS. 10A-10D). In features related to somatic copy-number alterations (SCNAs), it was observed that S1 and S4 had significantly higher number of copy-number segments and fraction of genome altered by SCNAs, as well as higher levels of homologous recombination defects and aneuploidy score (FIGS. 10E-10H). These genomic differences provided additional evidence for partitioning the tumors belonging to the prior PI subtype into S1 (higher SCNAs) and S2 (lower TMB) subtypes, with the remaining PI-like tumors falling into the S3 subtype category. Finally, the subtypes were associated with immune cell populations that were derived by deconvolving expression data in Thorsson et al. (Thorsson, Vésteinn, et al. “The immune landscape of cancer.” Immunity 48.4 (2018): 812-830) (FIG. 11A). It was found that S2 showed significantly higher TGF-beta levels and a higher fraction of M2 macrophages (FIG. 11B), which, while not intending to be bound by theory, may be explained by secretion of TGF-beta by M2 macrophages to promote immune suppression in S2. Collectively, the genomic characterization of the five expression subtypes (S1-S5) showed that each subtype has a distinct biology.

It was next asked whether the expression subtypes were associated with disease prognosis and a significant association was found between the subtypes and disease-specific survival (DSS) (P value=0.046). The S5, which showed enrichment with the TRU subtype, showed the best prognosis (FIG. 8E).

### Example 2: Subtype-Specific Cancer Vulnerabilities

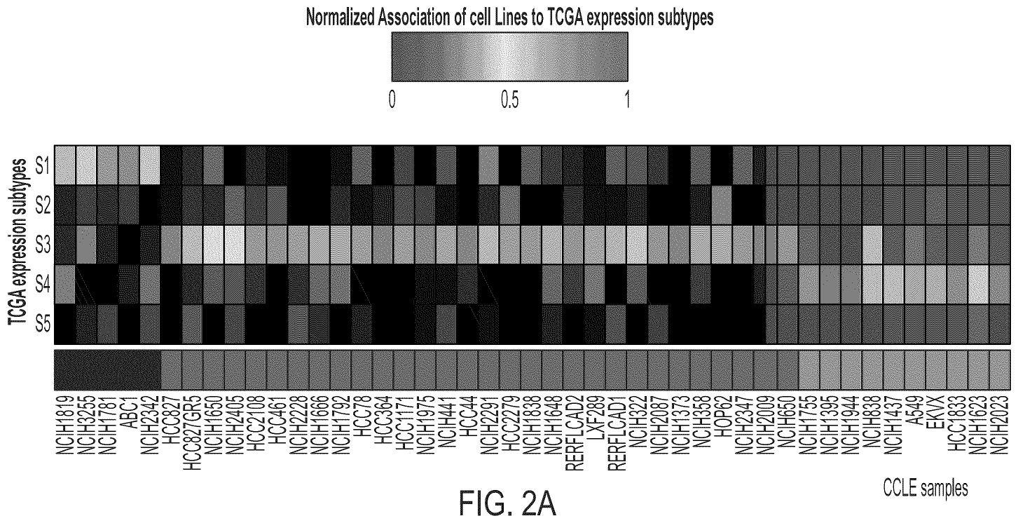

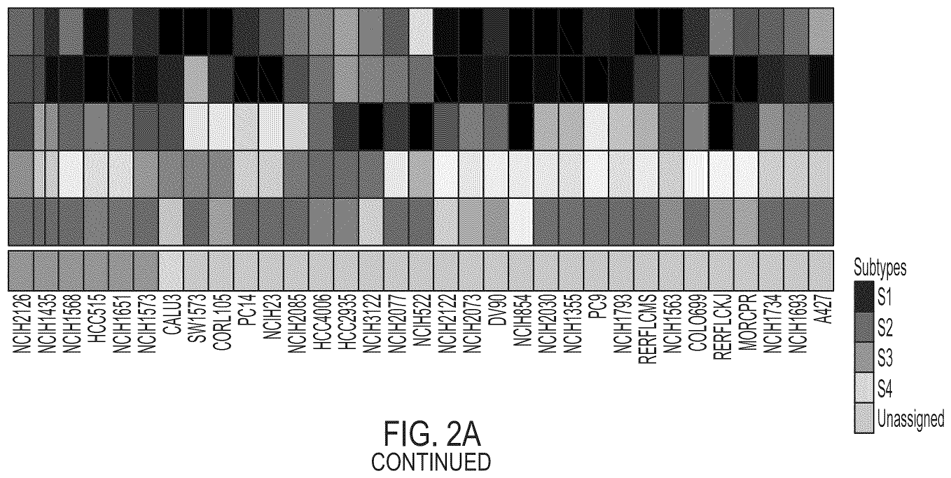

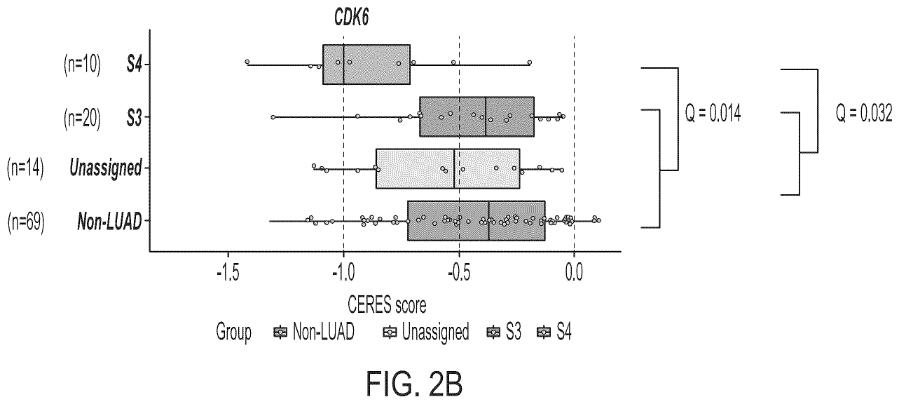

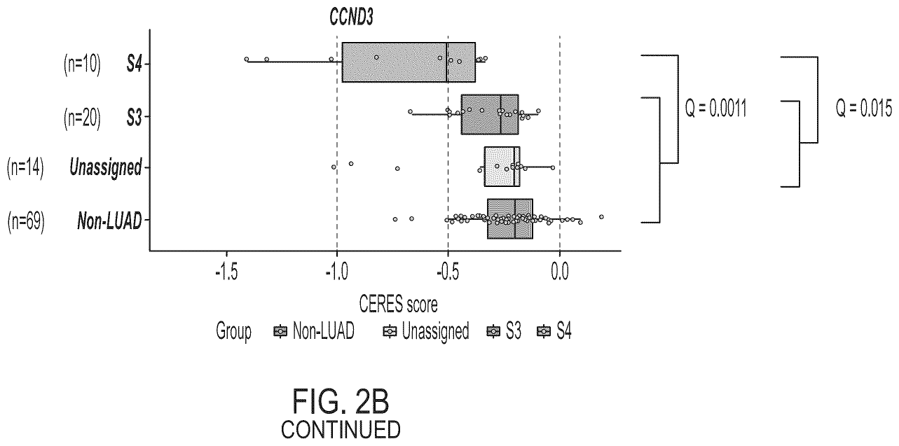

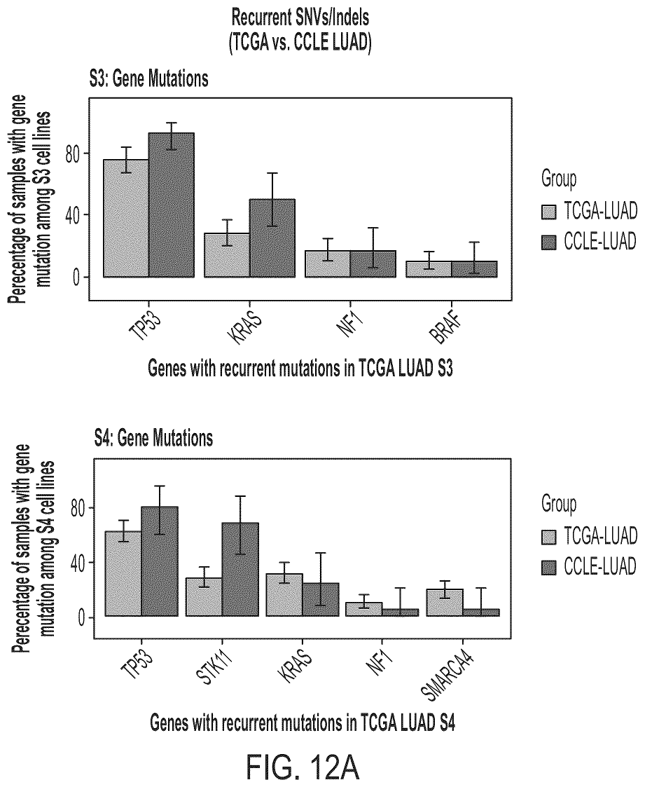

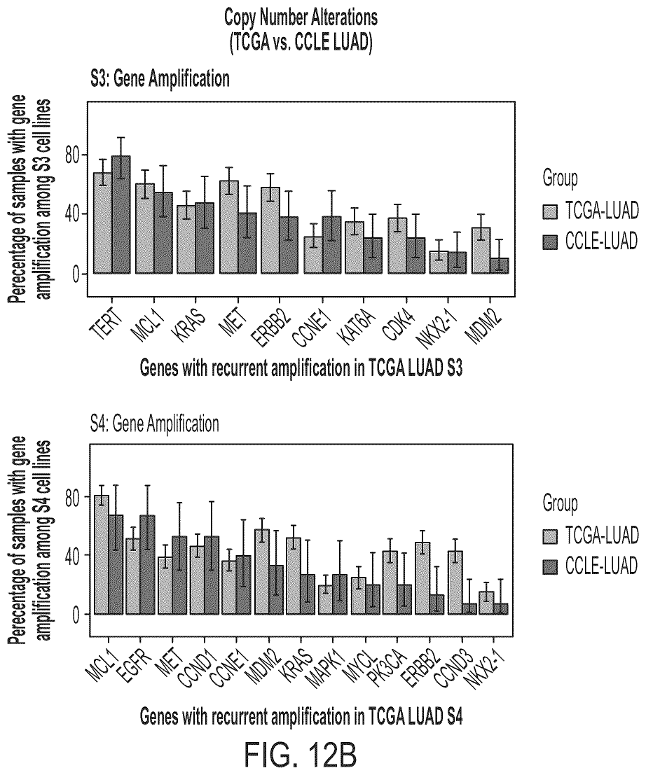

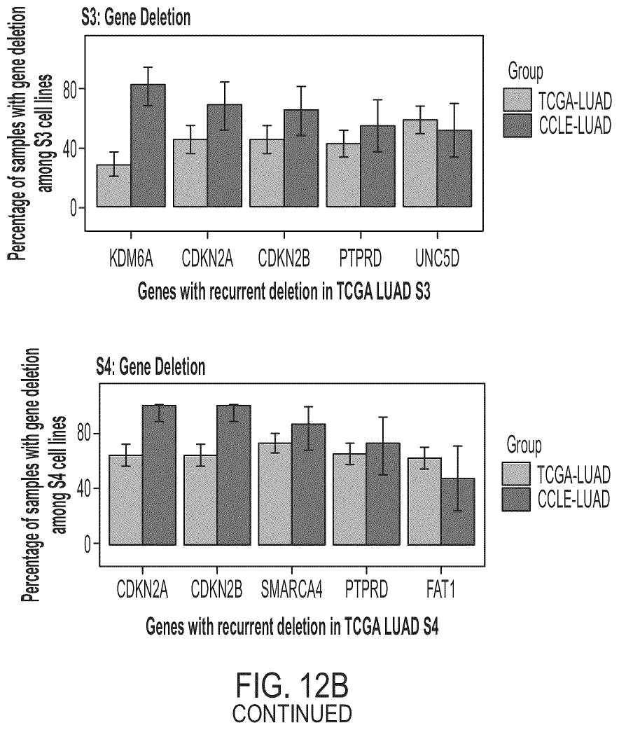

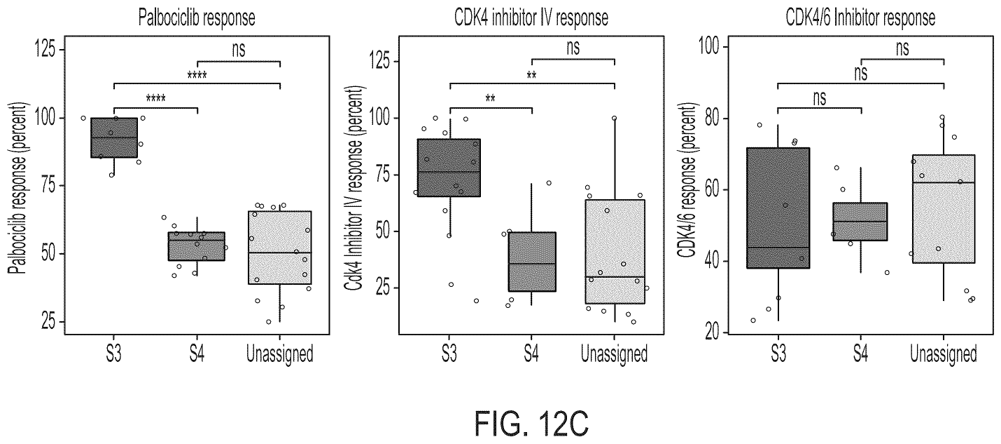

It was next asked whether the Cancer Cell Line Encyclopedia (CCLE) (Barretina, Jordi, et al. “The Cancer Cell Line Encyclopedia enables predictive modelling of anticancer drug sensitivity.” Nature 483.7391 (2012): 603-607; and Ghandi, Mahmoud, et al. “Next-generation characterization of the cancer cell line encyclopedia.” Nature 569.7757 (2019): 503-508) and DepMap (Tshemiak, Aviad, et al. “Defining a cancer dependency map.” Cell 170.3 (2017): 564-576) resources, which provide expression data as well as CRISPR and drug screening data for ˜1,100 cell lines, could be leveraged to find subtype-specific cancer vulnerabilities. The 78 Cancer Cell Line Encyclopedia (CCLE) lung adenocarcinoma (LUAD) cell lines were first probabilistically classified with expression data into the lung adenocarcinoma (LUAD) expression subtypes (45 Cancer Cell Line Encyclopedia (CCLE) lung adenocarcinoma (LUAD) cell lines with both expression and CRISPR data available) using subtype-specific marker genes (FIGS. 1A, 2A, and 8F and Table 15). Since only S3 (n=31) and S4 (n=16) had a sufficient number of samples for downstream analysis, these subtypes were selected for further analysis. To validate the subtype classification, it was confirmed that the S3- and S4-associated cell lines harbored genetic events, somatic point mutations and copy-number alterations (FIGS. 12A and 12B) that were consistent with patients associated with S3 and S4. The cancer vulnerabilities of 21 lung adenocarcinoma (LUAD) driver oncogenes between S3/S4 and the other Cancer Cell Line Encyclopedia (CCLE) lung adenocarcinoma (LUAD) cell lines were then compared (FIG. 2B). Two significant vulnerabilities were identified for S4: CDK6 and the CDK6-cyclin D3 complex gene, CCND3 (significant both within the 45 lung adenocarcinoma (LUAD) cell lines and all 114 lung cancer cell lines with both CRISPR and expression data available). This finding suggested that S4 tumors may be dependent on the CDK6 pathway and thus potentially vulnerable to CDK6 inhibition.

CDK4 was nominally significant (P=0.01; Q=0.13) as a vulnerability for S3, which was consistent with the recurrent genomic alterations in the CDK4 (FIGS. 9B and 12B) pathway in S3. Therefore, the sensitivity of S3-associated cell lines to CDK4 specific inhibition was tested using two CDK4 inhibitors: Palbociclib and CDK4/6 Inhibitor IV (CAS359886-84-3, a triaminopyrimidine compound that acts as a reversible and ATP-competitive inhibitor; termed CINK4). Both compounds are known CDK4/6 inhibitors at high concentrations; however, at low concentrations, they are potent CDK4-only inhibitors that induce G1 cell cycle arrest and senescence in retinoblastoma protein (Rb)-proficient cell lines. Nine cell lines were treated with either palbociclib or CINK4—4 from the S3 subtype (HCC78, HCC827, NCIH1975, NCIH1838), 3 from the S4 subtype (NCIH1395, NCIH1833, NCIH1755) and two that were not assigned to any subtype (ABC1, CALU3)—and proliferation was measured with and without drug. As expected, the S3 cell lines showed significantly lower proliferation (higher response) compared to the S4 and unassigned cell lines (Palbociclib: P=1.6×10⁻⁵ and P=4.1.×10⁻⁶ respectively (FIG. 12C left panel and CINK4: P=3.5×10⁻³ and P=3.3×10⁻³; FIG. 12C middle panel). Not intending to be bound by theory, these results demonstrated that the S3 subtype had dependency on CDK4, suggesting that a combined therapy that includes a CDK4 inhibitor may be beneficial in such patients. Since palbociclib inhibits both CDK4 and CDK6 at higher concentrations, CDK6-only inhibition could not be tested in S4 cell lines using palbociclib. Higher doses of palbociclib inhibited proliferation in all cell lines (FIG. 12C, left panel). Taken together, the CRISPR data and drug sensitivity experiments demonstrated specific vulnerabilities for CDK4 in S3 and CDK6/CCND3 in S4 subtypes.

### Example 3: Proteogenomic Analysis Reveals Distinct Protein Regulation Between S3 and S4

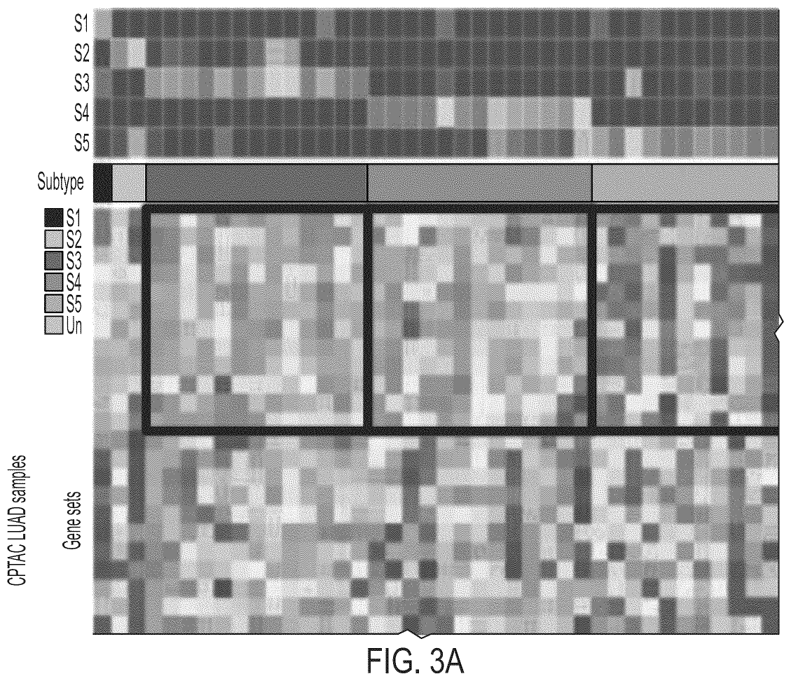

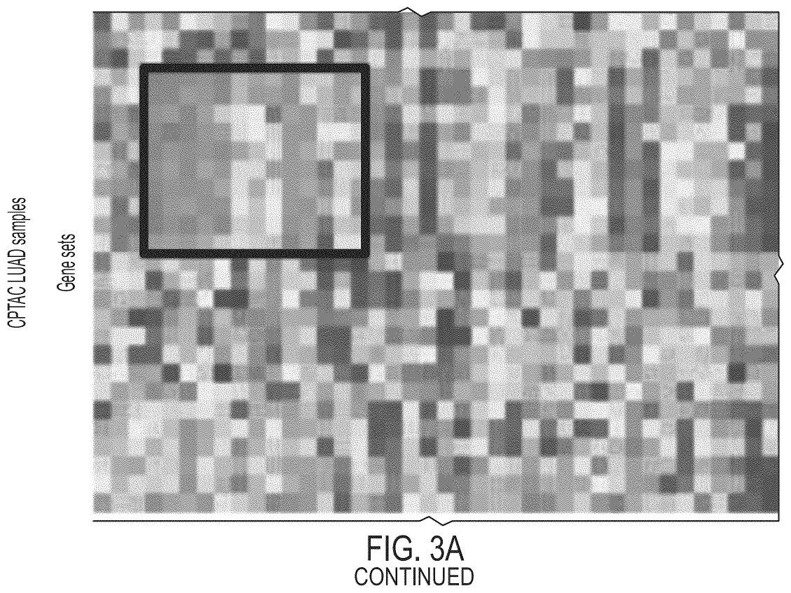

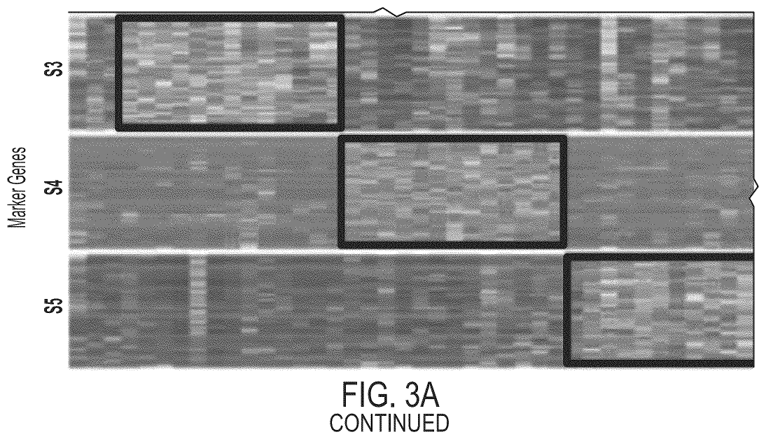

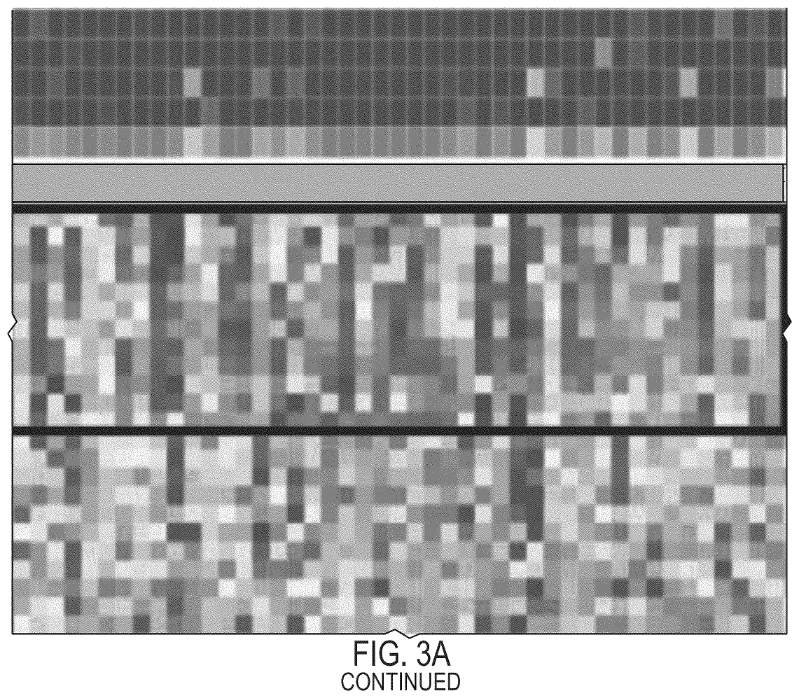

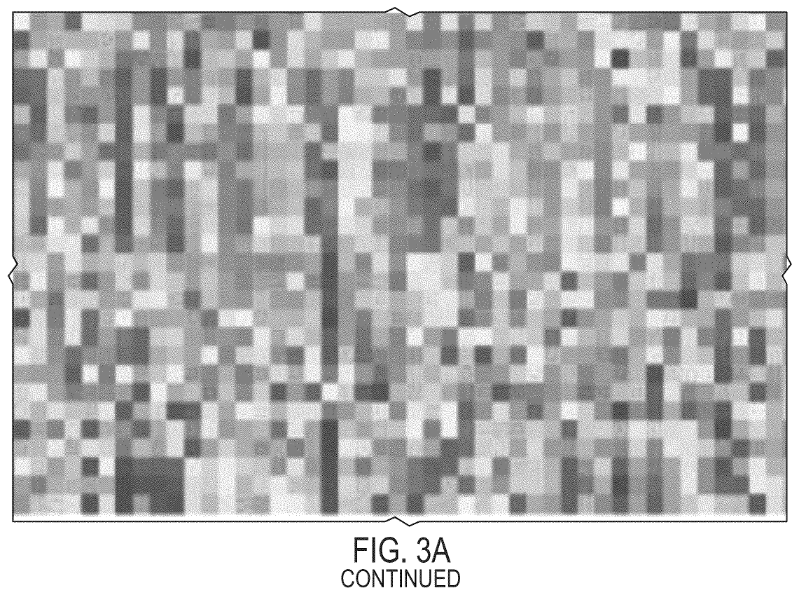

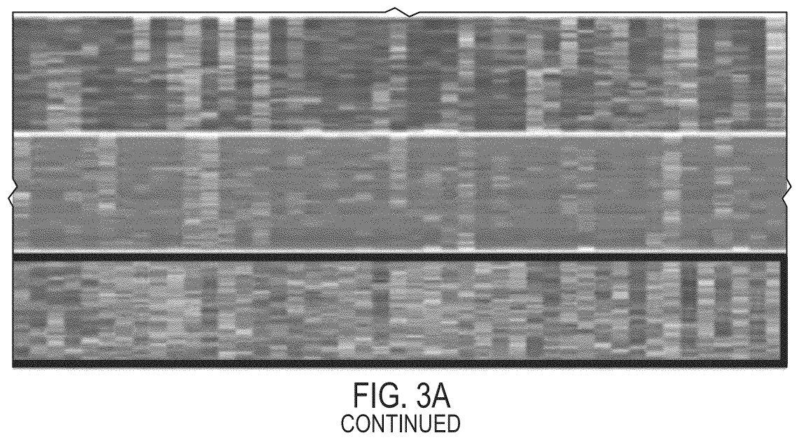

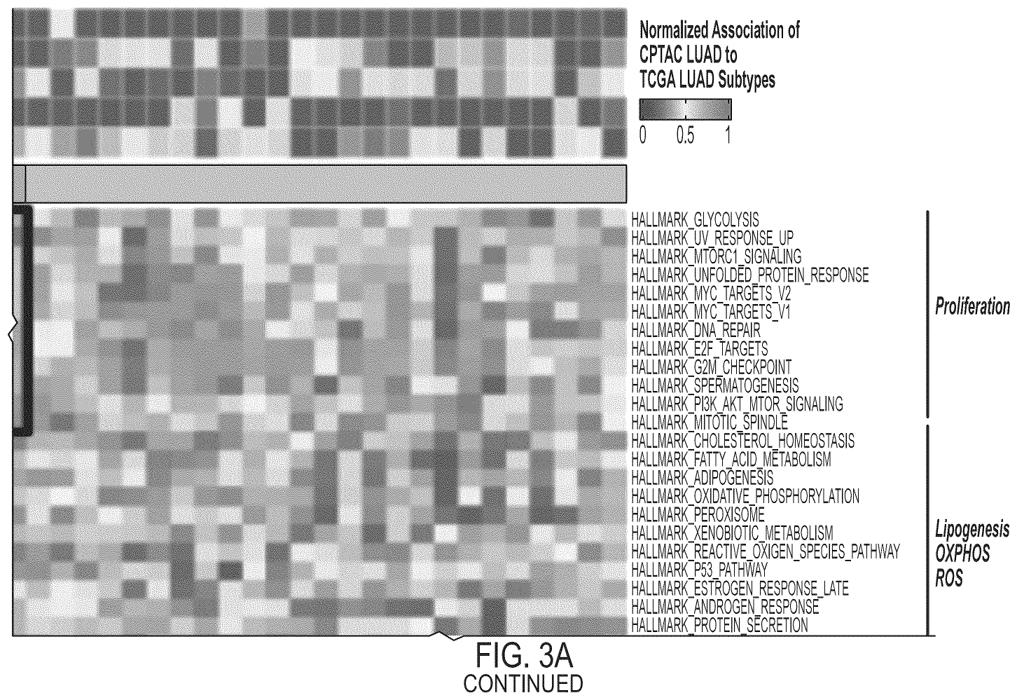

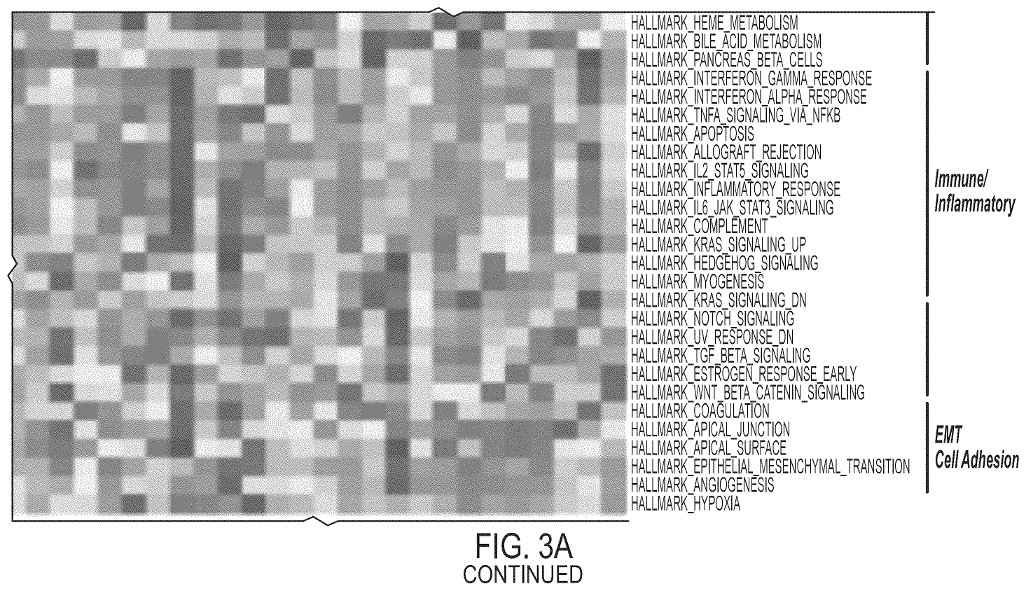

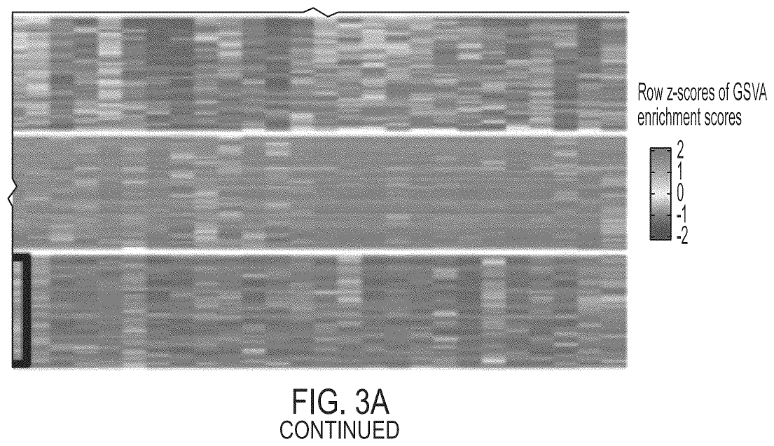

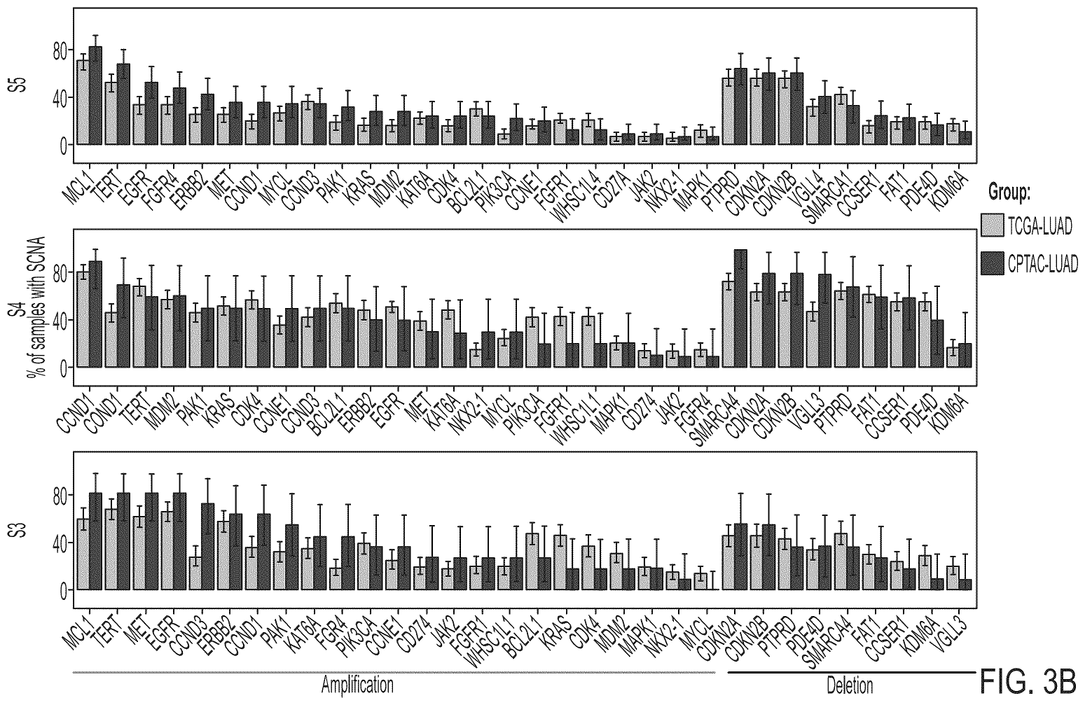

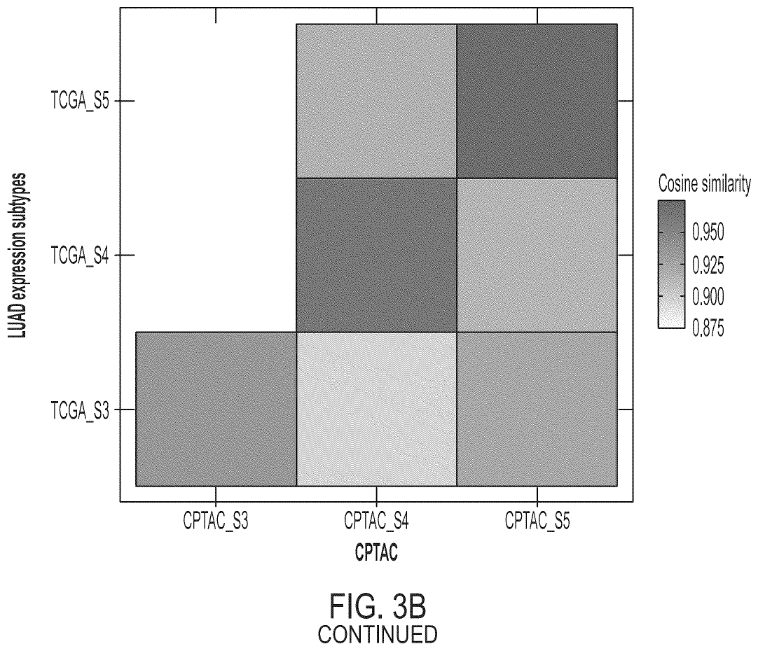

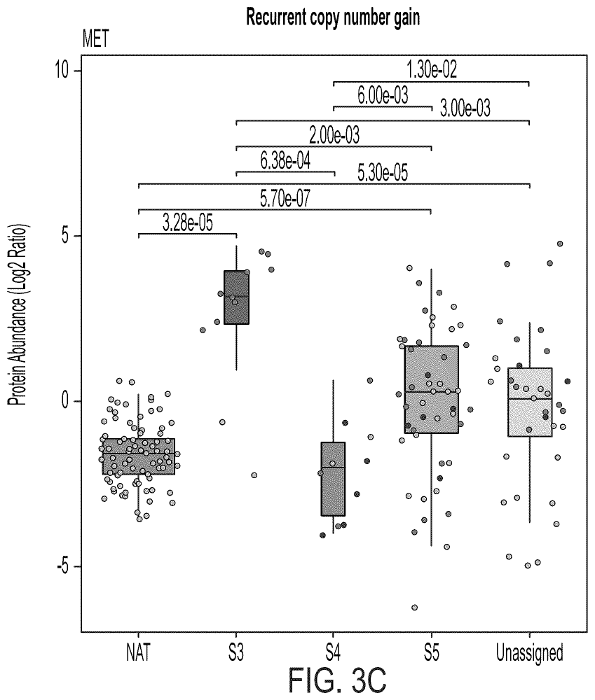

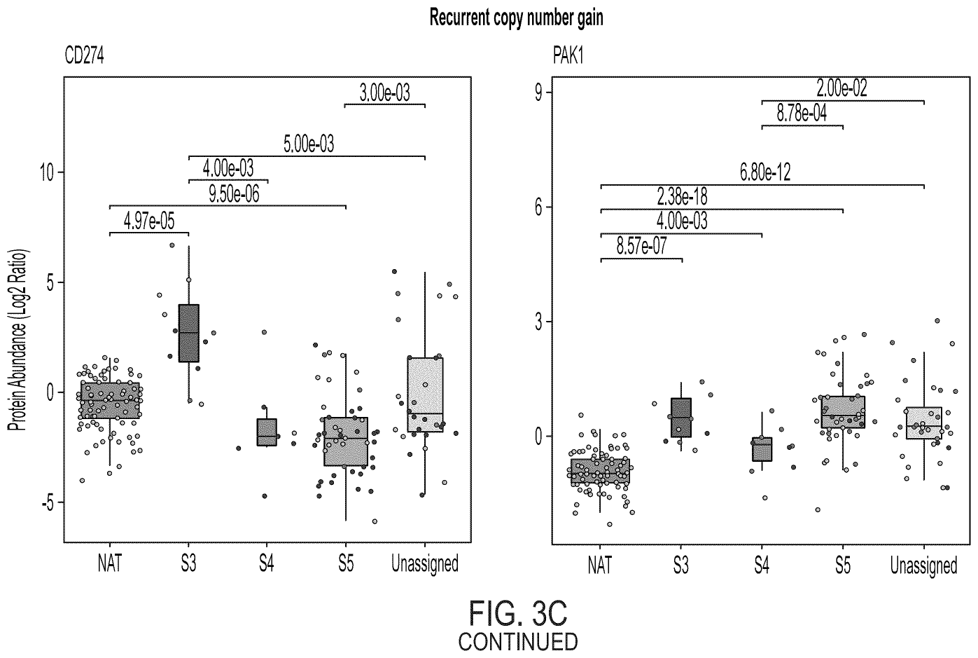

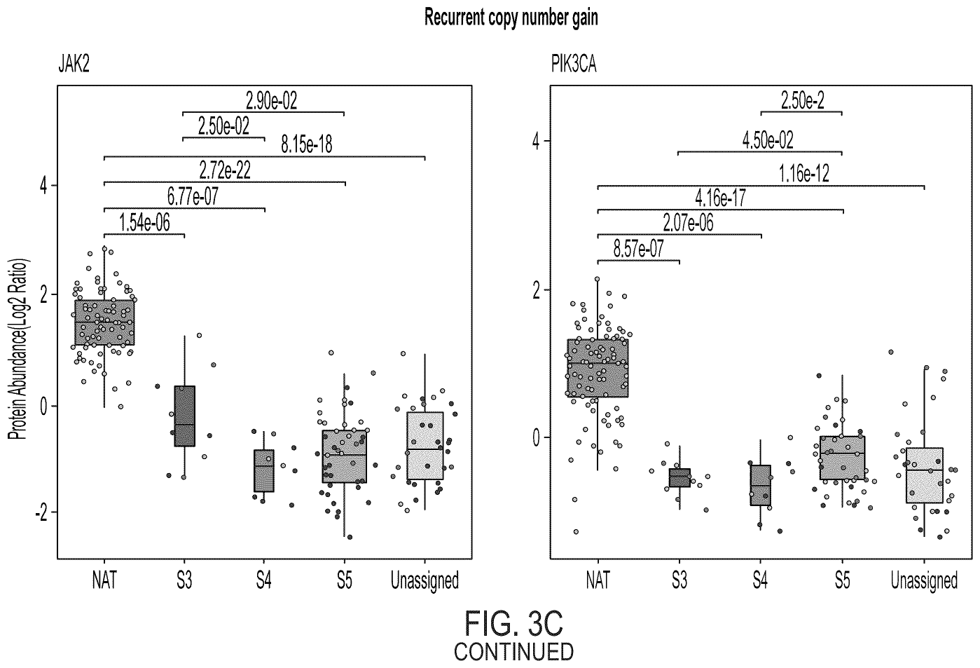

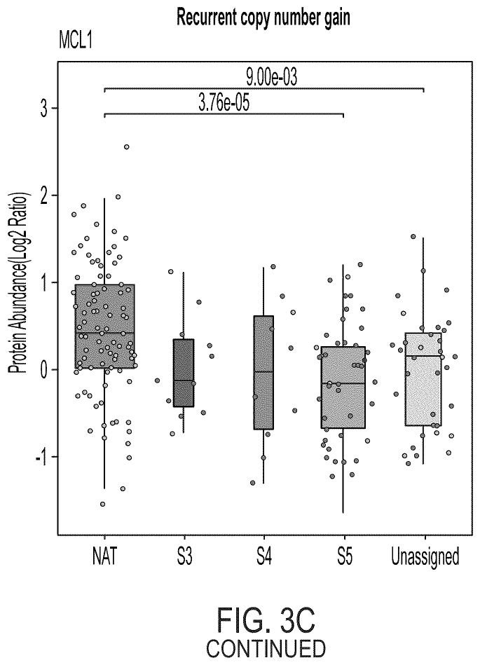

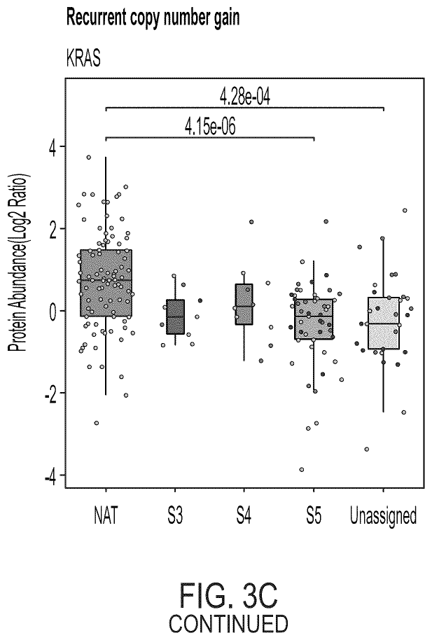

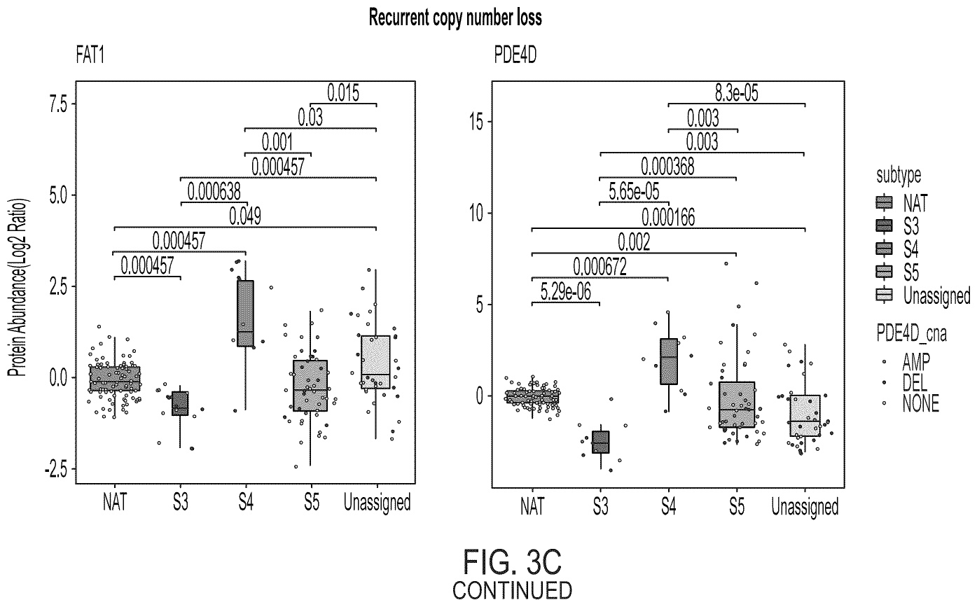

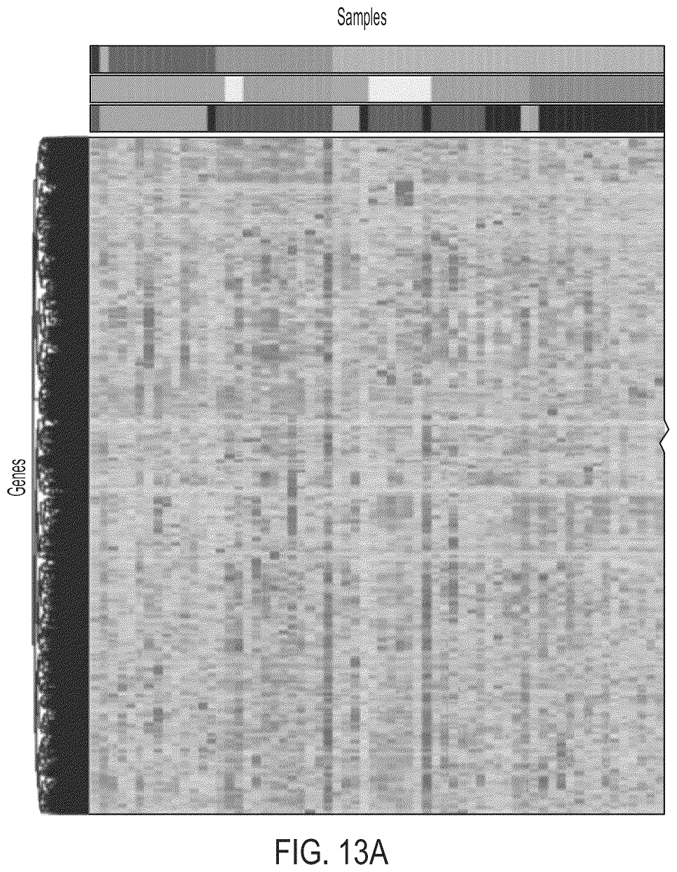

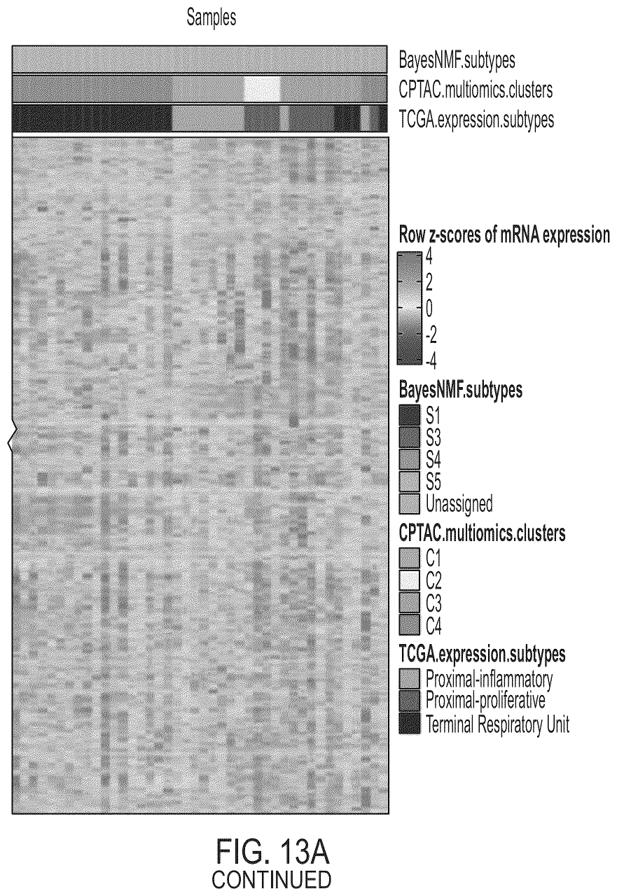

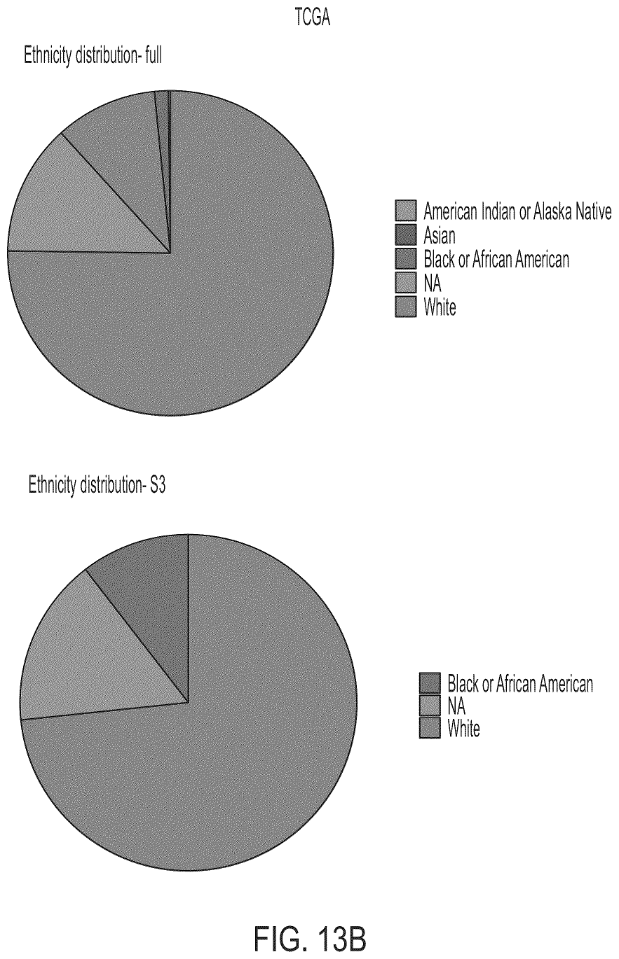

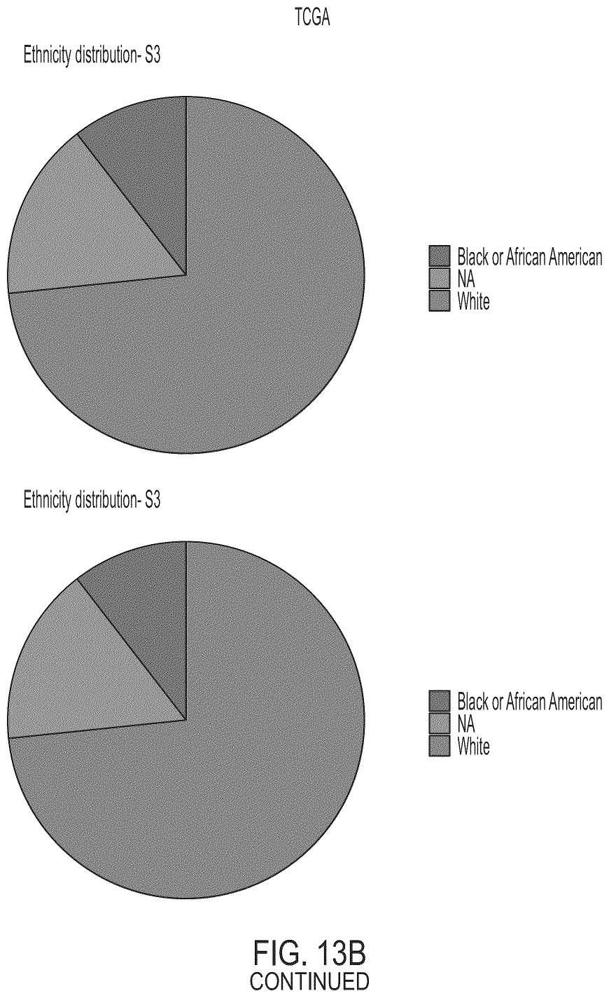

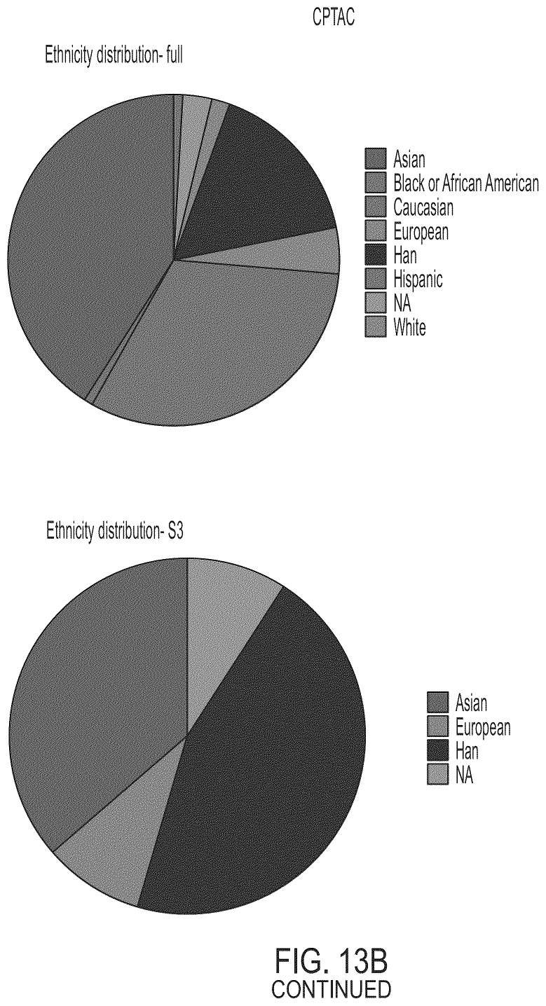

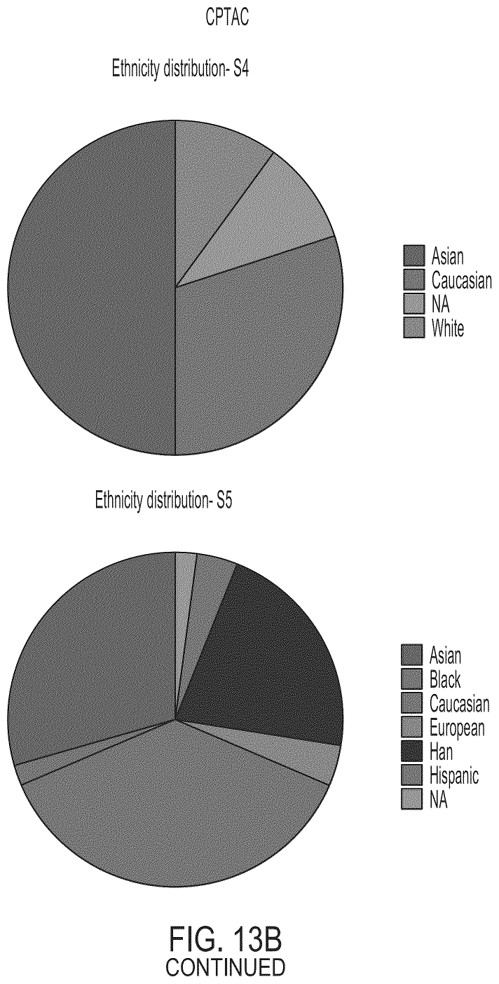

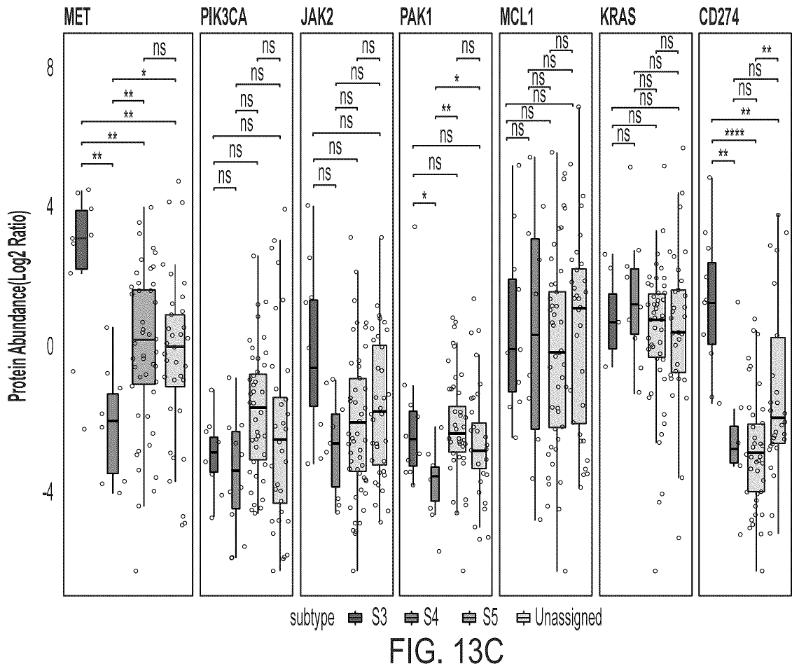

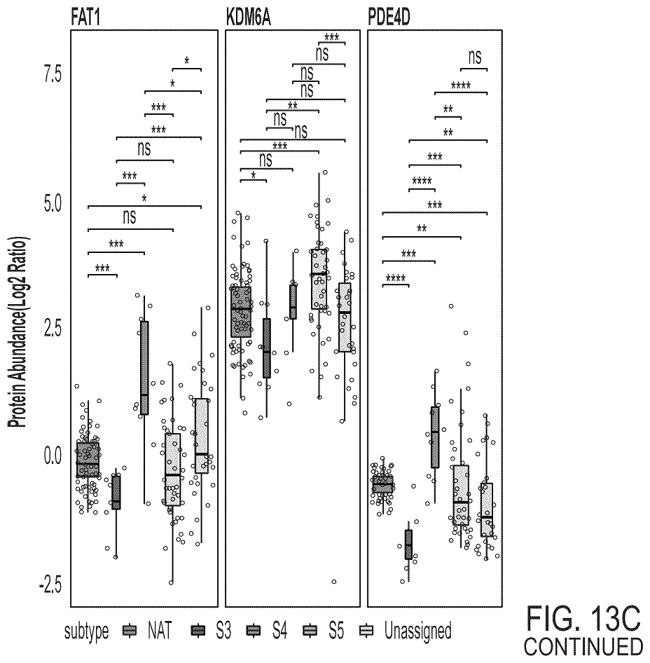

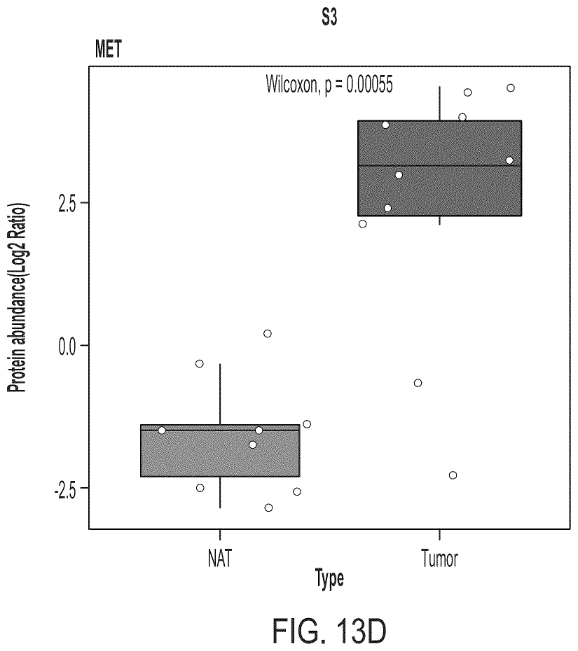

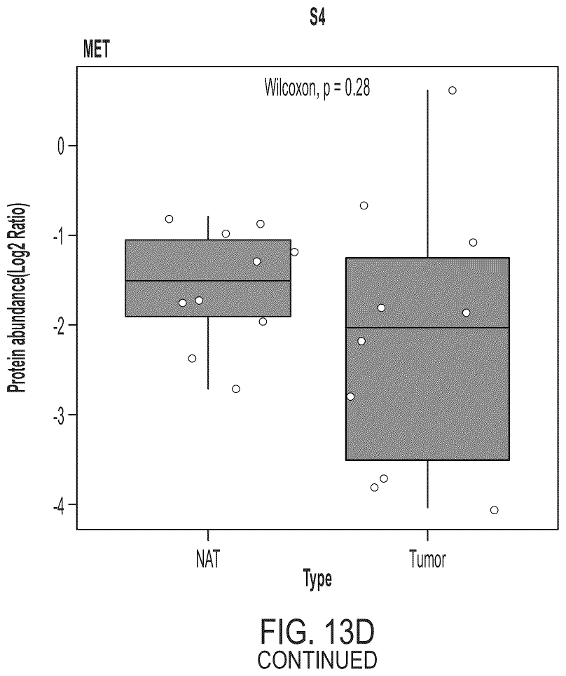

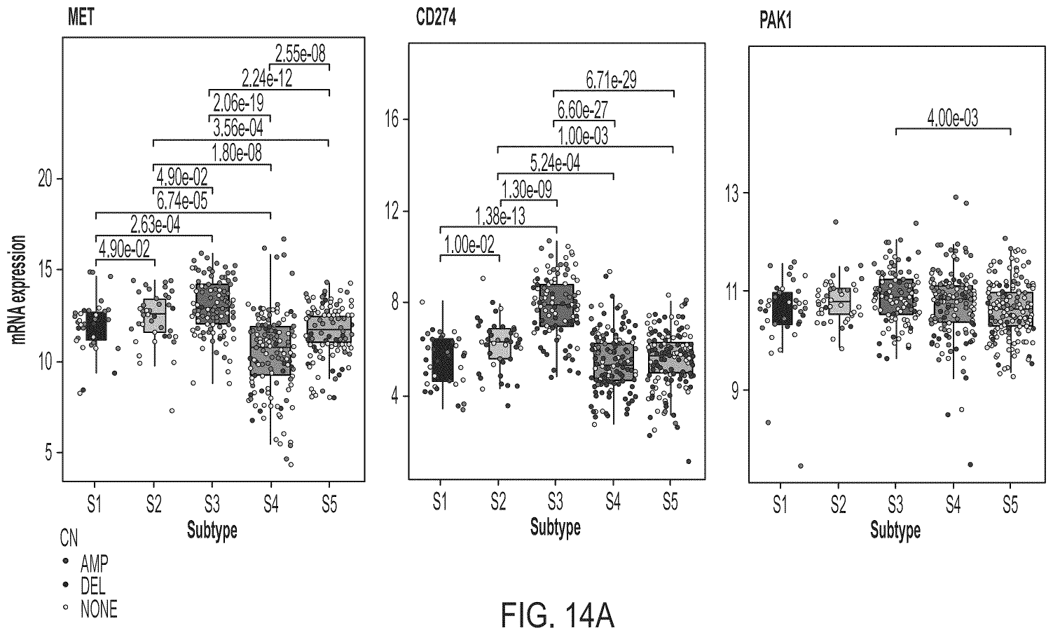

To further characterize the expression subtypes at the proteomics level, expression subtypes for the Clinical Proteomic Tumor Analysis Consortium (CPTAC) lung adenocarcinoma (LUAD) samples (Gillette, Michael A., et al. “Proteogenomic characterization reveals therapeutic vulnerabilities in lung adenocarcinoma.” Cell 182.1 (2020): 200-225) were annotated based on the expression of the subtype-specific marker genes (FIGS. 1A and 3A). Since S3 (n=13), S4 (n=13), and S5 (n=58) were the major subtypes represented in the CPTAC lung adenocarcinoma (LUAD) cohort, the downstream proteogenomic analysis focused on these subtypes. Consistent with the TCGA data analysis, both S3 and S4 showed increased proliferation signatures, and S3 also showed an increased immune/inflammatory signature (FIG. 3A). Comparing the expression subtypes with the CPTAC multi-omics clusters (Gillette, Michael A., et al. “Proteogenomic characterization reveals therapeutic vulnerabilities in lung adenocarcinoma.” Cell 182.1 (2020): 200-225), a good agreement was found: S3 was enriched with CPTAC multi-omics cluster C1 tumors (PI enriched; 13 C1 subtype tumors out of 13 S3 tumors), S4 was enriched with C3 tumors (PP enriched; 10 C3 subtype tumors out of 13 S4 tumors) (FIG. 13A), and S5 was enriched with C4 tumors (TRU enriched; 33 C4 subtype tumors out of 56 S5 tumors). Overall, the RNA expression subtypes significantly overlapped the previous mRNA subtypes in TCGA (orig, Cluster of Clusters Analysis (COCA)) and the multi-omic subtypes in CPTAC (Gillette, Michael A., et al. “Proteogenomic characterization reveals therapeutic vulnerabilities in lung adenocarcinoma.” Cell 182.1 (2020): 200-225), while identifying higher-resolution partitioning of the PI subtype (FIG. 1B).

Next, the genomic and proteomic features of each of the subtypes was further investigated. The overall frequency profiles of amplification or deletion of significantly copy-number altered genes was similar between the TCGA and CPTAC cohorts for S3, S4, and S5 (cosine similarities >0.93; FIG. 3B). Some differences could be attributed to the distinct populations represented in the two cohorts. While the TCGA lung adenocarcinoma (LUAD) cohort was mainly composed of Caucasian individuals, the Clinical Proteomic Tumor Analysis Consortium (CPTAC) lung adenocarcinoma (LUAD) cohort was more diverse, with approximately half Caucasian and half Asian individuals (FIG. 13B). Despite the difference in ethnic background, the fact that the profiles associated with each subtype were more similar to the corresponding subtype than to other subtypes lent further support to the subtype classification of the Clinical Proteomic Tumor Analysis Consortium (CPTAC) tumors.

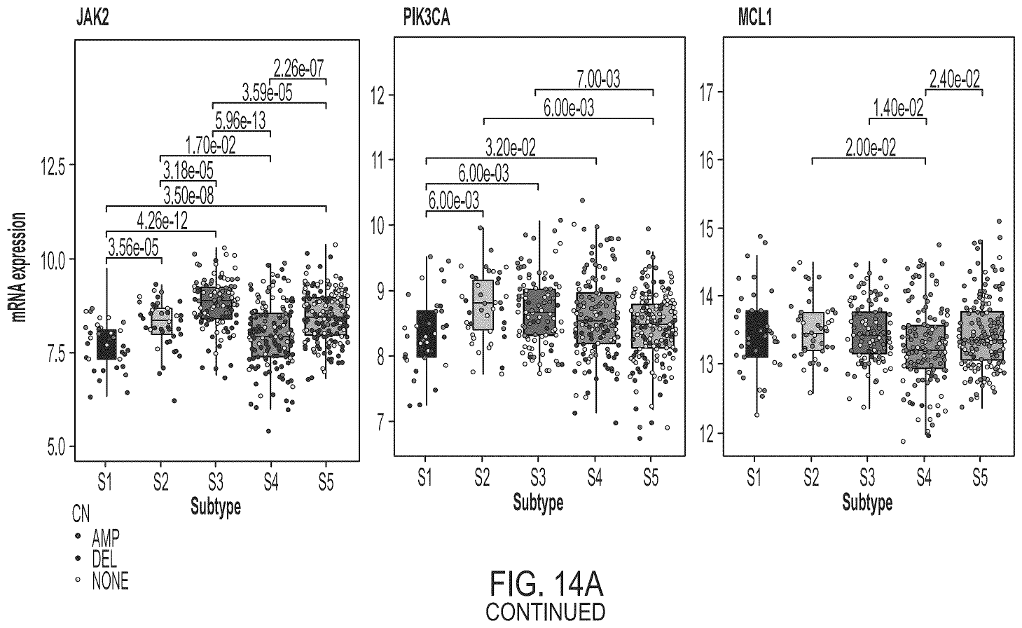

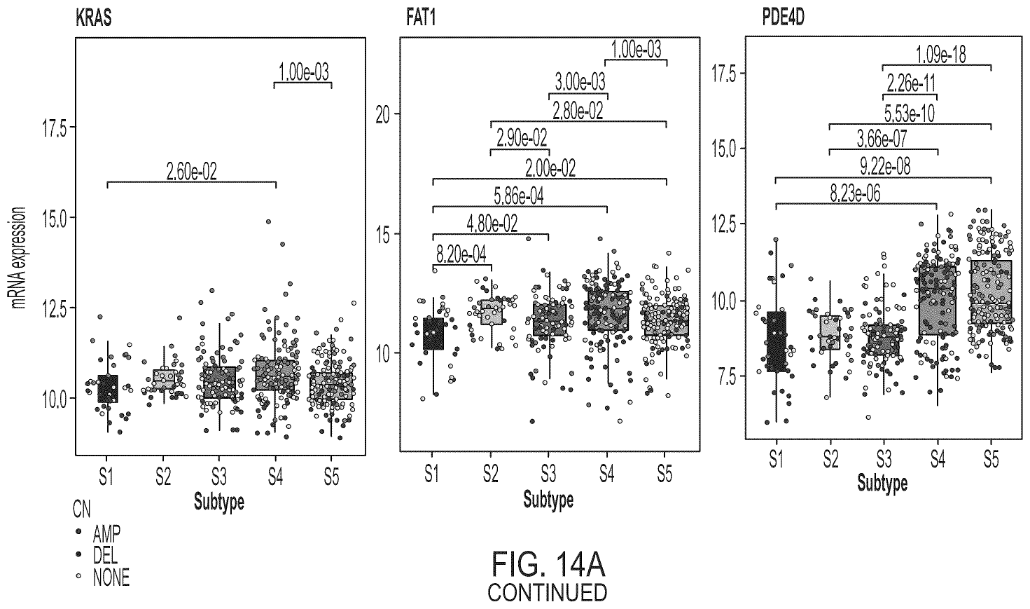

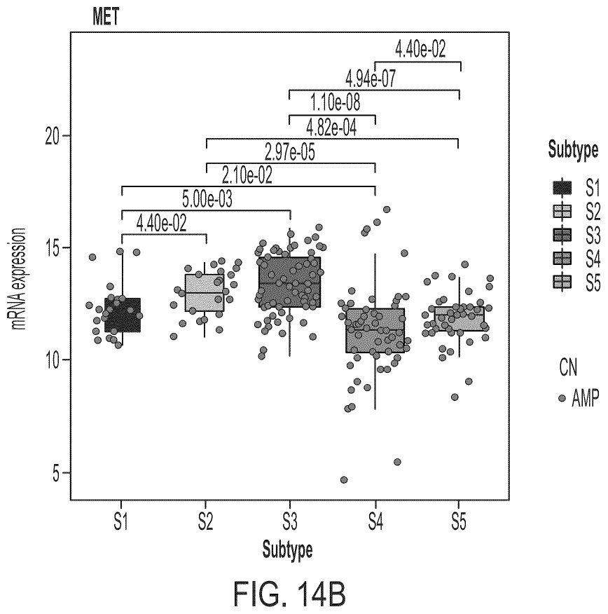

The effect of recurrent somatic copy-number alterations (SCNAs) on protein expression was then explored. Among the genes with recurrent SCNAs in the S3, S4, and S5 subtypes, the available protein expression for these genes was compared across S3, S4, S5, normal adjacent tissues (NAT), and Clinical Proteomic Tumor Analysis Consortium (CPTAC) lung adenocarcinoma (LUAD) samples with no assigned expression subtypes (designated as ‘unassigned’ due to lower similarity to any of the five expression subtypes) (FIGS. 3C and 13C). JAK2 and CD274 (PD-L1) showed both recurrent gene amplification and significantly higher protein expression in S3. Interestingly, MET showed recurrent gene amplification in both S3 and S4, but its protein expression was significantly up-regulated only in S3 (FIG. 3C), also exceeding the expression in normal adjacent tissues (P<5.5×10⁴) (FIG. 13D). Moreover, S3 tumor samples with MET amplification showed much higher MET protein expression than S3 tumors with no MET amplification, whereas other subtypes showed weaker (or no) correlation between MET amplification and MET protein expression. This finding suggested that S3 tumors may be responsive to MET inhibitors. Of all the genes that showed recurrent gene deletion in both S3 and S4, only FAT1 and PDE4D also exhibited significant changes to their proteomic expression. Moreover, only S3 exhibited a significantly downregulated protein expression for both FAT1 and PDE4D that was associated with their respective gene loss when compared to both NAT and the other subtypes. A similar trend was observed for mRNA expression in the TCGA lung adenocarcinoma (LUAD) cohort. These findings highlighted the need to take into account not only copy-number alterations but also mRNA and protein expression (FIG. 14A).

### Example 4: MET is a Core Regulator of Proliferation and PD-L1 Expression in S3

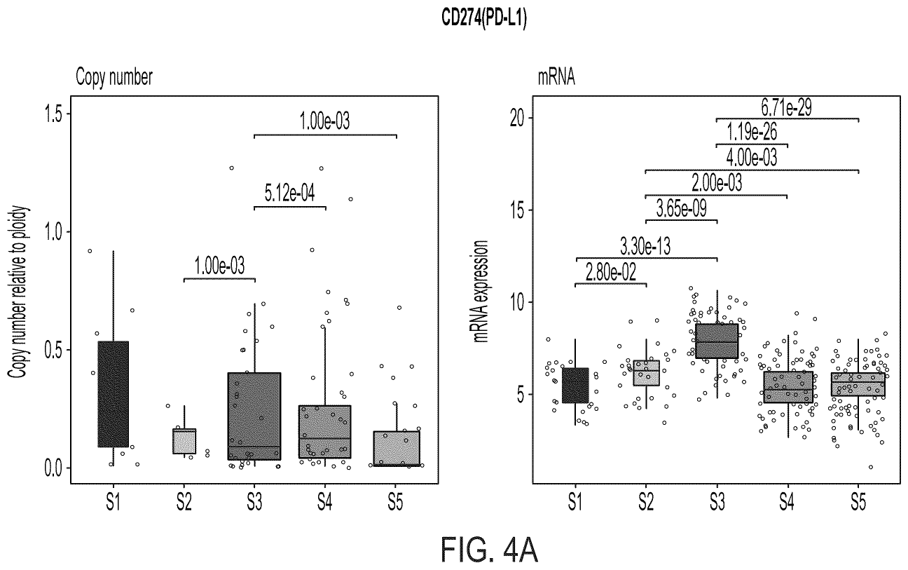

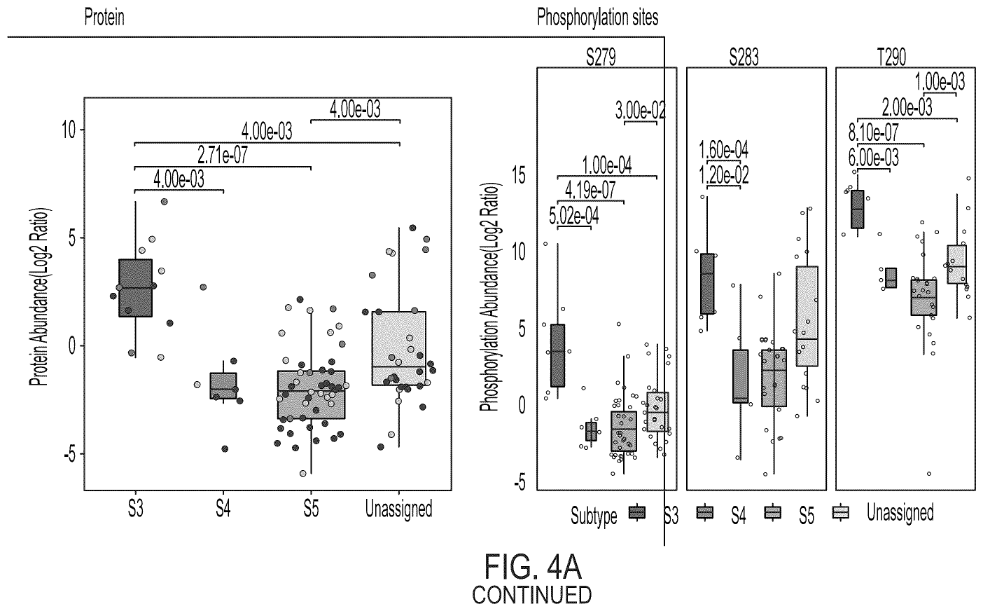

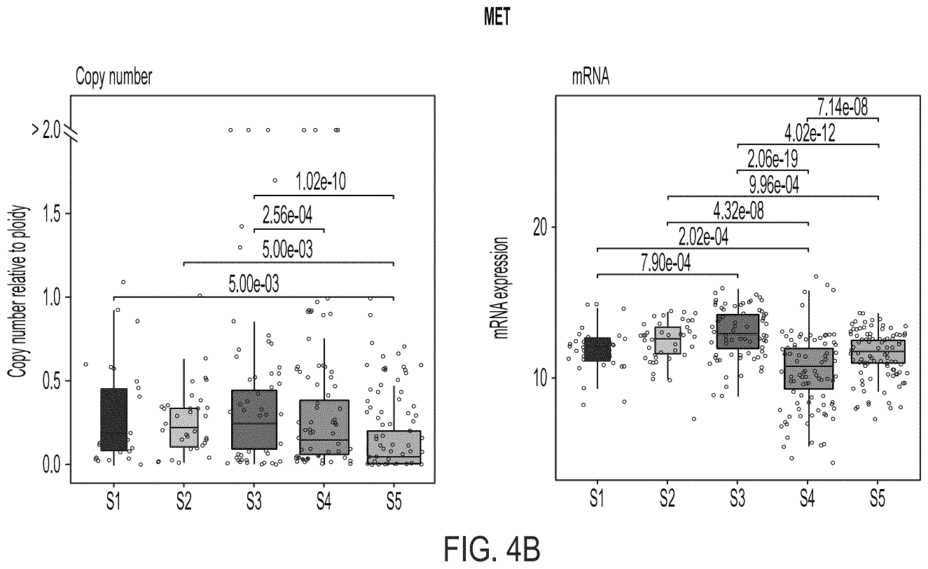

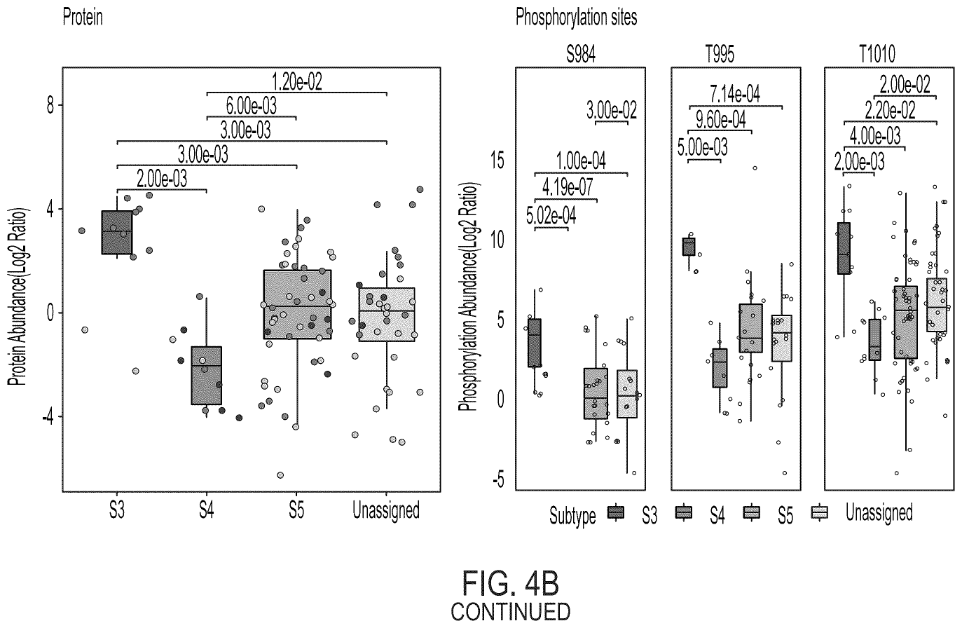

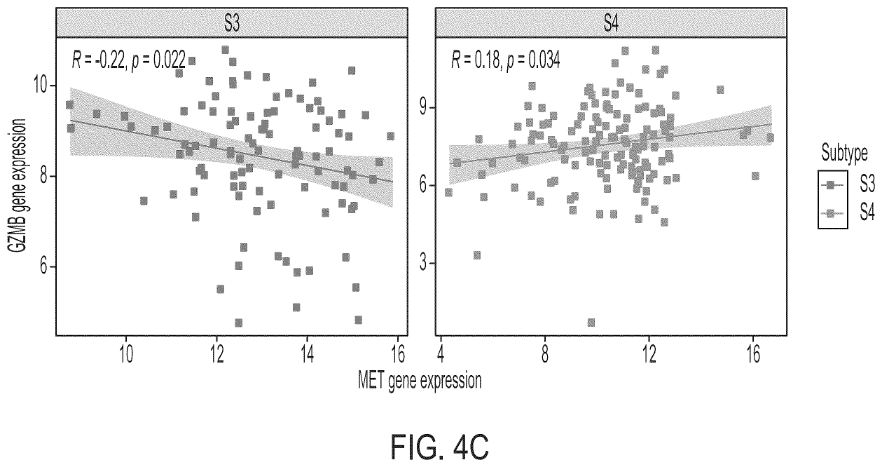

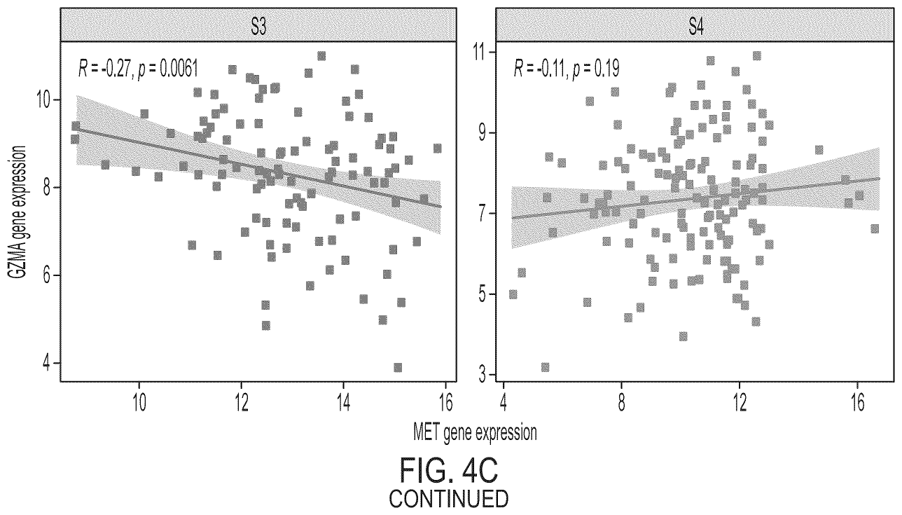

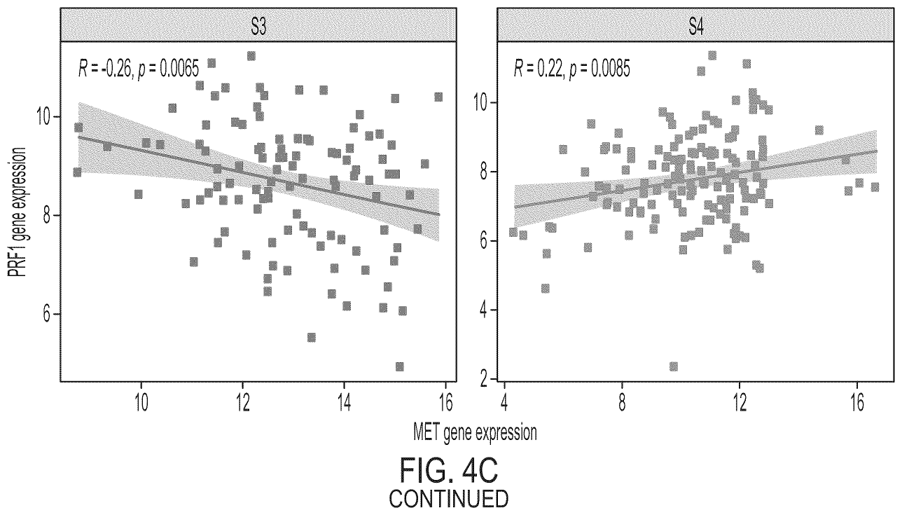

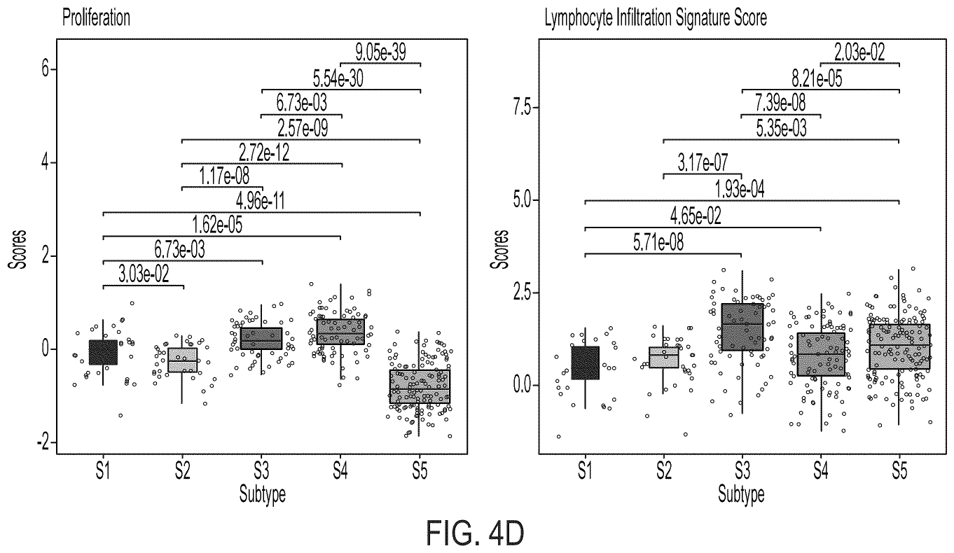

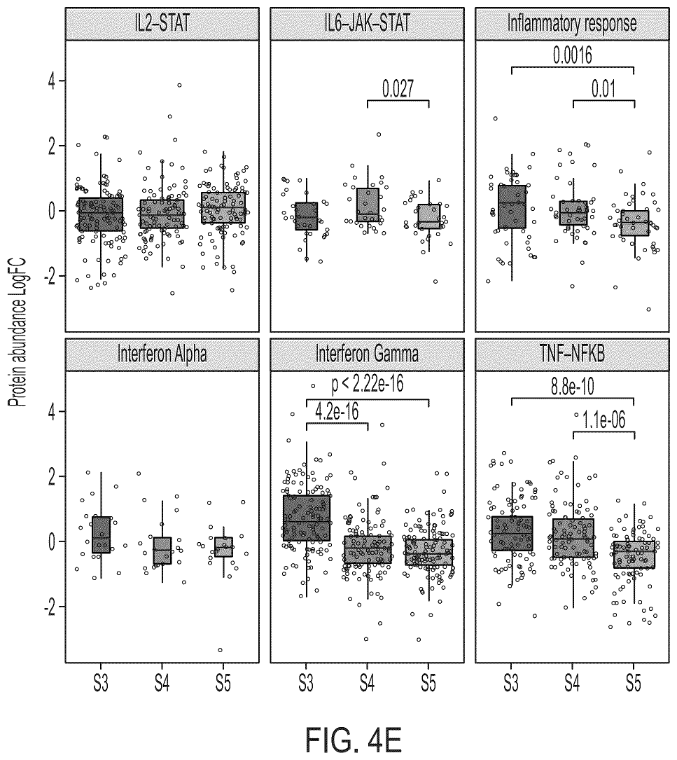

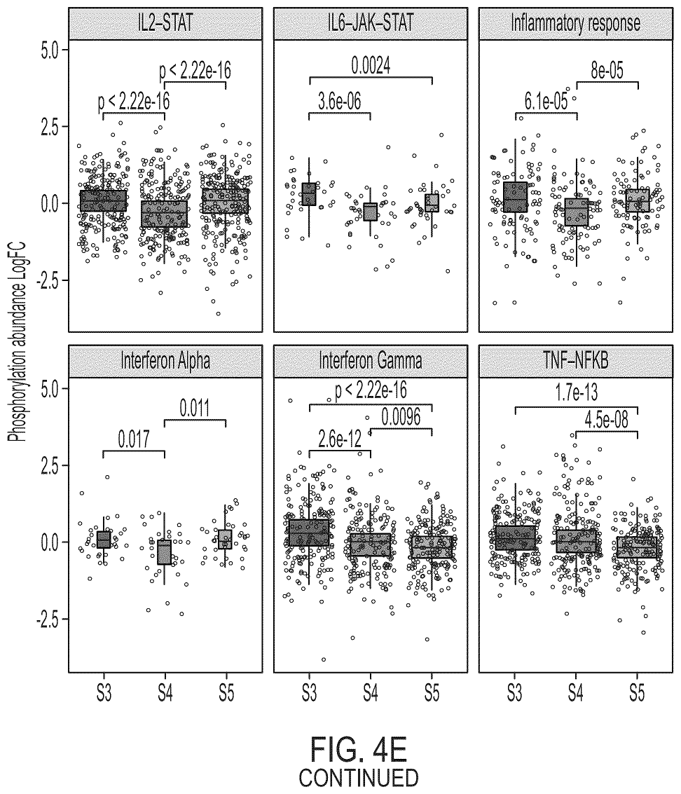

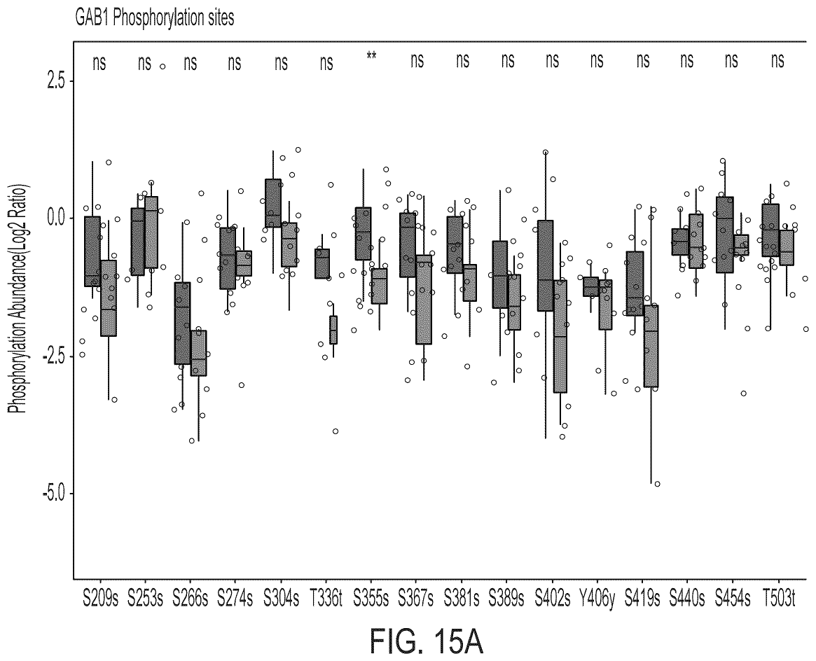

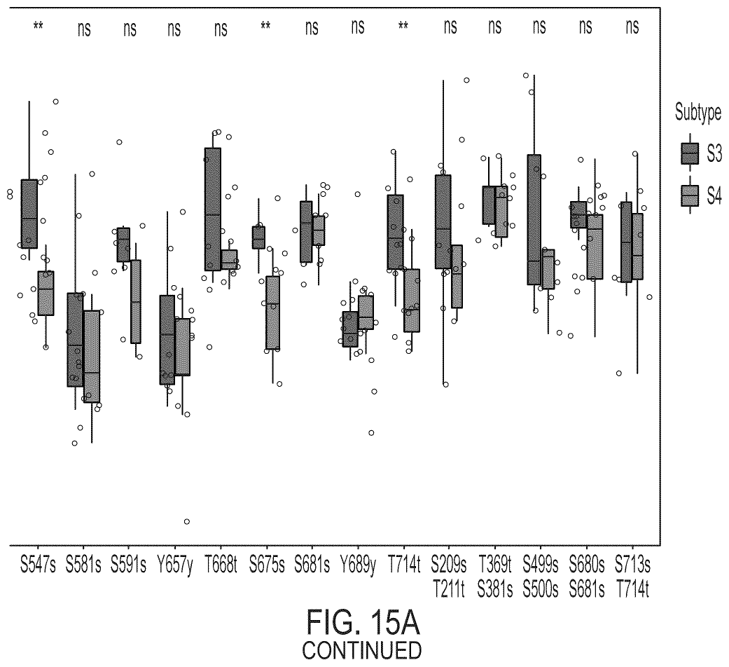

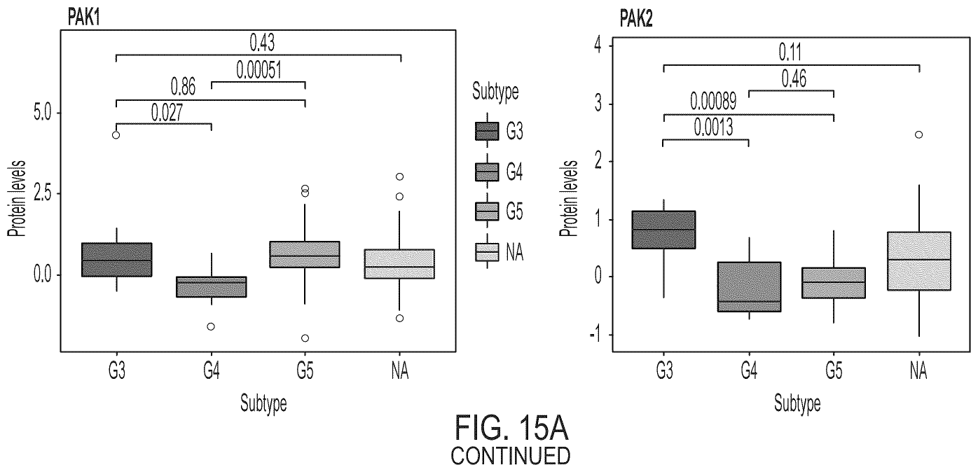

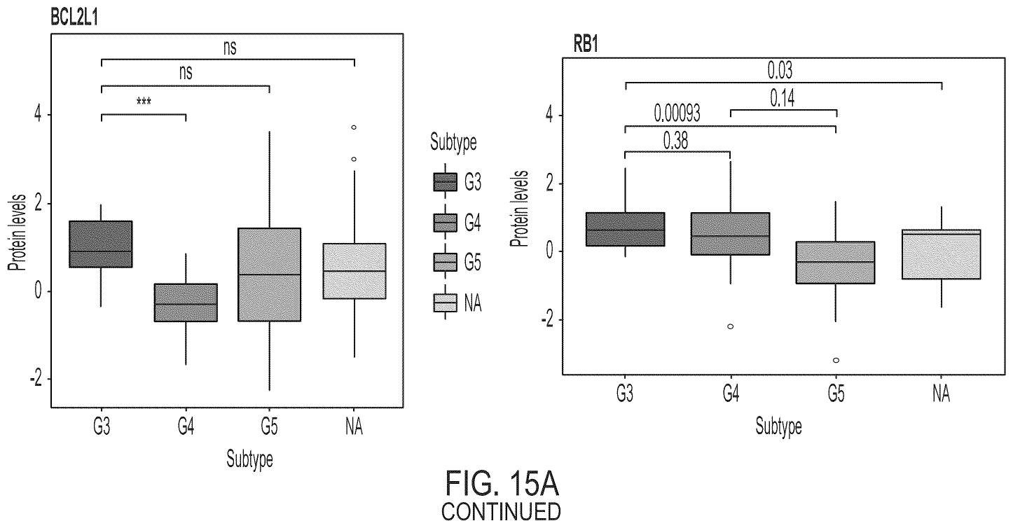

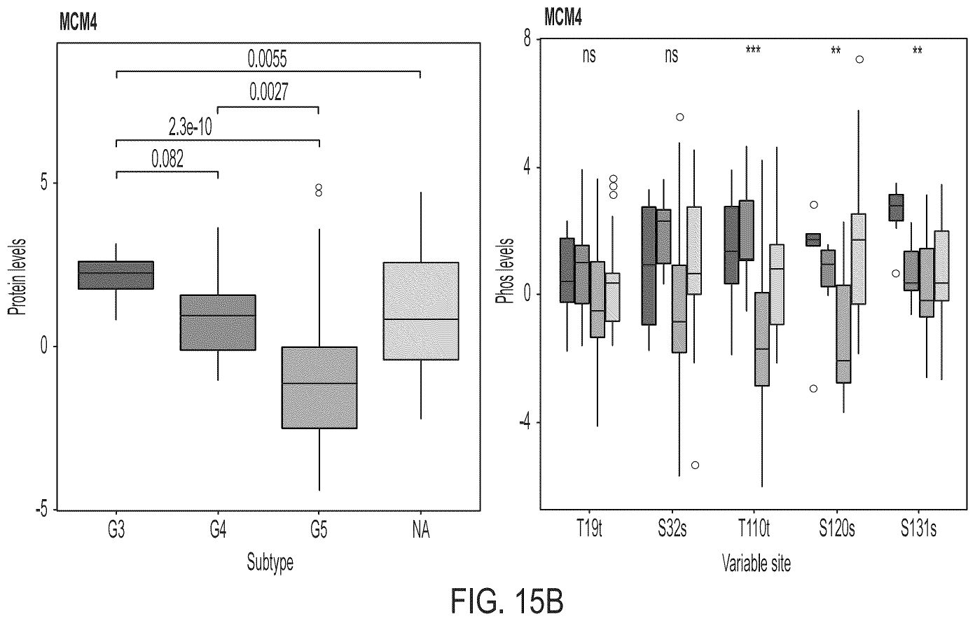

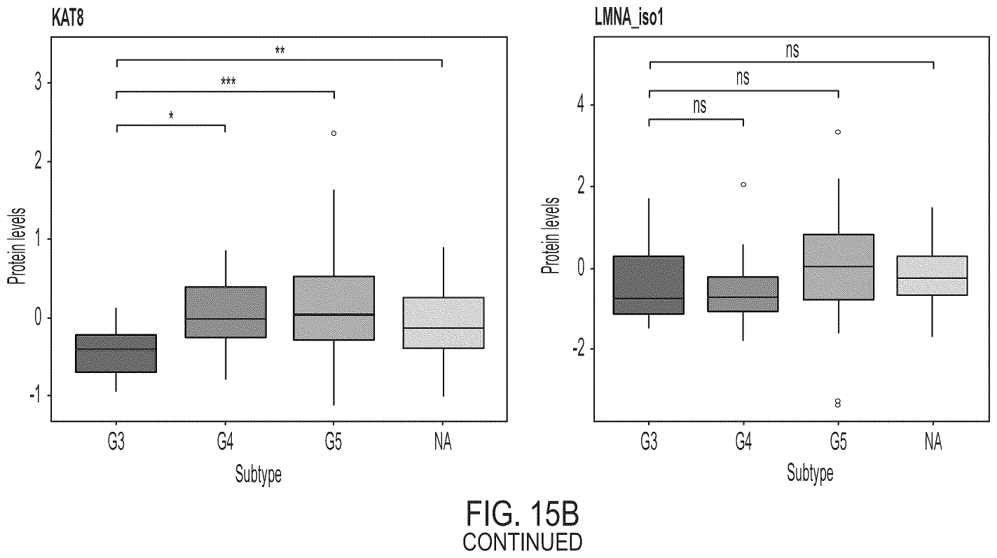

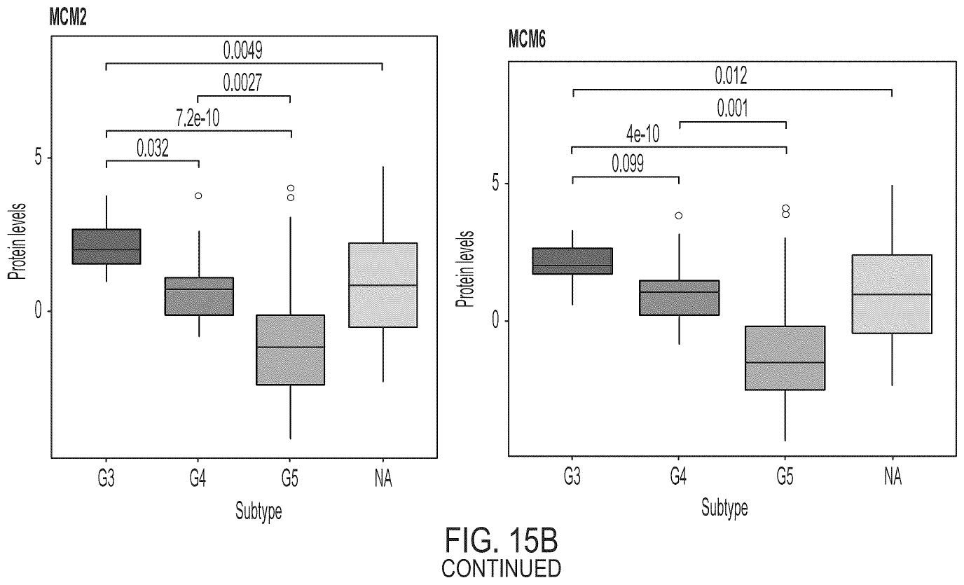

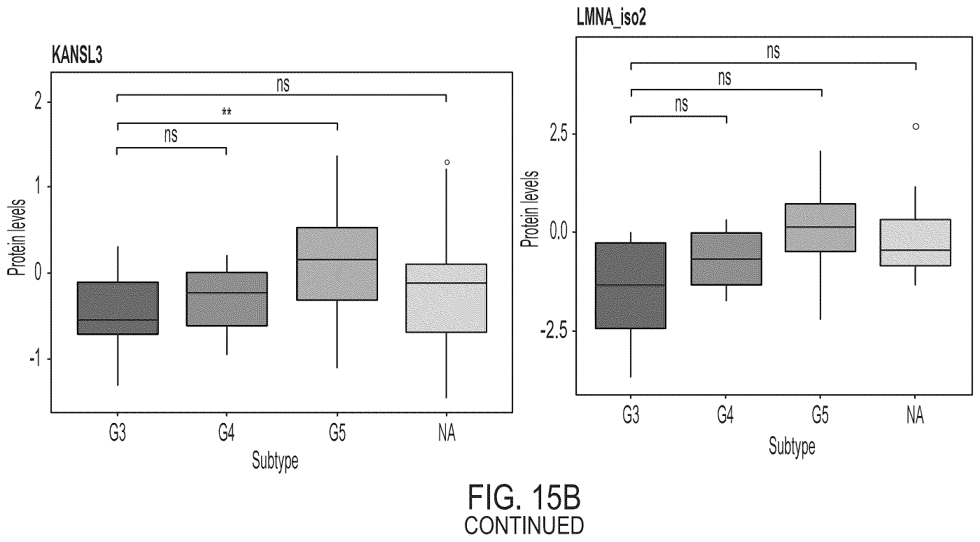

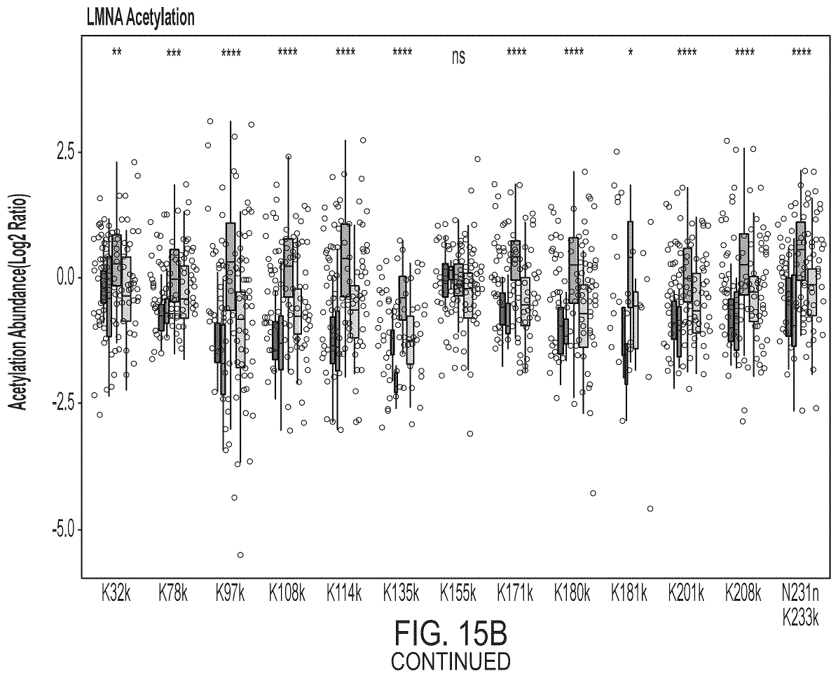

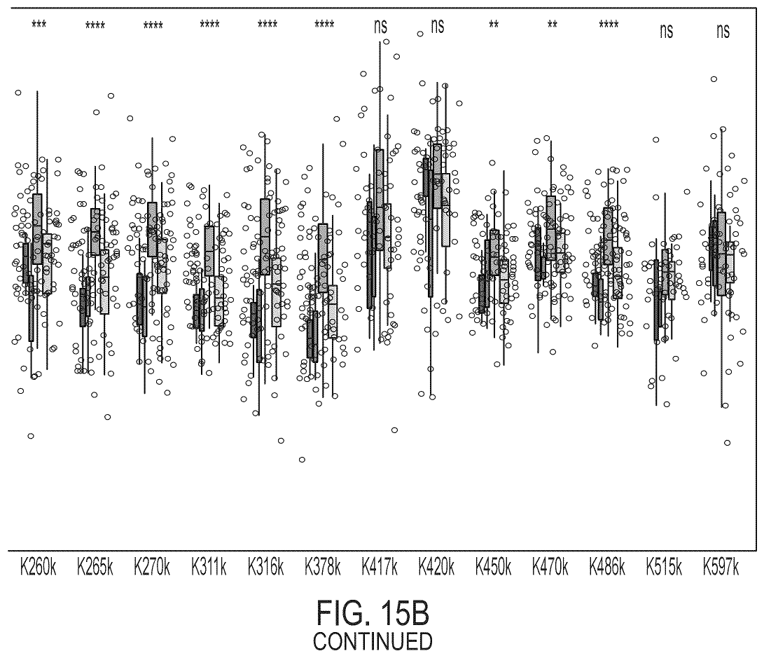

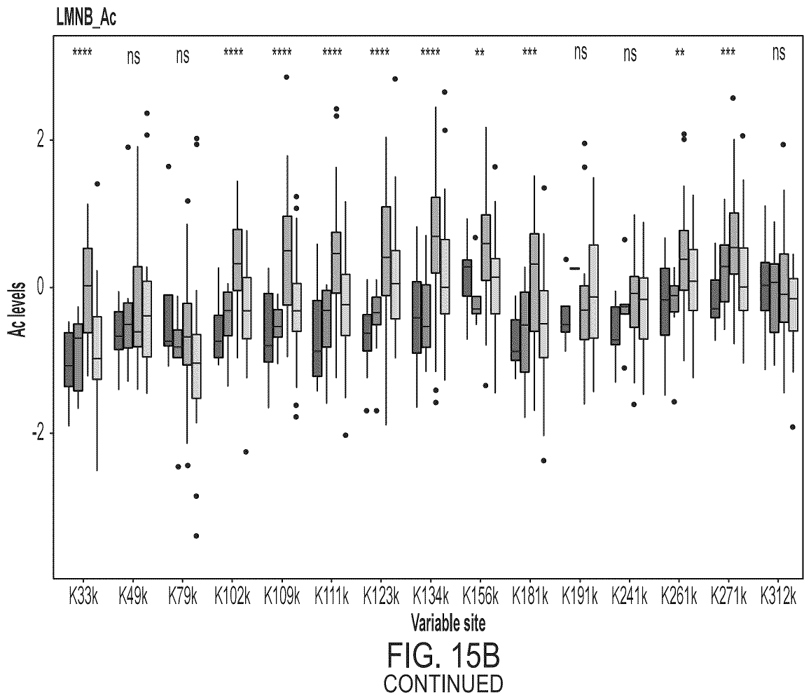

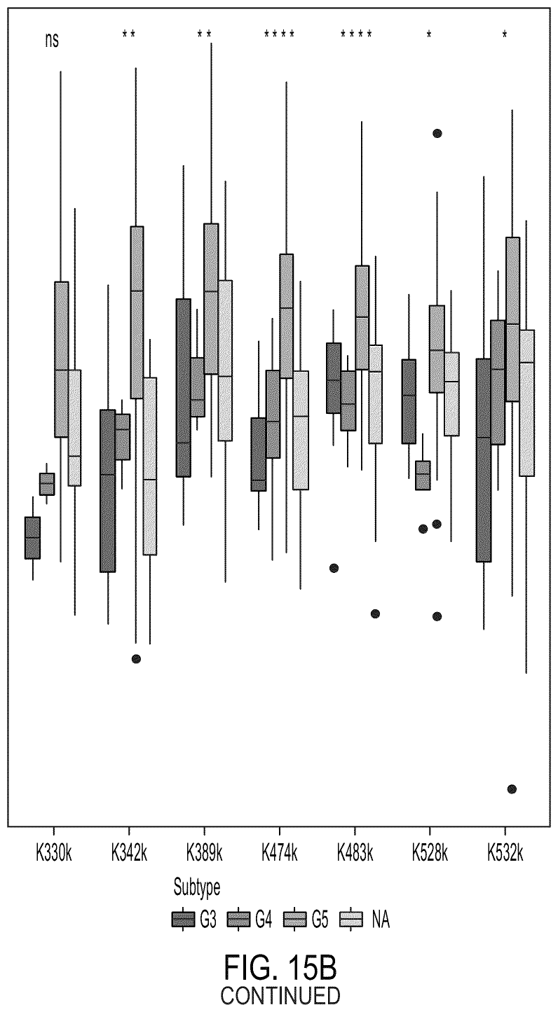

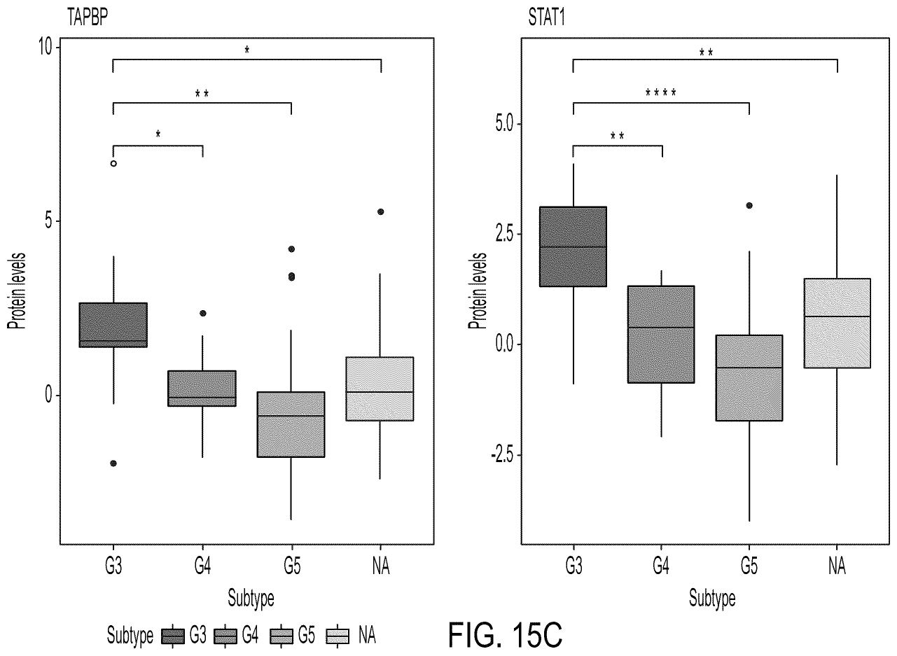

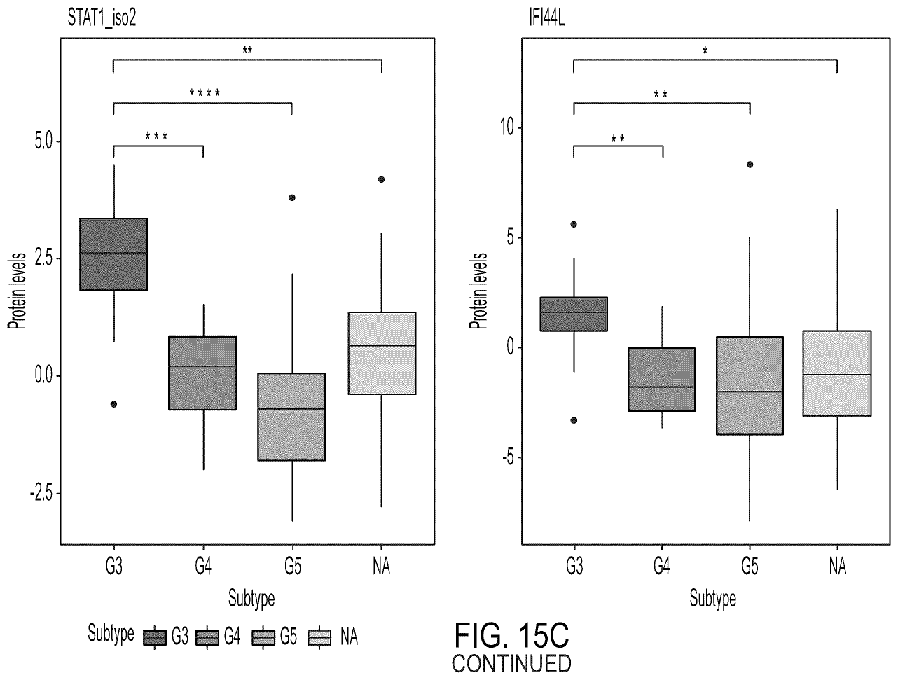

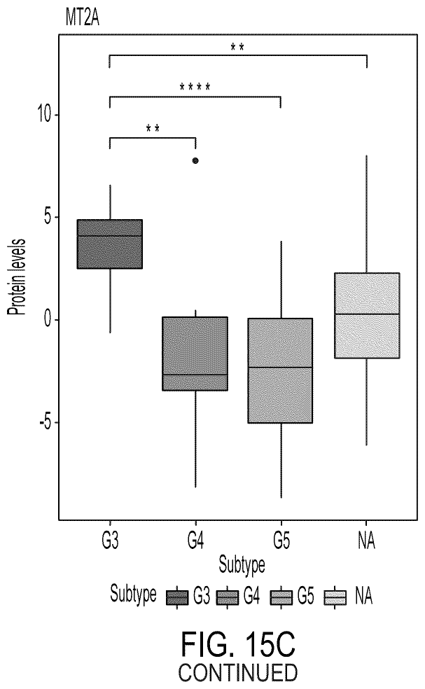

The results presented above suggested that S3 and S4 had a similarly high proliferative phenotype, and that only S3 showed a high immune phenotype. To gain additional insight into the underlying biological differences between S3 and S4 (as well as the differences among S1, S2, S5, and the unassigned samples that do not have these two phenotypes), a deeper proteogenomic characterization of the subtypes was performed. First, it was noted that CD274 (PD-L1) showed recurrent gene amplification in S3. It was further observed that PD-L1 copy number, mRNA expression, protein expression, and phosphorylation were significantly higher in S3 versus S4 (FIGS. 3B, 3C, and 4A). Since both S3 and S4 showed high proliferation signatures and recurrent MET amplification (FIGS. 1C and 9B), MET copy number and protein expression was assessed across subtypes and it was found that MET copy number was significantly higher in S3 versus S4, and that its expression of mRNA, protein, and phosphorylation was also higher in S3 versus S4 (FIGS. 3B-3C and 4B), echoing the expression pattern of PD-L1. The mRNA and protein expression of MET were also significantly higher in S3 versus S4, even when restricting the analysis only to MET-amplified tumors (Q=1.1×10⁻⁸ for mRNA, Q=2.7×10⁻²) (FIG. 14B). Additionally, higher MET pathway activation was identified in S3 versus S4 as evidenced by increased phosphorylation levels of GAB1 in S3, a known downstream substrate of MET (FIG. 15A). It was then investigated whether higher MET expression in S3 was associated with lower expression of T cell effector molecules. Interestingly, a negative correlation was observed between the expression of MET and T cell effector molecules in S3, but not in S4 (FIG. 4C). Not intending to be bound by theory, this may suggest immune evasion of S3 tumors associated with MET overexpression.

Proliferation and immune signatures were then evaluated using previously developed scores for proliferation and lymphocyte-infiltration (Thorsson, Vésteinn, et al. “The immune landscape of cancer.” Immunity 48.4 (2018): 812-830). While high proliferation scores were found in both S3 and S4, only S3 exhibited a high immune score (FIGS. 1C and 4D). Additionally, S3 had a significantly higher fraction of anti-tumoral M1 macrophages. Not wishing to be bound by theory, this may suggest a favorable tumor immune microenvironment for therapy, whereas S4 showed a significantly higher fraction of pro-tumoral Th2 cells (FIGS. 11A and 11B). Increased interferon-gamma pathway activity in S3 was also observed compared to S4 and S5 based on protein expression and phosphorylation data (FIG. 4E). This finding was further supported by the increased expression of proteins involved in antigen presentation and interferon signaling in S3 (FIG. 15C). Taken together, these proteomic findings supported increased immune/inflammatory activity in S3.

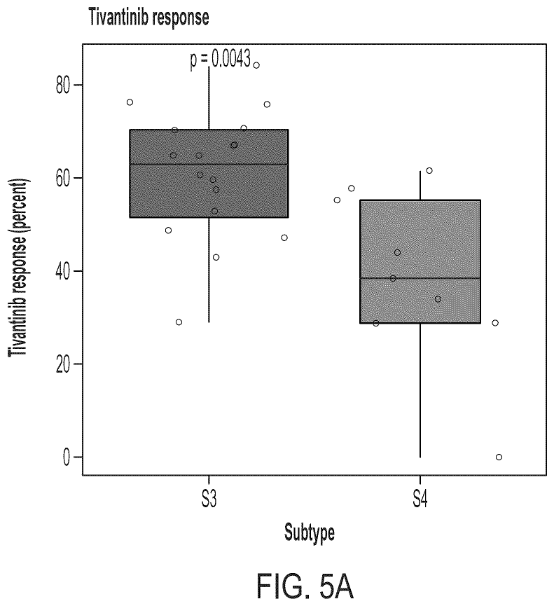

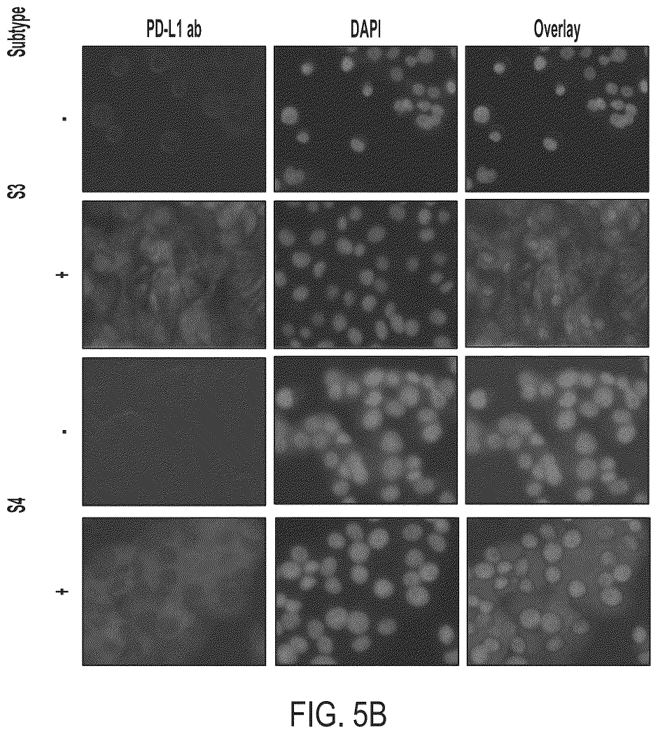

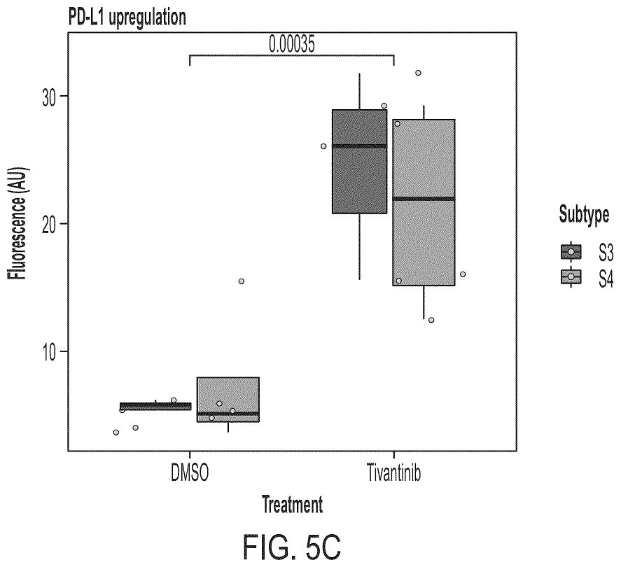

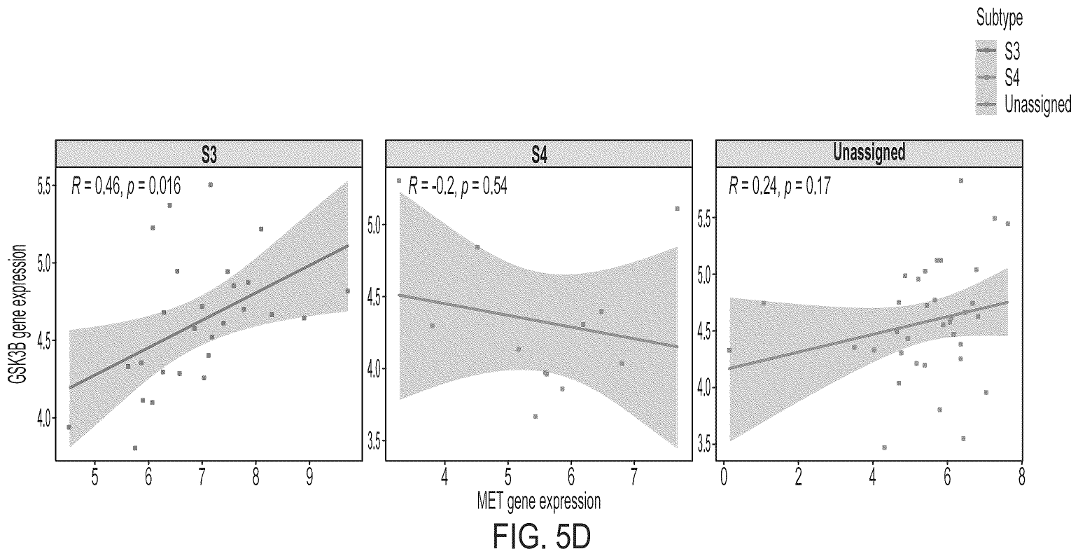

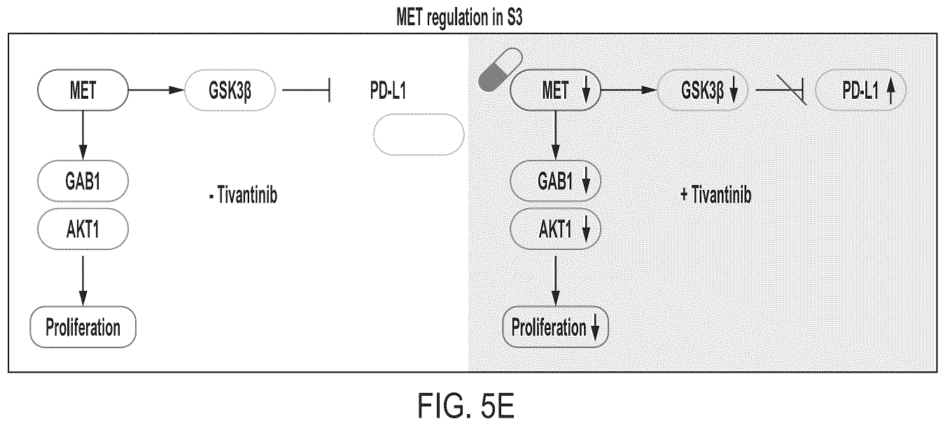

To further support and validate the above findings, the response of subtype-specific cell lines (described above) to the MET inhibitor, Tivantinib, was investigated. Tivantinib is a non-ATP-competitive c-Met inhibitor that induces G2/M arrest and apoptosis. A 4-day long proliferation assay was performed to test the response in the different cell lines. S3 showed a significantly increased response (P value >0.001) to tivantinib treatment compared to the other assigned groups (FIG. 5A). To test whether PC-L1 expression was enhanced in response to c-MET inhibition, immunofluorescence staining was performed on the tivantinib-treated cells and controls with an anti-PD-L1 antibody to monitor PD-L1 levels. A significant increase in PD-L1 levels was detected in all subtypes in response to tivantinib (Wilcoxon test P value >0.0001) (FIGS. 5B and 5C). The correlation of mRNA expression between MET and GSK3β in the different lung adenocarcinoma (LUAD) cell line data was next texted and a significant positive correlation was found only in the S3 subtype (Pearson correlation coefficient=0.46, P value=0.016) (FIG. 5D).

Collectively, while not intending to be bound by theory, the above data suggested that MET is a core regulator of increased proliferation of cancer cells through GAB1/AKT1, and MET can also upregulate PD-L1 expression through the GSK3β axis, potentially for immune escape. Additional synergistic players, found to have higher protein expression in S3 vs S4, such as BCL2L1 and the MCM family, also likely further contribute to the proliferation of cells in S3 (FIGS. 5E and 15B).

### Example 5: Biomarkers for Identifying Patients with S2, S3, or S4 Tumors

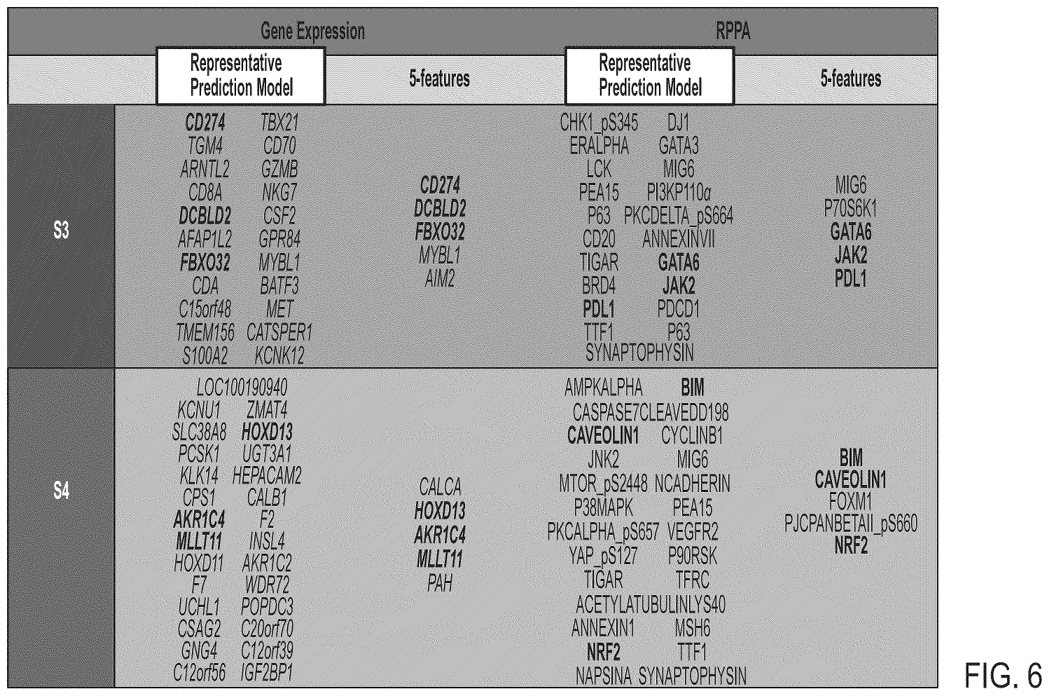

Biomarkers for S2, S3, and S4 were identified so that patients with these subtypes could be readily identified in the clinic. For gene-expression data, the subtype marker genes defined above (Table 15) were used as the potential features to test. A representative prediction model for S3 (23 genes) showed a model accuracy of 95%, and a representative prediction model for S4 (27 genes) showed a model accuracy of 85% (FIG. 6 and Tables 7-14). TCGA reverse-phase protein array (RPPA) data was also considered as potential proteomic features. Representative prediction models for S3 and S4 contained 20 and 24 protein features, respectively, and were each 91% accurate. Additionally, for better clinical utility, the model was forced to reduce the number of features down to five. Interestingly, the five-feature model for S3 based on RPPA data (PD-L1, JAK2, MIG6, P70S6K1, GATA6) still showed a high model accuracy of 91%, and the five-feature model for S4 based on RPPA data (BIM, CAVEOLIN1, FOXM1, PKCPANBETAII_pS660, NRF2) showed a model accuracy of 74%. These results showed that high prediction accuracies for S3 and S4 can be achieved. This was demonstrated using both gene expression and RPPA data. Representative models for S2 are provided in Tables 16-19.

Tables 7-14 and 16-19 present results from a biomarker discovery analysis carried out by lasso logistic regression models.

TABLE 7A
Results from S3 prediction models based on The Cancer Genome
Atlas (TCGA) lung adenocarcinoma (LUAD) gene expression data.

| Lambda | Predictors | Coefficients | Threshold | Accuracy |
|---|---|---|---|---|
| 0.0163 | (Intercept) | −25.080 | 0.5 | 0.950 |
| | CD274 | 0.272 | | |
| | TBX21 | 0.241 | | |
| | TGM4 | 0.118 | | |
| | CD70 | 0.041 | | |
| | ARNTL2 | 0.107 | | |
| | GZMB | 0.149 | | |
| | CD8A | 0.189 | | |
| | NKG7 | 0.065 | | |
| | DCBLD2 | 0.230 | | |
| | CSF2 | 0.020 | | |
| | AFAP1L2 | 0.199 | | |
| | GPR84 | 0.113 | | |
| | FBXO32 | 0.360 | | |
| | MYBL1 | 0.528 | | |
| | CDA | 0.026 | | |
| | BATF3 | 0.228 | | |
| | C15orf48 | 0.069 | | |
| | MET | 0.068 | | |
| | TMEM156 | 0.019 | | |
| | CATSPER1 | 0.136 | | |
| | S100A2 | 0.061 | | |
| | KCNK12 | 0.011 | | |

TABLE 7B
Results from S3 prediction models based on The
Cancer Genome Atlas (TCGA)
lung adenocarcinoma (LUAD) gene expression data.

| Lambda | Predictors | Coefficients | Threshold | Accuracy |
|---|---|---|---|---|
| 0.126 | (Intercept) | −5.620 | 0.2 | 0.832 |
| | CD274 | 0.391 | | |
| | AIM2 | 0.003 | | |
| | DCBLD2 | 0.103 | | |
| | FBXO32 | 0.062 | | |
| | MYBL1 | 0.017 | | |

TABLE 8
Accuracy of S3 prediction models based on The Cancer
Genome Atlas (TCGA) lung adenocarcinoma (LUAD)
gene expression data with varying lambda values. The five-feature
model is in bold. Each model is preceded by the Intercept
corresponding to the model.

| Lambda | Predictors | Coefficients | Accuracy |
|---|---|---|---|
| 0.0163 | (Intercept) | −25.080 | 0.950 |
| 0.0163 | CD274 | 0.272 | 0.950 |
| 0.0163 | TBX21 | 0.241 | 0.950 |
| 0.0163 | TGM4 | 0.118 | 0.950 |
| 0.0163 | CD70 | 0.041 | 0.950 |
| 0.0163 | ARNTL2 | 0.107 | 0.950 |
| 0.0163 | GZMB | 0.149 | 0.950 |
| 0.0163 | CD8A | 0.189 | 0.950 |
| 0.0163 | NKG7 | 0.065 | 0.950 |
| 0.0163 | DCBLD2 | 0.230 | 0.950 |
| 0.0163 | CSF2 | 0.020 | 0.950 |
| 0.0163 | AFAPIL2 | 0.199 | 0.950 |
| 0.0163 | GPR84 | 0.113 | 0.950 |
| 0.0163 | FBXO32 | 0.360 | 0.950 |
| 0.0163 | MYBL1 | 0.528 | 0.950 |
| 0.0163 | CDA | 0.026 | 0.950 |
| 0.0163 | BATF3 | 0.228 | 0.950 |
| 0.0163 | C15orf48 | 0.069 | 0.950 |
| 0.0163 | MET | 0.068 | 0.950 |
| 0.0163 | TMEM156 | 0.019 | 0.950 |
| 0.0163 | CATSPER1 | 0.136 | 0.950 |
| 0.0163 | S100A2 | 0.061 | 0.950 |
| 0.0163 | KCNK12 | 0.011 | 0.950 |
| 0.0263 | (Intercept) | −19.754 | 0.941 |
| 0.0263 | CD274 | 0.337 | 0.941 |
| 0.0263 | TBX21 | 0.181 | 0.941 |
| 0.0263 | TGM4 | 0.109 | 0.941 |
| 0.0263 | CD70 | 0.027 | 0.941 |
| 0.0263 | ARNTL2 | 0.043 | 0.941 |
| 0.0263 | GZMB | 0.123 | 0.941 |
| 0.0263 | CD8A | 0.093 | 0.941 |
| 0.0263 | IFNG | 0.014 | 0.941 |
| 0.0263 | NKG7 | 0.085 | 0.941 |
| 0.0263 | DCBLD2 | 0.220 | 0.941 |
| 0.0263 | AFAPIL2 | 0.160 | 0.941 |
| 0.0263 | GPR84 | 0.050 | 0.941 |
| 0.0263 | FBXO32 | 0.289 | 0.941 |
| 0.0263 | MYBL1 | 0.388 | 0.941 |
| 0.0263 | CDA | 0.038 | 0.941 |
| 0.0263 | BATF3 | 0.171 | 0.941 |
| 0.0263 | C15orf48 | 0.044 | 0.941 |
| 0.0263 | MET | 0.021 | 0.941 |
| 0.0263 | CATSPER1 | 0.135 | 0.941 |
| 0.0263 | S100A2 | 0.031 | 0.941 |
| 0.0363 | (Intercept) | −16.312 | 0.931 |
| 0.0363 | CD274 | 0.374 | 0.931 |
| 0.0363 | TBX21 | 0.137 | 0.931 |
| 0.0363 | TGM4 | 0.091 | 0.931 |
| 0.0363 | CD70 | 0.014 | 0.931 |
| 0.0363 | GZMB | 0.117 | 0.931 |
| 0.0363 | CD8A | 0.023 | 0.931 |
| 0.0363 | IFNG | 0.032 | 0.931 |
| 0.0363 | NKG7 | 0.082 | 0.931 |
| 0.0363 | DCBLD2 | 0.215 | 0.931 |
| 0.0363 | C12orf70 | 0.012 | 0.931 |
| 0.0363 | AFAPIL2 | 0.136 | 0.931 |
| 0.0363 | FBXO32 | 0.251 | 0.931 |
| 0.0363 | MYBL1 | 0.296 | 0.931 |
| 0.0363 | CDA | 0.043 | 0.931 |
| 0.0363 | C10orf55 | 0.006 | 0.931 |
| 0.0363 | BATF3 | 0.132 | 0.931 |
| 0.0363 | C15orf48 | 0.024 | 0.931 |
| 0.0363 | CATSPER1 | 0.122 | 0.931 |
| 0.0363 | S100A2 | 0.003 | 0.931 |
| 0.0463 | (Intercept) | −14.141 | 0.921 |
| 0.0463 | CD274 | 0.380 | 0.921 |
| 0.0463 | TBX21 | 0.110 | 0.921 |
| 0.0463 | TGM4 | 0.068 | 0.921 |
| 0.0463 | CD70 | 0.005 | 0.921 |
| 0.0463 | GZMB | 0.113 | 0.921 |
| 0.0463 | IFNG | 0.031 | 0.921 |
| 0.0463 | NKG7 | 0.060 | 0.921 |
| 0.0463 | DCBLD2 | 0.200 | 0.921 |
| 0.0463 | AFAP1L2 | 0.107 | 0.921 |
| 0.0463 | FBXO32 | 0.220 | 0.921 |
| 0.0463 | MYBL1 | 0.241 | 0.921 |
| 0.0463 | CDA | 0.043 | 0.921 |
| 0.0463 | C10orf55 | 0.006 | 0.921 |
| 0.0463 | BATF3 | 0.105 | 0.921 |
| 0.0463 | C15orf48 | 0.002 | 0.921 |
| 0.0463 | CATSPER1 | 0.107 | 0.921 |
| 0.0563 | (Intercept) | −12.456 | 0.911 |
| 0.0563 | CD274 | 0.385 | 0.911 |
| 0.0563 | TBX21 | 0.094 | 0.911 |
| 0.0563 | TGM4 | 0.048 | 0.911 |
| 0.0563 | GZMB | 0.107 | 0.911 |
| 0.0563 | IFNG | 0.026 | 0.911 |
| 0.0563 | NKG7 | 0.024 | 0.911 |
| 0.0563 | DCBLD2 | 0.185 | 0.911 |
| 0.0563 | AFAP1L2 | 0.081 | 0.911 |
| 0.0563 | FBXO32 | 0.191 | 0.911 |
| 0.0563 | MYBL1 | 0.199 | 0.911 |
| 0.0563 | CDA | 0.039 | 0.911 |
| 0.0563 | C10orf55 | 0.002 | 0.911 |
| 0.0563 | BATF3 | 0.080 | 0.911 |
| 0.0563 | CATSPER1 | 0.092 | 0.911 |
| 0.0663 | (Intercept) | −11.102 | 0.891 |
| 0.0663 | CD274 | 0.389 | 0.891 |
| 0.0663 | GBP1 | 0.005 | 0.891 |
| 0.0663 | TBX21 | 0.077 | 0.891 |
| 0.0663 | TGM4 | 0.030 | 0.891 |
| 0.0663 | GZMB | 0.099 | 0.891 |
| 0.0663 | IFNG | 0.018 | 0.891 |
| 0.0663 | DCBLD2 | 0.172 | 0.891 |
| 0.0663 | AFAPIL2 | 0.060 | 0.891 |
| 0.0663 | FBXO32 | 0.167 | 0.891 |
| 0.0663 | MYBL1 | 0.164 | 0.891 |
| 0.0663 | CDA | 0.034 | 0.891 |
| 0.0663 | BATF3 | 0.057 | 0.891 |
| 0.0663 | CATSPER1 | 0.077 | 0.891 |
| 0.0763 | (Intercept) | −10.069 | 0.871 |
| 0.0763 | CD274 | 0.391 | 0.871 |
| 0.0763 | GBP1 | 0.022 | 0.871 |
| 0.0763 | TBX21 | 0.055 | 0.871 |
| 0.0763 | AIM2 | 0.002 | 0.871 |
| 0.0763 | TGM4 | 0.014 | 0.871 |
| 0.0763 | GZMB | 0.080 | 0.871 |
| 0.0763 | IFNG | 0.002 | 0.871 |
| 0.0763 | DCBLD2 | 0.160 | 0.871 |
| 0.0763 | AFAP1L2 | 0.041 | 0.871 |
| 0.0763 | FBXO32 | 0.144 | 0.871 |
| 0.0763 | MYBL1 | 0.139 | 0.871 |
| 0.0763 | CDA | 0.029 | 0.871 |
| 0.0763 | BATF3 | 0.037 | 0.871 |
| 0.0763 | CATSPER1 | 0.061 | 0.871 |
| 0.0863 | (Intercept) | −9.050 | 0.871 |
| 0.0863 | CD274 | 0.396 | 0.871 |
| 0.0863 | GBP1 | 0.027 | 0.871 |
| 0.0863 | TBX21 | 0.028 | 0.871 |
| 0.0863 | AIM2 | 0.006 | 0.871 |
| 0.0863 | TGM4 | 0.001 | 0.871 |
| 0.0863 | GZMB | 0.060 | 0.871 |
| 0.0863 | DCBLD2 | 0.149 | 0.871 |
| 0.0863 | AFAP1L2 | 0.027 | 0.871 |
| 0.0863 | FBXO32 | 0.126 | 0.871 |
| 0.0863 | MYBL1 | 0.115 | 0.871 |
| 0.0863 | CDA | 0.024 | 0.871 |
| 0.0863 | BATF3 | 0.018 | 0.871 |
| 0.0863 | CATSPER1 | 0.046 | 0.871 |
| 0.0963 | (Intercept) | −8.080 | 0.861 |
| 0.0963 | CD274 | 0.398 | 0.861 |
| 0.0963 | GBP1 | 0.033 | 0.861 |
| 0.0963 | TBX21 | 0.002 | 0.861 |
| 0.0963 | AIM2 | 0.011 | 0.861 |
| 0.0963 | GZMB | 0.041 | 0.861 |
| 0.0963 | DCBLD2 | 0.137 | 0.861 |
| 0.0963 | AFAP1L2 | 0.013 | 0.861 |
| 0.0963 | FBXO32 | 0.107 | 0.861 |
| 0.0963 | MYBL1 | 0.091 | 0.861 |
| 0.0963 | CDA | 0.018 | 0.861 |
| 0.0963 | BATF3 | 0.001 | 0.861 |
| 0.0963 | CATSPER1 | 0.032 | 0.861 |
| 0.1063 | (Intercept) | −7.225 | 0.842 |
| 0.1063 | CD274 | 0.397 | 0.842 |
| 0.1063 | GBP1 | 0.031 | 0.842 |
| 0.1063 | AIM2 | 0.012 | 0.842 |
| 0.1063 | GZMB | 0.015 | 0.842 |
| 0.1063 | DCBLD2 | 0.127 | 0.842 |
| 0.1063 | FBXO32 | 0.094 | 0.842 |
| 0.1063 | MYBL1 | 0.068 | 0.842 |
| 0.1063 | CDA | 0.011 | 0.842 |
| 0.1063 | CATSPER1 | 0.014 | 0.842 |
| 0.1163 | (Intercept) | −6.436 | 0.842 |
| 0.1163 | CD274 | 0.396 | 0.842 |
| 0.1163 | GBP1 | 0.019 | 0.842 |
| 0.1163 | AIM2 | 0.011 | 0.842 |
| 0.1163 | DCBLD2 | 0.117 | 0.842 |
| 0.1163 | FBXO32 | 0.082 | 0.842 |
| 0.1163 | MYBL1 | 0.043 | 0.842 |
| 0.1163 | CDA | 0.002 | 0.842 |
| 0.1263 | (Intercept) | −5.620 | 0.832 |
| 0.1263 | CD274 | 0.391 | 0.832 |
| 0.1263 | AIM2 | 0.003 | 0.832 |
| 0.1263 | DCBLD2 | 0.103 | 0.832 |
| 0.1263 | FBXO32 | 0.062 | 0.832 |
| 0.1263 | MYBL1 | 0.017 | 0.832 |
| 0.1363 | (Intercept) | −4.948 | 0.812 |
| 0.1363 | CD274 | 0.371 | 0.812 |
| 0.1363 | DCBLD2 | 0.087 | 0.812 |
| 0.1363 | FBXO32 | 0.036 | 0.812 |
| 0.1463 | (Intercept) | −4.322 | 0.812 |
| 0.1463 | CD274 | 0.347 | 0.812 |
| 0.1463 | DCBLD2 | 0.068 | 0.812 |
| 0.1463 | FBXO32 | 0.007 | 0.812 |

TABLE 9A
Results from S3 prediction models based on The
Cancer Genome Atlas (TCGA) lung adenocarcinoma
(LUAD) reverse-phase protein array (RPPA) data.

| Lambda | Predictors | Coefficients | Threshold | Accuracy |
|---|---|---|---|---|
| 0.0279 | (Intercept) | −1.756 | 0.4 | 0.913 |
| | CHK1_pS345 | 0.536 | | |
| | DJ1 | −0.537 | | |
| | ERALPHA | 0.363 | | |
| | GATA3 | −0.322 | | |
| | LCK | 0.094 | | |
| | MIG6 | 1.519 | | |
| | PEA15 | 0.301 | | |
| | PI3KP110ALPHA | 0.051 | | |
| | PKCDELTA_pS664 | 0.181 | | |
| | ANNEXINVII | −0.481 | | |
| | CD20 | −0.245 | | |
| | TIGAR | −0.755 | | |
| | GATA6 | −1.286 | | |
| | BRD4 | 0.460 | | |
| | JAK2 | 1.440 | | |
| | PDL1 | 3.652 | | |
| | PDCD1 | −0.101 | | |
| | TTF1 | 0.060 | | |
| | P63 | −0.002 | | |
| | SYNAPTOPHYSIN | −0.012 | | |

TABLE 9B
Results from S3 prediction models based on The
Cancer Genome Atlas (TCGA) lung adenocarcinoma
(LUAD) reverse-phase protein array (RPPA) data.

| Lambda | Predictors | Coefficients | Threshold | Accuracy |
|---|---|---|---|---|
| 0.0679 | (Intercept) | −1.844 | 0.3 | 0.913 |
| | MIG6 | 0.904 | | |
| | P70S6K1 | 0.089 | | |
| | GATA6 | −0.051 | | |
| | JAK2 | 0.626 | | |
| | PDL1 | 2.862 | | |

TABLE 10
Accuracy of S3 prediction models based on The
Cancer Genome Atlas (TCGA)
lung adenocarcinoma (LUAD) reverse-phase protein
array (RPPA) data with varying lambda values.
The five-feature model is in bold. Each model is preceded by
the Intercept corresponding to the model.

| Lambda | Predictors | Coefficients | Accuracy |
|---|---|---|---|
| 0.028 | (Intercept) | −1.756 | 0.913 |
| 0.028 | CHK1_pS345 | 0.536 | 0.913 |
| 0.028 | DJ1 | −0.537 | 0.913 |
| 0.028 | ERALPHA | 0.363 | 0.913 |
| 0.028 | GATA3 | −0.322 | 0.913 |
| 0.028 | LCK | 0.094 | 0.913 |
| 0.028 | MIG6 | 1.519 | 0.913 |
| 0.028 | PEA15 | 0.301 | 0.913 |
| 0.028 | PI3KP110ALPHA | 0.051 | 0.913 |
| 0.028 | PKCDELTA_pS664 | 0.181 | 0.913 |
| 0.028 | ANNEXINVII | −0.481 | 0.913 |
| 0.028 | CD20 | −0.245 | 0.913 |
| 0.028 | TIGAR | −0.755 | 0.913 |
| 0.028 | GATA6 | −1.286 | 0.913 |
| 0.028 | BRD4 | 0.460 | 0.913 |
| 0.028 | JAK2 | 1.440 | 0.913 |
| 0.028 | PDL1 | 3.652 | 0.913 |
| 0.028 | PDCD1 | −0.101 | 0.913 |
| 0.028 | TTF1 | 0.060 | 0.913 |
| 0.028 | P63 | −0.002 | 0.913 |
| 0.028 | SYNAPTOPHYSIN | −0.012 | 0.913 |
| 0.038 | (Intercept) | −1.737 | 0.913 |
| 0.038 | DJ1 | −0.406 | 0.913 |
| 0.038 | ERALPHA | 0.246 | 0.913 |
| 0.038 | GATA3 | −0.006 | 0.913 |
| 0.038 | MIG6 | 1.414 | 0.913 |
| 0.038 | PEA15 | 0.043 | 0.913 |
| 0.038 | ANNEXINVII | −0.180 | 0.913 |
| 0.038 | CD20 | −0.273 | 0.913 |
| 0.038 | TIGAR | −0.443 | 0.913 |
| 0.038 | GATA6 | −1.063 | 0.913 |
| 0.038 | BRD4 | 0.193 | 0.913 |
| 0.038 | JAK2 | 1.377 | 0.913 |
| 0.038 | PDL1 | 3.283 | 0.913 |
| 0.038 | PDCD1 | −0.030 | 0.913 |
| 0.038 | TTF1 | 0.039 | 0.913 |
| 0.048 | (Intercept) | −1.799 | 0.913 |
| 0.048 | DJ1 | −0.186 | 0.913 |
| 0.048 | ERALPHA | 0.130 | 0.913 |
| 0.048 | MIG6 | 1.202 | 0.913 |
| 0.048 | P70S6K1 | 0.130 | 0.913 |
| 0.048 | CD20 | −0.110 | 0.913 |
| 0.048 | TIGAR | −0.217 | 0.913 |
| 0.048 | GATA6 | −0.749 | 0.913 |
| 0.048 | JAK2 | 1.113 | 0.913 |
| 0.048 | PDL1 | 3.087 | 0.913 |
| 0.048 | TTF1 | 0.013 | 0.913 |
| 0.058 | (Intercept) | −1.887 | 0.913 |
| 0.058 | DJ1 | −0.021 | 0.913 |
| 0.058 | ERALPHA | 0.012 | 0.913 |
| 0.058 | MIG6 | 1.046 | 0.913 |
| 0.058 | P70S6K1 | 0.127 | 0.913 |
| 0.058 | TIGAR | −0.011 | 0.913 |
| 0.058 | GATA6 | −0.361 | 0.913 |
| 0.058 | JAK2 | 0.852 | 0.913 |
| 0.058 | PDL1 | 2.993 | 0.913 |
| 0.068 | (Intercept) | −1.844 | 0.913 |
| 0.068 | MIG6 | 0.904 | 0.913 |
| 0.068 | P70S6K1 | 0.089 | 0.913 |
| 0.068 | GATA6 | −0.051 | 0.913 |
| 0.068 | JAK2 | 0.626 | 0.913 |
| 0.068 | PDL1 | 2.862 | 0.913 |
| 0.078 | (Intercept) | −1.790 | 0.891 |
| 0.078 | MIG6 | 0.785 | 0.891 |
| 0.078 | P70S6K1 | 0.019 | 0.891 |
| 0.078 | JAK2 | 0.442 | 0.891 |
| 0.078 | PDL1 | 2.694 | 0.891 |
| 0.088 | (Intercept) | −1.738 | 0.891 |
| 0.088 | MIG6 | 0.645 | 0.891 |
| 0.088 | JAK2 | 0.257 | 0.891 |
| 0.088 | PDL1 | 2.537 | 0.891 |
| 0.098 | (Intercept) | −1.691 | 0.891 |
| 0.098 | MIG6 | 0.501 | 0.891 |
| 0.098 | JAK2 | 0.074 | 0.891 |
| 0.098 | PDL1 | 2.395 | 0.891 |
| 0.108 | (Intercept) | −1.637 | 0.870 |
| 0.108 | MIG6 | 0.363 | 0.870 |
| 0.108 | PDL1 | 2.249 | 0.870 |
| 0.118 | (Intercept) | −1.579 | 0.783 |
| 0.118 | MIG6 | 0.230 | 0.783 |
| 0.118 | PDL1 | 2.102 | 0.783 |
| 0.128 | (Intercept) | −1.523 | 0.761 |
| 0.128 | MIG6 | 0.098 | 0.761 |
| 0.128 | PDL1 | 1.962 | 0.761 |
| 0.138 | (Intercept) | −1.475 | 0.761 |
| 0.138 | PDL1 | 1.818 | 0.761 |
| 0.148 | (Intercept) | −1.445 | 0.761 |
| 0.148 | PDL1 | 1.644 | 0.761 |

TABLE 11A
Results from S4 prediction models based on The
Cancer Genome Atlas (TCGA) lung
adenocarcinoma (LUAD) gene expression data.

| Lambda | Predictors | Coefficients | Threshold | Accuracy |
|---|---|---|---|---|
| 0.0153 | (Intercept) | −7.7628 | 0.5 | 0.851 |
| | LOC100190940 | 0.0524 | | |
| | KCNU1 | 0.0962 | | |
| | ZMAT4 | 0.0841 | | |
| | SLC38A8 | 0.1959 | | |
| | HOXD13 | 0.2698 | | |
| | PCSK1 | 0.0400 | | |
| | UGT3A1 | 0.0234 | | |
| | KLK14 | 0.1258 | | |
| | HEPACAM2 | 0.0366 | | |
| | CPS1 | 0.0494 | | |
| | CALB1 | 0.0453 | | |
| | AKR1C4 | 0.1506 | | |
| | F2 | 0.0372 | | |
| | MLLT11 | 0.2837 | | |
| | INSL4 | 0.0801 | | |
| | HOXD11 | 0.0609 | | |
| | AKR1C2 | 0.0840 | | |
| | F7 | 0.0017 | | |
| | WDR72 | 0.0244 | | |
| | UCHL1 | 0.0035 | | |
| | POPDC3 | 0.0330 | | |
| | CSAG2 | 0.0804 | | |
| | C20orf70 | 0.0508 | | |
| | GNG4 | 0.0279 | | |
| | C12orf39 | 0.2302 | | |
| | C12orf56 | 0.0109 | | |
| | IGF2BP1 | 0.0818 | | |

TABLE 11B
Results from S4 prediction models based on The
Cancer Genome Atlas (TCGA) lung
adenocarcinoma (LUAD) gene expression data.

| Lambda | Predictors | Coefficients | Threshold | Accuracy |
|---|---|---|---|---|
| 0.215 | (Intercept) | −1.326 | 0.3 | 0.673 |
| | CALCA | 0.002 | | |
| | HOXD13 | 0.056 | | |
| | AKR1C4 | 0.006 | | |
| | MLLT11 | 0.038 | | |
| | PAH | 0.010 | | |

TABLE 12
Accuracy of S4 prediction models based on
The Cancer Genome Atlas (TCGA)
lung adenocarcinoma (LUAD) gene expression
data with varying lambda values. The five-feature
model is in bold. Each model is preceded by the
Intercept corresponding to the model.

| Lambda | Predictors | Coefficients | Accuracy |
|---|---|---|---|
| 0.015 | (Intercept) | −7.763 | 0.851 |
| 0.015 | LOC100190940 | 0.052 | 0.851 |
| 0.015 | KCNU1 | 0.096 | 0.851 |
| 0.015 | ZMAT4 | 0.084 | 0.851 |
| 0.015 | SLC38A8 | 0.196 | 0.851 |
| 0.015 | HOXD13 | 0.270 | 0.851 |
| 0.015 | PCSK1 | 0.040 | 0.851 |
| 0.015 | UGT3A1 | 0.023 | 0.851 |
| 0.015 | KLK14 | 0.126 | 0.851 |
| 0.015 | HEPACAM2 | 0.037 | 0.851 |
| 0.015 | CPS1 | 0.049 | 0.851 |
| 0.015 | CALB1 | 0.045 | 0.851 |
| 0.015 | AKRIC4 | 0.151 | 0.851 |
| 0.015 | F2 | 0.037 | 0.851 |
| 0.015 | MLLT11 | 0.284 | 0.851 |
| 0.015 | INSL4 | 0.080 | 0.851 |
| 0.015 | HOXD11 | 0.061 | 0.851 |
| 0.015 | AKRIC2 | 0.084 | 0.851 |
| 0.015 | F7 | 0.002 | 0.851 |
| 0.015 | WDR72 | 0.024 | 0.851 |
| 0.015 | UCHL1 | 0.003 | 0.851 |
| 0.015 | POPDC3 | 0.033 | 0.851 |
| 0.015 | CSAG2 | 0.080 | 0.851 |
| 0.015 | C20orf70 | 0.051 | 0.851 |
| 0.015 | GNG4 | 0.028 | 0.851 |
| 0.015 | C12orf39 | 0.230 | 0.851 |
| 0.015 | C12orf56 | 0.011 | 0.851 |
| 0.015 | IGF2BP1 | 0.082 | 0.851 |
| 0.025 | (Intercept) | −6.735 | 0.832 |
| 0.025 | LOC100190940 | 0.038 | 0.832 |
| 0.025 | KCNU1 | 0.079 | 0.832 |
| 0.025 | ZMAT4 | 0.053 | 0.832 |
| 0.025 | SLC38A8 | 0.152 | 0.832 |
| 0.025 | HOXD13 | 0.243 | 0.832 |
| 0.025 | PCSK1 | 0.029 | 0.832 |
| 0.025 | UGT3A1 | 0.014 | 0.832 |
| 0.025 | KLK14 | 0.105 | 0.832 |
| 0.025 | HEPACAM2 | 0.034 | 0.832 |
| 0.025 | CPS1 | 0.058 | 0.832 |
| 0.025 | CALB1 | 0.011 | 0.832 |
| 0.025 | AKRIC4 | 0.139 | 0.832 |
| 0.025 | GLDC | 0.030 | 0.832 |
| 0.025 | F2 | 0.020 | 0.832 |
| 0.025 | MLLT11 | 0.259 | 0.832 |
| 0.025 | INSL4 | 0.064 | 0.832 |
| 0.025 | HOXD11 | 0.038 | 0.832 |
| 0.025 | AKRIC2 | 0.059 | 0.832 |
| 0.025 | WDR72 | 0.029 | 0.832 |
| 0.025 | POPDC3 | 0.026 | 0.832 |
| 0.025 | CSAG2 | 0.046 | 0.832 |
| 0.025 | C20orf70 | 0.037 | 0.832 |
| 0.025 | GNG4 | 0.018 | 0.832 |
| 0.025 | C12orf39 | 0.162 | 0.832 |
| 0.025 | IGF2BP1 | 0.071 | 0.832 |
| 0.035 | (Intercept) | −6.042 | 0.822 |
| 0.035 | LOC100190940 | 0.032 | 0.822 |
| 0.035 | KCNU1 | 0.063 | 0.822 |
| 0.035 | ZMAT4 | 0.033 | 0.822 |
| 0.035 | SLC38A8 | 0.126 | 0.822 |
| 0.035 | HOXD13 | 0.229 | 0.822 |
| 0.035 | PCSK1 | 0.022 | 0.822 |
| 0.035 | UGT3A1 | 0.005 | 0.822 |
| 0.035 | KLK14 | 0.089 | 0.822 |
| 0.035 | HEPACAM2 | 0.032 | 0.822 |
| 0.035 | CPS1 | 0.064 | 0.822 |
| 0.035 | AKRIC4 | 0.133 | 0.822 |
| 0.035 | GLDC | 0.050 | 0.822 |
| 0.035 | F2 | 0.013 | 0.822 |
| 0.035 | MLLT11 | 0.244 | 0.822 |
| 0.035 | INSL4 | 0.052 | 0.822 |
| 0.035 | HOXD11 | 0.022 | 0.822 |
| 0.035 | AKRIC2 | 0.041 | 0.822 |
| 0.035 | WDR72 | 0.028 | 0.822 |
| 0.035 | POPDC3 | 0.018 | 0.822 |
| 0.035 | CSAG2 | 0.023 | 0.822 |
| 0.035 | C20orf70 | 0.023 | 0.822 |
| 0.035 | GNG4 | 0.011 | 0.822 |
| 0.035 | C12orf39 | 0.116 | 0.822 |
| 0.035 | IGF2BP1 | 0.059 | 0.822 |
| 0.045 | (Intercept) | −5.513 | 0.802 |
| 0.045 | LOC100190940 | 0.028 | 0.802 |
| 0.045 | KCNU1 | 0.048 | 0.802 |
| 0.045 | ZMAT4 | 0.019 | 0.802 |
| 0.045 | SLC38A8 | 0.108 | 0.802 |
| 0.045 | HOXD13 | 0.220 | 0.802 |
| 0.045 | PCSK1 | 0.016 | 0.802 |
| 0.045 | KLK14 | 0.075 | 0.802 |
| 0.045 | HEPACAM2 | 0.030 | 0.802 |
| 0.045 | CPS1 | 0.069 | 0.802 |
| 0.045 | AKRIC4 | 0.131 | 0.802 |
| 0.045 | GLDC | 0.063 | 0.802 |
| 0.045 | F2 | 0.009 | 0.802 |
| 0.045 | MLLT11 | 0.234 | 0.802 |
| 0.045 | INSL4 | 0.041 | 0.802 |
| 0.045 | HOXD11 | 0.009 | 0.802 |
| 0.045 | AKRIC2 | 0.028 | 0.802 |
| 0.045 | WDR72 | 0.026 | 0.802 |
| 0.045 | POPDC3 | 0.010 | 0.802 |
| 0.045 | CSAG2 | 0.005 | 0.802 |
| 0.045 | C20orf70 | 0.012 | 0.802 |
| 0.045 | GNG4 | 0.006 | 0.802 |
| 0.045 | C12orf39 | 0.084 | 0.802 |
| 0.045 | IGF2BP1 | 0.049 | 0.802 |
| 0.055 | (Intercept) | −5.050 | 0.812 |
| 0.055 | LOC100190940 | 0.026 | 0.812 |
| 0.055 | KCNU1 | 0.037 | 0.812 |
| 0.055 | ZMAT4 | 0.009 | 0.812 |
| 0.055 | SLC38A8 | 0.096 | 0.812 |
| 0.055 | HOXD13 | 0.212 | 0.812 |
| 0.055 | PCSK1 | 0.012 | 0.812 |
| 0.055 | KLK14 | 0.061 | 0.812 |
| 0.055 | HEPACAM2 | 0.025 | 0.812 |
| 0.055 | CPS1 | 0.073 | 0.812 |
| 0.055 | AKRIC4 | 0.130 | 0.812 |
| 0.055 | GLDC | 0.071 | 0.812 |
| 0.055 | F2 | 0.007 | 0.812 |
| 0.055 | MLLT11 | 0.224 | 0.812 |
| 0.055 | INSL4 | 0.030 | 0.812 |
| 0.055 | AKRIC2 | 0.017 | 0.812 |
| 0.055 | WDR72 | 0.023 | 0.812 |
| 0.055 | POPDC3 | 0.001 | 0.812 |
| 0.055 | C20orf70 | 0.001 | 0.812 |
| 0.055 | GNG4 | 0.001 | 0.812 |
| 0.055 | C12orf39 | 0.059 | 0.812 |
| 0.055 | IGF2BP1 | 0.040 | 0.812 |
| 0.065 | (Intercept) | −4.645 | 0.802 |
| 0.065 | LOC100190940 | 0.025 | 0.802 |
| 0.065 | KCNU1 | 0.030 | 0.802 |
| 0.065 | SLC38A8 | 0.087 | 0.802 |
| 0.065 | HOXD13 | 0.202 | 0.802 |
| 0.065 | PCSK1 | 0.007 | 0.802 |
| 0.065 | KLK14 | 0.046 | 0.802 |
| 0.065 | HEPACAM2 | 0.020 | 0.802 |
| 0.065 | CPS1 | 0.076 | 0.802 |
| 0.065 | AKRIC4 | 0.130 | 0.802 |
| 0.065 | GLDC | 0.074 | 0.802 |
| 0.065 | F2 | 0.008 | 0.802 |
| 0.065 | MLLT11 | 0.212 | 0.802 |
| 0.065 | INSL4 | 0.016 | 0.802 |
| 0.065 | AKR1C2 | 0.006 | 0.802 |
| 0.065 | WDR72 | 0.019 | 0.802 |
| 0.065 | C12orf39 | 0.037 | 0.802 |
| 0.065 | IGF2BP1 | 0.031 | 0.802 |
| 0.075 | (Intercept) | −4.296 | 0.772 |
| 0.075 | LOC100190940 | 0.024 | 0.772 |
| 0.075 | KCNU1 | 0.022 | 0.772 |
| 0.075 | SLC38A8 | 0.078 | 0.772 |
| 0.075 | HOXD13 | 0.193 | 0.772 |
| 0.075 | PCSK1 | 0.004 | 0.772 |
| 0.075 | KLK14 | 0.030 | 0.772 |
| 0.075 | HEPACAM2 | 0.013 | 0.772 |
| 0.075 | CPS1 | 0.078 | 0.772 |
| 0.075 | AKRIC4 | 0.129 | 0.772 |
| 0.075 | GLDC | 0.074 | 0.772 |
| 0.075 | F2 | 0.010 | 0.772 |
| 0.075 | MLLT11 | 0.199 | 0.772 |
| 0.075 | PAH | 0.002 | 0.772 |
| 0.075 | INSL4 | 0.003 | 0.772 |
| 0.075 | WDR72 | 0.016 | 0.772 |
| 0.075 | C12orf39 | 0.017 | 0.772 |
| 0.075 | IGF2BP1 | 0.023 | 0.772 |
| 0.085 | (Intercept) | −4.002 | 0.772 |
| 0.085 | LOC100190940 | 0.023 | 0.772 |
| 0.085 | KCNU1 | 0.013 | 0.772 |
| 0.085 | SLC38A8 | 0.067 | 0.772 |
| 0.085 | HOXD13 | 0.183 | 0.772 |
| 0.085 | PCSK1 | 0.004 | 0.772 |
| 0.085 | KLK14 | 0.020 | 0.772 |
| 0.085 | HEPACAM2 | 0.008 | 0.772 |
| 0.085 | CPS1 | 0.076 | 0.772 |
| 0.085 | AKRIC4 | 0.121 | 0.772 |
| 0.085 | GLDC | 0.071 | 0.772 |
| 0.085 | F2 | 0.007 | 0.772 |
| 0.085 | MLLT11 | 0.184 | 0.772 |
| 0.085 | PAH | 0.008 | 0.772 |
| 0.085 | WDR72 | 0.011 | 0.772 |
| 0.085 | IGF2BP1 | 0.017 | 0.772 |
| 0.095 | (Intercept) | −3.712 | 0.782 |
| 0.095 | LOC100190940 | 0.024 | 0.782 |
| 0.095 | KCNU1 | 0.006 | 0.782 |
| 0.095 | SLC38A8 | 0.061 | 0.782 |
| 0.095 | HOXD13 | 0.174 | 0.782 |
| 0.095 | PCSK1 | 0.004 | 0.782 |
| 0.095 | KLK14 | 0.012 | 0.782 |
| 0.095 | HEPACAM2 | 0.003 | 0.782 |
| 0.095 | CPS1 | 0.070 | 0.782 |
| 0.095 | AKR1C4 | 0.113 | 0.782 |
| 0.095 | GLDC | 0.065 | 0.782 |
| 0.095 | F2 | 0.000 | 0.782 |
| 0.095 | MLLT11 | 0.171 | 0.782 |
| 0.095 | PAH | 0.012 | 0.782 |
| 0.095 | WDR72 | 0.005 | 0.782 |
| 0.095 | IGF2BP1 | 0.011 | 0.782 |
| 0.105 | (Intercept) | −3.437 | 0.772 |
| 0.105 | LOC100190940 | 0.024 | 0.772 |
| 0.105 | KCNU1 | 0.001 | 0.772 |
| 0.105 | SLC38A8 | 0.056 | 0.772 |
| 0.105 | HOXD13 | 0.165 | 0.772 |
| 0.105 | PCSK1 | 0.003 | 0.772 |
| 0.105 | KLK14 | 0.005 | 0.772 |
| 0.105 | CPS1 | 0.065 | 0.772 |
| 0.105 | AKRIC4 | 0.105 | 0.772 |
| 0.105 | GLDC | 0.060 | 0.772 |
| 0.105 | MLLT11 | 0.159 | 0.772 |
| 0.105 | PAH | 0.015 | 0.772 |
| 0.105 | IGF2BP1 | 0.004 | 0.772 |
| 0.115 | (Intercept) | −3.205 | 0.762 |
| 0.115 | LOC100190940 | 0.023 | 0.762 |
| 0.115 | SLC38A8 | 0.048 | 0.762 |
| 0.115 | HOXD13 | 0.155 | 0.762 |
| 0.115 | PCSK1 | 0.002 | 0.762 |
| 0.115 | CPS1 | 0.059 | 0.762 |
| 0.115 | AKRIC4 | 0.095 | 0.762 |
| 0.115 | GLDC | 0.053 | 0.762 |
| 0.115 | MLLT11 | 0.148 | 0.762 |
| 0.115 | PAH | 0.016 | 0.762 |
| 0.125 | (Intercept) | −2.980 | 0.743 |
| 0.125 | LOC100190940 | 0.021 | 0.743 |
| 0.125 | SLC38A8 | 0.036 | 0.743 |
| 0.125 | CALCA | 0.003 | 0.743 |
| 0.125 | HOXD13 | 0.144 | 0.743 |
| 0.125 | CPS1 | 0.053 | 0.743 |
| 0.125 | AKR1C4 | 0.085 | 0.743 |
| 0.125 | GLDC | 0.045 | 0.743 |
| 0.125 | MLLT11 | 0.136 | 0.743 |
| 0.125 | PAH | 0.016 | 0.743 |
| 0.135 | (Intercept) | −2.763 | 0.743 |
| 0.135 | LOC100190940 | 0.018 | 0.743 |
| 0.135 | SLC38A8 | 0.024 | 0.743 |
| 0.135 | CALCA | 0.005 | 0.743 |
| 0.135 | HOXD13 | 0.133 | 0.743 |
| 0.135 | CPS1 | 0.046 | 0.743 |
| 0.135 | AKR1C4 | 0.075 | 0.743 |
| 0.135 | GLDC | 0.037 | 0.743 |
| 0.135 | MLLT11 | 0.124 | 0.743 |
| 0.135 | PAH | 0.016 | 0.743 |
| 0.145 | (Intercept) | −2.556 | 0.733 |
| 0.145 | LOC100190940 | 0.016 | 0.733 |
| 0.145 | SLC38A8 | 0.012 | 0.733 |
| 0.145 | CALCA | 0.008 | 0.733 |
| 0.145 | HOXD13 | 0.123 | 0.733 |
| 0.145 | CPS1 | 0.040 | 0.733 |
| 0.145 | AKRIC4 | 0.066 | 0.733 |
| 0.145 | GLDC | 0.029 | 0.733 |
| 0.145 | MLLT11 | 0.112 | 0.733 |
| 0.145 | PAH | 0.016 | 0.733 |
| 0.155 | (Intercept) | −2.358 | 0.713 |
| 0.155 | LOC100190940 | 0.014 | 0.713 |
| 0.155 | SLC38A8 | 0.000 | 0.713 |
| 0.155 | CALCA | 0.010 | 0.713 |
| 0.155 | HOXD13 | 0.113 | 0.713 |
| 0.155 | CPS1 | 0.034 | 0.713 |
| 0.155 | AKR1C4 | 0.057 | 0.713 |
| 0.155 | GLDC | 0.021 | 0.713 |
| 0.155 | MLLT11 | 0.101 | 0.713 |
| 0.155 | PAH | 0.016 | 0.713 |
| 0.165 | (Intercept) | −2.165 | 0.703 |
| 0.165 | LOC100190940 | 0.011 | 0.703 |
| 0.165 | CALCA | 0.010 | 0.703 |
| 0.165 | HOXD13 | 0.104 | 0.703 |
| 0.165 | CPS1 | 0.028 | 0.703 |
| 0.165 | AKR1C4 | 0.049 | 0.703 |
| 0.165 | GLDC | 0.014 | 0.703 |
| 0.165 | MLLT11 | 0.090 | 0.703 |
| 0.165 | PAH | 0.015 | 0.703 |
| 0.175 | (Intercept) | −1.978 | 0.683 |
| 0.175 | LOC100190940 | 0.008 | 0.683 |
| 0.175 | CALCA | 0.009 | 0.683 |
| 0.175 | HOXD13 | 0.094 | 0.683 |
| 0.175 | CPS1 | 0.022 | 0.683 |
| 0.175 | AKRIC4 | 0.041 | 0.683 |
| 0.175 | GLDC | 0.007 | 0.683 |
| 0.175 | MLLT11 | 0.080 | 0.683 |
| 0.175 | PAH | 0.015 | 0.683 |
| 0.185 | (Intercept) | −1.797 | 0.673 |
| 0.185 | LOC100190940 | 0.005 | 0.673 |
| 0.185 | CALCA | 0.009 | 0.673 |
| 0.185 | HOXD13 | 0.085 | 0.673 |
| 0.185 | CPS1 | 0.017 | 0.673 |
| 0.185 | AKRIC4 | 0.033 | 0.673 |
| 0.185 | MLLT11 | 0.070 | 0.673 |
| 0.185 | PAH | 0.014 | 0.673 |
| 0.195 | (Intercept) | −1.634 | 0.673 |
| 0.195 | LOC100190940 | 0.001 | 0.673 |
| 0.195 | CALCA | 0.008 | 0.673 |
| 0.195 | HOXD13 | 0.076 | 0.673 |
| 0.195 | CPS1 | 0.010 | 0.673 |
| 0.195 | AKR1C4 | 0.025 | 0.673 |
| 0.195 | MLLT11 | 0.059 | 0.673 |
| 0.195 | PAH | 0.013 | 0.673 |
| 0.205 | (Intercept) | −1.472 | 0.673 |
| 0.205 | CALCA | 0.006 | 0.673 |
| 0.205 | HOXD13 | 0.066 | 0.673 |
| 0.205 | CPS1 | 0.004 | 0.673 |
| 0.205 | AKRIC4 | 0.016 | 0.673 |
| 0.205 | MLLT11 | 0.048 | 0.673 |
| 0.205 | PAH | 0.012 | 0.673 |
| 0.215 | (Intercept) | −1.326 | 0.673 |
| 0.215 | CALCA | 0.002 | 0.673 |
| 0.215 | HOXD13 | 0.056 | 0.673 |
| 0.215 | AKR1C4 | 0.006 | 0.673 |
| 0.215 | MLLT11 | 0.038 | 0.673 |
| 0.215 | PAH | 0.010 | 0.673 |
| 0.225 | (Intercept) | −1.192 | 0.673 |
| 0.225 | HOXD13 | 0.045 | 0.673 |
| 0.225 | MLLT11 | 0.026 | 0.673 |
| 0.225 | PAH | 0.003 | 0.673 |
| 0.235 | (Intercept) | −1.038 | 0.673 |
| 0.235 | HOXD13 | 0.031 | 0.673 |
| 0.235 | MLLT11 | 0.007 | 0.673 |
| 0.245 | (Intercept) | −0.970 | 0.673 |
| 0.245 | HOXD13 | 0.011 | 0.673 |
| 0.255 | (Intercept) | −0.960 | 0.673 |
| 0.255 | LOC441177 | 0.000 | 0.673 |
| 0.265 | (Intercept) | −0.960 | 0.673 |
| 0.265 | LOC441177 | 0.000 | 0.673 |
| 0.275 | (Intercept) | −0.960 | 0.673 |
| 0.275 | LOC441177 | 0.000 | 0.673 |
| 0.285 | (Intercept) | −0.960 | 0.673 |
| 0.285 | LOC441177 | 0.000 | 0.673 |
| 0.295 | (Intercept) | −0.960 | 0.673 |
| 0.295 | LOC441177 | 0.000 | 0.673 |

TABLE 13A
Results from S4 prediction models based on The Cancer Genome Atlas (TCGA) lung
adenocarcinoma (LUAD) reverse-phase protein array (RPPA) data.

| Lambda | Predictors | Coefficients | Threshold | Accuracy |
|---|---|---|---|---|
| 0.0283 | (Intercept) | 1.621 | 0.5 | 0.913 |
| | AMPKALPHA | 1.163 | | |
| | BIM | 1.147 | | |
| | CASPASE7CLEAVEDD198 | 0.027 | | |
| | CAVEOLIN1 | −0.145 | | |
| | CYCLINB1 | 0.610 | | |
| | JNK2 | −0.480 | | |
| | MIG6 | −1.449 | | |
| | MTOR_pS2448 | −0.428 | | |
| | NCADHERIN | 0.517 | | |
| | P38MAPK | −0.125 | | |
| | PEA15 | −1.106 | | |
| | PKCALPHA_pS657 | −0.290 | | |
| | VEGFR2 | −0.098 | | |
| | YAP_pS127 | −0.393 | | |
| | P90RSK | −0.049 | | |
| | TIGAR | 0.263 | | |
| | TFRC | 0.017 | | |
| | ACETYLATUBULINLYS40 | −0.116 | | |
| | ANNEXIN1 | −0.374 | | |
| | MSH6 | 0.447 | | |
| | NRF2 | 0.558 | | |
| | TTF1 | −0.316 | | |
| | NAPSINA | −0.258 | | |
| | SYNAPTOPHYSIN | 0.054 | | |

TABLE 13B
Results from S4 prediction models based on
The Cancer Genome Atlas (TCGA) lung adenocarcinoma
(LUAD) reverse-phase protein array (RPPA) data.

| Lambda | Predictors | Coefficients | Threshold | Accuracy |
|---|---|---|---|---|
| 0.168 | (Intercept) | −0.656 | 0.4 | 0.739 |
| | BIM | 0.477 | | |
| | CAVEOLIN1 | −0.009 | | |
| | FOXM1 | 0.036 | | |
| | PKCPANBETAII_pS660 | −0.054 | | |
| | NRF2 | 0.342 | | |

TABLE 14
Accuracy of S3 prediction models based on The
Cancer Genome Atlas (TCGA) lung
adenocarcinoma (LUAD) reverse-phase protein
array (RPPA) data with varying lambda values.
The five-feature model is in bold. Each model is preceded
by the Intercept corresponding to the model.

| Lambda | Predictors | Coefficients | Accuracy |
|---|---|---|---|
| 0.028 | (Intercept) | 1.621 | 0.913 |
| 0.028 | AMPKALPHA | 1.163 | 0.913 |
| 0.028 | BIM | 1.147 | 0.913 |
| 0.028 | CASPASE7CLEAVEDD198 | 0.027 | 0.913 |
| 0.028 | CAVEOLIN1 | −0.145 | 0.913 |
| 0.028 | CYCLINB1 | 0.610 | 0.913 |
| 0.028 | JNK2 | −0.480 | 0.913 |
| 0.028 | MIG6 | −1.449 | 0.913 |
| 0.028 | MTOR_pS2448 | −0.428 | 0.913 |
| 0.028 | NCADHERIN | 0.517 | 0.913 |
| 0.028 | P38MAPK | −0.125 | 0.913 |
| 0.028 | PEA15 | −1.106 | 0.913 |
| 0.028 | PKCALPHA_pS657 | −0.290 | 0.913 |
| 0.028 | VEGFR2 | −0.098 | 0.913 |
| 0.028 | YAP_pS127 | −0.393 | 0.913 |
| 0.028 | P90RSK | −0.049 | 0.913 |
| 0.028 | TIGAR | 0.263 | 0.913 |
| 0.028 | TFRC | 0.017 | 0.913 |
| 0.028 | ACETYLATUBULINLYS40 | −0.116 | 0.913 |
| 0.028 | ANNEXIN1 | −0.374 | 0.913 |
| 0.028 | MSH6 | 0.447 | 0.913 |
| 0.028 | NRF2 | 0.558 | 0.913 |
| 0.028 | TTF1 | −0.316 | 0.913 |
| 0.028 | NAPSINA | −0.258 | 0.913 |
| 0.028 | SYNAPTOPHYSIN | 0.054 | 0.913 |
| 0.038 | (Intercept) | 1.310 | 0.913 |
| 0.038 | AMPKALPHA | 0.838 | 0.913 |
| 0.038 | BIM | 1.050 | 0.913 |
| 0.038 | CASPASE7CLEAVEDD198 | 0.012 | 0.913 |
| 0.038 | CAVEOLIN1 | −0.091 | 0.913 |
| 0.038 | CYCLINB1 | 0.582 | 0.913 |
| 0.038 | JNK2 | −0.423 | 0.913 |
| 0.038 | MIG6 | −0.923 | 0.913 |
| 0.038 | MTOR_pS2448 | −0.181 | 0.913 |
| 0.038 | NCADHERIN | 0.378 | 0.913 |
| 0.038 | P38MAPK | −0.086 | 0.913 |
| 0.038 | PEA15 | −0.717 | 0.913 |
| 0.038 | PKCALPHA_pS657 | −0.314 | 0.913 |
| 0.038 | VEGFR2 | −0.017 | 0.913 |
| 0.038 | YAP_pS127 | −0.384 | 0.913 |
| 0.038 | TIGAR | 0.260 | 0.913 |
| 0.038 | TFRC | 0.025 | 0.913 |
| 0.038 | ACETYLATUBULINLYS40 | −0.018 | 0.913 |
| 0.038 | ANNEXIN1 | −0.409 | 0.913 |
| 0.038 | MSH6 | 0.234 | 0.913 |
| 0.038 | NRF2 | 0.711 | 0.913 |
| 0.038 | TTF1 | −0.279 | 0.913 |
| 0.038 | NAPSINA | −0.144 | 0.913 |
| 0.048 | (Intercept) | 1.050 | 0.913 |
| 0.048 | AMPKALPHA | 0.582 | 0.913 |
| 0.048 | BIM | 0.971 | 0.913 |
| 0.048 | CASPASE7CLEAVEDD198 | 0.001 | 0.913 |
| 0.048 | CAVEOLIN1 | −0.063 | 0.913 |
| 0.048 | CYCLINB1 | 0.553 | 0.913 |
| 0.048 | JNK2 | −0.337 | 0.913 |
| 0.048 | MIG6 | −0.504 | 0.913 |
| 0.048 | MTOR_pS2448 | −0.010 | 0.913 |
| 0.048 | NCADHERIN | 0.279 | 0.913 |
| 0.048 | P38MAPK | −0.069 | 0.913 |
| 0.048 | PEA15 | −0.436 | 0.913 |
| 0.048 | PKCALPHA_pS657 | −0.318 | 0.913 |
| 0.048 | YAP_pS127 | −0.350 | 0.913 |
| 0.048 | TIGAR | 0.241 | 0.913 |
| 0.048 | TFRC | 0.029 | 0.913 |
| 0.048 | ANNEXIN1 | −0.415 | 0.913 |
| 0.048 | MSH6 | 0.103 | 0.913 |
| 0.048 | NRF2 | 0.780 | 0.913 |
| 0.048 | TTF1 | −0.250 | 0.913 |
| 0.048 | NAPSINA | −0.046 | 0.913 |
| 0.058 | (Intercept) | 0.828 | 0.913 |
| 0.058 | AMPKALPHA | 0.377 | 0.913 |
| 0.058 | BIM | 0.899 | 0.913 |
| 0.058 | CAVEOLIN1 | −0.044 | 0.913 |
| 0.058 | CYCLINB1 | 0.507 | 0.913 |
| 0.058 | JNK2 | −0.218 | 0.913 |
| 0.058 | MIG6 | −0.175 | 0.913 |
| 0.058 | NCADHERIN | 0.197 | 0.913 |
| 0.058 | P38MAPK | −0.060 | 0.913 |
| 0.058 | PEA15 | −0.224 | 0.913 |
| 0.058 | PKCALPHA_pS657 | −0.326 | 0.913 |
| 0.058 | YAP_pS127 | −0.303 | 0.913 |
| 0.058 | TIGAR | 0.203 | 0.913 |
| 0.058 | TFRC | 0.028 | 0.913 |
| 0.058 | ANNEXIN1 | −0.417 | 0.913 |
| 0.058 | NRF2 | 0.805 | 0.913 |
| 0.058 | TTF1 | −0.222 | 0.913 |
| 0.068 | (Intercept) | 0.670 | 0.891 |
| 0.068 | AMPKALPHA | 0.177 | 0.891 |
| 0.068 | BIM | 0.807 | 0.891 |
| 0.068 | CAVEOLIN1 | −0.035 | 0.891 |
| 0.068 | CYCLINB1 | 0.439 | 0.891 |
| 0.068 | JNK2 | −0.089 | 0.891 |
| 0.068 | NCADHERIN | 0.154 | 0.891 |
| 0.068 | P38MAPK | −0.035 | 0.891 |
| 0.068 | PEA15 | −0.034 | 0.891 |
| 0.068 | PKCALPHA_pS657 | −0.334 | 0.891 |
| 0.068 | YAP_pS127 | −0.248 | 0.891 |
| 0.068 | TIGAR | 0.152 | 0.891 |
| 0.068 | TFRC | 0.020 | 0.891 |
| 0.068 | ANNEXIN1 | −0.408 | 0.891 |
| 0.068 | DUSP4 | 0.020 | 0.891 |
| 0.068 | NRF2 | 0.797 | 0.891 |
| 0.068 | TTF1 | −0.198 | 0.891 |
| 0.078 | (Intercept) | 0.567 | 0.870 |
| 0.078 | BIM | 0.735 | 0.870 |
| 0.078 | CAVEOLIN1 | −0.040 | 0.870 |
| 0.078 | CYCLINB1 | 0.384 | 0.870 |
| 0.078 | NCADHERIN | 0.091 | 0.870 |
| 0.078 | PKCALPHA_pS657 | −0.293 | 0.870 |
| 0.078 | YAP_pS127 | −0.201 | 0.870 |
| 0.078 | PKCPANBETAII_pS660 | −0.017 | 0.870 |
| 0.078 | TIGAR | 0.103 | 0.870 |
| 0.078 | TFRC | 0.006 | 0.870 |
| 0.078 | ANNEXIN1 | −0.377 | 0.870 |
| 0.078 | DUSP4 | 0.025 | 0.870 |
| 0.078 | NRF2 | 0.851 | 0.870 |
| 0.078 | TTF1 | −0.175 | 0.870 |
| 0.088 | (Intercept) | 0.437 | 0.891 |
| 0.088 | BIM | 0.685 | 0.891 |
| 0.088 | CAVEOLIN1 | −0.040 | 0.891 |
| 0.088 | CYCLINB1 | 0.338 | 0.891 |
| 0.088 | NCADHERIN | 0.060 | 0.891 |
| 0.088 | PKCALPHA_pS657 | −0.257 | 0.891 |
| 0.088 | YAP_pS127 | −0.155 | 0.891 |
| 0.088 | PKCPANBETAII_pS660 | −0.040 | 0.891 |
| 0.088 | TIGAR | 0.023 | 0.891 |
| 0.088 | ANNEXIN1 | −0.328 | 0.891 |
| 0.088 | DUSP4 | 0.006 | 0.891 |
| 0.088 | NRF2 | 0.784 | 0.891 |
| 0.088 | TTF1 | −0.156 | 0.891 |
| 0.098 | (Intercept) | 0.288 | 0.870 |
| 0.098 | BIM | 0.650 | 0.870 |
| 0.098 | CAVEOLIN1 | −0.040 | 0.870 |
| 0.098 | CYCLINB1 | 0.288 | 0.870 |
| 0.098 | NCADHERIN | 0.036 | 0.870 |
| 0.098 | PKCALPHA_pS657 | −0.218 | 0.870 |
| 0.098 | YAP_pS127 | −0.112 | 0.870 |
| 0.098 | PKCPANBETAII_pS660 | −0.052 | 0.870 |
| 0.098 | ANNEXIN1 | −0.274 | 0.870 |
| 0.098 | NRF2 | 0.732 | 0.870 |
| 0.098 | TTF1 | −0.135 | 0.870 |
| 0.108 | (Intercept) | 0.134 | 0.870 |
| 0.108 | BIM | 0.623 | 0.870 |
| 0.108 | CAVEOLIN1 | −0.039 | 0.870 |
| 0.108 | CYCLINB1 | 0.239 | 0.870 |
| 0.108 | NCADHERIN | 0.017 | 0.870 |
| 0.108 | PKCALPHA_pS657 | −0.180 | 0.870 |
| 0.108 | YAP_pS127 | −0.071 | 0.870 |
| 0.108 | PKCPANBETAII_pS660 | −0.059 | 0.870 |
| 0.108 | ANNEXIN1 | −0.219 | 0.870 |
| 0.108 | NRF2 | 0.686 | 0.870 |
| 0.108 | TTF1 | −0.115 | 0.870 |
| 0.118 | (Intercept) | −0.015 | 0.826 |
| 0.118 | BIM | 0.600 | 0.826 |
| 0.118 | CAVEOLIN1 | −0.038 | 0.826 |
| 0.118 | CYCLINB1 | 0.193 | 0.826 |
| 0.118 | PKCALPHA_pS657 | −0.144 | 0.826 |
| 0.118 | YAP_pS127 | −0.032 | 0.826 |
| 0.118 | PKCPANBETAII_pS660 | −0.066 | 0.826 |
| 0.118 | ANNEXIN1 | −0.166 | 0.826 |
| 0.118 | NRF2 | 0.640 | 0.826 |
| 0.118 | TTF1 | −0.096 | 0.826 |
| 0.128 | (Intercept) | −0.164 | 0.826 |
| 0.128 | BIM | 0.584 | 0.826 |
| 0.128 | CAVEOLIN1 | −0.036 | 0.826 |
| 0.128 | CYCLINB1 | 0.137 | 0.826 |
| 0.128 | PKCALPHA_pS657 | −0.109 | 0.826 |
| 0.128 | FOXM1 | 0.030 | 0.826 |
| 0.128 | PKCPANBETAII_pS660 | −0.066 | 0.826 |
| 0.128 | ANNEXIN1 | −0.113 | 0.826 |
| 0.128 | NRF2 | 0.583 | 0.826 |
| 0.128 | TTF1 | −0.076 | 0.826 |
| 0.138 | (Intercept) | −0.305 | 0.783 |
| 0.138 | BIM | 0.569 | 0.783 |
| 0.138 | CAVEOLIN1 | −0.034 | 0.783 |
| 0.138 | CYCLINB1 | 0.072 | 0.783 |
| 0.138 | PKCALPHA_pS657 | −0.079 | 0.783 |
| 0.138 | FOXM1 | 0.066 | 0.783 |
| 0.138 | PKCPANBETAII_pS660 | −0.064 | 0.783 |
| 0.138 | ANNEXIN1 | −0.058 | 0.783 |
| 0.138 | NRF2 | 0.527 | 0.783 |
| 0.138 | TTF1 | −0.055 | 0.783 |
| 0.148 | (Intercept) | −0.444 | 0.761 |
| 0.148 | BIM | 0.556 | 0.761 |
| 0.148 | CAVEOLIN1 | −0.032 | 0.761 |
| 0.148 | CYCLINB1 | 0.009 | 0.761 |
| 0.148 | PKCALPHA_pS657 | −0.050 | 0.761 |
| 0.148 | FOXMI | 0.101 | 0.761 |
| 0.148 | PKCPANBETAII_pS660 | −0.063 | 0.761 |
| 0.148 | ANNEXIN1 | −0.004 | 0.761 |
| 0.148 | NRF2 | 0.474 | 0.761 |
| 0.148 | TTF1 | −0.034 | 0.761 |
| 0.158 | (Intercept) | −0.554 | 0.739 |
| 0.158 | BIM | 0.520 | 0.739 |
| 0.158 | CAVEOLIN1 | −0.022 | 0.739 |
| 0.158 | PKCALPHA_pS657 | −0.009 | 0.739 |
| 0.158 | FOXM1 | 0.074 | 0.739 |
| 0.158 | PKCPANBETAII_pS660 | −0.067 | 0.739 |
| 0.158 | NRF2 | 0.409 | 0.739 |
| 0.158 | TTF1 | −0.016 | 0.739 |
| 0.168 | (Intercept) | −0.656 | 0.739 |
| 0.168 | BIM | 0.477 | 0.739 |
| 0.168 | CAVEOLIN1 | −0.009 | 0.739 |
| 0.168 | FOXM1 | 0.036 | 0.739 |
| 0.168 | PKCPANBETAII_pS660 | −0.054 | 0.739 |
| 0.168 | NRF2 | 0.342 | 0.739 |
| 0.178 | (Intercept) | −0.693 | 0.717 |
| 0.178 | BIM | 0.432 | 0.717 |
| 0.178 | PKCPANBETAII_pS660 | −0.022 | 0.717 |
| 0.178 | NRF2 | 0.260 | 0.717 |
| 0.188 | (Intercept) | −0.699 | 0.717 |
| 0.188 | BIM | 0.368 | 0.717 |
| 0.188 | NRF2 | 0.152 | 0.717 |

TABLE 15
Subtype marker gene list. The table shows subtype marker
genes for each of the five LUAD expression subtypes.

| Subtype | Marker Genes |
|---|---|
| S1 | SCNN1D |
| S1 | MESP1 |
| S1 | ICAM5 |
| S1 | ARHGEF19 |
| S1 | DCST2 |
| S1 | ATP6V1C2 |
| S1 | C9orf173 |
| S1 | SPTBN5 |
| S1 | C19orf57 |
| S1 | LOC440040 |
| S1 | ITGA2B |
| S1 | SUSD4 |
| S1 | PNMT |
| S1 | PCP2 |
| S1 | CSPG5 |
| S1 | MESP2 |
| S1 | FBXL16 |
| S1 | CYP21A2 |
| S1 | TBX1 |
| S1 | DUSP5P |
| S1 | PAGE1 |
| S1 | ICAM4 |
| S1 | NR2E1 |
| S1 | PLXNB3 |
| S1 | UPK2 |
| S1 | TMEM88B |
| S1 | LOC84989 |
| S1 | LOC645323 |
| S1 | CLDN3 |
| S1 | FAM171A2 |
| S1 | SLC7A10 |
| S1 | KHDRBS2 |
| S1 | KLC3 |
| S1 | COL9A2 |
| S1 | NUP210L |
| S1 | LOC148709 |
| S1 | ZDHHC11 |
| S1 | LOC729668 |
| S1 | MLXIPL |
| S1 | CCDC114 |
| S1 | EFR3B |
| S1 | HSPB9 |
| S1 | SYCP2 |
| S1 | DLX3 |
| S1 | FBN3 |
| S1 | RTBDN |
| S1 | RGS11 |
| S1 | RNF222 |
| S1 | SRPK3 |
| S1 | RGMA |
| S1 | DMBX1 |
| S1 | WBSCR28 |
| S1 | SPINK2 |
| S1 | PLEKHG4B |
| S1 | ARX |
| S1 | KLRG2 |
| S1 | SLC16A9 |
| S1 | C20orf195 |
| S1 | HGFAC |
| S1 | OXT |
| S1 | PEG10 |
| S1 | GRHL3 |
| S1 | TMEM130 |
| S1 | CRYGN |
| S1 | LOC440356 |
| S1 | FZD9 |
| S1 | LOC100133669 |
| S1 | MUC21 |
| S1 | CYP1A1 |
| S1 | ALS2CR11 |
| S1 | ABCA17P |
| S1 | C2orf54 |
| S1 | WDR86 |
| S1 | EFHD1 |
| S1 | CLDN9 |
| S1 | COL28A1 |
| S1 | C1orf65 |
| S1 | CCDC37 |
| S1 | RYR1 |
| S1 | RNF126P1 |
| S1 | KRTAP3.1 |
| S1 | TEPP |
| S1 | USH1G |
| S1 | B3GNT7 |
| S1 | LRRN4 |
| S1 | DUSP9 |
| S1 | B4GALNT4 |
| S1 | AMOT |
| S1 | TCAM1P |
| S1 | FOXD3 |
| S1 | AMY2A |
| S1 | C14orf39 |
| S1 | SLC1A7 |
| S1 | APBA2 |
| S1 | SIX2 |
| S1 | UPK3A |
| S1 | ZNF560 |
| S1 | KISS1R |
| S1 | CACNG4 |
| S1 | COL11A2 |
| S2 | COL10A1 |
| S2 | ISM1 |
| S2 | SLC24A2 |
| S2 | THBS2 |
| S2 | ISLR |
| S2 | ITGA11 |
| S2 | OMD |
| S2 | FGF1 |
| S2 | LRRC17 |
| S2 | ST8SIA2 |
| S2 | TMEM90B |
| S2 | CORIN |
| S2 | COL8A2 |
| S2 | C7orf10 |
| S2 | KERA |
| S2 | MFAP5 |
| S2 | METTL11B |
| S2 | GPR88 |
| S2 | PDZRN4 |
| S2 | MATN3 |
| S2 | C1QTNF3 |
| S2 | FNDC1 |
| S2 | CYP26A1 |
| S2 | LRRC15 |
| S2 | ASPN |
| S2 | COL12A1 |
| S2 | MMP11 |
| S2 | RANBP3L |
| S2 | COL11A1 |
| S2 | CILP2 |
| S2 | PCDH19 |
| S2 | NKX3.2 |
| S2 | LOC283867 |
| S2 | CLSTN2 |
| S2 | NETO1 |
| S2 | CNIH3 |
| S2 | COL1A1 |
| S2 | CSMD2 |
| S2 | UBE2QL1 |
| S2 | MEGF10 |
| S2 | IBSP |
| S2 | STMN2 |
| S2 | SPOCK1 |
| S2 | KRT75 |
| S2 | SFRP2 |
| S2 | GPR1 |
| S2 | HAPLN1 |
| S2 | EDIL3 |
| S2 | C11orf41 |
| S2 | SPP1 |
| S2 | GRP |
| S2 | TNC |
| S2 | MMP8 |
| S2 | CST4 |
| S2 | PPAPDC1A |
| S2 | ITGB3 |
| S2 | CST1 |
| S2 | HEPHL1 |
| S2 | AK5 |
| S2 | NPTX2 |
| S2 | CILP |
| S2 | GAP43 |
| S2 | IGFL2 |
| S2 | COMP |
| S2 | CAPNS2 |
| S2 | CRHR1 |
| S2 | EPYC |
| S2 | ZPLD1 |
| S2 | SOX11 |
| S2 | GLYATL2 |
| S2 | ENPP3 |
| S2 | PRND |
| S2 | PPBP |
| S2 | PADI3 |
| S2 | MMP13 |
| S2 | KRT20 |
| S2 | SALL1 |
| S2 | PLAT |
| S2 | PODNL1 |
| S2 | LIPK |
| S2 | LECT1 |
| S2 | GRIN2A |
| S2 | MMP3 |
| S2 | SHISA3 |
| S2 | PADI1 |
| S2 | CD207 |
| S2 | CXorf64 |
| S2 | TCN1 |
| S2 | CCDC129 |
| S2 | CA10 |
| S2 | DIO1 |
| S2 | LRRTM1 |
| S2 | IGF2 |
| S2 | TGM5 |
| S2 | CXCL14 |
| S2 | PCDH8 |
| S2 | VTCN1 |
| S2 | DBC1 |
| S2 | TFAP2D |
| S2 | MYO3B |
| S3 | CD274 |
| S3 | GBP1 |
| S3 | CXCL10 |
| S3 | TBX21 |
| S3 | CXCL11 |
| S3 | AIM2 |
| S3 | CCL4 |
| S3 | PDCD1LG2 |
| S3 | FAM26F |
| S3 | OR4C6 |
| S3 | FBXL13 |
| S3 | PDCD1 |
| S3 | TGM4 |
| S3 | GBP5 |
| S3 | CD70 |
| S3 | KLRD1 |
| S3 | ARNTL2 |
| S3 | GZMB |
| S3 | C20orf141 |
| S3 | CXCL9 |
| S3 | CD8A |
| S3 | IFNG |
| S3 | NKG7 |
| S3 | GZMH |
| S3 | CCL5 |
| S3 | KLRC1 |
| S3 | FOSL1 |
| S3 | TNFRSF9 |
| S3 | ZNF683 |
| S3 | FASLG |
| S3 | KLRK1 |
| S3 | KLRC3 |
| S3 | GNLY |
| S3 | CLEC6A |
| S3 | CCL8 |
| S3 | MYH16 |
| S3 | RGS20 |
| S3 | DCBLD2 |
| S3 | KLRC2 |
| S3 | TTC24 |
| S3 | CXCR2P1 |
| S3 | C12orf70 |
| S3 | CSF2 |
| S3 | LHX1 |
| S3 | AFAP1L2 |
| S3 | ADAMDEC1 |
| S3 | LILRA3 |
| S3 | GPR84 |
| S3 | KCNJ10 |
| S3 | PLA2G2D |
| S3 | FBXO32 |
| S3 | MFI2 |
| S3 | CCBE1 |
| S3 | MYBL1 |
| S3 | LYPD5 |
| S3 | NIPAL4 |
| S3 | GOS2 |
| S3 | TGFBI |
| S3 | LOC100216001 |
| S3 | CDA |
| S3 | C10orf55 |
| S3 | AREG |
| S3 | LOC400696 |
| S3 | BATF3 |
| S3 | LICAM |
| S3 | EREG |
| S3 | PLAU |
| S3 | IL20RB |
| S3 | CD109 |
| S3 | C15orf48 |
| S3 | MET |
| S3 | GBP6 |
| S3 | TMEM156 |
| S3 | PMAIP1 |
| S3 | MT1A |
| S3 | SPRR2F |
| S3 | LOC554202 |
| S3 | LOC100126784 |
| S3 | BEND6 |
| S3 | XIRP1 |
| S3 | GJA3 |
| S3 | PDLIM4 |
| S3 | FGF5 |
| S3 | GPR115 |
| S3 | DUSP13 |
| S3 | PAPL |
| S3 | SBSN |
| S3 | CCL7 |
| S3 | FGFBP1 |
| S3 | CATSPER1 |
| S3 | TIMP4 |
| S3 | GREBIL |
| S3 | S100A2 |
| S3 | FHOD3 |
| S3 | KCNK12 |
| S3 | PNPLA5 |
| S3 | C9orf84 |
| S3 | HTR1D |
| S3 | TNNT1 |
| S3 | GPR87 |
| S4 | LOC441177 |
| S4 | LOC100190940 |
| S4 | KCNU1 |
| S4 | ZMAT4 |
| S4 | SLC38A8 |
| S4 | CPLX2 |
| S4 | CALCA |
| S4 | HOXD13 |
| S4 | PCSK1 |
| S4 | UGT3A1 |
| S4 | KLK14 |
| S4 | CGA |
| S4 | ASCL1 |
| S4 | STXBP5L |
| S4 | CALCB |
| S4 | HEPACAM2 |
| S4 | AGXT2L1 |
| S4 | RET |
| S4 | CPS1 |
| S4 | CALB1 |
| S4 | AKR1C4 |
| S4 | C6orf176 |
| S4 | PCK1 |
| S4 | GP2 |
| S4 | UGT2B4 |
| S4 | GLDC |
| S4 | FGA |
| S4 | F2 |
| S4 | FZD10 |
| S4 | NTS |
| S4 | MLLT11 |
| S4 | SCG3 |
| S4 | NEURL |
| S4 | ABCC2 |
| S4 | PAH |
| S4 | INHA |
| S4 | COL25A1 |
| S4 | DDC |
| S4 | FGL1 |
| S4 | INSL4 |
| S4 | NR0B1 |
| S4 | KLK13 |
| S4 | KLK12 |
| S4 | NKAIN2 |
| S4 | CYP4F3 |
| S4 | ZFP42 |
| S4 | MUC13 |
| S4 | CALML3 |
| S4 | HOXD11 |
| S4 | AKR1C2 |
| S4 | AKRIC1 |
| S4 | CHRNA9 |
| S4 | FGB |
| S4 | F7 |
| S4 | CTNND2 |
| S4 | FGG |
| S4 | EPS8L3 |
| S4 | CELF3 |
| S4 | SST |
| S4 | MAGEA4 |
| S4 | DLL3 |
| S4 | TFF1 |
| S4 | MSI1 |
| S4 | MAGEA1 |
| S4 | SLC6A15 |
| S4 | LIN28B |
| S4 | BARX1 |
| S4 | WDR72 |
| S4 | MAGEA9B |
| S4 | UCHL1 |
| S4 | GAL |
| S4 | GPX2 |
| S4 | TF |
| S4 | POPDC3 |
| S4 | CTAG2 |
| S4 | CSAG2 |
| S4 | C20orf70 |
| S4 | GNG4 |
| S4 | AKR1B10 |
| S4 | CTAG1B |
| S4 | C12orf39 |
| S4 | CSAG3 |
| S4 | CSAG1 |
| S4 | MAGEA2 |
| S4 | VIL1 |
| S4 | MAGEA12 |
| S4 | CRABP1 |
| S4 | PLUNC |
| S4 | KIF1A |
| S4 | HOXB8 |
| S4 | C12orf56 |
| S4 | KLK8 |
| S4 | MAGEA3 |
| S4 | MAGEA6 |
| S4 | PRAME |
| S4 | HOXB9 |
| S4 | CHGB |
| S4 | IGF2BP1 |
| S4 | GABRA3 |
| S4 | UGT1A6 |
| S5 | DUOX1 |
| S5 | CD300LG |
| S5 | GRIA1 |
| S5 | GKN2 |
| S5 | ADH1B |
| S5 | CACNA2D2 |
| S5 | SFTPD |
| S5 | CYP4B1 |
| S5 | EDN3 |
| S5 | PGC |
| S5 | INMT |
| S5 | DUOXA2 |
| S5 | CLDN18 |
| S5 | AFF3 |
| S5 | FIGF |
| S5 | TMEM132C |
| S5 | F11 |
| S5 | MACROD2 |
| S5 | AADAC |
| S5 | PLA2G1B |
| S5 | LOC723809 |
| S5 | PLA2G10 |
| S5 | PCDH15 |
| S5 | PEBP4 |
| S5 | ADH1A |
| S5 | ACADL |
| S5 | ABCA8 |
| S5 | SCGB3A2 |
| S5 | IHH |
| S5 | C16orf89 |
| S5 | WIFI |
| S5 | LGI3 |
| S5 | LRRK2 |
| S5 | CAPN9 |
| S5 | LOC149620 |
| S5 | RSPO2 |
| S5 | GDF10 |
| S5 | SFTPC |
| S5 | DPCR1 |
| S5 | ADAMTS8 |
| S5 | CA4 |
| S5 | SFTA1P |
| S5 | LRRC36 |
| S5 | VSIG2 |
| S5 | DUOX2 |
| S5 | AGER |
| S5 | SOSTDC1 |
| S5 | ASPG |
| S5 | CEACAM8 |
| S5 | ATP1A2 |
| S5 | CYP4Z2P |
| S5 | PRG4 |
| S5 | ODAM |
| S5 | SLC10A2 |
| S5 | RPL13AP17 |
| S5 | PCSK2 |
| S5 | IRX1 |
| S5 | HMGCS2 |
| S5 | C8orf34 |
| S5 | C20orf56 |
| S5 | ROBO2 |
| S5 | NXF3 |
| S5 | SLC26A9 |
| S5 | ZNF385B |
| S5 | SPATA18 |
| S5 | LOC150622 |
| S5 | SEC14L3 |
| S5 | SFTPA1 |
| S5 | HSD17B6 |
| S5 | FOLR1 |
| S5 | SLC6A4 |
| S5 | SFTPA2 |
| S5 | C2orf40 |
| S5 | CPB2 |
| S5 | LGALS4 |
| S5 | MEGF11 |
| S5 | CYP2B7P1 |
| S5 | ZBTB16 |
| S5 | LHFPL3 |
| S5 | CASR |
| S5 | NR0B2 |
| S5 | PCDH20 |
| S5 | ITLN2 |
| S5 | ERN2 |
| S5 | SPINK5 |
| S5 | KIAA0408 |
| S5 | CHIA |
| S5 | ANKFN1 |
| S5 | GJB1 |
| S5 | CLDN2 |
| S5 | DMBT1 |
| S5 | AZU1 |
| S5 | C6 |
| S5 | CAPN6 |
| S5 | SCTR |
| S5 | C13orf30 |
| S5 | TMEM132D |
| S5 | AQP5 |
| S5 | SCGB3A1 |
| S5 | PTPRT |

TABLE 16A
Results from S2 prediction models based on The Cancer Genome Atlas
(TCGA) lung adenocarcinoma (LUAD) gene expression data.

| Lambda | Predictors | Coefficients | Threshold | Accuracy |
|---|---|---|---|---|
| 0.009 | (Intercept) | −21.296 | 0.2 | 0.97 |
| | SLC24A2 | 0.164 | | |
| | C7orf10 | 0.104 | | |
| | MFAP5 | 0.175 | | |
| | GPR88 | 0.304 | | |
| | MATN3 | 0.098 | | |
| | FNDC1 | 0.277 | | |
| | RANBP3L | 0.066 | | |
| | CILP2 | 0.120 | | |
| | PCDH19 | 0.063 | | |
| | SPP1 | 0.216 | | |
| | CAPNS2 | 0.023 | | |
| | ZPLD1 | 0.085 | | |
| | ENPP3 | 0.167 | | |
| | PRND | 0.033 | | |
| | PLAT | 0.043 | | |
| | PODNL1 | 0.373 | | |
| | LIPK | 0.131 | | |
| | SHISA3 | 0.097 | | |
| | CXorf64 | 0.349 | | |
| | DIO1 | 0.070 | | |
| | PCDH8 | 0.137 | | |
| | DBC1 | 0.078 | | |
| | MYO3B | 0.078 | | |

TABLE 16B
Results from S2 prediction models based on The Cancer Genome Atlas
(TCGA) lung adenocarcinoma (LUAD) gene expression data.

| Lambda | Predictors | Coefficients | Threshold | Accuracy |
|---|---|---|---|---|
| 0.089 | (Intercept) | −3.968 | 0.1 | 0.960 |
| | SLC24A2 | 0.236 | | |
| | COL8A2 | 0.077 | | |
| | C7orf10 | 0.073 | | |
| | CYP26A1 | 0.078 | | |
| | MMP11 | 0.004 | | |

TABLE 17
Accuracy of S2 prediction models based on The Cancer Genome Atlas
(TCGA) lung adenocarcinoma (LUAD) gene expression data with
varying lambda values. The five-feature model is in bold. Each
model is preceded by the Intercept corresponding to the model.

| Lambda | Predictors | Coefficients | Accuracy |
|---|---|---|---|
| 0.009 | (Intercept) | −21.296 | 0.970 |
| 0.009 | SLC24A2 | 0.164 | 0.970 |
| 0.009 | C7orf10 | 0.104 | 0.970 |
| 0.009 | MFAP5 | 0.175 | 0.970 |
| 0.009 | GPR88 | 0.304 | 0.970 |
| 0.009 | MATN3 | 0.098 | 0.970 |
| 0.009 | FNDC1 | 0.277 | 0.970 |
| 0.009 | RANBP3L | 0.066 | 0.970 |
| 0.009 | CILP2 | 0.120 | 0.970 |
| 0.009 | PCDH19 | 0.063 | 0.970 |
| 0.009 | SPP1 | 0.216 | 0.970 |
| 0.009 | CAPNS2 | 0.023 | 0.970 |
| 0.009 | ZPLD1 | 0.085 | 0.970 |
| 0.009 | ENPP3 | 0.167 | 0.970 |
| 0.009 | PRND | 0.033 | 0.970 |
| 0.009 | PLAT | 0.043 | 0.970 |
| 0.009 | PODNL1 | 0.373 | 0.970 |
| 0.009 | LIPK | 0.131 | 0.970 |
| 0.009 | SHISA3 | 0.097 | 0.970 |
| 0.009 | CXorf64 | 0.349 | 0.970 |
| 0.009 | DIO1 | 0.070 | 0.970 |
| 0.009 | PCDH8 | 0.137 | 0.970 |
| 0.009 | DBC1 | 0.078 | 0.970 |
| 0.009 | MYO3B | 0.078 | 0.970 |
| 0.019 | (Intercept) | −13.861 | 0.970 |
| 0.019 | SLC24A2 | 0.208 | 0.970 |
| 0.019 | THBS2 | 0.048 | 0.970 |
| 0.019 | ITGA11 | 0.044 | 0.970 |
| 0.019 | C7orf10 | 0.115 | 0.970 |
| 0.019 | MFAP5 | 0.097 | 0.970 |
| 0.019 | GPR88 | 0.210 | 0.970 |
| 0.019 | MATN3 | 0.057 | 0.970 |
| 0.019 | FNDC1 | 0.084 | 0.970 |
| 0.019 | CYP26A1 | 0.024 | 0.970 |
| 0.019 | RANBP3L | 0.030 | 0.970 |
| 0.019 | CILP2 | 0.118 | 0.970 |
| 0.019 | PCDH19 | 0.039 | 0.970 |
| 0.019 | IBSP | 0.043 | 0.970 |
| 0.019 | SPP1 | 0.102 | 0.970 |
| 0.019 | ZPLD1 | 0.057 | 0.970 |
| 0.019 | ENPP3 | 0.106 | 0.970 |
| 0.019 | PODNL1 | 0.190 | 0.970 |
| 0.019 | LIPK | 0.084 | 0.970 |
| 0.019 | SHISA3 | 0.070 | 0.970 |
| 0.019 | CXorf64 | 0.269 | 0.970 |
| 0.019 | DIO1 | 0.025 | 0.970 |
| 0.019 | LRRTM1 | 0.001 | 0.970 |
| 0.019 | PCDH8 | 0.055 | 0.970 |
| 0.019 | DBC1 | 0.057 | 0.970 |
| 0.019 | MYO3B | 0.016 | 0.970 |
| 0.029 | (Intercept) | −10.093 | 0.970 |
| 0.029 | SLC24A2 | 0.219 | 0.970 |
| 0.029 | THBS2 | 0.092 | 0.970 |
| 0.029 | ITGA11 | 0.070 | 0.970 |
| 0.029 | ST8SIA2 | 0.013 | 0.970 |
| 0.029 | C7orf10 | 0.119 | 0.970 |
| 0.029 | MFAP5 | 0.023 | 0.970 |
| 0.029 | GPR88 | 0.169 | 0.970 |
| 0.029 | MATN3 | 0.013 | 0.970 |
| 0.029 | CYP26A1 | 0.089 | 0.970 |
| 0.029 | RANBP3L | 0.010 | 0.970 |
| 0.029 | CILP2 | 0.082 | 0.970 |
| 0.029 | IBSP | 0.080 | 0.970 |
| 0.029 | SPP1 | 0.029 | 0.970 |
| 0.029 | ZPLD1 | 0.034 | 0.970 |
| 0.029 | ENPP3 | 0.071 | 0.970 |
| 0.029 | PODNL1 | 0.103 | 0.970 |
| 0.029 | LIPK | 0.054 | 0.970 |
| 0.029 | SHISA3 | 0.048 | 0.970 |
| 0.029 | CXorf64 | 0.218 | 0.970 |
| 0.029 | PCDH8 | 0.014 | 0.970 |
| 0.029 | DBC1 | 0.042 | 0.970 |
| 0.039 | (Intercept) | −7.872 | 0.970 |
| 0.039 | SLC24A2 | 0.220 | 0.970 |
| 0.039 | THBS2 | 0.064 | 0.970 |
| 0.039 | ITGA11 | 0.079 | 0.970 |
| 0.039 | ST8SIA2 | 0.018 | 0.970 |
| 0.039 | COL8A2 | 0.002 | 0.970 |
| 0.039 | C7orf10 | 0.128 | 0.970 |
| 0.039 | GPR88 | 0.135 | 0.970 |
| 0.039 | CYP26A1 | 0.108 | 0.970 |
| 0.039 | CILP2 | 0.054 | 0.970 |
| 0.039 | IBSP | 0.084 | 0.970 |
| 0.039 | ZPLD1 | 0.010 | 0.970 |
| 0.039 | ENPP3 | 0.043 | 0.970 |
| 0.039 | PODNL1 | 0.046 | 0.970 |
| 0.039 | LIPK | 0.027 | 0.970 |
| 0.039 | SHISA3 | 0.026 | 0.970 |
| 0.039 | CXorf64 | 0.182 | 0.970 |
| 0.039 | DBC1 | 0.030 | 0.970 |
| 0.049 | (Intercept) | −6.632 | 0.960 |
| 0.049 | ISM1 | 0.008 | 0.960 |
| 0.049 | SLC24A2 | 0.217 | 0.960 |
| 0.049 | THBS2 | 0.028 | 0.960 |
| 0.049 | ITGA11 | 0.062 | 0.960 |
| 0.049 | ST8SIA2 | 0.016 | 0.960 |
| 0.049 | COL8A2 | 0.069 | 0.960 |
| 0.049 | C7orf10 | 0.129 | 0.960 |
| 0.049 | GPR88 | 0.097 | 0.960 |
| 0.049 | CYP26A1 | 0.107 | 0.960 |
| 0.049 | CILP2 | 0.027 | 0.960 |
| 0.049 | IBSP | 0.072 | 0.960 |
| 0.049 | ENPP3 | 0.017 | 0.960 |
| 0.049 | LIPK | 0.005 | 0.960 |
| 0.049 | SHISA3 | 0.002 | 0.960 |
| 0.049 | CXorf64 | 0.153 | 0.960 |
| 0.049 | DBC1 | 0.015 | 0.960 |
| 0.059 | (Intercept) | −5.745 | 0.960 |
| 0.059 | ISM1 | 0.006 | 0.960 |
| 0.059 | SLC24A2 | 0.224 | 0.960 |
| 0.059 | THBS2 | 0.009 | 0.960 |
| 0.059 | ITGA11 | 0.042 | 0.960 |
| 0.059 | ST8SIA2 | 0.009 | 0.960 |
| 0.059 | COL8A2 | 0.099 | 0.960 |
| 0.059 | C7orf10 | 0.124 | 0.960 |
| 0.059 | GPR88 | 0.066 | 0.960 |
| 0.059 | CYP26A1 | 0.106 | 0.960 |
| 0.059 | IBSP | 0.055 | 0.960 |
| 0.059 | CXorf64 | 0.114 | 0.960 |
| 0.069 | (Intercept) | −5.074 | 0.960 |
| 0.069 | SLC24A2 | 0.236 | 0.960 |
| 0.069 | ITGA11 | 0.021 | 0.960 |
| 0.069 | ST8SIA2 | 0.002 | 0.960 |
| 0.069 | COL8A2 | 0.102 | 0.960 |
| 0.069 | C7orf10 | 0.114 | 0.960 |
| 0.069 | GPR88 | 0.036 | 0.960 |
| 0.069 | CYP26A1 | 0.098 | 0.960 |
| 0.069 | MMP11 | 0.004 | 0.960 |
| 0.069 | IBSP | 0.033 | 0.960 |
| 0.069 | CXorf64 | 0.061 | 0.960 |
| 0.079 | (Intercept) | −4.549 | 0.960 |
| 0.079 | SLC24A2 | 0.242 | 0.960 |
| 0.079 | COL8A2 | 0.101 | 0.960 |
| 0.079 | C7orf10 | 0.101 | 0.960 |
| 0.079 | GPR88 | 0.006 | 0.960 |
| 0.079 | CYP26A1 | 0.088 | 0.960 |
| 0.079 | MMP11 | 0.010 | 0.960 |
| 0.079 | IBSP | 0.012 | 0.960 |
| 0.079 | CXorf64 | 0.008 | 0.960 |
| 0.089 | (Intercept) | −3.968 | 0.960 |
| 0.089 | SLC24A2 | 0.236 | 0.960 |
| 0.089 | COL8A2 | 0.077 | 0.960 |
| 0.089 | C7orf10 | 0.073 | 0.960 |
| 0.089 | CYP26A1 | 0.078 | 0.960 |
| 0.089 | MMP11 | 0.004 | 0.960 |
| 0.099 | (Intercept) | −3.407 | 0.960 |
| 0.099 | SLC24A2 | 0.221 | 0.960 |
| 0.099 | COL8A2 | 0.048 | 0.960 |
| 0.099 | C7orf10 | 0.041 | 0.960 |
| 0.099 | CYP26A1 | 0.064 | 0.960 |
| 0.109 | (Intercept) | −2.896 | 0.960 |
| 0.109 | SLC24A2 | 0.202 | 0.960 |
| 0.109 | COL8A2 | 0.020 | 0.960 |
| 0.109 | C7orf10 | 0.009 | 0.960 |
| 0.109 | CYP26A1 | 0.048 | 0.960 |
| 0.119 | (Intercept) | −2.545 | 0.960 |
| 0.119 | SLC24A2 | 0.169 | 0.960 |
| 0.119 | CYP26A1 | 0.024 | 0.960 |

TABLE 18A
Results from S2 prediction models based on The Cancer Genome
Atlas (TCGA) lung adenocarcinoma (LUAD) reverse-phase
protein array (RPPA) data.

| Lambda | Predictors | Coefficients | Threshold | Accuracy |
|---|---|---|---|---|
| 0.031 | (Intercept) | −2.977 | 0.1 | 0.891 |
| | BIM | −0.646 | | |
| | CMET_pY1235 | −0.179 | | |
| | CASPASE7CLEAVEDD198 | −0.349 | | |
| | CLAUDIN7 | −0.303 | | |
| | CYCLINE1 | −0.178 | | |
| | EGFR_pY1068 | 0.096 | | |
| | FIBRONECTIN | 0.088 | | |
| | INPP4B | 0.110 | | |
| | MAPK_pT202Y204 | 0.106 | | |
| | P27 | −0.751 | | |
| | PAXILLIN | 0.025 | | |
| | PCNA | −0.281 | | |
| | SMAD4 | −0.060 | | |
| | ARAF_pS299 | 1.059 | | |
| | BAP1C4 | −0.405 | | |
| | MYOSINIIA_pS1943 | 0.317 | | |
| | P21 | 0.716 | | |
| | SHP2_pY542 | 0.073 | | |
| | P63 | 0.014 | | |

TABLE 18B
Results from S2 prediction models based on The Cancer
Genome Atlas (TCGA) lung adenocarcinoma (LUAD)
reverse-phase protein array (RPPA) data.

| Lambda | Predictors | Coefficients | Threshold | Accuracy |
|---|---|---|---|---|
| 0.066 | (Intercept) | −2.279 | 0.1 | 0.89 |
| | BIM | −0.547 | | |
| | CLAUDIN7 | −0.010 | | |
| | EGFR_pY1068 | 0.019 | | |
| | P27 | −0.139 | | |
| | ARAF_pS299 | 0.477 | | |

TABLE 19
Accuracy of S2 prediction models based on The Cancer Genome
Atlas (TCGA) lung adenocarcinoma (LUAD) reverse-phase
protein array (RPPA) data with varying lambda values. The
five-feature model is in bold. Each model is preceded by
the Intercept corresponding to the model.

| Lambda | Predictors | Coefficients | Accuracy |
|---|---|---|---|
| 0.0312 | (Intercept) | −2.977 | 0.891 |
| 0.0312 | BIM | −0.646 | 0.891 |
| 0.0312 | CMET_pY1235 | −0.179 | 0.891 |
| 0.0312 | CASPASE7CLEAVEDD198 | −0.349 | 0.891 |
| 0.0312 | CLAUDIN7 | −0.303 | 0.891 |
| 0.0312 | CYCLINE1 | −0.178 | 0.891 |
| 0.0312 | EGFR_pY1068 | 0.096 | 0.891 |
| 0.0312 | FIBRONECTIN | 0.088 | 0.891 |
| 0.0312 | INPP4B | 0.110 | 0.891 |
| 0.0312 | MAPK_pT202Y204 | 0.106 | 0.891 |
| 0.0312 | P27 | −0.751 | 0.891 |
| 0.0312 | PAXILLIN | 0.025 | 0.891 |
| 0.0312 | PCNA | −0.281 | 0.891 |
| 0.0312 | SMAD4 | −0.060 | 0.891 |
| 0.0312 | ARAF_pS299 | 1.059 | 0.891 |
| 0.0312 | BAP1C4 | −0.405 | 0.891 |
| 0.0312 | MYOSINIIA_pS1943 | 0.317 | 0.891 |
| 0.0312 | P21 | 0.716 | 0.891 |
| 0.0312 | SHP2_pY542 | 0.073 | 0.891 |
| 0.0312 | P63 | 0.014 | 0.891 |
| 0.0362 | (Intercept) | −2.756 | 0.891 |
| 0.0362 | BIM | −0.725 | 0.891 |
| 0.0362 | CMET_pY1235 | −0.066 | 0.891 |
| 0.0362 | CASPASE7CLEAVEDD198 | −0.288 | 0.891 |
| 0.0362 | CLAUDIN7 | −0.260 | 0.891 |
| 0.0362 | CYCLINE1 | −0.034 | 0.891 |
| 0.0362 | EGFR_pY1068 | 0.089 | 0.891 |
| 0.0362 | FIBRONECTIN | 0.054 | 0.891 |
| 0.0362 | INPP4B | 0.105 | 0.891 |
| 0.0362 | MAPK_pT202Y204 | 0.058 | 0.891 |
| 0.0362 | P27 | −0.597 | 0.891 |
| 0.0362 | PCNA | −0.276 | 0.891 |
| 0.0362 | ARAF_pS299 | 0.983 | 0.891 |
| 0.0362 | BAP1C4 | −0.243 | 0.891 |
| 0.0362 | MYOSINIIA_pS1943 | 0.245 | 0.891 |
| 0.0362 | P21 | 0.488 | 0.891 |
| 0.0362 | SHP2_pY542 | 0.026 | 0.891 |
| 0.0412 | (Intercept) | −2.602 | 0.891 |
| 0.0412 | BIM | −0.749 | 0.891 |
| 0.0412 | CASPASE7CLEAVEDD198 | −0.228 | 0.891 |
| 0.0412 | CLAUDIN7 | −0.226 | 0.891 |
| 0.0412 | EGFR_pY1068 | 0.082 | 0.891 |
| 0.0412 | FIBRONECTIN | 0.034 | 0.891 |
| 0.0412 | INPP4B | 0.097 | 0.891 |
| 0.0412 | MAPK_pT202Y204 | 0.005 | 0.891 |
| 0.0412 | P27 | −0.496 | 0.891 |
| 0.0412 | PCNA | −0.236 | 0.891 |
| 0.0412 | ARAF_pS299 | 0.918 | 0.891 |
| 0.0412 | BAP1C4 | −0.098 | 0.891 |
| 0.0412 | MYOSINIIA_pS1943 | 0.150 | 0.891 |
| 0.0412 | P21 | 0.282 | 0.891 |
| 0.0462 | (Intercept) | −2.475 | 0.891 |
| 0.0462 | BIM | −0.753 | 0.891 |
| 0.0462 | CASPASE7CLEAVEDD198 | −0.160 | 0.891 |
| 0.0462 | CLAUDIN7 | −0.195 | 0.891 |
| 0.0462 | EGFR_pY1068 | 0.066 | 0.891 |
| 0.0462 | FIBRONECTIN | 0.044 | 0.891 |
| 0.0462 | INPP4B | 0.086 | 0.891 |
| 0.0462 | P27 | −0.446 | 0.891 |
| 0.0462 | PCNA | −0.120 | 0.891 |
| 0.0462 | ARAF_pS299 | 0.850 | 0.891 |
| 0.0462 | MYOSINIIA_pS1943 | 0.064 | 0.891 |
| 0.0462 | P21 | 0.021 | 0.891 |
| 0.0512 | (Intercept) | −2.395 | 0.891 |
| 0.0512 | BIM | −0.725 | 0.891 |
| 0.0512 | CASPASE7CLEAVEDD198 | −0.100 | 0.891 |
| 0.0512 | CLAUDIN7 | −0.152 | 0.891 |
| 0.0512 | EGFR_pY1068 | 0.057 | 0.891 |
| 0.0512 | INPP4B | 0.071 | 0.891 |
| 0.0512 | P27 | −0.370 | 0.891 |
| 0.0512 | PCNA | −0.018 | 0.891 |
| 0.0512 | ARAF_pS299 | 0.776 | 0.891 |
| 0.0512 | MYOSINIIA_pS1943 | 0.011 | 0.891 |
| 0.0562 | (Intercept) | −2.353 | 0.891 |
| 0.0562 | BIM | −0.683 | 0.891 |
| 0.0562 | CASPASE7CLEAVEDD198 | −0.041 | 0.891 |
| 0.0562 | CLAUDIN7 | −0.101 | 0.891 |
| 0.0562 | EGFR_pY1068 | 0.047 | 0.891 |
| 0.0562 | INPP4B | 0.045 | 0.891 |
| 0.0562 | P27 | −0.286 | 0.891 |
| 0.0562 | ARAF_pS299 | 0.688 | 0.891 |
| 0.0612 | (Intercept) | −2.315 | 0.891 |
| 0.0612 | BIM | −0.626 | 0.891 |
| 0.0612 | CLAUDIN7 | −0.053 | 0.891 |
| 0.0612 | EGFR_pY1068 | 0.035 | 0.891 |
| 0.0612 | INPP4B | 0.017 | 0.891 |
| 0.0612 | P27 | −0.212 | 0.891 |
| 0.0612 | ARAF_pS299 | 0.593 | 0.891 |
| 0.0662 | (Intercept) | −2.279 | 0.891 |
| 0.0662 | BIM | −0.547 | 0.891 |
| 0.0662 | CLAUDIN7 | −0.010 | 0.891 |
| 0.0662 | EGFR_pY1068 | 0.019 | 0.891 |
| 0.0662 | P27 | −0.139 | 0.891 |
| 0.0662 | ARAF_pS299 | 0.477 | 0.891 |
| 0.0712 | (Intercept) | −2.243 | 0.891 |
| 0.0712 | BIM | −0.467 | 0.891 |
| 0.0712 | P27 | −0.034 | 0.891 |
| 0.0712 | ARAF_pS299 | 0.342 | 0.891 |
| 0.0762 | (Intercept) | −2.212 | 0.891 |
| 0.0762 | BIM | −0.352 | 0.891 |
| 0.0762 | ARAF_pS299 | 0.191 | 0.891 |

Over the past several years, lung cancer subtypes have been studied to reveal new biology associated with clinical outcomes. The subtypes identified in these studies were consistent with the PI, PP, and TRU subtypes defined in the original The Cancer Genome Atlas (TCGA) study, and the subtypes defined herein from integrating genomic and proteomic data also align well with these original subtypes. Importantly, the analyses presented herein were sufficiently powered to further partition the PI subtype into 3 further subgroups as well as further characterize the biological features of the S1-S5 subtypes.

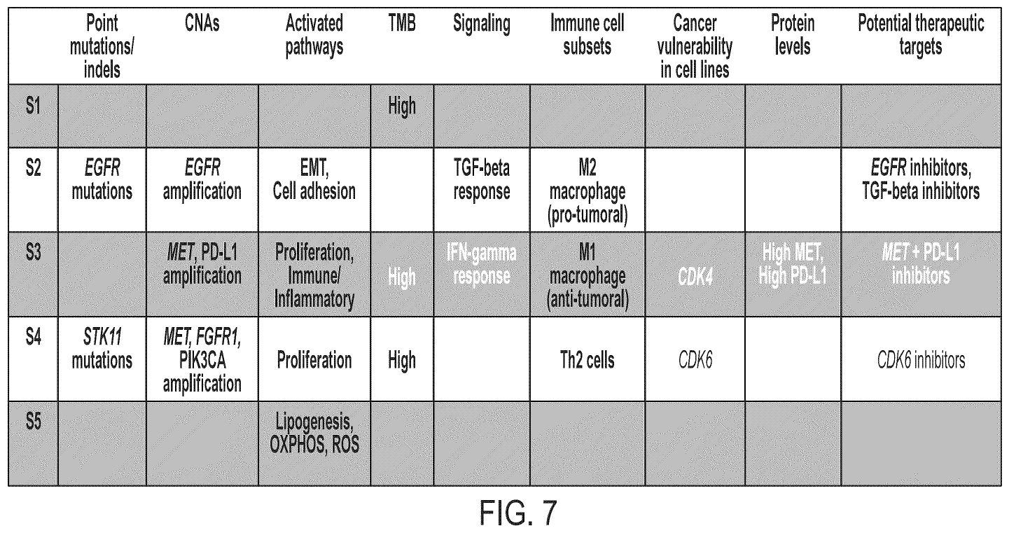

Multiple subtype-specific significantly recurrent mutations (point mutations, indels, and SCNAs) were identified that the previous The Cancer Genome Atlas (TCGA) lung adenocarcinoma (LUAD) study (Cancer Genome Atlas Research Network. “Comprehensive molecular profiling of lung adenocarcinoma.” Nature 511.7511 (2014): 543-550) did not detect, and was likely underpowered to detect (FIG. 7). These findings have shown that the newly identified expression subtypes were associated with distinct tumor biology and can serve as biomarkers of response to targeted therapies (e.g., response to EGFR inhibitors and TGF-beta inhibitors for S2; response to PD-L1, MET, and CDK4 inhibitors for S3; and resistance to PD-1 inhibitors for S4 due to STK11 mutations (FIG. 7)). Integrative analysis of The Cancer Genome Atlas (TCGA) and DepMap data also demonstrated the proof-of-concept idea that leveraging the genome-wide CRISPR screening data and expression subtypes of cell lines can identify novel therapeutic targets for expression subtypes.

The proteogenomic analysis presented herein provided additional support for the need to take into account not only copy-number alterations but also mRNA and protein expression when characterizing the biology of the different subtypes. For example, while not intending to be bound by theory, the observation that MET amplification had a profound impact on its protein expression in S3 but not in the other subtypes suggested that the mRNA and protein expression of these genes may, in some cases, be affected by a negative feedback loop or other types of regulation that reduces the effect of the increased DNA copy-number. Collectively, these results highlighted the importance of integrating the analysis between genomic and proteomic data to reveal underlying subtype-specific biology.

Analysis of proteogenomic data suggested that MET amplification in S3 tumors can lead to cell proliferation through the GAB1/AKT1 axis. S3 tumor-specific positive correlation between MET gene expression and PD-L1 gene expression also demonstrated that the MET gene may regulate PD-L1 expression in S3 tumors through GSK3β. A recent study demonstrated that MET amplification attenuates immunotherapy response by inhibiting STING in lung cancer and that targeted MET inhibition could increase the efficacy of immunotherapy. In the data presented herein, the MET-STING axis was only in S4, but not in S3, suggesting that the MET-GSK3β-PD-L1 axis may play a more important role in S3 than the MET-STING axis. Thus, while not intending to be bound by theory, in S3, MET might be a core regulator of two important cancer-related functions: (i) immune escape by upregulating PD-L1 expression, and (ii) proliferation through a synergistic effect with increased expression of BCL2L1 and MCM-family members (FIG. 15B). As increased PD-L1 expression is associated with suppression of anti-tumor immunity, these results might serve as evidence to explain one reason why the c-MET inhibitor tivantinib has performed poorly in various cancer clinical trials. Consequently, combination therapy targeting MET and PD-L1 could be synergistic for S3 tumors. Additionally, since S3 tumors also have relatively high TMB and interferon-gamma gene expression signature, this tumor subtype that accounts for approximately 20% of all lung adenocarcinoma (LUAD) patients (105 out of 509 The Cancer Genome Atlas (TCGA) lung adenocarcinoma (LUAD) tumors) likely responds well to combined MET inhibitors and PD-L1 blockade.

Since S3 cell lines also had CDK4 cancer vulnerability and showed high response to CDK4 inhibitors (FIG. 12C), S3 tumors likely also respond well to combined CDK4 inhibitors and PD-L1 blockade. Overall, the findings raise a clinical therapeutic hypothesis that membership in the S3 subtype can serve as a biomarker of response to combination immunotherapy targeting CDK4 or MET together with PD-L1 inhibitors. Since S4 tumors showed recurrent CCND3 (the CDK6-cyclin D3 complex gene) amplification in The Cancer Genome Atlas (TCGA) analysis, and S4-associated cell lines showed S4-specific cancer vulnerability in CDK6 (FIG. 2B) in the CRISPR KO analysis, specifically targeted CDK6 inhibitors can likely be used for treatment of S4 tumors.

Overall, the experiments provided herein demonstrate that a BayesianNMF approach can identify novel tumor expression subtypes, and that integrative analysis of multi-modal data (genomics, proteomics, and CRISPR screening data) can identify subtype-specific cancer vulnerabilities and subtype-specific biology. Moreover, since expression subtypes can represent both the tumor cells and their microenvironment—both of which can contribute to treatment response or resistance—expression subtype-centric integration of multi-modal data can identify more clinically relevant tumor subtypes. Other types of multi-modal data, such as single-cell RNA sequencing data, can allow single-cell level characterization of tumor expression subtypes, which can potentially reveal new subtype-specific biology as well as cell types and states associated with clinical outcomes.

In summary, integrative analysis of genomic, proteomic, and drug dependency data, robust lung adenocarcinoma expression subtypes were identified and subtype-specific biomarkers of response were found, including to CDK4/6, MET, and PD-L1 inhibitors. Lung adenocarcinoma (LUAD) is one of the most common cancer types with various available treatment modalities. However, better biomarkers of response are still needed for further improving precision medicine. Therefore, a robust LUAD subtyping can substantially aid in determining the most effective therapies that target subtype-specific vulnerabilities. In the examples provided herein, multiple datasets were integrated: (i) the full 509 LUAD patient cohort from The Cancer Genome Atlas (TCGA) project, (ii) cancer vulnerability data in LUAD cell lines from the Broad Institute's DependencyMap, and (iii) proteomic data from the Clinical Proteomic Tumor Analysis Consortium (CPTAC) LUAD patients. Using these datasets, 5 expression subtypes (S1-S5) were identified with unique proteogenomic and dependency profiles that increased the resolution of previously defined subtypes (Proximal Inflammatory [PI]; Proximal Proliferative [PP]; and Terminal Respiratory Unit [TRU]). S4-associated cell lines exhibited specific vulnerability to CDK6 and CDK6-cyclin D3 complex gene, CCND3. S3 was characterized by dependency on CDK4, immune-related expression patterns, and altered MET signaling. Experimental validation showed that S3-associated cell lines responded to MET inhibitors, which also led to increased PD-L1 expression. Finally, a small set of biomarkers was identified for S3 and S4 that can be used in the clinic to classify patients into our therapeutically relevant subtypes. Overall, the lung adenocarcinoma expression subtypes, especially S3 that represents 20% of LUAD patients and S4 that represents 25% of LUAD patients, and their biomarkers can help identify patients likely to respond to CDK4/6, MET, or PD-L1 inhibitors, improving patient outcome.

The results described above were obtained using the following methods and materials.

### The Cancer Genome Atlas (TCGA) Lung Adenocarcinoma (LUAD) Expression Matrix

Batch-corrected upper quartile normalized RSEM (RNA-Seq by Expectation-Maximization) data for The Cancer Genome Atlas (TCGA) lung adenocarcinoma (LUAD) cohort from the PanCanAtlas study (Hoadley, Katherine A., et al. “Cell-of-origin patterns dominate the molecular classification of 10,000 tumors from 33 types of cancer.” Cell 173.2 (2018): 291-304) was used for analysis.

### Identification of the Cancer Genome Atlas (TCGA) Lung Adenocarcinoma (LUAD) Expression Subtypes and Subtype Labeling in Cancer Cell Line Encyclopedia (CCLE) and Clinical Proteomic Tumor Analysis Consortium (CPTAC) LUAD Samples

For expression subtyping, BayesNMF (Tan, Vincent Y F, and Cédric Févotte. “Automatic relevance determination in nonnegative matrix factorization with the/spl beta/-divergence.” IEEE Transactions on Pattern Analysis and Machine Intelligence 35.7 (2012): 1592-1605) with a consensus hierarchical clustering approach was applied to the log₂(RSEM) The Cancer Genome Atlas (TCGA) lung adenocarcinoma (LUAD) gene expression data as described in Robertson, A. Gordon, et al. “Comprehensive molecular characterization of muscle-invasive bladder cancer.” Cell 171.3 (2017): 540-556, Kim, Jaegil, et al. “The cancer genome atlas expression subtypes stratify response to checkpoint inhibition in advanced urothelial cancer and identify a subset of patients with high survival probability.” European urology 75.6 (2019): 961-964, and Taylor-Weiner, Amaro, et al. “Scaling computational genomics to millions of individuals with GPUs.” Genome biology 20.1 (2019): 1-5. Expression subtype classifiers were then derived as described in Kim, Jaegil, et al. “The cancer genome atlas expression subtypes stratify response to checkpoint inhibition in advanced urothelial cancer and identify a subset of patients with high survival probability.” European urology 75.6 (2019): 961-964. Using differentially over-expressed subtype markers (100 marker genes in each subtype) in TCAG lung adenocarcinoma (LUAD) expression subtypes, an association of the new sample from the Cancer Cell Line Encyclopedia (CCLE) and CPTAC RNA-seq samples to The Cancer Genome Atlas (TCGA) lung adenocarcinoma (LUAD) expression subtypes was determined. Cancer Cell Line Encyclopedia (CCLE) and CPTAC RNA-seq samples were assigned to one of the five identified The Cancer Genome Atlas (TCGA) lung adenocarcinoma (LUAD) expression subtypes if the normalized association with one of The Cancer Genome Atlas (TCGA) subtypes was larger than 0.6.

### Mutation Significance Analysis

MutSig2CV (Lawrence, Michael S., et al. “Mutational heterogeneity in cancer and the search for new cancer-associated genes.” Nature 499.7457 (2013): 214-218, Lawrence, Michael S., et al. “Discovery and saturation analysis of cancer genes across 21 tumor types.” Nature 505.7484 (2014): 495-501) was applied to identify significantly mutated genes and GISTIC 2.0 (Mermel, Craig H., et al. “GISTIC2. 0 facilitates sensitive and confident localization of the targets of focal somatic copy-number alteration in human cancers.” Genome biology 12.4 (2011): 1-14) was applied to identify significant focal copy number alterations in a cohort of samples of interest (all The Cancer Genome Atlas (TCGA) lung adenocarcinoma (LUAD) samples, each of five TCGA lung adenocarcinoma (LUAD) expression subtypes). Due to small sample size of Clinical Proteomic Tumor Analysis Consortium (CPTAC) lung adenocarcinoma (LUAD) cohort (n=1 for S1, n=2 for S2, n=13 for S3, and n=13 for S4), MutSig2CV and GISTIC 2.0 could not be applied for CPTAC lung adenocarcinoma (LUAD) cohort. As an alternative, the proportion of samples with recurrent somatic copy-number alterations (SCNAs) in the TCGA lung adenocarcinoma (LUAD) cohort with those in the CPTAC lung adenocarcinoma (LUAD) cohort was compared.

### Pathway Analysis

Single-sample gene set variance analysis (GSVA) was performed using the gsva function (method=“gsva”, mx.diff=TRUE) from the R package ‘GSVA’ (v.1.30.0). GSVA implements a non-parametric method of gene set enrichment to generate an enrichment score for each gene set within a sample. The Molecular Signatures Database (MSigDB) gene sets v.6.1 were used to represent broad biological processes. The pathways with significantly different activities across the subtypes were identified based on FDR-adjusted P value <0.05 and mean difference of GSVA enrichment scores between subtypes of interest vs. others >0.2 or <−0.2.

### Survival Analysis

Disease-specific survival information of The Cancer Genome Atlas (TCGA) lung adenocarcinoma (LUAD) patients (‘DSS’: disease-specific survival event, ‘DSS.time’: disease-specific survival time) and other clinicopathologic variables were obtained from an integrated TCGA pan-cancer clinical data resource. Kaplan-Meier curves (with the log-rank test P values) were plotted using the Surv function in the R package ‘survival’ (v.2.43-1).

### Biomarker Analysis

Biomarker discovery was done by applying lasso logistic regression on either gene expression data or reverse-phase protein array (RPPA) data (level 4 RPPA data were obtained from the Cancer Proteome Atlas Portal) from The Cancer Genome Atlas (TCGA) lung adenocarcinoma (LUAD) cohort (randomly split into 80% training data and 20% test data) to predict subtypes of interest (S3 vs. others or S4 vs. others). For gene expression data, 100 subtype marker genes were used (Table 15) as the potential features to test. The lambda value was chosen to minimize the prediction error rate using the cv.glmnet( ) function in the R package ‘glmnet’ (v.4.1-1). Threshold values from 0.1 to 1 in increments of 0.1 were tested for the threshold selection that maximizes AUC values. Accuracy of the model was based on the agreement of the predicted subtypes and the true subtype label in the test data. To reduce the number of features down to five for the 5-feature models, the model was forced to reduce the number of features down to five by increasing the lambda value which controls the amount of the coefficient shrinkage.

### Public Datasets

The Cancer Genome Atlas (TCGA) lung adenocarcinoma (LUAD) expression matrix was obtained from the PanCanAtlas study (Hoadley, Katherine A., et al. “Cell-of-origin patterns dominate the molecular classification of 10,000 tumors from 33 types of cancer.” Cell 173.2 (2018): 291-304). Survival data for TCGA lung adenocarcinoma (LUAD) samples was obtained from the integrated TCGA pan-cancer clinical data (Liu, Jianfang, et al. “An integrated TCGA pan-cancer clinical data resource to drive high-quality survival outcome analytics.” Cell 173.2 (2018): 400-416). The omics data and CRISPR knockout data for Cancer Cell Line Encyclopedia (CCLE) lung adenocarcinoma (LUAD) cell line samples were obtained from the Dependency Map (DepMap) portal (depmap.org/portal/; DepMap Public 21Q2 dataset) (Tsherniak, Aviad, et al. “Defining a cancer dependency map.” Cell 170.3 (2017): 564-576). Genomics and proteomics data for Clinical Proteomic Tumor Analysis Consortium (CPTAC) lung adenocarcinoma (LUAD) samples were obtained from Gillette, Michael A., et al. “Proteogenomic characterization reveals therapeutic vulnerabilities in lung adenocarcinoma.” Cell 182.1 (2020): 200-225. Proteomics datasets were obtained from though the Clinical Proteomic Tumor Analysis Consortium (CPTAC) data portal lung adenocarcinoma (LUAD):cptac-data-portal.georgetown.edu/study-summary/S054.

Proteomics data processing was done as described in Gillette, Michael A., et al. “Proteogenomic characterization reveals therapeutic vulnerabilities in lung adenocarcinoma.” Cell 182.1 (2020): 200-225.

### Statistical Analysis

Statistical analysis was performed using R. Statistical tests included a two-sided Wilcoxon rank-sum test, and Chi-squared test.

### Antibodies and Reagents

The following antibody was used for immunofluorescence staining—Recombinant Alexa Fluor® 488 Anti-PD-L1 antibody (ab209959). DAPI for nuclear staining (10236276001; Sigma-Aldrich), C-Met inhibitor Tivantinib was purchased from Selleck Chemicals (Houston, TX, USA), CDK4/6 inhibitor Palbociclib (PD 0332991 isethionate; Sigma-Aldrich), CDK4/6 Inhibitor IV (CAS 359886-84-3—Calbiochem).

### Cell Cultures

lung adenocarcinoma (LUAD) cell lines were used (HCC78, HCC827, NCIH1975, NCIH1838, NCIH1395, NCIH1833, NCIH1755, ABC1, CALU3). Tests for mycoplasma contamination were negative. Cells were maintained in RPMI 1640 medium supplemented with 10% fetal bovine serum and 1% penicillin-streptomycin.

### Proliferation Assay

Cells were seeded in duplicates (1×10⁴ in 96 well plates) and treated with DMSO, Tivantinib (3 μM) or a CDK4/6 inhibitor (Palbociclib-CDK4 concentration—11 nM/CDK4/6 concentration—16 nM; and CDK4/6 Inhibitor IV-CDK4 specific concentration—1.5 μM). The media and drugs were replenished every 2-3 days. Continuous cell growth was monitored in 96-well plates every 3 hr for 4 days using the IncuCyte Kinetic Imaging System. The relative confluency was analyzed using IncuCyte software. The reported response percentage for each cell line was calculated as the percent of confluency compared to their DMSO treated counterpart. Proliferation assays were repeated 4 times.

### Immunofluorescence Microscopy

Cells were seeded in duplicates (5×10⁴ in 24 well plates) and grown for 2-3 days and treated with DMSO or tivantinib. Cells were then fixed in 4% paraformaldehyde for 10 min and washed twice in cold PBS. Fluor® 488 Anti-PD-L1 antibody was added for 1 hr incubation in a light protected environment at room temperature followed by staining nuclei with DAPI. Fluorescence images were captured using Invitrogen™ EVOS™ FL Imaging System by Thermo Fisher Scientific. The fluorescent increase was further quantified using ImageJ software.

### Other Embodiments

From the foregoing description, it will be apparent that variations and modifications may be made to the disclosure provided herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

LENGTHY TABLES
The patent application contains a lengthy table section. A copy of the table is available in electronic form from the USPTO web site (https://seqdata.uspto.gov/?pageRequest=docDetail&DocID=US20240336981A1). An electronic copy of the table will also be available from the USPTO upon request and payment of the fee set forth in 37 CFR 1.19(b)(3).

1-137. (canceled)

138. A panel selected from the following:
a) a panel for characterizing an S3 subtype lung adenocarcinoma in a biological sample of a subject, the panel comprising two or more polypeptide or polynucleotide markers, or fragments thereof, selected from the following groups consisting of:
AIM2, CD274, DCBLD2, FBXO32, and MYBL1;
AFAP1L2, AIM2, ANNEXINVII, ARNTL2, BATF3, BRD4, C10orf55, C12orf70, C15orf48, CATSPER1, CD20, CD274, CD70, CD8A, CDA, CHK1_pS345, CSF2, DCBLD2, DJ1, ERALPHA, FBXO32, GATA3, GATA6, GBP1, GPR84, GZMB, IFNG, JAK2, KCNK12, LCK, MET, MIG6, MYBL1, NKG7, P63, P70S6K1, PDCD1, PDL1, PEA15, PI3KP110ALPHA, PKCDELTA_pS664, S100A2, SYNAPTOPHYSIN, TBX21, TGM4, TIGAR, TMEM156, and TTF1;
AFAP1L2, ARNTL2, BATF3, C15orf48, CATSPER1, CD274, CD70, CD8A, CDA, CSF2, DCBLD2, FBXO32, GPR84, GZMB, KCNK12, MET, MYBL1, NKG7, S100A2, TBX21, TGM4, and TMEM156;
ANNEXINVII, BRD4, CD20, CHK1_pS345, DJ1, ERALPHA, GATA3, GATA6, JAK2, LCK, MIG6, P63, PDCD1, PDL1, PEA15, PI3KP110ALPHA, PKCDELTA_pS664, SYNAPTOPHYSIN, TIGAR, and TTF1; and
GATA6, JAK2, MIG6, P70S6K1, and PDL1;
b) a panel for characterizing an S4 subtype lung adenocarcinoma in a biological sample of a subject, the panel comprising two or more polypeptide or polynucleotide markers, or fragments thereof, selected from the groups consisting of:
AKR1C4, CALCA, HOXD13, MLLT11, and PAH;
ACETYLATUBULINLYS40, AKR1C2, AKR1C4, AMPKALPHA, ANNEXIN1, BIM, C12orf39, C12orf56, C20orf70, CALB1, CALCA, CASPASE7CLEAVEDD198, CAVEOLIN1, CPS1, CSAG2, CYCLINB1, DUSP4, F2, F7, FOXM1, GLDC, GNG4, HEPACAM2, HOXD11, HOXD13, IGF2BP1, INSL4, JNK2, KCNU1, KLK14, LOC100190940, LOC441177, MIG6, MLLT11, MSH6, MTOR_pS2448, NAPSINA, NCADHERIN, NRF2, P38MAPK, P90RSK, PAH, PCSK1, PEA15, PKCALPHA_pS657, PKCPANBETAII_pS660, POPDC3, SLC38A8, SYNAPTOPHYSIN, TFRC, TIGAR, TTF1, UCHL1, UGT3A1, VEGFR2, WDR72, YAP_pS127, and ZMAT4;
AKR1C2, AKR1C4, C12orf39, C12orf56, C20orf70, CALB1, CPS1, CSAG2, F2, F7, GNG4, HEPACAM2, HOXD11, HOXD13, IGF2BP1, INSL4, KCNU1, KLK14, LOC100190940, MLLT11, PCSK1, POPDC3, SLC38A8, UCHL1, UGT3A1, WDR72, and ZMAT4;
ACETYLATUBULINLYS40, AMPKALPHA, ANNEXIN1, BIM, CASPASE7CLEAVEDD198, CAVEOLIN1, CYCLINB1, JNK2, MIG6, MSH6, MTOR_pS2448, NAPSINA, NCADHERIN, NRF2, P38MAPK, P90RSK, PEA15, PKCALPHA_pS657, SYNAPTOPHYSIN, TFRC, TIGAR, TTF1, VEGFR2, and YAP_pS127; and
BIM, CAVEOLIN1, FOXM1, NRF2, and PKCPANBETAII_pS660;
c) a panel for characterizing an S2 subtype lung adenocarcinoma in a biological sample of a subject, the panel comprising two or more polypeptide or polynucleotide markers, or fragments thereof, selected from the groups consisting of:
SLC24A2, COL8A2, C7orf10, CYP26A1, and MMP11;
ARAF_pS299, BAP1C4, BIM, C7orf10, CAPNS2, CASPASE7CLEAVEDD198, CILP2, CLAUDIN7, CMET_pY1235, COL8A2, CXorf64, CYCLINE1, CYP26A1, DBC1, DIO1, EGFR_pY1068, ENPP3, FIBRONECTIN, FNDC1, GPR88, IBSP, INPP4B, ISM1, ITGA11, LIPK, LRRTM1, MAPK_pT202Y204, MATN3, MFAP5, MMP11, MYO3B, MYOSINIIA_pS1943, P21, P27, P63, PAXILLIN, PCDH19, PCDH8, PCNA, PLAT, PODNL1, PRND, RANBP3L, SHISA3, SHP2_pY542, SLC24A2, SMAD4, SPP1, ST8SIA2, THBS2, and ZPLD1;
SLC24A2, C7orf10, MFAP5, GPR88, MATN3, FNDC1, RANBP3L, CILP2, PCDH19, SPP1, CAPNS2, ZPLD1, ENPP3, PRND, PLAT, PODNL1, LIPK, SHISA3, CXorf64, DIO1, PCDH8, DBC1, and MYO3B;
BIM, CMET_pY1235, CASPASE7CLEAVEDD198, CLAUDIN7, CYCLINE1, EGFR_pY1068, FIBRONECTIN, INPP4B, MAPK_pT202Y204, P27, PAXILLIN, PCNA, SMAD4, ARAF_pS299, BAP1C4, MYOSINIIA_pS1943, P21, SHP2_pY542, and P63; and
BIM, CLAUDIN7, EGFR_pY1068, P27, and ARAF_pS299;
d) a panel for characterizing a lung cancer in a biological sample of a subject, the panel comprising two or more polypeptide or polynucleotide markers, or fragments thereof, selected from those listed in Table 1; or
e) a panel of capture molecules, wherein each capture molecule binds a polypeptide or polynucleotide marker recited in a panel of any one of a-d.

139. The panel of claim 138, wherein the polypeptide or polynucleotide markers are each bound to a capture molecule.

140. The panel of claim 138, wherein each capture molecules comprise an antibody or antigen binding fragment thereof, or comprises a polynucleotide.

141. The panel of claim 138, wherein each capture molecule is bound to a substrate selected from the group consisting of chips, beads, microfluidic platforms, and membranes.

142. A method of treatment selected from the following:
a) a method of treating a subject selected as having a subtype 3 lung adenocarcinoma, the method comprising administering to the selected subject agents selected from:
a c-Met inhibitor and a CDK4/6 inhibitor;
a c-Met inhibitor and a PD-1 or a PD-L1 checkpoint inhibitor;
a CDK4/6 inhibitor and a PD-1 or a PD-L1 checkpoint inhibitor; or
a c-Met inhibitor, a CDK4/6 inhibitor, and a PD-1 or PD-L1 checkpoint inhibitor,
wherein the subject is selected by detecting in a biological sample obtained from the subject the level of one or more polypeptide or polynucleotide markers, or fragments thereof, selected from the group consisting of:
AIM2, CD274, DCBLD2, FBXO32, and MYBL1;
AFAP1L2, AIM2, ANNEXINVII, ARNTL2, BATF3, BRD4, C10orf55, C12orf70, C15orf48, CATSPER1, CD20, CD274, CD70, CD8A, CDA, CHK1_pS345, CSF2, DCBLD2, DJ1, ERALPHA, FBXO32, GATA3, GATA6, GBP1, GPR84, GZMB, IFNG, JAK2, KCNK12, LCK, MET, MIG6, MYBL1, NKG7, P63, P70S6K1, PDCD1, PDL1, PEA15, PI3KP110ALPHA, PKCDELTA_pS664, S100A2, SYNAPTOPHYSIN, TBX21, TGM4, TIGAR, TMEM156, and TTF1;
AFAP1L2, ARNTL2, BATF3, C15orf48, CATSPER1, CD274, CD70, CD8A, CDA, CSF2, DCBLD2, FBXO32, GPR84, GZMB, KCNK12, MET, MYBL1, NKG7, S100A2, TBX21, TGM4, and TMEM156;
ANNEXINVII, BRD4, CD20, CHK1_pS345, DJ1, ERALPHA, GATA3, GATA6, JAK2, LCK, MIG6, P63, PDCD1, PDL1, PEA15, PI3KP110ALPHA, PKCDELTA_pS664, SYNAPTOPHYSIN, TIGAR, and TTF1; and
GATA6, JAK2, MIG6, P70S6K1, and PDL1;
b) a method of treating a subject selected as having a subtype 4 lung adenocarcinoma, the method comprising administering to the selected subject a c-Met inhibitor and/or a CDK4/6 inhibitor, wherein the subject is selected by detecting in a biological sample obtained from the subject the level of one or more polypeptide or polynucleotide markers, or fragments thereof selected from the group consisting of:
AKR1C4, CALCA, HOXD13, MLLT11, and PAH;
ACETYLATUBULINLYS40, AKR1C2, AKR1C4, AMPKALPHA, ANNEXIN1, BIM, C12orf39, C12orf56, C20orf70, CALB1, CALCA, CASPASE7CLEAVEDD198, CAVEOLIN1, CPS1, CSAG2, CYCLINB1, DUSP4, F2, F7, FOXM1, GLDC, GNG4, HEPACAM2, HOXD11, HOXD13, IGF2BP1, INSL4, JNK2, KCNU1, KLK14, LOC100190940, LOC441177, MIG6, MLLT11, MSH6, MTOR_pS2448, NAPSINA, NCADHERIN, NRF2, P38MAPK, P90RSK, PAH, PCSK1, PEA15, PKCALPHA_pS657, PKCPANBETAII_pS660, POPDC3, SLC38A8, SYNAPTOPHYSIN, TFRC, TIGAR, TTF1, UCHL1, UGT3A1, VEGFR2, WDR72, YAP_pS127, and ZMAT4;
AKR1C2, AKR1C4, C12orf39, C12orf56, C20orf70, CALB1, CPS1, CSAG2, F2, F7, GNG4, HEPACAM2, HOXD11, HOXD13, IGF2BP1, INSL4, KCNU1, KLK14, LOC100190940, MLLT11, PCSK1, POPDC3, SLC38A8, UCHL1, UGT3A1, WDR72, and ZMAT4;
ACETYLATUBULINLYS40, AMPKALPHA, ANNEXIN1, BIM, CASPASE7CLEAVEDD198, CAVEOLIN1, CYCLINB1, JNK2, MIG6, MSH6, MTOR_pS2448, NAPSINA, NCADHERIN, NRF2, P38MAPK, P90RSK, PEA15, PKCALPHA_pS657, SYNAPTOPHYSIN, TFRC, TIGAR, TTF1, VEGFR2, and YAP_pS127; and
BIM, CAVEOLIN1, FOXM1, NRF2, and PKCPANBETAII_pS660; or
c) a method of treating a subject selected as having a subtype 2 lung adenocarcinoma, the method comprising administering to the selected subject an EGFR inhibitor and/or a TGF-beta inhibitor, wherein the subject is selected by detecting in a biological sample obtained from the subject the level of one or more polypeptide or polynucleotide markers, or fragments thereof, selected from the group consisting of:
SLC24A2, COL8A2, C7orf10, CYP26A1, and MMP11;
ARAF_pS299, BAP1C4, BIM, C7orf10, CAPNS2, CASPASE7CLEAVEDD198, CILP2, CLAUDIN7, CMET_pY1235, COL8A2, Cxorf64, CYCLINE1, CYP26A1, DBC1, DIO1, EGFR_pY1068, ENPP3, FIBRONECTIN, FNDC1, GPR88, IBSP, INPP4B, ISM1, ITGA11, LIPK, LRRTM1, MAPK_pT202Y204, MATN3, MFAP5, MMP11, MYO3B, MYOSINIIA_pS1943, P21, P27, P63, PAXILLIN, PCDH19, PCDH8, PCNA, PLAT, PODNL1, PRND, RANBP3L, SHISA3, SHP2_pY542, SLC24A2, SMAD4, SPP1, ST8SIA2, THBS2, and ZPLD1; and
SLC24A2, C7orf10, MFAP5, GPR88, MATN3, FNDC1, RANBP3L, CILP2, PCDH19, SPP1, CAPNS2, ZPLD1, ENPP3, PRND, PLAT, PODNL1, LIPK, SHISA3, Cxorf64, DIO1, PCDH8, DBC1, and MYO3B;
BIM, CMET_pY1235, CASPASE7CLEAVEDD198, CLAUDIN7, CYCLINE1, EGFR_pY1068, FIBRONECTIN, INPP4B, MAPK_pT202Y204, P27, PAXILLIN, PCNA, SMAD4, ARAF_pS299, BAP1C4, MYOSINIIA_pS1943, P21, SHP2_pY542, and P63; and
BIM, CLAUDIN7, EGFR_pY1068, P27, and ARAF_pS299.

143. The method of claim 142, wherein the polypeptide or polynucleotide markers are bound to a capture molecule.

144. The method of claim 143, wherein the capture molecules comprise an antibody or antigen binding fragment thereof, or comprises a polynucleotide.

145. The method of claim 142, wherein the capture molecule is bound to a substrate selected from the group consisting of chips, beads, microfluidic platforms, and membranes.

146. The method of claim 142, further comprising using the detected level of the one or more polypeptide or polynucleotide markers or fragments thereof to classify the selected subject, wherein the classification has an accuracy of at least about 80%.

147. The method of claim 142, wherein the subject selected as having a subtype 2 lung adenocarcinoma is additionally treated with an EGFR inhibitor and a TGF-beta inhibitor.

148. The method of claim 142, wherein in the method of treating a subject selected as having a subtype 2 lung adenocarcinoma, the EGFR inhibitor is selected from the group consisting of Erlotinib, Osimertinib, Neratinib, Gefitinib, Cetuximab, Panitumumab, Dacomitinib, Lapatinib, Necitumumab, Mobocertinib, Vandetanib, and pharmaceutically acceptable salts thereof.

149. The method of claim 142, wherein in the method of treating a subject selected as having a subtype 2 lung adenocarcinoma, the TGF-beta inhibitor is selected from the group consisting of Galunisertib, Vactosertib, Trabedersen, ISTH0036, Fresolimumab, Disitertide, Belagenpumatucel-L, Gemogenovatucel-T, and pharmaceutically acceptable salts thereof.

150. The method of claim 142, wherein in the method of treating a subject selected as having a subtype 3 or in the method of treating a subject selected as having a subtype 4 lung adenocarcinoma, the c-Met inhibitor is selected from the group consisting of AMG337, BMS 777607, cabozantinib, capmatinib, crizotinib, emibetuzumab, ficlatuzumab, foretinib, glesatinib, onartuzumab, rilotumumab, tepotinib, tivantinib, volitinib, and pharmaceutically acceptable salts thereof.

151. The method of claim 142, wherein in the method of treating a subject selected as having a subtype 3 or in the method of treating a subject selected as having a subtype 4 lung adenocarcinoma, the CDK4/6 inhibitor is selected from the group consisting of abemaciclib, AT7519, CINK4, flavopiridol, palbociclib, ribociclib, and pharmaceutically acceptable salts thereof.

152. The method of claim 142, wherein in the method of treating a subject selected as having a subtype 3, the PD-1/PD-L1 checkpoint inhibitor is selected from the group consisting of atezolizumab, avelumab, BMS-936559, MDX-1105, cemiplimab, durvalumab, nivolumab, pembrolizumab, and pharmaceutically acceptable salts thereof.

153. The method of claim 142, further comprising administering to the subject at least one additional chemotherapeutic agent.

154. A method for selecting a subject for inclusion in or exclusion from a clinical trial of an agent for the treatment of a lung adenocarcinoma, the method comprising:
when the lung adenocarcinoma is an S3 subtype, detecting in a biological sample obtained from the subject the level of one or more polypeptide or polynucleotide markers, or fragments thereof, selected from the group consisting of:
AIM2, CD274, DCBLD2, FBXO32, and MYBL1;
AFAP1L2, AIM2, ANNEXINVII, ARNTL2, BATF3, BRD4, C10orf55, C12orf70, C15orf48, CATSPER1, CD20, CD274, CD70, CD8A, CDA, CHK1_pS345, CSF2, DCBLD2, DJ1, ERALPHA, FBXO32, GATA3, GATA6, GBP1, GPR84, GZMB, IFNG, JAK2, KCNK12, LCK, MET, MIG6, MYBL1, NKG7, P63, P70S6K1, PDCD1, PDL1, PEA15, PI3KP110ALPHA, PKCDELTA_pS664, S100A2, SYNAPTOPHYSIN, TBX21, TGM4, TIGAR, TMEM156, and TTF1;
AFAP1L2, ARNTL2, BATF3, C15orf48, CATSPER1, CD274, CD70, CD8A, CDA, CSF2, DCBLD2, FBXO32, GPR84, GZMB, KCNK12, MET, MYBL1, NKG7, S100A2, TBX21, TGM4, and TMEM156;
ANNEXINVII, BRD4, CD20, CHK1_pS345, DJ1, ERALPHA, GATA3, GATA6, JAK2, LCK, MIG6, P63, PDCD1, PDL1, PEA15, PI3KP110ALPHA, PKCDELTA_pS664, SYNAPTOPHYSIN, TIGAR, and TTF1; and
GATA6, JAK2, MIG6, P70S6K1, and PDL1;
when the lung adenocarcinoma is an S4 subtype, detecting in a biological sample obtained from the subject the level of one or more polypeptide or polynucleotide markers, or fragments thereof, the selected from the groups consisting of:
AKR1C4, CALCA, HOXD13, MLLT11, and PAH;
ACETYLATUBULINLYS40, AKR1C2, AKR1C4, AMPKALPHA, ANNEXIN1, BIM, C12orf39, C12orf56, C20orf70, CALB1, CALCA, CASPASE7CLEAVEDD198, CAVEOLIN1, CPS1, CSAG2, CYCLINB1, DUSP4, F2, F7, FOXM1, GLDC, GNG4, HEPACAM2, HOXD11, HOXD13, IGF2BP1, INSL4, JNK2, KCNU1, KLK14, LOC100190940, LOC441177, MIG6, MLLT11, MSH6, MTOR_pS2448, NAPSINA, NCADHERIN, NRF2, P38MAPK, P90RSK, PAH, PCSK1, PEA15, PKCALPHA_pS657, PKCPANBETAII_pS660, POPDC3, SLC38A8, SYNAPTOPHYSIN, TFRC, TIGAR, TTF1, UCHL1, UGT3A1, VEGFR2, WDR72, YAP_pS127, and ZMAT4;
AKR1C2, AKR1C4, C12orf39, C12orf56, C20orf70, CALB1, CPS1, CSAG2, F2, F7, GNG4, HEPACAM2, HOXD11, HOXD13, IGF2BP1, INSL4, KCNU1, KLK14, LOC100190940, MLLT11, PCSK1, POPDC3, SLC38A8, UCHL1, UGT3A1, WDR72, and ZMAT4;
ACETYLATUBULINLYS40, AMPKALPHA, ANNEXIN1, BIM, CASPASE7CLEAVEDD198, CAVEOLIN1, CYCLINB1, JNK2, MIG6, MSH6, MTOR_pS2448, NAPSINA, NCADHERIN, NRF2, P38MAPK, P90RSK, PEA15, PKCALPHA_pS657, SYNAPTOPHYSIN, TFRC, TIGAR, TTF1, VEGFR2, and YAP_pS127;
BIM, CAVEOLIN1, FOXM1, NRF2, and PKCPANBETAII_pS660; and
when the lung adenocarcinoma is an S2 subtype lung adenocarcinoma, detecting in a biological sample obtained from the subject, the level of one or more polypeptide or polynucleotide markers, or fragments thereof, selected from the groups consisting of:
SLC24A2, COL8A2, C7orf10, CYP26A1, and MMP11;
ARAF_pS299, BAP1C4, BIM, C7orf10, CAPNS2, CASPASE7CLEAVEDD198, CILP2, CLAUDIN7, CMET_pY1235, COL8A2, CXorf64, CYCLINE1, CYP26A1, DBC1, DIO1, EGFR_pY1068, ENPP3, FIBRONECTIN, FNDC1, GPR88, IBSP, INPP4B, ISM1, ITGA11, LIPK, LRRTM1, MAPK_pT202Y204, MATN3, MFAP5, MMP11, MYO3B, MYOSINIIA_pS1943, P21, P27, P63, PAXILLIN, PCDH19, PCDH8, PCNA, PLAT, PODNL1, PRND, RANBP3L, SHISA3, SHP2_pY542, SLC24A2, SMAD4, SPP1, ST8SIA2, THBS2, and ZPLD1; and
SLC24A2, C7orf10, MFAP5, GPR88, MATN3, FNDC1, RANBP3L, CILP2, PCDH19, SPP1, CAPNS2, ZPLD1, ENPP3, PRND, PLAT, PODNL1, LIPK, SHISA3, CXorf64, DIO1, PCDH8, DBC1, and MYO3B;
BIM, CMET_pY1235, CASPASE7CLEAVEDD198, CLAUDIN7, CYCLINE1, EGFR_pY1068, FIBRONECTIN, INPP4B, MAPK_pT202Y204, P27, PAXILLIN, PCNA, SMAD4, ARAF_pS299, BAP1C4, MYOSINIIA_pS1943, P21, SHP2_pY542, and P63; and
BIM, CLAUDIN7, EGFR_pY1068, P27, and ARAF_pS299;
wherein the level of one or more polypeptide or polynucleotide markers or fragments thereof is detected relative to a corresponding reference level, and
wherein detecting an alteration in the level relative to the reference level indicates that the subject is a good candidate for inclusion in the clinical trial.

155. The method of claim 154, wherein in a clinical trial when the lung adenocarcinoma is an S4 subtype, the agent comprises:
a c-Met inhibitor selected from the group consisting of AMG337, BMS 777607, cabozantinib, capmatinib, crizotinib, emibetuzumab, ficlatuzumab, foretinib, glesatinib, onartuzumab, rilotumumab, tepotinib, tivantinib, volitinib, and pharmaceutically acceptable salts thereof;
a CDK4/6 inhibitor selected from the group consisting of abemaciclib, AT7519, CINK4, flavopiridol, palbociclib, ribociclib, and pharmaceutically acceptable salts thereof; and/or
a PD-1/PD-L1 checkpoint inhibitor selected from the group consisting of atezolizumab, avelumab, BMS-936559, MDX-1105, cemiplimab, durvalumab, nivolumab, pembrolizumab, and pharmaceutically acceptable salts thereof.

156. The method of claim 154, wherein the agent comprises:
an EGFR inhibitor selected from the group consisting of Erlotinib, Osimertinib, Neratinib, Gefitinib, Cetuximab, Panitumumab, Dacomitinib, Lapatinib, Necitumumab, Mobocertinib, Vandetanib, and pharmaceutically acceptable salts thereof;
a TGF-beta inhibitor selected from the group consisting of Galunisertib, Vactosertib, Trabedersen, ISTH0036, Fresolimumab, Disitertide, Lucanix™, Gemogenovatucel-T, and pharmaceutically acceptable salts thereof;
a c-Met inhibitor selected from the group consisting of AMG337, BMS 777607, cabozantinib, capmatinib, crizotinib, emibetuzumab, ficlatuzumab, foretinib, glesatinib, onartuzumab, rilotumumab, tepotinib, tivantinib, volitinib, and pharmaceutically acceptable salts thereof;
a CDK4/6 inhibitor selected from the group consisting of abemaciclib, AT7519, CINK4, flavopiridol, palbociclib, ribociclib, and pharmaceutically acceptable salts thereof; and/or
a PD-1/PD-L1 checkpoint inhibitor selected from the group consisting of atezolizumab, avelumab, BMS-936559, MDX-1105, cemiplimab, durvalumab, nivolumab, pembrolizumab, and pharmaceutically acceptable salts thereof.

157. The method of claim 154, wherein the level of at least 5 of the polypeptide or polynucleotide markers or fragments thereof is detected.
